(19) United States
(12) Patent Application Publication
(10) Pub. No.: US 2023/0413663 A1
(43) Pub. Date: Dec. 21, 2023

(54) LIGHT EMITTING ELEMENT AND AMINE COMPOUND FOR THE SAME

(71) Applicant: Samsung Display Co., Ltd., Yongin-si (KR)

(72) Inventors: MINJI KIM, Yongin-si (KR); HANKYU PAK, Yongin-si (KR); BYEONGWOOK YOO, Yongin-si (KR); JEONGMIN LEE, Yongin-si (KR); SOHEE JO, Yongin-si (KR)

(21) Appl. No.: 18/312,457

(22) Filed: May 4, 2023

(30) Foreign Application Priority Data
Jun. 20, 2022 (KR) 10-2022-0074697

(51) Int. Cl. H10K 85/60; C07C 211/61

(52) U.S. Cl. CPC H10K 85/633; C07C 211/61; H10K 85/626; C07C 2602/10; C07C 2603/18; C07C 2603/40; C07C 2603/74; H10K 50/156

(57) ABSTRACT

A light emitting element of one or more embodiments may include a first electrode, a second electrode provided on the first electrode, and at least one functional layer provided between the first electrode and the second electrode. The at least one functional layer may include an amine compound of one or more embodiments, represented by Formula 1. Accordingly, the light emitting element of one or more embodiments may show a low driving voltage, high luminance, high efficiency and long-life characteristics.

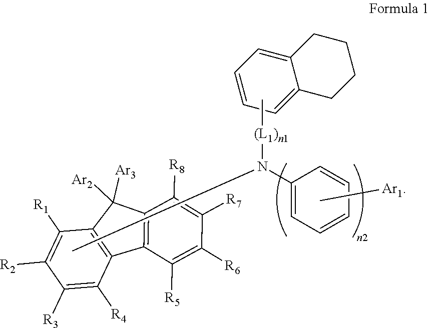

### CROSS-REFERENCE TO RELATED APPLICATIONS

This patent application claims priority to and the benefit of Korean Patent Application No. 10-2022-0074697, filed on Jun. 20, 2022, in the Korean Intellectual Property Office, the entire content of which is hereby incorporated by reference.

### BACKGROUND

### 1. Field

One or more aspects of embodiments of the present disclosure herein are directed toward a light emitting element and an amine compound used therein.

### 2. Description of Related Art

Recently, the development of an organic electroluminescence display device as an image display device is being actively conducted. The organic electroluminescence display device is a display device including a self-luminescent light emitting element in which holes and electrons injected from a first electrode and a second electrode recombine in an emission layer so that a light emitting material in the emission layer emits light to achieve display of images.

In the application of a light emitting element to a display device, the decrease of a driving voltage, the improvement of luminance and efficiency, and/or the increase of lifetime are required or desired, and development on materials for a light emitting element, capable of suitably achieving one or more these characteristics is being consistently required or desired.

### SUMMARY

One or more aspects of embodiments of the present disclosure are directed toward a light emitting element having a reduced driving voltage, improved luminance and efficiency, and increased lifetime.

One or more aspects of embodiments of the present disclosure are also directed toward an amine compound which is a material for a light emitting element having a reduced driving voltage, improved luminance and efficiency, and increased lifetime. Additional aspects will be set forth in part in the description which follows and, in part, will be apparent from the description, or may be learned by practice of the presented embodiments of the disclosure.

One or more embodiments provide a light emitting element including: a first electrode; a second electrode provided oppositely to the first electrode; and at least one functional layer provided between the first electrode and the second electrode, and including an amine compound represented by Formula 1:

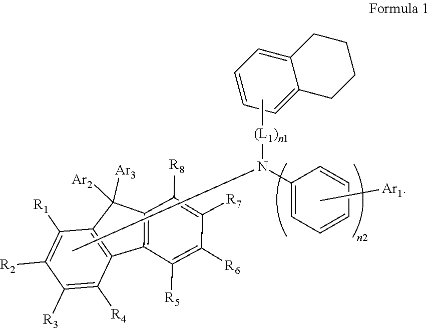

In Formula 1, n1 may be an integer of 0 to 2, L₁ may be a direct linkage, or an unsubstituted arylene group of 6 to 30 ring-forming carbon atoms, n2 may be 0 or 1, Ar₁ may be a substituted or unsubstituted methyl group, a substituted or unsubstituted cyclohexyl group, a substituted or unsubstituted norbornyl group, a substituted or unsubstituted adamantyl group, an unsubstituted phenyl group, an unsubstituted biphenyl group, a substituted or unsubstituted naphthyl group, or a substituted or unsubstituted terphenyl group, Ar₂ and Ar₃ may be each independently a substituted or unsubstituted silyl group, a substituted or unsubstituted alkoxy group of 1 to 30 carbon atoms, a substituted or unsubstituted alkyl group of 1 to 30 carbon atoms, a substituted or unsubstituted alkenyl group of 2 to 30 carbon atoms, a substituted or unsubstituted alkynyl group of 2 to 30 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and R₁ to R₈ may be each independently a hydrogen atom, a deuterium atom, a cyano group, a nitro group, a hydroxyl group, a halogen atom, a substituted or unsubstituted silyl group, a substituted or unsubstituted alkoxy group of 1 to 30 carbon atoms, a substituted or unsubstituted alkyl group of 1 to 30 carbon atoms, a substituted or unsubstituted alkenyl group of 2 to 30 carbon atoms, a substituted or unsubstituted alkynyl group or 2 to 30 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In one or more embodiments, in Formula 1, Ar₁ may be represented by any one of Formulae A1-1 to A1-8. Formula A1-1 represents an unsubstituted methyl group, and Formula A1-2 represents an unsubstituted cyclohexyl group. Formula A1-3 represents an unsubstituted norbornyl group, and Formula A1-4 represents an unsubstituted adamantyl group. Formula A1-5 represents an unsubstituted phenyl group, and Formula A1-6 represents an unsubstituted biphenyl group. Formula A1-7 represents a substituted or unsubstituted naphthyl group, and Formula A1-8 represents an unsubstituted terphenyl group.

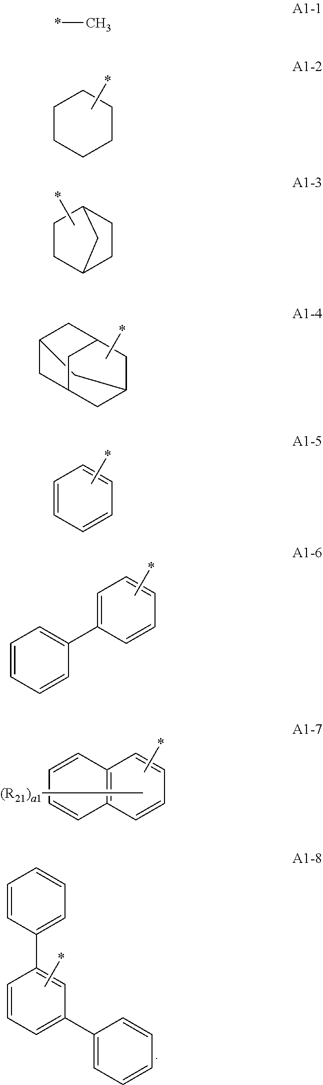

In Formula A1-7, a1 may be an integer of 0 to 7, and R₂₁ may be a hydrogen atom, a deuterium atom, or a substituted or unsubstituted aryl group of 6 to 30 ring-forming carbon atoms.

In one or more embodiments, Formula 1 may be represented by any one of Formula 1-1A to Formula 1-1C:

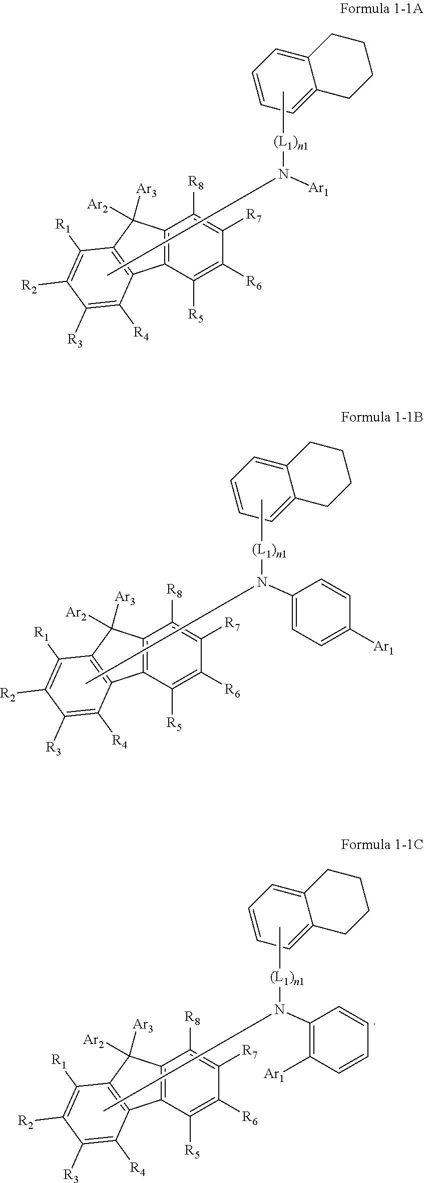

In Formula 1-1A to Formula 1-1C, Ar₁ to Ar₃, L₁, n1, and R₁ to R₈ are the same as defined in Formula 1.

In one or more embodiments, Formula 1 may be represented by any one of Formula 1-2A to Formula 1-2D:

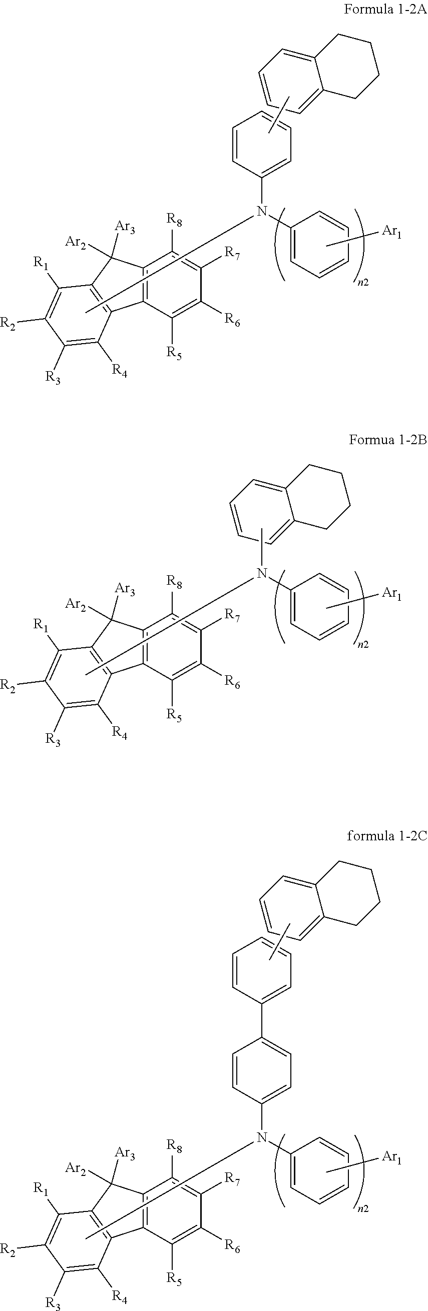

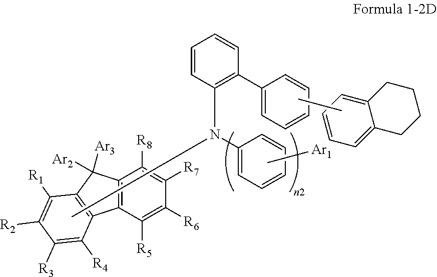

In Formula 1-2A to Formula 1-2D, Ar₁ to Ar₃, n2, and R₁ to R₈ are the same as defined in Formula 1.

In one or more embodiments, Formula 1-2A may be represented by any one of Formula 1-2AA and Formula 1-2AB:

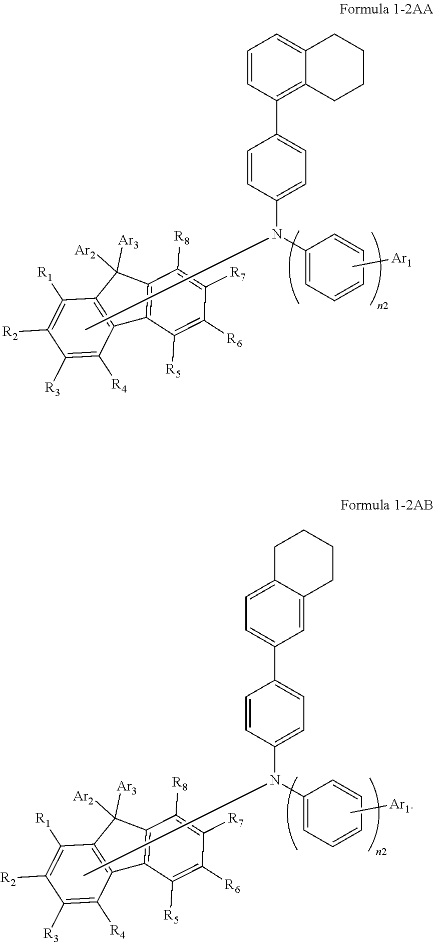

In Formula 1-2AA and Formula 1-2AB, Ar₁ to Ar₃, n2, and R₁ to R₈ are the same as defined in Formula 1-2A.

In one or more embodiments, Formula 1 may be represented by any one of Formula 1-3A to Formula 1-3C:

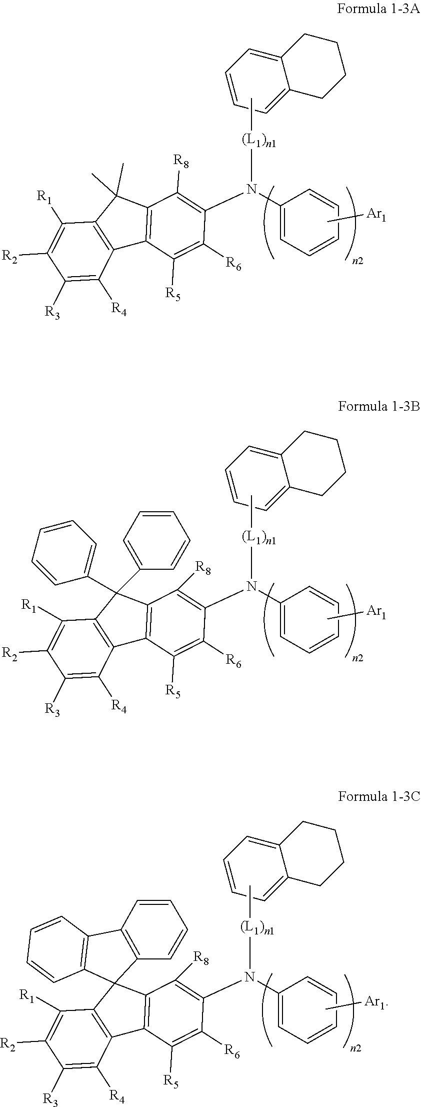

In Formula 1-3A to Formula 1-3C, Ar₁, L₁, n1, n2, and R₁ to R₈ are the same as defined in Formula 1.

In one or more embodiments, Formula 1-3A may be represented by any one of Formula 1-3AA to Formula 1-3AC:

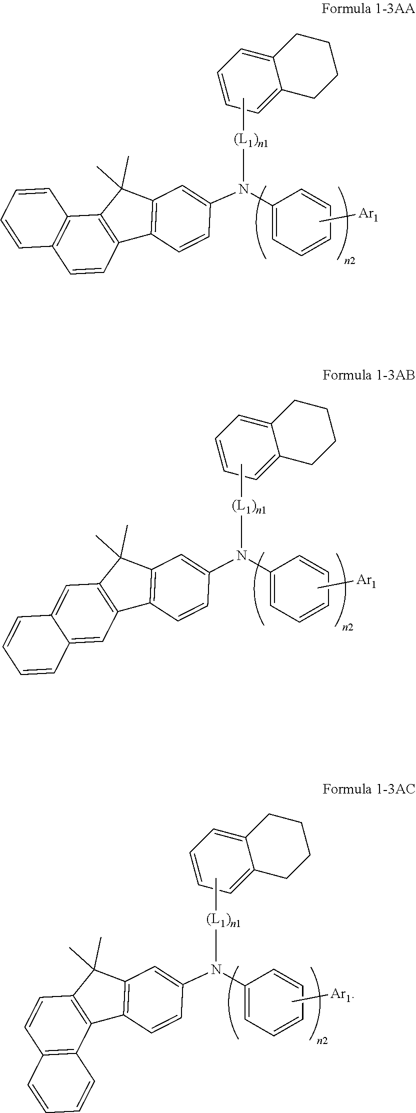

In Formula 1-3AA to Formula 1-3AC, Ar₁, L₁, n1, and n2 are the same as defined in Formula 1-3A.

In one or more embodiments, Formula 1-3A may be represented by Formula 1-3AD:

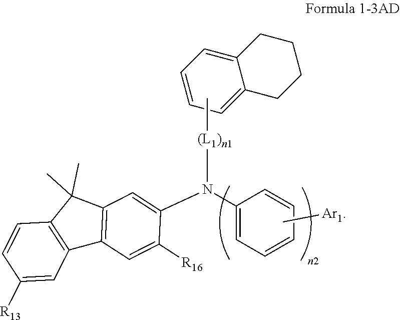

In Formula 1-3AD, R₁₃ and R₁₆ may be each independently a hydrogen atom, a substituted or unsubstituted methyl group, or a substituted or unsubstituted phenyl group, and Ar₁, L₁, n1 and n2 are the same as defined in Formula 1-3A.

In one or more embodiments, the at least one functional layer may include an emission layer, a hole transport region between the first electrode and the emission layer, and an electron transport region between the emission layer and the second electrode, and the hole transport region may include the amine compound.

In one or more embodiments, the hole transport region may include a hole injection layer on the first electrode, a hole transport layer on the hole injection layer, and an electron blocking layer on the hole transport layer, and at least one among the hole injection layer, the hole transport layer, or the electron blocking layer may include the amine compound.

According to one or more embodiments of the present disclosure, an amine compound represented by Formula 1 is provided.

### BRIEF DESCRIPTION OF THE DRAWINGS

The accompanying drawings are included to provide a further understanding of the present disclosure and are incorporated in and constitute a part of this specification. The drawings illustrate embodiments of the present disclosure and, together with the description, serve to explain principles of the present disclosure. In the drawings:

FIG. 1 is a plan view showing a display device according to one or more embodiments;

FIG. 2 is a cross-sectional view showing a part corresponding to line I-I′ in FIG. 1;

FIG. 3 is a cross-sectional view schematically showing a light emitting element of one or more embodiments;

FIG. 4 is a cross-sectional view schematically showing a light emitting element of one or more embodiments;

FIG. 5 is a cross-sectional view schematically showing a light emitting element of one or more embodiments;

FIG. 6 is a cross-sectional view schematically showing a light emitting element of one or more embodiments;

FIG. 7 is a cross-sectional view schematically showing a light emitting element of one or more embodiments;

FIG. 8 is a cross-sectional view of a display device according to one or more embodiments;

FIG. 9 is a cross-sectional view of a display device according to one or more embodiments;

FIG. 10 is a cross-sectional view showing a display device according to one or more embodiments; and

FIG. 11 is a cross-sectional view showing a display device according to one or more embodiments.

### DETAILED DESCRIPTION

The present disclosure may have various modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure.

In the description, when an element (or a region, a layer, a part, etc.) is referred to as being “on”, “connected with” or “combined with” another element, it can be directly connected with/bonded on the other element (e.g., without any intervening third elements therebetween), or intervening third elements may also be provided.

Like reference numerals refer to like elements throughout. In the drawings, the thicknesses, ratios, and dimensions of elements are exaggerated for effective explanation of technical contents. The term “and/or” may include any and all combinations that may define relevant elements.

It will be understood that, although the terms first, second, etc. may be used herein to describe various elements, these elements should not be limited by these terms. These terms are only used to distinguish one element from another element.

For example, a first element could be termed a second element without departing from the scope of the present invention. Similarly, a second element could be termed a first element. As used herein, the singular forms are intended to include the plural forms as well, unless the context clearly indicates otherwise.

In addition, the terms “below”, “beneath”, “on” and “above” are used for explaining the relation of elements shown in the drawings. The terms are relative concept and are explained based on the direction shown in the drawing. For example, if the device in the figures is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

It will be further understood that the terms “includes,” “including,” “comprises,” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

Unless otherwise defined, all terms (including technical and scientific terms) used herein have the same meaning as commonly understood by one of ordinary skill in the art to which this present disclosure belongs. In addition, it will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, expressions such as “at least one of”, “one of”, and “selected from”, when preceding a list of elements, modify the entire list of elements and do not modify the individual elements of the list. For example, “at least one selected from a, b and c”, “at least one of a, b or c”, “at least one of a to c”, and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

Further, the use of “may” when describing embodiments of the present disclosure refers to “one or more embodiments of the present disclosure.”

As used herein, the terms “substantially”, “about”, and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The display device and/or any other relevant devices or components according to embodiments of the present invention described herein may be implemented utilizing any suitable hardware, firmware (e.g. an application-specific integrated circuit), software, or a combination of software, firmware, and hardware. For example, the various components of the display device may be formed on one integrated circuit (IC) chip or on separate IC chips. Further, the various components of the display device may be implemented on a flexible printed circuit film, a tape carrier package (TCP), a printed circuit board (PCB), or formed on one substrate. Further, the various components of the display device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

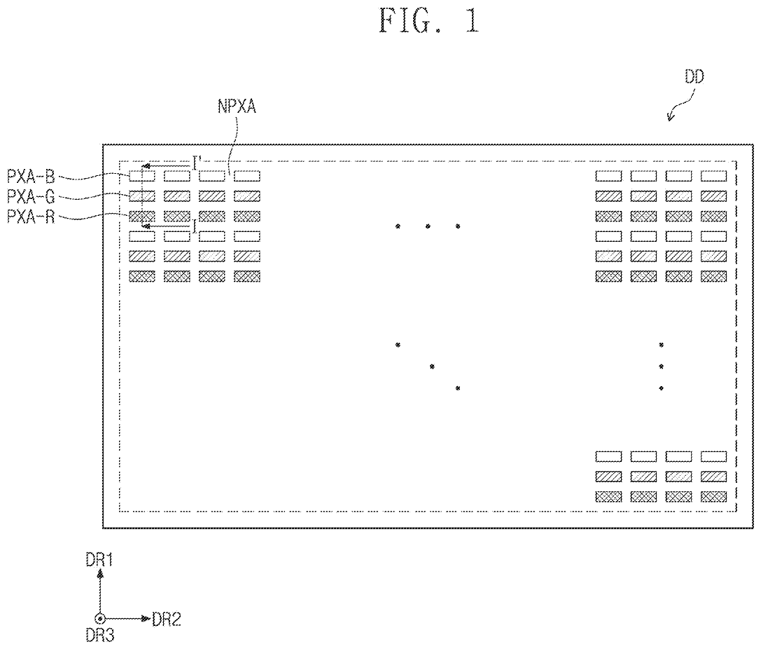

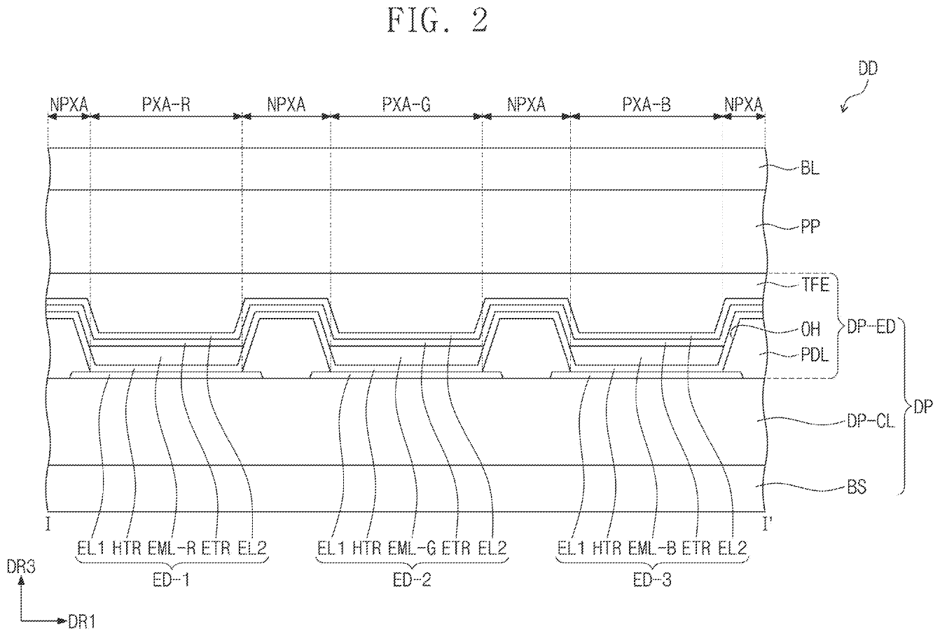

Hereinafter, embodiments of the present disclosure will be explained referring to the drawings. FIG. 1 is a plan view showing one or more embodiments of a display device DD. FIG. 2 is a cross-sectional view of a display device DD of one or more embodiments. FIG. 2 is a cross-sectional view showing a part corresponding to line I-I′ of FIG. 1.

The display device DD may include a display panel DP and an optical layer PP provided on the display panel DP. The display panel DP may include light emitting elements ED-1, ED-2 and ED-3. The display device DD may include multiple light emitting elements ED-1, ED-2 and ED-3. The optical layer PP may be provided on the display panel DP and may control reflected light by external light at the display panel DP. The optical layer PP may include, for example, a polarization layer and/or a color filter layer. In some embodiments, the optical layer PP may not be provided (e.g., may be omitted) in the display device DD of one or more embodiments.

On the optical layer PP, a base substrate BL may be provided. The base substrate BL may be a member providing a base surface where the optical layer PP is provided. The base substrate BL may be a glass substrate, a metal substrate, a plastic substrate, etc. However, one or more embodiments of the present disclosure is not limited thereto, and the base substrate BL may be an inorganic layer, an organic layer, or a composite material layer (e.g., including an inorganic material and an organic material). In some embodiments, the base substrate BL may not be provided (e.g., may be omitted) in one or more embodiments.

The display device DD according to one or more embodiments may further include a plugging layer. The plugging layer may be provided between a display element layer DP-ED and a base substrate BL. The plugging layer may be an organic layer. The plugging layer may include at least one of an acrylic resin, a silicon-based resin, and/or an epoxy-based resin.

The display panel DP may include a base layer BS, a circuit layer DP-CL provided on the base layer BS and a display element layer DP-ED. The display element layer DP-ED may include a pixel definition layer PDL, light emitting elements ED-1, ED-2 and ED-3 provided in the pixel definition layer PDL, and an encapsulating layer TFE provided on the light emitting elements ED-1, ED-2 and ED-3.

The base layer BS may be a member providing a base surface where the display element layer DP-ED is provided. The base layer BS may be a glass substrate, a metal substrate, a plastic substrate, etc. However, one or more embodiments of the present disclosure is not limited thereto, and the base layer BS may be an inorganic layer, an organic layer, or a composite material layer.

In one or more embodiments, the circuit layer DP-CL is provided on the base layer BS, and the circuit layer DP-CL may include one or more transistors. Each of the one or more transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

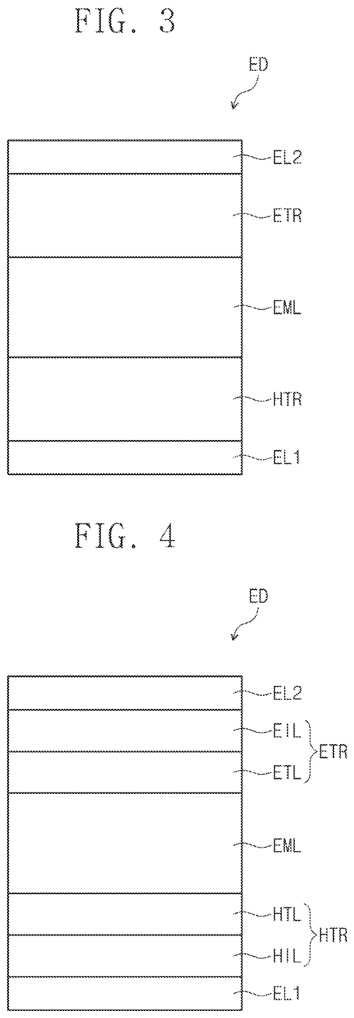

The light emitting elements ED-1, ED-2 and ED-3 may have the structures of the light emitting elements ED of embodiments according to FIG. 3 to FIG. 6, which will be explained in more detail herein below. The light emitting elements ED-1, ED-2 and ED-3 may include a first electrode EL1, a hole transport region HTR, emission layers EML-R, EML-G and EML-B, an electron transport region ETR and a second electrode EL2.

In FIG. 2, shown is an embodiment where the emission layers EML-R, EML-G and EML-B of light emitting elements ED-1, ED-2 and ED-3 are provided in opening portions OH defined in a pixel definition layer PDL, and a hole transport region HTR, an electron transport region ETR and a second electrode EL2 are provided as common layers in all light emitting elements ED-1, ED-2 and ED-3. However, one or more embodiments of the present disclosure is not limited thereto. In one or more embodiments, the hole transport region HTR and the electron transport region ETR may be patterned and provided in the opening portions OH defined in the pixel definition layer PDL. For example, in one or more embodiments, the hole transport region HTR, the emission layers EML-R, EML-G and EML-B, and the electron transport region ETR of the light emitting elements ED-1, ED-2 and ED-3 may be patterned by an ink jet printing method and provided in the opening portions OH defined in the pixel definition layer PDL.

An encapsulating layer TFE may cover the light emitting elements ED-1, ED-2 and ED-3. The encapsulating layer TFE may encapsulate the display element layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to one or more embodiments may include at least one inorganic layer (hereinafter, encapsulating inorganic layer). In some embodiments, the encapsulating layer TFE according to one or more embodiments may include at least one organic layer (hereinafter, encapsulating organic layer) and at least one encapsulating inorganic layer.

The encapsulating inorganic layer may protect the display element layer DP-ED from moisture/oxygen, and the encapsulating organic layer may protect the display element layer DP-ED from foreign materials such as dust particles. The encapsulating inorganic layer may include silicon nitride, silicon oxy nitride, silicon oxide, titanium oxide, and/or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be provided on the second electrode EL2 and may be provided while filling the opening portion OH.

Referring to FIG. 1 and FIG. 2, the display device DD may include a non-luminous area NPXA and luminous areas PXA-R, PXA-G and PXA-B. The luminous areas PXA-R, PXA-G and PXA-B may be areas emitting light produced from the light emitting elements ED-1, ED-2 and ED-3, respectively. The luminous areas PXA-R, PXA-G and PXA-B may be separated from each other on a plane (e.g., in a plan view).

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In the disclosure, the luminous areas PXA-R, PXA-G and PXA-B may respectively correspond to respective pixel(s). The pixel definition layer PDL may divide the light emitting elements ED-1, ED-2 and ED-3. The emission layers EML-R, EML-G and EML-B of the light emitting elements ED-1, ED-2 and ED-3 may be provided and divided in the opening portions OH defined in the pixel definition layer PDL.

The luminous areas PXA-R, PXA-G and PXA-B may be divided into multiple groups according to the color of light produced from the light emitting elements ED-1, ED-2 and ED-3. In the display device DD of one or more embodiments, shown in FIG. 1 and FIG. 2, three luminous areas PXA-R, PXA-G and PXA-B emitting red light, green light and blue light, respectively, are illustrated as one or more embodiments. For example, the display device DD of one or more embodiments may include a red luminous area PXA-R, a green luminous area PXA-G and a blue luminous area PXA-B, which are separated from each other.

In the display device DD according to one or more embodiments, multiple light emitting elements ED-1, ED-2 and ED-3 may emit light having different wavelength regions. For example, in one or more embodiments, the display device DD may include a first light emitting element ED-1 emitting (e.g., configured to emit) red light, a second light emitting element ED-2 emitting (e.g., configured to emit) green light, and a third light emitting element ED-3 emitting (e.g., configured to emit) blue light. For example, the red luminous area PXA-R, the green luminous area PXA-G, and the blue luminous area PXA-B of the display device DD may respectively correspond to the first light emitting element ED-1, the second light emitting element ED-2, and the third light emitting element ED-3.

However, one or more embodiments of the present disclosure is not limited thereto, and the first to third light emitting elements ED-1, ED-2 and ED-3 may emit light in the same wavelength region, or at least one thereof may emit light in a different wavelength region. For example, all the first to third light emitting elements ED-1, ED-2 and ED-3 may emit blue light.

The luminous areas PXA-R, PXA-G and PXA-B in the display device DD according to one or more embodiments may be arranged in a stripe shape or pattern.

Referring to FIG. 1, multiple red luminous areas PXA-R may be arranged with each other along a second directional axis DR2, multiple green luminous areas PXA-G may be arranged with each other along a second directional axis DR2, and multiple blue luminous areas PXA-B may be arranged with each other along a second directional axis DR2. In addition, the red luminous area PXA-R, the green luminous area PXA-G and the blue luminous area PXA-B may be arranged by turns (alternatingly with each other) along a first directional axis DR1.

In FIG. 1 and FIG. 2, the areas of the luminous areas PXA-R, PXA-G and PXA-B are shown to be similar, but one or more embodiments of the present disclosure is not limited thereto. The areas of the luminous areas PXA-R, PXA-G and PXA-B may be different from each other according to the wavelength region of light emitted. As used herein, the areas of the luminous areas PXA-R, PXA-G and PXA-B may mean areas on a plane (e.g., in a plan view) defined by the first directional axis DR1 and the second directional axis DR2.

In one or more embodiments, the arrangement type (or kind) of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in various suitable combinations according to the properties of display quality required or desired for the display device DD. For example, the arrangement type (or kind) of the luminous areas PXA-R, PXA-G and PXA-B may be a pentile (PENTILE©) arrangement type (or kind), or a diamond (Diamond Pixel™) arrangement type (or kind). PENTILE® is a registered trademark owned by Samsung Display Co., Ltd.

In some embodiments, the areas of the luminous areas PXA-R, PXA-G and PXA-B may be different from each other. For example, in one or more embodiments, the area of the green luminous area PXA-G may be smaller than the area of the blue luminous area PXA-B, but one or more embodiments of the present disclosure is not limited thereto.

Hereinafter, FIG. 3 to FIG. 7 are cross-sectional views schematically showing light emitting elements according to embodiments. The light emitting element ED according to one or more embodiments as shown in FIG. 3 may include a first electrode EL1, a hole transport region HTR, an emission layer EML, an electron transport region ETR, and a second electrode EL2, stacked in this order.

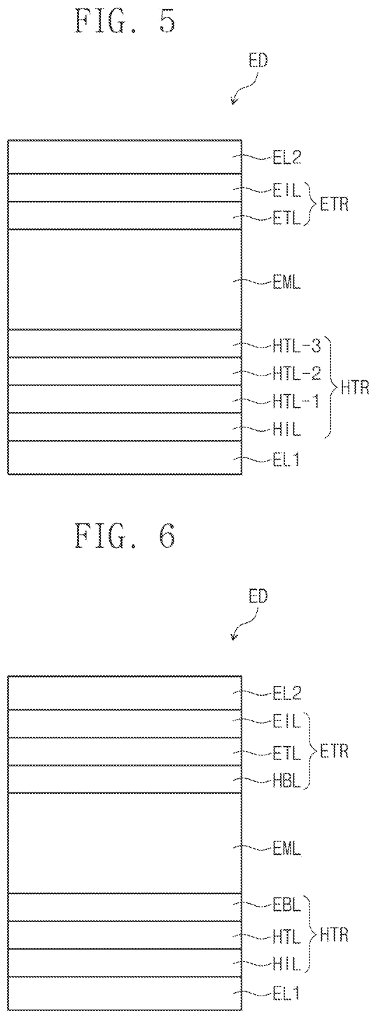

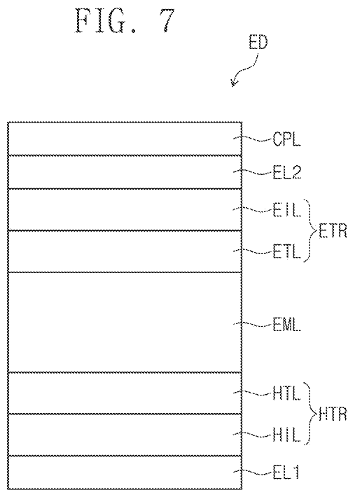

When compared with FIG. 3, FIG. 4 shows the cross-sectional view of a light emitting element ED of one or more embodiments, wherein a hole transport region HTR includes a hole injection layer HIL and a hole transport layer HTL, and an electron transport region ETR includes an electron injection layer EIL and an electron transport layer ETL. When compared with FIG. 4, FIG. 5 shows the cross-sectional view of a light emitting element ED of one or more embodiments, wherein a hole transport region HTR includes a hole injection layer HIL and first to third hole transport layers HTL-1, HTL-2 and HTL-3. When compared with FIG. 3, FIG. 6 shows the cross-sectional view of a light emitting element ED of one or more embodiments, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared with FIG. 4, FIG. 7 shows the cross-sectional view of a light emitting element ED of one or more embodiments, further including a capping layer CPL provided on the second electrode EL2.

The light emitting element ED of one or more embodiments may include an amine compound of one or more embodiments in at least one functional layer provided between the first electrode EL1 and the second electrode EL2. At least one functional layer may include a hole transport region HTR, an emission layer EML, and an electron transport region ETR. For example, the hole transport region HTR may include the amine compound of one or more embodiments.

The light emitting element ED including the amine compound of one or more embodiments may show a reduced driving voltage, high efficiency, high luminance and long-life characteristics. The amine compound of one or more embodiments may include one fluorene moiety and one tetrahydronaphthyl group bonded to the nitrogen atom of an amine group. The amine compound of one or more embodiments may include only one fluorene moiety. In the amine compound of one or more embodiments, the fluorene moiety may be directly bonded to the nitrogen atom of an amine group. The amine compound of one or more embodiments may include only one tetrahydronaphthyl group. In the amine compound of one or more embodiments, the tetrahydronaphthyl group may be directly bonded to the nitrogen atom of an amine group, or indirectly bonded via an unsubstituted arylene group. In the tetrahydronaphthyl group included in the amine compound of one or more embodiments, a benzene ring may be a position bonded to the nitrogen atom of the amine group.

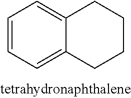

In the description, the term “substituted or unsubstituted” corresponds to a group that is unsubstituted or is substituted with at least one substituent selected from the group consisting of a deuterium atom, a halogen atom, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, a heterocyclic group, and combinations thereof. In addition, each of the exemplified substituents may itself be substituted or unsubstituted. For example, a biphenyl group may be interpreted as an aryl group or a phenyl group substituted with a phenyl group.

In the description, the term “forming a ring via the combination with an adjacent group” may mean forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

In the description, the term “adjacent group” may mean a pair of substituent groups where the first substituent is connected to an atom which is directly connected to another atom substituted with the second substituent; a pair of substituent groups connected to the same atom; or a pair of substituent groups where the first substituent is sterically positioned at the nearest position to the second substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the description, a halogen atom may be a fluorine atom, a chlorine atom, a bromine atom, and/or an iodine atom.

In the description, an alkyl group may be a linear, branched, or cyclic alkyl group. The carbon number of the alkyl group may be 1 to 60, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, an alkenyl group means a hydrocarbon group including one or more carbon double bonds in the middle and/or at the terminal of an alkyl group having a carbon number of 2 or more. The alkenyl group may be a linear chain or a branched chain. The carbon number is not specifically limited, but may be 2 to 60, 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the description, an alkynyl group means a hydrocarbon group including one or more carbon triple bonds in the middle and/or at the terminal of an alkyl group having a carbon number of 2 or more. The alkynyl group may be a linear chain or a branched chain. The carbon number is not specifically limited, but may be 2 to 60, 2 to 30, 2 to 20, or 2 to 10. Particular examples of the alkynyl group may include an ethynyl group, a propynyl group, etc., without limitation.

In the description, a hydrocarbon ring group means a functional group or substituent derived from an aliphatic hydrocarbon ring. The hydrocarbon ring group may be a saturated hydrocarbon ring group of 5 to 60, or 5 to 30, ring-forming carbon atoms.

In the description, an aryl group means a functional group or substituent derived from an aromatic hydrocarbon ring. The aryl group may be a monocyclic aryl group or a polycyclic aryl group. The carbon number for forming rings (e.g., ring-forming carbon atoms) in the aryl group may be 6 to 50, 6 to 30, 6 to 20, or 6 to 15.

Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the description, a fluorenyl group may be substituted, and two substituents may be combined with each other to form a spiro structure. Examples of a substituted fluorenyl group are as follows, but one or more embodiments of the present disclosure is not limited thereto:

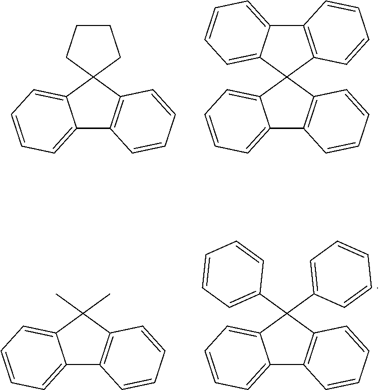

In the description, a heterocyclic group means a functional group or substituent derived from a ring including one or more of B, O, N, P, Si, and/or S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may each independently be a monocycle or a polycycle.

In the description, a heterocyclic group may include one or more of B, O, N, P, Si and/or S as heteroatoms. If the heterocyclic group includes two or more heteroatoms, two or more heteroatoms may be the same or different. The heterocyclic group may be a monocyclic heterocyclic group or polycyclic heterocyclic group and has concept including a heteroaryl group. The carbon number for forming rings (e.g., ring-forming carbon atoms) of the heterocyclic group may be 2 to 60, 2 to 30, 2 to 20, or 2 to 10.

In the description, an aliphatic heterocyclic group may include one or more of B, O, N, P, Si, and/or S as heteroatoms. The number of ring-forming carbon atoms of the aliphatic heterocyclic group may be 2 to 60, 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the description, a heteroaryl group may include one or more of B, O, N, P, Si, and/or S as heteroatoms. If the heteroaryl group includes two or more heteroatoms, two or more heteroatoms may be the same or different. The heteroaryl group may be a monocyclic heterocyclic group or polycyclic heterocyclic group. The carbon number for forming rings (e.g., ring-forming carbon atoms) of the heteroaryl group may be 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the same explanation on the above-described aryl group may be applied to an arylene group except that the arylene group is a divalent group.

The same explanation on the above-described heteroaryl group may be applied to a heteroarylene group except that the heteroarylene group is a divalent group.

In the description, a boron group may mean the above-defined alkyl group or aryl group bonded to a boron atom. The boron group may include an alkyl boron group and an aryl boron group. Examples of the boron group may include a dimethylboron group, a diethylboron group, a t-butylmethylboron group, a diphenylboron group, a phenylboron group, and the like, without limitation.

In the description, the carbon number of a carbonyl group is not specifically limited, but the carbon number may be 1 to 40, 1 to 30, or 1 to 20. For example, the carbonyl group may have the structure(s) below, but is not limited thereto:

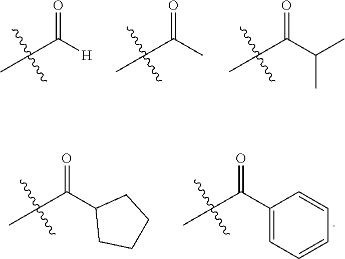

In the description, the carbon number of a sulfinyl group and sulfonyl group is not specifically limited, but may be 1 to 30. The sulfinyl group may include an alkyl sulfinyl group and an aryl sulfinyl group. The sulfonyl group may include an alkyl sulfonyl group and an aryl sulfonyl group.

In the description, a thio group may include an alkyl thio group and an aryl thio group. The thio group may mean the above-defined alkyl group or aryl group combined with a sulfur atom. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, an oxy group may mean the above-defined alkyl group or aryl group which is combined with an oxygen atom. The oxy group may include an alkoxy group and an aryl oxy group. The alkoxy group may be a linear, branched, or cyclic chain. The carbon number of the alkoxy group is not specifically limited but may be, for example, 1 to 30, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, one or more embodiments of the present disclosure is not limited thereto.

In the description, a silyl group may mean the above-defined alkyl group or aryl group bonded to a silicon atom. The silyl group includes an alkyl silyl group and an aryl silyl group. Examples of the silyl group may include a trimethylsilyl group, a triethylsilyl group, a t-butyldimethylsilyl group, ethyldimethylsilyl group, a propyldimethylsilyl group, a triphenylsilyl group, a diphenylsilyl group, a phenylsilyl group, etc., without limitation.

In the description, the carbon number of an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, etc., without limitation.

In the description, alkyl groups in an alkyl thio group, an alkyl sulfinyl group, an alkyl sulfonyl group, an alkoxy group, an alkyl boron group, an alkyl silyl group and an alkyl amine group may be the same as the examples of the above-described alkyl group.

In the description, aryl groups in an aryl oxy group, an aryl thio group, an aryl sulfinyl group, an aryl sulfonyl group, an aryl boron group, aryl silyl group, and an aryl amine group may be the same as the examples of the above-described aryl group.

In the description, a direct linkage may mean a single bond. In one or more embodiments, in the description,

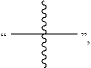

and “—*” mean positions to be connected (e.g., a bonding site).

The light emitting element ED of one or more embodiments may include the amine compound of one or more embodiments. The amine compound of one or more embodiments may be represented by Formula 1 below:

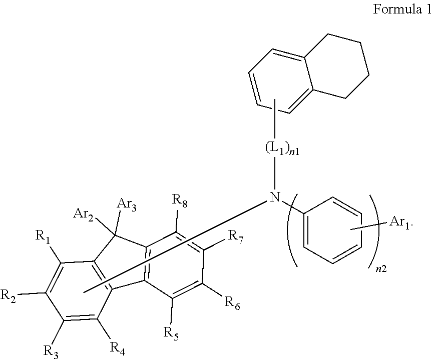

In Formula 1, n1 may be an integer of 0 to 2. L₁ may be a direct linkage, or an unsubstituted arylene group of 6 to 30 ring-forming carbon atoms. If n1 is 2, two L₁ may be the same or different. If n1 is 0, a tetrahydronaphthyl group may be directly bonded to the nitrogen atom of an amine group. If n1 is 2, L₁ may be an unsubstituted arylene group of 6 to 30 ring-forming carbon atoms. For example, if n1 is 1, L₁ may be an unsubstituted phenylene group or an unsubstituted divalent biphenyl group. If n1 is 2, L₁ may be an unsubstituted phenylene group. In the light emitting element ED including the amine compound of one or more embodiments, including the arylene group represented by L₁, the energy level difference between a hole transport region HTR and an emission layer EML may be suitably controlled to show high or suitable efficiency and long-life characteristics.

In Formula 1, n2 may be 0 or 1. If n2 is 0, Ar₁ may be directly bonded to the nitrogen atom of the amine group. If n2 is 1, Ar₁ may be indirectly bonded to the nitrogen atom of the amine group via a phenyl group.

Ar₁ may be a substituted or unsubstituted methyl group, a substituted or unsubstituted cyclohexyl group, a substituted or unsubstituted norbornyl group, a substituted or unsubstituted adamantyl group, an unsubstituted phenyl group, an unsubstituted biphenyl group, a substituted or unsubstituted naphthyl group, or a substituted or unsubstituted terphenyl group. For example, Ar₁ may be an unsubstituted methyl group, an unsubstituted cyclohexyl group, an unsubstituted norbornyl group, an unsubstituted adamantyl group, an unsubstituted phenyl group, an unsubstituted biphenyl group, a substituted or unsubstituted naphthyl group, or an unsubstituted terphenyl group.

Ar₂ and Ar₃ may be each independently a substituted or unsubstituted silyl group, a substituted or unsubstituted alkoxy group of 1 to 30 carbon atoms, a substituted or unsubstituted alkyl group of 1 to 30 carbon atoms, a substituted or unsubstituted alkenyl group of 2 to 30 carbon atoms, a substituted or unsubstituted alkynyl group of 2 to 30 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring. For example, Ar₂ and Ar₃ may be each independently a substituted or unsubstituted alkyl group of 1 to 10 carbon atoms, or a substituted or unsubstituted aryl group of 6 to 10 ring-forming carbon atoms. In some embodiments, Ar₂ and Ar₃ may be each independently a substituted or unsubstituted phenyl group, and Ar₂ and Ar₃ may be combined with each other to form a spirofluorenyl group.

R₁ to R₈ may be each independently a hydrogen atom, a deuterium atom, a cyano group, a nitro group, a hydroxyl group, a halogen atom, a substituted or unsubstituted silyl group, a substituted or unsubstituted alkoxy group of 1 to 30 carbon atoms, a substituted or unsubstituted alkyl group of 1 to 30 carbon atoms, a substituted or unsubstituted alkenyl group of 2 to 30 carbon atoms, a substituted or unsubstituted alkynyl group or 2 to 30 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring. For example, R₁ to R₈ may be hydrogen atoms, substituted or unsubstituted alkyl groups of 1 to 10 carbon atoms, substituted or unsubstituted alkenyl groups of 2 to 10 carbon atoms, or substituted or unsubstituted aryl groups of 6 to 10 ring-forming carbon atoms.

In one or more embodiments, R₁ and R₂ may be vinyl groups, and adjacent R₁ and R₂ may be combined to form a ring. R₂ and R₃ may be vinyl groups, and adjacent R₂ and R₃ may be combined to form a ring. R₃ and R₄ may be vinyl groups, and adjacent R₃ and R₄ may be combined to form a ring. In case of forming a ring by the combination of R₁ and R₂, R₂ and R₃, and/or R₃ and R₄, a fused ring of four rings, including a fluorene moiety, may be formed.

In one or more embodiments, Ar₁ may be represented by any one of A1-1 to A1-8. A1-1 represents an unsubstituted methyl group, and A1-2 represents an unsubstituted cyclohexyl group. A1-3 represents an unsubstituted norbornyl group, and A1-4 represents an unsubstituted adamantyl group. A1-5 represents an unsubstituted phenyl group, and A1-6 represents a biphenyl group. A1-7 represents a substituted or unsubstituted naphthyl group, and A1-8 represents an unsubstituted terphenyl group:

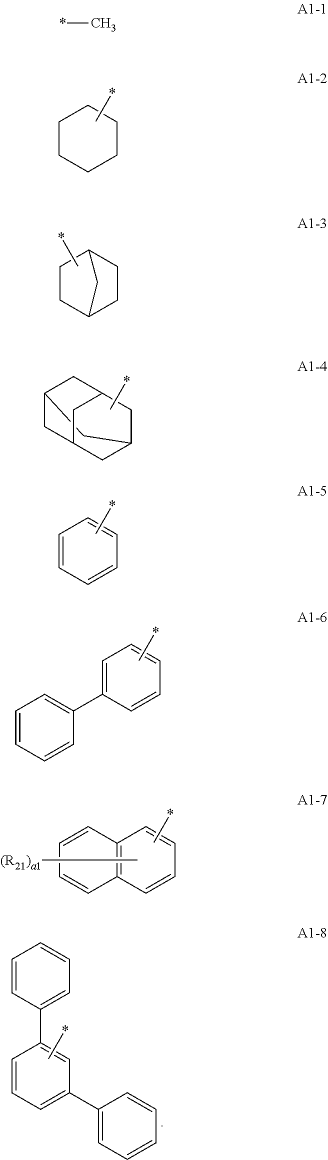

In A1-7, a1 may be an integer of 0 to 7. If a1 is an integer of 2 or more, multiple R₂₁ may be all the same, or at least one may be different. In A1-7, R₂₁ may be a hydrogen atom, a deuterium atom, or a substituted or unsubstituted aryl group of 6 to 30 ring-forming carbon atoms. For example, R₂₁ may be a hydrogen atom, an unsubstituted phenyl group, or an unsubstituted biphenyl group.

For example, Formula 1 may be represented by Formula 1-X. In Formula 1-X, Ar₁₁ corresponds to a phenyl group including n2 and Ar₁ in Formula 1 (e.g., a moiety represented by

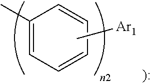

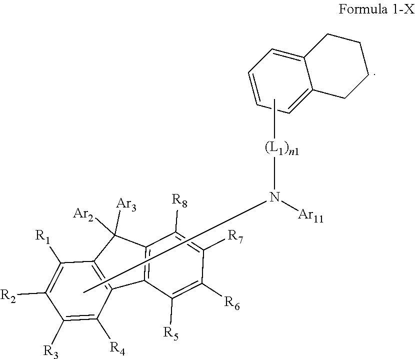

In Formula 1-X, the same descriptions as provided in Formula 1 may be applied for Ar₂, Ar₃, L₁, n1, and R₁ to R₈. In Formula 1-X, Ar₁₁ may be represented by any one of A11-1 to A11-25. In A11-1 to A11-25,

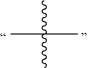

is a position bonded to the nitrogen atom of Formula 1.

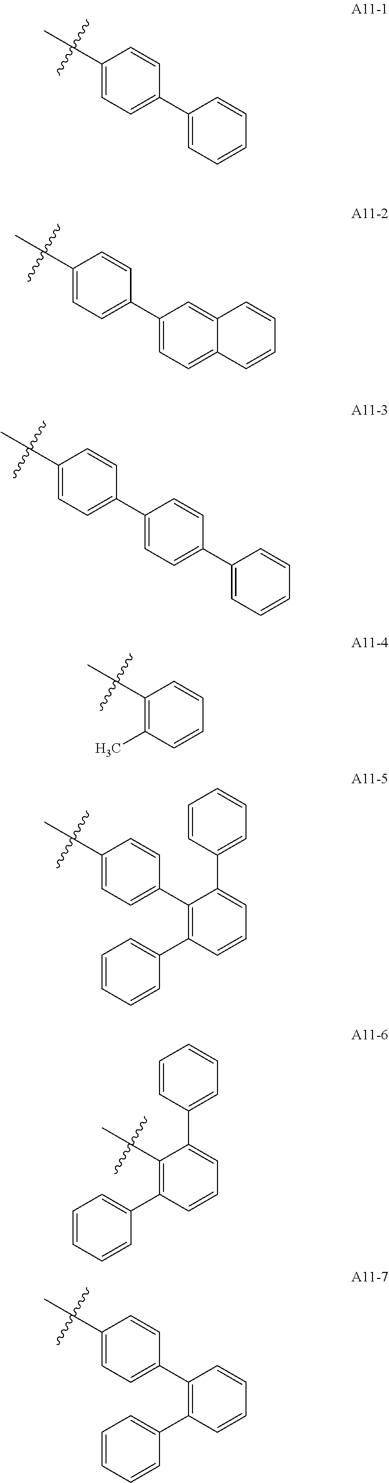

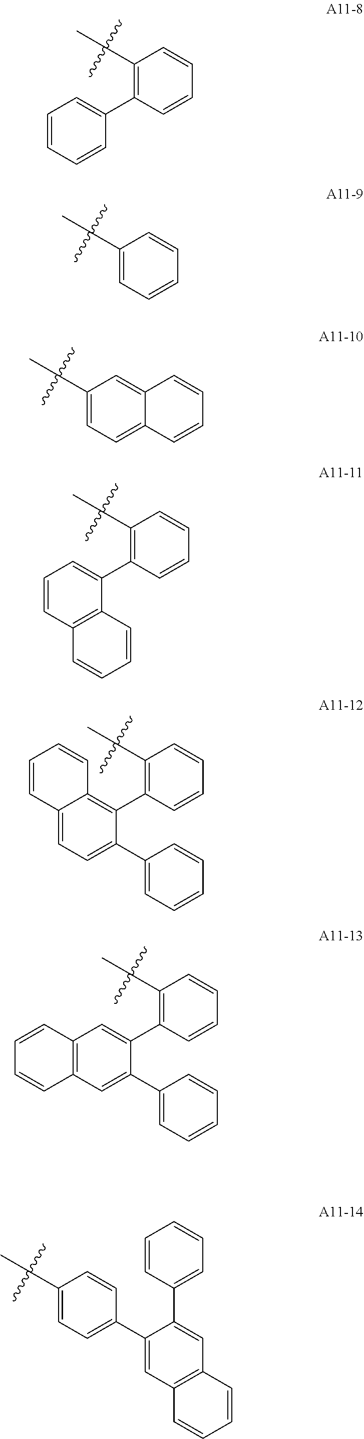

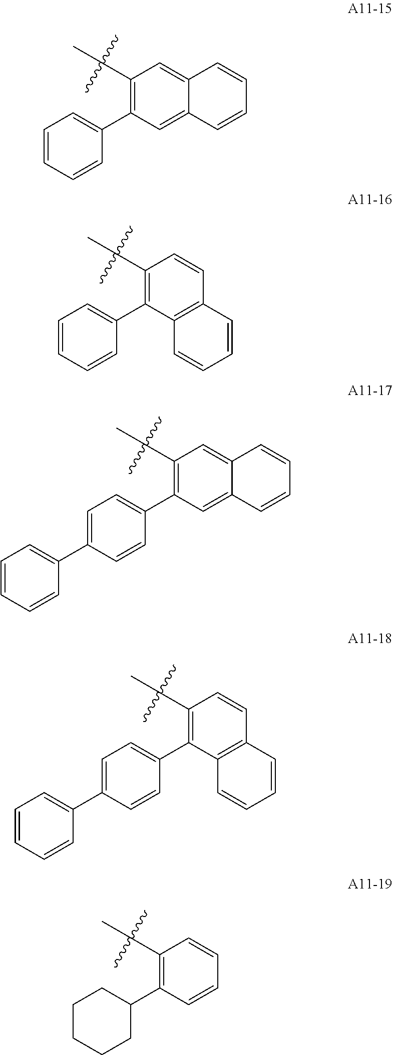 [SMILES: *c1cc2ccccc2cc1-c1ccccc1 ; *c1ccc2ccccc2c1-c1ccccc1 ; *c1cc2ccccc2cc1-c1ccc(-c2ccccc2)cc1 ; *c1ccc2ccccc2c1-c1ccc(-c2ccccc2)cc1 ; *c1ccccc1C1CCCCC1]

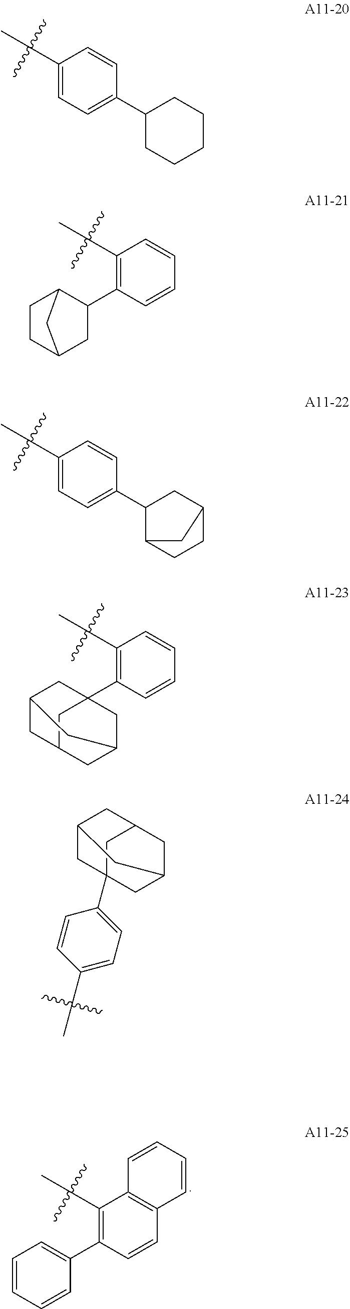 [SMILES: *c1ccc(C2CCCCC2)cc1 ; *c1ccccc1C1CC2CCC1C2 ; *c1ccc(C2CC3CCC2C3)cc1 ; *c1ccccc1C12CC3CC(CC(C3)C1)C2 ; *c1ccc(C23CC4CC(CC(C4)C2)C3)cc1 ; *c1c(-c2ccccc2)ccc2ccccc12]

In one or more embodiments, Formula 1 may be represented by any one of Formula 1-1A to Formula 1-1C. Formula 1-1A to Formula 1-1C represent Formula 1 where n2 is 0 or 1.

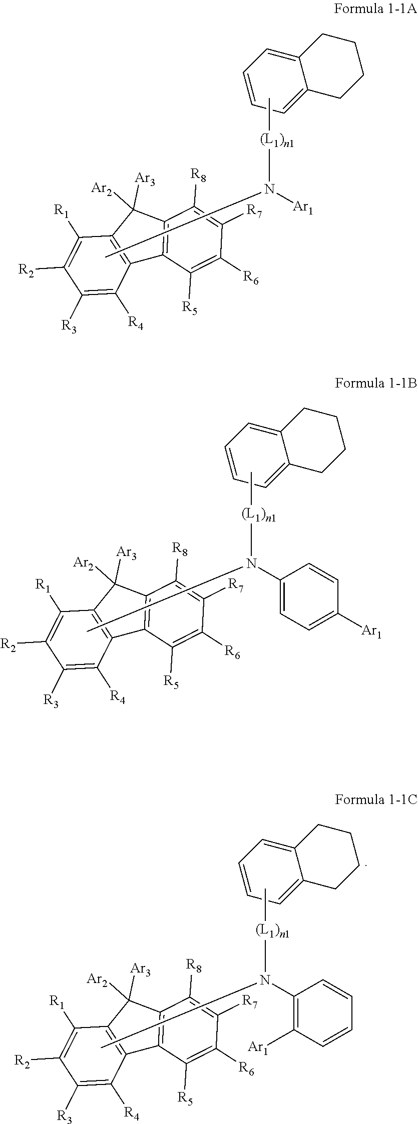

Formula 1-1A represents Formula 1 where n2 is 0. Formula 1-1B represents Formula 1 where n2 is 1, and Ar₁ has the para position relation with respect to the nitrogen atom. Formula 1-1C represents Formula 1 where n2 is 1, and Ar₁ has the ortho position relation with respect to the nitrogen atom. In Formula 1-1A to Formula 1-1C, the same descriptions as provided in Formula 1 may be applied for Ar₁ to Ar₃, L₁, n1, and R₁ to R₈.

For example, in Formula 1-1A, Ar₁ may be represented by any one of A11-1, A11-3, A11-6 to A11-10, A11-15 to A11-18, or A11-25. In Formula 1-1B, the phenyl group to which Ar₁ is bonded and Ar₁ may be represented by any one of A11-1 to A11-3, A11-5, A11-7, A11-14, A11-20, A11-22, or A11-24. In Formula 1-1C, the phenyl group to which Ar₁ is bonded and Ar₁ may be represented by any one of A11-4, A11-7, A11-8, A11-11 to A11-13, A11-19, A11-21, or A11-23.

In one or more embodiments, Formula 1 may be represented by any one of Formula 1-2A to Formula 1-2D. Formula 1-2A to Formula 1-2D represent cases where n1 is 0, and n1 is 1 or 2, with L₁ embodied.

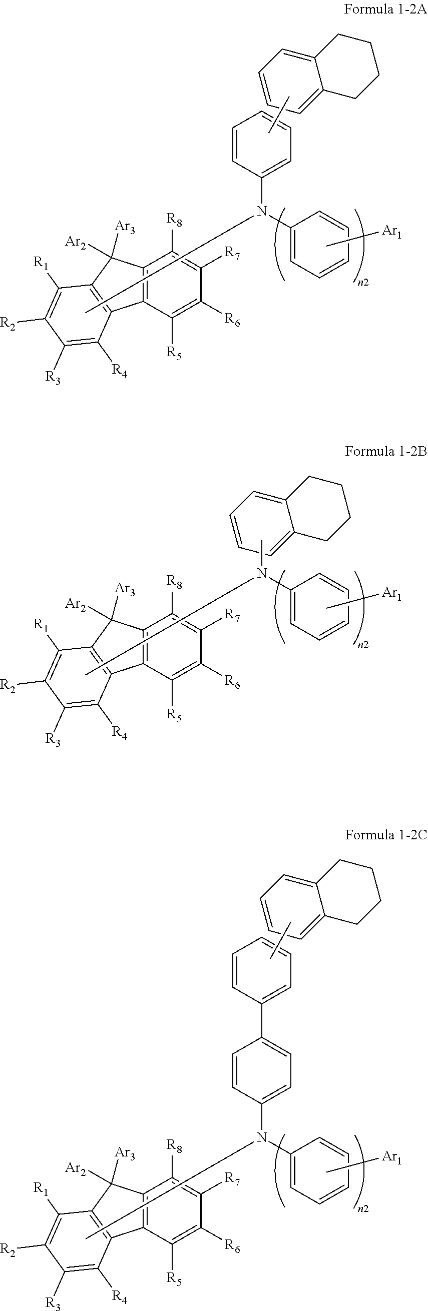

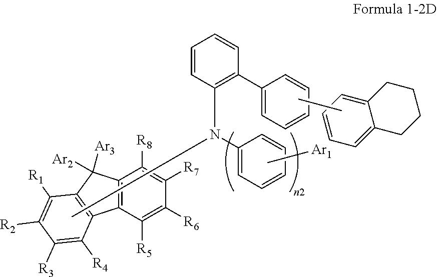

In Formula 1-2A to Formula 1-2D, the same descriptions as provided in Formula 1 may be applied for Ar₁ to Ar₃, n2, and R₁ to R₈.

Formula 1-2A represents Formula 1 where n1 is 1, L₁ is an unsubstituted phenylene group, and a tetrahydronaphthyl group is indirectly bonded to the nitrogen atom via the unsubstituted phenylene group. Formula 1-2B represents Formula 1 where n1 is 0, and a tetrahydronaphthyl group is directly bonded to the nitrogen atom.

Formula 1-2C and Formula 1-2D represent cases where n1 is 2, and L₁ is an unsubstituted phenylene group, and a tetrahydronaphthyl group is indirectly bonded to the nitrogen atom via two unsubstituted phenylene groups (that is, unsubstituted divalent biphenyl group). In Formula 1-2C, among two unsubstituted phenylene groups, one phenylene group is directly bonded to the nitrogen atom, and the remaining one phenylene group is bonded at the para position with respect to the nitrogen atom. In Formula 1-2D, among two unsubstituted phenylene groups, one phenylene group is directly bonded to the nitrogen atom, and the remaining one phenylene group is bonded at the ortho position with respect to the nitrogen atom.

In one or more embodiments, Formula 1-2A may be represented by any one of Formula 1-2AA or Formula 1-2AB. Formula 1-2AA and Formula 1-2AB represent Formula 1-2A where the bonding position of the tetrahydronaphthyl group is embodied.

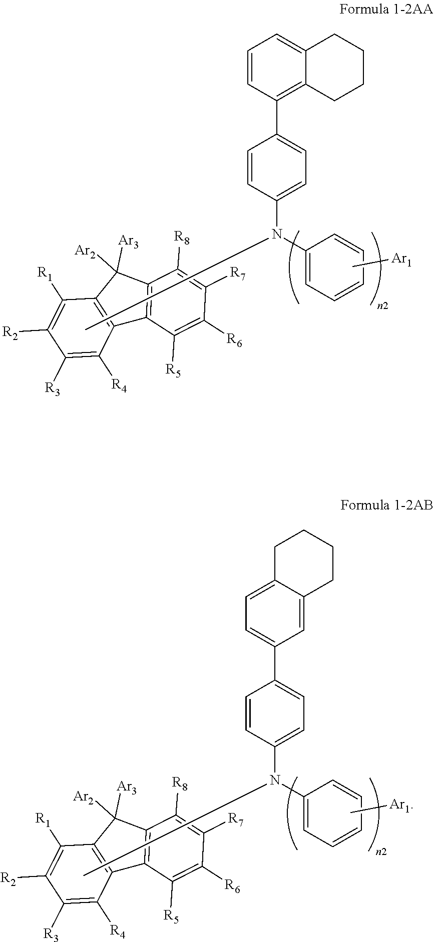

In Formula 1-2AA and Formula 1-2AB, the same descriptions as provided in Formula 1-2A may be applied for Ar₁ to Ar₃, n2, and R₁ to R₈.

Formula 1-2C may be represented by Formula 1-2CA or Formula 1-2CB. Formula 1-2CA and Formula 1-2CB represent Formula 1-2C where the bonding position of the tetrahydronaphthyl group is embodied.

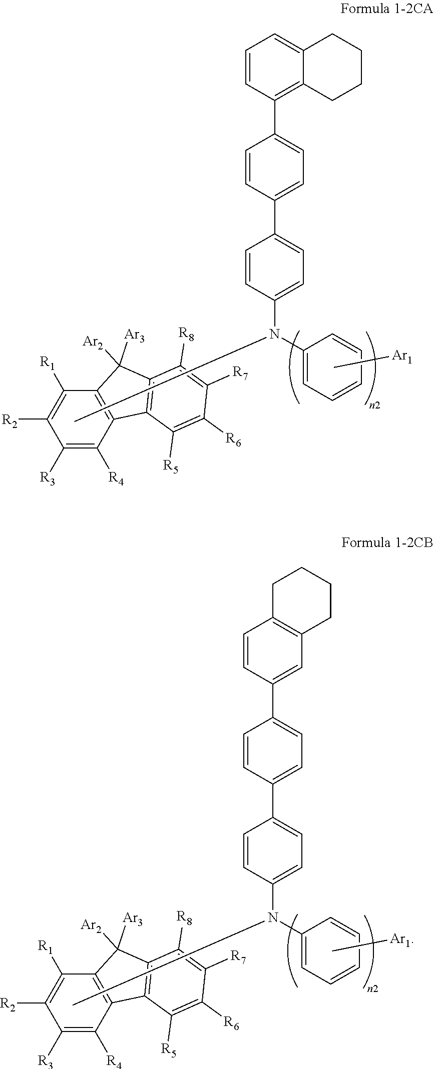

In Formula 1-2CA and Formula 1-2CB, the same descriptions as provided in Formula 1-2C may be applied for Ar₁ to Ar₃, n2, and R₁ to R₈.

Formula 1-2D may be represented by Formula 1-2DA or Formula 1-2DB.

Formula 1-2DA and Formula 1-2DB represent Formula 1-2D where the bonding position of the tetrahydronaphthyl group is embodied.

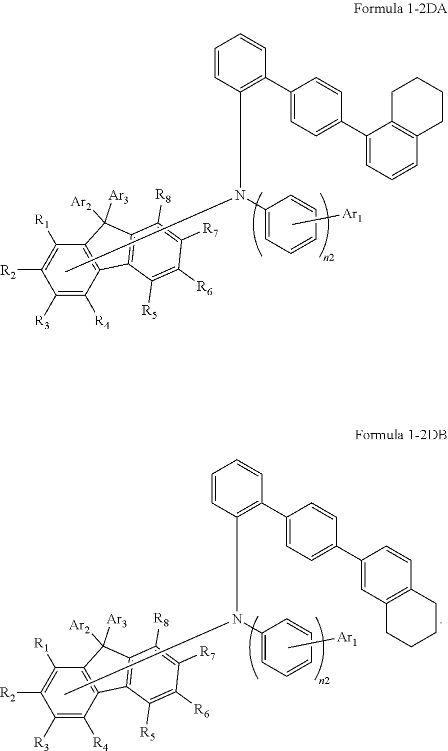

In Formula 1-2DA and Formula 1-2DB, the same descriptions as provided in Formula 1-2D may be applied for Ar₁ to Ar₃, n2, and R₁ to R₈.

In one or more embodiments, Formula 1 may be represented by any one of Formula 1-3A to Formula 1-3C. Formula 1-3A to Formula 1-3C represent Formula 1 where the bonding position of a fluorene moiety including R₁ to R₈ with respect to the nitrogen atom, Ar₂, and Ar₃ are embodied.

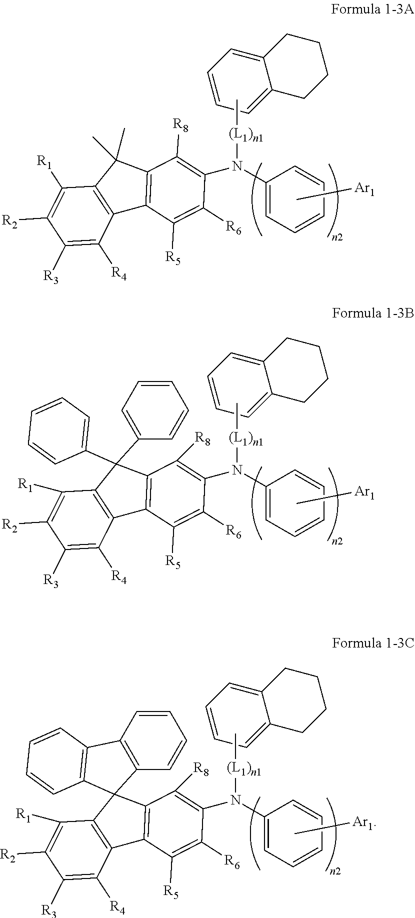

Formula 1-3A represents a case of Formula 1 where Ar₂ and Ar₃ are unsubstituted methyl groups. Formula 1-3B represents a case of Formula 1 where Ar₂ and Ar₃ are unsubstituted phenyl groups. Formula 1-3C represents a case of Formula 1 where Ar₂ and Ar₃ are unsubstituted phenyl groups and are combined with each other to form a spiro fluorenyl group.

In Formula 1-3A to Formula 1-3C, the same descriptions as provided in Formula 1 may be applied for Ar₁, L₁, n1, n2, and R₁ to R₈.

In one or more embodiments, Formula 1-3A may be represented by any one of Formula 1-3AA to Formula 1-3AC. Formula 1-3AA to Formula 1-3AC represent cases of Formula 1-3A where adjacent two among R₁ to R₈ are combined with each other to form a ring. In Formula 1-3A, because adjacent two among R₁ to R₈ are combined with each other to form a ring, a fused ring of four rings, including a fluorene moiety may be formed.

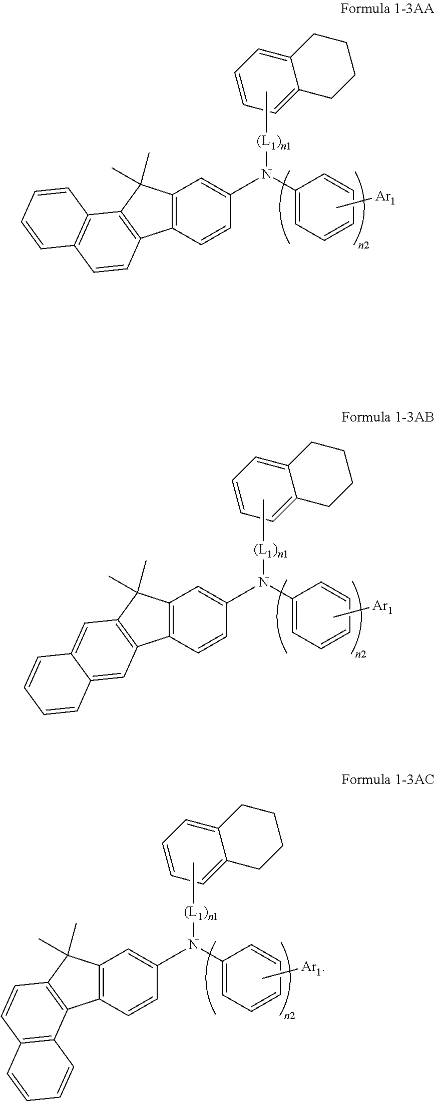

Formula 1-3AA represents a case of Formula 1-3A where R₁ and R₂ are combined to form a ring. Formula 1-3AB represents a case of Formula 1-3A where R₂ and R₃ are combined to form a ring. Formula 1-3AC represents a case of Formula 1-3A where R₃ and R₄ are combined to form a ring. In Formula 1-3AA to Formula 1-3AC, the same descriptions as provided in FIG. 1-3A may be applied for Ar₁, L₁, n1, and n2.

Formula 1-3A may be represented by Formula 1-3AD. Formula 1-3AD represents a case of Formula 1-3A where R₁, R₂, R₄, R₅, and R₈ are hydrogen atoms.

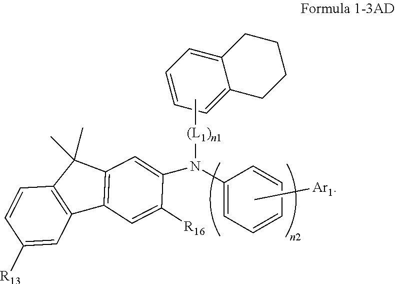

In Formula 1-3AD, R₁₃ and R₁₆ may be each independently a hydrogen atom, a substituted or unsubstituted methyl group, or a substituted or unsubstituted phenyl group. For example, any one of R₁₃ and/or R₁₆ may be an unsubstituted methyl group or an unsubstituted phenyl group. In Formula 1-3AD, the same descriptions as provided in Formula 1-3A may be applied for Ar₁, L₁, n1 and n2.

For example, Formula 1-3B may be represented by any one of Formula 3-BA to Formula 1-3BC. Formula 1-3BA represents a case of Formula 1-3B where R₁ and R₂ are combined to form a ring. Formula 1-3BB represents a case of Formula 1-3B where R₃ and R₄ are combined to form a ring. Formula 1-3BA and Formula 1-3BB are cases where a fused ring of four rings, including a fluorene moiety is formed. Formula 1-3BC represents a case of Formula 1-3B where R₁ to R₃, R₅, R₆, and R₈ are hydrogen atoms.

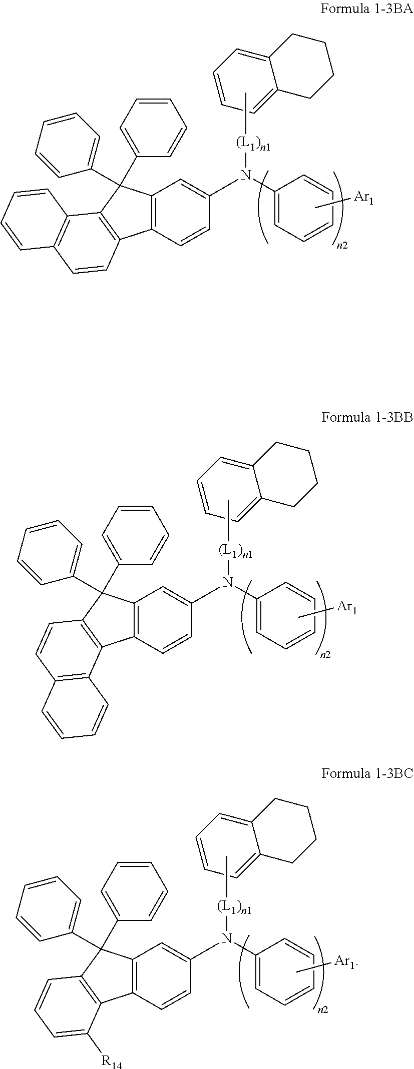

In Formula 1-3BC, R₁₄ may be a hydrogen atom or a substituted or unsubstituted phenyl group. In Formula 1-3BA to Formula 1-3BC, the same descriptions as provided in Formula 1-3B may be applied for Ar₁, n1, n2 and L₁.

For example, Formula 1-3C may be represented by any one of Formula 3-CA to Formula 1-3CC. Formula 1-3CA represents a case of Formula 1-3C where R₁ and R₂ are combined to form a ring. Formula 1-3CB represents a case of Formula 1-3C where R₃ and R₄ are combined to form a ring. Formula 1-3CA and Formula 1-3CB are cases where a fused ring of four rings, including a fluorene moiety is formed. Formula 1-3CC represents a case of Formula 1-3C where R₁ to R₃, R₅, R₆, and R₈ are hydrogen atoms.

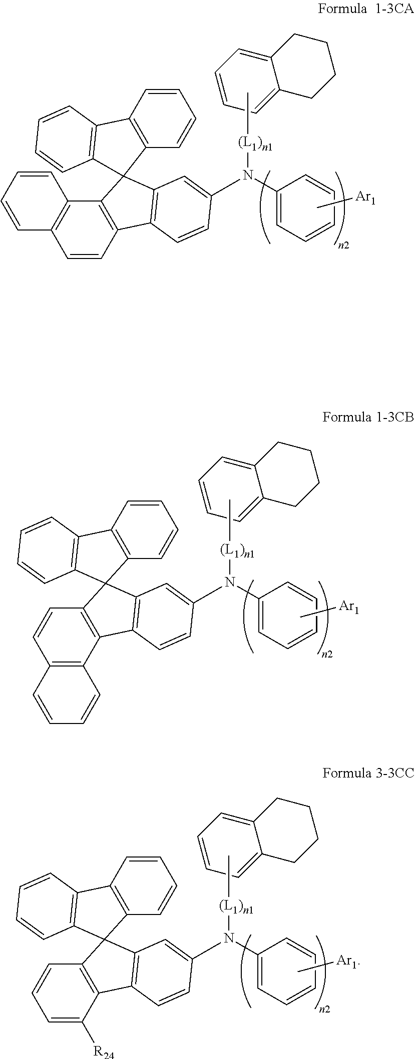

In Formula 1-3CC, R₂₄ may be a hydrogen atom or a substituted or unsubstituted phenyl group. In Formula 1-3CA to Formula 1-3CC, the same descriptions as provided in Formula 1-3C may be applied for Ar₁, n1, n2 and L₁.

The amine compound of one or more embodiments may be represented by any one of the compounds in Compound Group 1 below. The light emitting element ED of one or more embodiments may include at least one of the compounds in Compound Group 1.

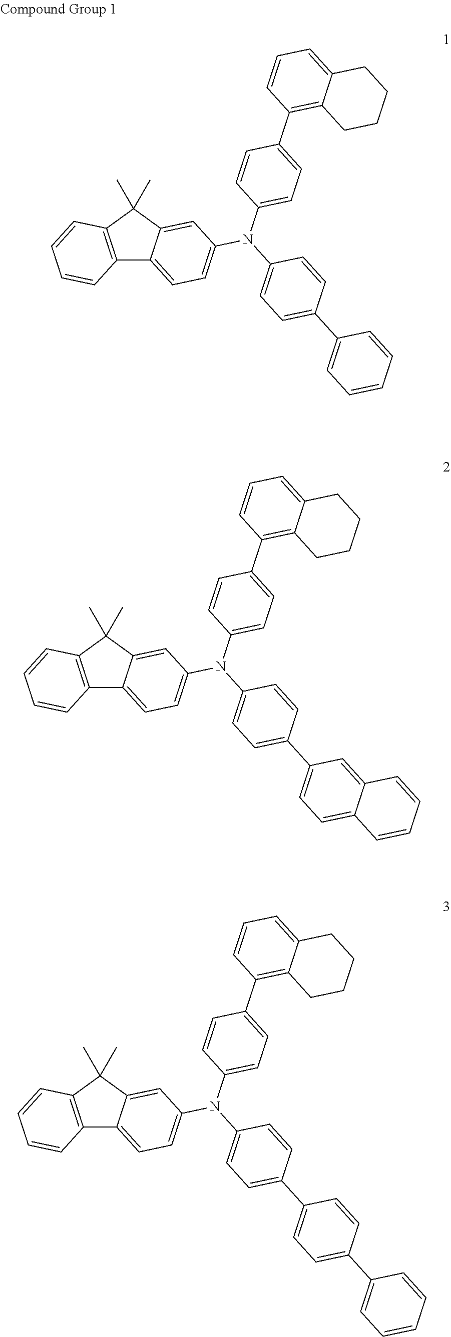 [SMILES: CC1(C)c2ccccc2-c2ccc(N(c3ccc(-c4ccccc4)cc3)c3ccc(-c4cccc5c4CCCC5)cc3)cc21 ; CC1(C)c2ccccc2-c2ccc(N(c3ccc(-c4ccc5ccccc5c4)cc3)c3ccc(-c4cccc5c4CCCC5)cc3)cc21 ; CC1(C)c2ccccc2-c2ccc(N(c3ccc(-c4ccc(-c5ccccc5)cc4)cc3)c3ccc(-c4cccc5c4CCCC5)cc3)cc21]

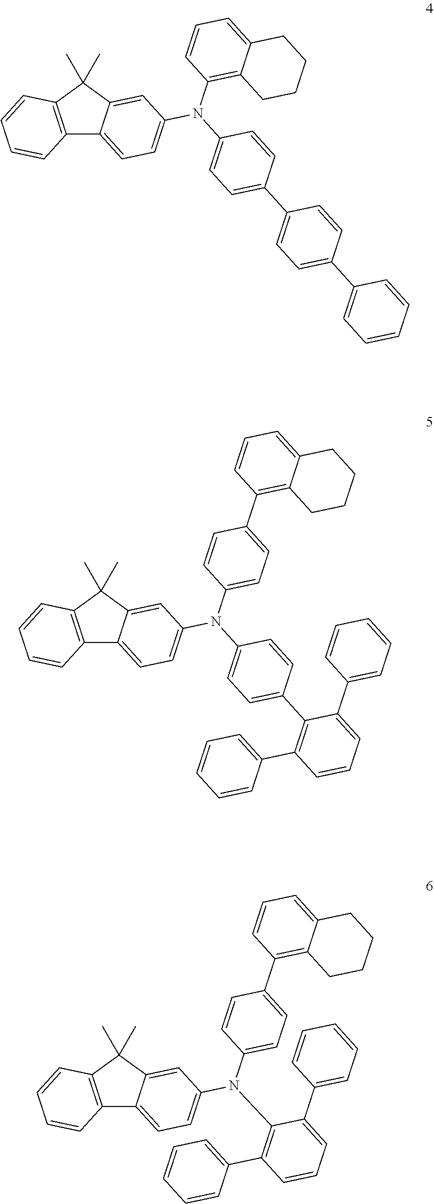 [SMILES: CC1(C)c2ccccc2-c2ccc(N(c3ccc(-c4ccc(-c5ccccc5)cc4)cc3)c3cccc4c3CCCC4)cc21 ; CC1(C)c2ccccc2-c2ccc(N(c3ccc(-c4cccc5c4CCCC5)cc3)c3ccc(-c4c(-c5ccccc5)cccc4-c4ccccc4)cc3)cc21 ; CC1(C)c2ccccc2-c2ccc(N(c3ccc(-c4cccc5c4CCCC5)cc3)c3c(-c4ccccc4)cccc3-c3ccccc3)cc21]

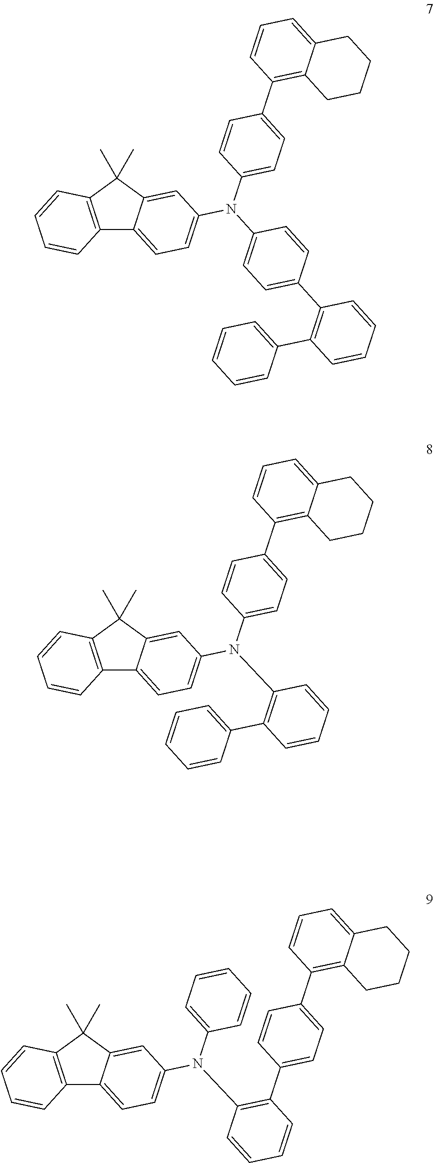 [SMILES: CC1(C)c2ccccc2-c2ccc(N(c3ccc(-c4ccccc4-c4ccccc4)cc3)c3ccc(-c4cccc5c4CCCC5)cc3)cc21 ; CC1(C)c2ccccc2-c2ccc(N(c3ccc(-c4cccc5c4CCCC5)cc3)c3ccccc3-c3ccccc3)cc21 ; CC1(C)c2ccccc2-c2ccc(N(c3ccccc3)c3ccccc3-c3ccc(-c4cccc5c4CCCC5)cc3)cc21]

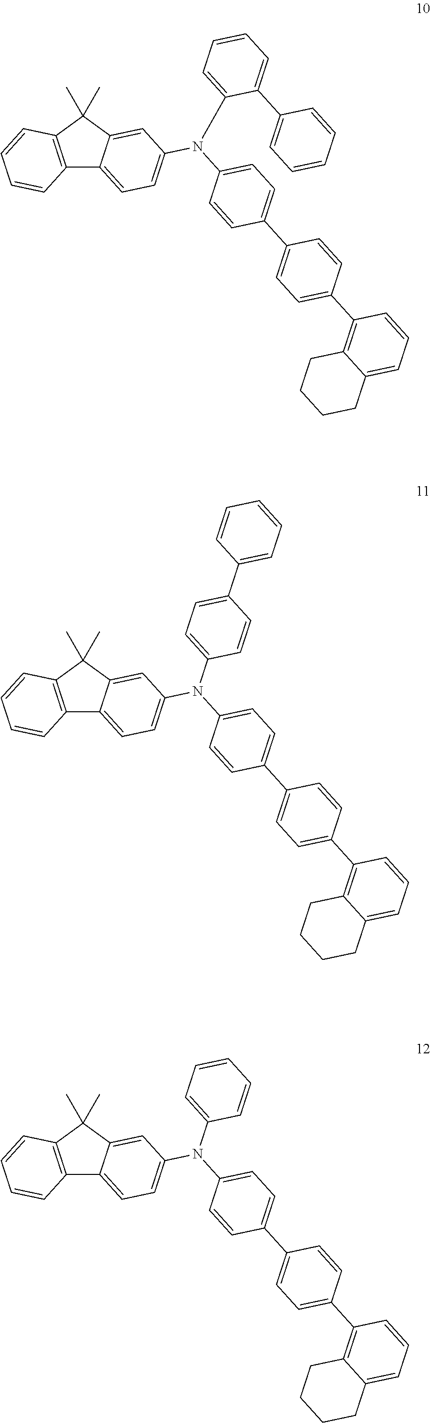 [SMILES: CC1(C)c2ccccc2-c2ccc(N(c3ccc(-c4ccc(-c5cccc6c5CCCC6)cc4)cc3)c3ccccc3-c3ccccc3)cc21 ; CC1(C)c2ccccc2-c2ccc(N(c3ccc(-c4ccccc4)cc3)c3ccc(-c4ccc(-c5cccc6c5CCCC6)cc4)cc3)cc21 ; CC1(C)c2ccccc2-c2ccc(N(c3ccccc3)c3ccc(-c4ccc(-c5cccc6c5CCCC6)cc4)cc3)cc21]

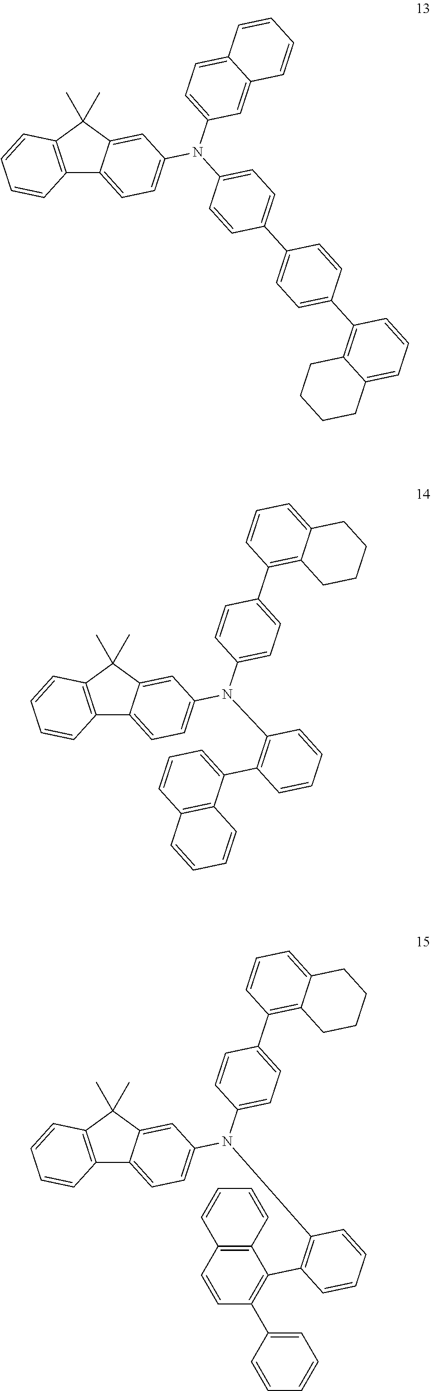 [SMILES: CC1(C)c2ccccc2-c2ccc(N(c3ccc(-c4ccc(-c5cccc6c5CCCC6)cc4)cc3)c3ccc4ccccc4c3)cc21 ; CC1(C)c2ccccc2-c2ccc(N(c3ccc(-c4cccc5c4CCCC5)cc3)c3ccccc3-c3cccc4ccccc34)cc21 ; CC1(C)c2ccccc2-c2ccc(N(c3ccc(-c4cccc5c4CCCC5)cc3)c3ccccc3-c3c(-c4ccccc4)ccc4ccccc34)cc21]

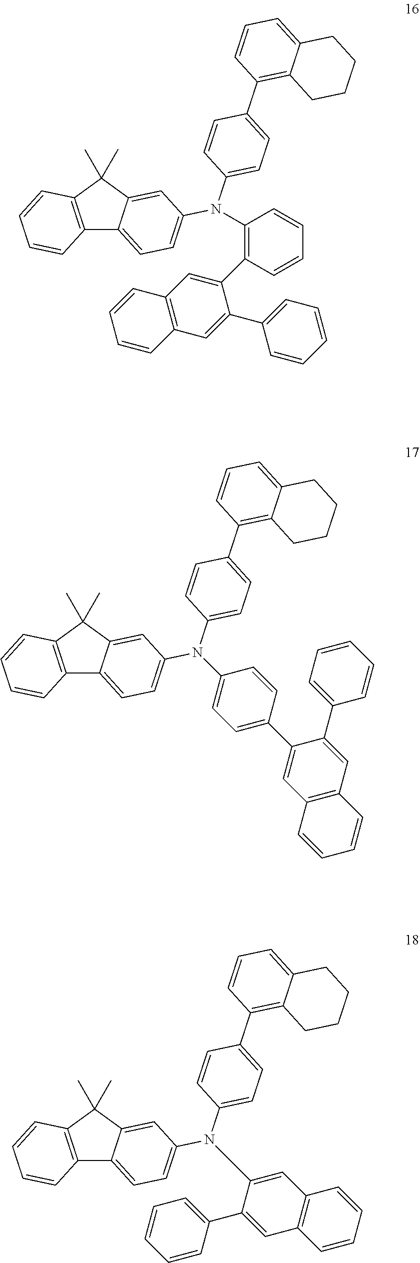 [SMILES: CC1(C)c2ccccc2-c2ccc(N(c3ccc(-c4cccc5c4CCCC5)cc3)c3ccccc3-c3cc4ccccc4cc3-c3ccccc3)cc21 ; CC1(C)c2ccccc2-c2ccc(N(c3ccc(-c4cc5ccccc5cc4-c4ccccc4)cc3)c3ccc(-c4cccc5c4CCCC5)cc3)cc21 ; CC1(C)c2ccccc2-c2ccc(N(c3ccc(-c4cccc5c4CCCC5)cc3)c3cc4ccccc4cc3-c3ccccc3)cc21]

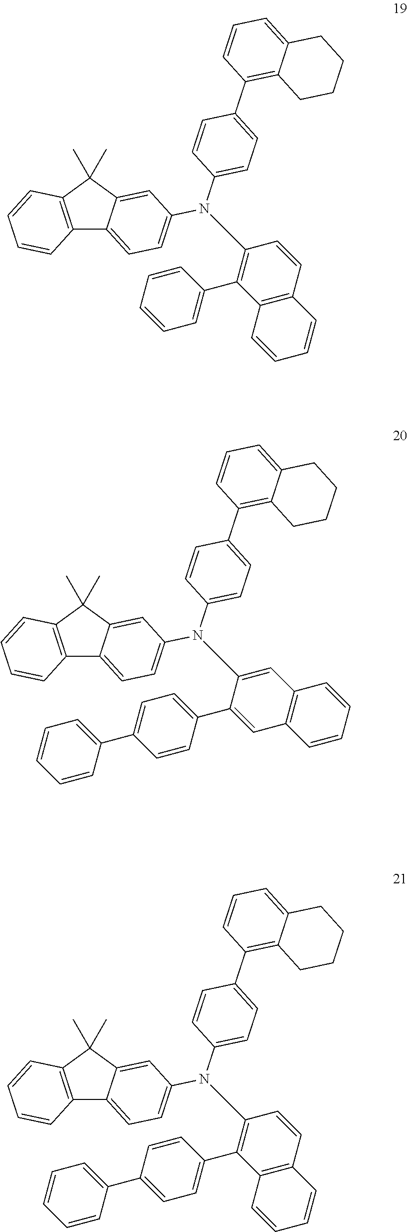 [SMILES: CC1(C)c2ccccc2-c2ccc(N(c3ccc(-c4cccc5c4CCCC5)cc3)c3ccc4ccccc4c3-c3ccccc3)cc21 ; CC1(C)c2ccccc2-c2ccc(N(c3ccc(-c4cccc5c4CCCC5)cc3)c3cc4ccccc4cc3-c3ccc(-c4ccccc4)cc3)cc21 ; CC1(C)c2ccccc2-c2ccc(N(c3ccc(-c4cccc5c4CCCC5)cc3)c3ccc4ccccc4c3-c3ccc(-c4ccccc4)cc3)cc21]

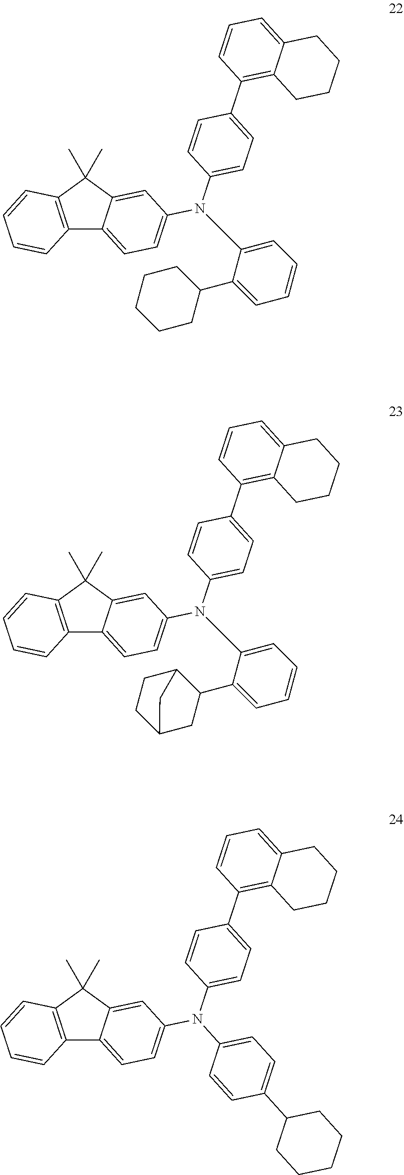 [SMILES: CC1(C)c2ccccc2-c2ccc(N(c3ccc(-c4cccc5c4CCCC5)cc3)c3ccccc3C3CCCCC3)cc21 ; CC1(C)c2ccccc2-c2ccc(N(c3ccc(-c4cccc5c4CCCC5)cc3)c3ccccc3C3CC4CCC3C4)cc21 ; CC1(C)c2ccccc2-c2ccc(N(c3ccc(-c4cccc5c4CCCC5)cc3)c3ccc(C4CCCCC4)cc3)cc21]

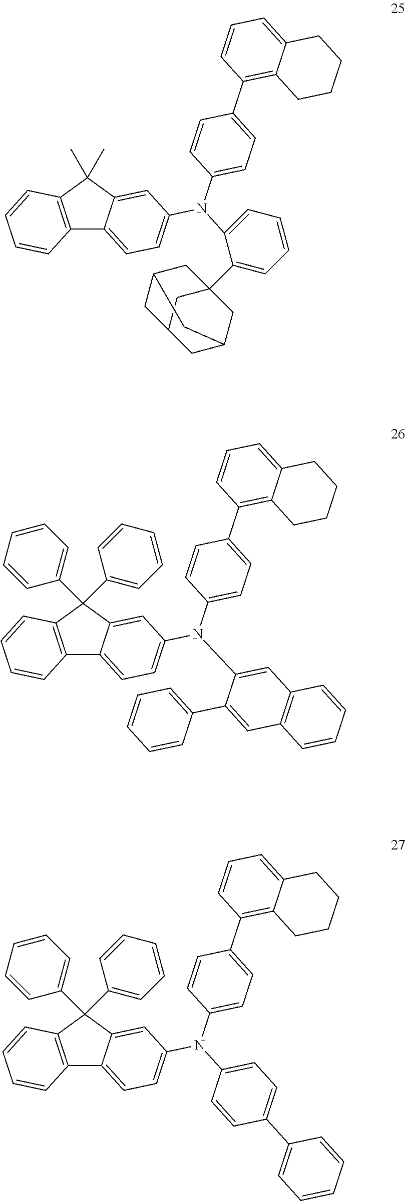 [SMILES: CC1(C)c2ccccc2-c2ccc(N(c3ccc(-c4cccc5c4CCCC5)cc3)c3ccccc3C34CC5CC(CC(C5)C3)C4)cc21 ; c1ccc(-c2cc3ccccc3cc2N(c2ccc(-c3cccc4c3CCCC4)cc2)c2ccc3c(c2)C(c2ccccc2)(c2ccccc2)c2ccccc2-3)cc1 ; c1ccc(-c2ccc(N(c3ccc(-c4cccc5c4CCCC5)cc3)c3ccc4c(c3)C(c3ccccc3)(c3ccccc3)c3ccccc3-4)cc2)cc1]

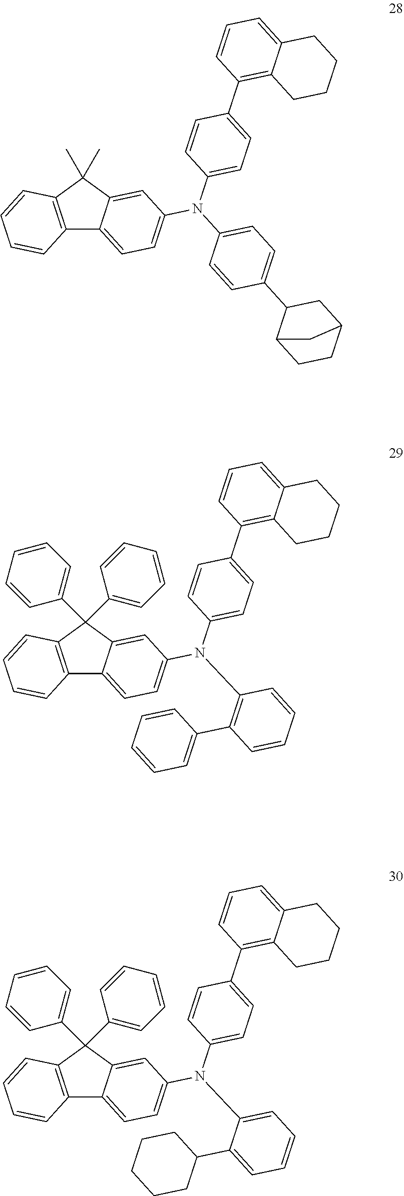 [SMILES: CC1(C)c2ccccc2-c2ccc(N(c3ccc(-c4cccc5c4CCCC5)cc3)c3ccc(C4CC5CCC4C5)cc3)cc21 ; c1ccc(-c2ccccc2N(c2ccc(-c3cccc4c3CCCC4)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5cccc6c5CCCC6)cc4)c4ccccc4C4CCCCC4)cc32)cc1]

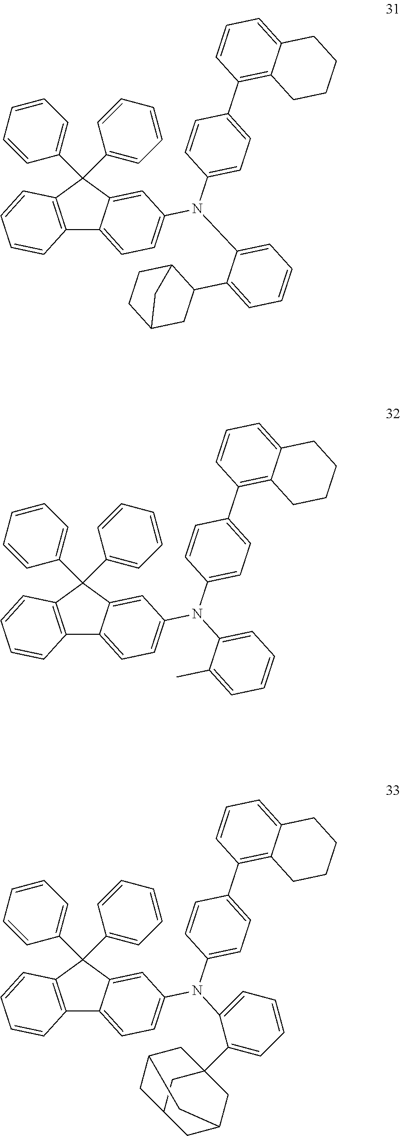 [SMILES: c1ccc(C2(c3ccccc3)c3ccccc3-c3ccc(N(c4ccc(-c5cccc6c5CCCC6)cc4)c4ccccc4C4CC5CCC4C5)cc32)cc1 ; Cc1ccccc1N(c1ccc(-c2cccc3c2CCCC3)cc1)c1ccc2c(c1)C(c1ccccc1)(c1ccccc1)c1ccccc1-2 ; c1ccc(C2(c3ccccc3)c3ccccc3-c3ccc(N(c4ccc(-c5cccc6c5CCCC6)cc4)c4ccccc4C45CC6CC(CC(C6)C4)C5)cc32)cc1]

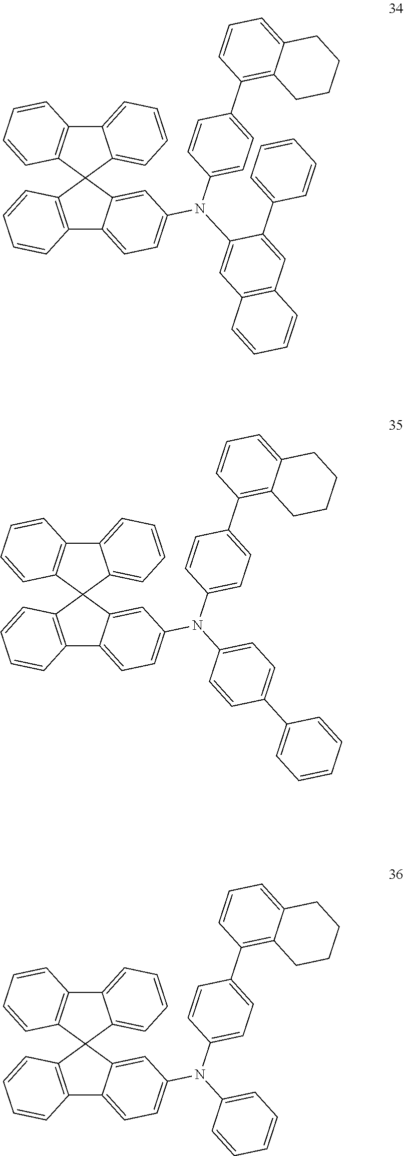 [SMILES: c1ccc(-c2cc3ccccc3cc2N(c2ccc(-c3cccc4c3CCCC4)cc2)c2ccc3c(c2)C2(c4ccccc4-c4ccccc42)c2ccccc2-3)cc1 ; c1ccc(-c2ccc(N(c3ccc(-c4cccc5c4CCCC5)cc3)c3ccc4c(c3)C3(c5ccccc5-c5ccccc53)c3ccccc3-4)cc2)cc1 ; c1ccc(N(c2ccc(-c3cccc4c3CCCC4)cc2)c2ccc3c(c2)C2(c4ccccc4-c4ccccc42)c2ccccc2-3)cc1]

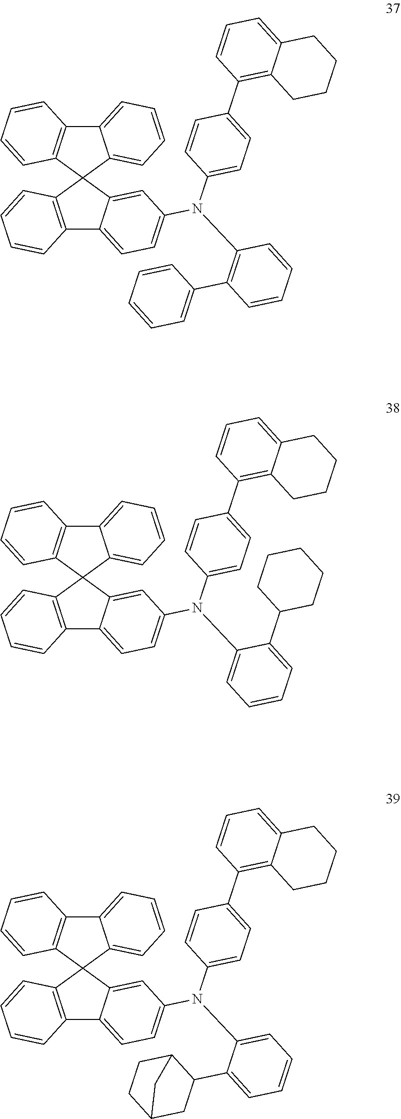 [SMILES: c1ccc(-c2ccccc2N(c2ccc(-c3cccc4c3CCCC4)cc2)c2ccc3c(c2)C2(c4ccccc4-c4ccccc42)c2ccccc2-3)cc1 ; c1ccc2c(c1)-c1ccccc1C21c2ccccc2-c2ccc(N(c3ccc(-c4cccc5c4CCCC5)cc3)c3ccccc3C3CCCCC3)cc21 ; c1ccc2c(c1)-c1ccccc1C21c2ccccc2-c2ccc(N(c3ccc(-c4cccc5c4CCCC5)cc3)c3ccccc3C3CC4CCC3C4)cc21]

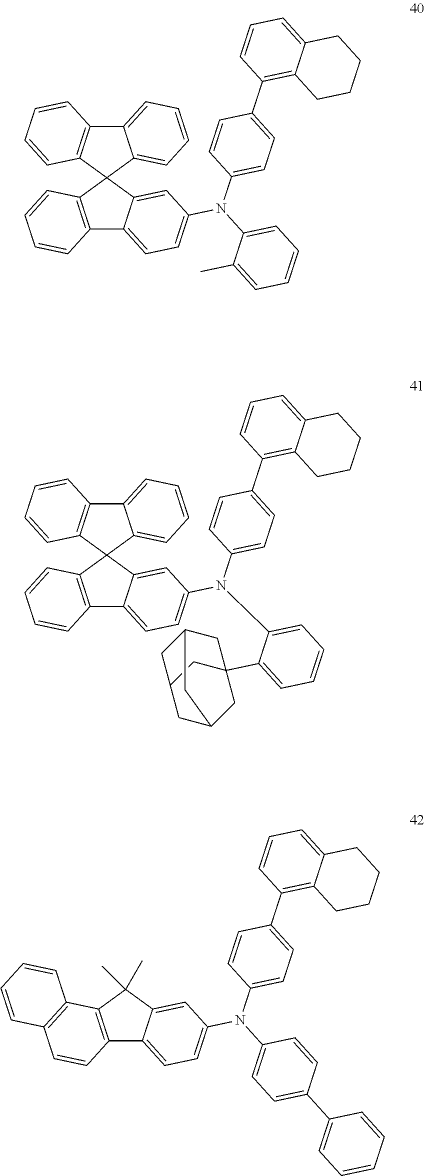 [SMILES: Cc1ccccc1N(c1ccc(-c2cccc3c2CCCC3)cc1)c1ccc2c(c1)C1(c3ccccc3-c3ccccc31)c1ccccc1-2 ; c1ccc2c(c1)-c1ccccc1C21c2ccccc2-c2ccc(N(c3ccc(-c4cccc5c4CCCC5)cc3)c3ccccc3C34CC5CC(CC(C5)C3)C4)cc21 ; CC1(C)c2cc(N(c3ccc(-c4ccccc4)cc3)c3ccc(-c4cccc5c4CCCC5)cc3)ccc2-c2ccc3ccccc3c21]

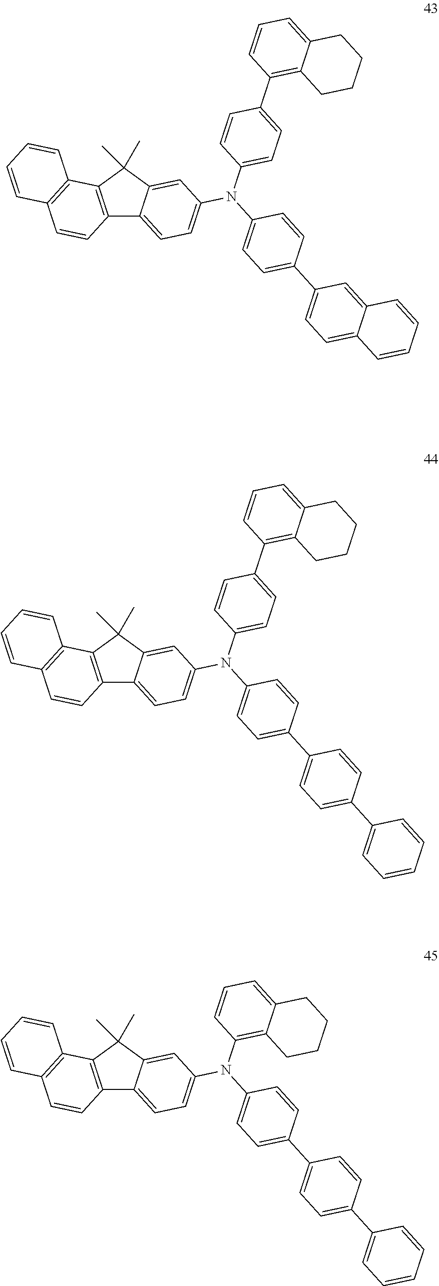 [SMILES: CC1(C)c2cc(N(c3ccc(-c4ccc5ccccc5c4)cc3)c3ccc(-c4cccc5c4CCCC5)cc3)ccc2-c2ccc3ccccc3c21 ; CC1(C)c2cc(N(c3ccc(-c4ccc(-c5ccccc5)cc4)cc3)c3ccc(-c4cccc5c4CCCC5)cc3)ccc2-c2ccc3ccccc3c21 ; CC1(C)c2cc(N(c3ccc(-c4ccc(-c5ccccc5)cc4)cc3)c3cccc4c3CCCC4)ccc2-c2ccc3ccccc3c21]

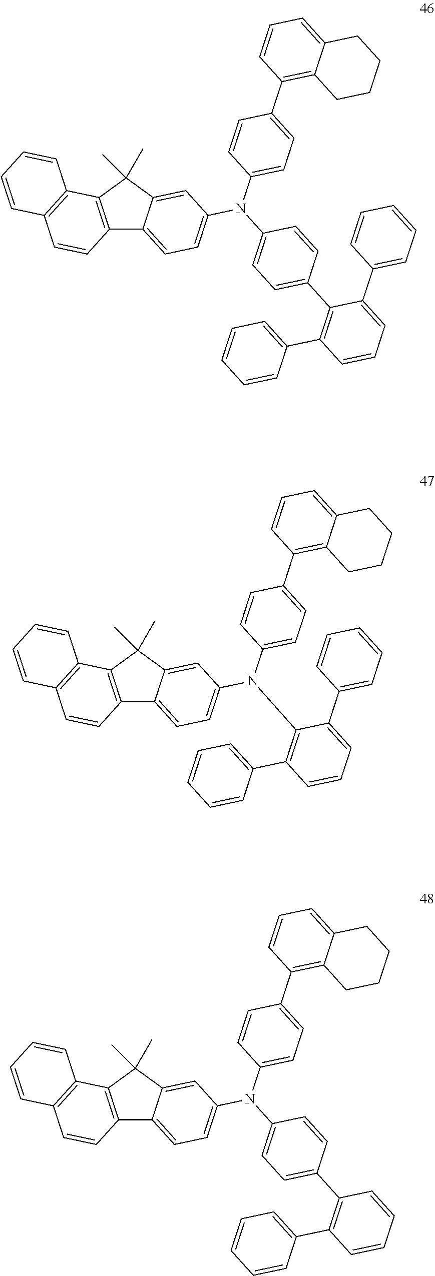 [SMILES: CC1(C)c2cc(N(c3ccc(-c4cccc5c4CCCC5)cc3)c3ccc(-c4c(-c5ccccc5)cccc4-c4ccccc4)cc3)ccc2-c2ccc3ccccc3c21 ; CC1(C)c2cc(N(c3ccc(-c4cccc5c4CCCC5)cc3)c3c(-c4ccccc4)cccc3-c3ccccc3)ccc2-c2ccc3ccccc3c21 ; CC1(C)c2cc(N(c3ccc(-c4ccccc4-c4ccccc4)cc3)c3ccc(-c4cccc5c4CCCC5)cc3)ccc2-c2ccc3ccccc3c21]

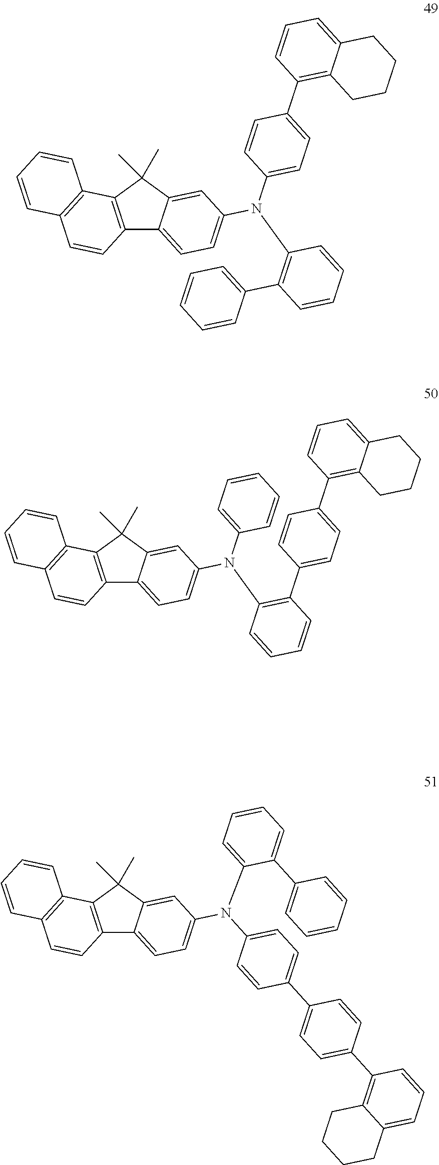 [SMILES: CC1(C)c2cc(N(c3ccc(-c4cccc5c4CCCC5)cc3)c3ccccc3-c3ccccc3)ccc2-c2ccc3ccccc3c21 ; CC1(C)c2cc(N(c3ccccc3)c3ccccc3-c3ccc(-c4cccc5c4CCCC5)cc3)ccc2-c2ccc3ccccc3c21 ; CC1(C)c2cc(N(c3ccc(-c4ccc(-c5cccc6c5CCCC6)cc4)cc3)c3ccccc3-c3ccccc3)ccc2-c2ccc3ccccc3c21]

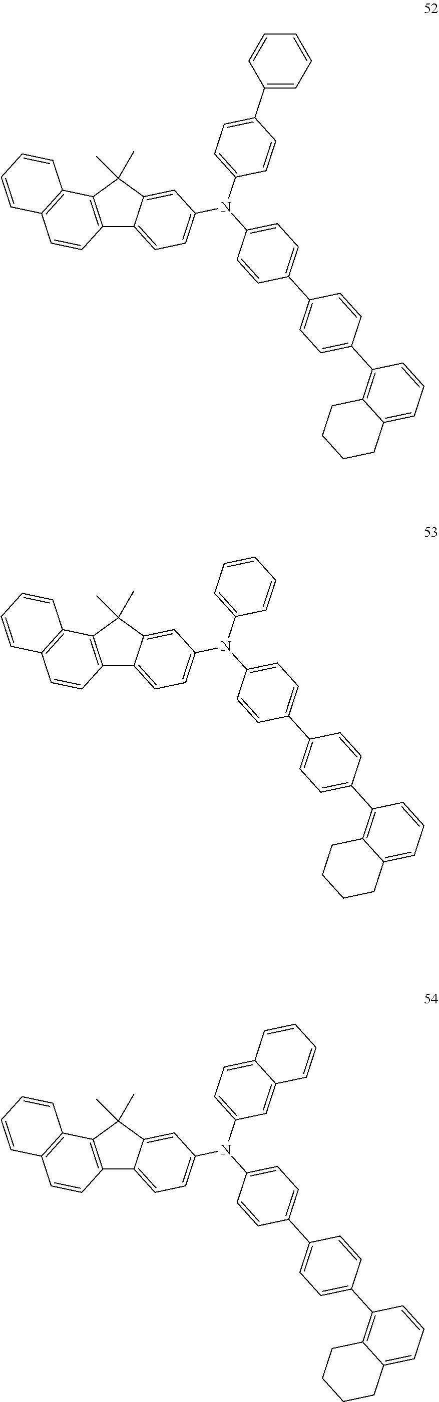 [SMILES: CC1(C)c2cc(N(c3ccc(-c4ccccc4)cc3)c3ccc(-c4ccc(-c5cccc6c5CCCC6)cc4)cc3)ccc2-c2ccc3ccccc3c21 ; CC1(C)c2cc(N(c3ccccc3)c3ccc(-c4ccc(-c5cccc6c5CCCC6)cc4)cc3)ccc2-c2ccc3ccccc3c21 ; CC1(C)c2cc(N(c3ccc(-c4ccc(-c5cccc6c5CCCC6)cc4)cc3)c3ccc4ccccc4c3)ccc2-c2ccc3ccccc3c21]

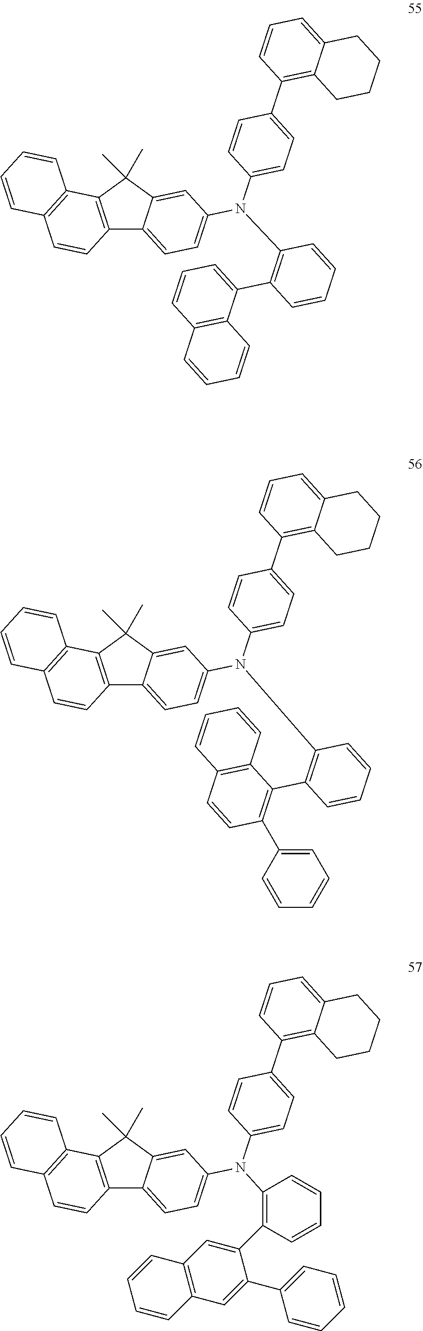 [SMILES: CC1(C)c2cc(N(c3ccc(-c4cccc5c4CCCC5)cc3)c3ccccc3-c3cccc4ccccc34)ccc2-c2ccc3ccccc3c21 ; CC1(C)c2cc(N(c3ccc(-c4cccc5c4CCCC5)cc3)c3ccccc3-c3c(-c4ccccc4)ccc4ccccc34)ccc2-c2ccc3ccccc3c21 ; CC1(C)c2cc(N(c3ccc(-c4cccc5c4CCCC5)cc3)c3ccccc3-c3cc4ccccc4cc3-c3ccccc3)ccc2-c2ccc3ccccc3c21]

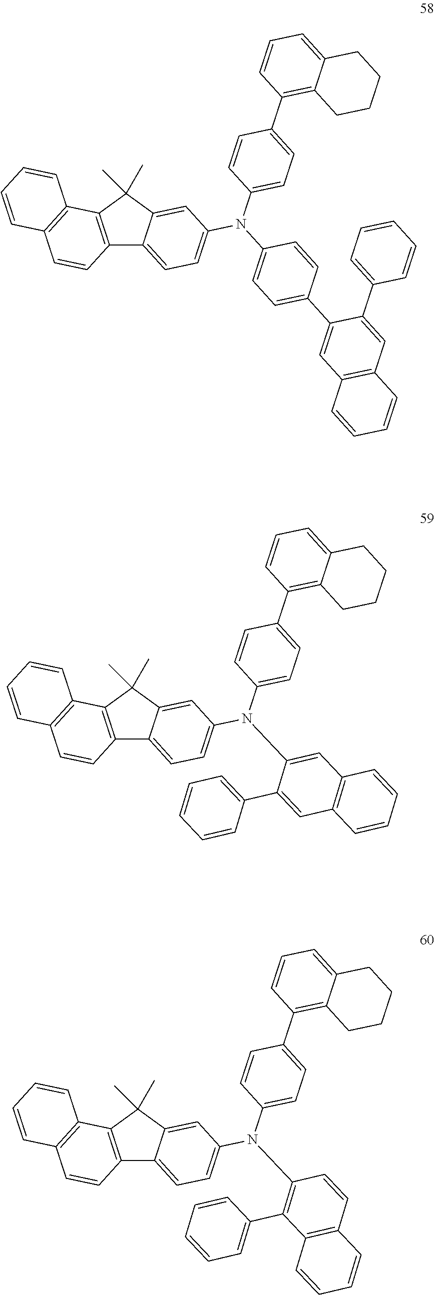 [SMILES: CC1(C)c2cc(N(c3ccc(-c4cc5ccccc5cc4-c4ccccc4)cc3)c3ccc(-c4cccc5c4CCCC5)cc3)ccc2-c2ccc3ccccc3c21 ; CC1(C)c2cc(N(c3ccc(-c4cccc5c4CCCC5)cc3)c3cc4ccccc4cc3-c3ccccc3)ccc2-c2ccc3ccccc3c21 ; CC1(C)c2cc(N(c3ccc(-c4cccc5c4CCCC5)cc3)c3ccc4ccccc4c3-c3ccccc3)ccc2-c2ccc3ccccc3c21]

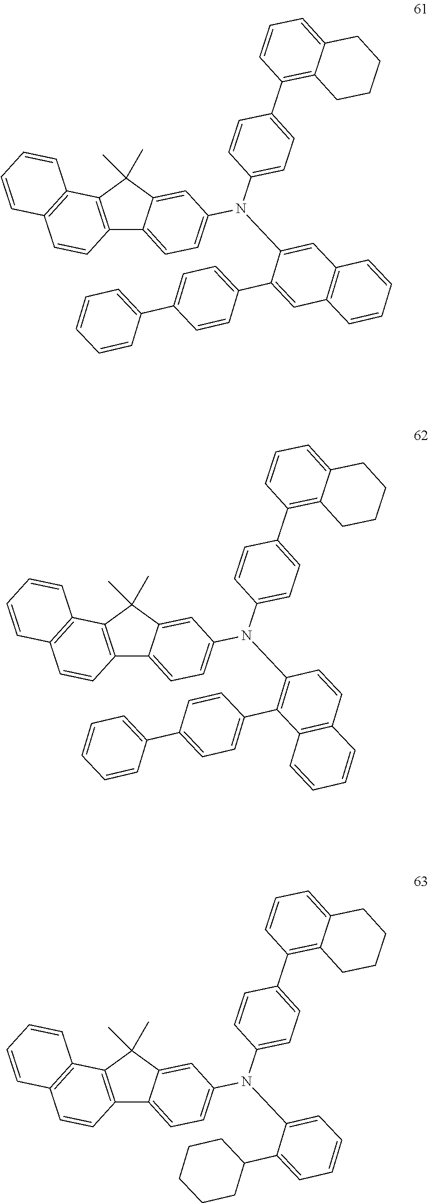 [SMILES: CC1(C)c2cc(N(c3ccc(-c4cccc5c4CCCC5)cc3)c3cc4ccccc4cc3-c3ccc(-c4ccccc4)cc3)ccc2-c2ccc3ccccc3c21 ; CC1(C)c2cc(N(c3ccc(-c4cccc5c4CCCC5)cc3)c3ccc4ccccc4c3-c3ccc(-c4ccccc4)cc3)ccc2-c2ccc3ccccc3c21 ; CC1(C)c2cc(N(c3ccc(-c4cccc5c4CCCC5)cc3)c3ccccc3C3CCCCC3)ccc2-c2ccc3ccccc3c21]

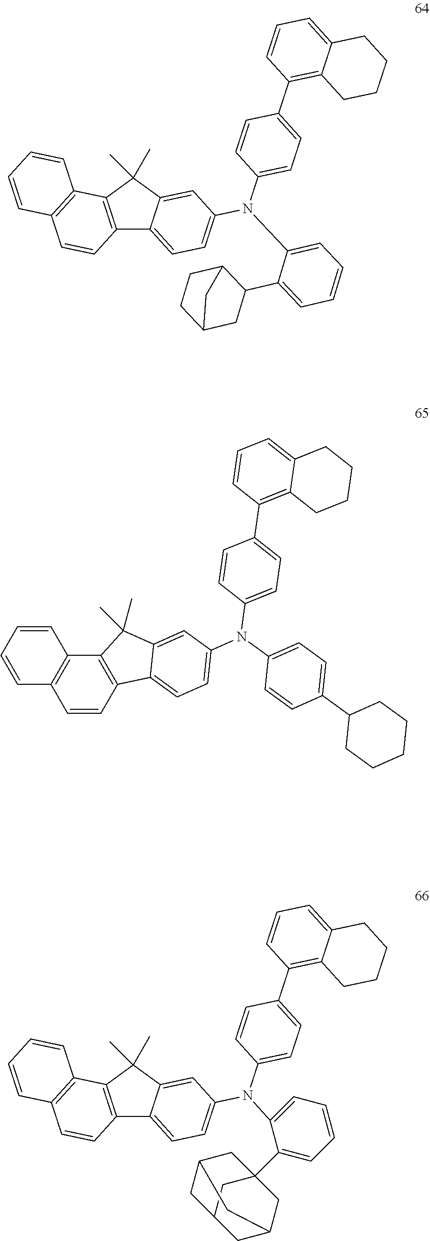 [SMILES: CC1(C)c2cc(N(c3ccc(-c4cccc5c4CCCC5)cc3)c3ccccc3C3CC4CCC3C4)ccc2-c2ccc3ccccc3c21 ; CC1(C)c2cc(N(c3ccc(-c4cccc5c4CCCC5)cc3)c3ccc(C4CCCCC4)cc3)ccc2-c2ccc3ccccc3c21 ; CC1(C)c2cc(N(c3ccc(-c4cccc5c4CCCC5)cc3)c3ccccc3C34CC5CC(CC(C5)C3)C4)ccc2-c2ccc3ccccc3c21]

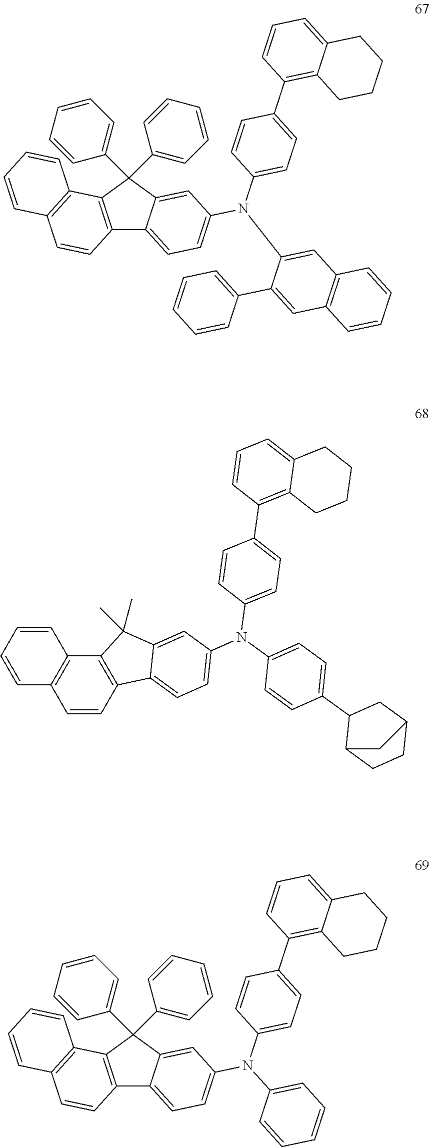 [SMILES: c1ccc(-c2cc3ccccc3cc2N(c2ccc(-c3cccc4c3CCCC4)cc2)c2ccc3c(c2)C(c2ccccc2)(c2ccccc2)c2c-3ccc3ccccc23)cc1 ; CC1(C)c2cc(N(c3ccc(-c4cccc5c4CCCC5)cc3)c3ccc(C4CC5CCC4C5)cc3)ccc2-c2ccc3ccccc3c21 ; c1ccc(N(c2ccc(-c3cccc4c3CCCC4)cc2)c2ccc3c(c2)C(c2ccccc2)(c2ccccc2)c2c-3ccc3ccccc23)cc1]

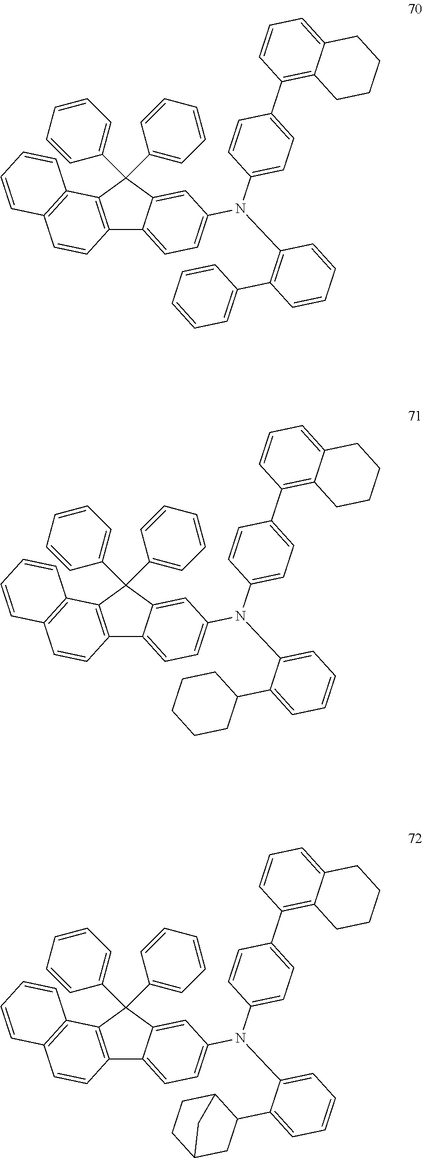 [SMILES: c1ccc(-c2ccccc2N(c2ccc(-c3cccc4c3CCCC4)cc2)c2ccc3c(c2)C(c2ccccc2)(c2ccccc2)c2c-3ccc3ccccc23)cc1 ; c1ccc(C2(c3ccccc3)c3cc(N(c4ccc(-c5cccc6c5CCCC6)cc4)c4ccccc4C4CCCCC4)ccc3-c3ccc4ccccc4c32)cc1 ; c1ccc(C2(c3ccccc3)c3cc(N(c4ccc(-c5cccc6c5CCCC6)cc4)c4ccccc4C4CC5CCC4C5)ccc3-c3ccc4ccccc4c32)cc1]

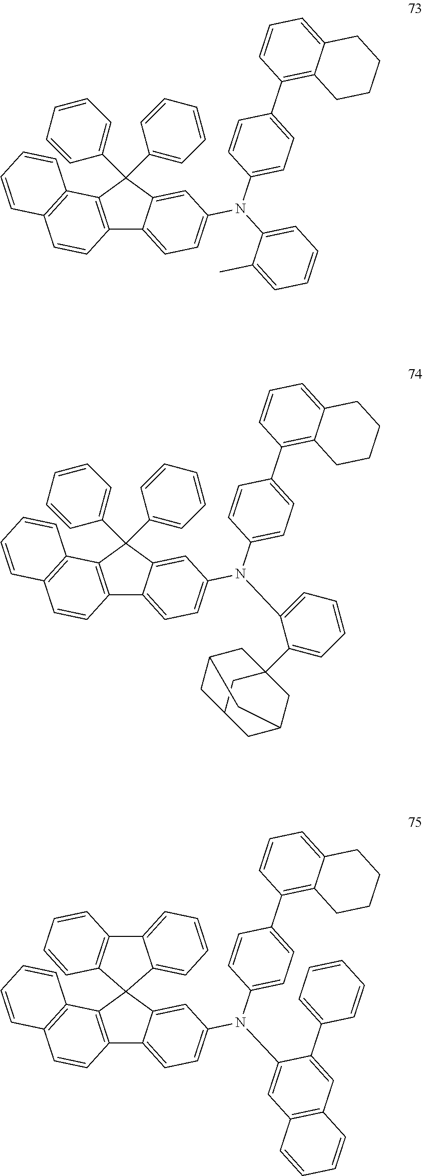 [SMILES: Cc1ccccc1N(c1ccc(-c2cccc3c2CCCC3)cc1)c1ccc2c(c1)C(c1ccccc1)(c1ccccc1)c1c-2ccc2ccccc12 ; c1ccc(C2(c3ccccc3)c3cc(N(c4ccc(-c5cccc6c5CCCC6)cc4)c4ccccc4C45CC6CC(CC(C6)C4)C5)ccc3-c3ccc4ccccc4c32)cc1 ; c1ccc(-c2cc3ccccc3cc2N(c2ccc(-c3cccc4c3CCCC4)cc2)c2ccc3c(c2)C2(c4ccccc4-c4ccccc42)c2c-3ccc3ccccc23)cc1]

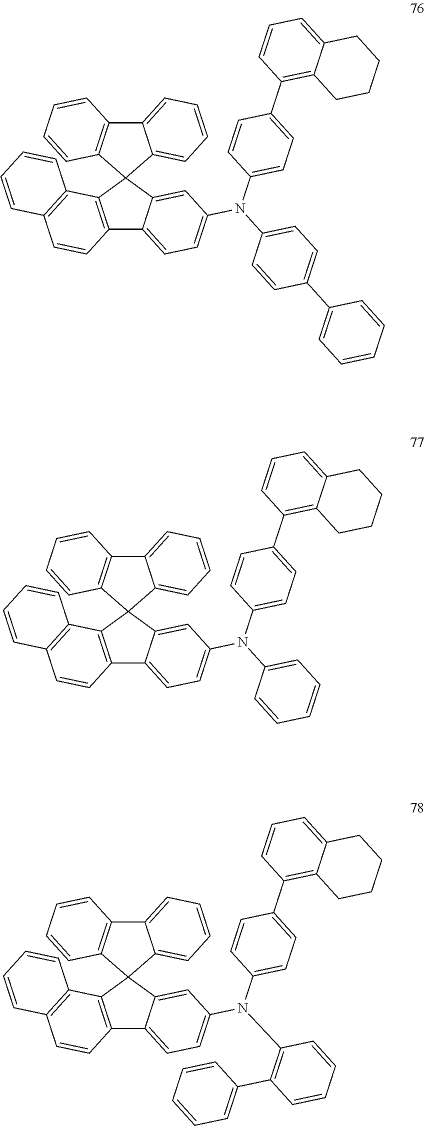 [SMILES: c1ccc(-c2ccc(N(c3ccc(-c4cccc5c4CCCC5)cc3)c3ccc4c(c3)C3(c5ccccc5-c5ccccc53)c3c-4ccc4ccccc34)cc2)cc1 ; c1ccc(N(c2ccc(-c3cccc4c3CCCC4)cc2)c2ccc3c(c2)C2(c4ccccc4-c4ccccc42)c2c-3ccc3ccccc23)cc1 ; c1ccc(-c2ccccc2N(c2ccc(-c3cccc4c3CCCC4)cc2)c2ccc3c(c2)C2(c4ccccc4-c4ccccc42)c2c-3ccc3ccccc23)cc1]

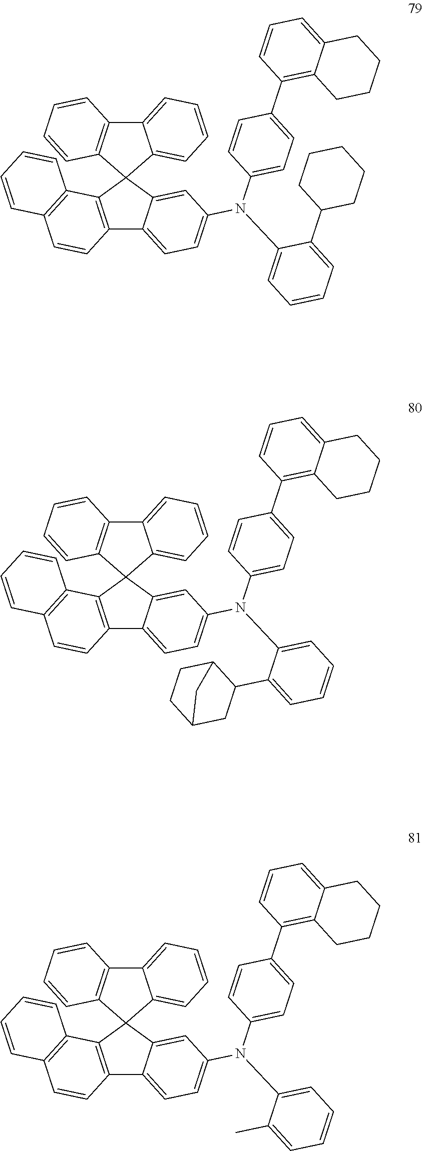 [SMILES: c1ccc2c(c1)-c1ccccc1C21c2cc(N(c3ccc(-c4cccc5c4CCCC5)cc3)c3ccccc3C3CCCCC3)ccc2-c2ccc3ccccc3c21 ; c1ccc2c(c1)-c1ccccc1C21c2cc(N(c3ccc(-c4cccc5c4CCCC5)cc3)c3ccccc3C3CC4CCC3C4)ccc2-c2ccc3ccccc3c21 ; Cc1ccccc1N(c1ccc(-c2cccc3c2CCCC3)cc1)c1ccc2c(c1)C1(c3ccccc3-c3ccccc31)c1c-2ccc2ccccc12]

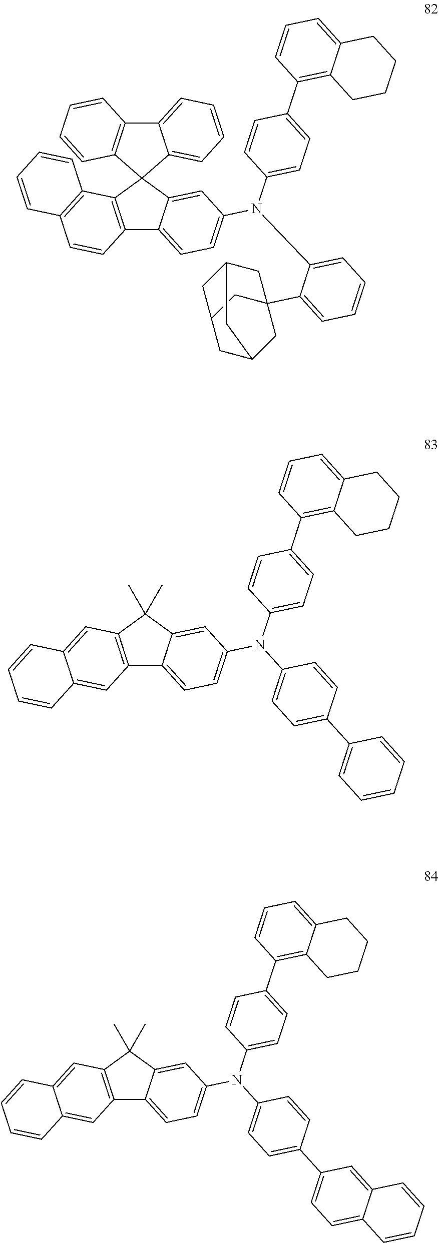 [SMILES: c1ccc2c(c1)-c1ccccc1C21c2cc(N(c3ccc(-c4cccc5c4CCCC5)cc3)c3ccccc3C34CC5CC(CC(C5)C3)C4)ccc2-c2ccc3ccccc3c21 ; CC1(C)c2cc(N(c3ccc(-c4ccccc4)cc3)c3ccc(-c4cccc5c4CCCC5)cc3)ccc2-c2cc3ccccc3cc21 ; CC1(C)c2cc(N(c3ccc(-c4ccc5ccccc5c4)cc3)c3ccc(-c4cccc5c4CCCC5)cc3)ccc2-c2cc3ccccc3cc21]

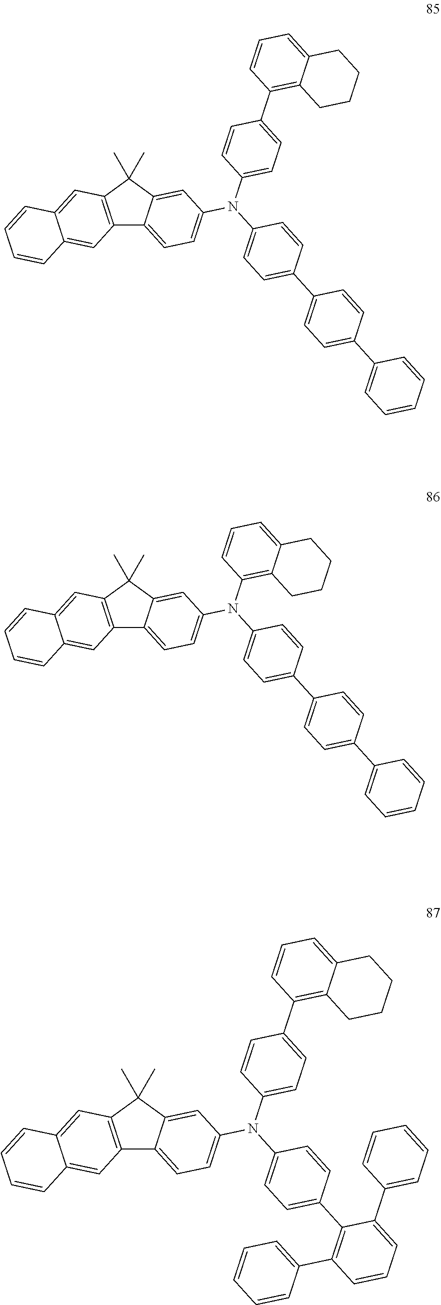 [SMILES: CC1(C)c2cc(N(c3ccc(-c4ccc(-c5ccccc5)cc4)cc3)c3ccc(-c4cccc5c4CCCC5)cc3)ccc2-c2cc3ccccc3cc21 ; CC1(C)c2cc(N(c3ccc(-c4ccc(-c5ccccc5)cc4)cc3)c3cccc4c3CCCC4)ccc2-c2cc3ccccc3cc21 ; CC1(C)c2cc(N(c3ccc(-c4cccc5c4CCCC5)cc3)c3ccc(-c4c(-c5ccccc5)cccc4-c4ccccc4)cc3)ccc2-c2cc3ccccc3cc21]

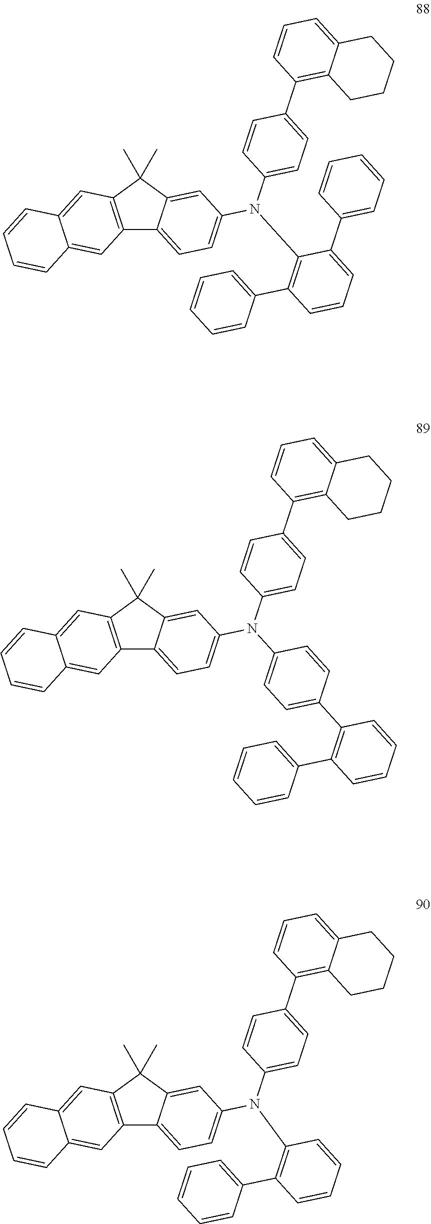 [SMILES: CC1(C)c2cc(N(c3ccc(-c4cccc5c4CCCC5)cc3)c3c(-c4ccccc4)cccc3-c3ccccc3)ccc2-c2cc3ccccc3cc21 ; CC1(C)c2cc(N(c3ccc(-c4ccccc4-c4ccccc4)cc3)c3ccc(-c4cccc5c4CCCC5)cc3)ccc2-c2cc3ccccc3cc21 ; CC1(C)c2cc(N(c3ccc(-c4cccc5c4CCCC5)cc3)c3ccccc3-c3ccccc3)ccc2-c2cc3ccccc3cc21]

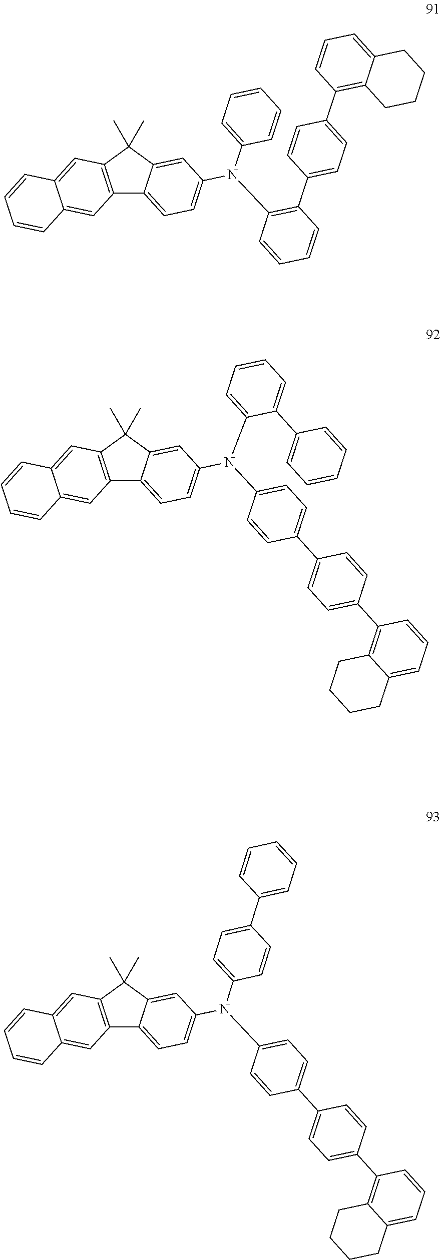 [SMILES: CC1(C)c2cc(N(c3ccccc3)c3ccccc3-c3ccc(-c4cccc5c4CCCC5)cc3)ccc2-c2cc3ccccc3cc21 ; CC1(C)c2cc(N(c3ccc(-c4ccc(-c5cccc6c5CCCC6)cc4)cc3)c3ccccc3-c3ccccc3)ccc2-c2cc3ccccc3cc21 ; CC1(C)c2cc(N(c3ccc(-c4ccccc4)cc3)c3ccc(-c4ccc(-c5cccc6c5CCCC6)cc4)cc3)ccc2-c2cc3ccccc3cc21]

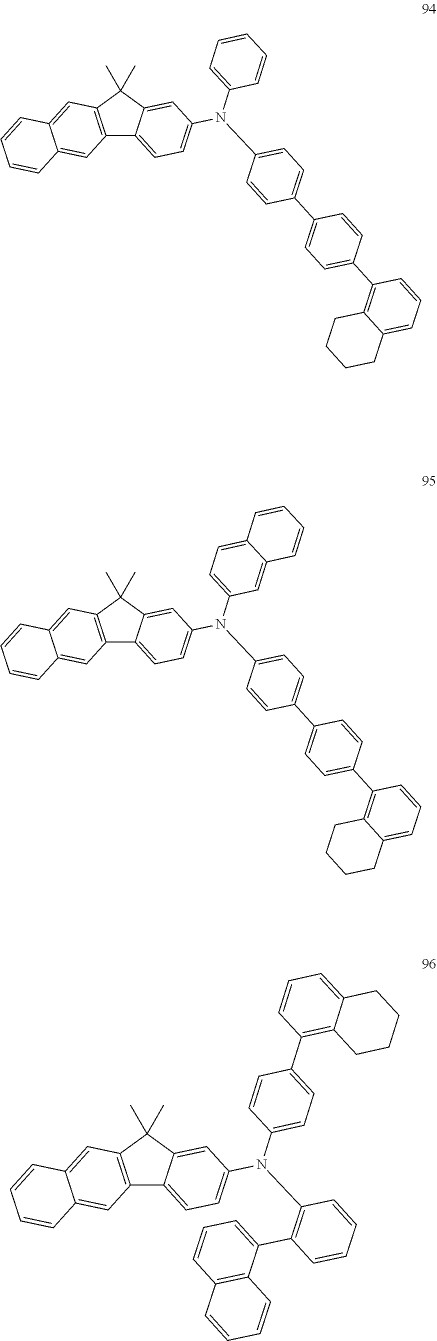 [SMILES: CC1(C)c2cc(N(c3ccccc3)c3ccc(-c4ccc(-c5cccc6c5CCCC6)cc4)cc3)ccc2-c2cc3ccccc3cc21 ; CC1(C)c2cc(N(c3ccc(-c4ccc(-c5cccc6c5CCCC6)cc4)cc3)c3ccc4ccccc4c3)ccc2-c2cc3ccccc3cc21 ; CC1(C)c2cc(N(c3ccc(-c4cccc5c4CCCC5)cc3)c3ccccc3-c3cccc4ccccc34)ccc2-c2cc3ccccc3cc21]

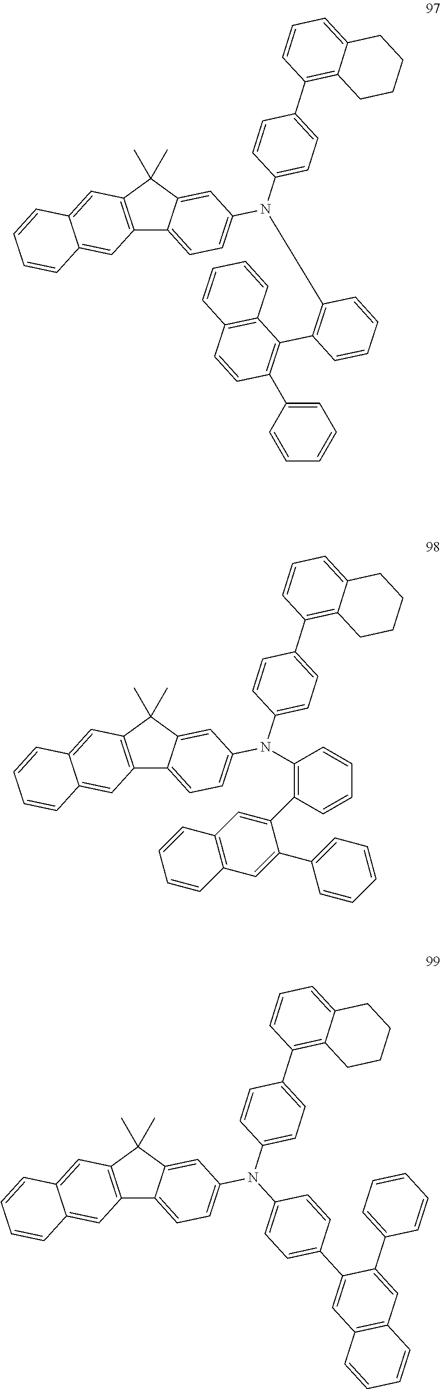 [SMILES: CC1(C)c2cc(N(c3ccc(-c4cccc5c4CCCC5)cc3)c3ccccc3-c3c(-c4ccccc4)ccc4ccccc34)ccc2-c2cc3ccccc3cc21 ; CC1(C)c2cc(N(c3ccc(-c4cccc5c4CCCC5)cc3)c3ccccc3-c3cc4ccccc4cc3-c3ccccc3)ccc2-c2cc3ccccc3cc21 ; CC1(C)c2cc(N(c3ccc(-c4cc5ccccc5cc4-c4ccccc4)cc3)c3ccc(-c4cccc5c4CCCC5)cc3)ccc2-c2cc3ccccc3cc21]

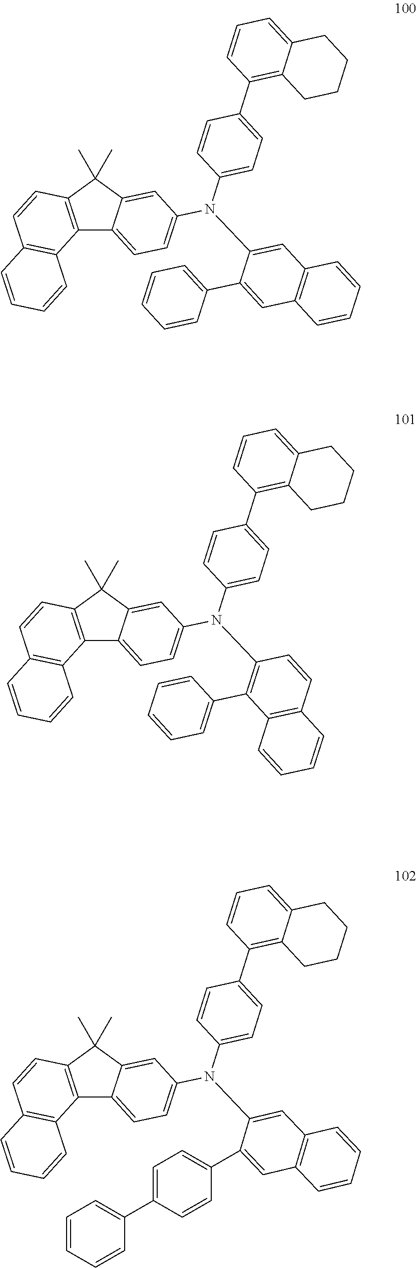 [SMILES: CC1(C)c2cc(N(c3ccc(-c4cccc5c4CCCC5)cc3)c3cc4ccccc4cc3-c3ccccc3)ccc2-c2c1ccc1ccccc21 ; CC1(C)c2cc(N(c3ccc(-c4cccc5c4CCCC5)cc3)c3ccc4ccccc4c3-c3ccccc3)ccc2-c2c1ccc1ccccc21 ; CC1(C)c2cc(N(c3ccc(-c4cccc5c4CCCC5)cc3)c3cc4ccccc4cc3-c3ccc(-c4ccccc4)cc3)ccc2-c2c1ccc1ccccc21]

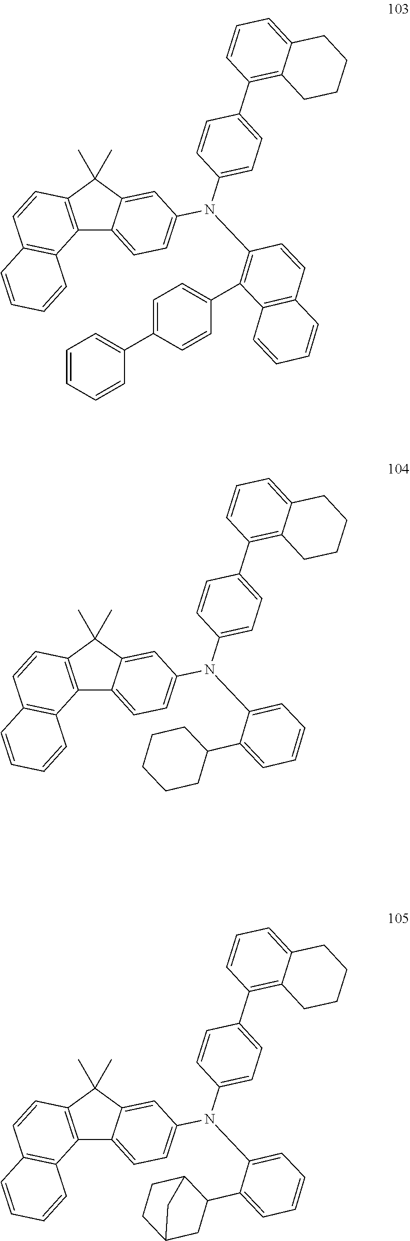 [SMILES: CC1(C)c2cc(N(c3ccc(-c4cccc5c4CCCC5)cc3)c3ccc4ccccc4c3-c3ccc(-c4ccccc4)cc3)ccc2-c2c1ccc1ccccc21 ; CC1(C)c2cc(N(c3ccc(-c4cccc5c4CCCC5)cc3)c3ccccc3C3CCCCC3)ccc2-c2c1ccc1ccccc21 ; CC1(C)c2cc(N(c3ccc(-c4cccc5c4CCCC5)cc3)c3ccccc3C3CC4CCC3C4)ccc2-c2c1ccc1ccccc21]

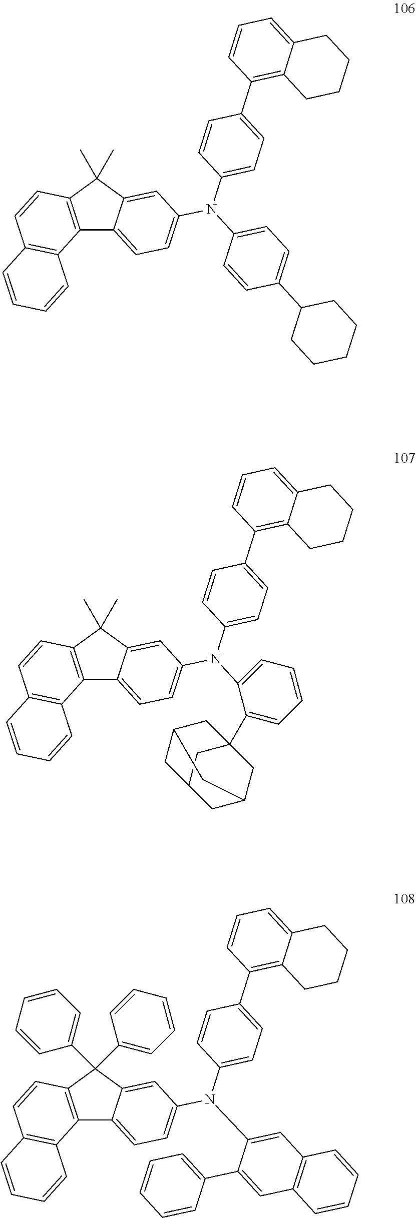 [SMILES: CC1(C)c2cc(N(c3ccc(-c4cccc5c4CCCC5)cc3)c3ccc(C4CCCCC4)cc3)ccc2-c2c1ccc1ccccc21 ; CC1(C)c2cc(N(c3ccc(-c4cccc5c4CCCC5)cc3)c3ccccc3C34CC5CC(CC(C5)C3)C4)ccc2-c2c1ccc1ccccc21 ; c1ccc(-c2cc3ccccc3cc2N(c2ccc(-c3cccc4c3CCCC4)cc2)c2ccc3c(c2)C(c2ccccc2)(c2ccccc2)c2ccc4ccccc4c2-3)cc1]

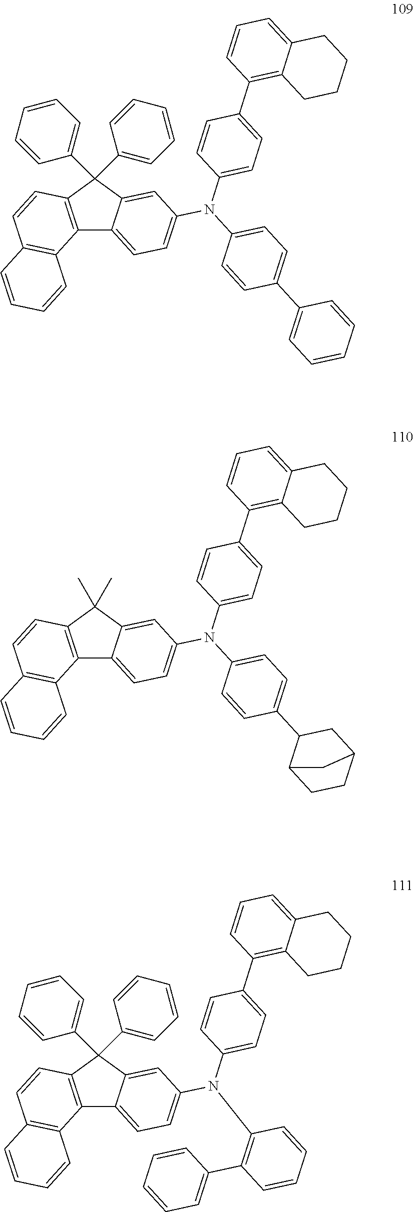 [SMILES: c1ccc(-c2ccc(N(c3ccc(-c4cccc5c4CCCC5)cc3)c3ccc4c(c3)C(c3ccccc3)(c3ccccc3)c3ccc5ccccc5c3-4)cc2)cc1 ; CC1(C)c2cc(N(c3ccc(-c4cccc5c4CCCC5)cc3)c3ccc(C4CC5CCC4C5)cc3)ccc2-c2c1ccc1ccccc21 ; c1ccc(-c2ccccc2N(c2ccc(-c3cccc4c3CCCC4)cc2)c2ccc3c(c2)C(c2ccccc2)(c2ccccc2)c2ccc4ccccc4c2-3)cc1]

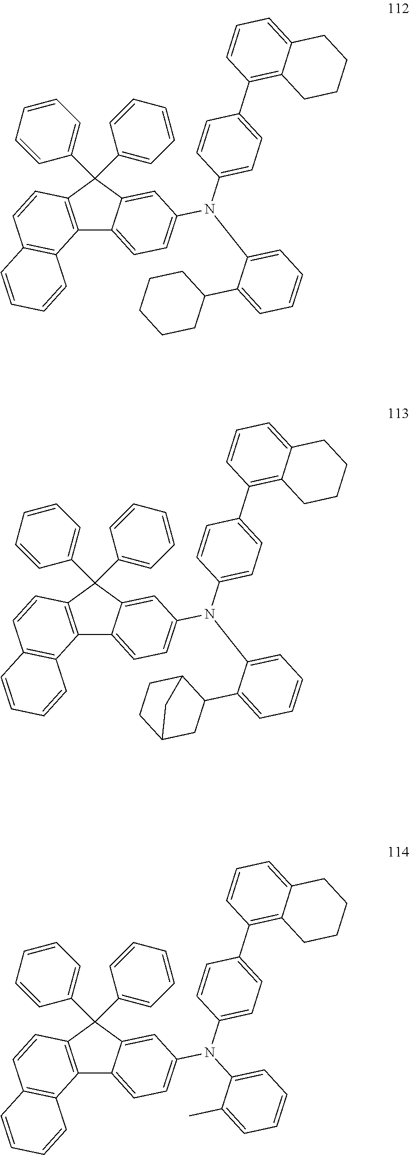 [SMILES: c1ccc(C2(c3ccccc3)c3cc(N(c4ccc(-c5cccc6c5CCCC6)cc4)c4ccccc4C4CCCCC4)ccc3-c3c2ccc2ccccc32)cc1 ; c1ccc(C2(c3ccccc3)c3cc(N(c4ccc(-c5cccc6c5CCCC6)cc4)c4ccccc4C4CC5CCC4C5)ccc3-c3c2ccc2ccccc32)cc1 ; Cc1ccccc1N(c1ccc(-c2cccc3c2CCCC3)cc1)c1ccc2c(c1)C(c1ccccc1)(c1ccccc1)c1ccc3ccccc3c1-2]

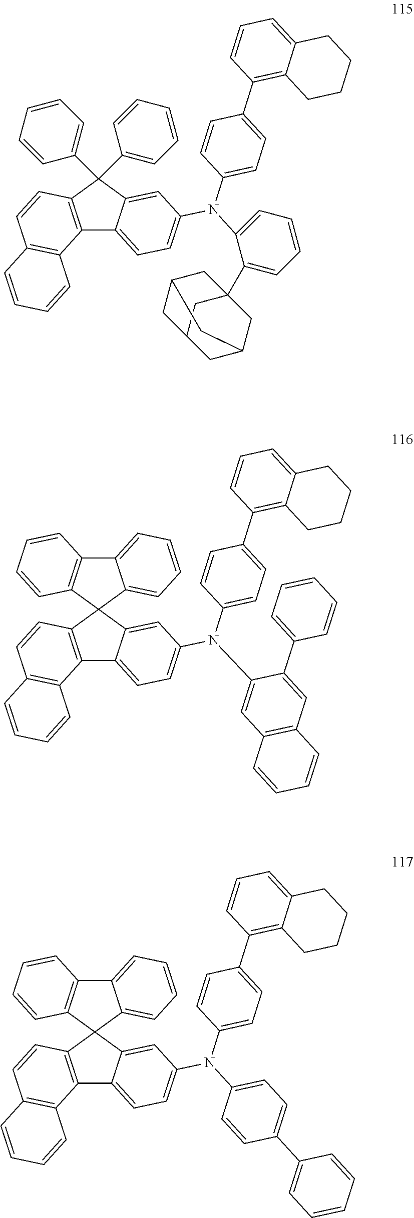 [SMILES: c1ccc(C2(c3ccccc3)c3cc(N(c4ccc(-c5cccc6c5CCCC6)cc4)c4ccccc4C45CC6CC(CC(C6)C4)C5)ccc3-c3c2ccc2ccccc32)cc1 ; c1ccc(-c2cc3ccccc3cc2N(c2ccc(-c3cccc4c3CCCC4)cc2)c2ccc3c(c2)C2(c4ccccc4-c4ccccc42)c2ccc4ccccc4c2-3)cc1 ; c1ccc(-c2ccc(N(c3ccc(-c4cccc5c4CCCC5)cc3)c3ccc4c(c3)C3(c5ccccc5-c5ccccc53)c3ccc5ccccc5c3-4)cc2)cc1]

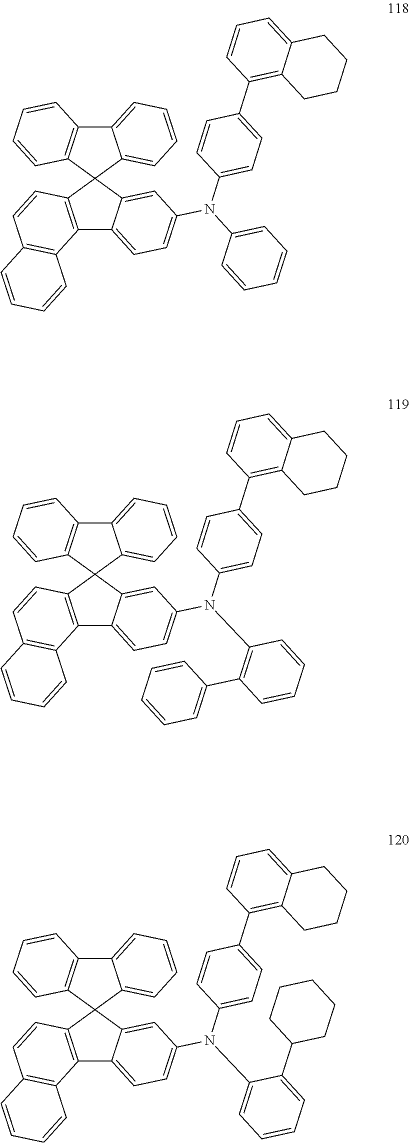 [SMILES: c1ccc(N(c2ccc(-c3cccc4c3CCCC4)cc2)c2ccc3c(c2)C2(c4ccccc4-c4ccccc42)c2ccc4ccccc4c2-3)cc1 ; c1ccc(-c2ccccc2N(c2ccc(-c3cccc4c3CCCC4)cc2)c2ccc3c(c2)C2(c4ccccc4-c4ccccc42)c2ccc4ccccc4c2-3)cc1 ; c1ccc2c(c1)-c1ccccc1C21c2cc(N(c3ccc(-c4cccc5c4CCCC5)cc3)c3ccccc3C3CCCCC3)ccc2-c2c1ccc1ccccc21]

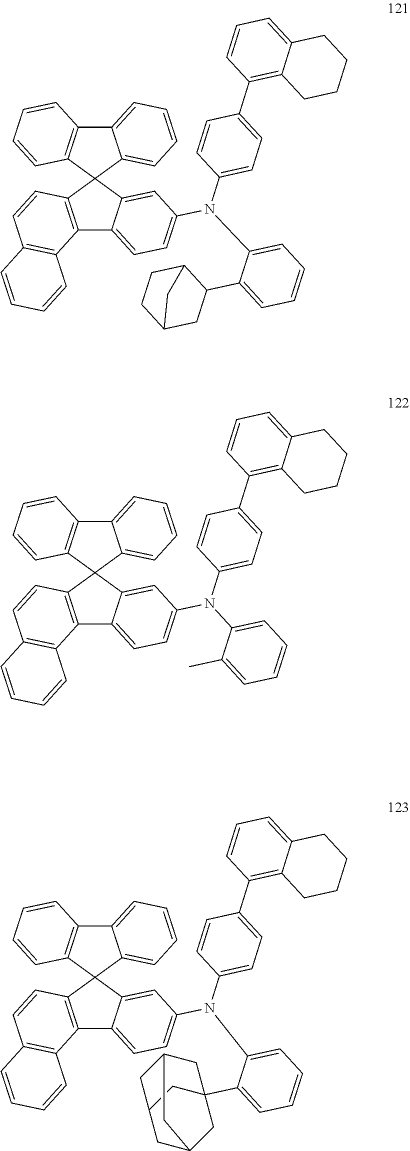 [SMILES: c1ccc2c(c1)-c1ccccc1C21c2cc(N(c3ccc(-c4cccc5c4CCCC5)cc3)c3ccccc3C3CC4CCC3C4)ccc2-c2c1ccc1ccccc21 ; Cc1ccccc1N(c1ccc(-c2cccc3c2CCCC3)cc1)c1ccc2c(c1)C1(c3ccccc3-c3ccccc31)c1ccc3ccccc3c1-2 ; c1ccc2c(c1)-c1ccccc1C21c2cc(N(c3ccc(-c4cccc5c4CCCC5)cc3)c3ccccc3C34CC5CC(CC(C5)C3)C4)ccc2-c2c1ccc1ccccc21]

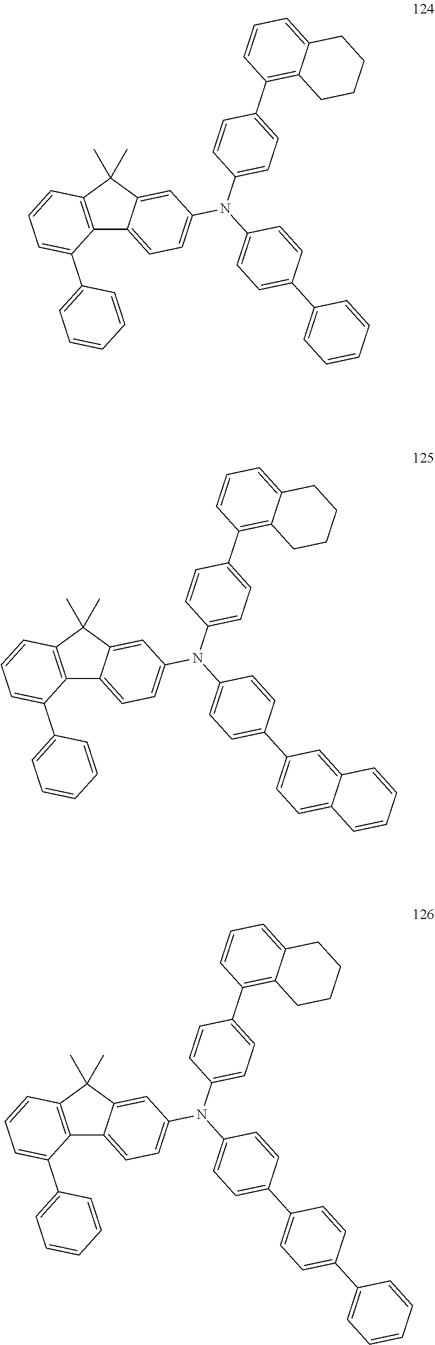 [SMILES: CC1(C)c2cc(N(c3ccc(-c4ccccc4)cc3)c3ccc(-c4cccc5c4CCCC5)cc3)ccc2-c2c(-c3ccccc3)cccc21 ; CC1(C)c2cc(N(c3ccc(-c4ccc5ccccc5c4)cc3)c3ccc(-c4cccc5c4CCCC5)cc3)ccc2-c2c(-c3ccccc3)cccc21 ; CC1(C)c2cc(N(c3ccc(-c4ccc(-c5ccccc5)cc4)cc3)c3ccc(-c4cccc5c4CCCC5)cc3)ccc2-c2c(-c3ccccc3)cccc21]

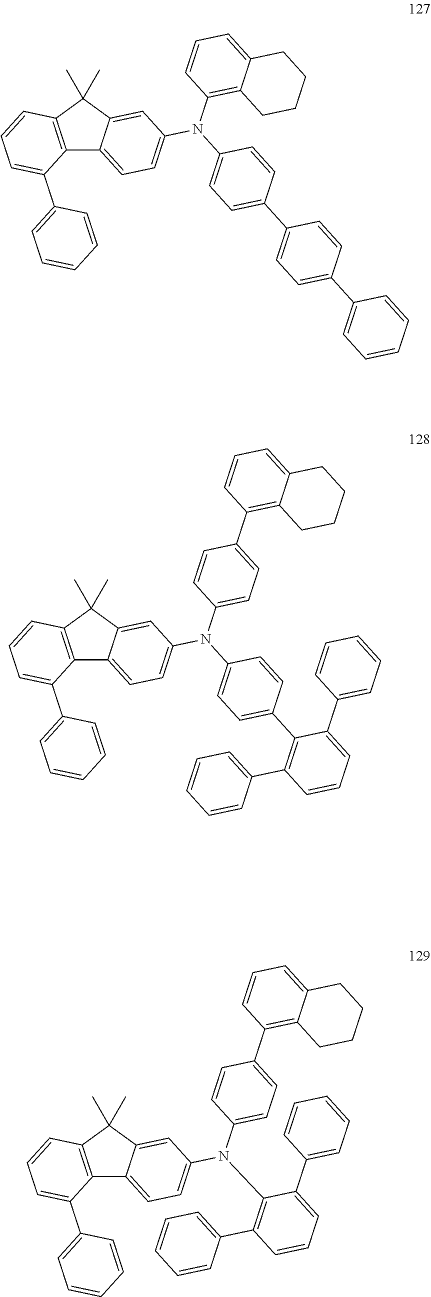 [SMILES: CC1(C)c2cc(N(c3ccc(-c4ccc(-c5ccccc5)cc4)cc3)c3cccc4c3CCCC4)ccc2-c2c(-c3ccccc3)cccc21 ; CC1(C)c2cc(N(c3ccc(-c4cccc5c4CCCC5)cc3)c3ccc(-c4c(-c5ccccc5)cccc4-c4ccccc4)cc3)ccc2-c2c(-c3ccccc3)cccc21 ; CC1(C)c2cc(N(c3ccc(-c4cccc5c4CCCC5)cc3)c3c(-c4ccccc4)cccc3-c3ccccc3)ccc2-c2c(-c3ccccc3)cccc21]

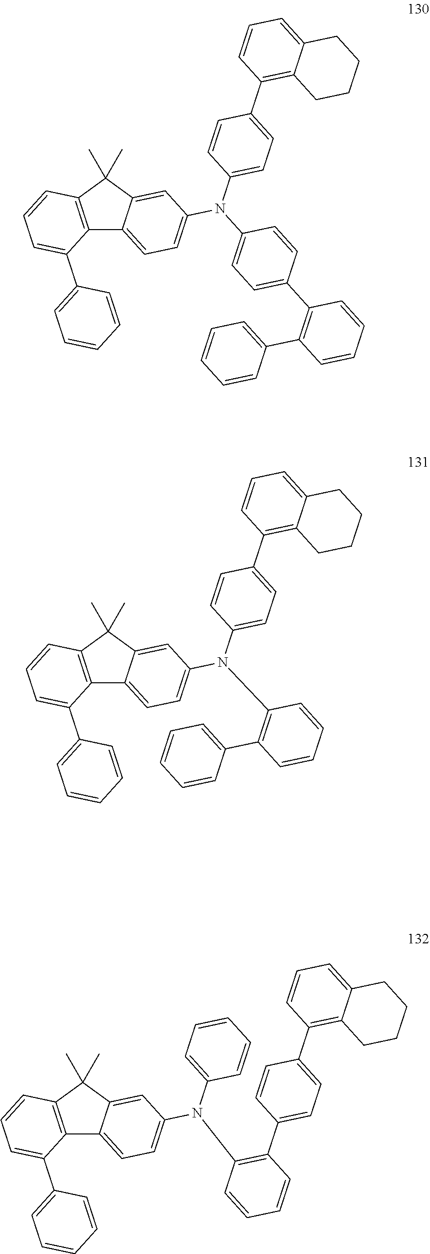 [SMILES: CC1(C)c2cc(N(c3ccc(-c4ccccc4-c4ccccc4)cc3)c3ccc(-c4cccc5c4CCCC5)cc3)ccc2-c2c(-c3ccccc3)cccc21 ; CC1(C)c2cc(N(c3ccc(-c4cccc5c4CCCC5)cc3)c3ccccc3-c3ccccc3)ccc2-c2c(-c3ccccc3)cccc21 ; CC1(C)c2cc(N(c3ccccc3)c3ccccc3-c3ccc(-c4cccc5c4CCCC5)cc3)ccc2-c2c(-c3ccccc3)cccc21]

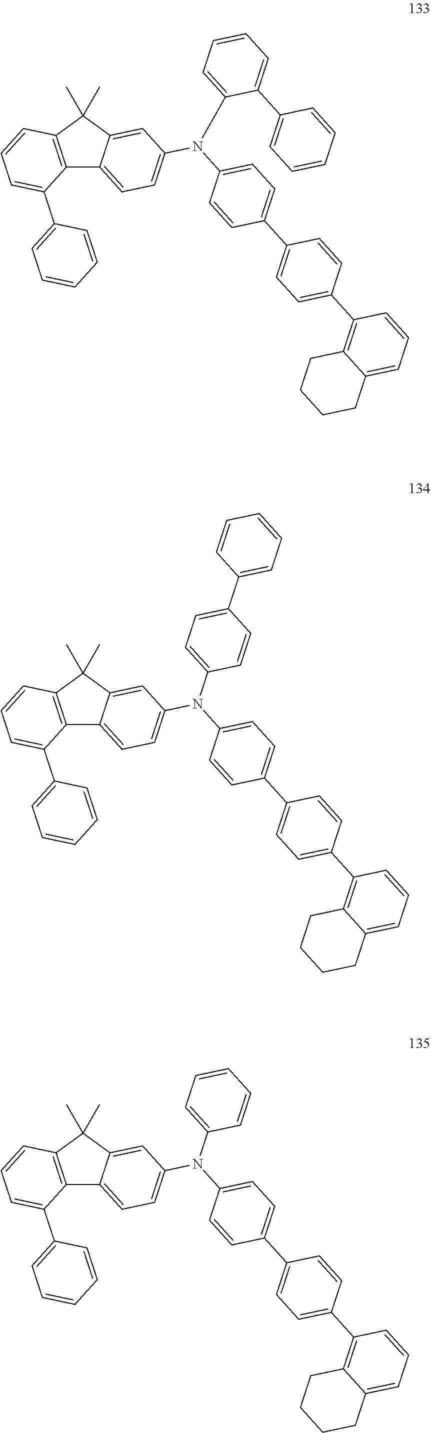 [SMILES: CC1(C)c2cc(N(c3ccc(-c4ccc(-c5cccc6c5CCCC6)cc4)cc3)c3ccccc3-c3ccccc3)ccc2-c2c(-c3ccccc3)cccc21 ; CC1(C)c2cc(N(c3ccc(-c4ccccc4)cc3)c3ccc(-c4ccc(-c5cccc6c5CCCC6)cc4)cc3)ccc2-c2c(-c3ccccc3)cccc21 ; CC1(C)c2cc(N(c3ccccc3)c3ccc(-c4ccc(-c5cccc6c5CCCC6)cc4)cc3)ccc2-c2c(-c3ccccc3)cccc21]

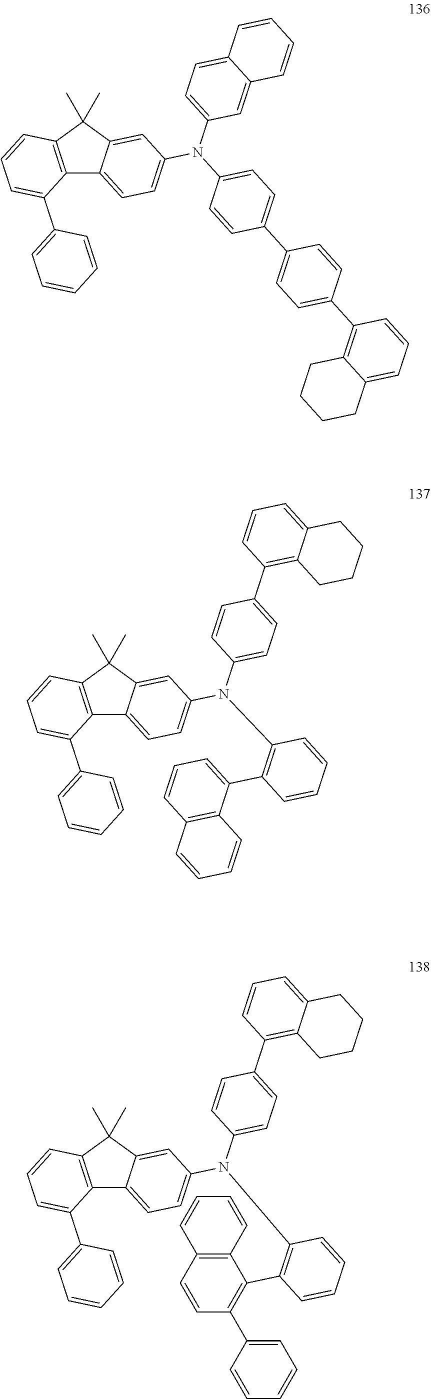 [SMILES: CC1(C)c2cc(N(c3ccc(-c4ccc(-c5cccc6c5CCCC6)cc4)cc3)c3ccc4ccccc4c3)ccc2-c2c(-c3ccccc3)cccc21 ; CC1(C)c2cc(N(c3ccc(-c4cccc5c4CCCC5)cc3)c3ccccc3-c3cccc4ccccc34)ccc2-c2c(-c3ccccc3)cccc21 ; CC1(C)c2cc(N(c3ccc(-c4cccc5c4CCCC5)cc3)c3ccccc3-c3c(-c4ccccc4)ccc4ccccc34)ccc2-c2c(-c3ccccc3)cccc21]

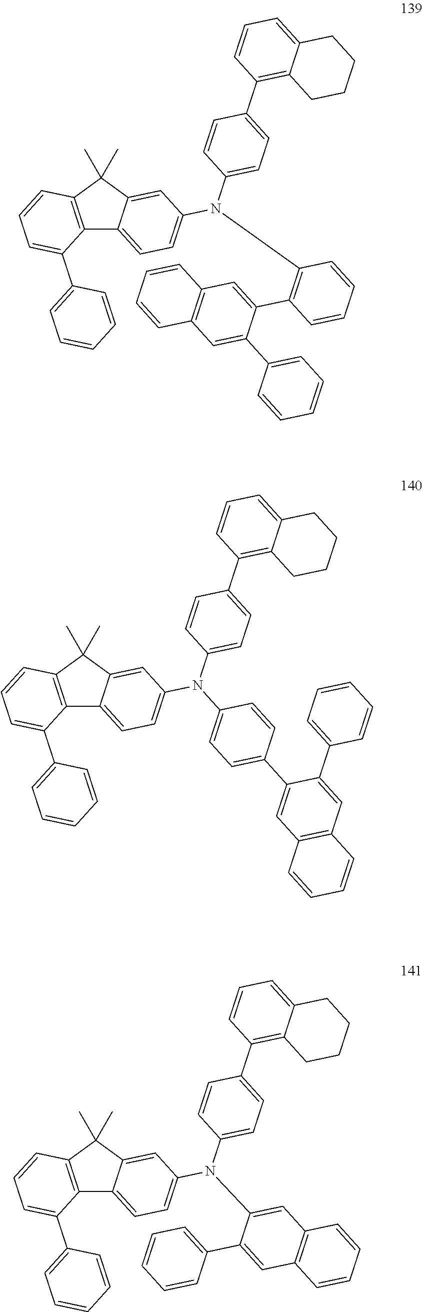 [SMILES: CC1(C)c2cc(N(c3ccc(-c4cccc5c4CCCC5)cc3)c3ccccc3-c3cc4ccccc4cc3-c3ccccc3)ccc2-c2c(-c3ccccc3)cccc21 ; CC1(C)c2cc(N(c3ccc(-c4cc5ccccc5cc4-c4ccccc4)cc3)c3ccc(-c4cccc5c4CCCC5)cc3)ccc2-c2c(-c3ccccc3)cccc21 ; CC1(C)c2cc(N(c3ccc(-c4cccc5c4CCCC5)cc3)c3cc4ccccc4cc3-c3ccccc3)ccc2-c2c(-c3ccccc3)cccc21]

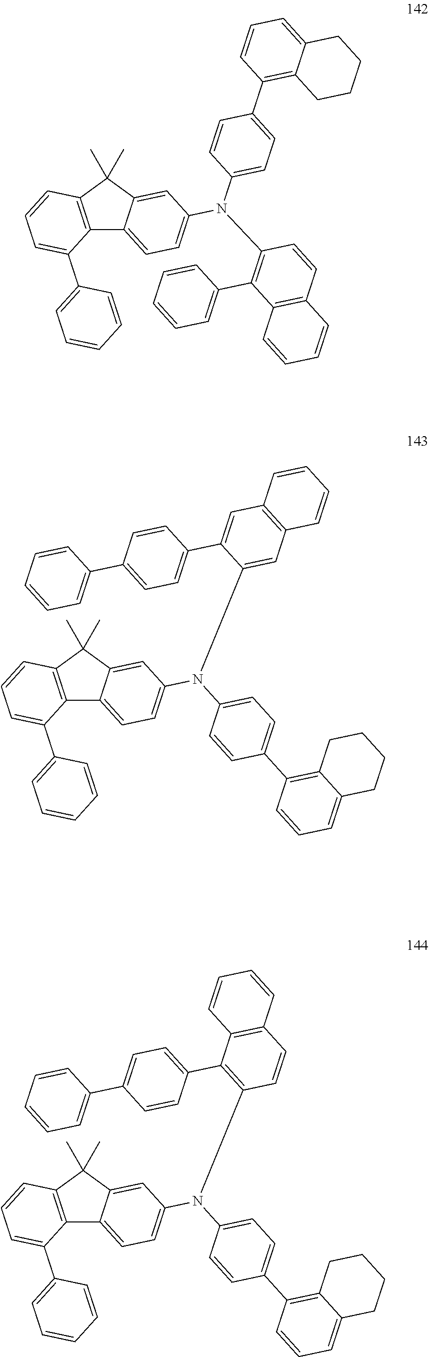 [SMILES: CC1(C)c2cc(N(c3ccc(-c4cccc5c4CCCC5)cc3)c3ccc4ccccc4c3-c3ccccc3)ccc2-c2c(-c3ccccc3)cccc21 ; CC1(C)c2cc(N(c3ccc(-c4cccc5c4CCCC5)cc3)c3cc4ccccc4cc3-c3ccc(-c4ccccc4)cc3)ccc2-c2c(-c3ccccc3)cccc21 ; CC1(C)c2cc(N(c3ccc(-c4cccc5c4CCCC5)cc3)c3ccc4ccccc4c3-c3ccc(-c4ccccc4)cc3)ccc2-c2c(-c3ccccc3)cccc21]

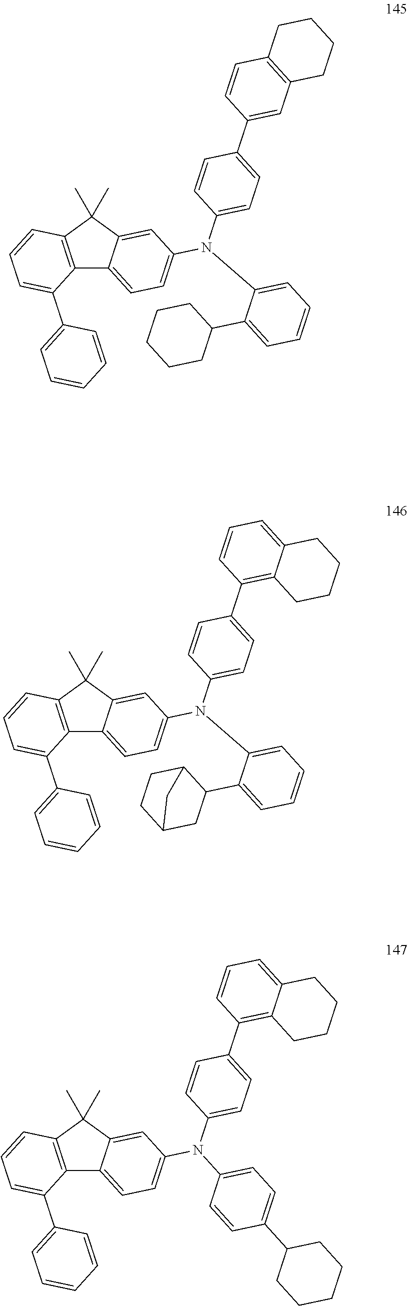 [SMILES: CC1(C)c2cc(N(c3ccc(-c4ccc5c(c4)CCCC5)cc3)c3ccccc3C3CCCCC3)ccc2-c2c(-c3ccccc3)cccc21 ; CC1(C)c2cc(N(c3ccc(-c4cccc5c4CCCC5)cc3)c3ccccc3C3CC4CCC3C4)ccc2-c2c(-c3ccccc3)cccc21 ; CC1(C)c2cc(N(c3ccc(-c4cccc5c4CCCC5)cc3)c3ccc(C4CCCCC4)cc3)ccc2-c2c(-c3ccccc3)cccc21]

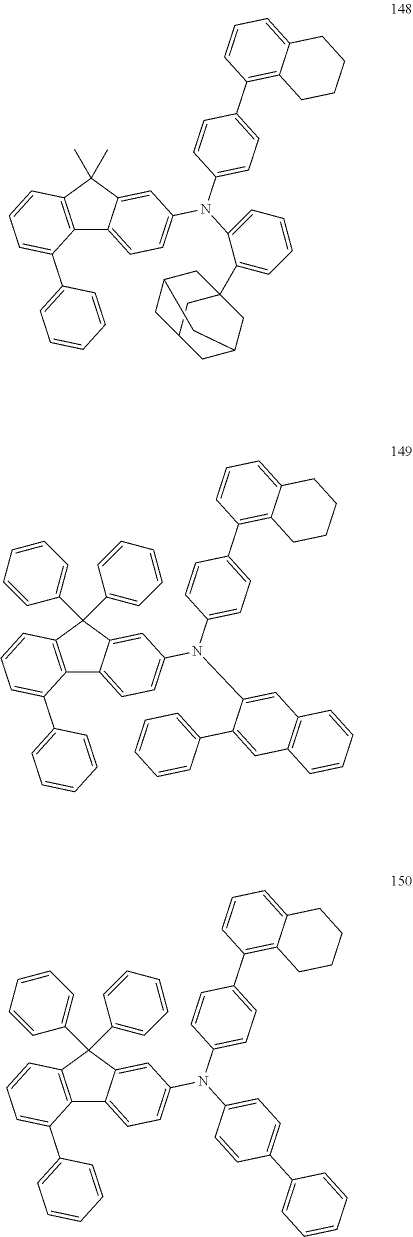 [SMILES: CC1(C)c2cc(N(c3ccc(-c4cccc5c4CCCC5)cc3)c3ccccc3C34CC5CC(CC(C5)C3)C4)ccc2-c2c(-c3ccccc3)cccc21 ; c1ccc(-c2cc3ccccc3cc2N(c2ccc(-c3cccc4c3CCCC4)cc2)c2ccc3c(c2)C(c2ccccc2)(c2ccccc2)c2cccc(-c4ccccc4)c2-3)cc1 ; c1ccc(-c2ccc(N(c3ccc(-c4cccc5c4CCCC5)cc3)c3ccc4c(c3)C(c3ccccc3)(c3ccccc3)c3cccc(-c5ccccc5)c3-4)cc2)cc1]

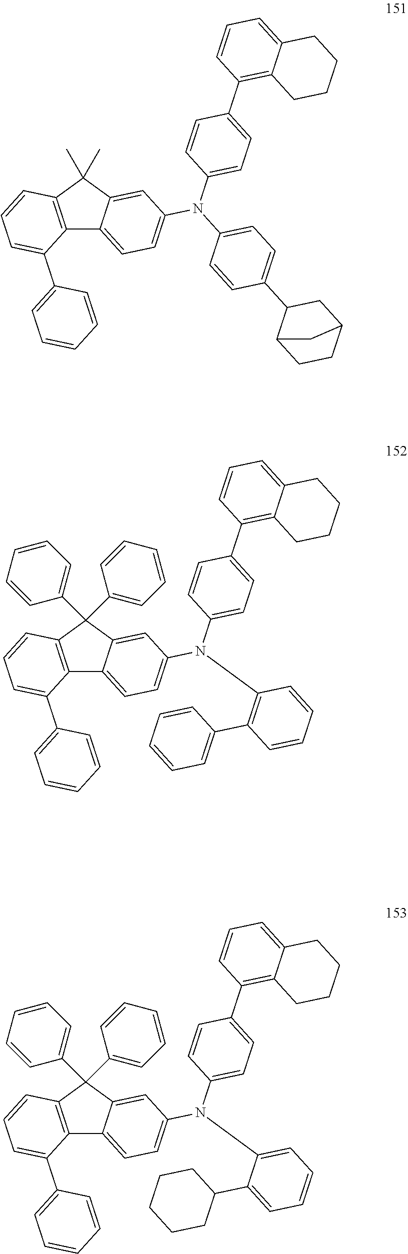 [SMILES: CC1(C)c2cc(N(c3ccc(-c4cccc5c4CCCC5)cc3)c3ccc(C4CC5CCC4C5)cc3)ccc2-c2c(-c3ccccc3)cccc21 ; c1ccc(-c2ccccc2N(c2ccc(-c3cccc4c3CCCC4)cc2)c2ccc3c(c2)C(c2ccccc2)(c2ccccc2)c2cccc(-c4ccccc4)c2-3)cc1 ; c1ccc(-c2cccc3c2-c2ccc(N(c4ccc(-c5cccc6c5CCCC6)cc4)c4ccccc4C4CCCCC4)cc2C3(c2ccccc2)c2ccccc2)cc1]

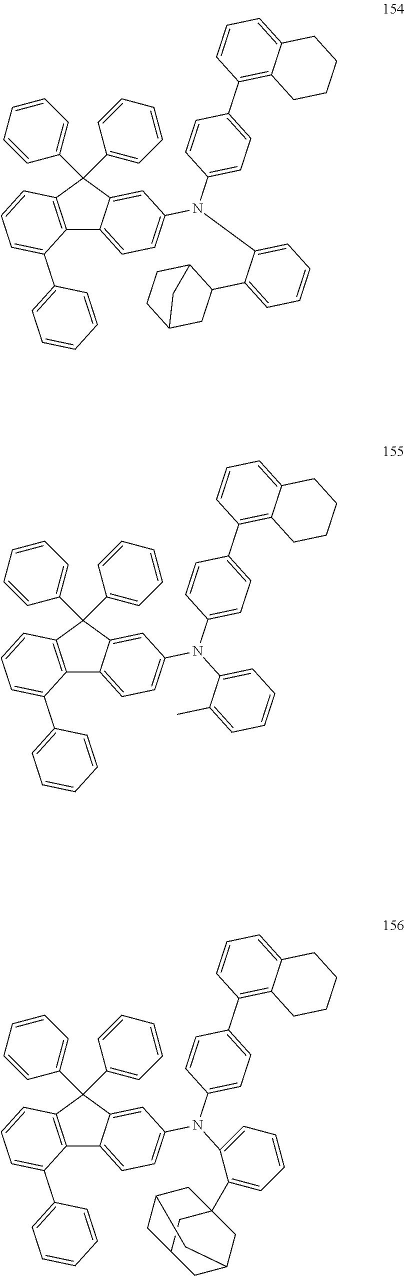 [SMILES: c1ccc(-c2cccc3c2-c2ccc(N(c4ccc(-c5cccc6c5CCCC6)cc4)c4ccccc4C4CC5CCC4C5)cc2C3(c2ccccc2)c2ccccc2)cc1 ; Cc1ccccc1N(c1ccc(-c2cccc3c2CCCC3)cc1)c1ccc2c(c1)C(c1ccccc1)(c1ccccc1)c1cccc(-c3ccccc3)c1-2 ; c1ccc(-c2cccc3c2-c2ccc(N(c4ccc(-c5cccc6c5CCCC6)cc4)c4ccccc4C45CC6CC(CC(C6)C4)C5)cc2C3(c2ccccc2)c2ccccc2)cc1]

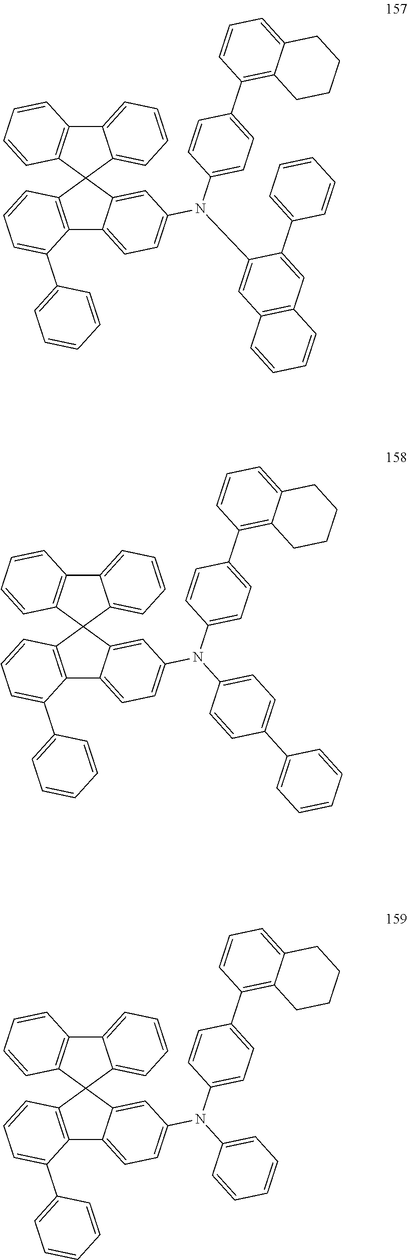 [SMILES: c1ccc(-c2cc3ccccc3cc2N(c2ccc(-c3cccc4c3CCCC4)cc2)c2ccc3c(c2)C2(c4ccccc4-c4ccccc42)c2cccc(-c4ccccc4)c2-3)cc1 ; c1ccc(-c2ccc(N(c3ccc(-c4cccc5c4CCCC5)cc3)c3ccc4c(c3)C3(c5ccccc5-c5ccccc53)c3cccc(-c5ccccc5)c3-4)cc2)cc1 ; c1ccc(-c2cccc3c2-c2ccc(N(c4ccccc4)c4ccc(-c5cccc6c5CCCC6)cc4)cc2C32c3ccccc3-c3ccccc32)cc1]

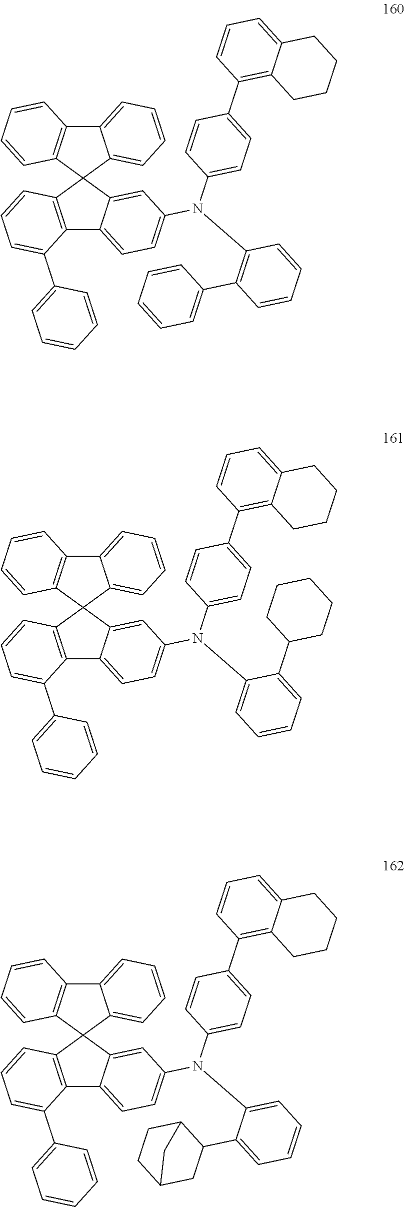 [SMILES: c1ccc(-c2ccccc2N(c2ccc(-c3cccc4c3CCCC4)cc2)c2ccc3c(c2)C2(c4ccccc4-c4ccccc42)c2cccc(-c4ccccc4)c2-3)cc1 ; c1ccc(-c2cccc3c2-c2ccc(N(c4ccc(-c5cccc6c5CCCC6)cc4)c4ccccc4C4CCCCC4)cc2C32c3ccccc3-c3ccccc32)cc1 ; c1ccc(-c2cccc3c2-c2ccc(N(c4ccc(-c5cccc6c5CCCC6)cc4)c4ccccc4C4CC5CCC4C5)cc2C32c3ccccc3-c3ccccc32)cc1]

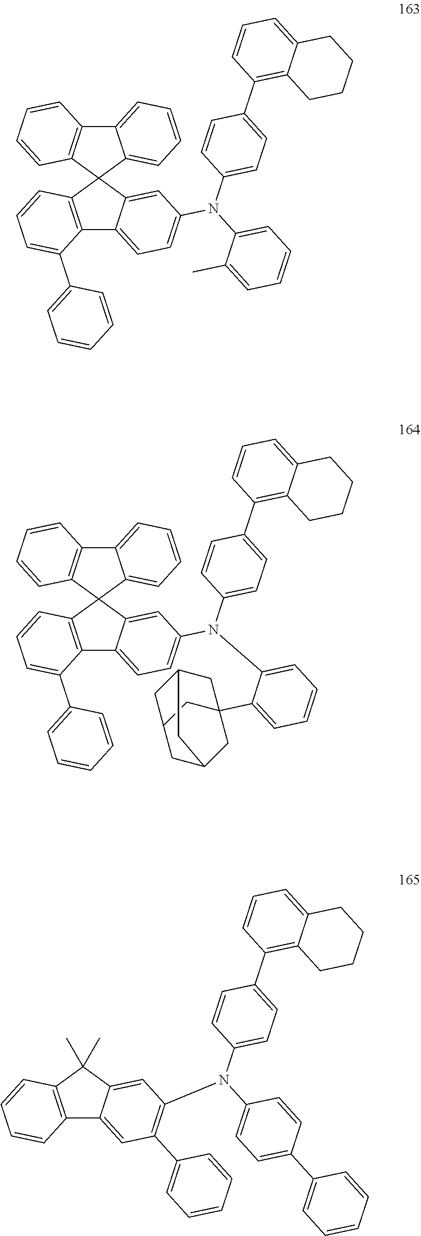 [SMILES: Cc1ccccc1N(c1ccc(-c2cccc3c2CCCC3)cc1)c1ccc2c(c1)C1(c3ccccc3-c3ccccc31)c1cccc(-c3ccccc3)c1-2 ; c1ccc(-c2cccc3c2-c2ccc(N(c4ccc(-c5cccc6c5CCCC6)cc4)c4ccccc4C45CC6CC(CC(C6)C4)C5)cc2C32c3ccccc3-c3ccccc32)cc1 ; CC1(C)c2ccccc2-c2cc(-c3ccccc3)c(N(c3ccc(-c4ccccc4)cc3)c3ccc(-c4cccc5c4CCCC5)cc3)cc21]

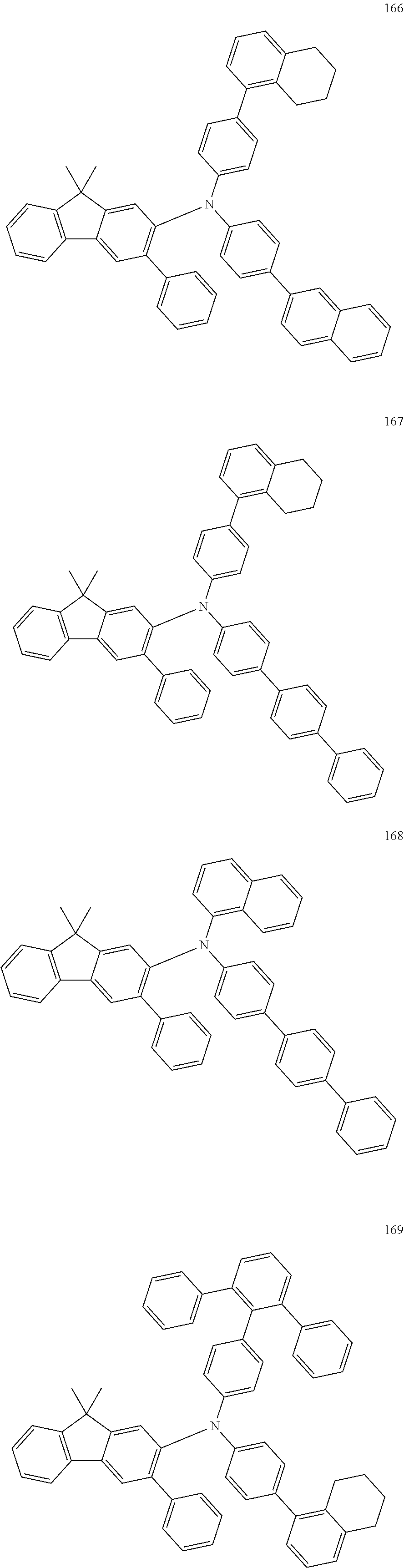 [SMILES: CC1(C)c2ccccc2-c2cc(-c3ccccc3)c(N(c3ccc(-c4ccc5ccccc5c4)cc3)c3ccc(-c4cccc5c4CCCC5)cc3)cc21 ; CC1(C)c2ccccc2-c2cc(-c3ccccc3)c(N(c3ccc(-c4ccc(-c5ccccc5)cc4)cc3)c3ccc(-c4cccc5c4CCCC5)cc3)cc21 ; CC1(C)c2ccccc2-c2cc(-c3ccccc3)c(N(c3ccc(-c4ccc(-c5ccccc5)cc4)cc3)c3cccc4ccccc34)cc21 ; CC1(C)c2ccccc2-c2cc(-c3ccccc3)c(N(c3ccc(-c4cccc5c4CCCC5)cc3)c3ccc(-c4c(-c5ccccc5)cccc4-c4ccccc4)cc3)cc21]

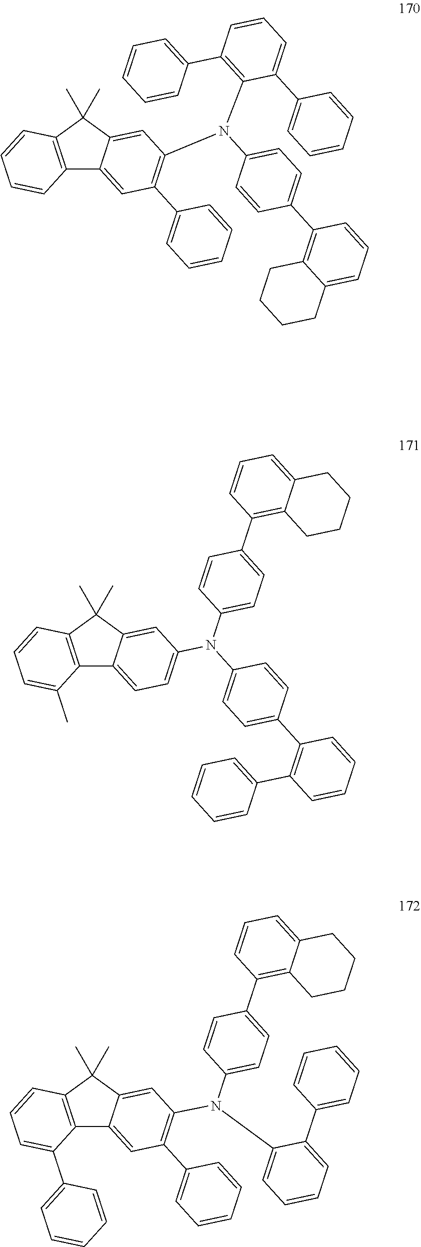 [SMILES: CC1(C)c2ccccc2-c2cc(-c3ccccc3)c(N(c3ccc(-c4cccc5c4CCCC5)cc3)c3c(-c4ccccc4)cccc3-c3ccccc3)cc21 ; Cc1cccc2c1-c1ccc(N(c3ccc(-c4ccccc4-c4ccccc4)cc3)c3ccc(-c4cccc5c4CCCC5)cc3)cc1C2(C)C ; CC1(C)c2cc(N(c3ccc(-c4cccc5c4CCCC5)cc3)c3ccccc3-c3ccccc3)c(-c3ccccc3)cc2-c2c(-c3ccccc3)cccc21]

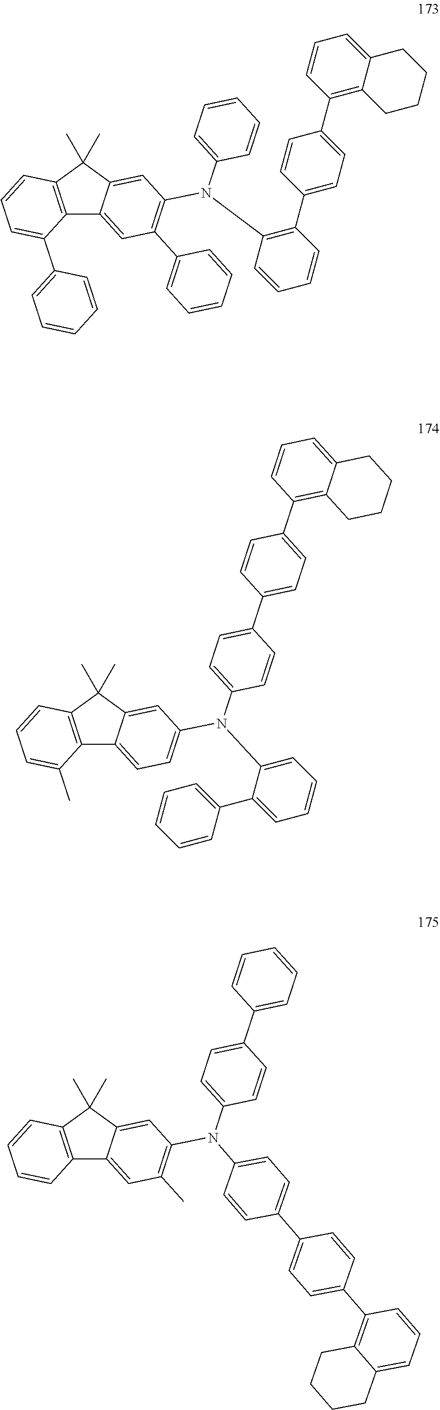 [SMILES: CC1(C)c2cc(N(c3ccccc3)c3ccccc3-c3ccc(-c4cccc5c4CCCC5)cc3)c(-c3ccccc3)cc2-c2c(-c3ccccc3)cccc21 ; Cc1cccc2c1-c1ccc(N(c3ccc(-c4ccc(-c5cccc6c5CCCC6)cc4)cc3)c3ccccc3-c3ccccc3)cc1C2(C)C ; Cc1cc2c(cc1N(c1ccc(-c3ccccc3)cc1)c1ccc(-c3ccc(-c4cccc5c4CCCC5)cc3)cc1)C(C)(C)c1ccccc1-2]

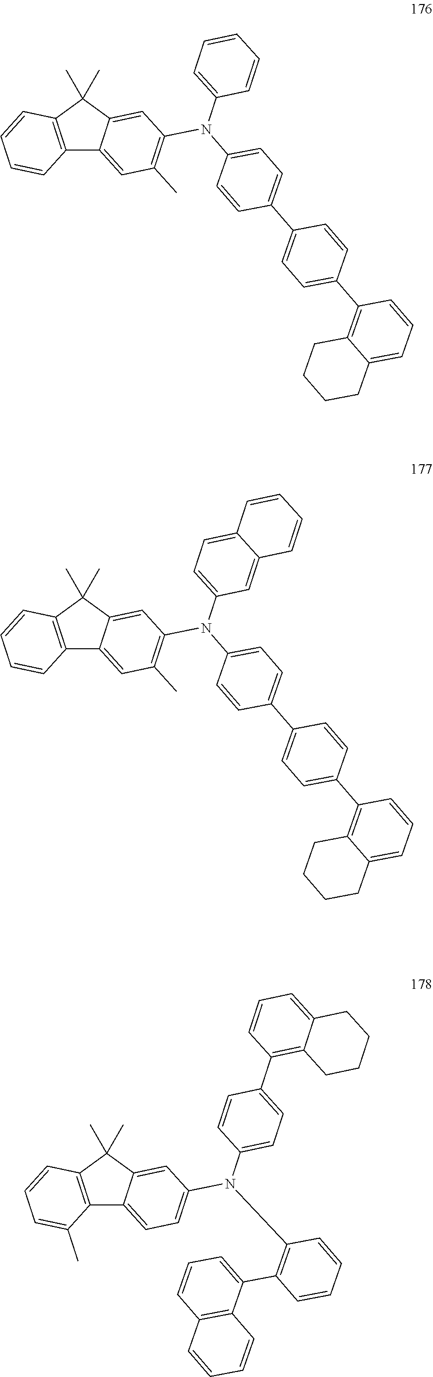 [SMILES: Cc1cc2c(cc1N(c1ccccc1)c1ccc(-c3ccc(-c4cccc5c4CCCC5)cc3)cc1)C(C)(C)c1ccccc1-2 ; Cc1cc2c(cc1N(c1ccc(-c3ccc(-c4cccc5c4CCCC5)cc3)cc1)c1ccc3ccccc3c1)C(C)(C)c1ccccc1-2 ; Cc1cccc2c1-c1ccc(N(c3ccc(-c4cccc5c4CCCC5)cc3)c3ccccc3-c3cccc4ccccc34)cc1C2(C)C]

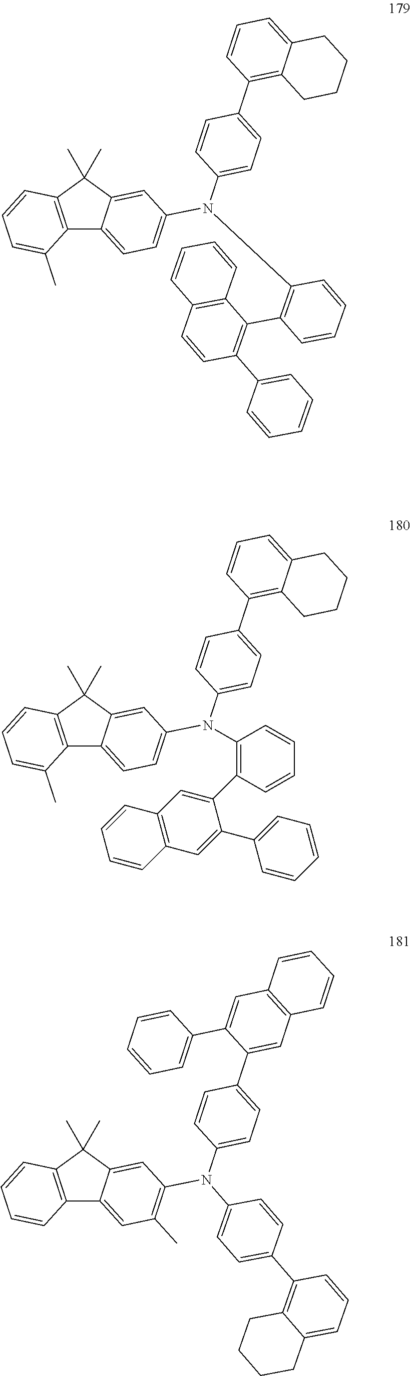 [SMILES: Cc1cccc2c1-c1ccc(N(c3ccc(-c4cccc5c4CCCC5)cc3)c3ccccc3-c3c(-c4ccccc4)ccc4ccccc34)cc1C2(C)C ; Cc1cccc2c1-c1ccc(N(c3ccc(-c4cccc5c4CCCC5)cc3)c3ccccc3-c3cc4ccccc4cc3-c3ccccc3)cc1C2(C)C ; Cc1cc2c(cc1N(c1ccc(-c3cc4ccccc4cc3-c3ccccc3)cc1)c1ccc(-c3cccc4c3CCCC4)cc1)C(C)(C)c1ccccc1-2]

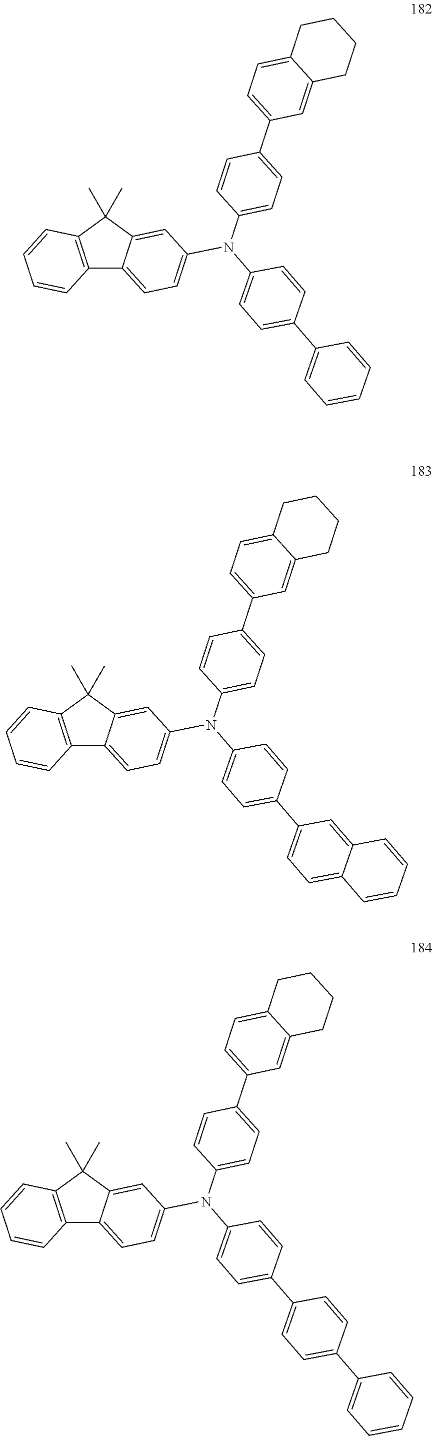 [SMILES: CC1(C)c2ccccc2-c2ccc(N(c3ccc(-c4ccccc4)cc3)c3ccc(-c4ccc5c(c4)CCCC5)cc3)cc21 ; CC1(C)c2ccccc2-c2ccc(N(c3ccc(-c4ccc5c(c4)CCCC5)cc3)c3ccc(-c4ccc5ccccc5c4)cc3)cc21 ; CC1(C)c2ccccc2-c2ccc(N(c3ccc(-c4ccc(-c5ccccc5)cc4)cc3)c3ccc(-c4ccc5c(c4)CCCC5)cc3)cc21]

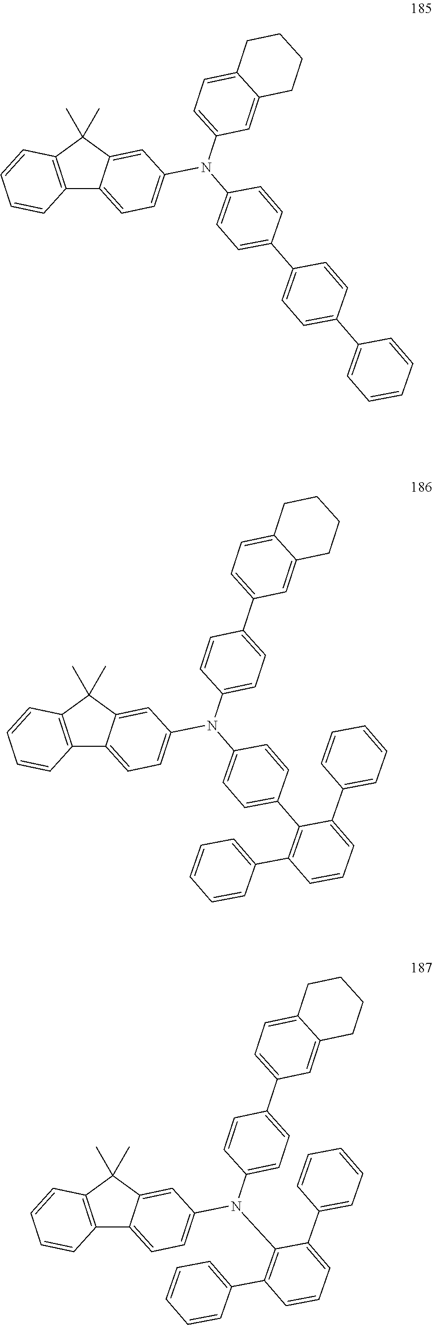 [SMILES: CC1(C)c2ccccc2-c2ccc(N(c3ccc(-c4ccc(-c5ccccc5)cc4)cc3)c3ccc4c(c3)CCCC4)cc21 ; CC1(C)c2ccccc2-c2ccc(N(c3ccc(-c4ccc5c(c4)CCCC5)cc3)c3ccc(-c4c(-c5ccccc5)cccc4-c4ccccc4)cc3)cc21 ; CC1(C)c2ccccc2-c2ccc(N(c3ccc(-c4ccc5c(c4)CCCC5)cc3)c3c(-c4ccccc4)cccc3-c3ccccc3)cc21]

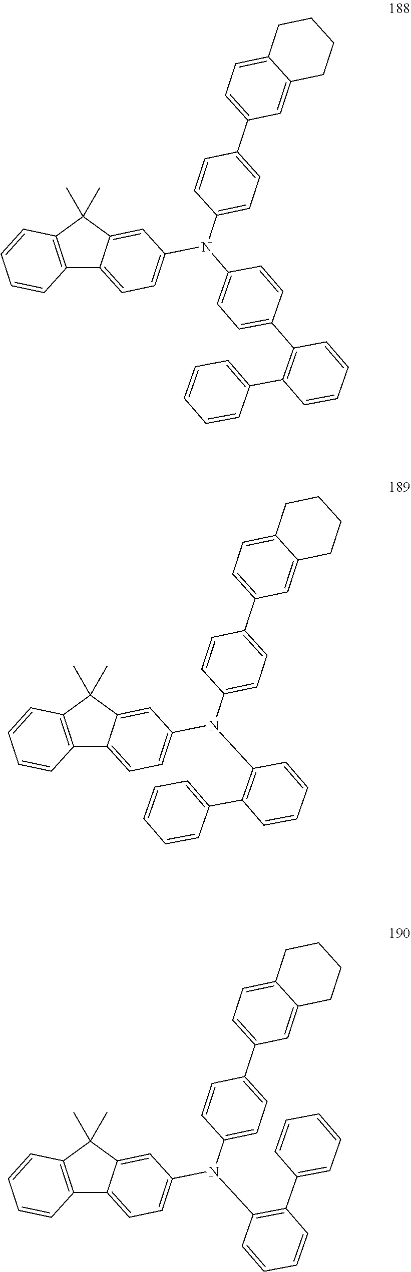 [SMILES: CC1(C)c2ccccc2-c2ccc(N(c3ccc(-c4ccc5c(c4)CCCC5)cc3)c3ccc(-c4ccccc4-c4ccccc4)cc3)cc21 ; CC1(C)c2ccccc2-c2ccc(N(c3ccc(-c4ccc5c(c4)CCCC5)cc3)c3ccccc3-c3ccccc3)cc21 ; CC1(C)c2ccccc2-c2ccc(N(c3ccc(-c4ccc5c(c4)CCCC5)cc3)c3ccccc3-c3ccccc3)cc21]

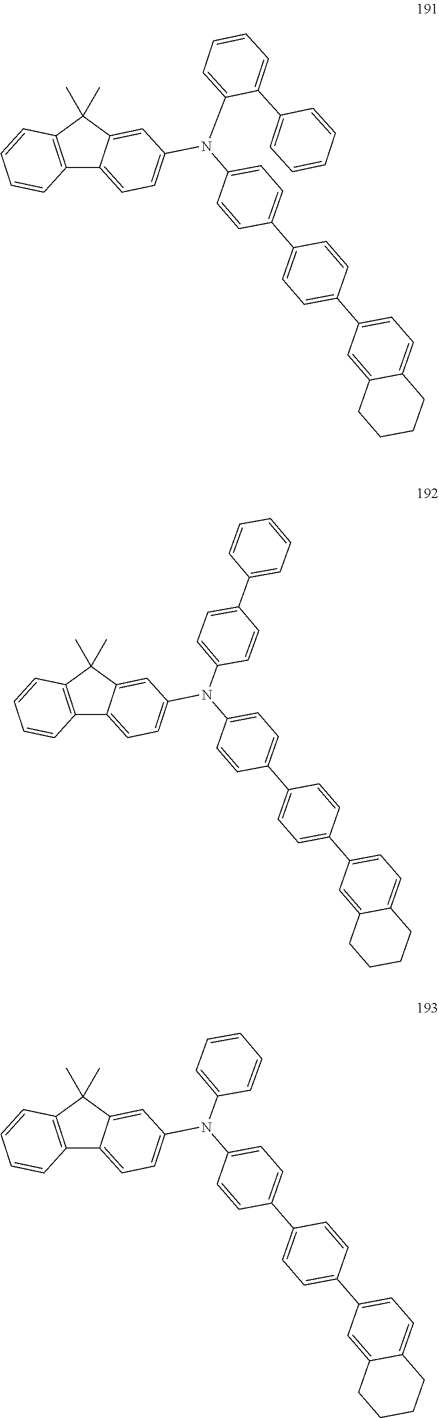 [SMILES: CC1(C)c2ccccc2-c2ccc(N(c3ccc(-c4ccc(-c5ccc6c(c5)CCCC6)cc4)cc3)c3ccccc3-c3ccccc3)cc21 ; CC1(C)c2ccccc2-c2ccc(N(c3ccc(-c4ccccc4)cc3)c3ccc(-c4ccc(-c5ccc6c(c5)CCCC6)cc4)cc3)cc21 ; CC1(C)c2ccccc2-c2ccc(N(c3ccccc3)c3ccc(-c4ccc(-c5ccc6c(c5)CCCC6)cc4)cc3)cc21]

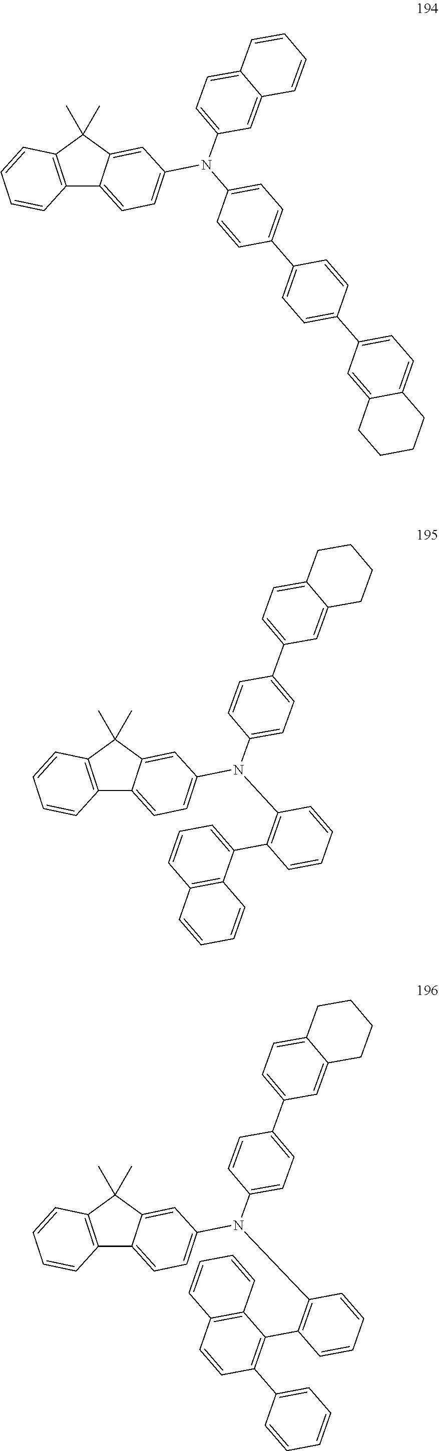 [SMILES: CC1(C)c2ccccc2-c2ccc(N(c3ccc(-c4ccc(-c5ccc6c(c5)CCCC6)cc4)cc3)c3ccc4ccccc4c3)cc21 ; CC1(C)c2ccccc2-c2ccc(N(c3ccc(-c4ccc5c(c4)CCCC5)cc3)c3ccccc3-c3cccc4ccccc34)cc21 ; CC1(C)c2ccccc2-c2ccc(N(c3ccc(-c4ccc5c(c4)CCCC5)cc3)c3ccccc3-c3c(-c4ccccc4)ccc4ccccc34)cc21]

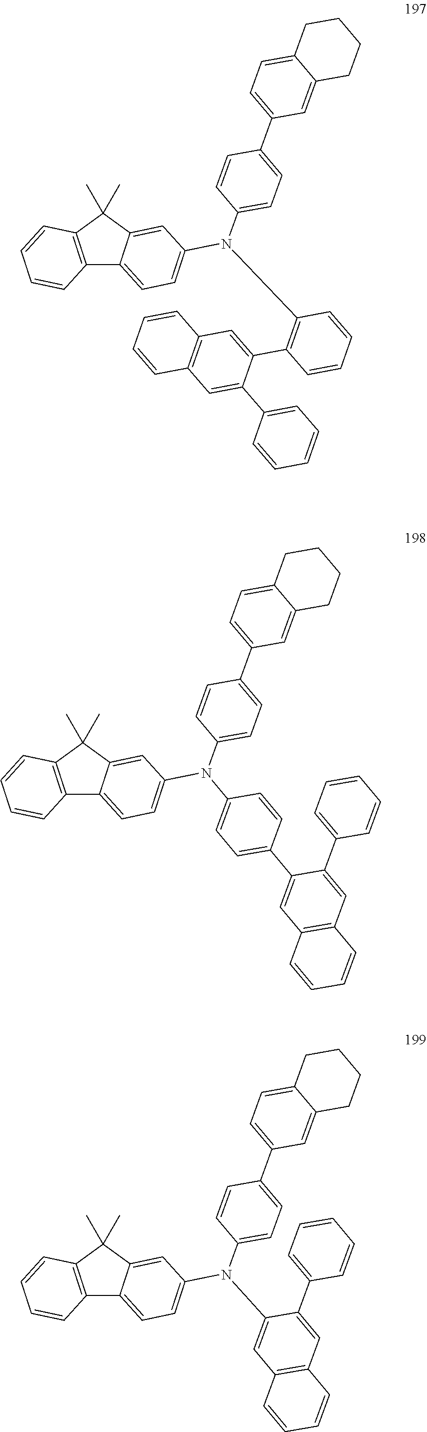 [SMILES: CC1(C)c2ccccc2-c2ccc(N(c3ccc(-c4ccc5c(c4)CCCC5)cc3)c3ccccc3-c3cc4ccccc4cc3-c3ccccc3)cc21 ; CC1(C)c2ccccc2-c2ccc(N(c3ccc(-c4ccc5c(c4)CCCC5)cc3)c3ccc(-c4cc5ccccc5cc4-c4ccccc4)cc3)cc21 ; CC1(C)c2ccccc2-c2ccc(N(c3ccc(-c4ccc5c(c4)CCCC5)cc3)c3cc4ccccc4cc3-c3ccccc3)cc21]

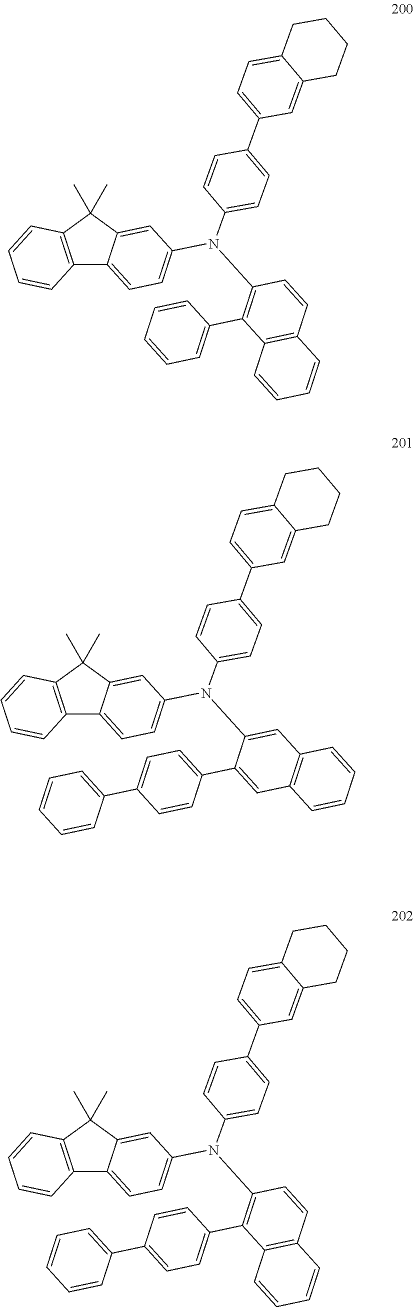 [SMILES: CC1(C)c2ccccc2-c2ccc(N(c3ccc(-c4ccc5c(c4)CCCC5)cc3)c3ccc4ccccc4c3-c3ccccc3)cc21 ; CC1(C)c2ccccc2-c2ccc(N(c3ccc(-c4ccc5c(c4)CCCC5)cc3)c3cc4ccccc4cc3-c3ccc(-c4ccccc4)cc3)cc21 ; CC1(C)c2ccccc2-c2ccc(N(c3ccc(-c4ccc5c(c4)CCCC5)cc3)c3ccc4ccccc4c3-c3ccc(-c4ccccc4)cc3)cc21]

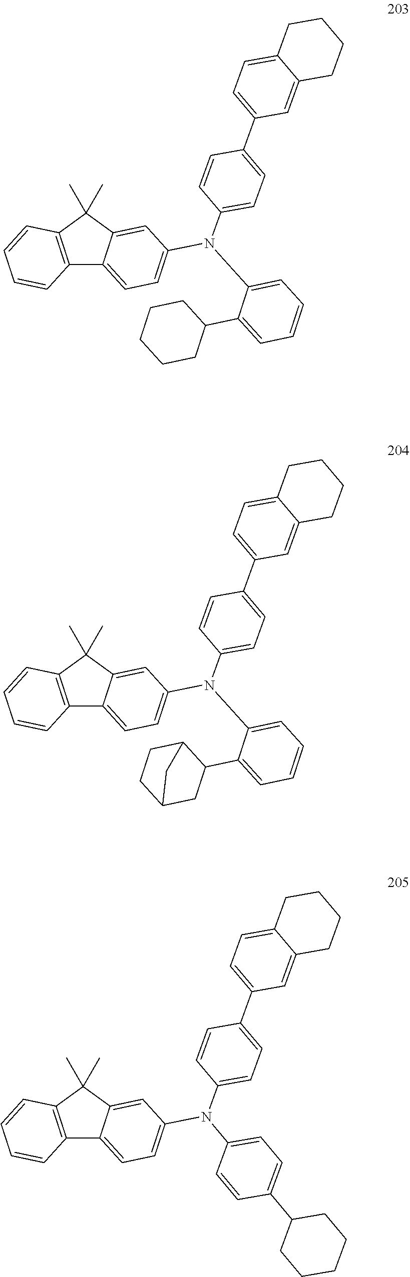 [SMILES: CC1(C)c2ccccc2-c2ccc(N(c3ccc(-c4ccc5c(c4)CCCC5)cc3)c3ccccc3C3CCCCC3)cc21 ; CC1(C)c2ccccc2-c2ccc(N(c3ccc(-c4ccc5c(c4)CCCC5)cc3)c3ccccc3C3CC4CCC3C4)cc21 ; CC1(C)c2ccccc2-c2ccc(N(c3ccc(-c4ccc5c(c4)CCCC5)cc3)c3ccc(C4CCCCC4)cc3)cc21]

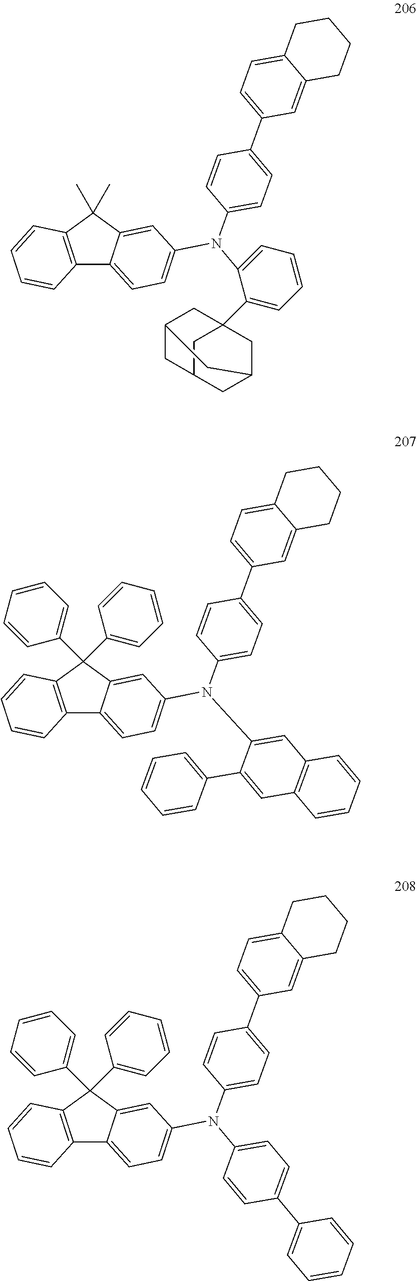 [SMILES: CC1(C)c2ccccc2-c2ccc(N(c3ccc(-c4ccc5c(c4)CCCC5)cc3)c3ccccc3C34CC5CC(CC(C5)C3)C4)cc21 ; c1ccc(-c2cc3ccccc3cc2N(c2ccc(-c3ccc4c(c3)CCCC4)cc2)c2ccc3c(c2)C(c2ccccc2)(c2ccccc2)c2ccccc2-3)cc1 ; c1ccc(-c2ccc(N(c3ccc(-c4ccc5c(c4)CCCC5)cc3)c3ccc4c(c3)C(c3ccccc3)(c3ccccc3)c3ccccc3-4)cc2)cc1]

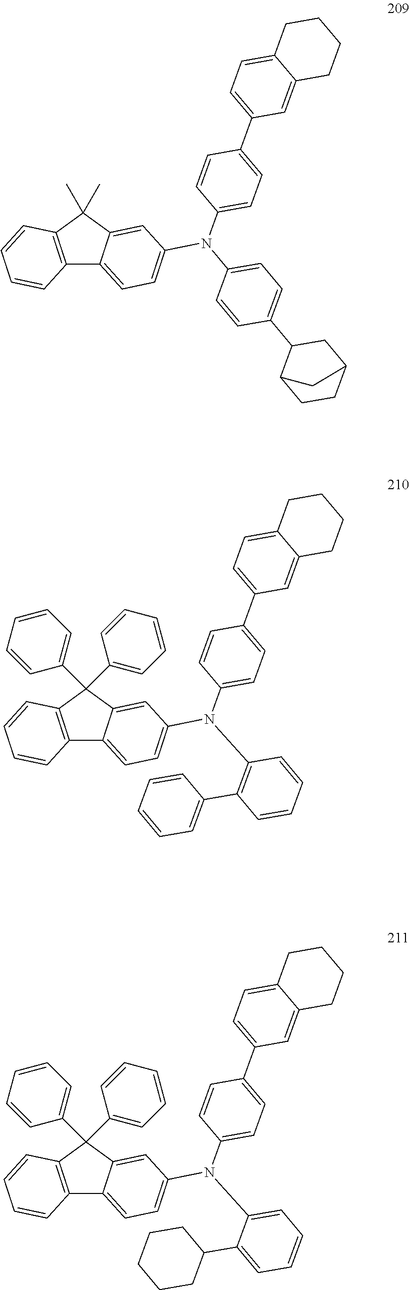 [SMILES: CC1(C)c2ccccc2-c2ccc(N(c3ccc(-c4ccc5c(c4)CCCC5)cc3)c3ccc(C4CC5CCC4C5)cc3)cc21 ; c1ccc(-c2ccccc2N(c2ccc(-c3ccc4c(c3)CCCC4)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccc6c(c5)CCCC6)cc4)c4ccccc4C4CCCCC4)cc32)cc1]

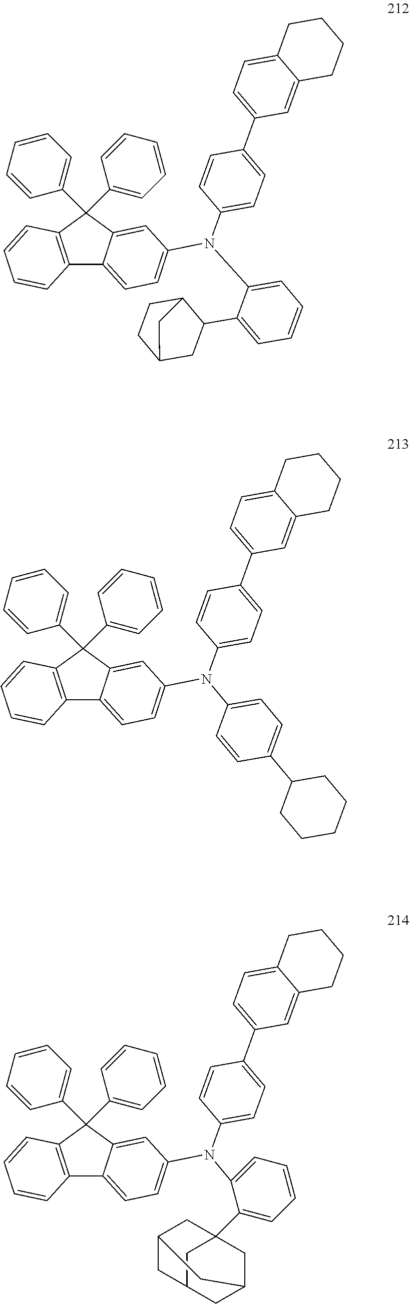 [SMILES: c1ccc(C2(c3ccccc3)c3ccccc3-c3ccc(N(c4ccc(-c5ccc6c(c5)CCCC6)cc4)c4ccccc4C4CC5CCC4C5)cc32)cc1 ; c1ccc(C2(c3ccccc3)c3ccccc3-c3ccc(N(c4ccc(-c5ccc6c(c5)CCCC6)cc4)c4ccc(C5CCCCC5)cc4)cc32)cc1 ; c1ccc(C2(c3ccccc3)c3ccccc3-c3ccc(N(c4ccc(-c5ccc6c(c5)CCCC6)cc4)c4ccccc4C45CC6CC(CC(C6)C4)C5)cc32)cc1]

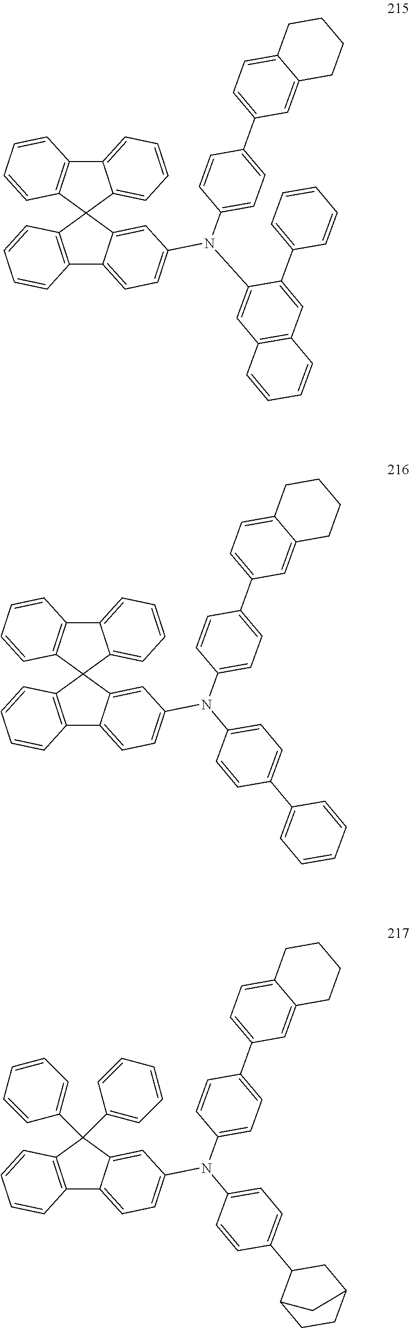 [SMILES: c1ccc(-c2cc3ccccc3cc2N(c2ccc(-c3ccc4c(c3)CCCC4)cc2)c2ccc3c(c2)C2(c4ccccc4-c4ccccc42)c2ccccc2-3)cc1 ; c1ccc(-c2ccc(N(c3ccc(-c4ccc5c(c4)CCCC5)cc3)c3ccc4c(c3)C3(c5ccccc5-c5ccccc53)c3ccccc3-4)cc2)cc1 ; c1ccc(C2(c3ccccc3)c3ccccc3-c3ccc(N(c4ccc(-c5ccc6c(c5)CCCC6)cc4)c4ccc(C5CC6CCC5C6)cc4)cc32)cc1]

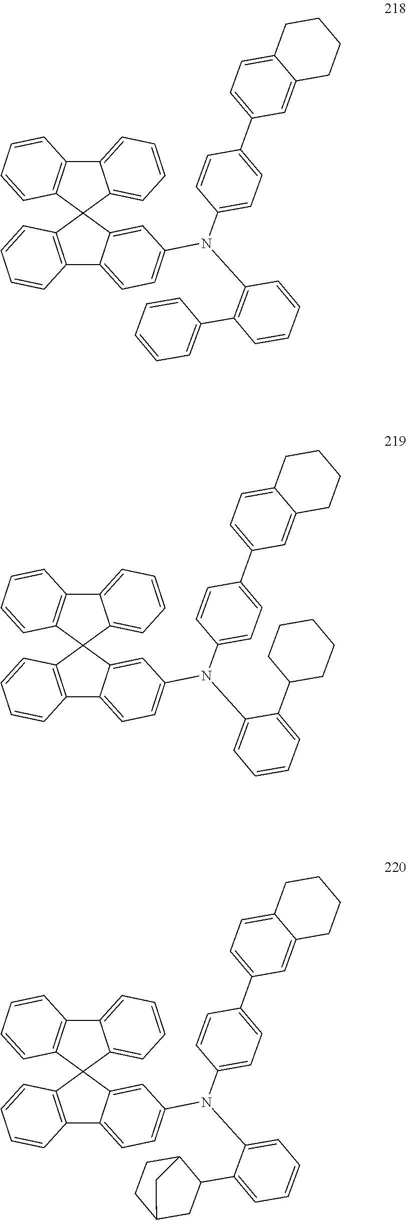 [SMILES: c1ccc(-c2ccccc2N(c2ccc(-c3ccc4c(c3)CCCC4)cc2)c2ccc3c(c2)C2(c4ccccc4-c4ccccc42)c2ccccc2-3)cc1 ; c1ccc2c(c1)-c1ccccc1C21c2ccccc2-c2ccc(N(c3ccc(-c4ccc5c(c4)CCCC5)cc3)c3ccccc3C3CCCCC3)cc21 ; c1ccc2c(c1)-c1ccccc1C21c2ccccc2-c2ccc(N(c3ccc(-c4ccc5c(c4)CCCC5)cc3)c3ccccc3C3CC4CCC3C4)cc21]

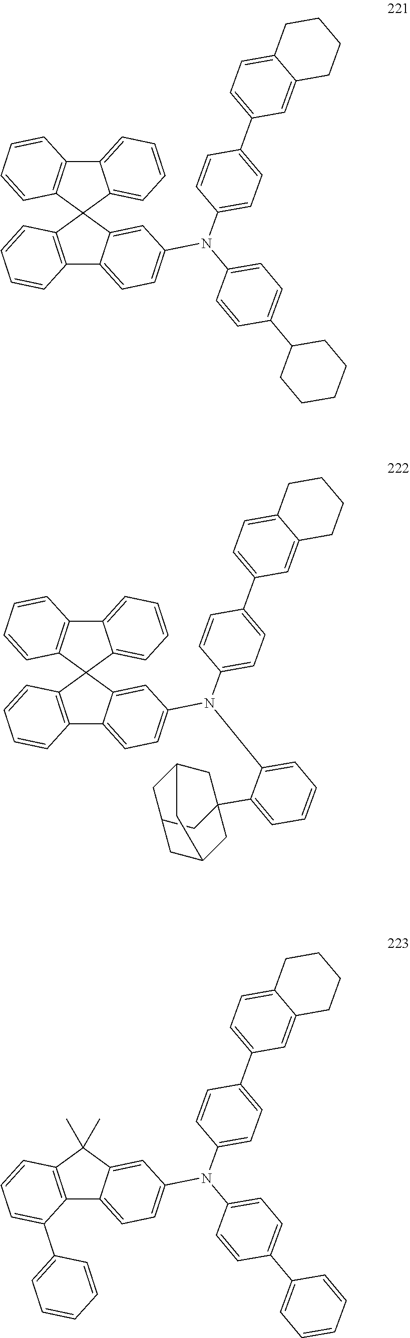 [SMILES: c1ccc2c(c1)-c1ccccc1C21c2ccccc2-c2ccc(N(c3ccc(-c4ccc5c(c4)CCCC5)cc3)c3ccc(C4CCCCC4)cc3)cc21 ; c1ccc2c(c1)-c1ccccc1C21c2ccccc2-c2ccc(N(c3ccc(-c4ccc5c(c4)CCCC5)cc3)c3ccccc3C34CC5CC(CC(C5)C3)C4)cc21 ; CC1(C)c2cc(N(c3ccc(-c4ccccc4)cc3)c3ccc(-c4ccc5c(c4)CCCC5)cc3)ccc2-c2c(-c3ccccc3)cccc21]

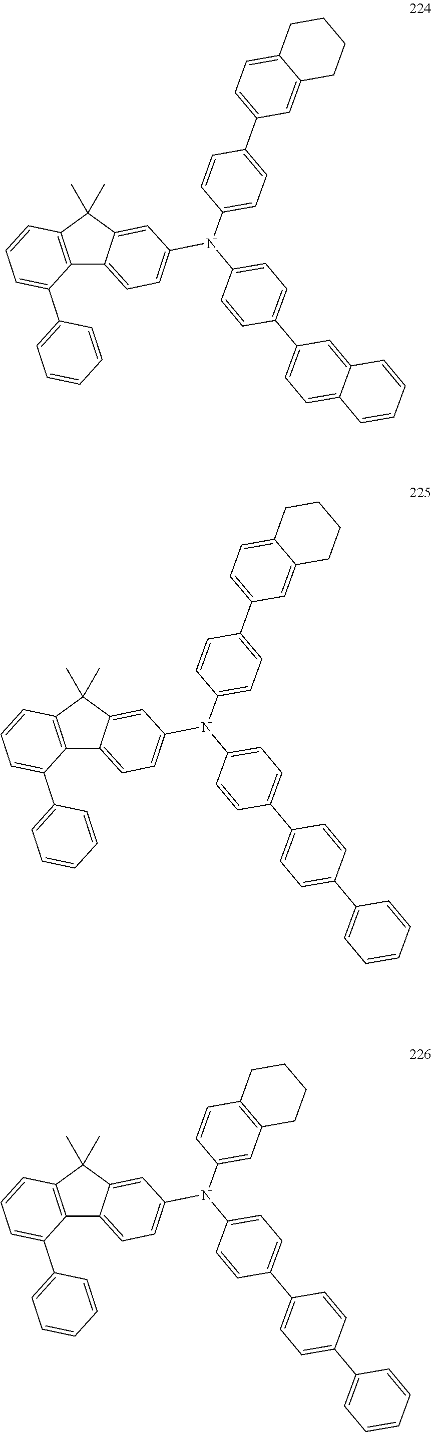 [SMILES: CC1(C)c2cc(N(c3ccc(-c4ccc5c(c4)CCCC5)cc3)c3ccc(-c4ccc5ccccc5c4)cc3)ccc2-c2c(-c3ccccc3)cccc21 ; CC1(C)c2cc(N(c3ccc(-c4ccc(-c5ccccc5)cc4)cc3)c3ccc(-c4ccc5c(c4)CCCC5)cc3)ccc2-c2c(-c3ccccc3)cccc21 ; CC1(C)c2cc(N(c3ccc(-c4ccc(-c5ccccc5)cc4)cc3)c3ccc4c(c3)CCCC4)ccc2-c2c(-c3ccccc3)cccc21]

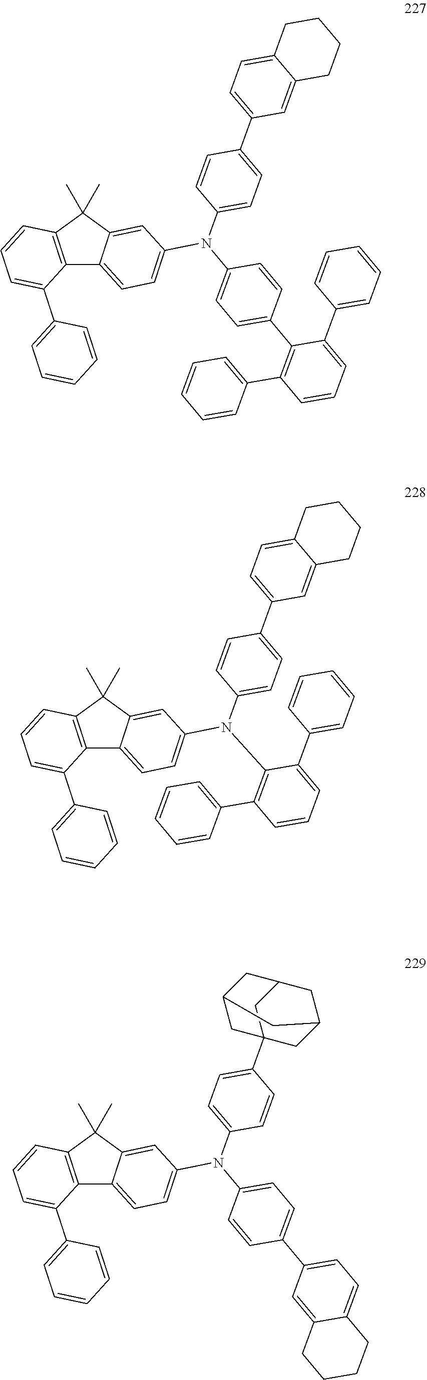 [SMILES: CC1(C)c2cc(N(c3ccc(-c4ccc5c(c4)CCCC5)cc3)c3ccc(-c4c(-c5ccccc5)cccc4-c4ccccc4)cc3)ccc2-c2c(-c3ccccc3)cccc21 ; CC1(C)c2cc(N(c3ccc(-c4ccc5c(c4)CCCC5)cc3)c3c(-c4ccccc4)cccc3-c3ccccc3)ccc2-c2c(-c3ccccc3)cccc21 ; CC1(C)c2cc(N(c3ccc(-c4ccc5c(c4)CCCC5)cc3)c3ccc(C45CC6CC(CC(C6)C4)C5)cc3)ccc2-c2c(-c3ccccc3)cccc21]

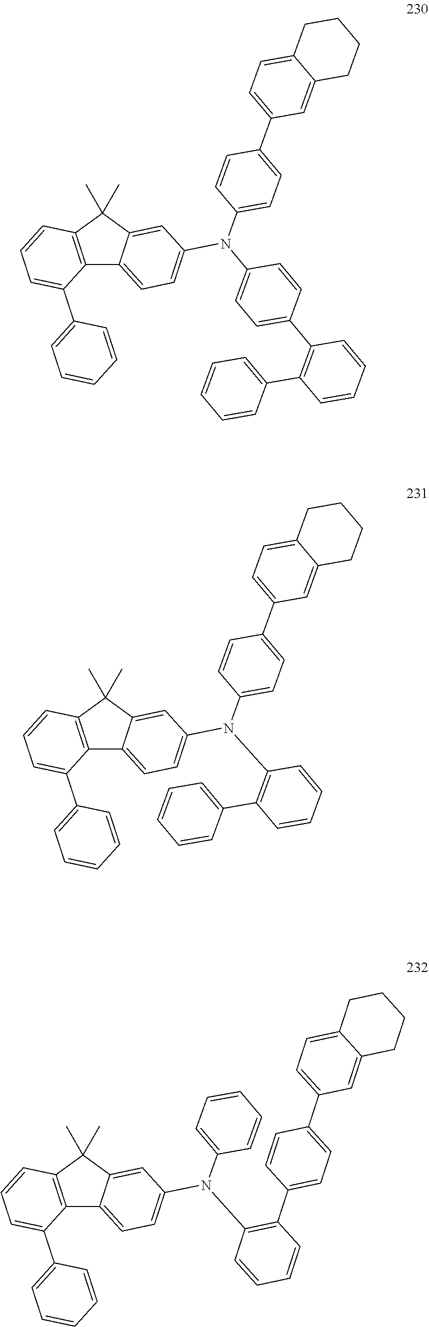 [SMILES: CC1(C)c2cc(N(c3ccc(-c4ccc5c(c4)CCCC5)cc3)c3ccc(-c4ccccc4-c4ccccc4)cc3)ccc2-c2c(-c3ccccc3)cccc21 ; CC1(C)c2cc(N(c3ccc(-c4ccc5c(c4)CCCC5)cc3)c3ccccc3-c3ccccc3)ccc2-c2c(-c3ccccc3)cccc21 ; CC1(C)c2cc(N(c3ccccc3)c3ccccc3-c3ccc(-c4ccc5c(c4)CCCC5)cc3)ccc2-c2c(-c3ccccc3)cccc21]

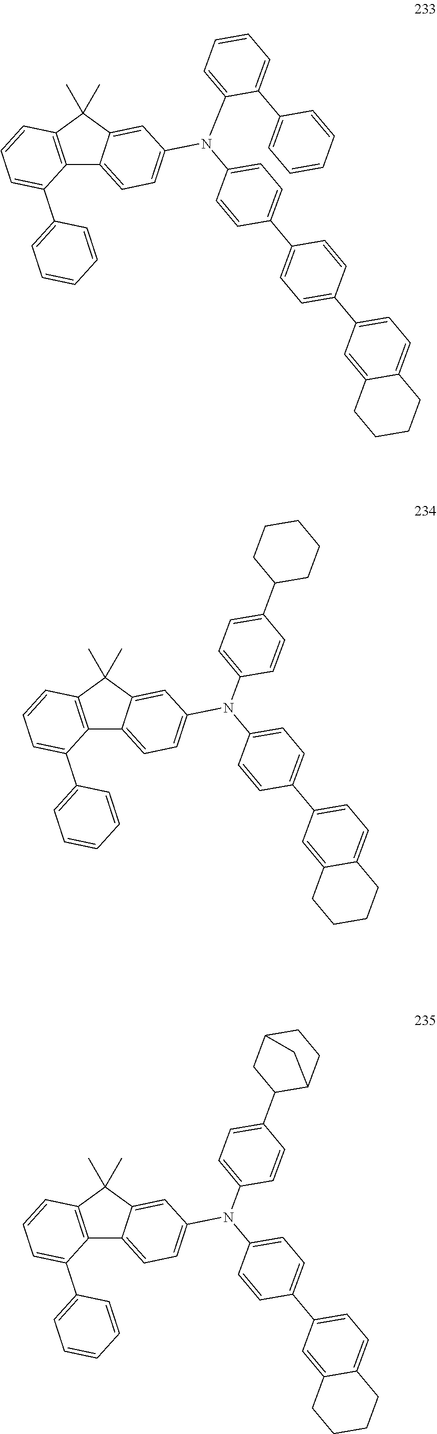 [SMILES: CC1(C)c2cc(N(c3ccc(-c4ccc(-c5ccc6c(c5)CCCC6)cc4)cc3)c3ccccc3-c3ccccc3)ccc2-c2c(-c3ccccc3)cccc21 ; CC1(C)c2cc(N(c3ccc(-c4ccc5c(c4)CCCC5)cc3)c3ccc(C4CCCCC4)cc3)ccc2-c2c(-c3ccccc3)cccc21 ; CC1(C)c2cc(N(c3ccc(-c4ccc5c(c4)CCCC5)cc3)c3ccc(C4CC5CCC4C5)cc3)ccc2-c2c(-c3ccccc3)cccc21]

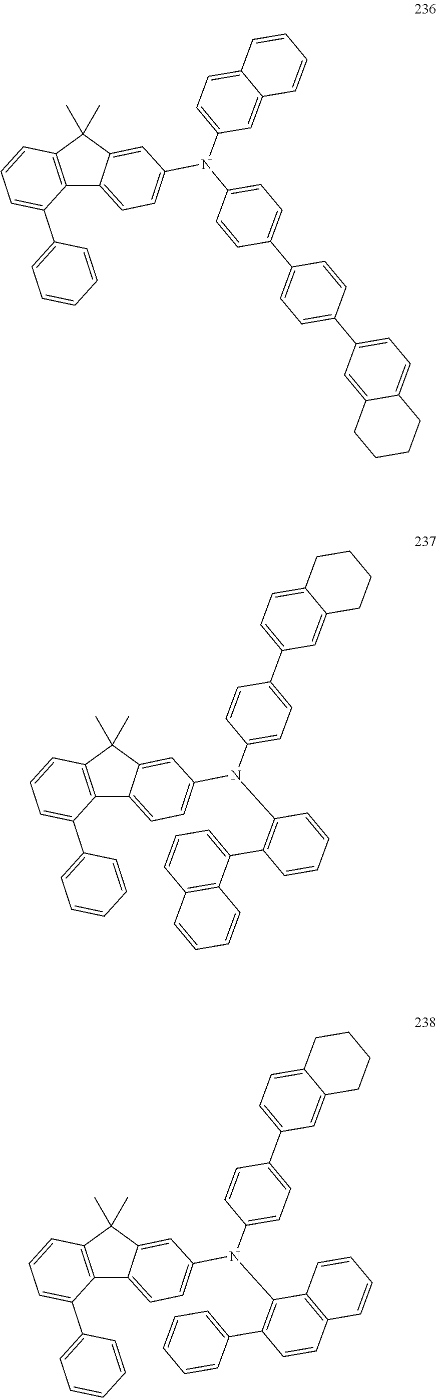 [SMILES: CC1(C)c2cc(N(c3ccc(-c4ccc(-c5ccc6c(c5)CCCC6)cc4)cc3)c3ccc4ccccc4c3)ccc2-c2c(-c3ccccc3)cccc21 ; CC1(C)c2cc(N(c3ccc(-c4ccc5c(c4)CCCC5)cc3)c3ccccc3-c3cccc4ccccc34)ccc2-c2c(-c3ccccc3)cccc21 ; CC1(C)c2cc(N(c3ccc(-c4ccc5c(c4)CCCC5)cc3)c3c(-c4ccccc4)ccc4ccccc34)ccc2-c2c(-c3ccccc3)cccc21]

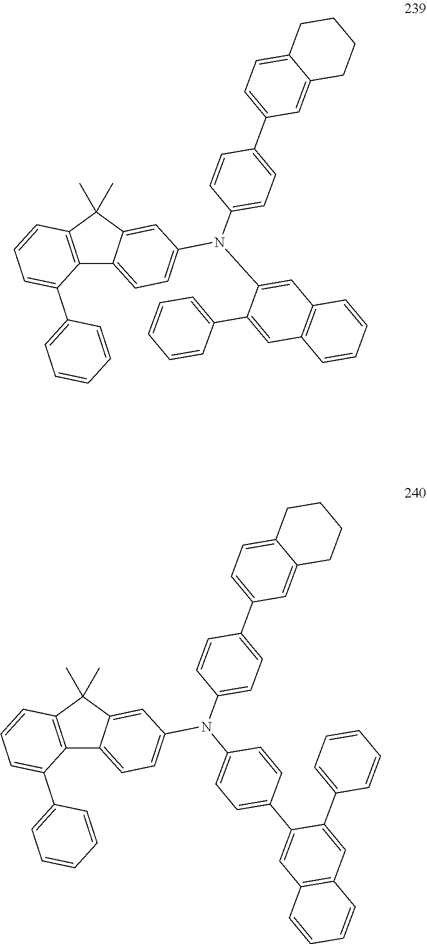 [SMILES: CC1(C)c2cc(N(c3ccc(-c4ccc5c(c4)CCCC5)cc3)c3cc4ccccc4cc3-c3ccccc3)ccc2-c2c(-c3ccccc3)cccc21 ; CC1(C)c2cc(N(c3ccc(-c4ccc5c(c4)CCCC5)cc3)c3ccc(-c4cc5ccccc5cc4-c4ccccc4)cc3)ccc2-c2c(-c3ccccc3)cccc21]

The amine compound of one or more embodiments may include one fluorene moiety and one tetrahydronaphthyl group, bonded to an amine group. The amine compound of one or more embodiments may be a monoamine compound. The amine compound of one or more embodiments does not include (e.g., may exclude) a substituted or unsubstituted carbazole group, a substituted or unsubstituted anthracenyl group, a substituted or unsubstituted phenanthryl group, a substituted or unsubstituted pyrenyl group, and a substituted or unsubstituted triphenylenyl group. The anthracenyl group, phenanthryl group, pyrenyl group, and triphenylenyl group are substituents formed by fusing three or more phenyl groups. When compared to the compound including a substituent formed by fusing three or more phenyl groups (in place of, for example, the phenyl group in Formula 1), the amine compound of one or more embodiments may show improved difference between the energy level of the highest occupied molecular orbital (HOMO) and the energy level of the lowest unoccupied molecular orbital (LUMO), and may improve charge mobility, thereby improving the driving properties of the light emitting element ED.

The amine compound of one or more embodiments, including one fluorene moiety and one tetrahydronaphthyl group may have high glass transition temperature and may prevent or reduce crystallization. In addition, the amine compound of one or more embodiments, including one fluorene moiety and one tetrahydronaphthyl group may show a low refractive index and excellent or improved hole transport properties. Accordingly, the light emitting element ED including the amine compound of one or more embodiments may show a reduced driving voltage, high luminance, high efficiency and long-life characteristics. For example, in the light emitting element ED of one or more embodiments, the hole transport region HTR may include the amine compound of one or more embodiments.

The hole transport region HTR is provided on the first electrode EL1. The hole transport region HTR may include at least one of a hole injection layer HIL, a hole transport layer HTL, a buffer layer or an emission auxiliary layer, and/or an electron blocking layer EBL. At least one of the hole injection layer HIL, the hole transport layer HTL and/or the electron blocking layer EBL may include the amine compound of one or more embodiments.

The hole transport region HTR may have a single layer formed using a single material, a single layer formed using multiple different materials, or a multilayer structure including multiple layers formed using multiple different materials. For example, the hole transport region HTR may have the structure of a single layer of a hole injection layer HIL or a hole transport layer HTL, and may have a structure of a single layer formed using a hole injection material and a hole transport material.

In some embodiments, the hole transport region HTR may have a structure of a single layer formed using multiple different materials, or a structure stacked from the first electrode EL1 of hole injection layer HIL/hole transport layer HTL, hole injection layer HIL/first hole transport layer HTL-1/second hole transport layer HTL-2/third hole transport layer HTL-3, hole injection layer HIL/hole transport layer HTL/buffer layer, hole injection layer HIL/buffer layer, hole transport layer HTL/buffer layer, or hole injection layer HIL/hole transport layer HTL/electron blocking layer EBL, without limitation.

If the hole transport region HTR includes the first to third hole transport layers HTL-1, HTL-2 and HTL-3, at least one of the first to third hole transport layers HTL-1, HTL-2 and HTL-3 may include the amine compound of one or more embodiments. For example, the first to third hole transport layers HTL-1, HTL-2 and HTL-3 may include the amine compound of one or more embodiments. The first and third hole transport layers HTL-1 and HTL-3 may include the same material, and the second hole transport layer HTL-2 may include a different material from that of the first and third hole transport layers HTL-1 and HTL-3. However, these are illustrations, and one or more embodiments of the present disclosure is not limited thereto.

The hole transport region HTR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may further include a compound represented below. The hole transport region HTR may include a compound represented by Formula H-1 below:

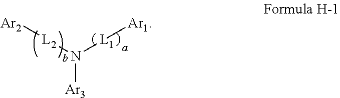

In Formula H-1 above, L₁ and L₂ may be each independently a direct linkage, a substituted or unsubstituted arylene group of 6 to 30 ring-forming carbon atoms, or a substituted or unsubstituted heteroarylene group of 2 to 30 ring-forming carbon atoms. “a” and “b” may be each independently an integer of 0 to 10. In one or more embodiments, if “a” or “b” is an integer of 2 or more, multiple L₁ and L₂ may be each independently a substituted or unsubstituted arylene group of 6 to 30 ring-forming carbon atoms, or a substituted or unsubstituted heteroarylene group of 2 to 30 ring-forming carbon atoms.

In Formula H-1, Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. In Formula H-1, Ar₃ may be a substituted or unsubstituted aryl group of 6 to 30 ring-forming carbon atoms.

The compound represented by Formula H-1 may be a monoamine compound. In one or more embodiments, the compound represented by Formula H-1 may be a diamine compound in which at least one of Ar₁ to Ar₃ includes an amine group as a substituent. In one or more embodiments, the compound represented by Formula H-1 may be a carbazole-based compound in which at least one of Ar₁ and/or Ar₂ includes a substituted or unsubstituted carbazole group, or a fluorene-based compound in which at least one of Ar₁ and/or Ar₂ includes a substituted or unsubstituted fluorene group.

The compound represented by Formula H-1 may be represented by any one of the compounds in Compound Group H below. However, the compounds listed in Compound Group H are only illustrations, and the compound represented by Formula H-1 is not limited to the compounds represented in Compound Group H below.

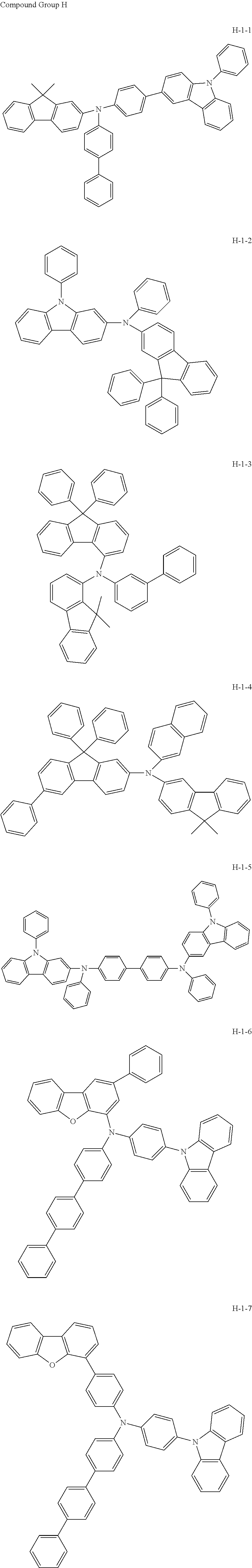

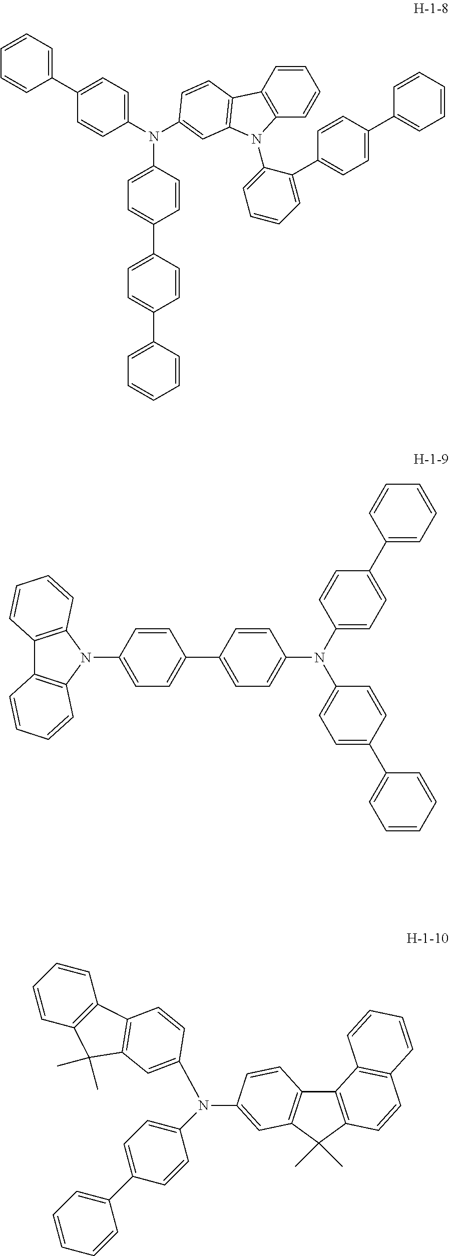 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

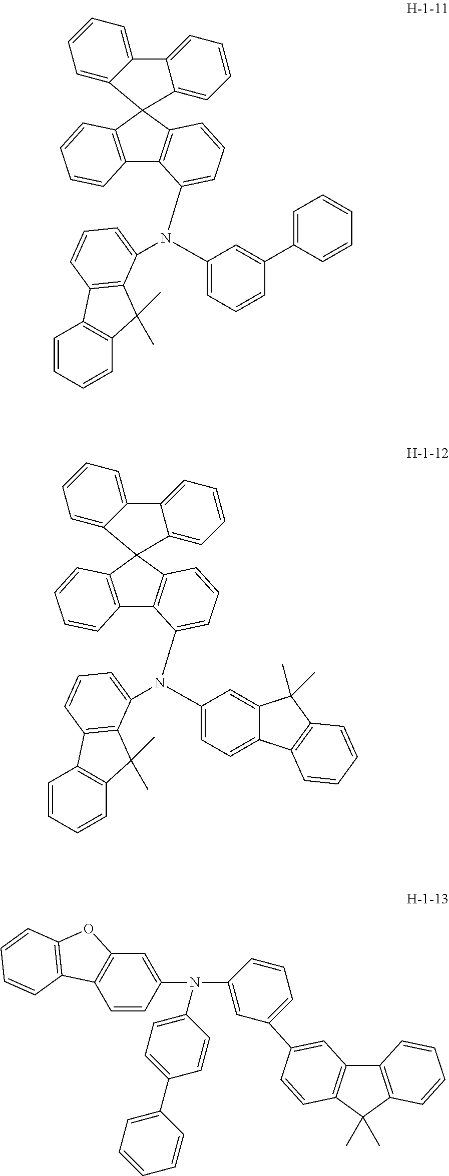 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

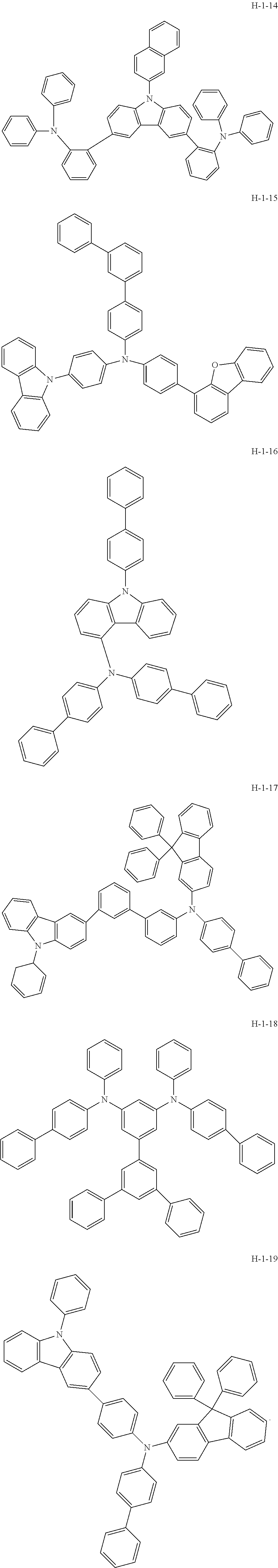

The hole transport region HTR may include a phthalocyanine compound (such as copper phthalocyanine), N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], and/or dipyrazino[2,3-f:2′,3′-h] quinoxaline-2,3,6,7,10,11-hexacarbonitrile (HATCN).

In one or more embodiments, the hole transport region HTR may include carbazole derivatives (such as N-phenyl carbazole and/or polyvinyl carbazole), fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazolyl)benzene (mCP), and/or 1,3-bis(1,8-dimethyl-9H-carbazol-9-yl)benzene (mDCP), etc.

The hole transport region HTR may include the compounds of the hole transport region in at least one of the hole injection layer HIL, hole transport layer HTL, and/or electron blocking layer EBL.

The thickness of the hole transport region HTR may be, for example, from about 50 Å to about 15,000 Å. The thickness of the hole transport region HTR may be from about 100 Å to about 10,000 Å, for example, from about 100 Å to about 5,000 Å.

In case where the hole transport region HTR includes a hole injection layer HIL, the thickness of the hole injection layer HIL may be, for example, from about 30 Å to about 1,000 Å. In case where the hole transport region HTR includes a hole transport layer HTL, the thickness of the hole transport layer HTL may be from about 30 Å to about 1,000 Å. For example, in case where the hole transport region HTR includes an electron blocking layer EBL, the thickness of the electron blocking layer EBL may be from about 10 Å to about 1,000 Å. If the thicknesses of the hole transport region HTR, the hole injection layer HIL, the hole transport layer HTL and/or the electron blocking layer EBL satisfy their respective above-described ranges, satisfactory or suitable hole transport properties may be achieved without substantial increase of a driving voltage.

The hole transport region HTR may further include a charge generating material to increase conductivity, in addition to the above-described materials. The charge generating material may be dispersed substantially uniformly or non-substantially uniformly in the hole transport region HTR. The charge generating material may be, for example, a p-dopant. The p-dopant may include at least one of metal halide compounds, quinone derivatives, metal oxides, and/or cyano group-containing compounds, without limitation. For example, the p-dopant may include metal halide compounds (such as CuI and/or RbI), quinone derivatives (such as tetracyanoquinodimethane (TCNQ) and/or 2,3,5,6-tetrafluoro-7,7,8,8-tetracyanoquinodimethane (F4-TCNQ)), metal oxides (such as tungsten oxide and/or molybdenum oxide), cyano group-containing compounds (such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9)), etc., without limitation.

As described above, the hole transport region HTR may further include at least one of a buffer layer and/or an electron blocking layer EBL, in addition to the hole injection layer HIL and the hole transport layer HTL. The buffer layer may compensate resonance distance according to the wavelength of light emitted from an emission layer EML and may increase emission efficiency. As materials included in the buffer layer, materials which may be included in the hole transport region HTR may be used. The electron blocking layer EBL is a layer playing the role of preventing or reducing the injection of electrons from the electron transport region ETR to the hole transport region HTR.

Referring to FIG. 3 to FIG. 7 again, the first electrode EL1 has conductivity. The first electrode EL1 may be formed using a metal material, a metal alloy or a suitable conductive compound. The first electrode EL1 may be an anode or a cathode. However, one or more embodiments of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one of Ag, Mg, Cu, Al, Pt, Pd, Au, Ni, Nd, Ir, Cr, Li, Ca, LiF, Mo, Ti, W, In, Sn, Zn, a compound of two or more thereof, a mixture of two or more thereof, and/or an oxide thereof.

If the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, one or more compounds thereof, or one or more mixtures thereof (for example, a mixture of Ag and Mg). Also, the first electrode EL1 may have a structure including multiple layers including a reflective layer or a transflective layer formed using any of the above materials, and a transmissive conductive layer formed using ITO, IZO, ZnO, and/or ITZO. For example, the first electrode EL1 may have a three-layer structure of ITO/Ag/ITO. In one or more embodiments, the first electrode EL1 may include one or more of the above-described metal materials, combinations of two or more metal materials selected from the above-described metal materials, and/or oxides of the above-described metal materials. However, one or more embodiments of the present disclosure is not limited thereto. The thickness of the first electrode EL1 may be from about 700 Å to about 10,000 Å. For example, the thickness of the first electrode EL1 may be from about 1,000 Å to about 3,000 Å.

The emission layer EML is provided on the hole transport region HTR. The emission layer EML may have a thickness of, for example, about 100 Å to about 1,000 Å or about 100 Å to about 300 Å. The emission layer EML may have a single layer formed using a single material, a single layer formed using multiple different materials, or a multilayer structure having multiple layers formed using multiple different materials.

In the light emitting element ED of one or more embodiments, the emission layer EML may include one or more of anthracene derivatives, pyrene derivatives, fluoranthene derivatives, chrysene derivatives, dihydrobenzanthracene derivatives, and/or triphenylene derivatives. For example, the emission layer EML may include anthracene derivatives and/or pyrene derivatives.

The emission layer EML may include a compound represented by Formula E-1 below. The compound represented by Formula E-1 below may be used as a fluorescence host material:

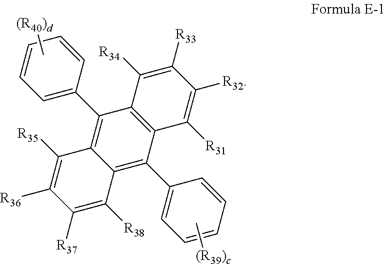

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In one or more embodiments, R₃₁ to R₄₀ may be combined with an adjacent group to form a saturated hydrocarbon ring, an unsaturated hydrocarbon ring, a saturated heterocycle, or an unsaturated heterocycle. In Formula E-1, “c” and “d” may be each independently an integer of 0 to 5.

Formula E-1 may be represented by any one of Compound E1 to Compound E-19 below:

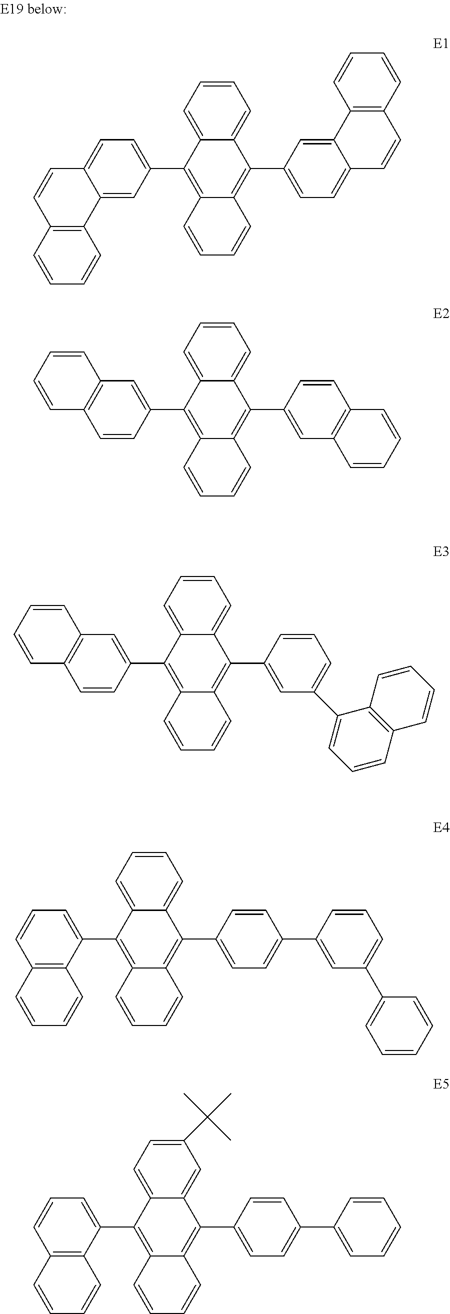

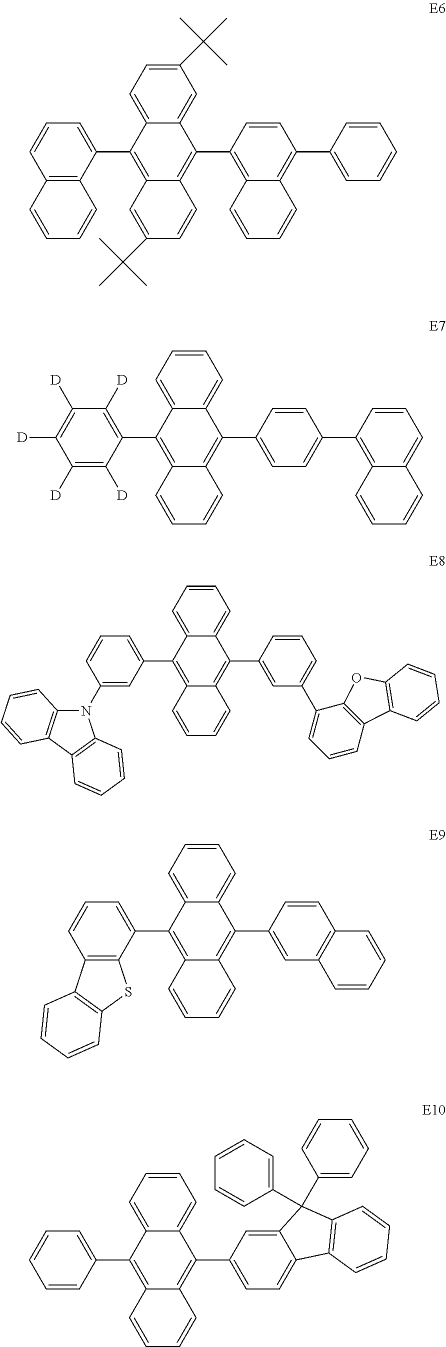

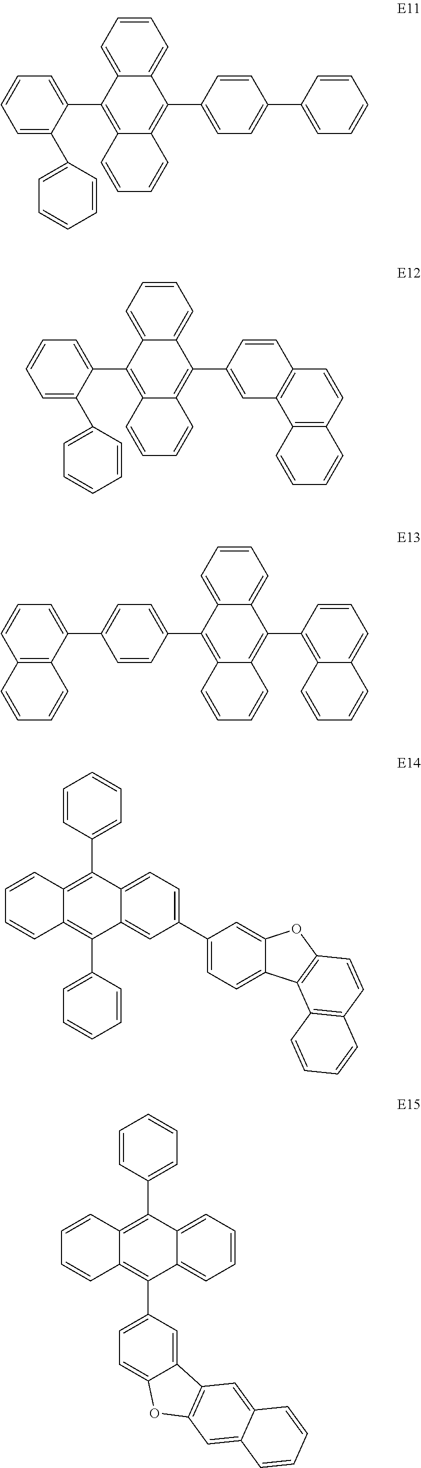

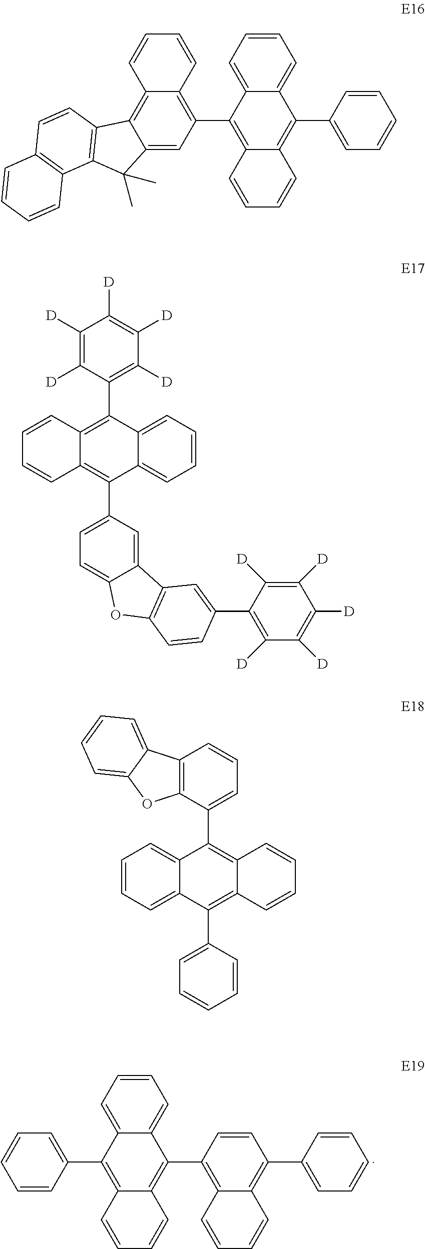

In the light emitting element ED of one or more embodiments, the emission layer EML may include a host and a dopant. For example, the emission layer EML may include one host and one dopant. In some embodiments, the emission layer EML may include two or more hosts, a sensitizer and a dopant. For example, the emission layer EML may include a hole transport host and an electron transport host. The emission layer EML may include a phosphorescence sensitizer or a thermally activated delayed fluorescence (TADF) sensitizer, as the sensitizer. If the emission layer EML includes a hole transport host, an electron transport host, a sensitizer and a dopant, the hole transport host and the electron transport host may form exciplexes, and energy transfer may occur from the exciplex to the sensitizer, and from the sensitizer to the dopant. However, this is an illustration, and the material included in the emission layer EML is not limited thereto.

In one or more embodiments, the emission layer EML may include at least one of a second compound represented by Formula HT-1, a third compound represented by Formula ET-1, and/or a fourth compound represented by Formula M-b below.

For example, the second compound may be used as the hole transport host material of the emission layer EML. The second compound represented by Formula HT-1 may include a carbazole moiety:

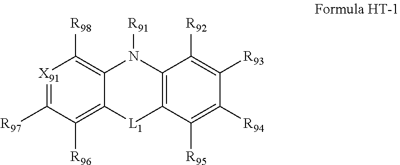

In Formula HT-1, L₁ may be a direct linkage, CR₉₉R₁₀₀, or SiR₁₀₁R₁₀₂. In Formula HT-1, X₉₁ may be N or CR₁₀₃. When L₁ is a direct linkage and X₉₁ is CR₁₀₃, the second compound represented by Formula HT-1 may include a carbazole group.

In Formula HT-1, R₉₁ to R₁₀₃ may each independently be a hydrogen atom, a deuterium atom, a cyano group, a substituted or unsubstituted silyl group, a substituted or unsubstituted oxy group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring.

The second compound may be represented by any one of the compounds in Compound Group 2 below. In Compound Group 2, D is a deuterium atom, and Ph is a phenyl group.

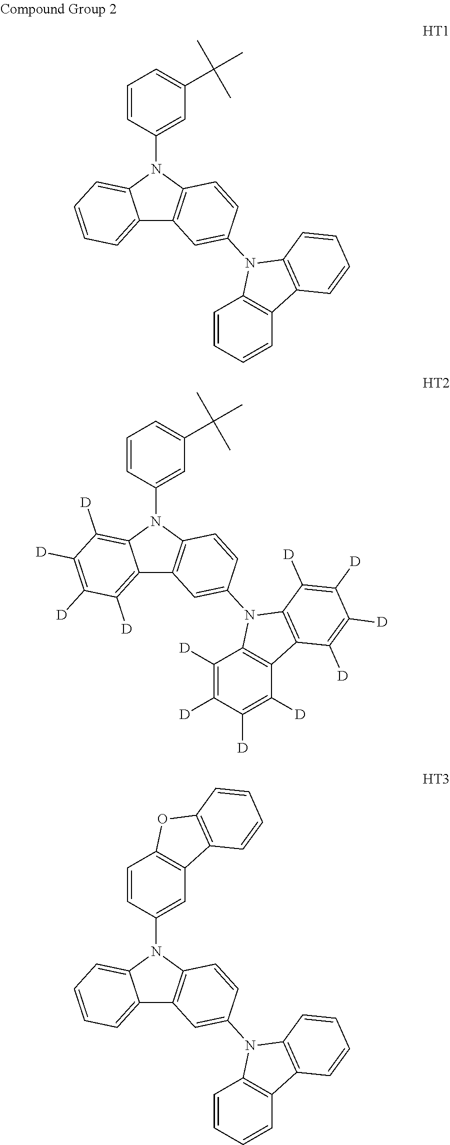

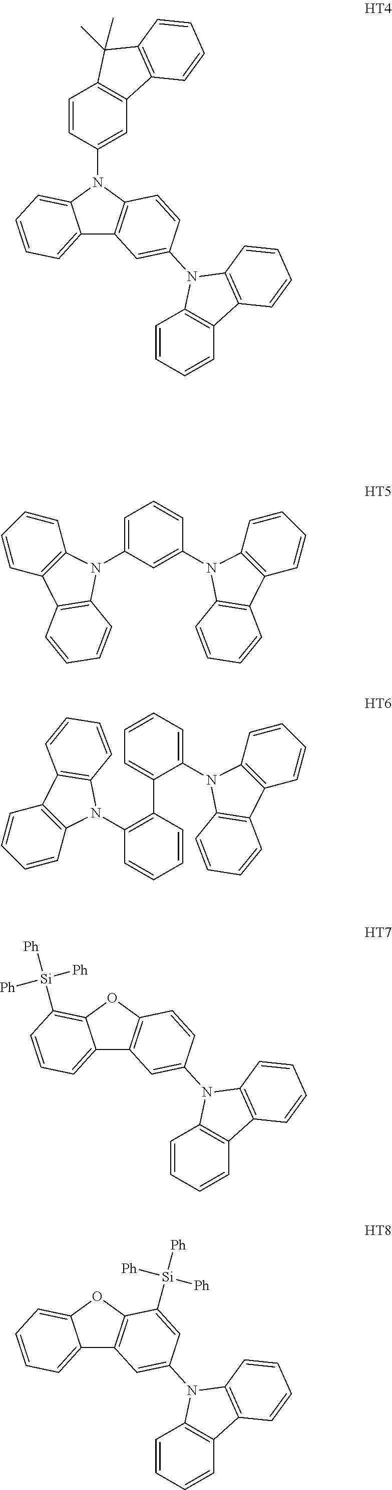 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

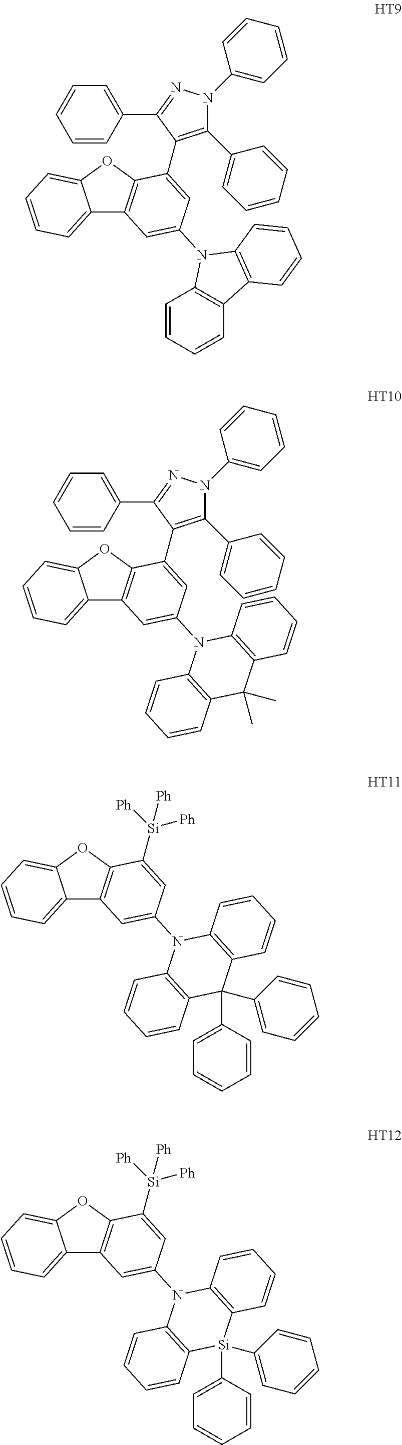 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

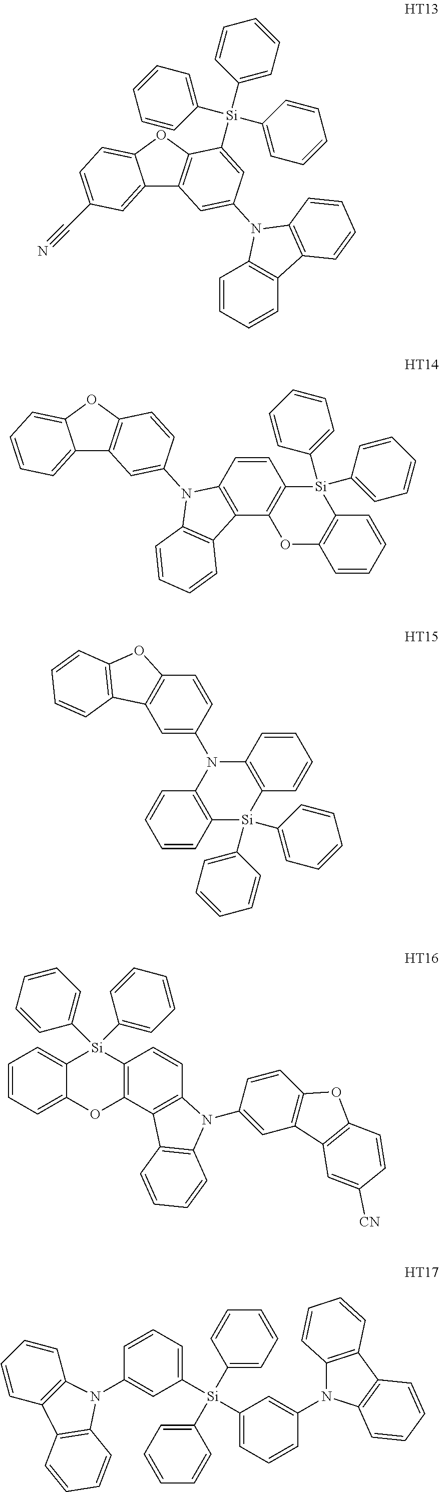 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1]

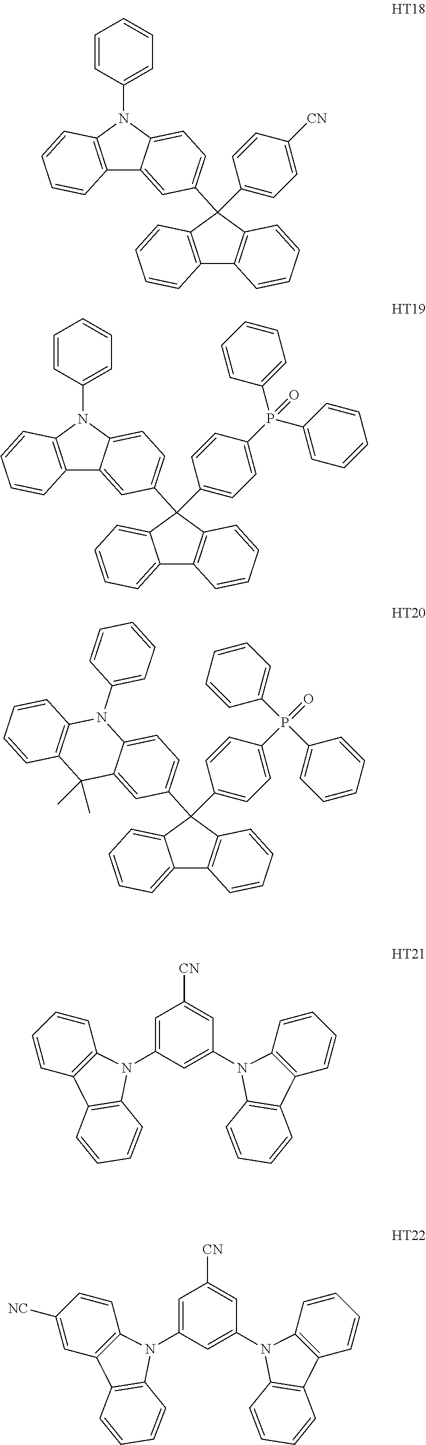

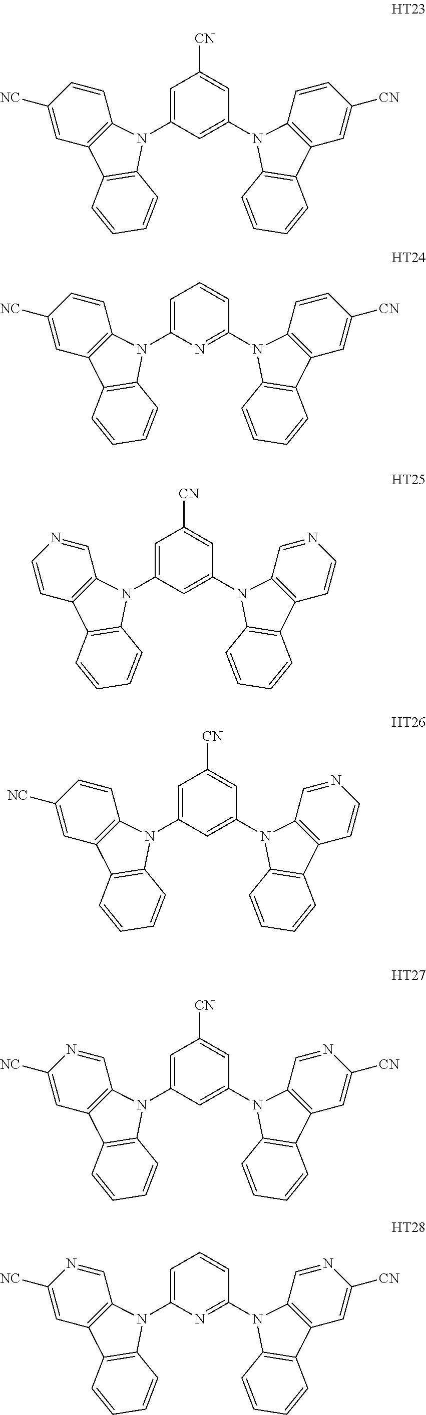 [SMILES: N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1]

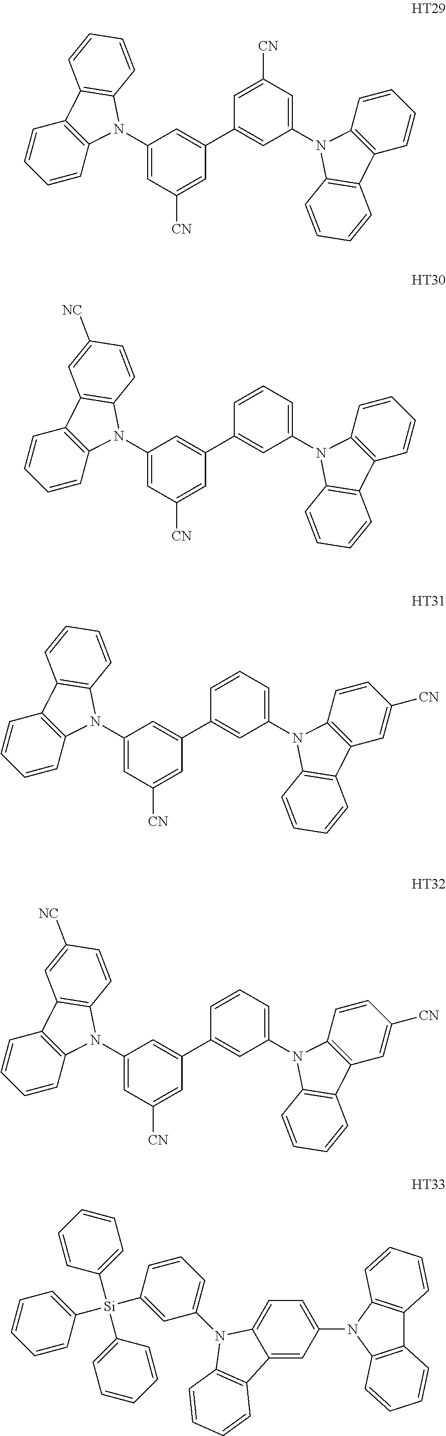 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1 ; c1ccc([Si](c2ccccc2)(c2ccccc2)c2cccc(-n3c4ccccc4c4cc(-n5c6ccccc6c6ccccc65)ccc43)c2)cc1]

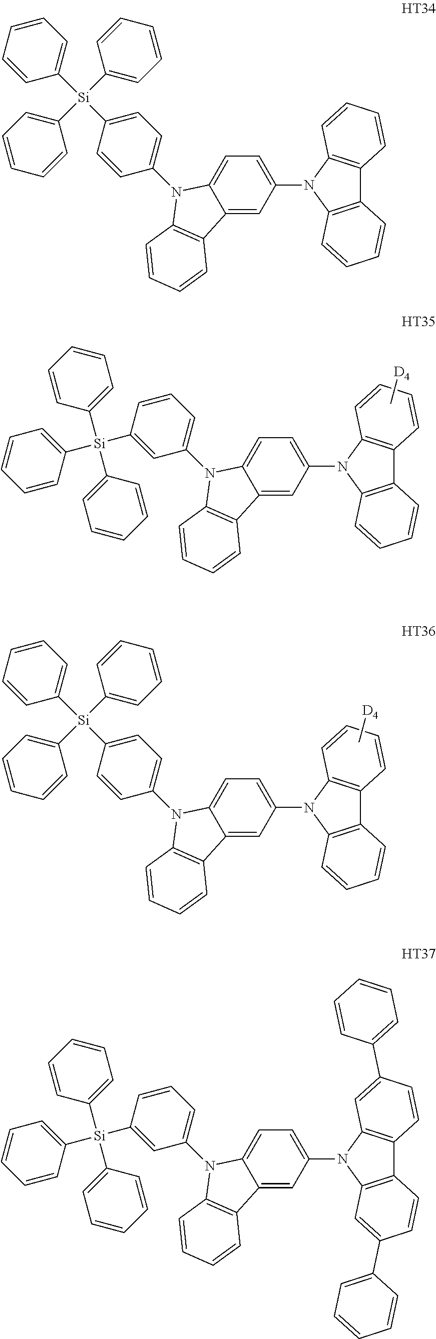

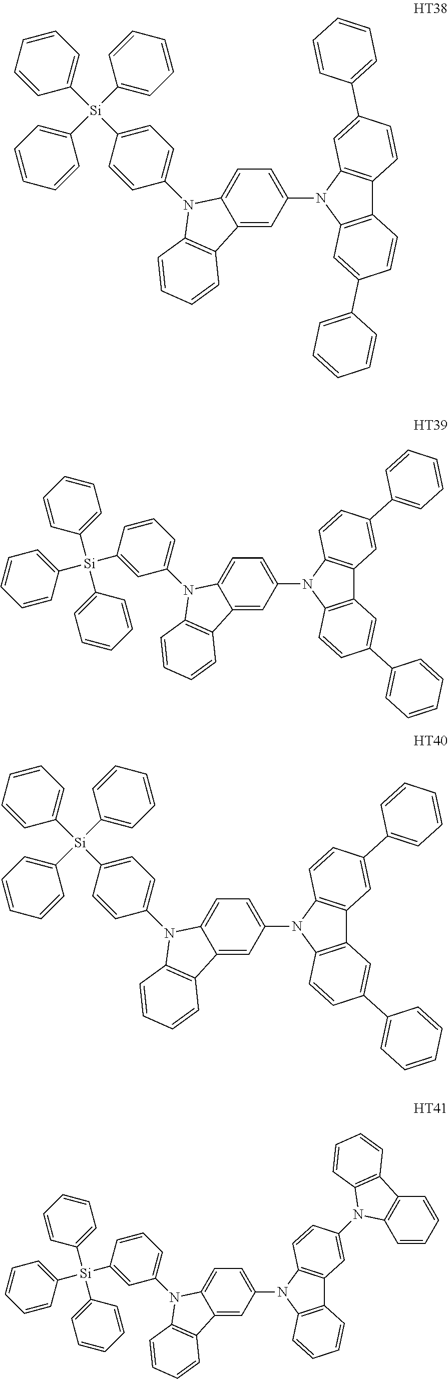 [SMILES: c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1 ; c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1]

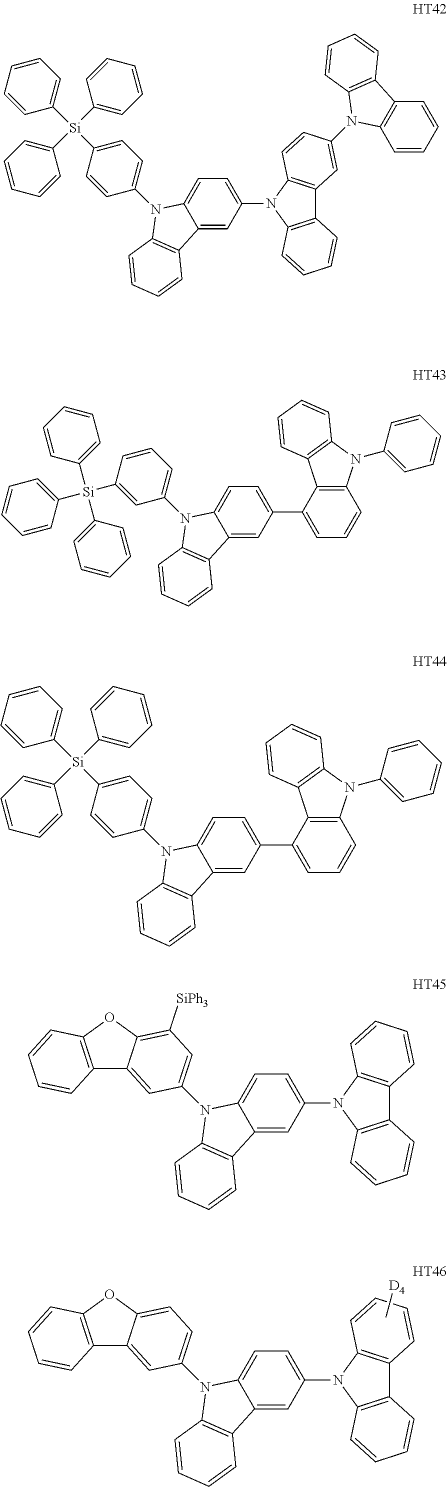

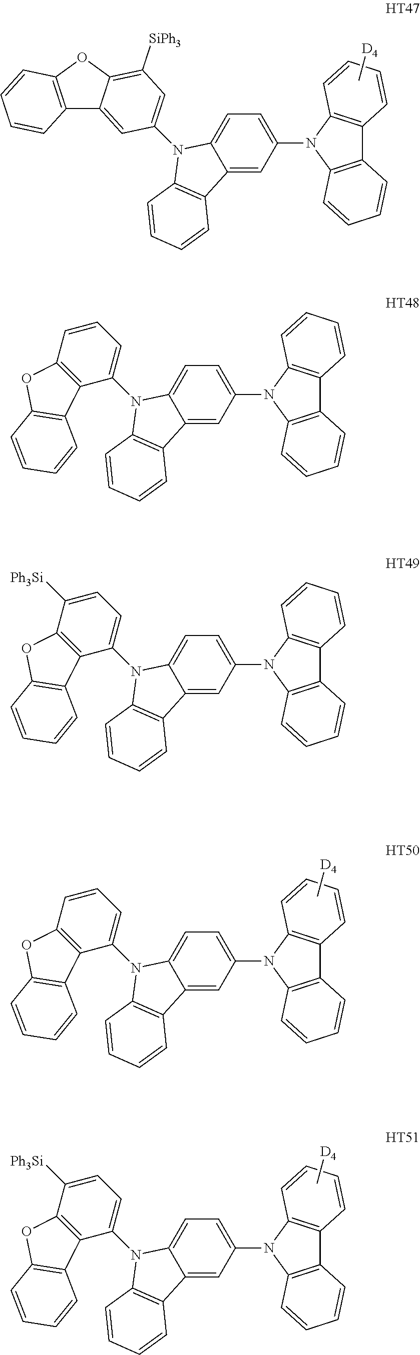

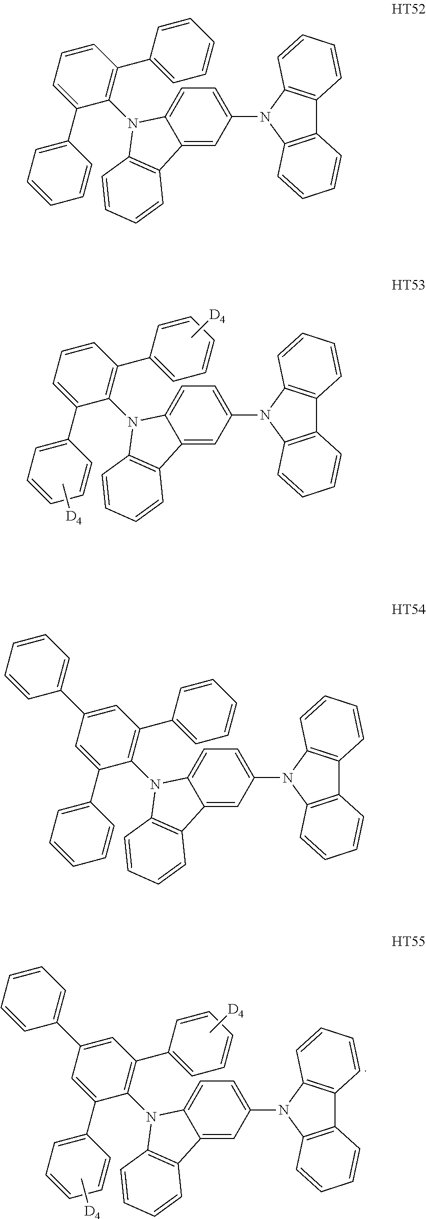

In one or more embodiments, the emission layer EML may include a third compound represented by Formula ET-1. The third compound represented by Formula ET-1 may include a heterocycle including N as a ring-forming atom. For example, the third compound may be used as the electron transport host material of the emission layer EML. In some embodiments, the third compound may be used as the electron transport material of the electron transport region ETR:

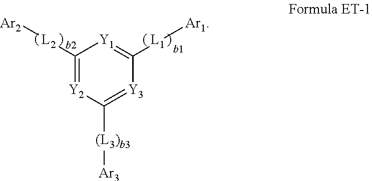

In Formula ET-1, at least one of Y₁ to Y₃ may be N, and the remainder thereof may be CRₐ, and Rₐ may be a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

b1 to b3 may be each independently an integer of 0 to 10. L₁ to L₃ may be each independently a direct linkage, a substituted or unsubstituted arylene group of 6 to 30 ring-forming carbon atoms, or a substituted or unsubstituted heteroarylene group of 2 to 30 ring-forming carbon atoms. If any of b1 to b3 are integers of 2 or more, L₁ to L₃ may be each independently a substituted or unsubstituted arylene group of 6 to 30 ring-forming carbon atoms, or a substituted or unsubstituted heteroarylene group of 2 to 30 ring-forming carbon atoms.

Ar₁ to Ar₃ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Ar₁ to Ar₃ may be substituted or unsubstituted phenyl groups, or substituted or unsubstituted carbazole groups.

The third compound may be represented by any one of the compounds in Compound Group 3 below. The light emitting element ED of one or more embodiments may include any one of the compounds in Compound Group 3. In Compound Group 3, D is a deuterium atom, and Ph is a phenyl group.

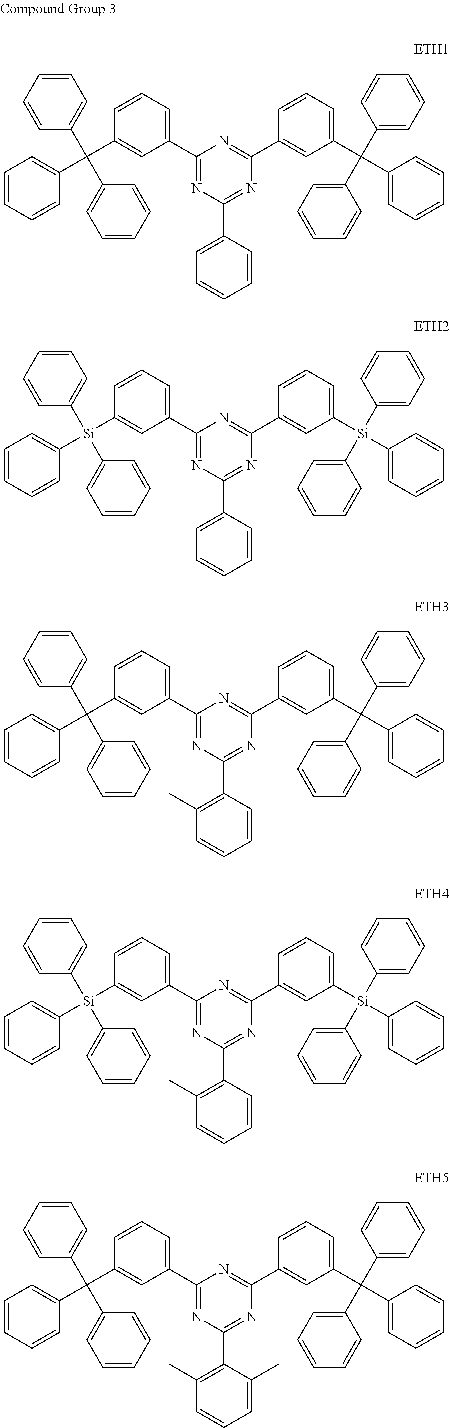

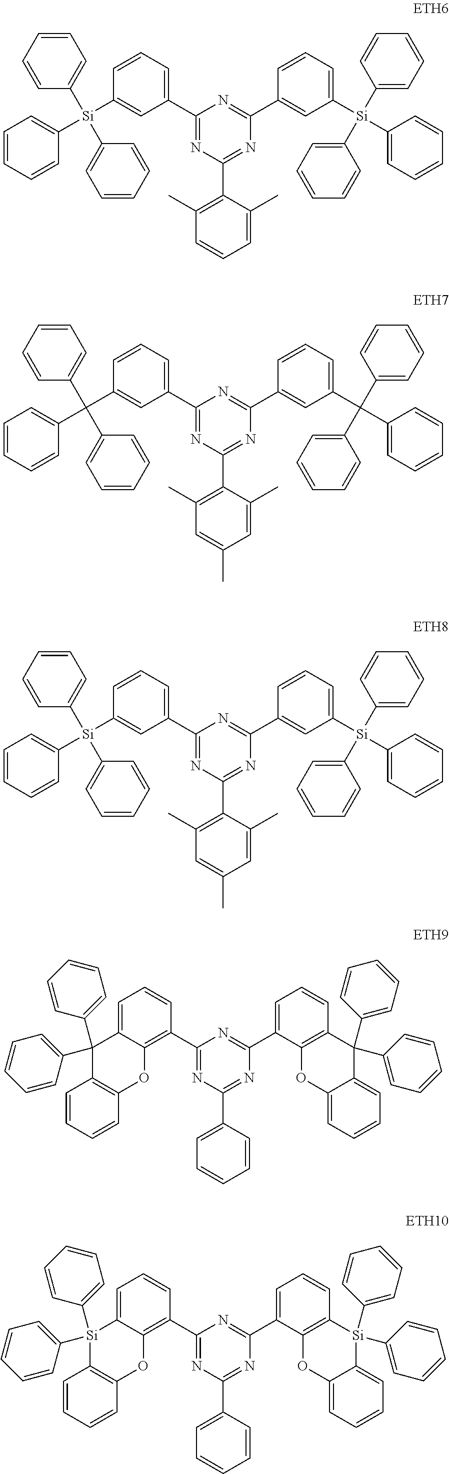

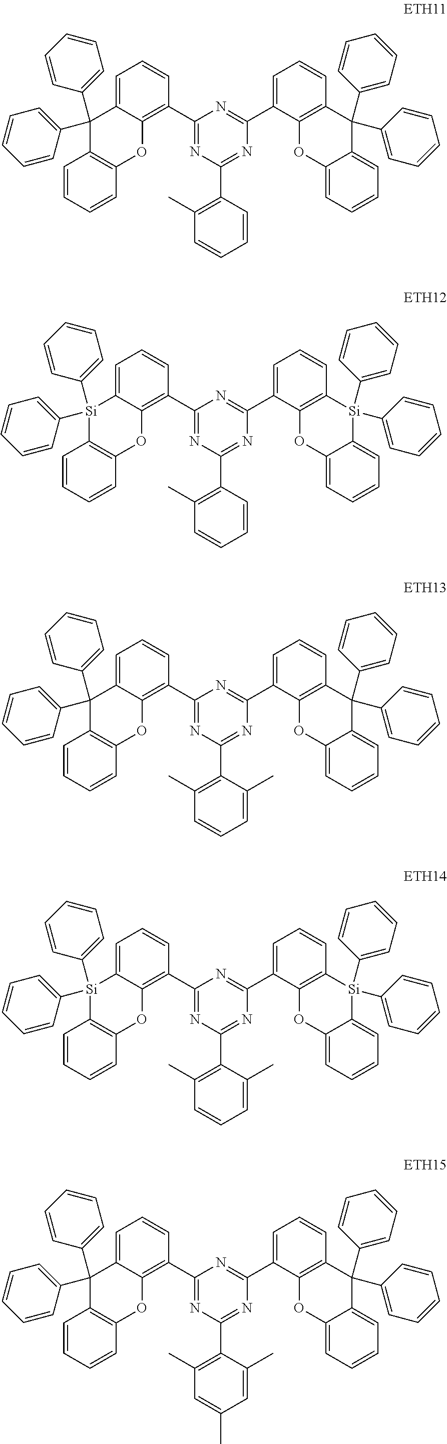

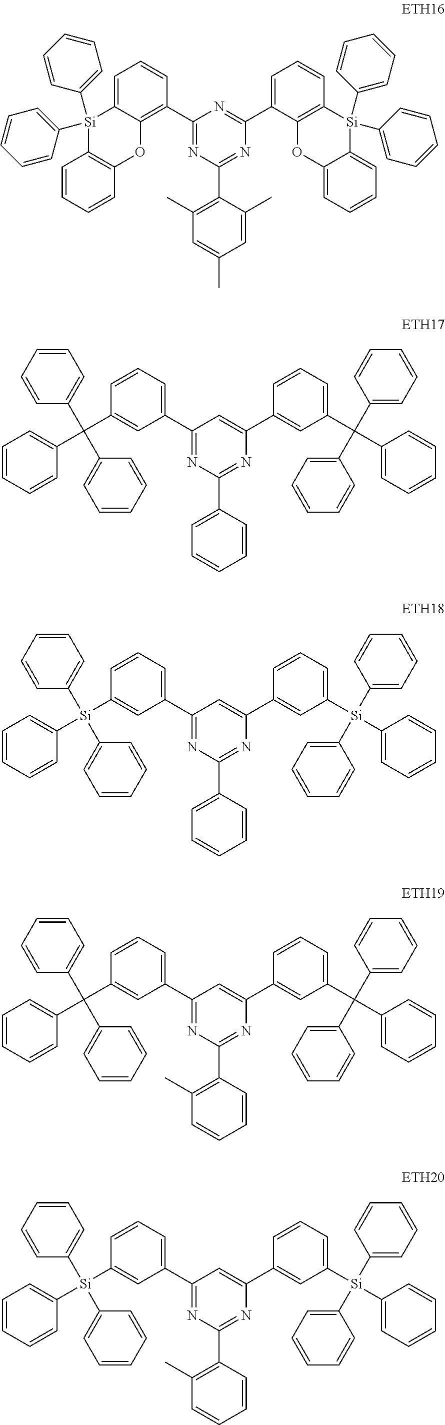

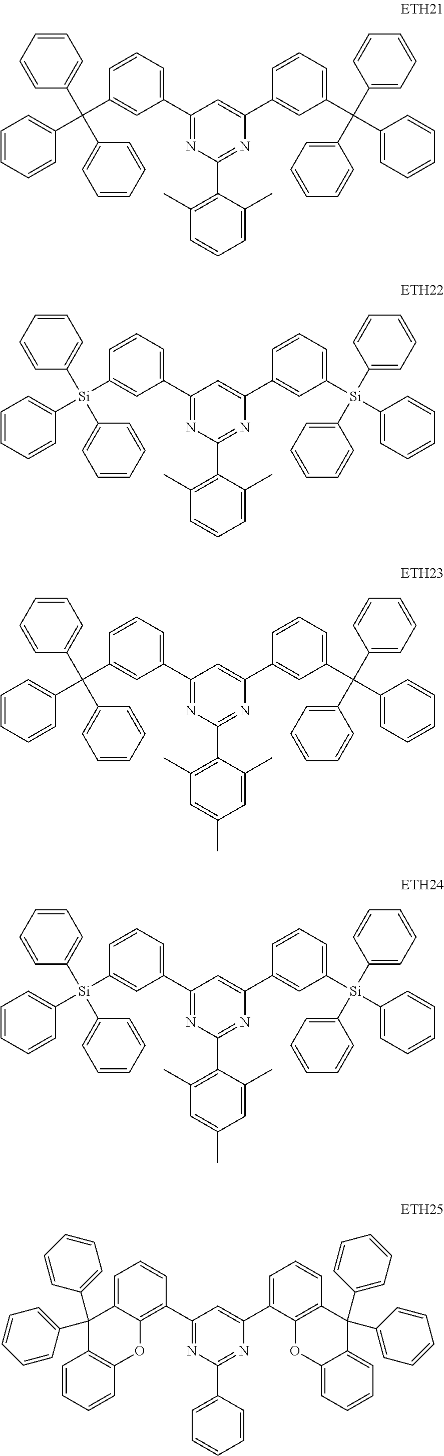

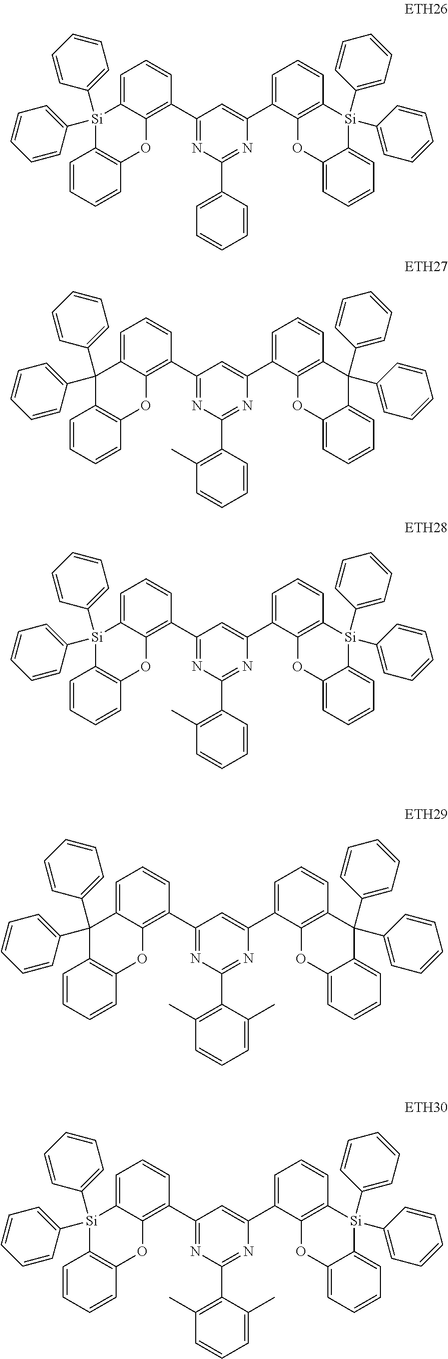

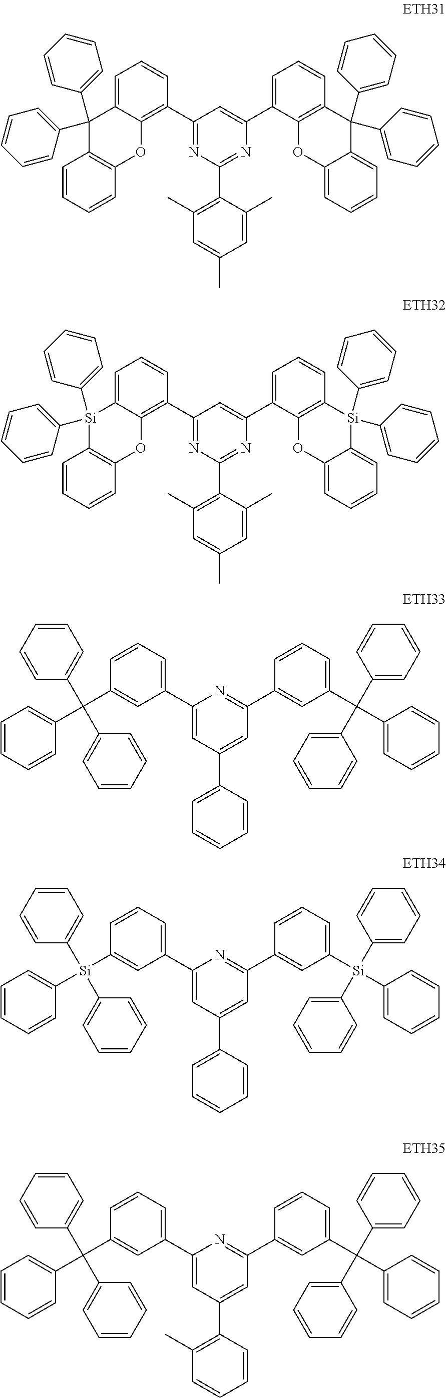

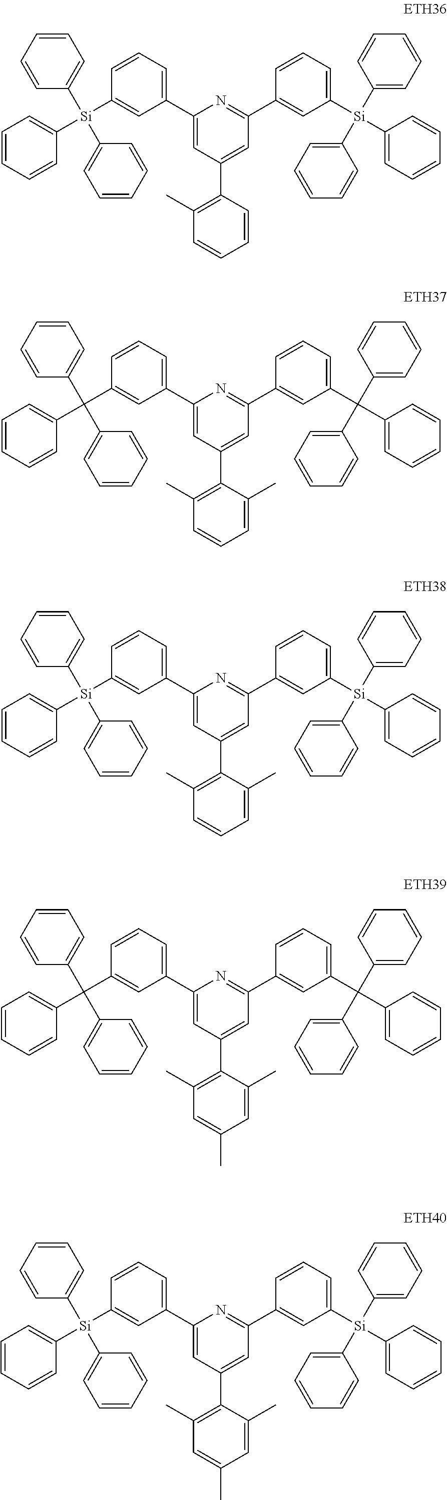

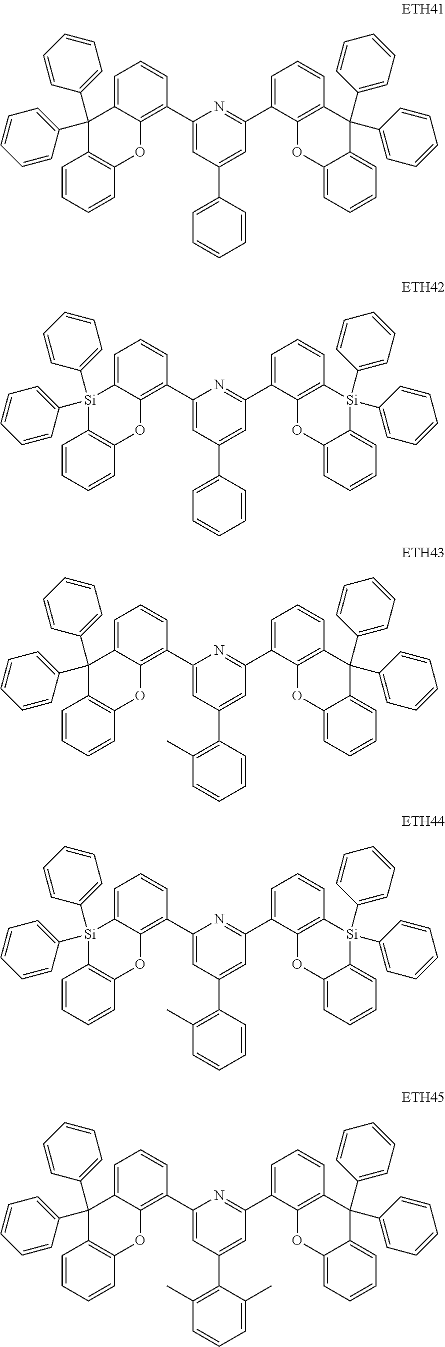

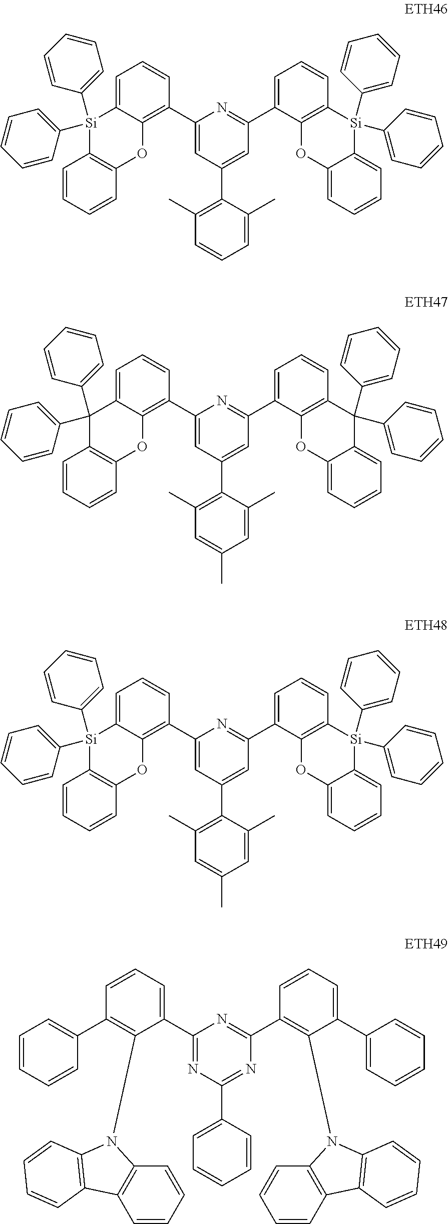 [SMILES: Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1 ; c1ccc(-c2nc(-c3cccc(-c4ccccc4)c3-n3c4ccccc4c4ccccc43)nc(-c3cccc(-c4ccccc4)c3-n3c4ccccc4c4ccccc43)n2)cc1]

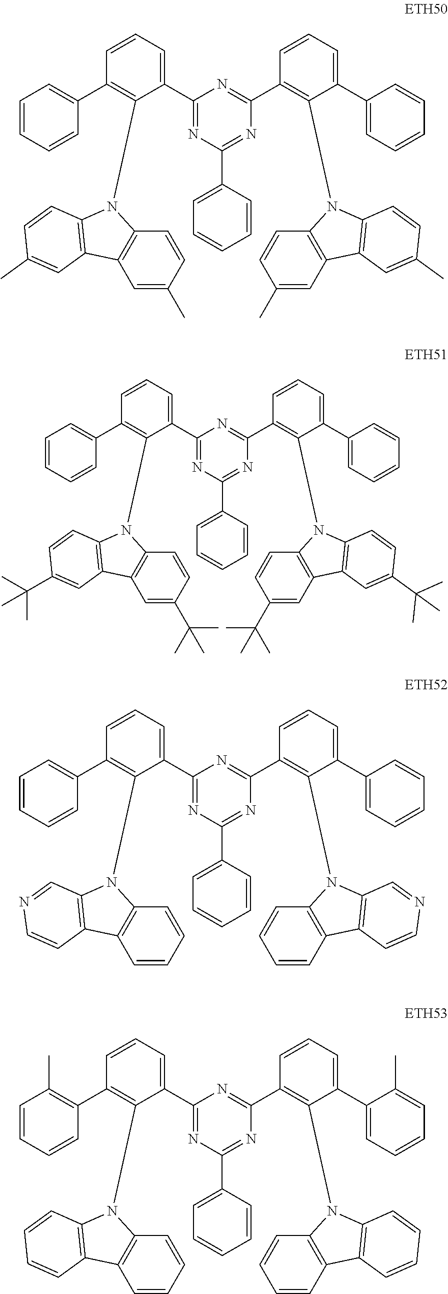

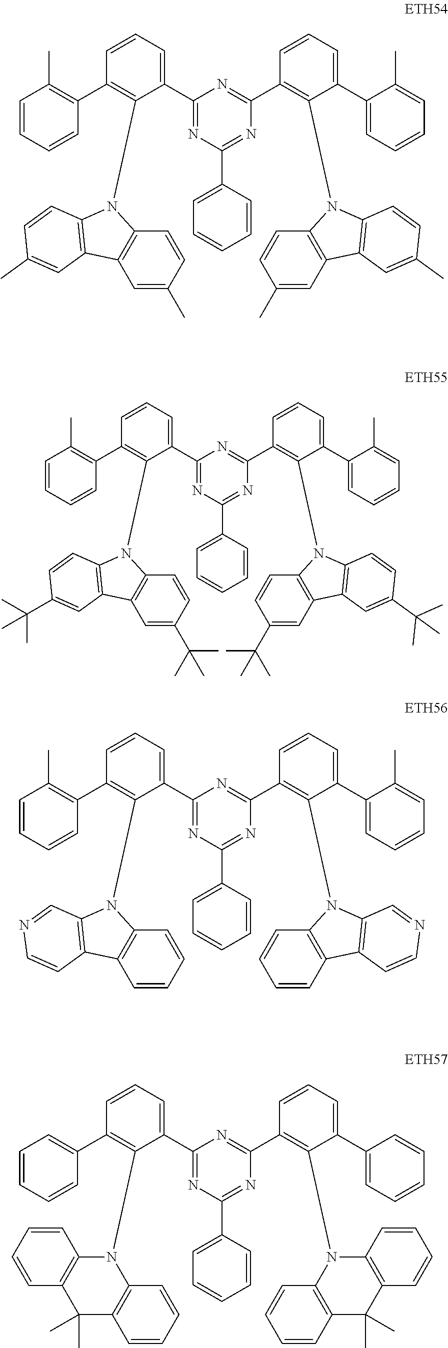

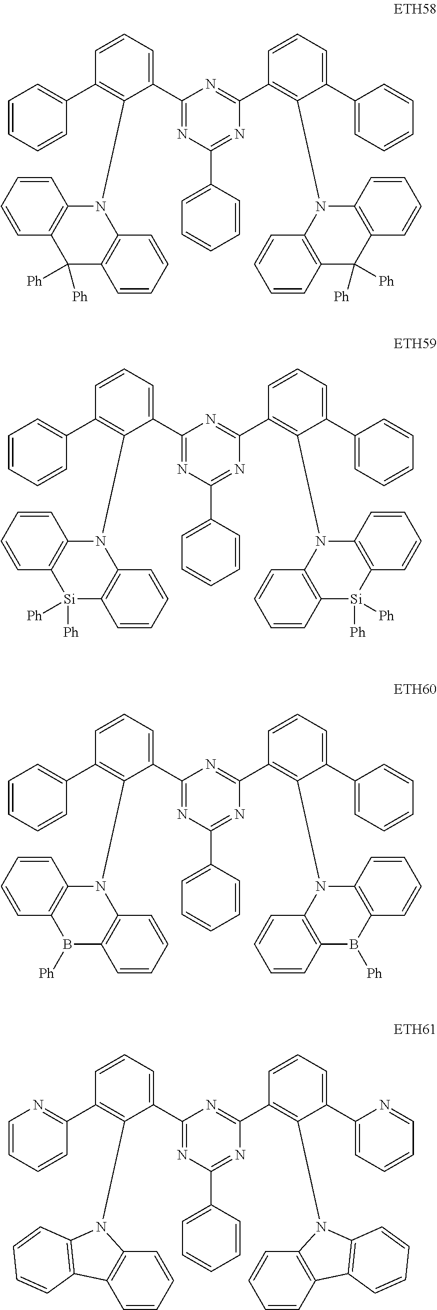

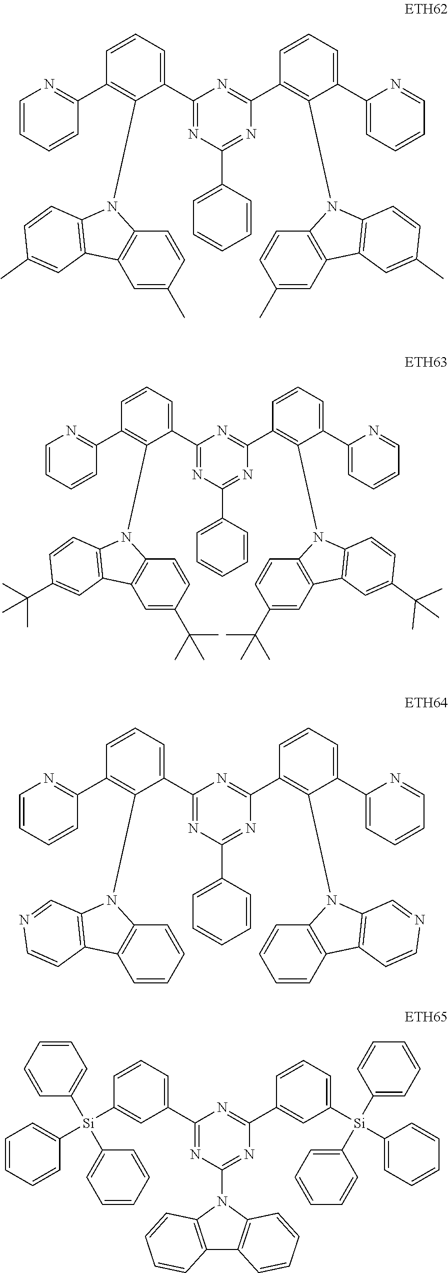

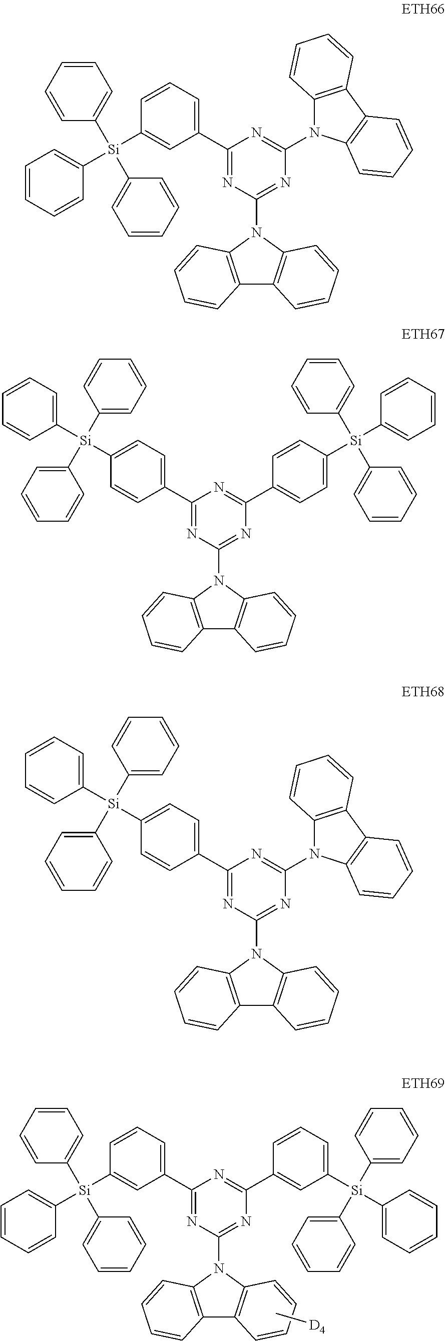

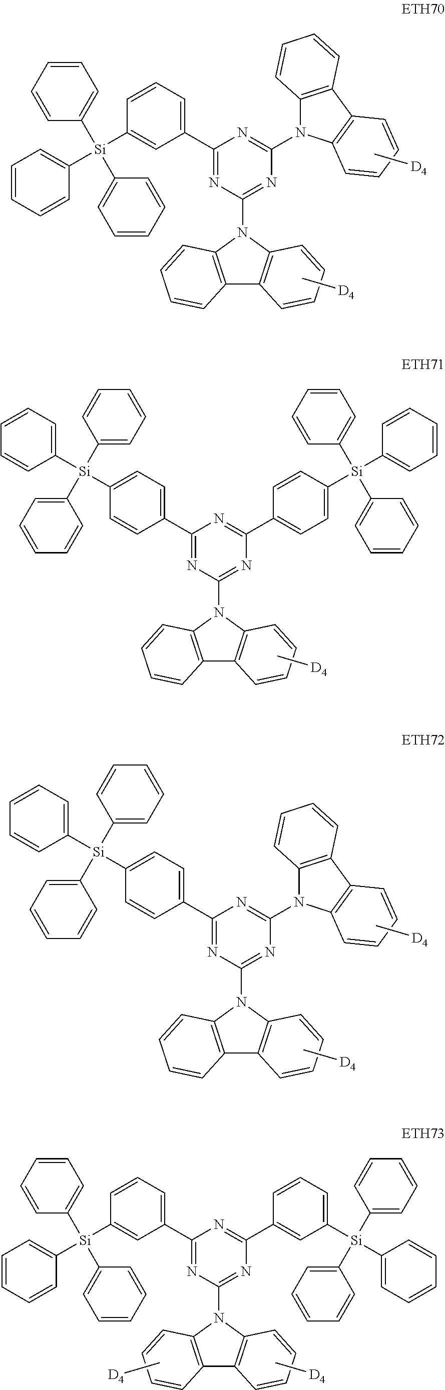

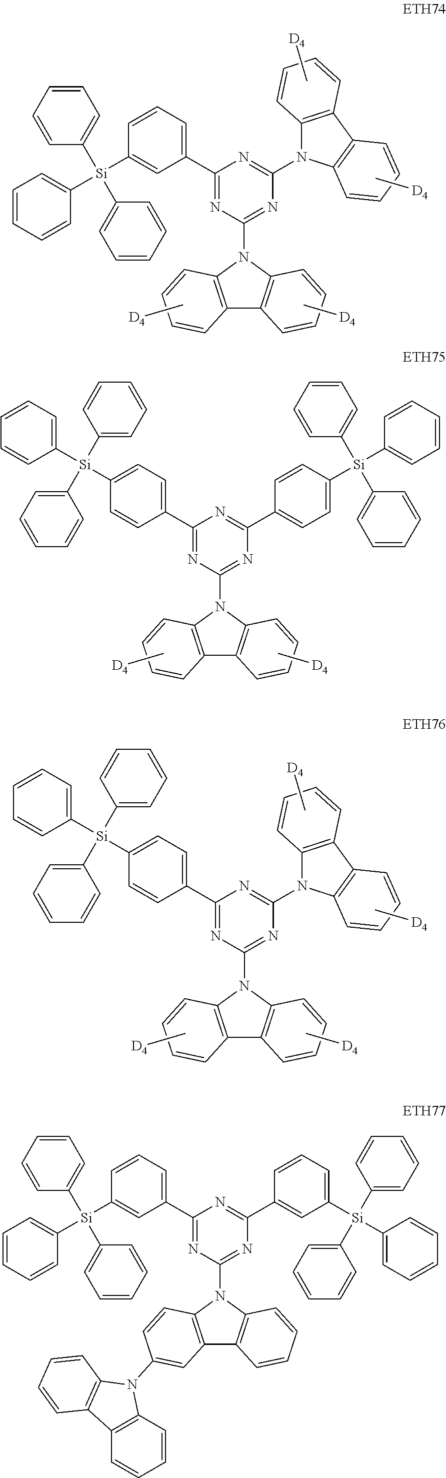

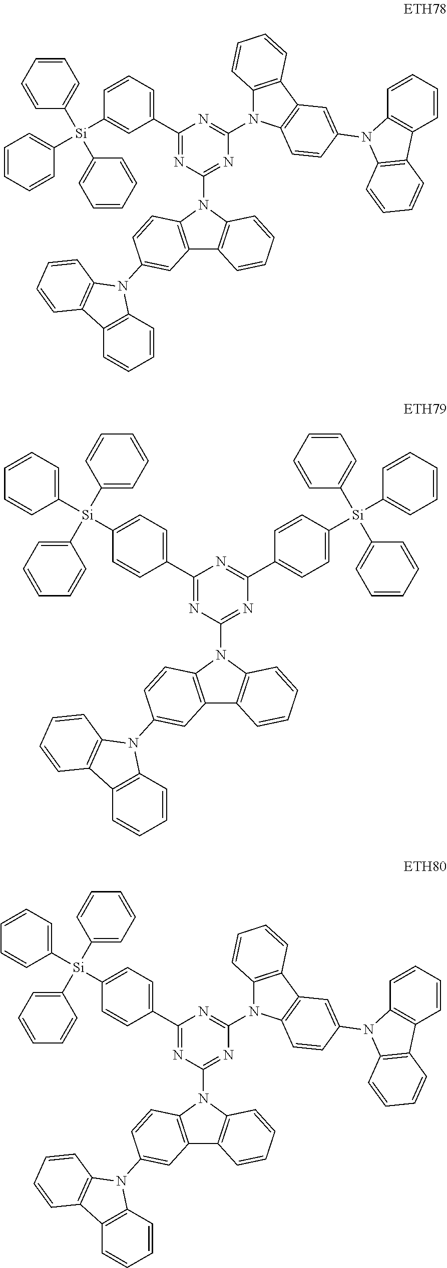 [SMILES: c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1 ; c1ccc([Si](c2ccccc2)(c2ccccc2)c2ccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)cc2)cc1]

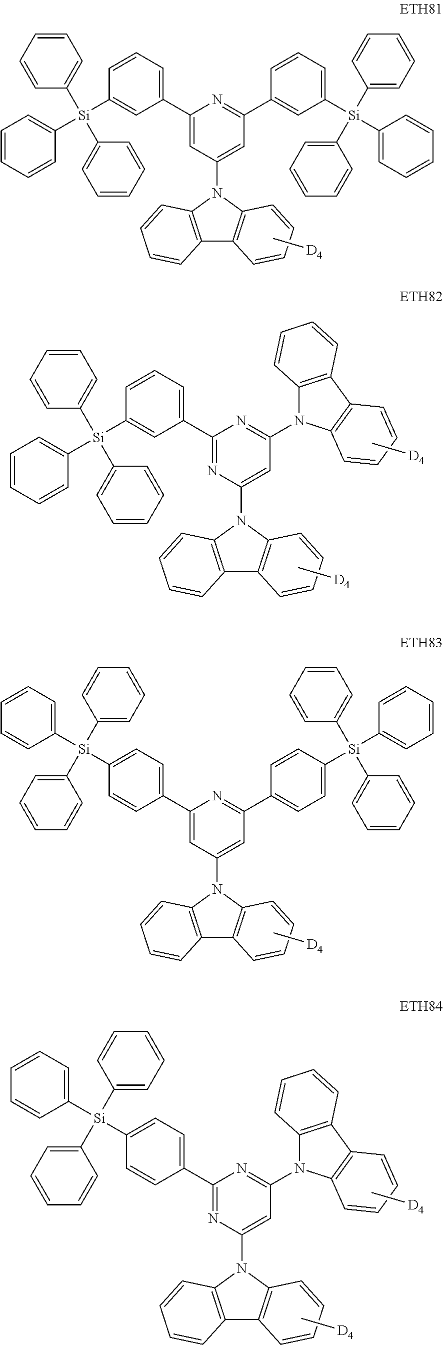

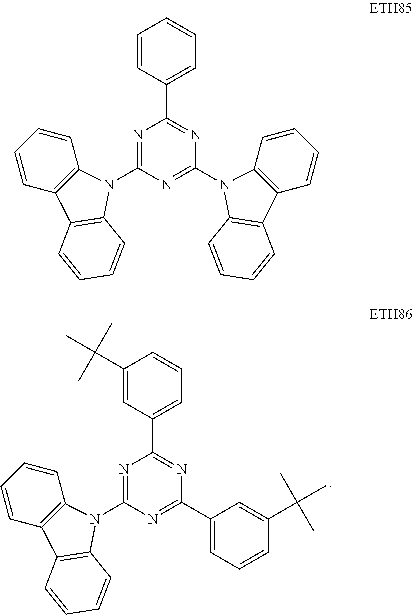 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1]

In one or more embodiments, the emission layer EML may include a fourth compound represented by Formula M-b below. The fourth compound represented by Formula M-b may include platinum (Pt) as a central metal. For example, the fourth compound may be used as a phosphorescence sensitizer of the emission layer EML.

In some embodiments, the fourth compound may be used as the phosphorescence dopant material of the emission layer EML:

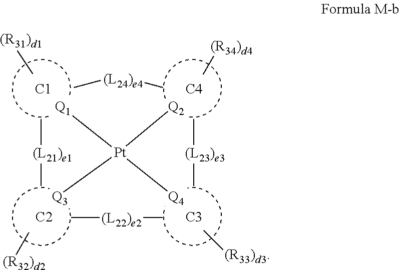

In Formula M-b, Q₁ to Q₄ may be each independently C or N, C1 to C4 may be each independently a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may be each independently a direct linkage,

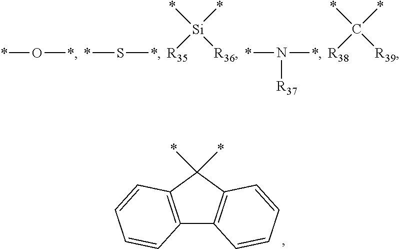

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may be each independently 0 or 1.

In Formula M-b, R₃₁ to R₃₉ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may be each independently an integer of 0 to 4.

The compound represented by Formula M-b may be used as a blue phosphorescence dopant or a green phosphorescence dopant. The compound represented by Formula M-b may be represented by any one of the compounds below. However, the compounds below are illustrations, and the compound represented by Formula M-b is not limited to the compounds represented below:

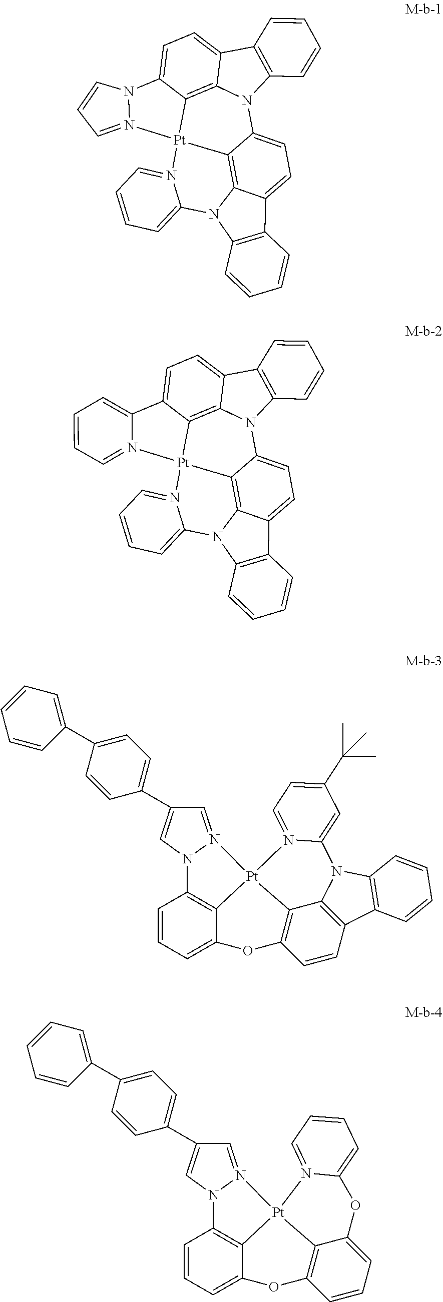

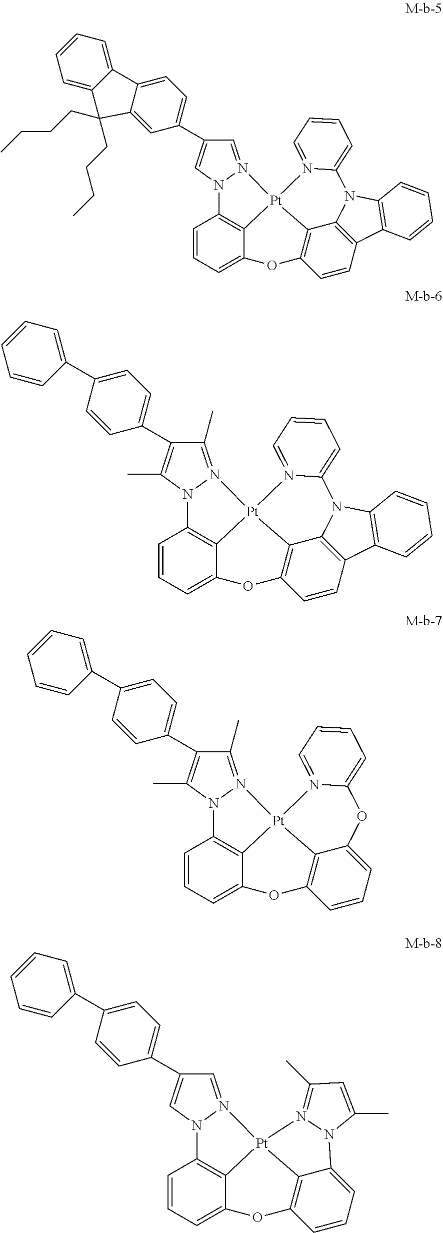

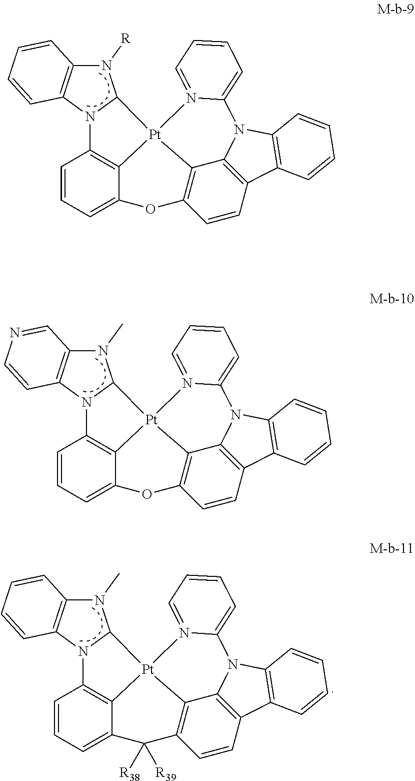

In the compounds above, R, R₃₈, and R₃₉ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include a compound represented by Formula M-a below. The compound represented by Formula M-a may be used as a phosphorescence dopant material:

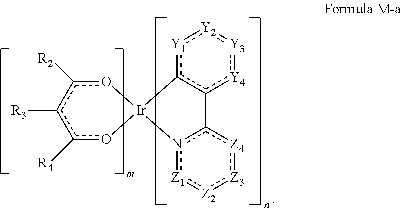

In Formula M-a, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In Formula M-a, “m” is 0 or 1, and “n” is 2 or 3. In Formula M-a, if “m” is 0, “n” is 3, and if “m” is 1, “n” is 2.

The compound represented by Formula M-a may be used as a phosphorescence dopant. The compound represented by Formula M-a may be represented by any one of Compounds M-a1 to M-a25 below. However, Compounds M-a1 to M-a25 are illustrations, and the compound represented by Formula M-a is not limited to those represented by Compounds M-a1 to M-a25:

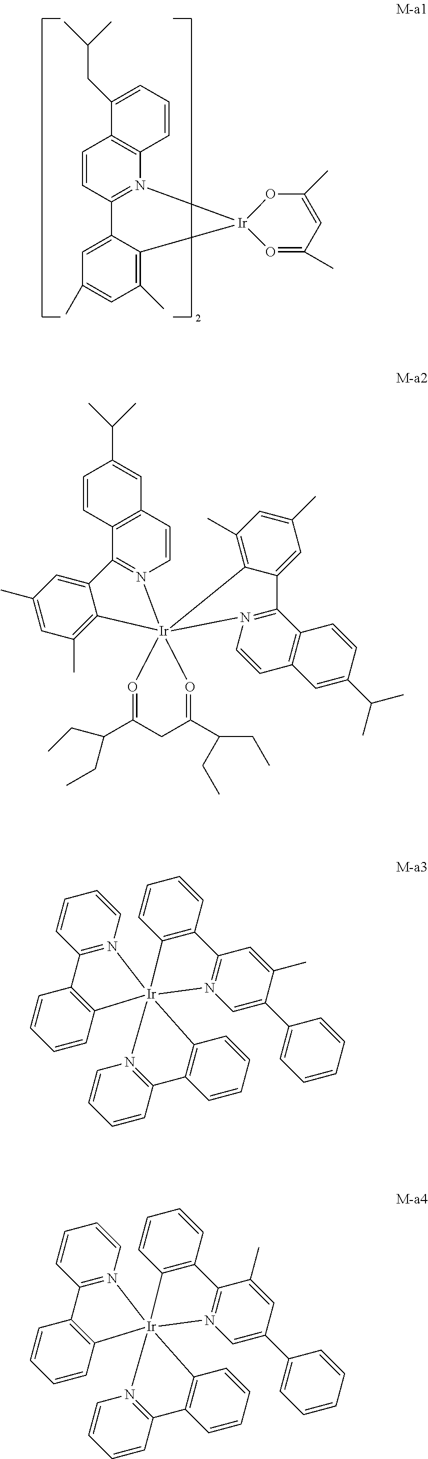

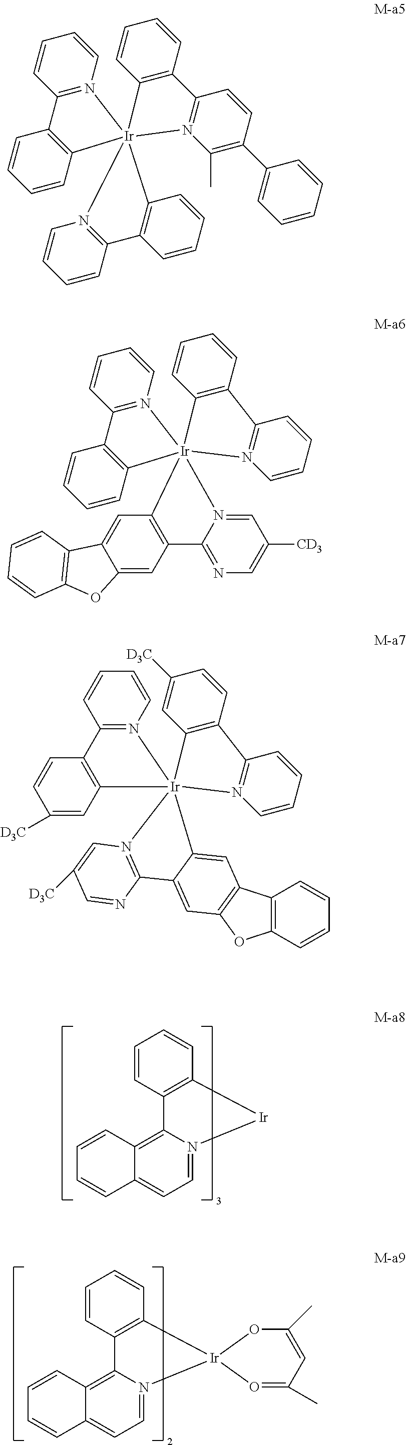

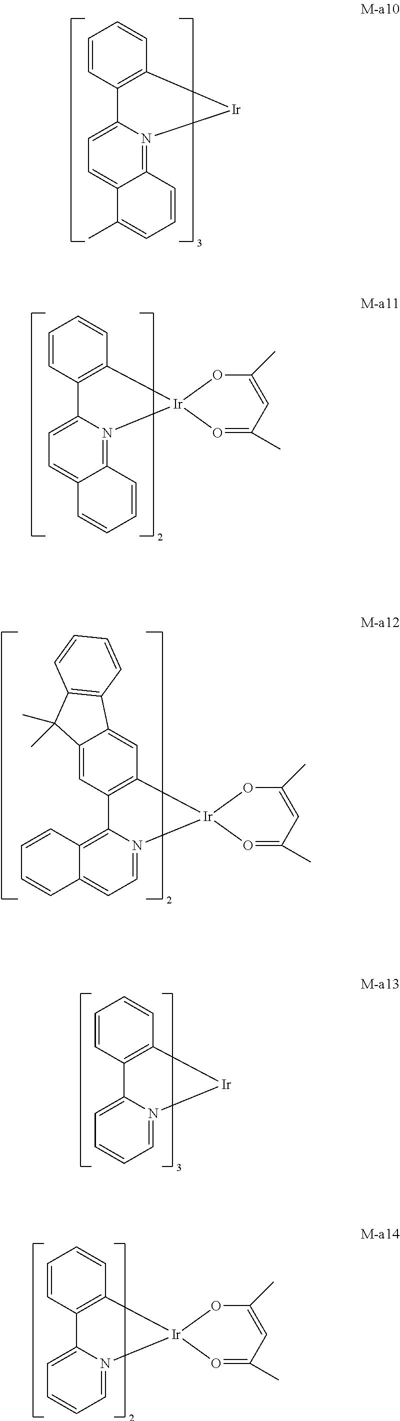

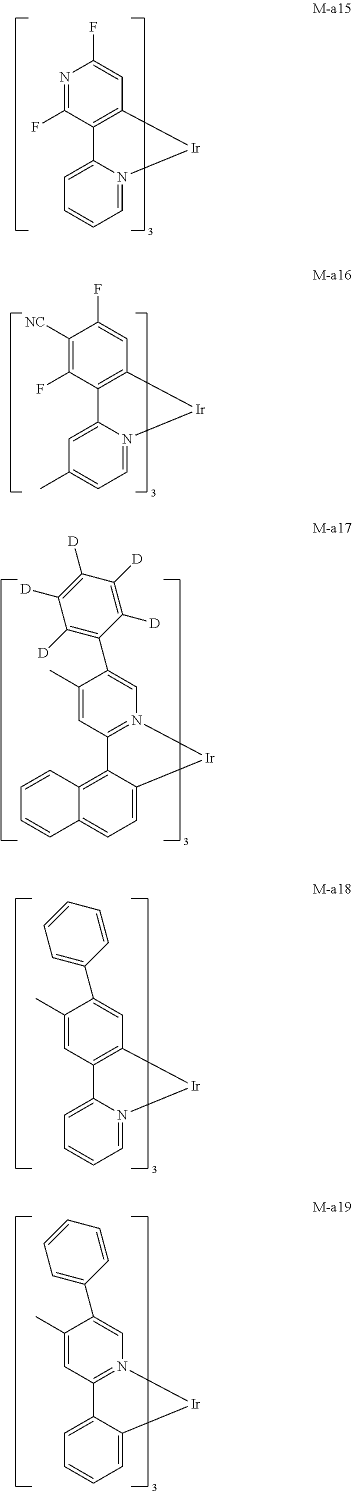

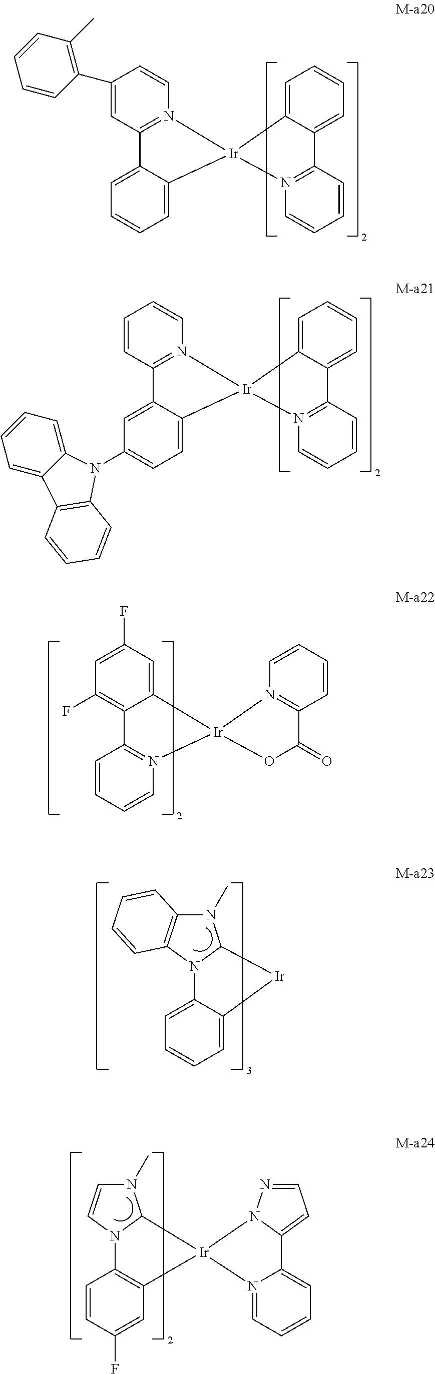

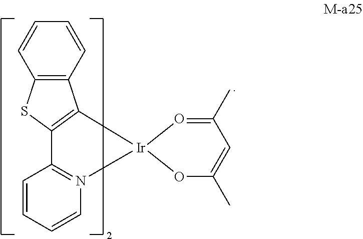

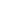

Compound M-a1 and Compound M-a2 may be used as red dopant materials, and Compound M-a3 to Compound M-a7 may be used as green dopant materials.

In one or more embodiments, the emission layer EML may include a 15 compound represented by Formula E-2a or Formula E-2b below. The compound represented by Formula E-2a or Formula E-2b below may be used as a phosphorescence host material:

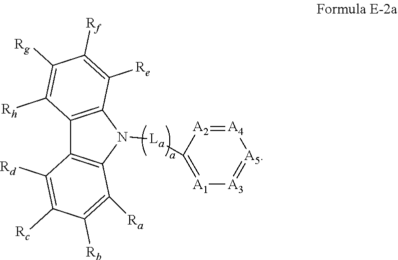

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if “a” is an integer of 2 or more, multiple La may be each independently a substituted or unsubstituted arylene group of 6 to 30 ring-forming carbon atoms, or a substituted or unsubstituted heteroarylene group of 2 to 30 ring-forming carbon atoms.

In one or more embodiments, in Formula E-2a, A₁ to A₅ may be each independently N or CRi. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Rₐ to Rᵢ may be combined with an adjacent group to form a hydrocarbon ring or a heterocycle including N, O, S, etc. as a ring-forming atom. In one or more embodiments, in Formula E-2a, two or three selected from A₁ to A₅ may be N, and the remainder may be CRᵢ.

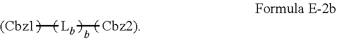

In Formula E-2b, Cbz1 and Cbz2 may be each independently an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, multiple L_{b} may be each independently a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one of the compounds in Compound Group E-2 below. However, the compounds listed in Compound Group E-2 below are only illustrations, and the compound represented by Formula E-2a or Formula E-2b is not limited to the compounds represented in Compound Group E-2 below.

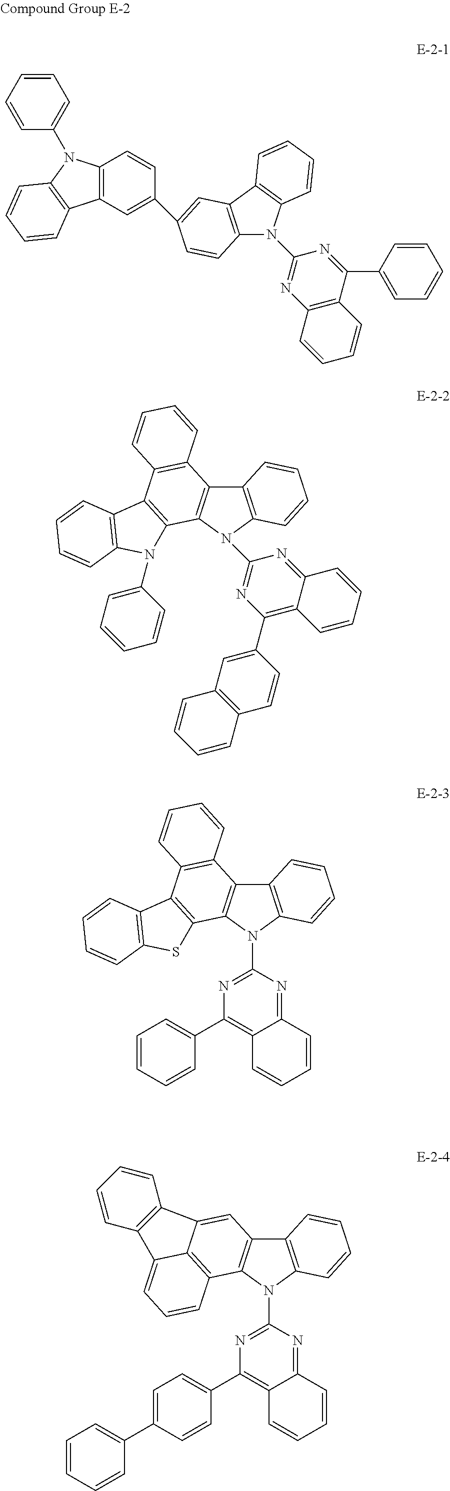 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1 ; c1ccc(-c2ccc(-c3nc(-n4c5ccccc5c5cc6c7c(cccc7c54)-c4ccccc4-6)nc4ccccc34)cc2)cc1]

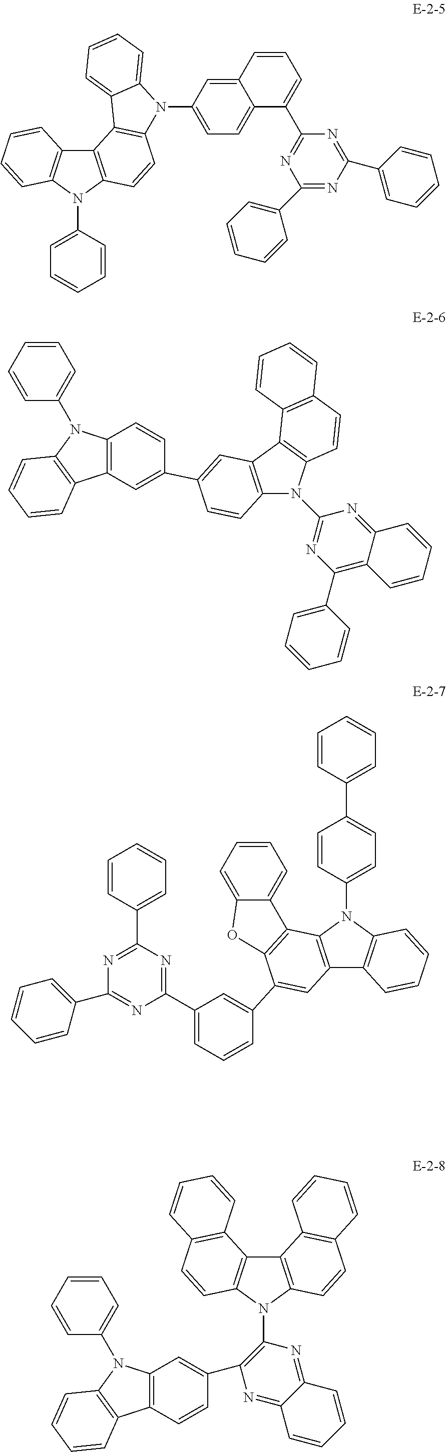 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

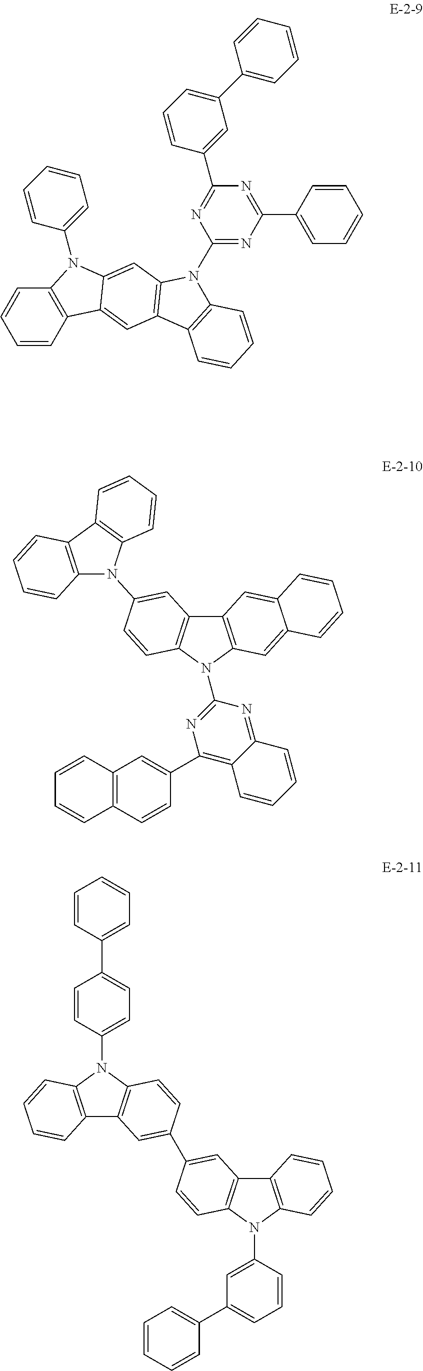 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

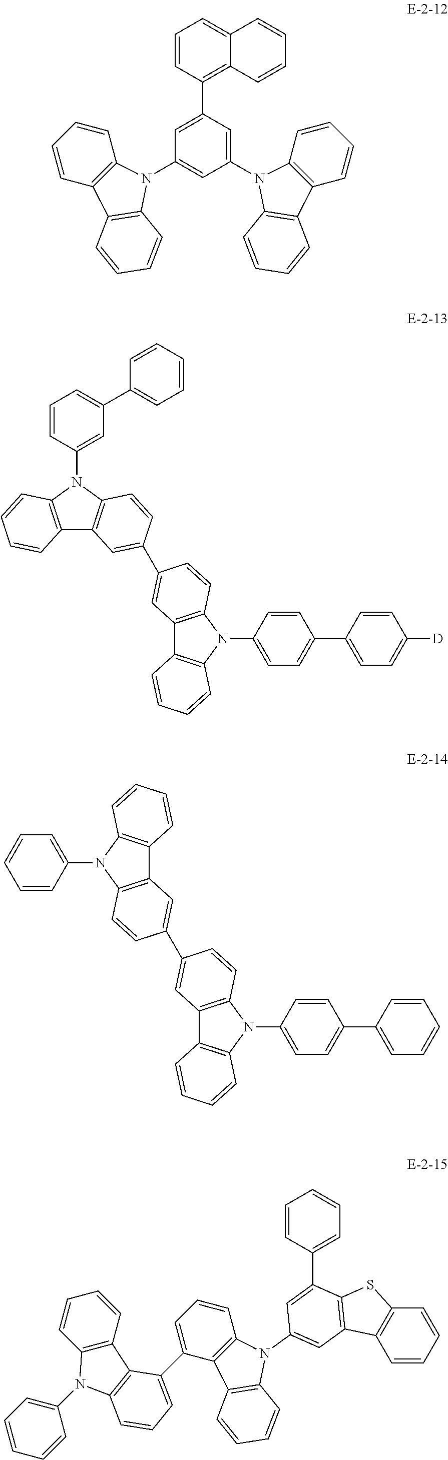 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

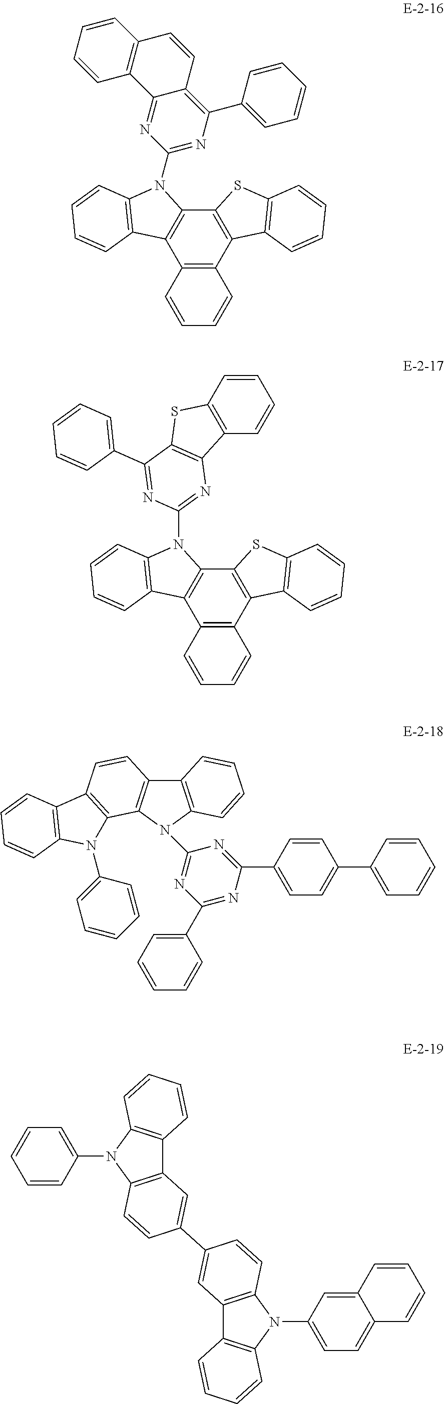 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

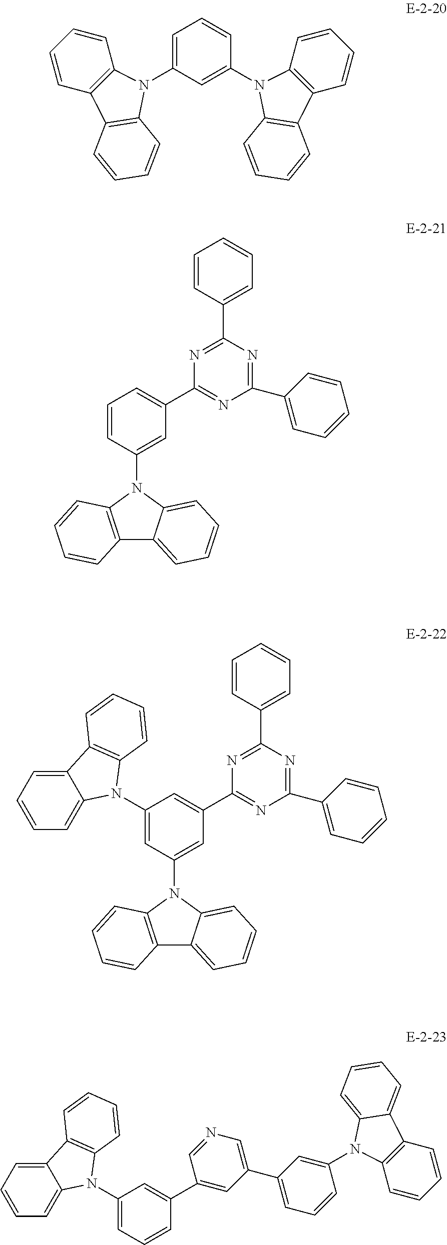 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

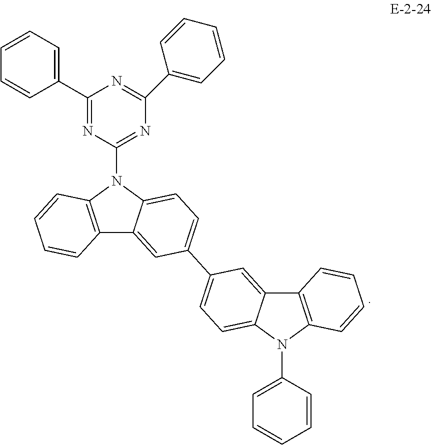 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material suitable in the art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and/or 1,3,5-tris(1-phenyl-1H-benzo[d]imidazole-2-yl)benzene (TPBi). However, one or more embodiments of the present disclosure is not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may further include a compound represented by any one of Formula F-a to Formula F-c below. The compounds represented by Formula F-a to Formula F-c may be used as fluorescence dopant materials.

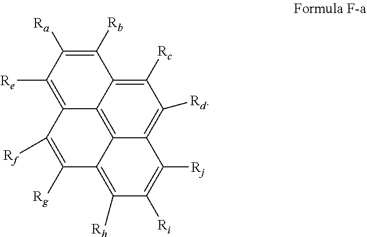

In Formula F-a, two selected from Rₐ to Rⱼ may be each independently substituted with *—NAr₁Ar₂ (e.g., two selected from Rₐ to Rⱼ may be each independently *—NAr₁Ar₂). The remainder not substituted with *—NAr₁Ar₂ of Rₐ to Rⱼ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₃Ar₂, Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. For example, at least one of Ar₁ and/or Ar₂ may be a heteroaryl group including O or S as a ring-forming atom.

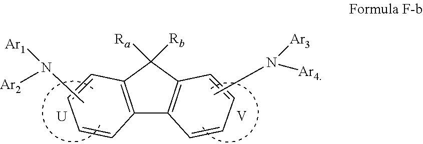

In Formula F-b, Rₐ and R_{b} may be each independently a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Ar₁ to Ar₄ may be each independently a substituted or unsubstituted aryl group of 6 to 30 ring-forming carbon atoms, or a substituted or unsubstituted heteroaryl group of 2 to 30 ring-forming carbon atoms.

In Formula F-b, U and V may be each independently a substituted or unsubstituted hydrocarbon ring of 5 to 30 ring-forming carbon atoms, or a substituted or unsubstituted heterocycle of 2 to 30 ring-forming carbon atoms. In Formula F-b, the number of rings represented by U and V may be each independently 0 or 1.

For example, in Formula F-b, if the number of U or V is 1, one ring forms a fused ring at the designated part by U or V, and if the number of U or V is 0, a ring is not present at the designated part by U or V. For example, if the number of U is 0, and the number of V is 1, or if the number of U is 1, and the number of V is 0, a fused ring having the fluorene core of Formula F-b may be a ring compound with four rings. If the number of both U and V is 0, the fused ring having a fluorene core of Formula F-b may be a ring compound with three rings. If the number of both U and V is 1, a fused ring having the fluorene core of Formula F-b may be a ring compound with five rings.

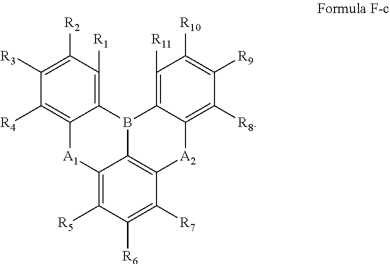

In Formula F-c, A1 and A2 may be each independently O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may be each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In Formula F-c, A₁ and A₂ may be each independently combined with the substituents of an adjacent ring to form a fused ring. For example, if A₁ and A₂ are each independently NRₘ, A₁ may be combined with R₄ or R₅ to form a ring. In addition, A₂ may be combined with R₇ or R₈ to form a ring.

In one or more embodiments, the emission layer EML may include as a dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and/or 4,4′-bis[2-(4-(N,N-diphenylamino)phenyl)vinyl]biphenyl (DPAVBi)), perylene and/or the derivatives thereof (for example, 2,5,8,11-tetra-t-butylperylene (TBP)), pyrene and/or the derivatives thereof (for example, 1,1-dipyrene, 1,4-dipyrenylbenzene, and/or 1,4-bis(N,N-diphenylamino)pyrene), etc.

The emission layer EML may include a suitable phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb), and/or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III)(FIr6), and/or platinum octaethyl porphyrin (PtOEP) may be used as the phosphorescence dopant. However, one or more embodiments of the present disclosure is not limited thereto.

The emission layer EML may include a quantum dot material. The core of the quantum dot may be selected from II-VI group compounds, III-VI group compounds, I-III-VI group compounds, III-V group compounds, III-II-V group compounds, IV-VI group compounds, IV group elements, IV group compounds, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and mixtures thereof.

The III-V group compound may include a binary compound such as In₂S₃, and/or In₂Se₃; a ternary compound such as InGaS₃ and/or InGaSe₃; and/or optional combinations thereof.

The I-III-VI group compound may be selected from the group consisting of a ternary compound selected from the group consisting of AgInS, AgInS₂, CuInS, CulnS₂, AgGaS₂, CuGaS₂, CuGaO₂, AgGaO₂, AgAlO₂ and mixtures thereof; and a quaternary compound such as AgInGaS₂ and/or CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof; a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof; and a quaternary compound selected from the group consisting of GaAlNP, GaAlNAs, GaAlNSb, GaAlPAs, GaAlPSb, GaInNP, GaInNAs, GalnNSb, GaInPAs, GaInPSb, InAlNP, InAlNAs, InAlNSb, InAlPAs, InAlPSb, and mixtures thereof. In one or more embodiments, the III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof; a ternary compound selected from the group consisting of SnSeS, SnSeTe, SnSTe, PbSeS, PbSeTe, PbSTe, SnPbS, SnPbSe, SnPbTe, and mixtures thereof; and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

In this case, the binary compound, the ternary compound, and/or the quaternary compound may be present at substantially uniform concentration in a particle or may be present at a partially different concentration distribution state in the same particle. In some embodiments, a core/shell structure in which one quantum dot wraps another quantum dot may be possible. The interface of the core and the shell may have a concentration gradient in which the concentration of an element present in the shell is decreased toward the center.

In some embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal oxide, a non-metal oxide, a semiconductor compound, and combinations thereof.

For example, the metal oxide and the non-metal oxide may each independently include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and/or NiO, and/or a ternary compound such as MgAl₂O₄, CoFe₂O₄, NiFe₂O₄ and/or CoMn₂O₄, but one or more embodiments of the present disclosure is not limited thereto.

The semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but one or more embodiments of the present disclosure is not limited thereto.

The quantum dot may have a full width of half maximum (FWHM) of emission wavelength spectrum of about 45 nm or less, for example, about 40 nm or less, or about 30 nm or less. Within any of these ranges, color purity and/or color reproducibility may be improved. In addition, light emitted via such quantum dot is emitted in all directions, and light view angle properties may be improved.

The shape of the quantum dot may be any suitable shape in the art, without specific limitation. For example, the shape of spherical, pyramidal, multi-arm, and/or cubic nanoparticle, nanotube, nanowire, nanofiber, nanoplate particle, etc. may be used.

The quantum dot may control the color of light emitted according to the particle size, and accordingly, the quantum dot may have various emission colors such as blue, red and green.

In the light emitting elements ED of embodiments, as shown in FIG. 3 to FIG. 7, the electron transport region ETR is provided on the emission layer EML. The electron transport region ETR may include at least one of a hole blocking layer HBL, an electron transport layer ETL and/or an electron injection layer EIL. However, one or more embodiments of the present disclosure is not limited thereto.

The electron transport region ETR may have a single layer formed using a single material, a single layer formed using multiple different materials, or a multilayer structure having multiple layers formed using multiple different materials. For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using multiple different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include the above-described third compound represented by Formula ET-1 above. The electron transport region ETR may include an anthracene-based compound. However, one or more embodiments of the present disclosure is not limited thereto. The electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and/or mixtures thereof, without limitation.

In one or more embodiments, the electron transport region ETR may include a metal halide (such as LiF, NaCl, CsF, RbCl, RbI, CuI and/or KI), a metal in lanthanoides such as Yb, and/or a co-depositing material of the metal halide and the metal in lanthanoides. For example, the electron transport region ETR may include KI:Yb, RbI:Yb, LiF:Yb, etc., as the co-depositing material. In one or more embodiments, the electron transport region ETR may use a metal oxide such as Li₂O and/or BaO, and/or 8-hydroxy-lithium quinolate (Liq). However, one or more embodiments of the present disclosure is not limited thereto. The electron transport region ETR also may be formed using a mixture material of an electron transport material and an insulating organo metal salt. The organo metal salt may be a material having an energy band gap of about 4 eV or more. For example, the organo metal salt may include, for example, one or more of metal acetates, metal benzoates, metal acetoacetates, metal acetylacetonates, and/or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) and/or 4,7-diphenyl-1,10-phenanthroline (Bphen), in addition to the aforementioned materials. However, one or more embodiments of the present disclosure is not limited thereto.

The electron transport region ETR may include the compounds of the electron transport region in at least one of an electron injection layer EIL, an electron transport layer ETL, and/or a hole blocking layer HBL.

If the electron transport region ETR includes the electron transport layer ETL, the thickness of the electron transport layer ETL may be from about 100 Å to about 1,000 Å, for example, from about 150 Å to about 500 Å. If the thickness of the electron transport layer ETL satisfies any of the above-described ranges, satisfactory or suitable electron transport properties may be obtained without substantial increase of a driving voltage. If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies any of the above described ranges, satisfactory or suitable electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but one or more embodiments of the present disclosure is not limited thereto. For example, if the first electrode EL1 is an anode, the second cathode EL2 may be a cathode, and if the first electrode EL1 is a cathode, the second electrode EL2 may be an anode. The second electrode EL2 may include at least one of Ag, Mg, Cu, Al, Pt, Pd, Au, Ni, Nd, Ir, Cr, Li, Ca, LiF, Mo, Ti, W, In, Sn, Zn, a compound of two or more thereof, a mixture of two or more thereof, and/or an oxide thereof.

The second electrode EL2 may be a transmissive electrode, a transflective electrode or a reflective electrode. If the second electrode EL2 is the transmissive electrode, the second electrode EL2 may include a transparent metal oxide, for example, ITO, IZO, ZnO, ITZO, etc.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds including thereof, and/or mixtures thereof (for example, AgMg, AgYb, or MgYb). In some embodiments, the second electrode EL2 may have a multilayered structure including a reflective layer or a transflective layer formed using any of the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, and/or oxides of the aforementioned metal materials.

In one or more embodiments, the second electrode EL2 may be connected (e.g., electrically coupled) with an auxiliary electrode. If the second electrode EL2 is connected with the auxiliary electrode, the resistance of the second electrode EL2 may decrease.

In one or more embodiments, on the second electrode EL2 in the light emitting element ED of one or more embodiments, a capping layer CPL may be further provided. The capping layer CPL may include a multilayer or a single layer.

In one or more embodiments, the capping layer CPL may be an organic layer or an inorganic layer. For example, if the capping layer CPL includes an inorganic material, the inorganic material may include an alkali metal compound such as LiF, an alkaline earth metal compound such as SiON, SiNx, SiOy, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc. The capping layer CPL may include an epoxy resin, and/or acrylate such as methacrylate. In some embodiments, the capping layer CPL may include at least one of Compounds P1 to P5 below, but one or more embodiments of the present disclosure is not limited thereto.

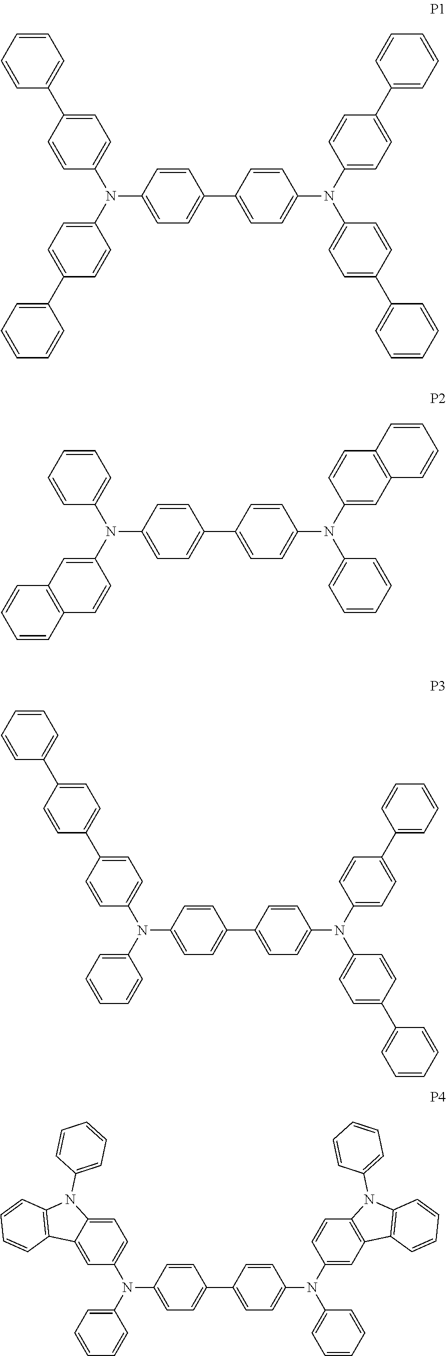 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

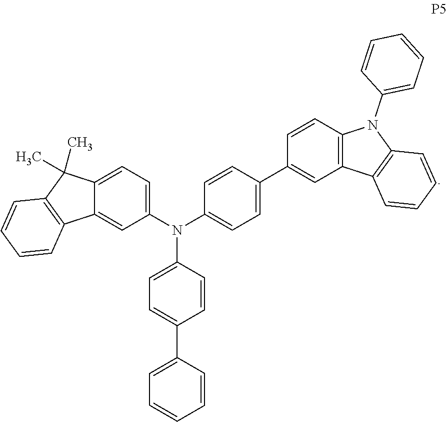 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, the refractive index of the capping layer CPL with respect to light in a wavelength range of about 550 nm to about 660 nm may be about 1.6 or more.

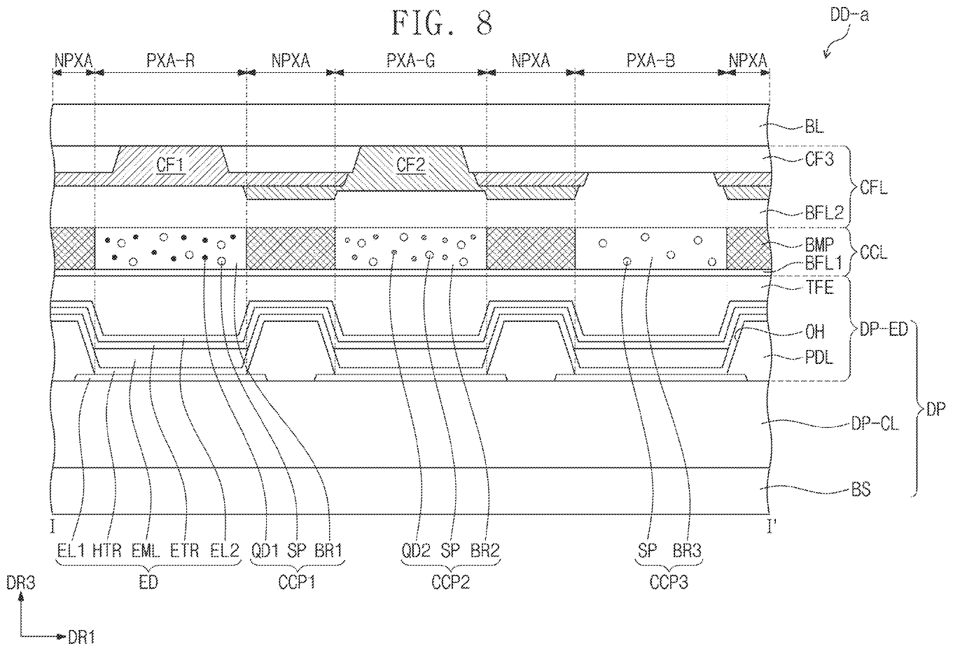

FIG. 8 to FIG. 11 are cross-sectional views of display devices according to embodiments of the present disclosure. Hereinafter, in the explanation of the display devices of embodiments, referring to FIG. 8 to FIG. 11, the overlapping parts with the explanation provided in connection with FIG. 1 to FIG. 7 will not be explained again, and the different features will be explained chiefly.

Referring to FIG. 8, a display device DD-a according to one or more embodiments may include a display panel DP including a display element layer DP-ED, a light controlling layer CCL provided on the display panel DP, and a color filter layer CFL. In one or more embodiments shown in FIG. 8, the display panel DP includes a base layer BS, a circuit layer DP-CL provided on the base layer BS and a display element layer DP-ED, and the display element layer DP-ED may include a light emitting element ED.

The light emitting element ED may include a first electrode EL1, a hole transport region HTR provided on the first electrode EL1, an emission layer EML provided on the hole transport region HTR, an electron transport region ETR provided on the emission layer EML, and a second electrode EL2 provided on the electron transport region ETR. The structures of the light emitting elements of FIG. 3 to FIG. 7 may be applied to the structure of the light emitting element ED shown in FIG. 8. In one or more embodiments, the light emitting element ED may include the amine compound of one or more embodiments.

Referring to FIG. 8, the emission layer EML may be provided in an opening portion OH defined in a pixel definition layer PDL. For example, the emission layer EML divided by the pixel definition layer PDL and correspondingly provided to each of luminous areas PXA-R, PXA-G and PXA-B may emit light in the same wavelength region. In the display device DD-a of one or more embodiments, the emission layer EML may emit blue light. In one or more embodiments, the emission layer EML may be provided as a common layer for all luminous areas PXA-R, PXA-G and PXA-B.

The light controlling layer CCL may be provided on the display panel DP. The light controlling layer CCL may include a light converter. The light converter may be a quantum dot and/or a phosphor. The light converter may transform the wavelength of light provided and then emit the transformed light. For example, the light controlling layer CCL may be a layer including a quantum dot and/or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 8, a partition pattern BMP may be provided between the separated light controlling parts CCP1, CCP2 and CCP3, but one or more embodiments of the present disclosure is not limited thereto. In FIG. 8, the partition pattern BMP is shown not to be overlapped with the light controlling parts CCP1, CCP2 and CCP3, but in some embodiments, at least a portion of the edge of the light controlling parts CCP1, CCP2 and CCP3 may be overlapped with the partition pattern BMP.

The light controlling layer CCL may include a first light controlling part CCP1 including a first quantum dot QD1 converting (e.g., configured to convert) first color light provided from the light emitting element ED into second color light, a second light controlling part CCP2 including a second quantum dot QD2 converting (e.g., configured to convert) first color light into third color light, and a third light controlling part CCP3 transmitting (e.g., configured to transmit) first color light. In one or more embodiments, the first light controlling part CCP1 may provide red light which is the second color light, and the second light controlling part CCP2 may provide green light which is the third color light. The third color controlling part CCP3 may transmit and provide blue light which is the first color light provided from the light emitting element ED. For example, the first quantum dot QD1 may be a red quantum dot, and the second quantum dot QD2 may be a green quantum dot. For the quantum dots QD1 and QD2, the same descriptions as those described above may be applied.

In one or more embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot (may exclude or not include any quantum dot) but may include the scatterer SP.

The scatterer SP may be an inorganic particle. For example, the scatterer SP may include at least one of TiO₂, ZnO, Al₂O₃, SiO₂, and/or hollow silica. The scatterer SP may include at least one of TiO₂, ZnO, Al₂O₃, SiO₂, and/or hollow silica, or may be a mixture of two or more of TiO₂, ZnO, Al₂O₃, SiO₂, and/or hollow silica.

Each of the first light controlling part CCP1, the second light controlling part CCP2, and the third light controlling part CCP3 may include base resins BR1, BR2 and BR3, respectively, for dispersing the quantum dots QD1 and QD2 and the scatterer SP, respectively. In one or more embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3.

The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may each independently be acrylic resins, urethane-based resins, silicone-based resins, epoxy-based resins, etc. The base resins BR1, BR2 and BR3 may be transparent resins. In one or more embodiments, the first base resin BR1, the second base resin BR2 and the third base resin BR3 may be the same or different from each other.

The light controlling layer CCL may include a barrier layer BFL1. The barrier layer BFL1 may play the role of blocking or reducing the penetration of moisture and/or oxygen (hereinafter, will be referred to as “humidity/oxygen”). The barrier layer BFL1 may block or reduce the exposure of the light controlling parts CCP1, CCP2 and CCP3 to humidity/oxygen. In one or more embodiments, the barrier layer BFL1 may cover the light controlling parts CCP1, CCP2 and CCP3. In some embodiments, the barrier layer BFL2 may be provided between a color filter layer CFL and the light controlling parts CCP1, CCP2 and CCP3.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, and/or a metal thin film securing light transmittance. In one or more embodiments, the barrier layers BFL1 and BFL2 may further include an organic layer. The barrier layers BFL1 and BFL2 may be composed of a single layer or multiple layers.

In the display device DD of one or more embodiments, the color filter layer CFL may be provided on the light controlling layer CCL. For example, the color filter layer CFL may be provided directly on the light controlling layer CCL. In this case, the barrier layer BFL2 may not be provided (e.g., may be omitted).

The color filter layer CFL may include filters CF1, CF2 and CF3. The color filter layer CFL may include a first filter CF1 transmitting (e.g., configured to transmit) second color light, a second filter CF2 transmitting (e.g., configured to transmit) third color light, and a third filter CF3 transmitting (e.g., configured to transmit) first color light. For example, the first filter CF1 may be a red filter, the second filter CF2 may be a green filter, and the third filter CF3 may be a blue filter. The first to third filters CF1, CF2 and CF3 may be provided corresponding to a red luminous area PXA-R, a green luminous area PXA-G and a blue luminous area PXA-B, respectively.

Each of the filters CF1, CF2 and CF3 may include a polymer photosensitive resin and a pigment and/or dye. The first filter CF1 may include a red pigment and/or dye, the second filter CF2 may include a green pigment and/or dye, and the third filter CF3 may include a blue pigment and/or dye. However, one or more embodiments of the present disclosure is not limited thereto, and the third filter CF3 may not include the pigment or dye. The third filter CF3 may include a polymer photosensitive resin and not include a pigment or dye. The third filter CF3 may be transparent. The third filter CF3 may be formed using a transparent photosensitive resin.

In one or more embodiments, the first filter CF1 and the second filter CF2 may be yellow filters. The first filter CF1 and the second filter CF2 may be provided in one body without distinction (e.g., may be integral with each other).

In some embodiments, the color filter layer CFL may further include a light blocking part. The light blocking part may be a black matrix. The light blocking part may be formed by including an organic light blocking material and/or an inorganic light blocking material, including a black pigment and/or black dye. The light blocking part may prevent or reduce light leakage and may divide the boundaries among adjacent filters CF1, CF2 and CF3.

On the color filter layer CFL, a base substrate BL may be provided. The base substrate BL may be a member providing a base surface on which the color filter layer CFL, the light controlling layer CCL, etc. are provided. The base substrate BL may be a glass substrate, a metal substrate, a plastic substrate, etc. However, one or more embodiments of the present disclosure is not limited thereto, and the base substrate BL may be an inorganic layer, an organic layer, or a composite material layer. In some embodiments, the base substrate BL may not be provided (e.g., may be omitted).

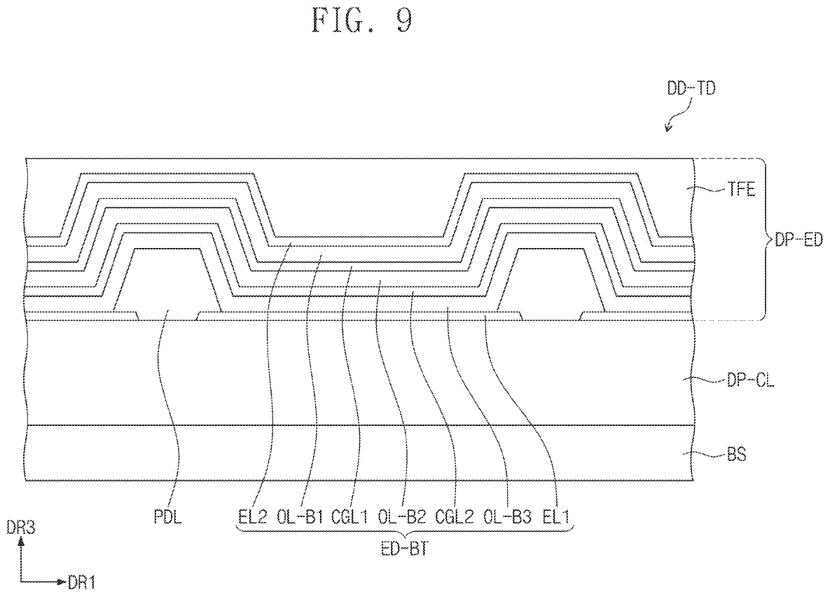

FIG. 9 is a cross-sectional view showing a portion of the display device according to one or more embodiments. In FIG. 9, the cross-sectional view of a portion corresponding to the display panel DP in FIG. 8 is shown. In a display device DD-TD of one or more embodiments, the light emitting element ED-BT may include multiple light emitting structures OL-B1, OL-B2 and OL-B3. The light emitting element ED-BT may include oppositely provided first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 8), a hole transport region HTR and an electron transport region ETR provided with the emission layer EML (FIG. 8) therebetween. For example, the light emitting element ED-BT included in the display device DD-TD of one or more embodiments may be a light emitting element of a tandem structure including multiple emission layers. At least one of multiple light emitting structures OL-B1, OL-B2 and/or OL-B3 may include the amine compound of one or more embodiments.

In one or more embodiments shown in FIG. 9, light emitted from the light emitting structures OL-B1, OL-B2 and OL-B3 may be all blue light. However, one or more embodiments of the present disclosure is not limited thereto, and the wavelength regions of light emitted from the light emitting structures OL-B1, OL-B2 and OL-B3 may be different from each other. For example, the light emitting element ED-BT including the multiple light emitting structures OL-B1, OL-B2 and OL-B3 emitting light in different wavelength regions may emit white light.

Between neighboring light emitting structures OL-B1, OL-B2 and OL-B3, charge generating layers CGL1 and CGL2 may be provided. The charge generating layers CGL1 and CGL2 may include a p-type (e.g., P) charge generating layer and/or an n-type (e.g., N) charge generating layer.

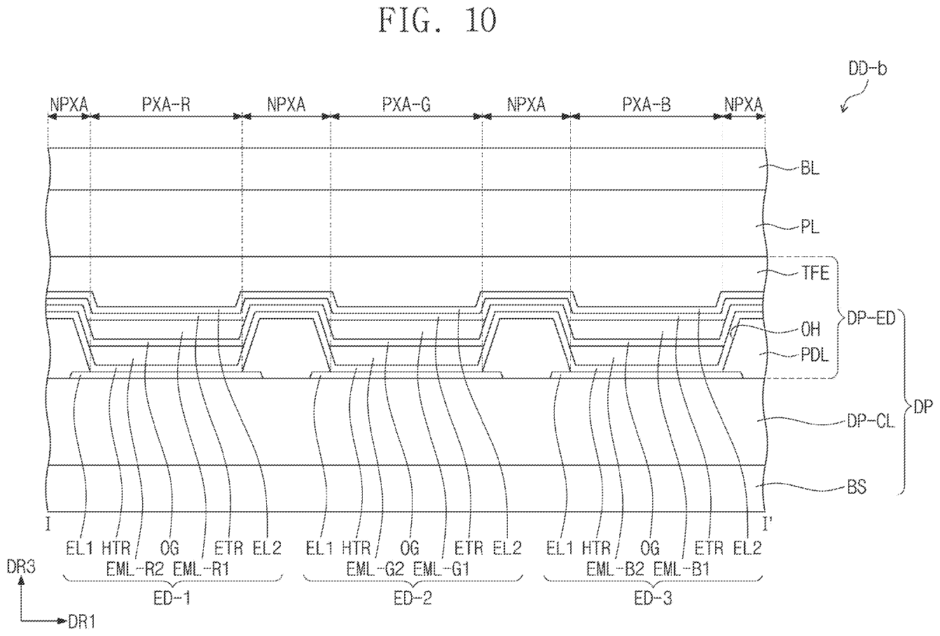

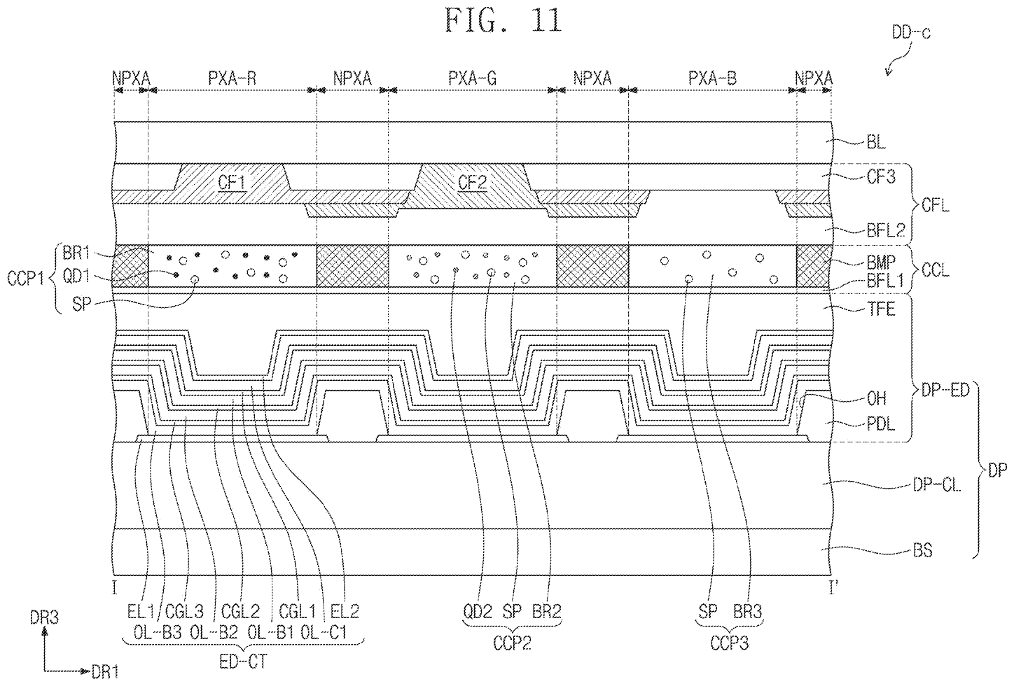

Referring to FIG. 10, a display device DD-b according to one or more embodiments may include light emitting elements ED-1, ED-2 and ED-3, formed by stacking two emission layers. Compared to the display device DD of FIG. 2, the display device DD-b shown in FIG. 10 is different in that first to third light emitting elements ED-1, ED-2 and ED-3 each include two emission layers stacked in a thickness direction. In the first to third light emitting elements ED-1, ED-2 and ED-3, two emission layers may emit light in the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and between the first blue emission layer EML-B1 and the second blue emission layer EML-B2, an emission auxiliary part OG may be provided.

The emission auxiliary part OG may include a single layer or a multilayer. The emission auxiliary part OG may include a charge generating layer. For example, the emission auxiliary part OG may include an electron transport region, a charge generating layer, and a hole transport region stacked in order. The emission auxiliary part OG may be provided as a common layer in all of the first to third light emitting elements ED-1, ED-2 and ED-3. However, one or more embodiments of the present disclosure is not limited thereto, and the emission auxiliary part OG may be patterned and provided in an opening portion OH defined in a pixel definition layer PDL.

The first red emission layer EML-R1, the first green emission layer EML-G1 and the first blue emission layer EML-B1 may be provided between the emission auxiliary part OG and the electron transport region ETR. The second red emission layer EML-R2, the second green emission layer EML-G2 and the second blue emission layer EML-B2 may be provided between the hole transport region HTR and the emission auxiliary part OG.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In one or more embodiments, an optical auxiliary layer PL may be provided on a display element layer DP-ED. The optical auxiliary layer PL may include a polarization layer. The optical auxiliary layer PL may be provided on a display panel DP and may suitably control reflected light at the display panel DP by external light. In some embodiments, the optical auxiliary layer PL may not be provided (may be omitted) from the display device according to one or more embodiments.

Different from FIG. 9 and FIG. 10, a display device DD-c in FIG. 11 is shown to include four light emitting structures OL-B1, OL-B2, OL-B3 and OL-C1. A light emitting element ED-CT may include oppositely provided first electrode EL1 and second electrode EL2, and first to fourth light emitting structures OL-B1, OL-B2, OL-B3 and OL-C1 stacked in order in a thickness direction between the first electrode EL1 and the second electrode EL2. At least one of the first to fourth light emitting structures OL-B1, OL-B2, OL-B3 and OL-C1 may include the amine compound of one or more embodiments.

Between the first to fourth light emitting structures OL-B1, OL-B2, OL-B3 and OL-C1, charge generating layers CGL1, CGL2 and CGL3 may be provided. The charge generating layers CGL1, CGL2 and CGL3 provided between the neighboring light emitting structures OL-B1, OL-B2, OL-B3 and OL-C1 may include a p-type charge generating layer and/or an n-type charge generating layer.

Of the four light emitting structures, the first to third light emitting structures OL-B1, OL-B2 and OL-B3 may emit blue light, and the fourth light emitting structure OL-C1 may emit green light. However, one or more embodiments of the present disclosure is not limited thereto, and the first to fourth light emitting structures OL-B1, OL-B2, OL-B3 and OL-C1 may emit different wavelengths of light.

Hereinafter, referring to embodiments and comparative embodiments, the amine compound according to one or more embodiments and the light emitting element according to one or more embodiments of the present disclosure will be explained in more detail. However, the embodiments below are illustrations to assist the understanding of the present disclosure, but the scope of the present disclosure is not limited thereto.

### Examples

### 1. Synthesis of Amine Compounds of Embodiments

The synthetic method of the amine compound according to one or more embodiments will be explained by illustrating the synthetic methods of Compounds 1, 2, 18, 19, 24, 28, 42, 43, 59, 60, 65, 68, 124, 125, 141, 142, 148, 151, 182, 183, 199, 200, 205, 209, 223, 227 and 239. However, the synthetic methods of the amine compounds explained hereinafter are examples, and the synthetic method of the amine compound according to one or more embodiments of the present disclosure is not limited to the Examples below.

First, the synthetic methods of Intermediates C1 to C7, used in the synthetic methods of the amine compounds described below, will be explained.

### Synthesis of Intermediate C1

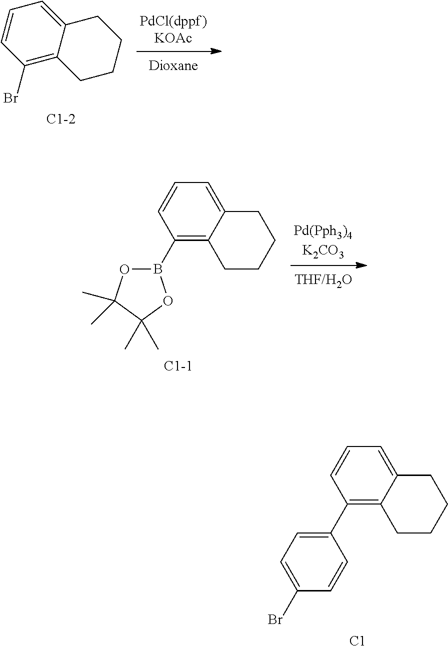 [SMILES: Brc1cccc2c1CCCC2 ; CC1(C)OB(c2cccc3c2CCCC3)OC1(C)C ; Brc1ccc(-c2cccc3c2CCCC3)cc1 ; "C1"]

Intermediate C1-2 (20 mmol, 1 eq), bis(pinacolato) diboron (40 mmol, 2 eq), PdCl₂(dppf) (0.1 mmol, 0.05 eq), KOAc (60 mmol, 3 eq), and dioxane (200 ml) were put in 1-neck-round flask and stirred at about 80° C. for about 24 hours. After finishing the reaction, the reaction product was worked-up using ether/H₂O, and separated by column chromatography using methylene chloride/hexane (MC/Hex)=1/3 (Intermediate C1-1, 14 mmol, yield=70%).

Intermediate C1-1 (14 mmol, 1 eq), 1-bromo-4-iodobenzene (14 mmol, 1 eq), Pd(Pph₃)₄ (0.7 mmol, 0.05 eq), K₂CO₃ (42 mmol, 3 eq), and THF/H₂O (200/40 ml) were put and stirred at about 80° C. for about 24 hours. After finishing the reaction, the reaction product was worked-up using ether/H₂O, and separated by column chromatography using MC/Hex=1/10 (Intermediate C1, 11.2 mmol, yield=80%).

### Synthesis of Intermediate C2

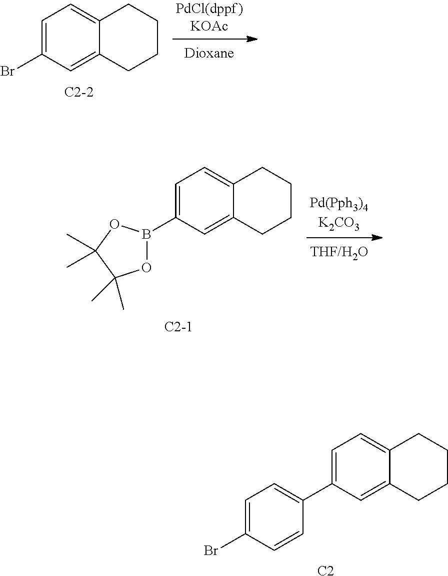

Intermediate C2-2 (20 mmol, 1 eq), bis(pinacolato) diboron (40 mmol, 2 eq), PdCl₂(dppf) (0.1 mmol, 0.05 eq), KOAc (60 mmol, 3 eq), and dioxane (200 ml) were put in 1-neck-round flask and stirred at about 80° C. for about 24 hours. After finishing the reaction, the reaction product was worked-up using ether/H₂O, and separated by column chromatography using MC/Hex=1/3 (Intermediate C2-1, 16 mmol, yield=80%).

Intermediate C2-1 (16 mmol, 1 eq), 1-bromo-4-iodobenzene (16 mmol, 1 eq), Pd(Pph₃)₄ (0.8 mmol, 0.05 eq), K₂CO₃ (48 mmol, 3 eq), and THF/H₂O (200/40 ml) were put and stirred at about 80° C. for about 24 hours. After finishing the reaction, the reaction product was worked-up using ether/H₂O, and separated by column chromatography using MC/Hex=1/10 (Intermediate C2, 12.8 mmol, yield=80%).

### Synthesis of Intermediate C3

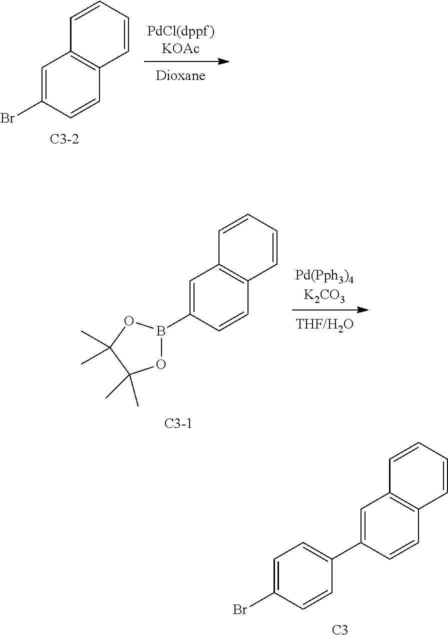

Intermediate C3-2 (20 mmol, eq), bis(pinacolato) diboron mmol, 2 eq), PdCl₂(dppf) (0.1 mmol, 0.05 eq), KOAc (60 mmol, 3 eq), and dioxane (200 ml) were put in 1-neck-round flask and stirred at about 80° C. for about 24 hours. After finishing the reaction, the reaction product was worked-up using ether/H₂O, and separated by column chromatography using MC/Hex=1/3 (Intermediate C3-1, 10 mmol, yield=50%).

Intermediate C3-1 (10 mmol, 1 eq), 1-amino-4-bromobenzene (10 mmol, 1 eq), Pd(Pph₃)₄ (0.5 mmol, 0.05 eq), K₂CO₃ (30 mmol, 3 eq), and THF/H₂O (150/30 ml) were put and stirred at about 80° C. for about 24 hours. After finishing the reaction, the reaction product was worked-up using ether/H₂O, and separated by column chromatography using MC/Hex=1/3 (Intermediate C3, 8 mmol, yield=80%).

### Synthesis of Intermediate C4

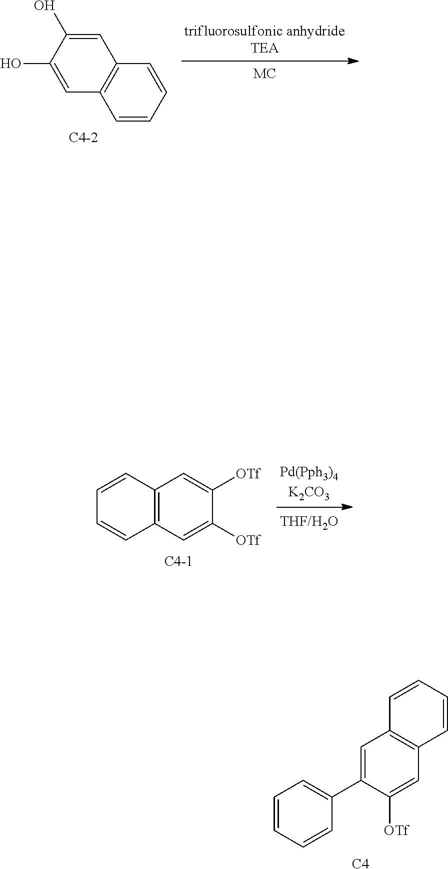

Intermediate C4-2 (20 mmol, 1 eq), trifluorosulfonic anhydride (80 mmol, 4 eq), triethanolamine (TEA, 60 mmol, 3 eq), and MC (200 ml) were put in 1-neck-round flask and stirred at about 0° C. for about 2 hours. After finishing the reaction, the reaction product was worked-up using ether/H₂O, and separated by column chromatography using Hx (Intermediate C4-1, 18 mmol, yield=90%).

Intermediate C4-1 (18 mmol, 1 eq), 1-amino-4-bromobenzene (18 mmol, 1 eq), Pd(Pph₃)₄ (0.9 mmol, 0.05 eq), K₂CO₃ (54 mmol, 3 eq), and THF/H₂O (200/40 ml) were put and stirred at about 80° C. for about 24 hours. After finishing the reaction, the reaction product was worked-up using ether/H₂O, and separated by column chromatography using Hx (Intermediate C4, 9 mmol, yield=50%).

### Synthesis of Intermediate C5

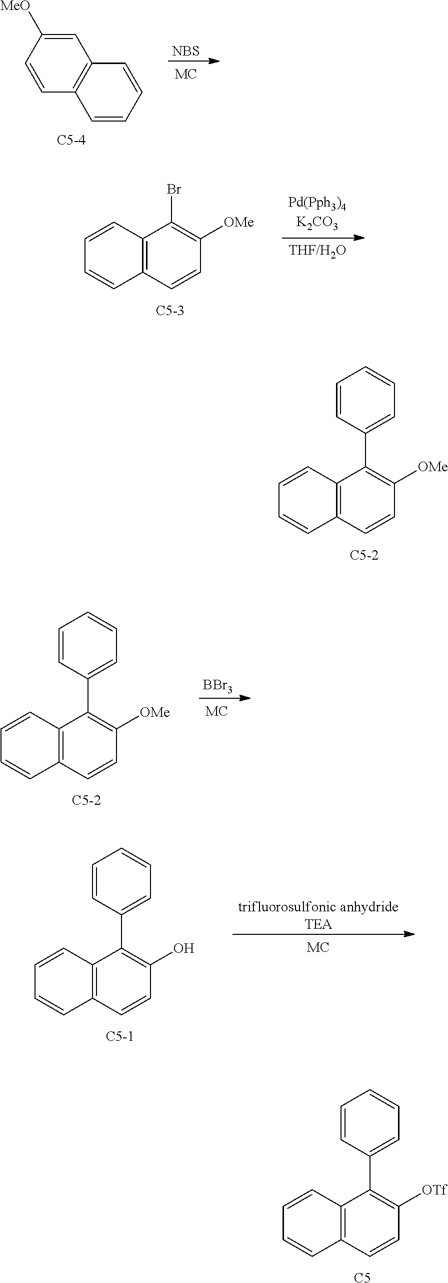

Intermediate C5-4 (50 mmol, 1 eq), N-bromosuccinimide (NBS, 50 mmol, 1 eq), and MC (400 ml) were put in 1-neck-round flask and stirred at about 70° C. for about 4 hours. After finishing the reaction, the reaction product was worked-up using ether/H₂O, and separated by column chromatography using Hx (Intermediate C5-3, 45 mmol, yield=90%).

Intermediate C5-3 (45 mmol, 1 eq), phenyl boronic acid (45 mmol, 1 eq), Pd(Pph₃)₄ (2.25 mmol, 0.05 eq), K₂CO₃ (135 mmol, 3 eq), and THF/H₂O (400/80 ml) were put and stirred at about 80° C. for about 24 hours. After finishing the reaction, the reaction product was worked-up using ether/H₂O, and separated by column chromatography using Hx (Intermediate C5-2, 36 mmol, yield=80%).

Intermediate C5-2 (36 mmol, 1 eq), BBr₃ (36 mmol, 1 eq), and MC (400 ml) were put in 1-neck-round flask and stirred at about 0° C. for about 4 hours. After finishing the reaction, the reaction product was worked-up using ether/H₂O, and separated by column chromatography using MC/Hx=1/3 (Intermediate C5-1, 31.5 mmol, yield=90%).

Intermediate C5-1 (31.5 mmol, 1 eq), trifluorosulfonic anhydride (63 mmol, 2 eq), TEA (63 mmol, 2 eq), and MC (400 ml) were put in 1-neck-round flask and stirred at about 0° C. for about 2 hours. After finishing the reaction, the reaction product was worked-up using ether/H₂O, and separated by column chromatography using Hx (Intermediate C5, 26.8 mmol, yield=85%).

### Synthesis of Intermediate C6

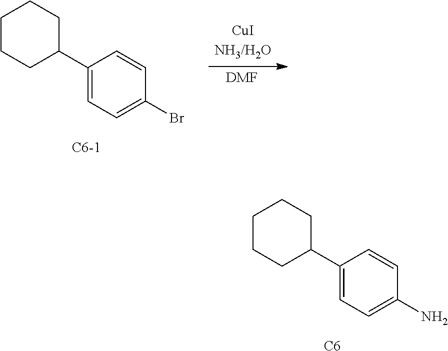

Intermediate C6-1 (100 mmol, 1 eq), CuI (100 mmol, 1 eq), NH₃/H₂O (70 ml), and dimethylformamide (DMF, 70 ml) were put in 1-neck-round flask and stirred at about 110° C. for about 6 hours. After finishing the reaction, the reaction product was worked-up using ether/H₂O, and separated by column chromatography using MC/Hx=1/3 (Intermediate C6, 40 mmol, yield=40%).

### Synthesis of Intermediate C7

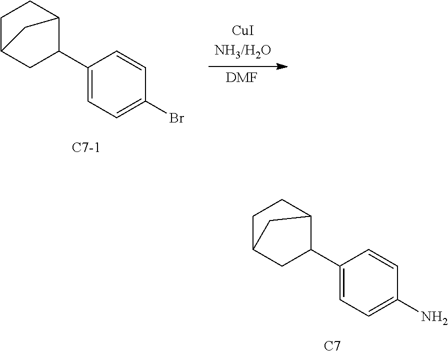

Intermediate C7-1 (100 mmol, 1 eq), CuI (100 mmol, 1 eq), NH₃/H₂O (70 ml), and DMF (70 ml) were put in 1-neck-round flask and stirred at about 110° C. for about 6 hours. After finishing the reaction, the reaction product was worked-up using ether/H₂O, and separated by column chromatography using MC/Hx=1/3 (Intermediate C7, 35 mmol, yield=35%).

### (1) Synthesis of Amine Compound 1

Amine Compound 1 according to one or more embodiments may be synthesized by, for example, the step of Reaction 1 below.

### Reaction 1

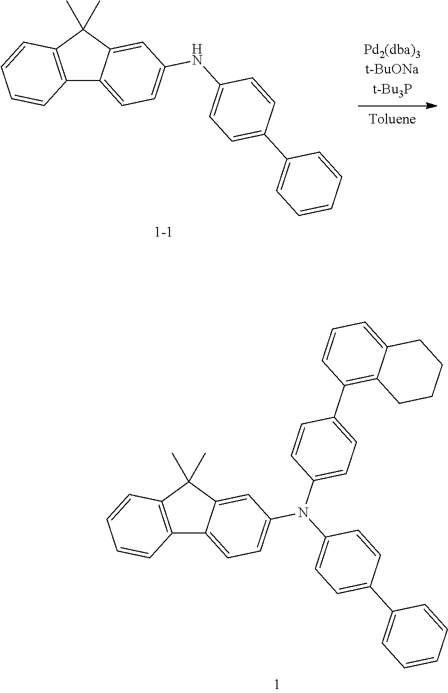 [SMILES: CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc(-c4cccc5c4CCCC5)cc3)cc21]

Intermediate 1-1 (11 mmol, 1.1 eq), Intermediate C1 (10 mmol, 1 eq), Pd₂(dba)₃ (0.5 mmol, 0.05 eq), t-BuONa (20 mmol, 2 eq), t-Bu₃P (1 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/7. 7.6 mmol of Compound 1 was obtained (yield=76%).

### (2) Synthesis of Amine Compound 2

Amine Compound 2 according to one or more embodiments may be synthesized by, for example, the steps of Reaction 2 below.

### Reaction 2

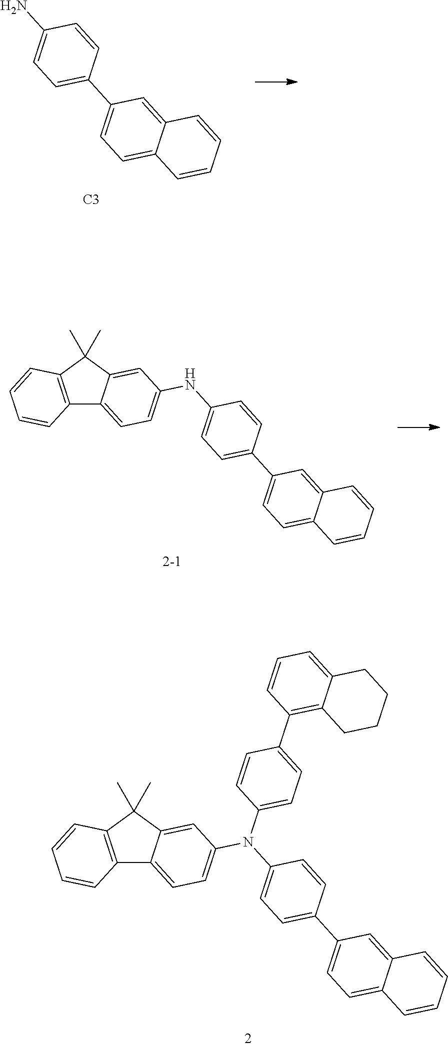 [SMILES: Nc1ccc(-c2ccc3ccccc3c2)cc1 ; "C3" ; CC1(C)c2ccccc2-c2ccc(Nc3ccc(-c4ccc5ccccc5c4)cc3)cc21 ; CC1(C)c2ccccc2-c2ccc(N(c3ccc(-c4ccc5ccccc5c4)cc3)c3ccc(-c4cccc5c4CCCC5)cc3)cc21]

Intermediate C3 (22 mmol, 1.1 eq), 2-bromo-9,9-dimethylfluorene (20 mmol, 1 eq), Pd₂(dba)₃ (1 mmol, 0.05 eq), t-BuONa (40 mmol, 2 eq), t-Bu₃P (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16 mmol of Intermediate 2-1 was obtained (yield=80%).

Intermediate 2-1 (16 mmol, 1.1 eq), Intermediate C1 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/8. 10.8 mmol of Compound 2 was obtained (yield=75%).

### (3) Synthesis of Amine Compound 18

Amine Compound 18 according to one or more embodiments may be synthesized by, for example, the steps of Reaction 3 below.

### Reaction 3

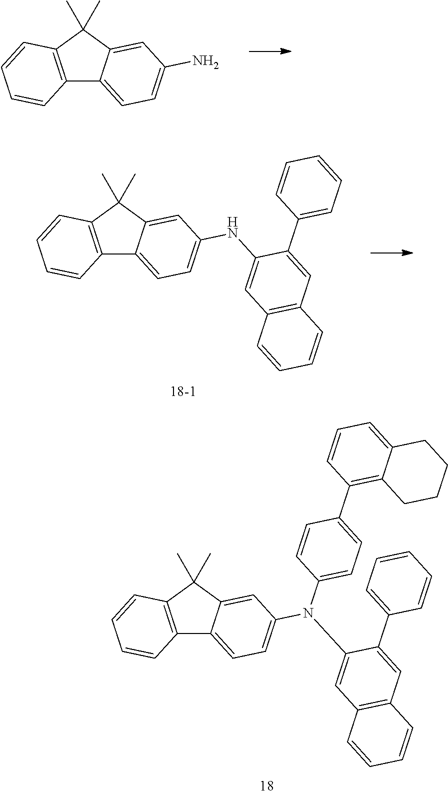 [SMILES: CC1(C)c2ccccc2-c2ccc(N)cc21 ; CC1(C)c2ccccc2-c2ccc(Nc3cc4ccccc4cc3-c3ccccc3)cc21 ; CC1(C)c2ccccc2-c2ccc(N(c3ccc(-c4cccc5c4CCCC5)cc3)c3cc4ccccc4cc3-c3ccccc3)cc21]

2-amino-9,9-dimethylfluorene (22 mmol, 1.1 eq), Intermediate C4 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 18-1 was obtained (yield=50%).

Intermediate 18-1 (10 mmol, 1.1 eq), Intermediate C1 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 6.8 mmol of Compound 18 was obtained (yield=68%).

### (4) Synthesis of Amine Compound 19

Amine Compound 19 according to one or more embodiments may be synthesized by, for example, the steps of Reaction 4 below.

### Reaction 4

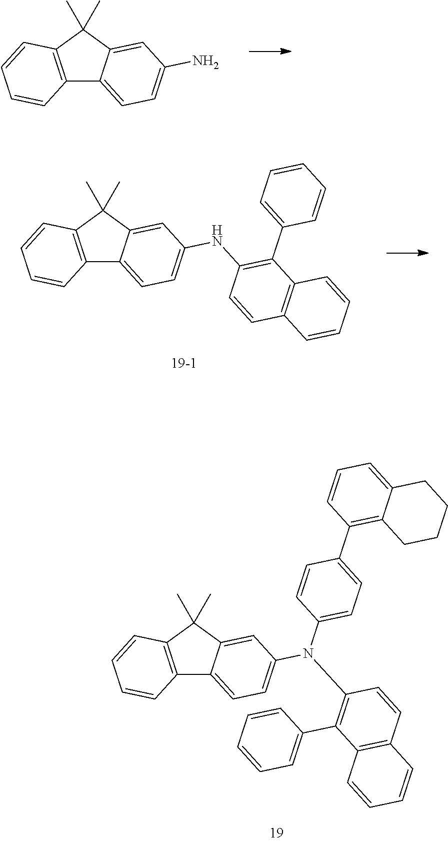 [SMILES: CC1(C)c2ccccc2-c2ccc(N)cc21 ; CC1(C)c2ccccc2-c2ccc(Nc3ccc4ccccc4c3-c3ccccc3)cc21 ; CC1(C)c2ccccc2-c2ccc(N(c3ccc(-c4cccc5c4CCCC5)cc3)c3ccc4ccccc4c3-c3ccccc3)cc21]

2-amino-9,9-dimethylfluorene (22 mmol, 1.1 eq), Intermediate C5 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 19-1 was obtained (yield=50%).

Intermediate 19-1 (10 mmol, 1.1 eq), Intermediate C1 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 6.8 mmol of Compound 19 was obtained (yield=68%).

### (5) Synthesis of Amine Compound 24

Amine Compound 24 according to one or more embodiments may be synthesized by, for example, the steps of Reaction 5 below.

### Reaction 5

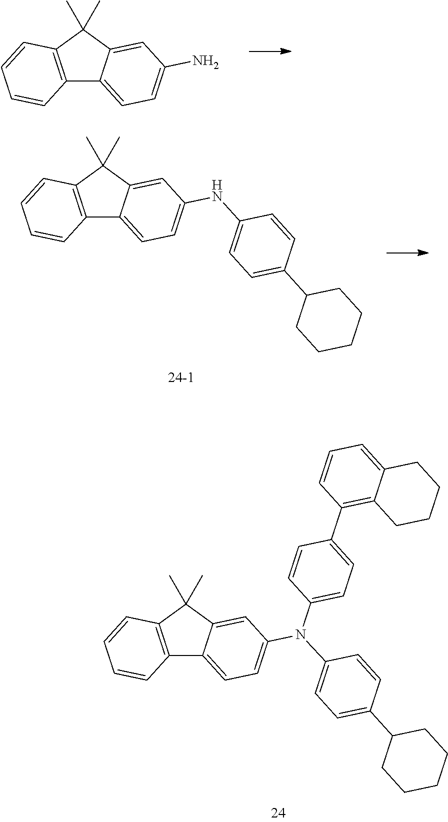

2-amino-9,9-dimethylfluorene (22 mmol, 1.1 eq), Intermediate C6 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16 mmol of Intermediate 24-1 was obtained (yield=80%).

Intermediate 24-1 (16 mmol, 1.1 eq), Intermediate C1 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 10.15 mmol of Compound 24 was obtained (yield=70%).

### (6) Synthesis of Amine Compound 28

Amine Compound 28 according to one or more embodiments may be synthesized by, for example, the steps of Reaction 6 below.

### Reaction 6

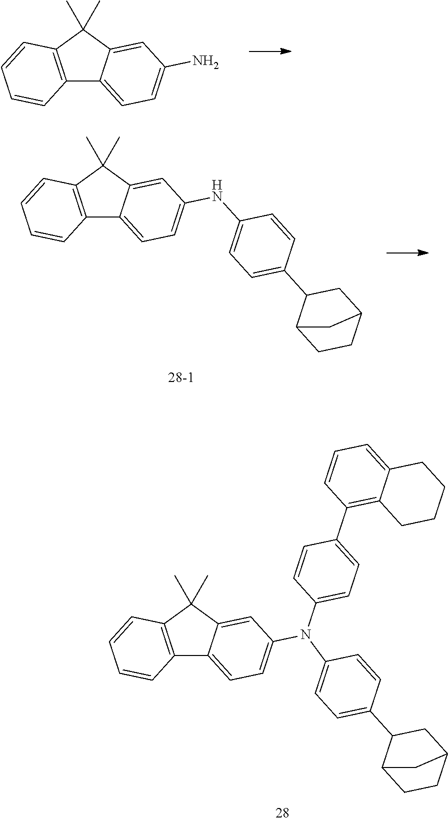

2-amino-9,9-dimethylfluorene (22 mmol, 1.1 eq), Intermediate C7 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16.8 mmol of Intermediate 28-1 was obtained (yield=84%).

Intermediate 28-1 (16 mmol, 1.1 eq), Intermediate C1 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 10.85 mmol of Compound 28 was obtained (yield=75%).

### (7) Synthesis of Amine Compound 42

Amine Compound 42 according to one or more embodiments may be synthesized by, for example, the step of Reaction 7 below.

### Reaction 7

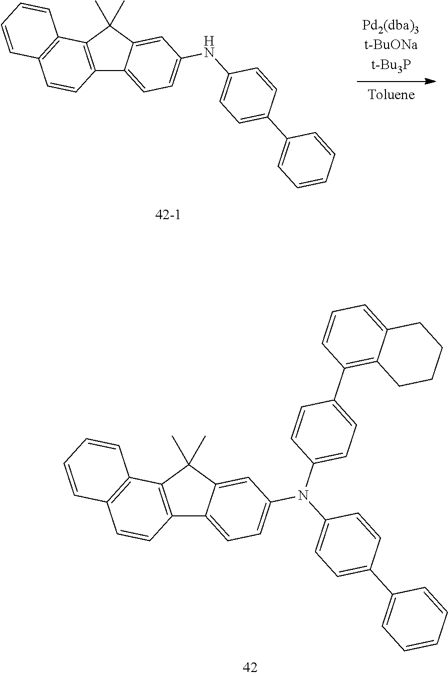 [SMILES: CC1(C)c2cc(Nc3ccc(-c4ccccc4)cc3)ccc2-c2ccc3ccccc3c21 ; CC1(C)c2cc(N(c3ccc(-c4ccccc4)cc3)c3ccc(-c4cccc5c4CCCC5)cc3)ccc2-c2ccc3ccccc3c21]

Intermediate 42-1 (11 mmol, 1.1 eq), Intermediate C1 (10 mmol, 1 eq), Pd₂(dba)₃ (0.5 mol, 0.05 eq), t-BuONa (20 mmol, 2 eq), t-Bu₃P (1 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/7. 6.9 mmol of Compound 42 was obtained (yield=69%).

### (8) Synthesis of Amine Compound 43

Amine Compound 43 according to one or more embodiments may be synthesized by, for example, the steps of Reaction 8 below.

### Reaction 8

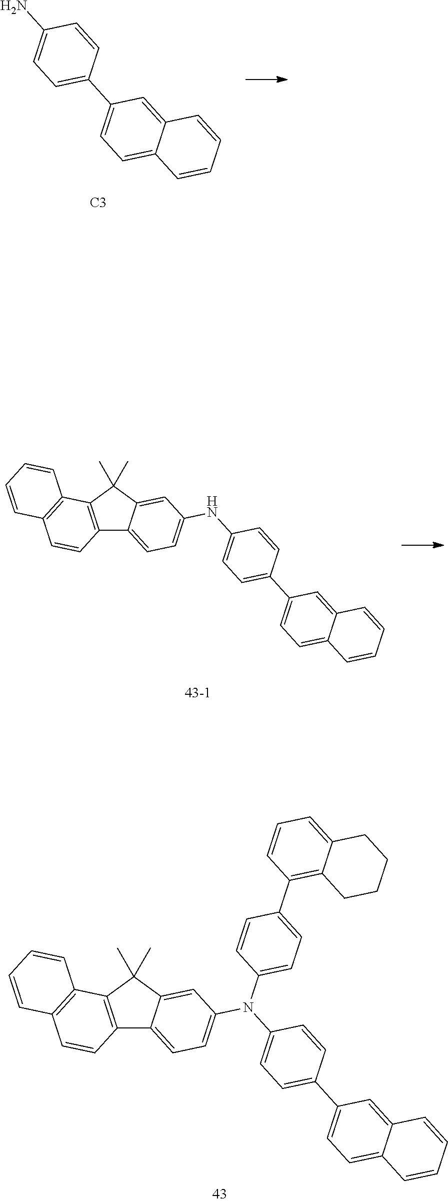 [SMILES: Nc1ccc(-c2ccc3ccccc3c2)cc1 ; "C3" ; CC1(C)c2cc(Nc3ccc(-c4ccc5ccccc5c4)cc3)ccc2-c2ccc3ccccc3c21 ; CC1(C)c2cc(N(c3ccc(-c4ccc5ccccc5c4)cc3)c3ccc(-c4cccc5c4CCCC5)cc3)ccc2-c2ccc3ccccc3c21]

Intermediate C3 (22 mmol, 1.1 eq), 9-bromo-11,11-dimethyl-11H-benzo[a]fluorene (20 mmol, 1 eq), Pd₂(dba)₃ (1 mmol, 0.05 eq), t-BuONa (40 mmol, 2 eq), t-Bu₃P (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/4. 13.6 mmol of Intermediate 43-1 was obtained (yield=68%).

Intermediate 43-1 (13.6 mmol, 1.1 eq), Intermediate C1 (12.36 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/8. 9.3 mmol of Compound 43 was obtained (yield=75%).

### (9) Synthesis of Amine Compound 59

Amine Compound 59 according to one or more embodiments may be synthesized by, for example, the steps of Reaction 9 below.

### Reaction 9

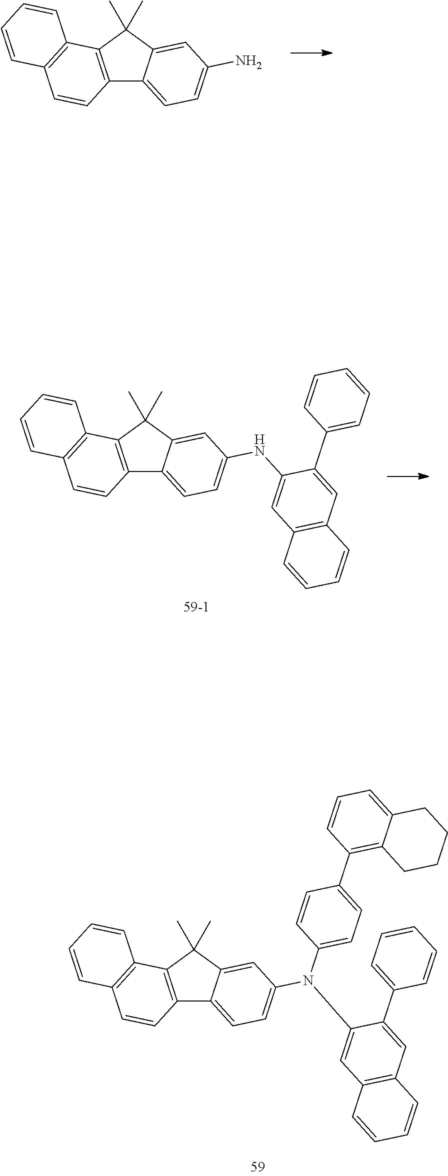 [SMILES: CC1(C)c2cc(N)ccc2-c2ccc3ccccc3c21 ; CC1(C)c2cc(Nc3cc4ccccc4cc3-c3ccccc3)ccc2-c2ccc3ccccc3c21 ; CC1(C)c2cc(N(c3ccc(-c4cccc5c4CCCC5)cc3)c3cc4ccccc4cc3-c3ccccc3)ccc2-c2ccc3ccccc3c21]

11,11-dimethyl-11H-benzo[a]fluoren-9-amine (22 mmol, 1.1 eq), Intermediate C4 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 9 mmol of Intermediate 59-1 was obtained (yield=45%).

Intermediate 59-1 (10 mmol, 1.1 eq), Intermediate C1 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/8. 6.3 mmol of Compound 59 was obtained (yield=70%).

### (10) Synthesis of Amine Compound 60

Amine Compound 60 according to one or more embodiments may be synthesized by, for example, the steps of Reaction 10 below.

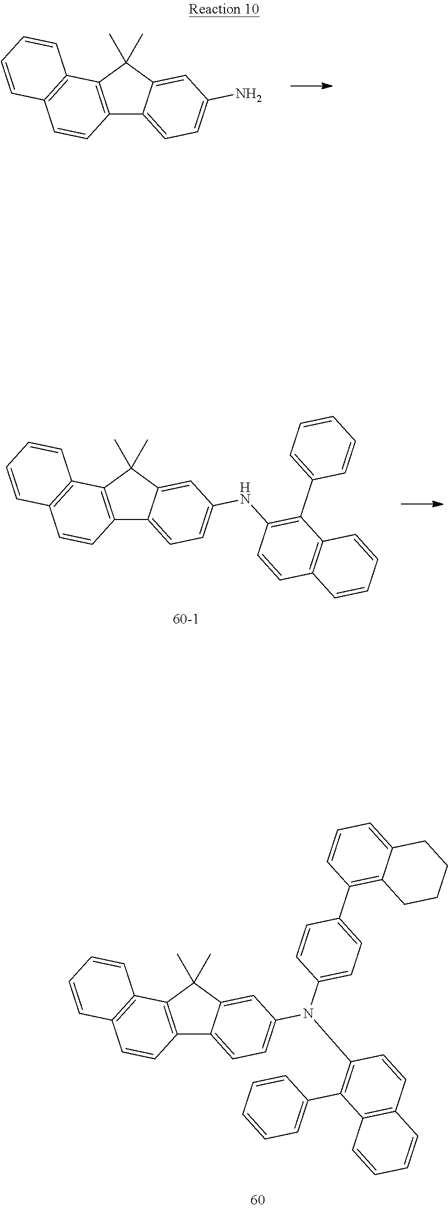 [SMILES: CC1(C)c2cc(N)ccc2-c2ccc3ccccc3c21 ; CC1(C)c2cc(Nc3ccc4ccccc4c3-c3ccccc3)ccc2-c2ccc3ccccc3c21 ; CC1(C)c2cc(N(c3ccc(-c4cccc5c4CCCC5)cc3)c3ccc4ccccc4c3-c3ccccc3)ccc2-c2ccc3ccccc3c21]

11,11-dimethyl-11H-benzo[a]fluoren-9-amine (22 mmol, 1.1 eq), Intermediate C5 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 60-1 was obtained (yield=50%).

Intermediate 60-1 (10 mmol, 1.1 eq), Intermediate C1 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 6.8 mmol of Compound 60 was obtained (yield=68%).

### (11) Synthesis of Amine Compound 65

Amine Compound 65 according to one or more embodiments may be synthesized by, for example, the steps of Reaction 11 below.

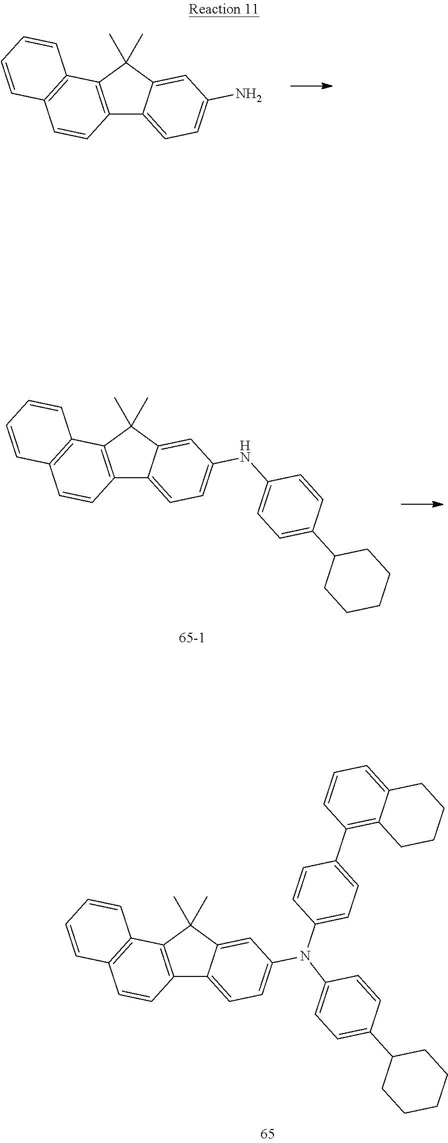 [SMILES: CC1(C)c2cc(N)ccc2-c2ccc3ccccc3c21 ; CC1(C)c2cc(Nc3ccc(C4CCCCC4)cc3)ccc2-c2ccc3ccccc3c21 ; CC1(C)c2cc(N(c3ccc(-c4cccc5c4CCCC5)cc3)c3ccc(C4CCCCC4)cc3)ccc2-c2ccc3ccccc3c21]

11,11-dimethyl-11H-benzo[a]fluoren-9-amine (22 mmol, 1.1 eq), Intermediate C6 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16 mmol of Intermediate 65-1 was obtained (yield=80%).

Intermediate 65-1 (16 mmol, 1.1 eq), Intermediate C1 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 10.15 mmol of Compound 65 was obtained (yield=70%).

### (12) Synthesis of Amine Compound 68

Amine Compound 68 according to one or more embodiments may be synthesized by, for example, the steps of Reaction 12 below.

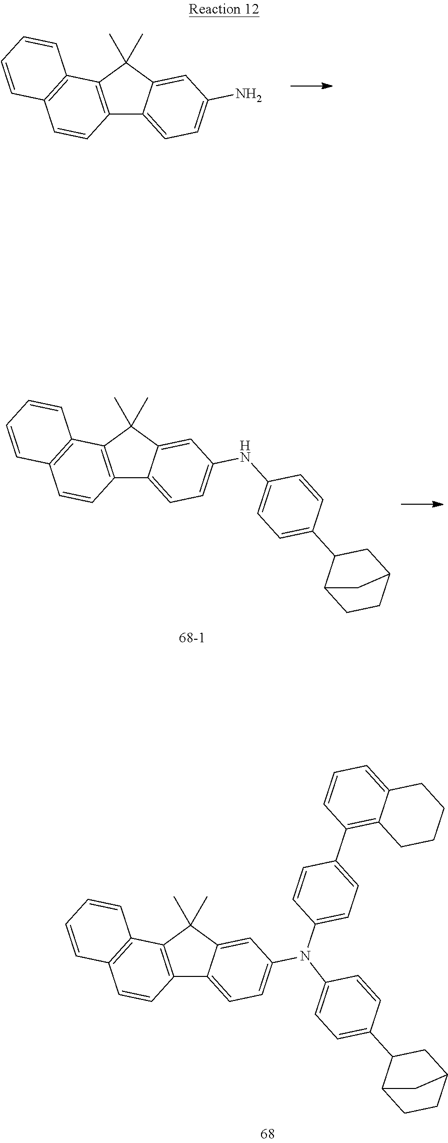 [SMILES: CC1(C)c2cc(N)ccc2-c2ccc3ccccc3c21 ; CC1(C)c2cc(Nc3ccc(C4CC5CCC4C5)cc3)ccc2-c2ccc3ccccc3c21 ; CC1(C)c2cc(N(c3ccc(-c4cccc5c4CCCC5)cc3)c3ccc(C4CC5CCC4C5)cc3)ccc2-c2ccc3ccccc3c21]

11,11-dimethyl-11H-benzo[a]fluoren-9-amine (22 mmol, 1.1 eq), Intermediate C7 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16.8 mmol of Intermediate 68-1 was obtained (yield=84%).

Intermediate 68-1 (16 mmol, 1.1 eq), Intermediate C1 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 10.85 mmol of Compound 68 was obtained (yield=75%).

### (13) Synthesis of Amine Compound 124

Amine Compound 124 according to one or more embodiments may be synthesized by, for example, the step of Reaction 13 below.

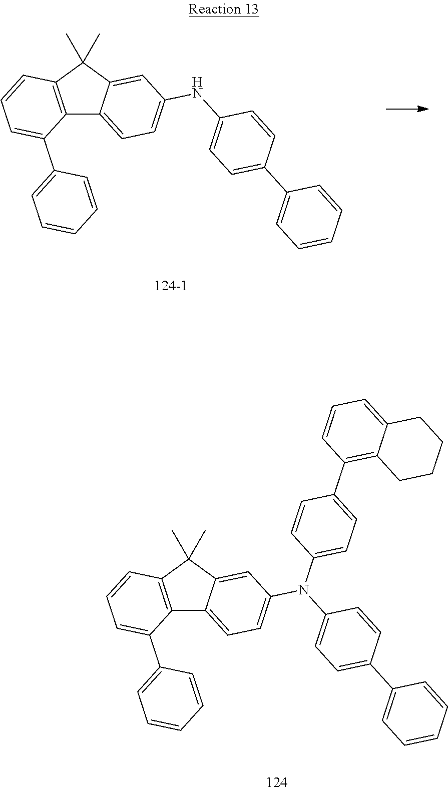 [SMILES: CC1(C)c2cc(Nc3ccc(-c4ccccc4)cc3)ccc2-c2c(-c3ccccc3)cccc21 ; CC1(C)c2cc(N(c3ccc(-c4ccccc4)cc3)c3ccc(-c4cccc5c4CCCC5)cc3)ccc2-c2c(-c3ccccc3)cccc21]

Intermediate 124-1 (11 mmol, 1.1 eq), Intermediate C1 (10 mmol, 1 eq), Pd₂(dba)₃ (0.5 mol, 0.05 eq), t-BuONa (20 mmol, 2 eq), t-Bu₃P (1 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/7. 7.6 mmol of Compound 124 was obtained (yield=76%).

### (14) Synthesis of Amine Compound 125

Amine Compound 125 according to one or more embodiments may be synthesized by, for example, the steps of Reaction 14 below.

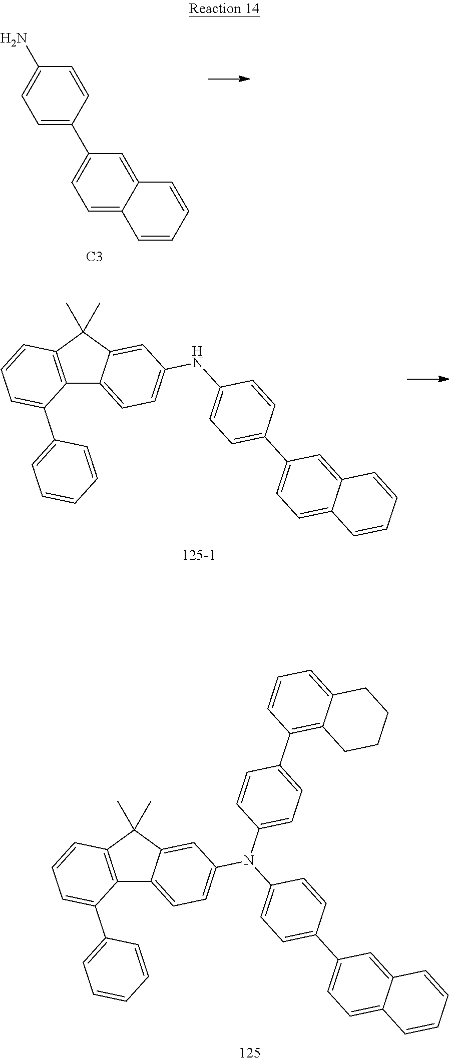 [SMILES: Nc1ccc(-c2ccc3ccccc3c2)cc1 ; "C3" ; CC1(C)c2cc(Nc3ccc(-c4ccc5ccccc5c4)cc3)ccc2-c2c(-c3ccccc3)cccc21 ; CC1(C)c2cc(N(c3ccc(-c4ccc5ccccc5c4)cc3)c3ccc(-c4cccc5c4CCCC5)cc3)ccc2-c2c(-c3ccccc3)cccc21]

Intermediate C3 (22 mmol, 1.1 eq), 2-bromo-9,9-dimethyl-5-phenyl-9H-fluorene (20 mmol, 1 eq), Pd₂(dba)₃ (1 mmol, 0.05 eq), t-BuONa (40 mmol, 2 eq), t-Bu₃P (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16 mmol of Intermediate 125-1 was obtained (yield=80%).

Intermediate 125-1 (16 mmol, 1.1 eq), Intermediate C1 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/8. 10.8 mmol of Compound 125 was obtained (yield=75%).

### (15) Synthesis of Amine Compound 141

Amine Compound 141 according to one or more embodiments may be synthesized by, for example, the steps of Reaction 15 below.

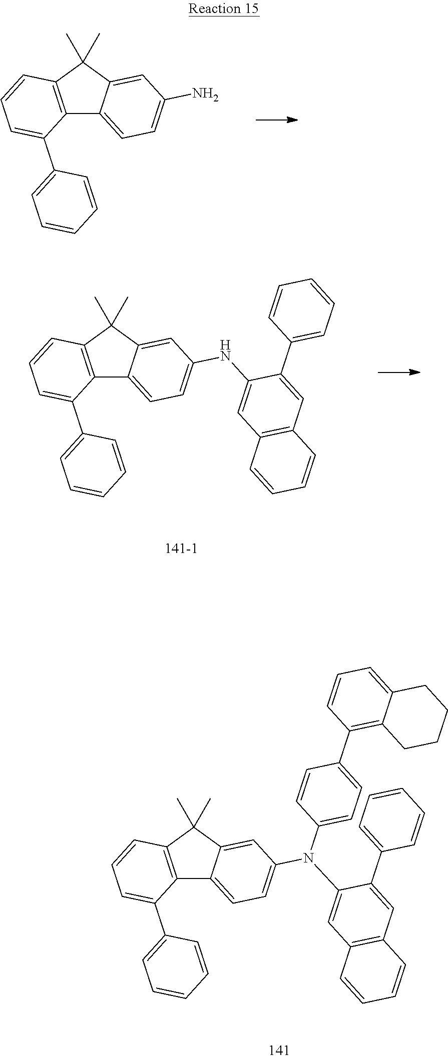 [SMILES: CC1(C)c2cc(N)ccc2-c2c(-c3ccccc3)cccc21 ; CC1(C)c2cc(Nc3cc4ccccc4cc3-c3ccccc3)ccc2-c2c(-c3ccccc3)cccc21 ; CC1(C)c2cc(N(c3ccc(-c4cccc5c4CCCC5)cc3)c3cc4ccccc4cc3-c3ccccc3)ccc2-c2c(-c3ccccc3)cccc21]

9,9-dimethyl-5-phenyl-9H-fluoren-2-amine (22 mmol, 1.1 eq), Intermediate C4 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 141-1 was obtained (yield=50%).

Intermediate 141-1 (10 mmol, 1.1 eq), Intermediate C1 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 6.8 mmol of Compound 141 was obtained (yield=68%).

### (16) Synthesis of Amine Compound 142

Amine Compound 142 according to one or more embodiments may be synthesized by, for example, the steps of Reaction 16 below.

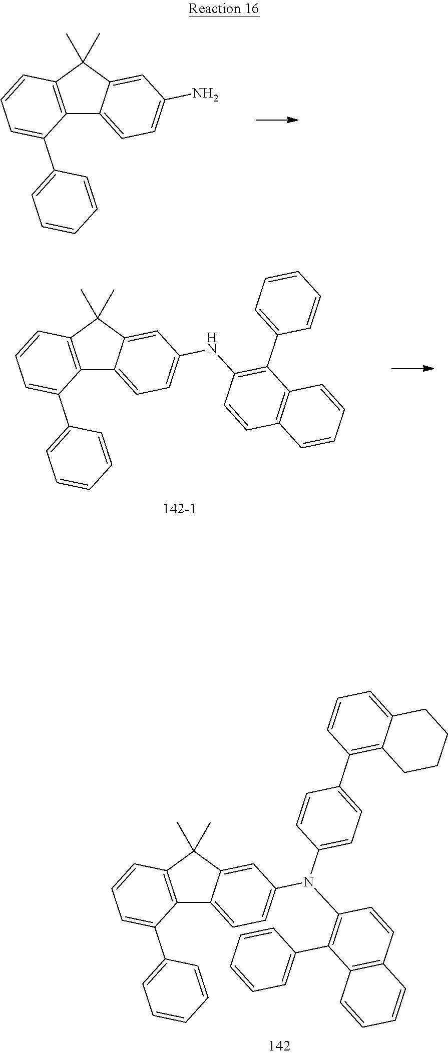 [SMILES: CC1(C)c2cc(N)ccc2-c2c(-c3ccccc3)cccc21 ; CC1(C)c2cc(Nc3ccc4ccccc4c3-c3ccccc3)ccc2-c2c(-c3ccccc3)cccc21 ; CC1(C)c2cc(N(c3ccc(-c4cccc5c4CCCC5)cc3)c3ccc4ccccc4c3-c3ccccc3)ccc2-c2c(-c3ccccc3)cccc21]

9,9-dimethyl-5-phenyl-9H-fluoren-2-amine (22 mmol, 1.1 eq), Intermediate C5 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 142-1 was obtained (yield=50%).

Intermediate 142-1 (10 mmol, 1.1 eq), Intermediate C1 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 6.8 mmol of Compound 142 was obtained (yield=68%).

### (17) Synthesis of Amine Compound 148

Amine Compound 148 according to one or more embodiments may be synthesized by, for example, the steps of Reaction 17 below.

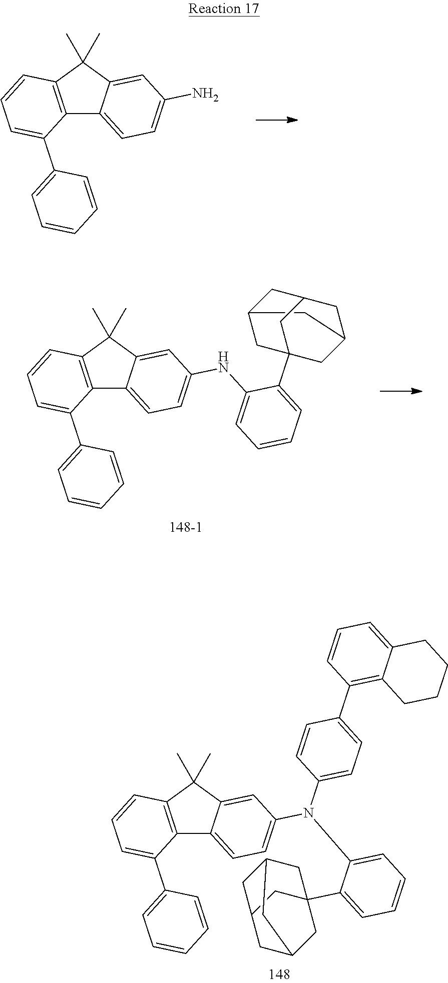 [SMILES: CC1(C)c2cc(N)ccc2-c2c(-c3ccccc3)cccc21 ; CC1(C)c2cc(Nc3ccccc3C34CC5CC(CC(C5)C3)C4)ccc2-c2c(-c3ccccc3)cccc21 ; CC1(C)c2cc(N(c3ccc(-c4cccc5c4CCCC5)cc3)c3ccccc3C34CC5CC(CC(C5)C3)C4)ccc2-c2c(-c3ccccc3)cccc21]

9,9-dimethyl-5-phenyl-9H-fluoren-2-amine (22 mmol, 1.1 eq), 1-(2-bromophenyl)adamantane (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16 mmol of Intermediate 148-1 was obtained (yield=80%).

Intermediate 148-1 (16 mmol, 1.1 eq), Intermediate C1 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 10.15 mmol of Compound 148 was obtained (yield=70%).

### (18) Synthesis of Amine Compound 151

Amine Compound 151 according to one or more embodiments may be synthesized by, for example, the steps of Reaction 18 below.

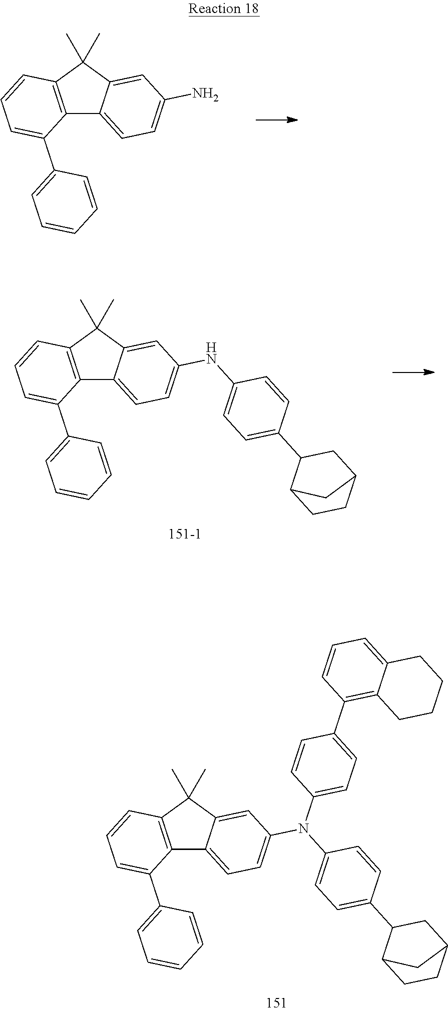 [SMILES: CC1(C)c2cc(N)ccc2-c2c(-c3ccccc3)cccc21 ; CC1(C)c2cc(Nc3ccc(C4CC5CCC4C5)cc3)ccc2-c2c(-c3ccccc3)cccc21 ; CC1(C)c2cc(N(c3ccc(-c4cccc5c4CCCC5)cc3)c3ccc(C4CC5CCC4C5)cc3)ccc2-c2c(-c3ccccc3)cccc21]

9,9-dimethyl-5-phenyl-9H-fluoren-2-amine (22 mmol, 1.1 eq), Intermediate C7 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16.8 mmol of Intermediate 151-1 was obtained (yield=84%).

Intermediate 151-1 (16 mmol, 1.1 eq), Intermediate C1 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 10.85 mmol of Compound 151 was obtained (yield=75%).

### (19) Synthesis of Amine Compound 182

Amine Compound 182 according to one or more embodiments may be synthesized by, for example, the step of Reaction 19 below.

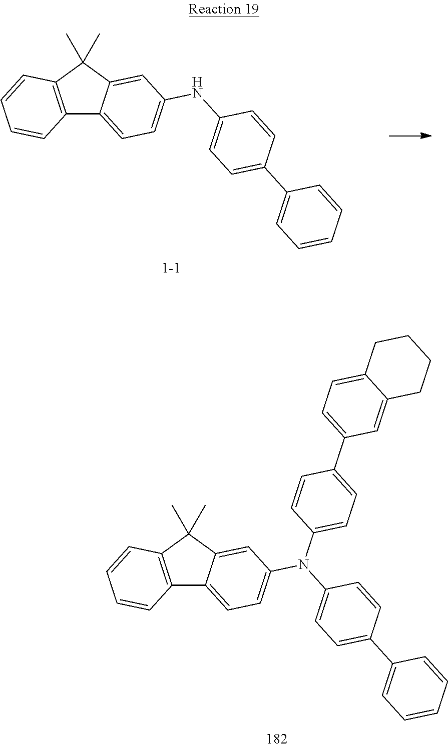 [SMILES: CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc(-c4ccc5c(c4)CCCC5)cc3)cc21]

Intermediate 1-1 (11 mmol, 1.1 eq), Intermediate C2 (10 mmol, 1 eq), Pd₂(dba)₃ (0.5 mol, 0.05 eq), t-BuONa (30 mmol, 3 eq), t-Bu₃P (1 mmol, 0.1 eq), and toluene (100 ml) were put in 1-neck-round flask and stirred at about 80° C. for about 2 hours. After finishing the reaction, the reaction product was worked-up using ether/H₂O, and separated by column chromatography using MC/Hx=1/7. 8.3 mmol of Compound 182 was obtained (yield=83%).

### (20) Synthesis of Amine Compound 183

Amine Compound 183 according to one or more embodiments may be synthesized by, for example, the steps of Reaction 20 below.

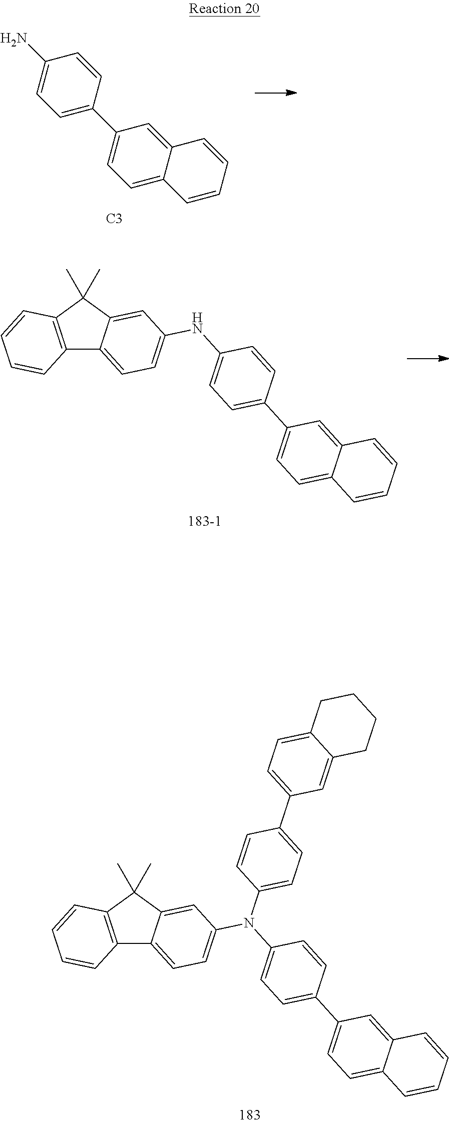 [SMILES: Nc1ccc(-c2ccc3ccccc3c2)cc1 ; "C3" ; CC1(C)c2ccccc2-c2ccc(Nc3ccc(-c4ccc5ccccc5c4)cc3)cc21 ; CC1(C)c2ccccc2-c2ccc(N(c3ccc(-c4ccc5c(c4)CCCC5)cc3)c3ccc(-c4ccc5ccccc5c4)cc3)cc21]

Intermediate C3 (22 mmol, 1.1 eq), 2-bromo-9,9-dimethylfluorene (20 mmol, 1 eq), Pd₂(dba)₃ (1 mmol, 0.05 eq), t-BuONa (40 mmol, 2 eq), t-Bu₃P (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16 mmol of Intermediate 183-1 was obtained (yield=80%).

Intermediate 183-1 (16 mmol, 1.1 eq), Intermediate C2 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/8. 10.8 mmol of Compound 183 was obtained (yield=75%).

### (21) Synthesis of Amine Compound 199

Amine Compound 199 according to one or more embodiments may be synthesized by, for example, the steps of Reaction 21 below.

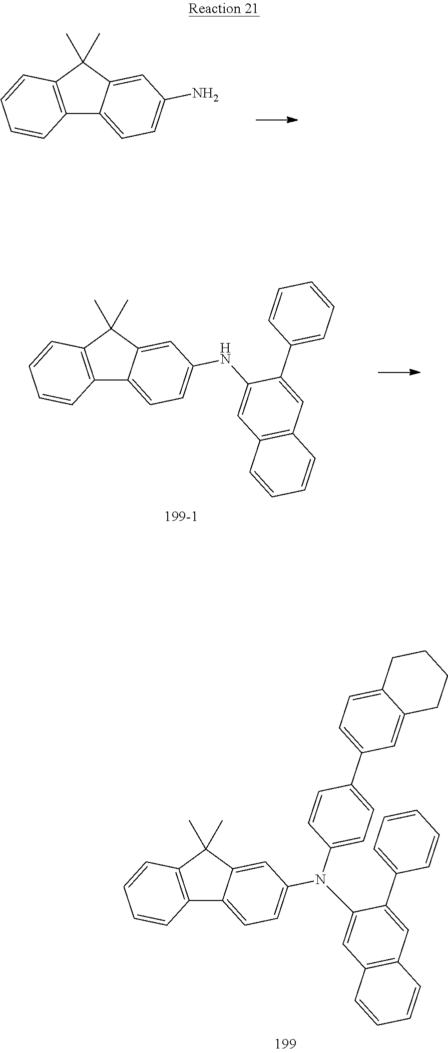 [SMILES: CC1(C)c2ccccc2-c2ccc(N)cc21 ; CC1(C)c2ccccc2-c2ccc(Nc3cc4ccccc4cc3-c3ccccc3)cc21 ; CC1(C)c2ccccc2-c2ccc(N(c3ccc(-c4ccc5c(c4)CCCC5)cc3)c3cc4ccccc4cc3-c3ccccc3)cc21]

2-amino-9,9-dimethylfluorene (22 mmol, 1.1 eq), Intermediate C4 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 199-1 was obtained (yield=50%).

Intermediate 199-1 (10 mmol, 1.1 eq), Intermediate C2 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. After finishing the reaction, the reaction product was worked-up using ether/H₂O, and separated by column chromatography using Hx. 6.8 mmol of Compound 199 was obtained (yield=68%).

### (22) Synthesis of Amine Compound 200

Amine Compound 200 according to one or more embodiments may be synthesized by, for example, the steps of Reaction 22 below.

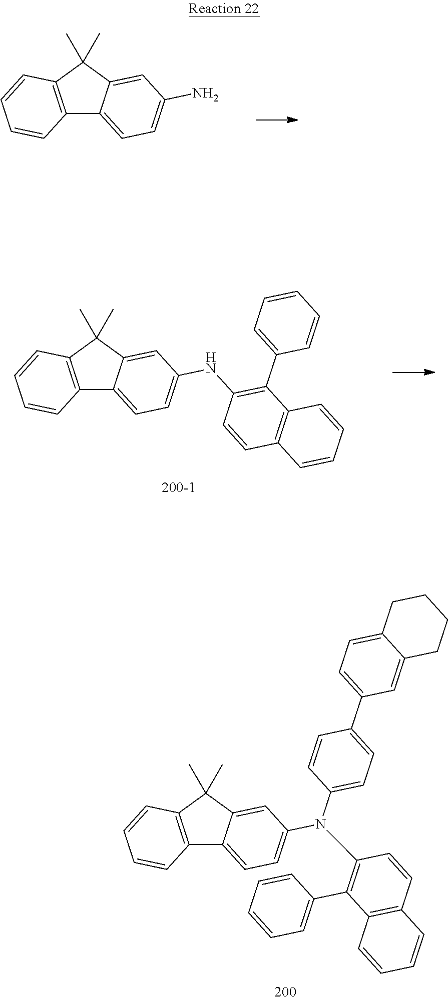 [SMILES: CC1(C)c2ccccc2-c2ccc(N)cc21 ; CC1(C)c2ccccc2-c2ccc(Nc3ccc4ccccc4c3-c3ccccc3)cc21 ; CC1(C)c2ccccc2-c2ccc(N(c3ccc(-c4ccc5c(c4)CCCC5)cc3)c3ccc4ccccc4c3-c3ccccc3)cc21]

2-amino-9,9-dimethylfluorene (22 mmol, 1.1 eq), Intermediate C5 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 200-1 was obtained (yield=50%).

Intermediate 200-1 (10 mmol, 1.1 eq), Intermediate C2 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 6.8 mmol of Compound 200 was obtained (yield=68%).

### (23) Synthesis of Amine Compound 205

Amine Compound 205 according to one or more embodiments may be synthesized by, for example, the steps of Reaction 23 below.

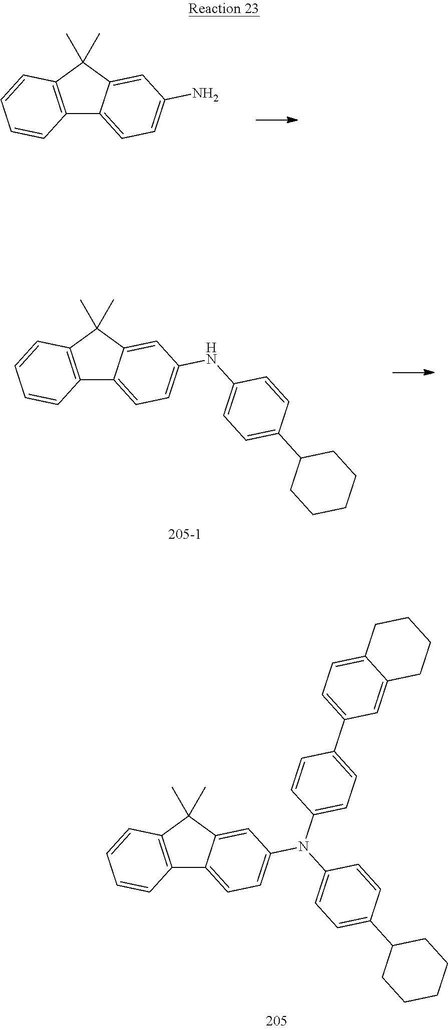 [SMILES: CC1(C)c2ccccc2-c2ccc(N)cc21 ; CC1(C)c2ccccc2-c2ccc(Nc3ccc(C4CCCCC4)cc3)cc21 ; CC1(C)c2ccccc2-c2ccc(N(c3ccc(-c4ccc5c(c4)CCCC5)cc3)c3ccc(C4CCCCC4)cc3)cc21]

2-amino-9,9-dimethylfluorene (22 mmol, 1.1 eq), Intermediate C6 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16 mmol of Intermediate 205-1 was obtained (yield=80%).

Intermediate 205-1 (16 mmol, 1.1 eq), Intermediate C2 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 10.15 mmol of Compound 205 was obtained (yield=70%).

### (24) Synthesis of Amine Compound 209

Amine Compound 209 according to one or more embodiments may be synthesized by, for example, the steps of Reaction 24 below.

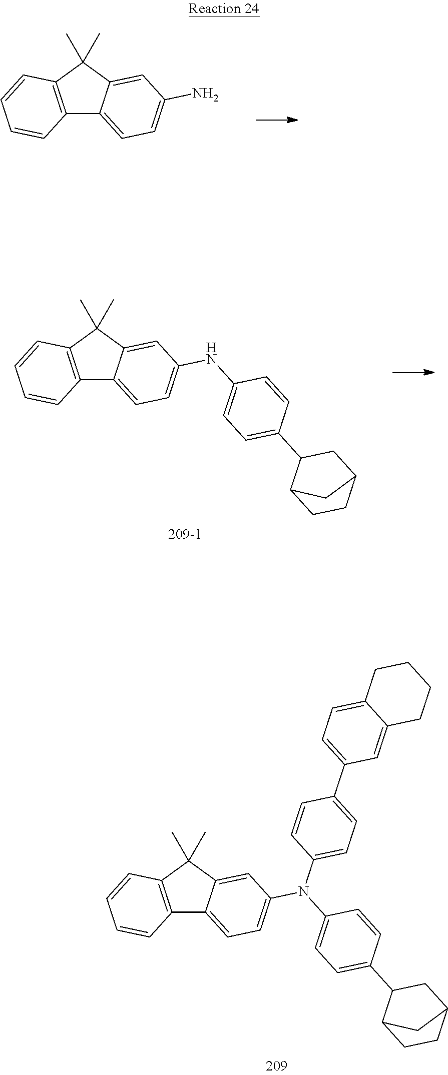 [SMILES: CC1(C)c2ccccc2-c2ccc(N)cc21 ; CC1(C)c2ccccc2-c2ccc(Nc3ccc(C4CC5CCC4C5)cc3)cc21 ; CC1(C)c2ccccc2-c2ccc(N(c3ccc(-c4ccc5c(c4)CCCC5)cc3)c3ccc(C4CC5CCC4C5)cc3)cc21]

2-amino-9,9-dimethylfluorene (22 mmol, 1.1 eq), Intermediate C7 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 16.8 mmol of Intermediate 209-1 was obtained (yield=84%).

Intermediate 209-1 (16 mmol, 1.1 eq), Intermediate C2 (14.5 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 10.85 mmol of Compound 209 was obtained (yield=75%).

### (25) Synthesis of Amine Compound 223

Amine Compound 223 according to one or more embodiments may be synthesized by, for example, the step of Reaction 25 below.

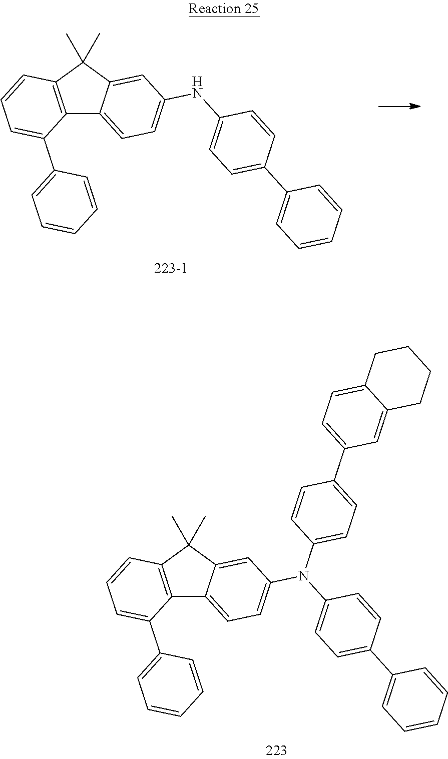 [SMILES: CC1(C)c2cc(Nc3ccc(-c4ccccc4)cc3)ccc2-c2c(-c3ccccc3)cccc21 ; CC1(C)c2cc(N(c3ccc(-c4ccccc4)cc3)c3ccc(-c4ccc5c(c4)CCCC5)cc3)ccc2-c2c(-c3ccccc3)cccc21]

Intermediate 223-1 (11 mmol, 1.1 eq), Intermediate C2 (10 mmol, 1 eq), Pd₂(dba)₃ (0.5 mol, 0.05 eq), t-BuONa (20 mmol, 2 eq), t-Bu₃P (1 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using MC/Hx=1/7. 7.6 mmol of Compound 223 was obtained (yield=76%).

### (26) Synthesis of Amine Compound 227

Amine Compound 227 according to one or more embodiments may be synthesized by, for example, the steps of Reaction 26 below.

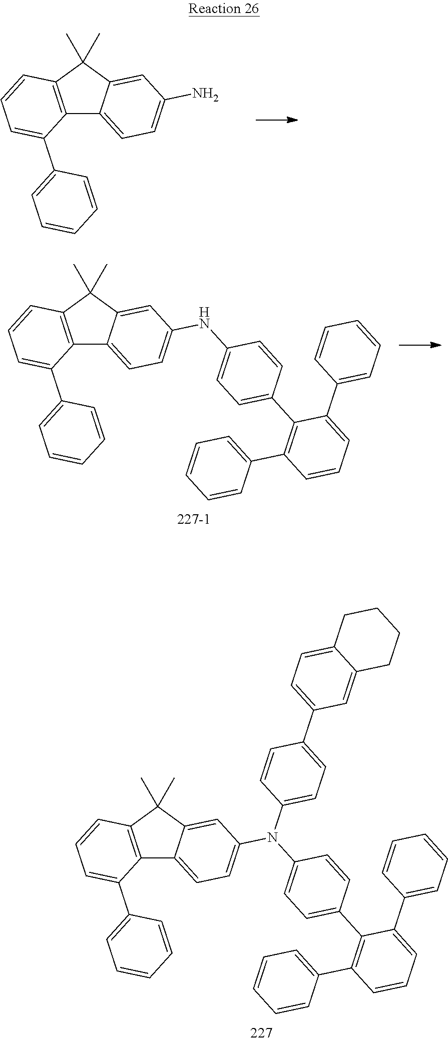

9,9-dimethyl-5-phenyl-9H-fluoren-2-amine (22 mmol, 1.1 eq), 4″-chloro-3′-phenyl-1,1′:2′,1″-terphenyl (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 227-1 was obtained (yield=50%).

Intermediate 227-1 (10 mmol, 1.1 eq), Intermediate C2 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 6.8 mmol of Compound 227 was obtained (yield=68%).

### (27) Synthesis of Amine Compound 239

Amine Compound 239 according to one or more embodiments may be synthesized by, for example, the steps of Reaction 27 below.

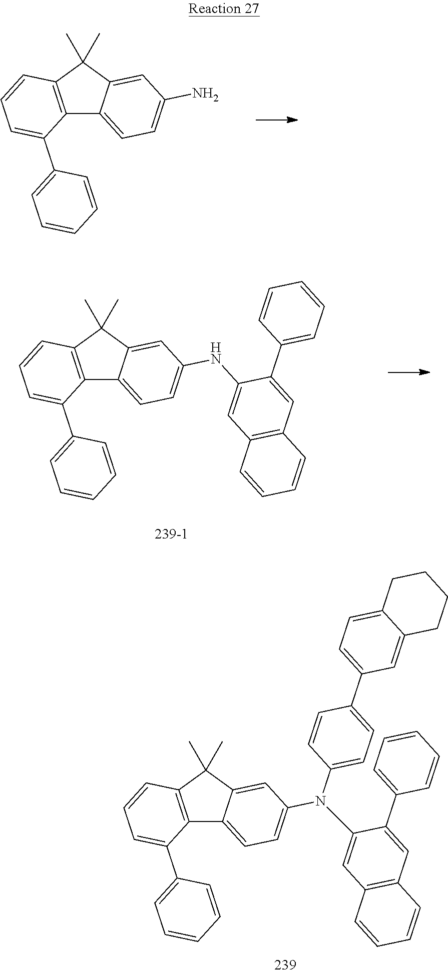 [SMILES: CC1(C)c2cc(N)ccc2-c2c(-c3ccccc3)cccc21 ; CC1(C)c2cc(Nc3cc4ccccc4cc3-c3ccccc3)ccc2-c2c(-c3ccccc3)cccc21 ; CC1(C)c2cc(N(c3ccc(-c4ccc5c(c4)CCCC5)cc3)c3cc4ccccc4cc3-c3ccccc3)ccc2-c2c(-c3ccccc3)cccc21]

9,9-dimethyl-5-phenyl-9H-fluoren-2-amine (22 mmol, 1.1 eq), Intermediate C4 (20 mmol, 1 eq), Pd₂(dba)₃ (1 mmol, 0.05 eq), t-BuONa (40 mmol, 2 eq), SPhos (2 mmol, 0.1 eq), and toluene (300 ml) were put in 1-neck-round flask and stirred at about 110° C. for about 1 hour. After finishing the reaction, the reaction product was worked-up using ether/H₂O, and separated by column chromatography using MC/Hx=1/5. 10 mmol of Intermediate 239-1 was obtained (yield=50%).

Intermediate 239-1 (10 mmol, 1.1 eq), Intermediate C2 (9 mmol, 1 eq), Pd₂(dba)₃ (0.7 mol, 0.05 eq), t-BuONa (29 mmol, 2 eq), t-Bu₃P (1.4 mmol, 0.1 eq), and toluene (150 ml) were put in 1-neck-round flask and stirred at about 110° C. for about 1 hour. After finishing the reaction, the reaction product was worked-up using ether/H₂O, and separated by column chromatography using Hx. 6.8 mmol of Compound 239 was obtained (yield=68%).

2. Manufacture and evaluation of light emitting element

### (1) Manufacture of Light Emitting Element

Light emitting elements including the amine compounds of embodiments or comparative compounds were manufactured by a method below. Light emitting elements of Example 1 to Example 27 were manufactured using Compounds 1, 2, 18, 19, 24, 28, 42, 43, 59, 60, 65, 68, 124, 125, 141, 142, 148, 151, 182, 183, 199, 200, 205, 209, 223, 227 and 239 as the materials of a hole transport layer. The light emitting element of Comparative Example 1 was manufactured using a material of NPB as the material of a hole transport layer. The light emitting elements of Comparative Examples 2 to 11 were manufactured using Comparative Compounds HX-1 to HX-10 as the materials of a hole transport layer.

In order to form an anode, an ITO glass substrate with about 15 Ω/cm² (thickness of 1200 Å) of Corning Co. was cut into a size of 50 mm×50 mm×0.7 mm, washed by ultrasonic waves using isopropyl alcohol and pure water for about 5 minutes each, and cleansed by exposing to ultraviolet rays for about 30 minutes and exposing to ozone, and this glass substrate was installed in a vacuum deposition apparatus.

On the substrate, a material of 2-TNATA was vacuum deposited to a thickness of about 600 Å as a hole injection layer, and the Example Compound or Comparative Compound was vacuum deposited to a thickness of about 300 Å as a hole transport layer. More particularly, in the light emitting elements of Examples 1 to 24 and Comparative Examples 1 to 11, the hole transport layer with a thickness of about 300 Å was formed as a single layer. In the light emitting elements of Examples 25 to 27, three hole transport layers (for example, first to third hole transport layers) were formed, and each of the three hole transport layers was formed into a thickness of about 100 Å. In the light emitting elements of Examples 25 to 27, the first hole transport layer and the third hole transport layer were formed using the Example Compounds, and the second hole transport layer was formed using Compound H-1-1.

On the hole transport layer, a blue fluorescence host of 9,10-di(naphthalene-2-yl)anthracene (hereinafter, DNA) and a blue fluorescence dopant of 4,4′-bis[2-(4-(N,N-diphenylamino)phenyl)vinyl]biphenyl (hereinafter, DPAVBi) were co-deposited in a weight ratio of about 98:2 to form an emission layer with a thickness of about 300 Å. On the emission layer, Alq₃ was deposited to a thickness of about 300 Å as an electron transport layer, and an alkaline metal halide of LiF was deposited on the electron transport layer to a thickness of about 10 Å as an electron injection layer. On the electron transport layer, Al was vacuum deposited to a thickness of about 3000 Å to form a LiF/Al electrode, to manufacture a light emitting element.

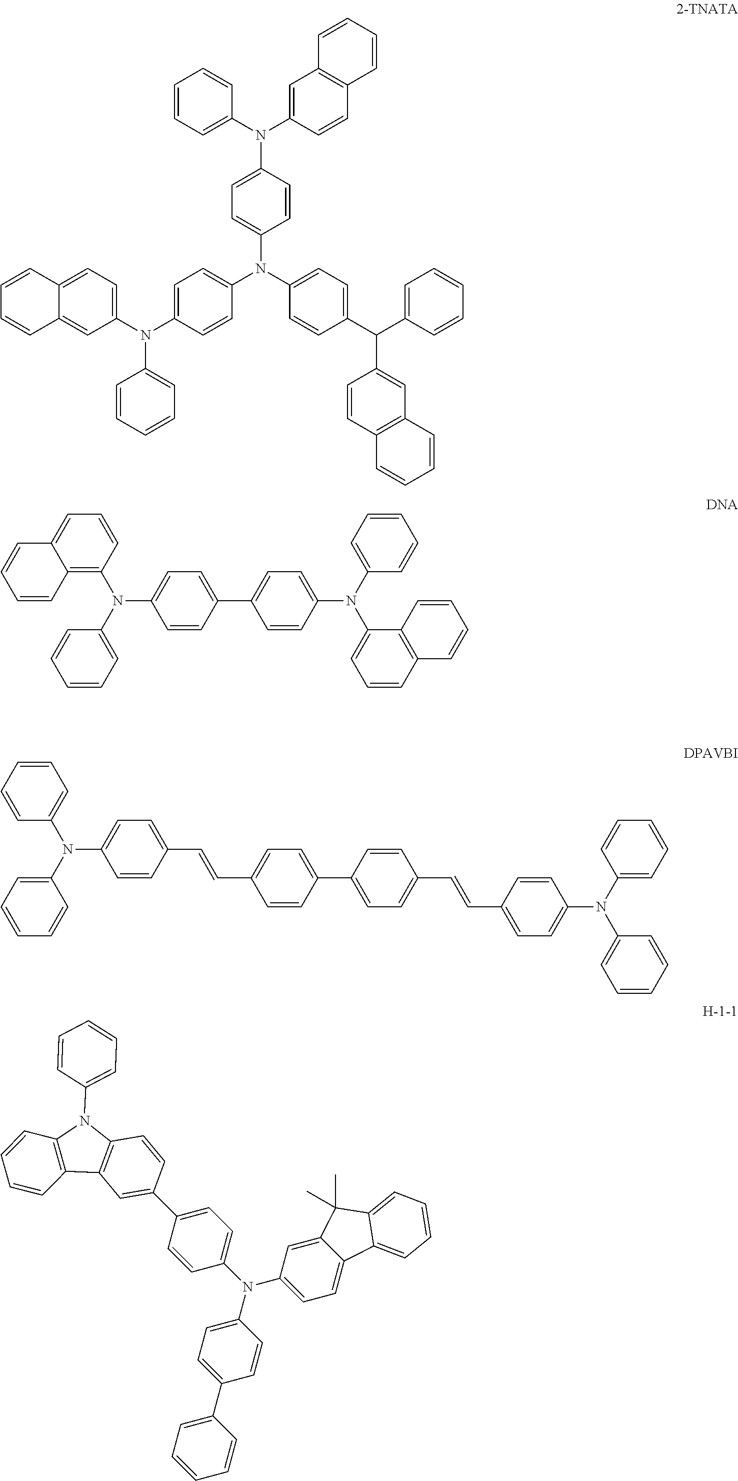

The Example Compounds and Comparative Compounds used in Examples 1 to 27 and Comparative Examples 2 to 11 are shown in Table 1.

TABLE 1

| | |
|---|---|
| Compound 1 | 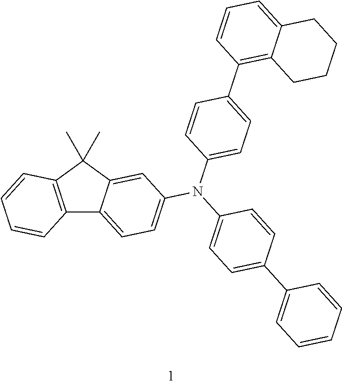 [SMILES: CC1(C)c2ccccc2-c2ccc(N(c3ccc(-c4ccccc4)cc3)c3ccc(-c4cccc5c4CCCC5)cc3)cc21] |
| Compound 2 | 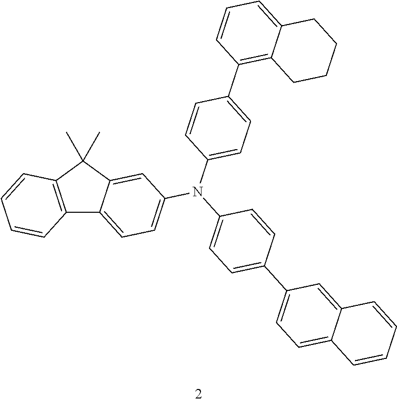 [SMILES: CC1(C)c2ccccc2-c2ccc(N(c3ccc(-c4ccc5ccccc5c4)cc3)c3ccc(-c4cccc5c4CCCC5)cc3)cc21] |
| Compound 18 | 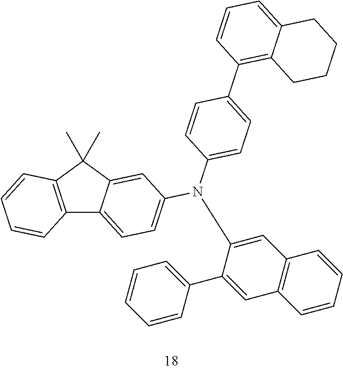 [SMILES: CC1(C)c2ccccc2-c2ccc(N(c3ccc(-c4cccc5c4CCCC5)cc3)c3cc4ccccc4cc3-c3ccccc3)cc21] |
| Compound 19 | 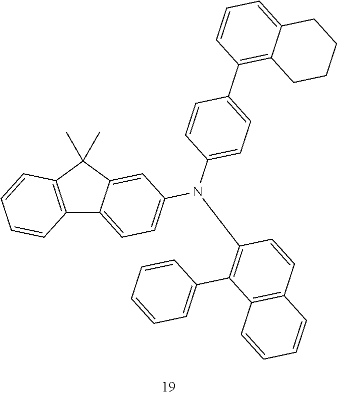 [SMILES: CC1(C)c2ccccc2-c2ccc(N(c3ccc(-c4cccc5c4CCCC5)cc3)c3ccc4ccccc4c3-c3ccccc3)cc21] |
| Compound 24 | 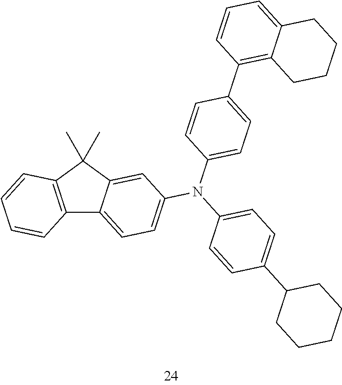 [SMILES: CC1(C)c2ccccc2-c2ccc(N(c3ccc(-c4cccc5c4CCCC5)cc3)c3ccc(C4CCCCC4)cc3)cc21] |
| Compound 28 | 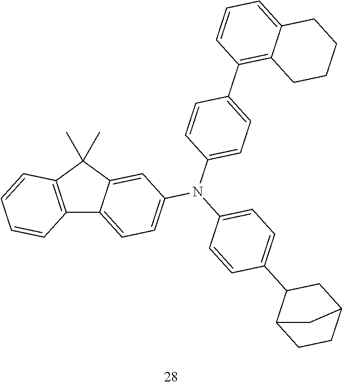 [SMILES: CC1(C)c2ccccc2-c2ccc(N(c3ccc(-c4cccc5c4CCCC5)cc3)c3ccc(C4CC5CCC4C5)cc3)cc21] |
| Compound 42 | 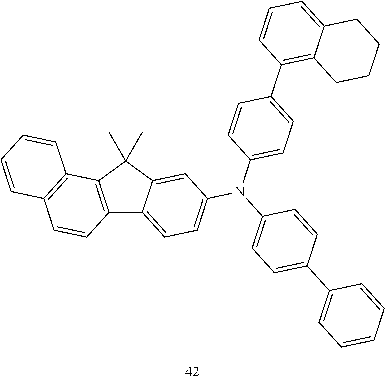 [SMILES: CC1(C)c2cc(N(c3ccc(-c4ccccc4)cc3)c3ccc(-c4cccc5c4CCCC5)cc3)ccc2-c2ccc3ccccc3c21] |
| Compound 43 | 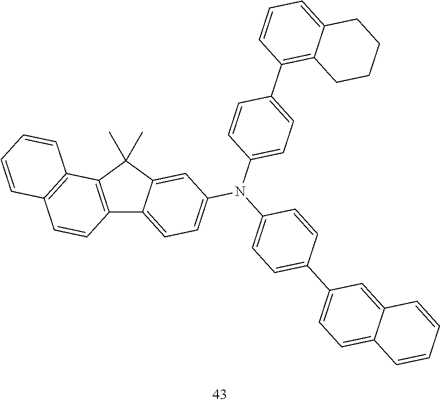 [SMILES: CC1(C)c2cc(N(c3ccc(-c4ccc5ccccc5c4)cc3)c3ccc(-c4cccc5c4CCCC5)cc3)ccc2-c2ccc3ccccc3c21] |
| Compound 59 | 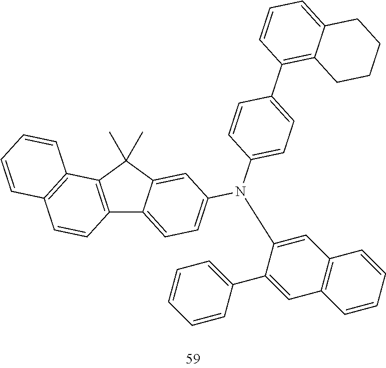 [SMILES: CC1(C)c2cc(N(c3ccc(-c4cccc5c4CCCC5)cc3)c3cc4ccccc4cc3-c3ccccc3)ccc2-c2ccc3ccccc3c21] |
| Compound 60 | 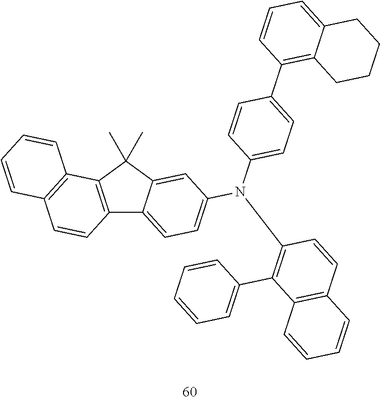 [SMILES: CC1(C)c2cc(N(c3ccc(-c4cccc5c4CCCC5)cc3)c3ccc4ccccc4c3-c3ccccc3)ccc2-c2ccc3ccccc3c21] |
| Compound 65 | 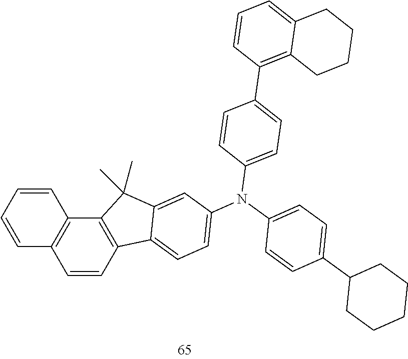 [SMILES: CC1(C)c2cc(N(c3ccc(-c4cccc5c4CCCC5)cc3)c3ccc(C4CCCCC4)cc3)ccc2-c2ccc3ccccc3c21] |
| Compound 68 | 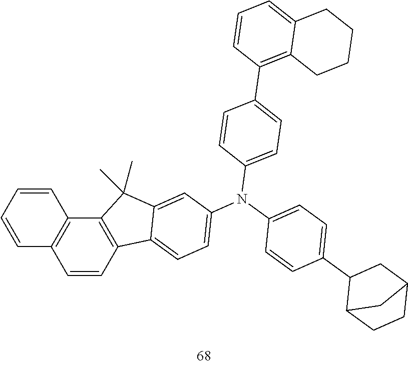 [SMILES: CC1(C)c2cc(N(c3ccc(-c4cccc5c4CCCC5)cc3)c3ccc(C4CC5CCC4C5)cc3)ccc2-c2ccc3ccccc3c21] |
| Compound 124 | 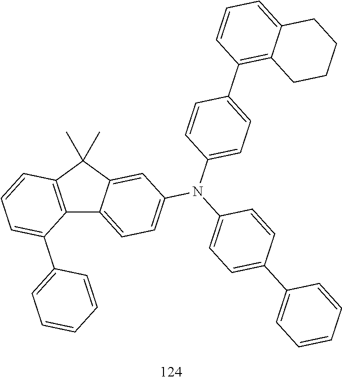 [SMILES: CC1(C)c2cc(N(c3ccc(-c4ccccc4)cc3)c3ccc(-c4cccc5c4CCCC5)cc3)ccc2-c2c(-c3ccccc3)cccc21] |
| Compound 125 | 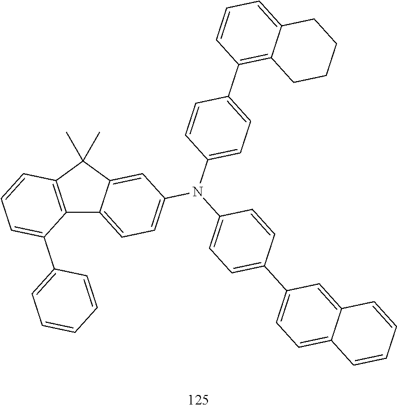 [SMILES: CC1(C)c2cc(N(c3ccc(-c4ccc5ccccc5c4)cc3)c3ccc(-c4cccc5c4CCCC5)cc3)ccc2-c2c(-c3ccccc3)cccc21] |
| Compound 141 | 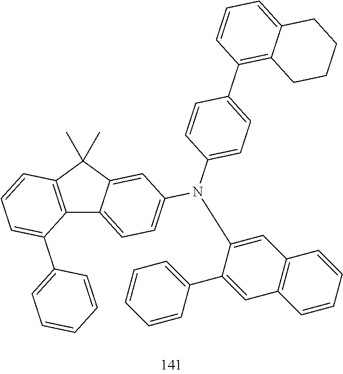 [SMILES: CC1(C)c2cc(N(c3ccc(-c4cccc5c4CCCC5)cc3)c3cc4ccccc4cc3-c3ccccc3)ccc2-c2c(-c3ccccc3)cccc21] |
| Compound 142 | 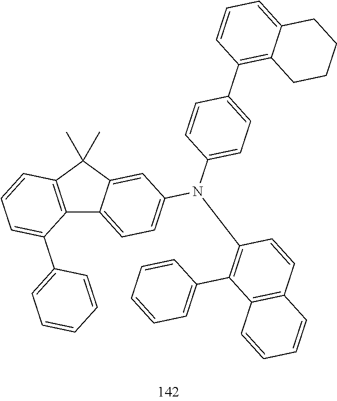 [SMILES: CC1(C)c2cc(N(c3ccc(-c4cccc5c4CCCC5)cc3)c3ccc4ccccc4c3-c3ccccc3)ccc2-c2c(-c3ccccc3)cccc21] |
| Compound 148 | 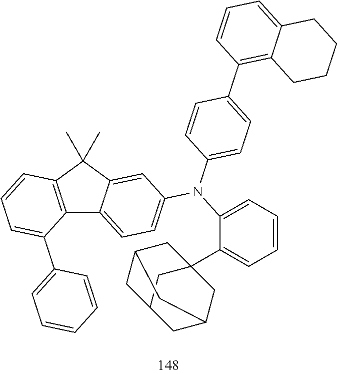 [SMILES: CC1(C)c2cc(N(c3ccc(-c4cccc5c4CCCC5)cc3)c3ccccc3C34CC5CC(CC(C5)C3)C4)ccc2-c2c(-c3ccccc3)cccc21] |
| Compound 151 | 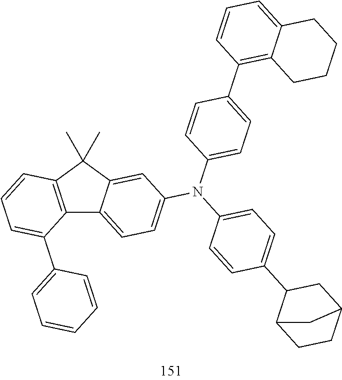 [SMILES: CC1(C)c2cc(N(c3ccc(-c4cccc5c4CCCC5)cc3)c3ccc(C4CC5CCC4C5)cc3)ccc2-c2c(-c3ccccc3)cccc21] |
| Compound 182 | 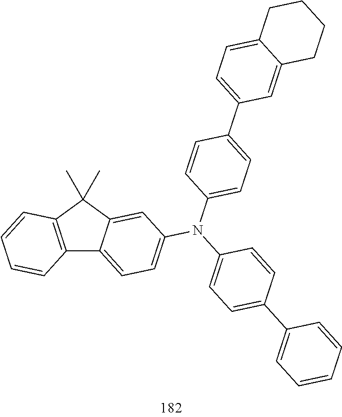 [SMILES: CC1(C)c2ccccc2-c2ccc(N(c3ccc(-c4ccccc4)cc3)c3ccc(-c4ccc5c(c4)CCCC5)cc3)cc21] |
| Compound 183 | 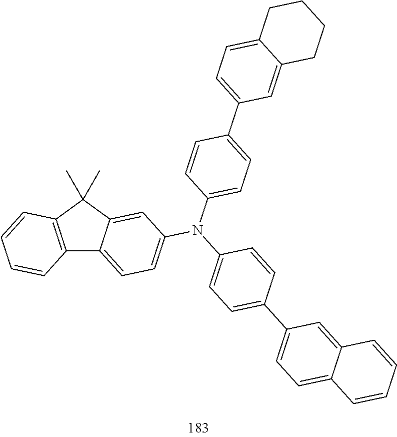 [SMILES: CC1(C)c2ccccc2-c2ccc(N(c3ccc(-c4ccc5c(c4)CCCC5)cc3)c3ccc(-c4ccc5ccccc5c4)cc3)cc21] |
| Compound 199 | 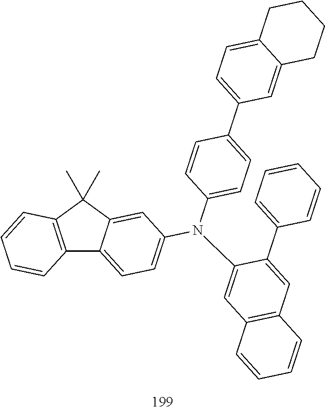 [SMILES: CC1(C)c2ccccc2-c2ccc(N(c3ccc(-c4ccc5c(c4)CCCC5)cc3)c3cc4ccccc4cc3-c3ccccc3)cc21] |
| Compound 200 | 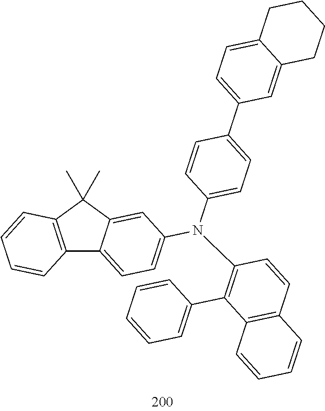 [SMILES: CC1(C)c2ccccc2-c2ccc(N(c3ccc(-c4ccc5c(c4)CCCC5)cc3)c3ccc4ccccc4c3-c3ccccc3)cc21] |
| Compound 205 | 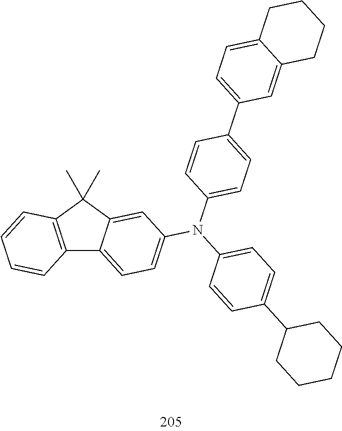 [SMILES: CC1(C)c2ccccc2-c2ccc(N(c3ccc(-c4ccc5c(c4)CCCC5)cc3)c3ccc(C4CCCCC4)cc3)cc21] |
| Compound 209 | 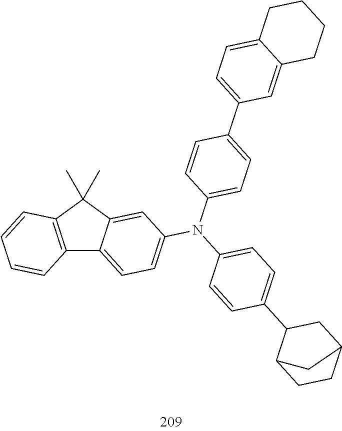 [SMILES: CC1(C)c2ccccc2-c2ccc(N(c3ccc(-c4ccc5c(c4)CCCC5)cc3)c3ccc(C4CC5CCC4C5)cc3)cc21] |
| Compound 223 | 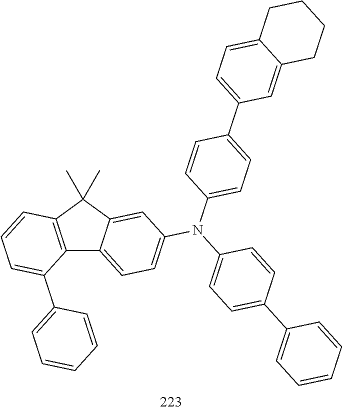 [SMILES: CC1(C)c2cc(N(c3ccc(-c4ccccc4)cc3)c3ccc(-c4ccc5c(c4)CCCC5)cc3)ccc2-c2c(-c3ccccc3)cccc21] |
| Compound 227 | 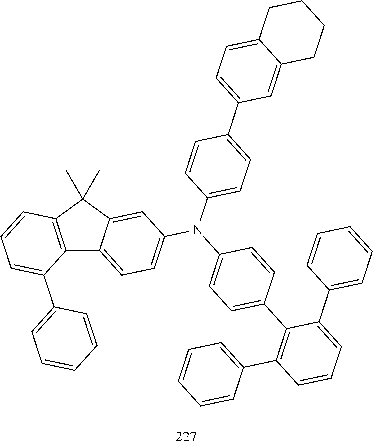 [SMILES: CC1(C)c2cc(N(c3ccc(-c4ccc5c(c4)CCCC5)cc3)c3ccc(-c4c(-c5ccccc5)cccc4-c4ccccc4)cc3)ccc2-c2c(-c3ccccc3)cccc21] |
| Compound 239 | 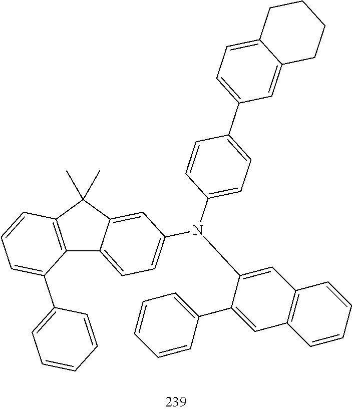 [SMILES: CC1(C)c2cc(N(c3ccc(-c4ccc5c(c4)CCCC5)cc3)c3cc4ccccc4cc3-c3ccccc3)ccc2-c2c(-c3ccccc3)cccc21] |
| Comparative Compound HX-1 | 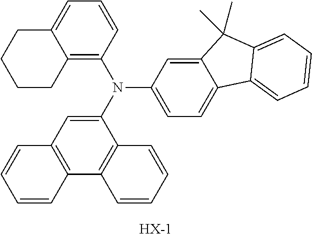 |
| Comparative Compound HX-2 | 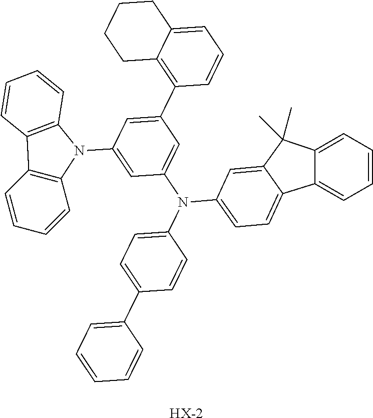 |
| Comparative Compound HX-3 | 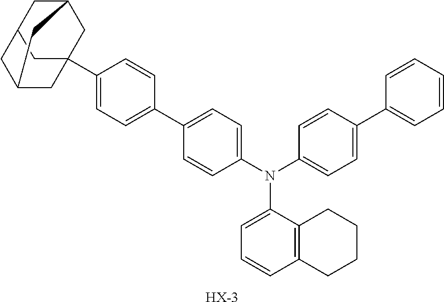 |
| Comparative Compound HX-4 | 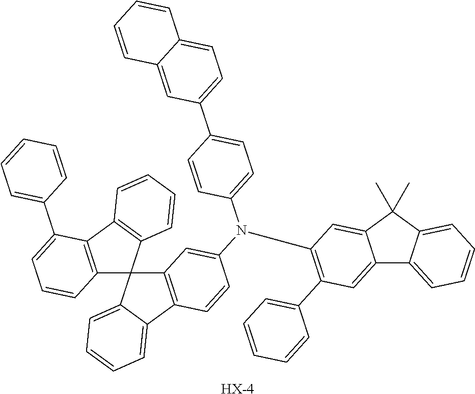 |
| Comparative Compound HX-5 | 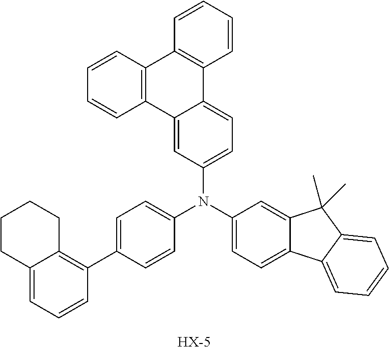 |
| Comparative Compound HX-6 | 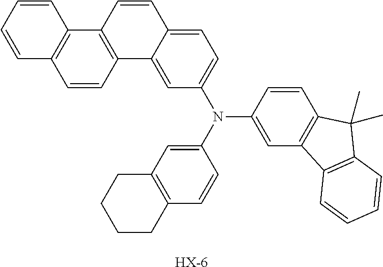 |
| Comparative Compound HX-7 | 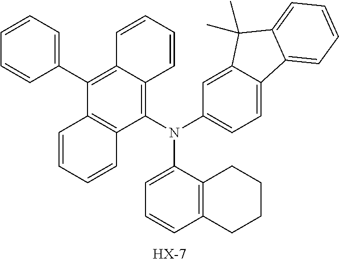 |
| Comparative Compound HX-8 | 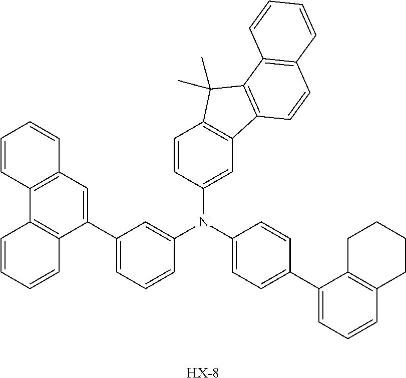 |
| Comparative Compound HX-9 | 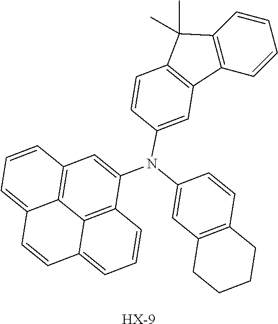 |
| Comparative Compound HX-10 | 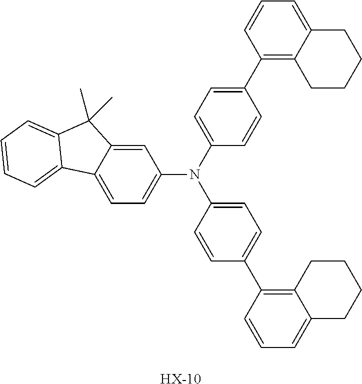 |

### (2) Evaluation of Properties of Light Emitting Elements

Table 2 shows evaluation results of the driving voltage, luminance, efficiency and half life of the light emitting elements of the Examples and Comparative Examples. As an evaluation apparatus, I-V-L Test System Polaronix V7000 (manufacturer: McSience Inc.) was used. The driving voltage, luminance and efficiency were evaluated based on a current density of about 50 mA/cm². The half life was evaluated based on a current density of about 100 mA/cm².

TABLE 2

| Element | | Driving | Current | | | | Half |
|---|---|---|---|---|---|---|---|
| manufacturing | | voltage | density | Luminance | Efficiency | Emission | life |
| example | Hole transport layer | (V) | (mA/cm²) | (cd/m²) | (cd/A) | color | (hr) |
| Comparative | NPB | 7.01 | 50 | 2645 | 5.29 | Blue | 258 |
| Example 1 | | | | | | | |
| Comparative | Comparative | 5.00 | 50 | 2350 | 4.70 | Blue | 400 |
| Example 2 | Compound HX-1 | | | | | | |
| Comparative | Comparative | 5.00 | 50 | 2150 | 4.30 | Blue | 380 |
| Example 3 | Compound HX-2 | | | | | | |
| Comparative | Comparative | 5.00 | 50 | 2700 | 5.40 | Blue | 410 |
| Example 4 | Compound HX-3 | | | | | | |
| Comparative | Comparative | 5.00 | 50 | 2750 | 5.50 | Blue | 420 |
| Example 5 | Compound HX-4 | | | | | | |
| Comparative | Comparative | 6.01 | 50 | 2645 | 5.29 | Blue | 350 |
| Example 6 | Compound HX-5 | | | | | | |
| Comparative | Comparative | 6.01 | 50 | 2150 | 4.30 | Blue | 320 |
| Example 7 | Compound HX-6 | | | | | | |
| Comparative | Comparative | 5.98 | 50 | 2750 | 5.50 | Blue | 345 |
| Example 8 | Compound HX-7 | | | | | | |
| Comparative | Comparative | 5.82 | 50 | 2750 | 5.50 | Blue | 365 |
| Example 9 | Compound HX-8 | | | | | | |
| Comparative | Comparative | 6.20 | 50 | 2645 | 5.29 | Blue | 360 |
| Example 10 | Compound HX-9 | | | | | | |
| Comparative | Comparative | 4.98 | 50 | 3240 | 6.48 | Blue | 420 |
| Example 11 | Compound HX-10 | | | | | | |
| Example 1 | Compound 1 | 4.78 | 50 | 3240 | 6.48 | Blue | 520 |
| Example 2 | Compound 2 | 4.81 | 50 | 3260 | 6.52 | Blue | 550 |
| Example 3 | Compound 18 | 4.71 | 50 | 3210 | 6.42 | Blue | 580 |
| Example 4 | Compound 19 | 4.75 | 50 | 3200 | 6.40 | Blue | 540 |
| Example 5 | Compound 24 | 4.92 | 50 | 3300 | 6.60 | Blue | 590 |
| Example 6 | Compound 28 | 4.72 | 50 | 3220 | 6.44 | Blue | 530 |
| Example 7 | Compound 42 | 4.75 | 50 | 3200 | 6.40 | Blue | 530 |
| Example 8 | Compound 43 | 4.85 | 50 | 3290 | 6.58 | Blue | 560 |
| Example 9 | Compound 59 | 4.81 | 50 | 3300 | 6.60 | Blue | 550 |
| Example 10 | Compound 60 | 4.79 | 50 | 3280 | 6.56 | Blue | 500 |
| Example 11 | Compound 65 | 4.78 | 50 | 3240 | 6.48 | Blue | 520 |
| Example 12 | Compound 68 | 4.71 | 50 | 3260 | 6.52 | Blue | 550 |
| Example 13 | Compound 124 | 4.71 | 50 | 3210 | 6.42 | Blue | 580 |
| Example 14 | Compound 125 | 4.75 | 50 | 3200 | 6.40 | Blue | 540 |
| Example 15 | Compound 141 | 4.82 | 50 | 3300 | 6.60 | Blue | 590 |
| Example 16 | Compound 142 | 4.72 | 50 | 3220 | 6.44 | Blue | 530 |
| Example 17 | Compound 148 | 4.75 | 50 | 3200 | 6.40 | Blue | 530 |
| Example 18 | Compound 151 | 4.85 | 50 | 3290 | 6.58 | Blue | 560 |
| Example 19 | Compound 182 | 4.71 | 50 | 3300 | 6.60 | Blue | 550 |
| Example 20 | Compound 183 | 4.79 | 50 | 3280 | 6.56 | Blue | 500 |
| Example 21 | Compound 199 | 4.75 | 50 | 3200 | 6.40 | Blue | 530 |
| Example 22 | Compound 200 | 4.84 | 50 | 3290 | 6.58 | Blue | 560 |
| Example 23 | Compound 205 | 4.71 | 50 | 3300 | 6.60 | Blue | 550 |
| Example 24 | Compound 209 | 4.79 | 50 | 3280 | 6.56 | Blue | 500 |
| Example 25 | Compound 223/Compound | 4.90 | 50 | 3360 | 6.72 | Blue | 680 |
| | H-1-1/Compound 223 | | | | | | |
| Example 26 | Compound 227/Compound | 4.90 | 50 | 3375 | 6.75 | Blue | 690 |
| | H-1-1/Compound 227 | | | | | | |
| Example 27 | Compound 239/Compound | 4.88 | 50 | 3375 | 6.75 | Blue | 690 |
| | H-1-1/Compound 239 | | | | | | |

Referring to Table 2, it could be found that the driving voltage was reduced, and the luminance was improved in the light emitting elements of Examples 1 to 27 when compared to the light emitting elements of Comparative Examples 1 to 5. In addition, it could be found that the efficiency was increased, and the lifetime was increased for the light emitting elements of Examples 1 to 27. The light emitting elements of Example 1 to 27 include Compounds 1, 2, 18, 19, 24, 28, 42, 43, 59, 60, 65, 68, 124, 125, 141, 142, 148, 151, 182, 183, 199, 200, 205, 209, 223, 227, and 239, and Compounds 1, 2, 18, 19, 24, 28, 42, 43, 59, 60, 65, 68, 124, 125, 141, 142, 148, 151, 182, 183, 199, 200, 205, 209, 223, 227, and 239 as the amine compounds of embodiments. The amine compound of one or more embodiments includes one fluorene moiety and one tetrahydronaphthyl group, and the fluorene moiety is directly bonded to the nitrogen atom of an amine group, and the tetrahydronaphthyl group is directly or indirectly bonded to the nitrogen atom of the amine group. Accordingly, the light emitting element, including the amine compound of one or more embodiments may show a low driving voltage, high luminance, high efficiency and long-life characteristics.

The light emitting element of Comparative Example 2 includes Comparative Compound HX-1, and Comparative Compound HX-1 includes a phenanthryl group. It could be found that the light emitting element including Comparative Compound HX-1 including a phenanthryl group showed an increased driving voltage, lower luminance and efficiency, and shorter lifetime when compared to the light emitting elements of Examples 1 to 27.

As described above, the amine compound of one or more embodiments does not include an anthracenyl group, a phenanthryl group, a pyrenyl group or a triphenylenyl group. When compared to a compound including an anthracenyl group, a phenanthryl group, a pyrenyl group and/or a triphenylenyl group, it could be found that the amine compound of one or more embodiments showed an improved difference between a HOMO energy level and a LUMO energy level, and showed improved hole transport properties. In addition, the light emitting element including the amine compound of one or more embodiments may show improved driving properties.

The light emitting element of Comparative Example 3 includes Comparative Compound HX-2, and Comparative Compound HX-2 includes a carbazole group. The amine compound of one or more embodiments does not include a carbazole group. It could be found that the light emitting element of Comparative Example 3, including Comparative Compound HX-2 including a carbazole group, showed an increased driving voltage, low luminance and efficiency, and short lifetime when compared to the light emitting elements of Examples 1 to 27.

The light emitting element of Comparative Example 4 includes Comparative Compound HX-3, and Comparative Compound HX-3 includes a tetrahydronaphthyl group but does not include a fluorene moiety. The amine compound of one or more embodiments and Comparative Compound HX-3 are different with respect to the inclusion of a fluorene moiety.

The light emitting element of Comparative Example 5 includes Comparative Compound HX-4, and Comparative Compound HX-4 does not include a tetrahydronaphthyl group and includes two fluorene moieties. The amine compound of one or more embodiments and Comparative Compound HX-4 are different with respect to the inclusion of the tetrahydronaphthyl group and the number of the fluorene moieties.

Comparative Compound HX-3 and Comparative Compound HX-4 do not include at least one of a tetrahydronaphthyl group and/or a fluorenyl group. It could be found that the light emitting elements of Comparative Example 4 and Comparative Example 5, including Comparative Compound HX-3 and Comparative Compound HX-4, not including at least one of the tetrahydronaphthyl group and/or the fluorenyl group, showed an increased driving voltage, low luminance and efficiency and short lifetime when compared to the light emitting elements of Examples 1 to 27.

The light emitting elements of Comparative Examples 6 to 10 include Comparative Compounds HX-5 to HX-9, and Comparative Compounds HX-5 to HX-9 include fused substituents of three or more phenyl groups (anthracenyl group, phenanthryl group, pyrenyl group, triphenylenyl group, chrysenyl group). Accordingly, it is believed that the light emitting elements of Comparative Examples 6 to 10, including Comparative Compounds HX-5 to HX-9, have a structure emitting light from a hole transport layer to inhibit the role of an emission layer. In addition, it is thought that the driving voltages are large, the efficiency is low, and the lifetime is short, because the energy levels of Comparative Compounds HX-5 to HX-9 are different from the emission zones. The emission zone means an area where holes and electrons are recombined.

The light emitting element of Comparative Example 11 includes Comparative Compound HX-10, and Comparative Compound HX-10 includes two hydronaphthyl groups. Comparative Compound HX-10 includes two aliphatic groups at the terminals thereof, and has a reduced refractive index, not smooth π-π conjugation, and low mobility. Accordingly, it is thought that the light emitting element of Comparative Example 11, including Comparative Compound HX-10, showed degraded hole mobility, the accumulation of electrons, and short lifetime.

The light emitting element of one or more embodiments may include a first electrode, a second electrode provided on the first electrode, and at least one functional layer provided between the first electrode and the second electrode. At least one functional layer may include the amine compound of one or more embodiments. The light emitting element including the amine compound of one or more embodiments may show a low driving voltage, high luminance, high efficiency and long-life characteristics.

The amine compound of one or more embodiments may include one fluorene moiety and one tetrahydronaphthyl group. Accordingly, the amine compound of one or more embodiments may show high or improved glass transition temperature and prevent or reduce crystallization. In addition, the amine compound of one or more embodiments may show low refractive index properties and excellent hole transport properties.

The light emitting element of one or more embodiments includes the amine compound of one or more embodiments and may show a low driving voltage, high luminance, high efficiency and long-life characteristics.

The amine compound of one or more embodiments may contribute to the decrease of the driving voltage, the improvement of the luminance and efficiency, and the increase of the lifetime of a light emitting element.

Although the embodiments of the present disclosure have been described, it is understood that the present disclosure should not be limited to these embodiments, but various changes and modifications can be made by one ordinary skilled in the art within the spirit and scope of the present disclosure as hereinafter claimed by the following claims and their equivalents.

What is claimed is:

1. A light emitting element, comprising:
a first electrode;
a second electrode facing the first electrode; and
at least one functional layer between the first electrode and the second electrode, the at least one functional layer comprising an amine compound represented by Formula 1: 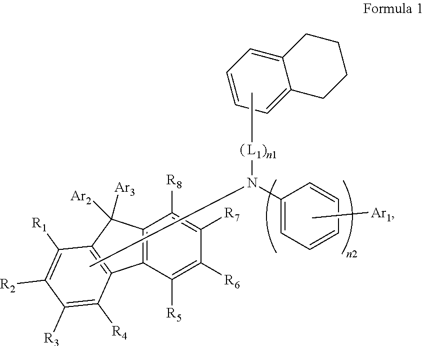
in Formula 1,
n1 is an integer of 0 to 2,
L₁ is a direct linkage, or an unsubstituted arylene group of 6 to 30 ring-forming carbon atoms,
n2 is 0 or 1,
Ar₁ is a substituted or unsubstituted methyl group, a substituted or unsubstituted cyclohexyl group, a substituted or unsubstituted norbornyl group, a substituted or unsubstituted adamantyl group, an unsubstituted phenyl group, an unsubstituted biphenyl group, a substituted or unsubstituted naphthyl group, or a substituted or unsubstituted terphenyl group,
Ar₂ and Ar₃ are each independently a substituted or unsubstituted silyl group, a substituted or unsubstituted alkoxy group of 1 to 30 carbon atoms, a substituted or unsubstituted alkyl group of 1 to 30 carbon atoms, a substituted or unsubstituted alkenyl group of 2 to 30 carbon atoms, a substituted or unsubstituted alkynyl group of 2 to 30 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and
R₁ to R₈ are each independently a hydrogen atom, a deuterium atom, a cyano group, a nitro group, a hydroxyl group, a halogen atom, a substituted or unsubstituted silyl group, a substituted or unsubstituted alkoxy group of 1 to 30 carbon atoms, a substituted or unsubstituted alkyl group of 1 to 30 carbon atoms, a substituted or unsubstituted alkenyl group of 2 to 30 carbon atoms, a substituted or unsubstituted alkynyl group or 2 to 30 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

2. The light emitting element of claim 1, wherein, in Formula 1, Ar₁ is represented by any one of Formulae A1-1 to A1-8: 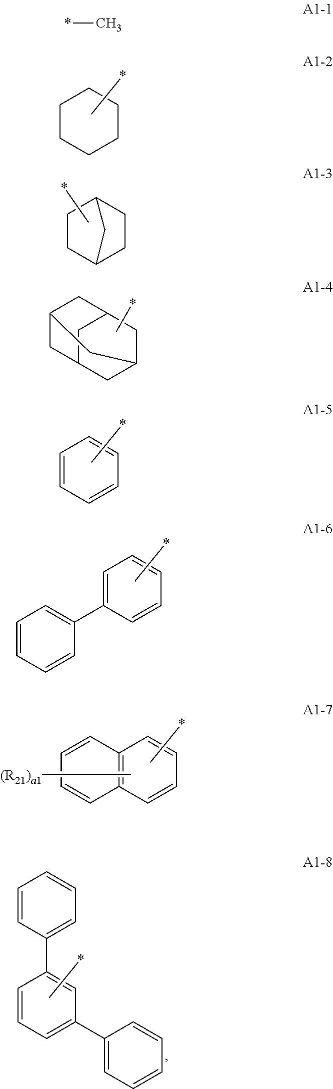
in Formula A1-7,
a1 is an integer of 0 to 7, and
R₂₁ is a hydrogen atom, a deuterium atom, or a substituted or unsubstituted aryl group of 6 to 30 ring-forming carbon atoms.

3. The light emitting element of claim 1, wherein Formula 1 is represented by any one of Formula 1-1A to Formula 1-1C: 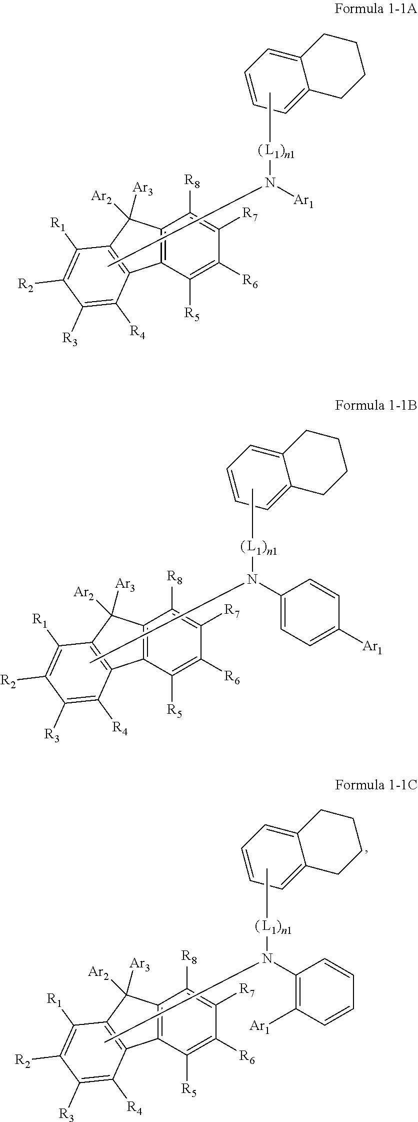
in Formula 1-1A to Formula 1-1C,
Ar₁ to Ar₃, L₁, n1, and R₁ to R₈ are the same as defined in Formula 1.

4. The light emitting element of claim 1, wherein Formula 1 is represented by any one of Formula 1-2A to Formula 1-2D: 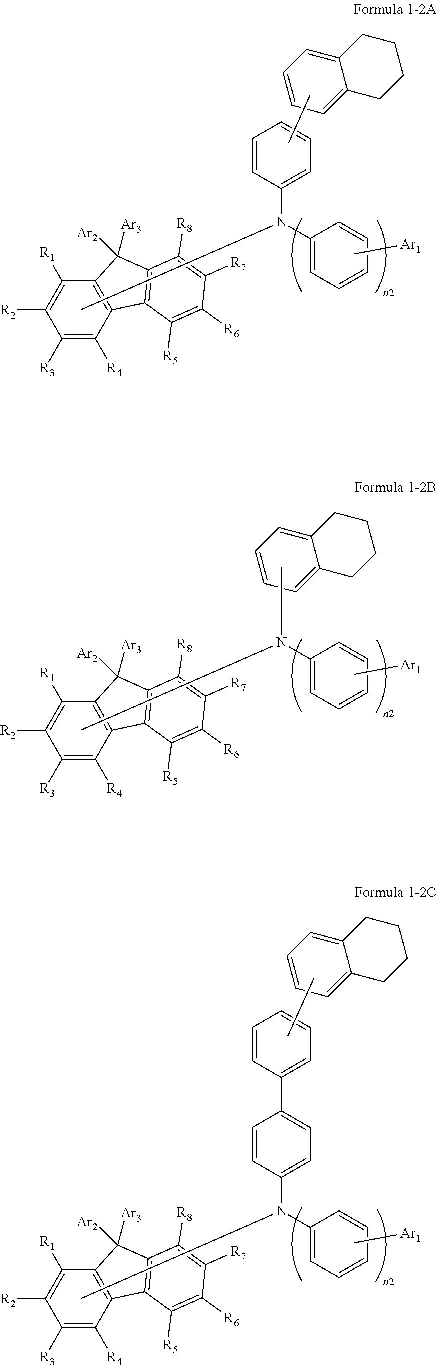 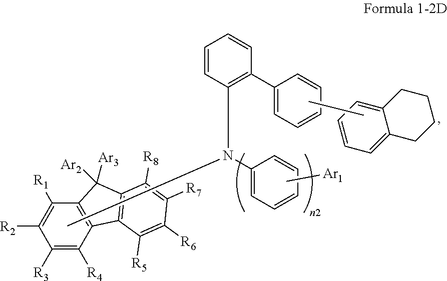
in Formula 1-2A to Formula 1-2D,
Ar₁ to Ar₃, n2, and R₁ to R₈ are the same as defined in Formula 1.

5. The light emitting element of claim 4, wherein Formula 1-2A is represented by any one of Formula 1-2AA or Formula 1-2AB: 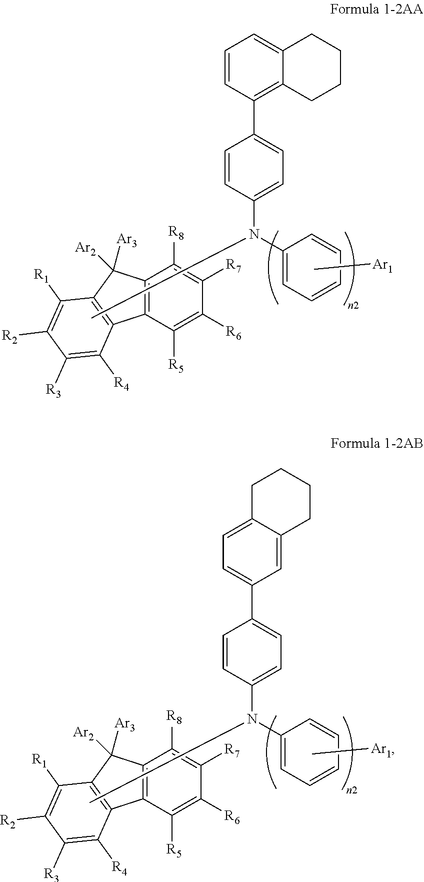
in Formula 1-2AA and Formula 1-2AB,
Ar₁ to Ar₃, n2, and R₁ to R₈ are the same as defined in Formula 1-2A.

6. The light emitting element of claim 1, wherein Formula 1 is represented by any one of Formula 1-3A to Formula 1-3C: 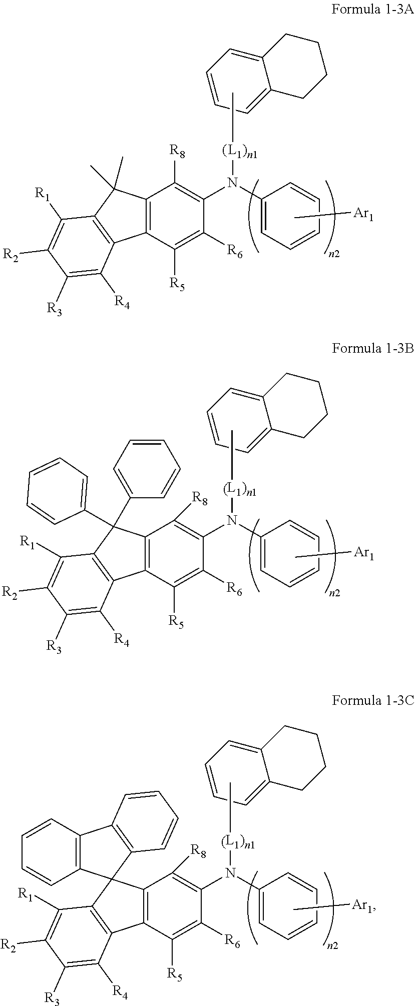
in Formula 1-3A to Formula 1-3C,
Ar₁, L₁, n1, n2, and R₁ to R₈ are the same as defined in Formula 1.

7. The light emitting element of claim 6, wherein Formula 1-3A is represented by any one of Formula 1-3AA to Formula 1-3AC: 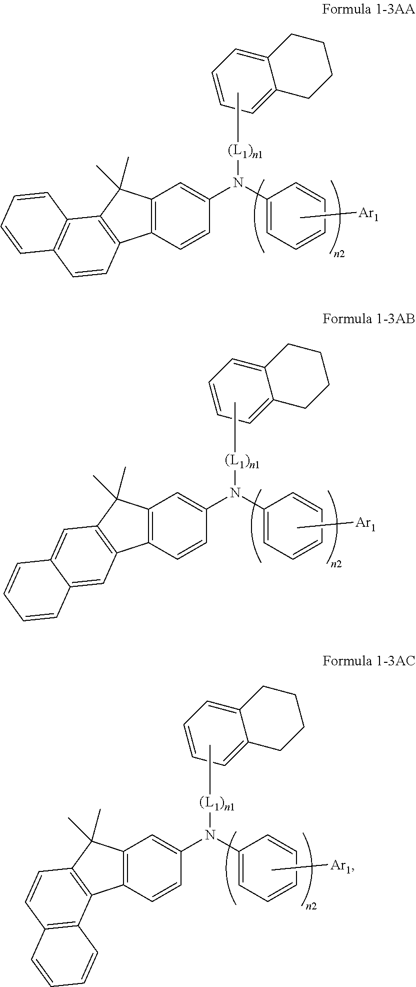
in Formula 1-3AA to Formula 1-3AC,
Ar₁, L₁, n1, and n2 are the same as defined in Formula 1-3A.

8. The light emitting element of claim 6, wherein Formula 1-3A is represented by Formula 1-3AD: 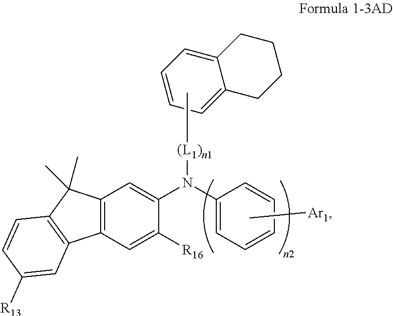
in Formula 1-3AD,
R₁₃ and R₁₆ are each independently a hydrogen atom, a substituted or unsubstituted methyl group, or a substituted or unsubstituted phenyl group, and
Ar₁, L₁, n1 and n2 are the same as defined in Formula 1-3A.

9. The light emitting element of claim 1, wherein the at least one functional layer comprises an emission layer, a hole transport region between the first electrode and the emission layer, and an electron transport region between the emission layer and the second electrode, and
the hole transport region comprises the amine compound.

10. The light emitting element of claim 9, wherein the hole transport region comprises a hole injection layer on the first electrode, a hole transport layer on the hole injection layer, and an electron blocking layer on the hole transport layer, and
at least one of the hole injection layer, the hole transport layer, or the electron blocking layer comprises the amine compound.

11. The light emitting element of claim 1, wherein the amine compound is represented by any one of compounds in Compound Group 1:

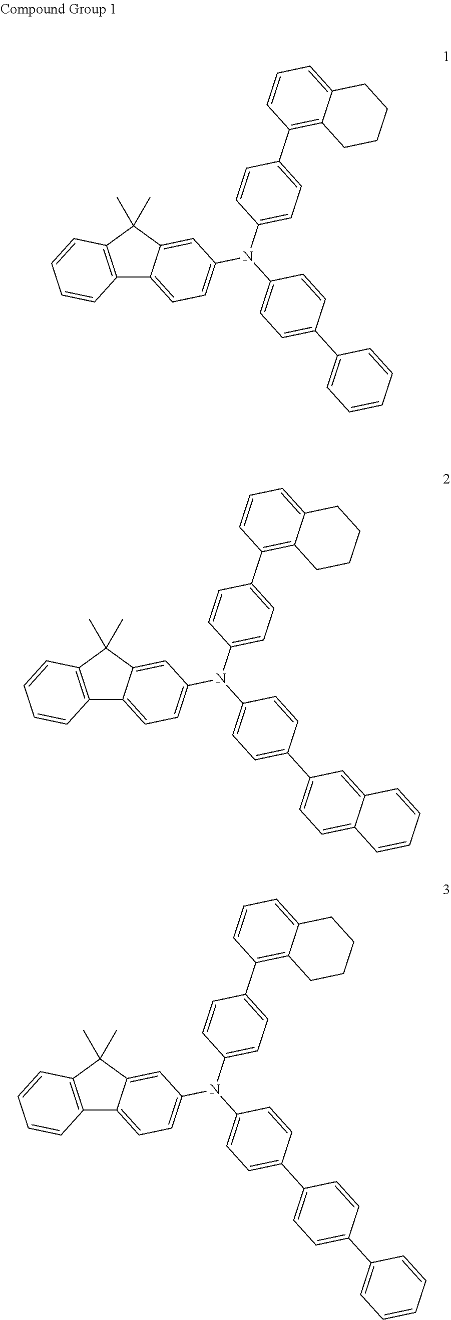 [SMILES: CC1(C)c2ccccc2-c2ccc(N(c3ccc(-c4ccccc4)cc3)c3ccc(-c4cccc5c4CCCC5)cc3)cc21 ; CC1(C)c2ccccc2-c2ccc(N(c3ccc(-c4ccc5ccccc5c4)cc3)c3ccc(-c4cccc5c4CCCC5)cc3)cc21 ; CC1(C)c2ccccc2-c2ccc(N(c3ccc(-c4ccc(-c5ccccc5)cc4)cc3)c3ccc(-c4cccc5c4CCCC5)cc3)cc21]

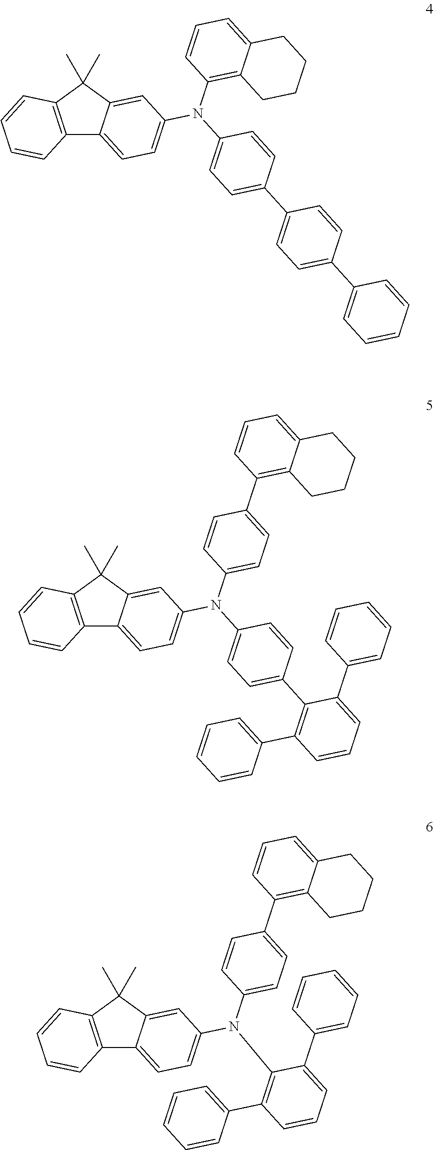 [SMILES: CC1(C)c2ccccc2-c2ccc(N(c3ccc(-c4ccc(-c5ccccc5)cc4)cc3)c3cccc4c3CCCC4)cc21 ; CC1(C)c2ccccc2-c2ccc(N(c3ccc(-c4cccc5c4CCCC5)cc3)c3ccc(-c4c(-c5ccccc5)cccc4-c4ccccc4)cc3)cc21 ; CC1(C)c2ccccc2-c2ccc(N(c3ccc(-c4cccc5c4CCCC5)cc3)c3c(-c4ccccc4)cccc3-c3ccccc3)cc21]

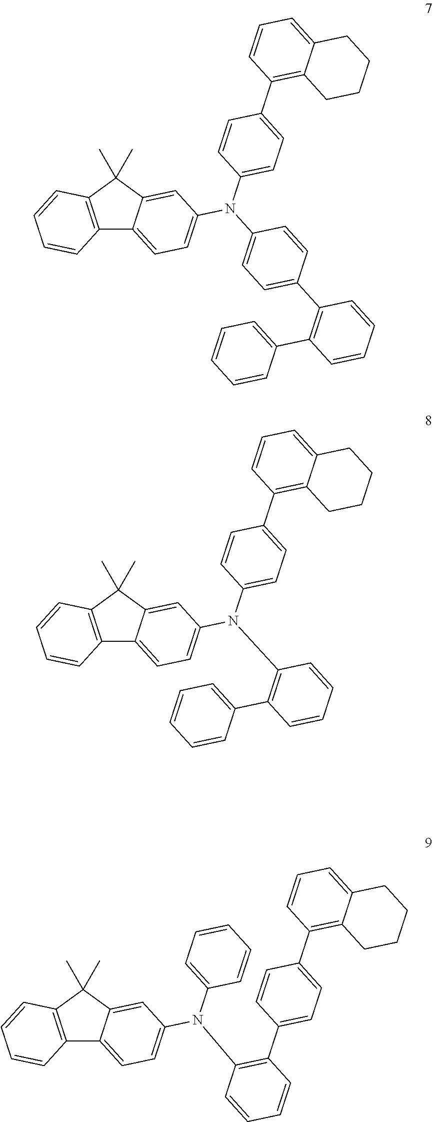 [SMILES: CC1(C)c2ccccc2-c2ccc(N(c3ccc(-c4ccccc4-c4ccccc4)cc3)c3ccc(-c4cccc5c4CCCC5)cc3)cc21 ; CC1(C)c2ccccc2-c2ccc(N(c3ccc(-c4cccc5c4CCCC5)cc3)c3ccccc3-c3ccccc3)cc21 ; CC1(C)c2ccccc2-c2ccc(N(c3ccccc3)c3ccccc3-c3ccc(-c4cccc5c4CCCC5)cc3)cc21]

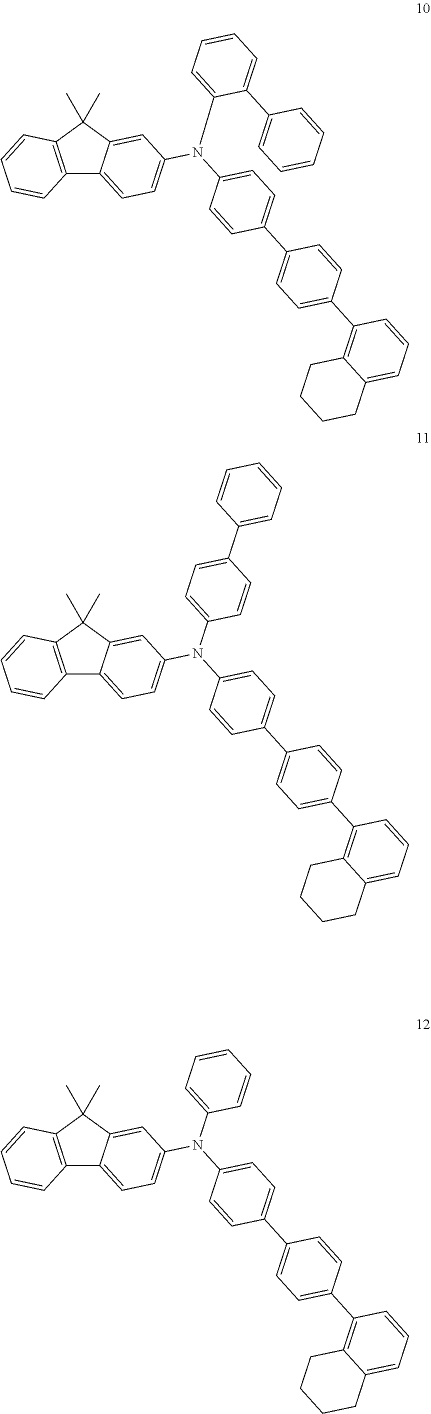 [SMILES: CC1(C)c2ccccc2-c2ccc(N(c3ccc(-c4ccc(-c5cccc6c5CCCC6)cc4)cc3)c3ccccc3-c3ccccc3)cc21 ; CC1(C)c2ccccc2-c2ccc(N(c3ccc(-c4ccccc4)cc3)c3ccc(-c4ccc(-c5cccc6c5CCCC6)cc4)cc3)cc21 ; CC1(C)c2ccccc2-c2ccc(N(c3ccccc3)c3ccc(-c4ccc(-c5cccc6c5CCCC6)cc4)cc3)cc21]

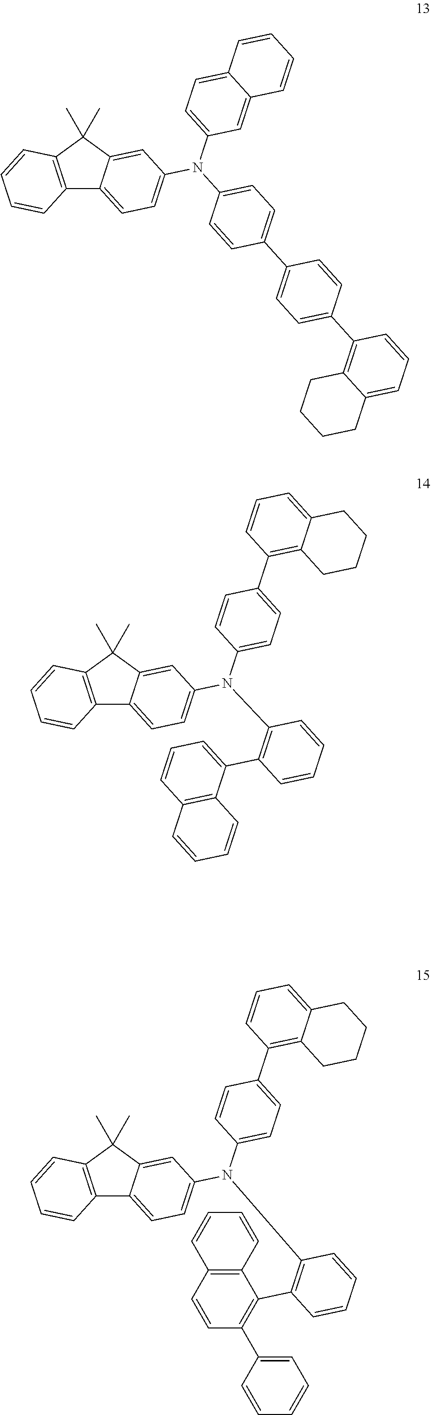 [SMILES: CC1(C)c2ccccc2-c2ccc(N(c3ccc(-c4ccc(-c5cccc6c5CCCC6)cc4)cc3)c3ccc4ccccc4c3)cc21 ; CC1(C)c2ccccc2-c2ccc(N(c3ccc(-c4cccc5c4CCCC5)cc3)c3ccccc3-c3cccc4ccccc34)cc21 ; CC1(C)c2ccccc2-c2ccc(N(c3ccc(-c4cccc5c4CCCC5)cc3)c3ccccc3-c3c(-c4ccccc4)ccc4ccccc34)cc21]

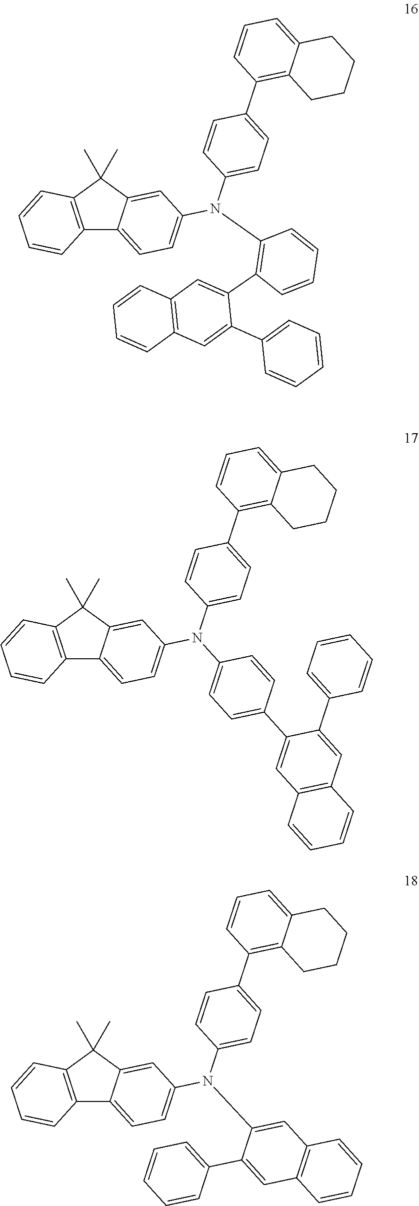 [SMILES: CC1(C)c2ccccc2-c2ccc(N(c3ccc(-c4cccc5c4CCCC5)cc3)c3ccccc3-c3cc4ccccc4cc3-c3ccccc3)cc21 ; CC1(C)c2ccccc2-c2ccc(N(c3ccc(-c4cc5ccccc5cc4-c4ccccc4)cc3)c3ccc(-c4cccc5c4CCCC5)cc3)cc21 ; CC1(C)c2ccccc2-c2ccc(N(c3ccc(-c4cccc5c4CCCC5)cc3)c3cc4ccccc4cc3-c3ccccc3)cc21]

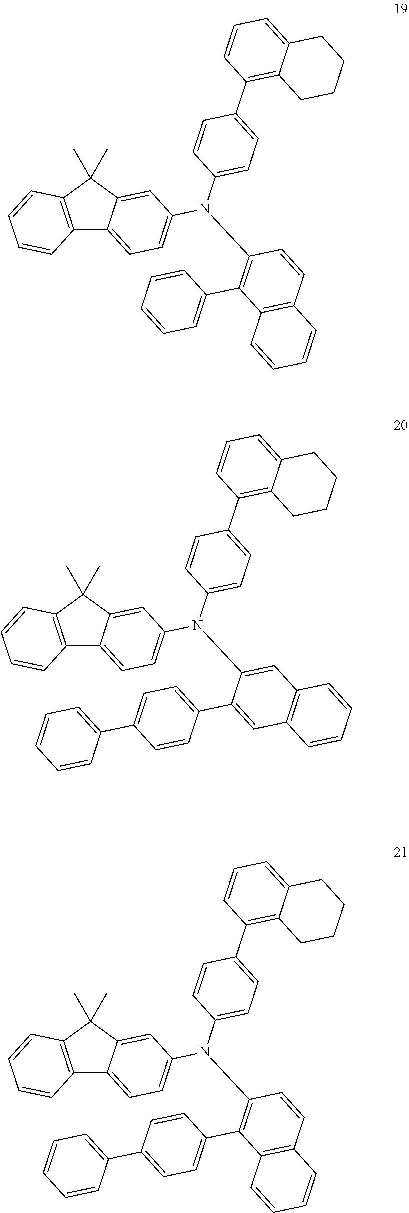 [SMILES: CC1(C)c2ccccc2-c2ccc(N(c3ccc(-c4cccc5c4CCCC5)cc3)c3ccc4ccccc4c3-c3ccccc3)cc21 ; CC1(C)c2ccccc2-c2ccc(N(c3ccc(-c4cccc5c4CCCC5)cc3)c3cc4ccccc4cc3-c3ccc(-c4ccccc4)cc3)cc21 ; CC1(C)c2ccccc2-c2ccc(N(c3ccc(-c4cccc5c4CCCC5)cc3)c3ccc4ccccc4c3-c3ccc(-c4ccccc4)cc3)cc21]

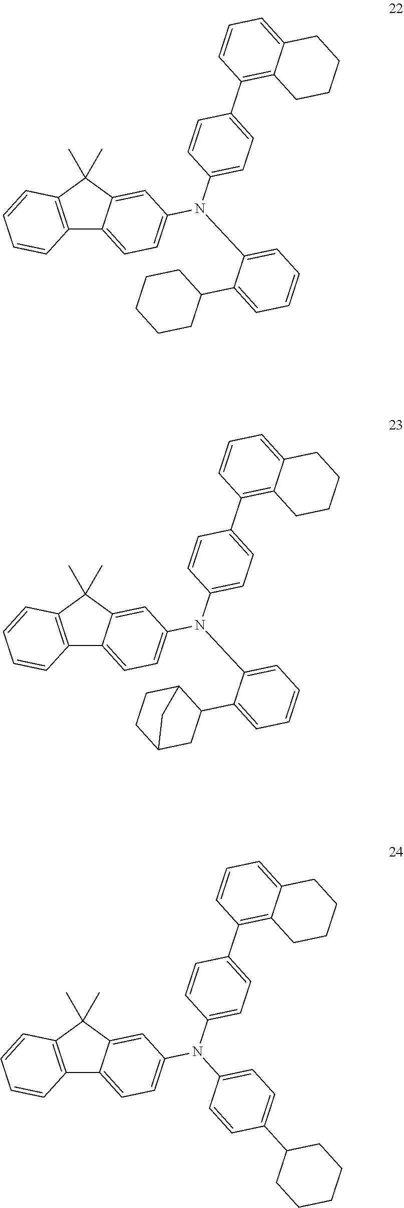 [SMILES: CC1(C)c2ccccc2-c2ccc(N(c3ccc(-c4cccc5c4CCCC5)cc3)c3ccccc3C3CCCCC3)cc21 ; CC1(C)c2ccccc2-c2ccc(N(c3ccc(-c4cccc5c4CCCC5)cc3)c3ccccc3C3CC4CCC3C4)cc21 ; CC1(C)c2ccccc2-c2ccc(N(c3ccc(-c4cccc5c4CCCC5)cc3)c3ccc(C4CCCCC4)cc3)cc21]

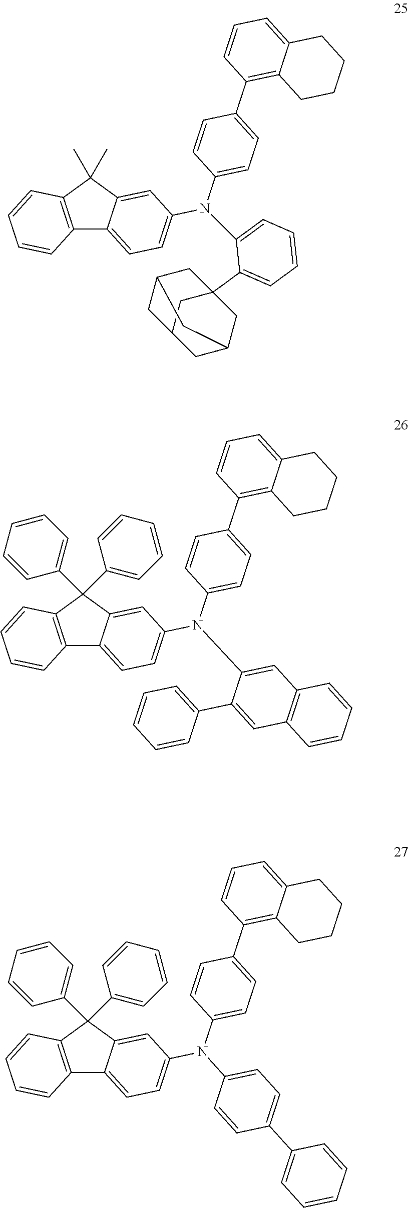 [SMILES: CC1(C)c2ccccc2-c2ccc(N(c3ccc(-c4cccc5c4CCCC5)cc3)c3ccccc3C34CC5CC(CC(C5)C3)C4)cc21 ; c1ccc(-c2cc3ccccc3cc2N(c2ccc(-c3cccc4c3CCCC4)cc2)c2ccc3c(c2)C(c2ccccc2)(c2ccccc2)c2ccccc2-3)cc1 ; c1ccc(-c2ccc(N(c3ccc(-c4cccc5c4CCCC5)cc3)c3ccc4c(c3)C(c3ccccc3)(c3ccccc3)c3ccccc3-4)cc2)cc1]

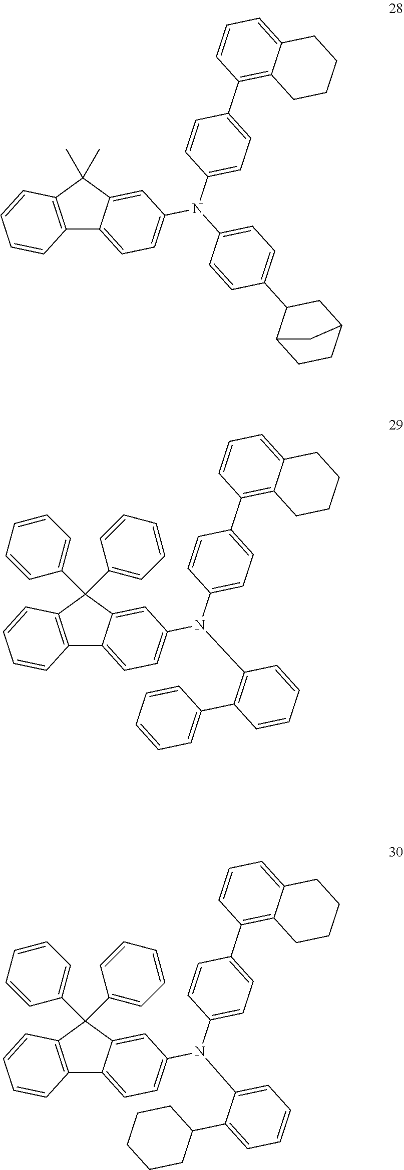 [SMILES: CC1(C)c2ccccc2-c2ccc(N(c3ccc(-c4cccc5c4CCCC5)cc3)c3ccc(C4CC5CCC4C5)cc3)cc21 ; c1ccc(-c2ccccc2N(c2ccc(-c3cccc4c3CCCC4)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5cccc6c5CCCC6)cc4)c4ccccc4C4CCCCC4)cc32)cc1]

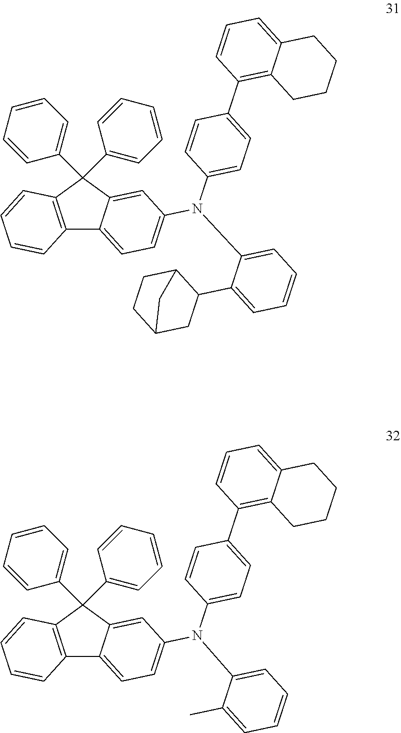 [SMILES: c1ccc(C2(c3ccccc3)c3ccccc3-c3ccc(N(c4ccc(-c5cccc6c5CCCC6)cc4)c4ccccc4C4CC5CCC4C5)cc32)cc1 ; Cc1ccccc1N(c1ccc(-c2cccc3c2CCCC3)cc1)c1ccc2c(c1)C(c1ccccc1)(c1ccccc1)c1ccccc1-2]

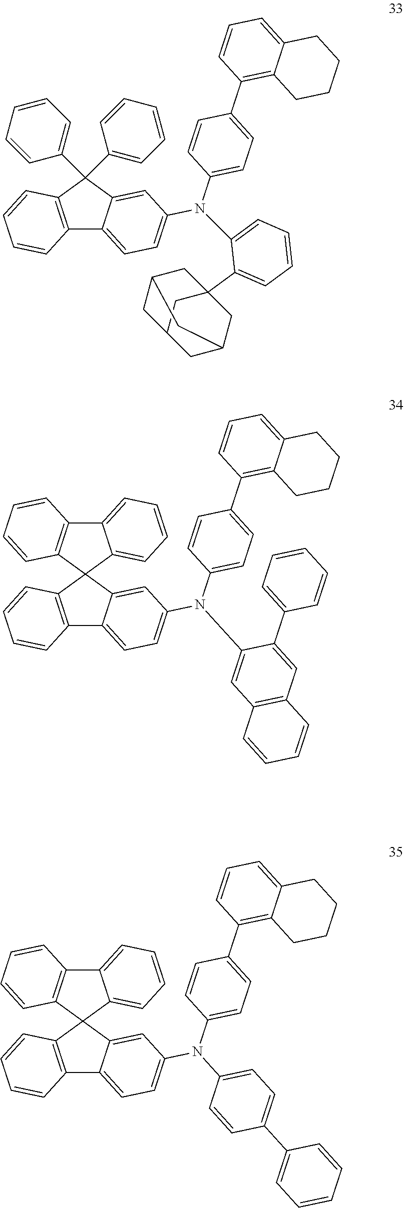 [SMILES: c1ccc(C2(c3ccccc3)c3ccccc3-c3ccc(N(c4ccc(-c5cccc6c5CCCC6)cc4)c4ccccc4C45CC6CC(CC(C6)C4)C5)cc32)cc1 ; c1ccc(-c2cc3ccccc3cc2N(c2ccc(-c3cccc4c3CCCC4)cc2)c2ccc3c(c2)C2(c4ccccc4-c4ccccc42)c2ccccc2-3)cc1 ; c1ccc(-c2ccc(N(c3ccc(-c4cccc5c4CCCC5)cc3)c3ccc4c(c3)C3(c5ccccc5-c5ccccc53)c3ccccc3-4)cc2)cc1]

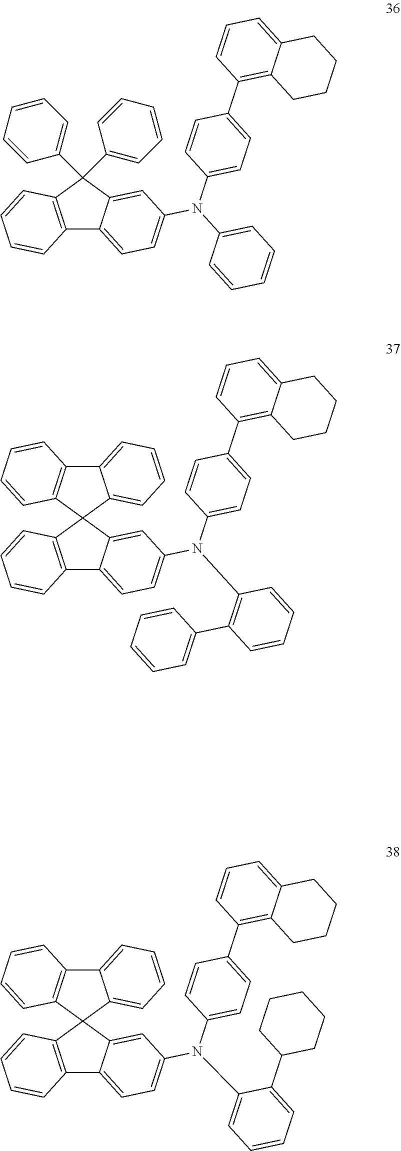 [SMILES: c1ccc(N(c2ccc(-c3cccc4c3CCCC4)cc2)c2ccc3c(c2)C(c2ccccc2)(c2ccccc2)c2ccccc2-3)cc1 ; c1ccc(-c2ccccc2N(c2ccc(-c3cccc4c3CCCC4)cc2)c2ccc3c(c2)C2(c4ccccc4-c4ccccc42)c2ccccc2-3)cc1 ; c1ccc2c(c1)-c1ccccc1C21c2ccccc2-c2ccc(N(c3ccc(-c4cccc5c4CCCC5)cc3)c3ccccc3C3CCCCC3)cc21]

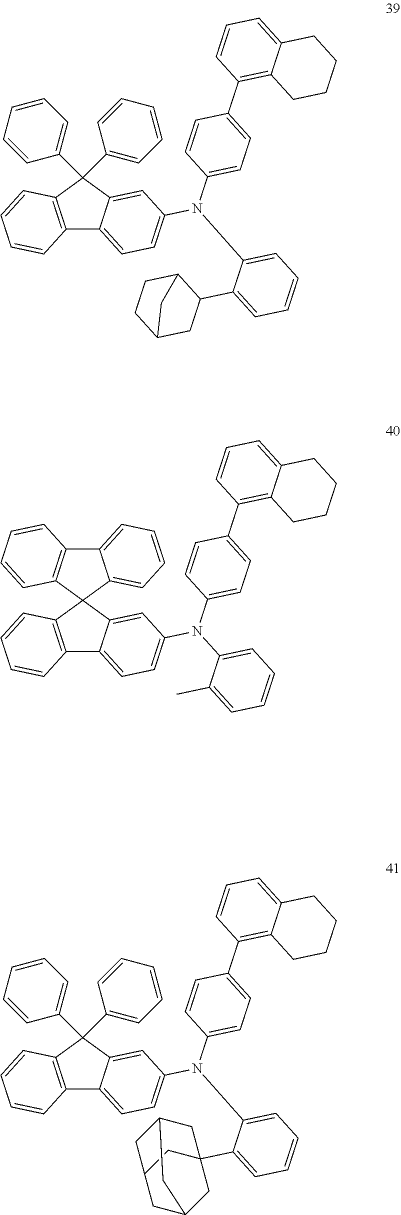 [SMILES: c1ccc(C2(c3ccccc3)c3ccccc3-c3ccc(N(c4ccc(-c5cccc6c5CCCC6)cc4)c4ccccc4C4CC5CCC4C5)cc32)cc1 ; Cc1ccccc1N(c1ccc(-c2cccc3c2CCCC3)cc1)c1ccc2c(c1)C1(c3ccccc3-c3ccccc31)c1ccccc1-2 ; c1ccc(C2(c3ccccc3)c3ccccc3-c3ccc(N(c4ccc(-c5cccc6c5CCCC6)cc4)c4ccccc4C45CC6CC(CC(C6)C4)C5)cc32)cc1]

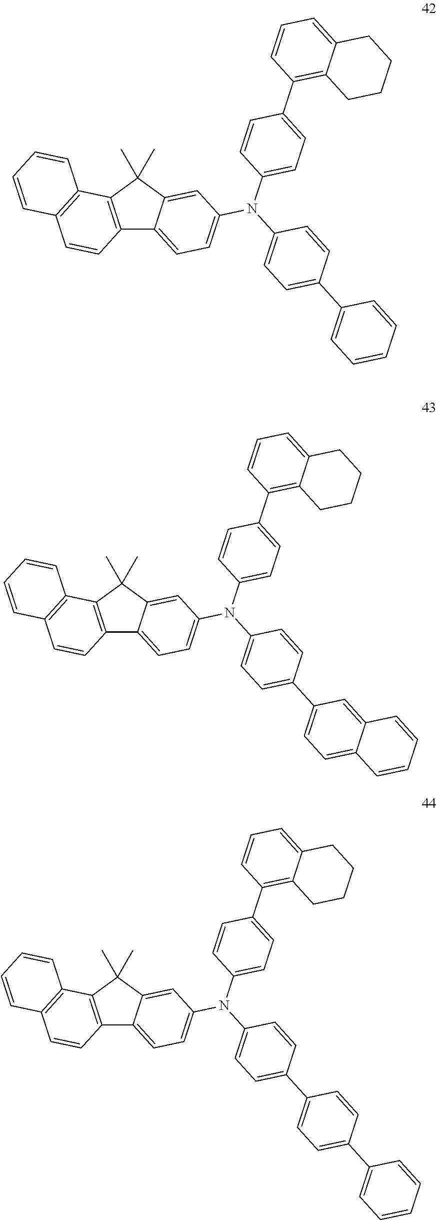 [SMILES: CC1(C)c2cc(N(c3ccc(-c4ccccc4)cc3)c3ccc(-c4cccc5c4CCCC5)cc3)ccc2-c2ccc3ccccc3c21 ; CC1(C)c2cc(N(c3ccc(-c4ccc5ccccc5c4)cc3)c3ccc(-c4cccc5c4CCCC5)cc3)ccc2-c2ccc3ccccc3c21 ; CC1(C)c2cc(N(c3ccc(-c4ccc(-c5ccccc5)cc4)cc3)c3ccc(-c4cccc5c4CCCC5)cc3)ccc2-c2ccc3ccccc3c21]

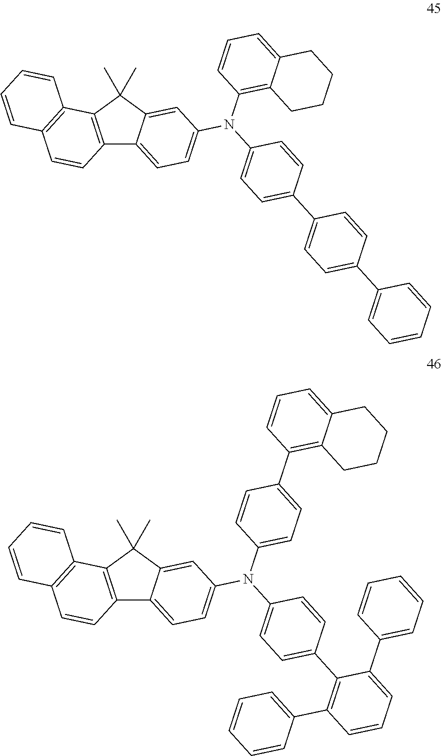 [SMILES: CC1(C)c2cc(N(c3ccc(-c4ccc(-c5ccccc5)cc4)cc3)c3cccc4c3CCCC4)ccc2-c2ccc3ccccc3c21 ; CC1(C)c2cc(N(c3ccc(-c4cccc5c4CCCC5)cc3)c3ccc(-c4c(-c5ccccc5)cccc4-c4ccccc4)cc3)ccc2-c2ccc3ccccc3c21]

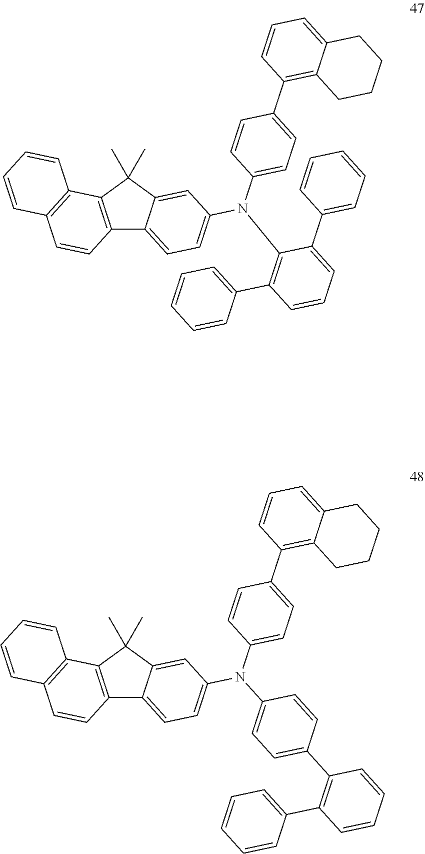 [SMILES: CC1(C)c2cc(N(c3ccc(-c4cccc5c4CCCC5)cc3)c3c(-c4ccccc4)cccc3-c3ccccc3)ccc2-c2ccc3ccccc3c21 ; CC1(C)c2cc(N(c3ccc(-c4ccccc4-c4ccccc4)cc3)c3ccc(-c4cccc5c4CCCC5)cc3)ccc2-c2ccc3ccccc3c21]

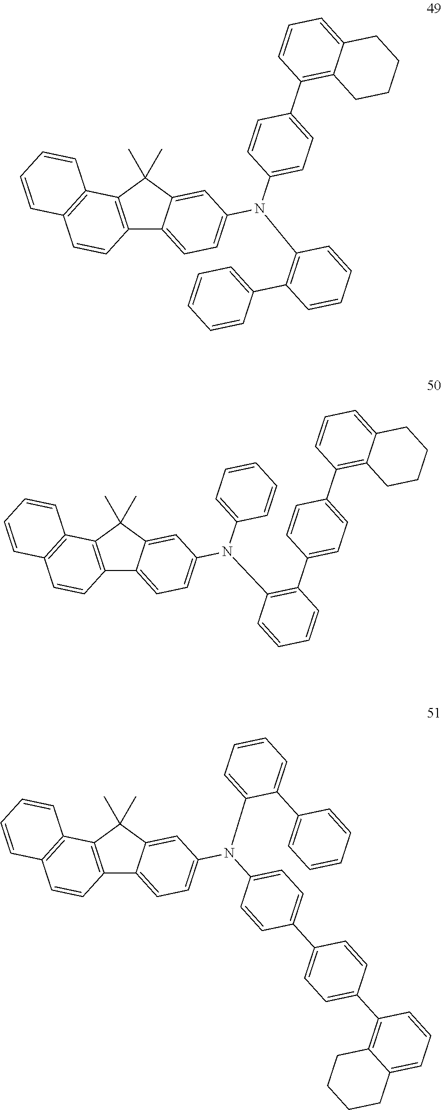 [SMILES: CC1(C)c2cc(N(c3ccc(-c4cccc5c4CCCC5)cc3)c3ccccc3-c3ccccc3)ccc2-c2ccc3ccccc3c21 ; CC1(C)c2cc(N(c3ccccc3)c3ccccc3-c3ccc(-c4cccc5c4CCCC5)cc3)ccc2-c2ccc3ccccc3c21 ; CC1(C)c2cc(N(c3ccc(-c4ccc(-c5cccc6c5CCCC6)cc4)cc3)c3ccccc3-c3ccccc3)ccc2-c2ccc3ccccc3c21]

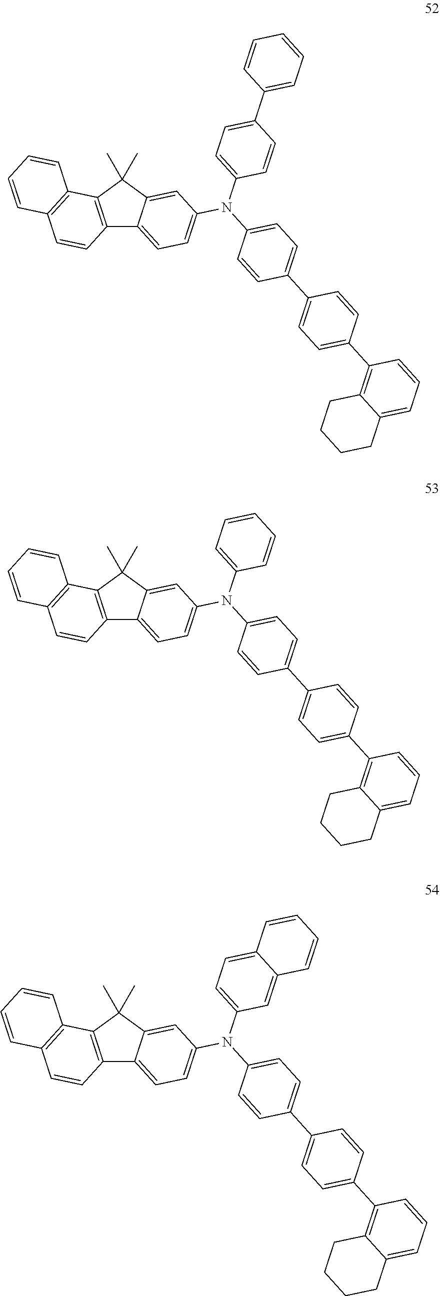 [SMILES: CC1(C)c2cc(N(c3ccc(-c4ccccc4)cc3)c3ccc(-c4ccc(-c5cccc6c5CCCC6)cc4)cc3)ccc2-c2ccc3ccccc3c21 ; CC1(C)c2cc(N(c3ccccc3)c3ccc(-c4ccc(-c5cccc6c5CCCC6)cc4)cc3)ccc2-c2ccc3ccccc3c21 ; CC1(C)c2cc(N(c3ccc(-c4ccc(-c5cccc6c5CCCC6)cc4)cc3)c3ccc4ccccc4c3)ccc2-c2ccc3ccccc3c21]

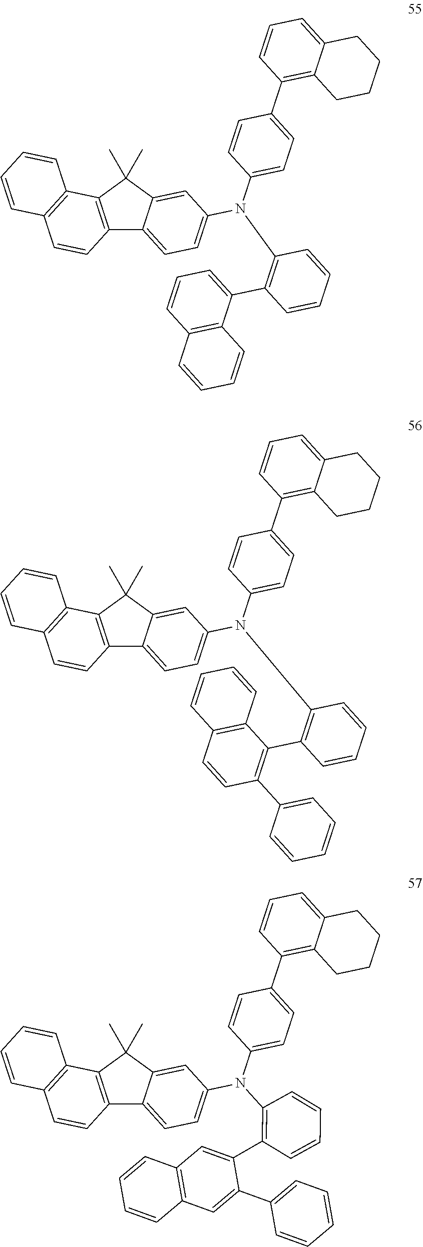

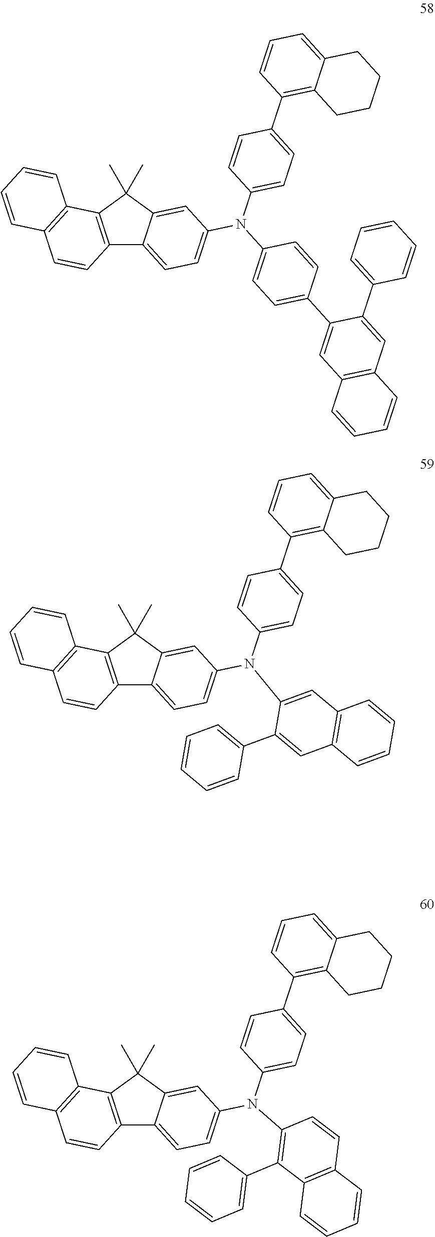 [SMILES: CC1(C)c2cc(N(c3ccc(-c4cc5ccccc5cc4-c4ccccc4)cc3)c3ccc(-c4cccc5c4CCCC5)cc3)ccc2-c2ccc3ccccc3c21 ; CC1(C)c2cc(N(c3ccc(-c4cccc5c4CCCC5)cc3)c3cc4ccccc4cc3-c3ccccc3)ccc2-c2ccc3ccccc3c21 ; CC1(C)c2cc(N(c3ccc(-c4cccc5c4CCCC5)cc3)c3ccc4ccccc4c3-c3ccccc3)ccc2-c2ccc3ccccc3c21]

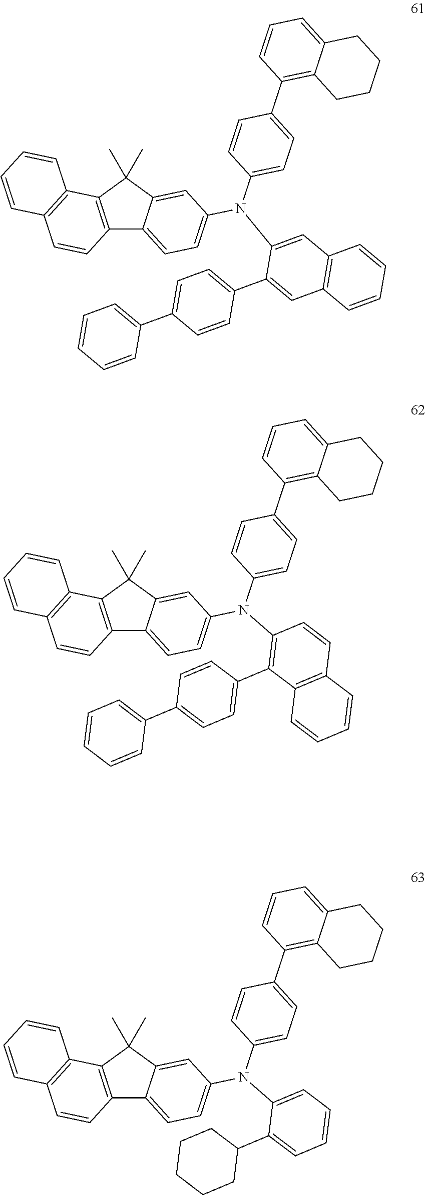 [SMILES: CC1(C)c2cc(N(c3ccc(-c4cccc5c4CCCC5)cc3)c3cc4ccccc4cc3-c3ccc(-c4ccccc4)cc3)ccc2-c2ccc3ccccc3c21 ; CC1(C)c2cc(N(c3ccc(-c4cccc5c4CCCC5)cc3)c3ccc4ccccc4c3-c3ccc(-c4ccccc4)cc3)ccc2-c2ccc3ccccc3c21 ; CC1(C)c2cc(N(c3ccc(-c4cccc5c4CCCC5)cc3)c3ccccc3C3CCCCC3)ccc2-c2ccc3ccccc3c21]

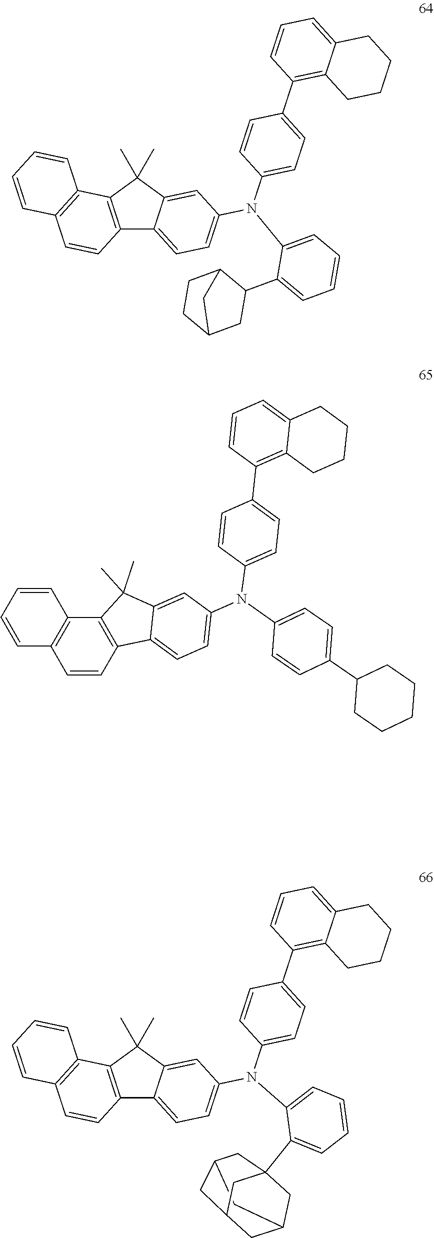 [SMILES: CC1(C)c2cc(N(c3ccc(-c4cccc5c4CCCC5)cc3)c3ccccc3C3CC4CCC3C4)ccc2-c2ccc3ccccc3c21 ; CC1(C)c2cc(N(c3ccc(-c4cccc5c4CCCC5)cc3)c3ccc(C4CCCCC4)cc3)ccc2-c2ccc3ccccc3c21 ; CC1(C)c2cc(N(c3ccc(-c4cccc5c4CCCC5)cc3)c3ccccc3C34CC5CC(CC(C5)C3)C4)ccc2-c2ccc3ccccc3c21]

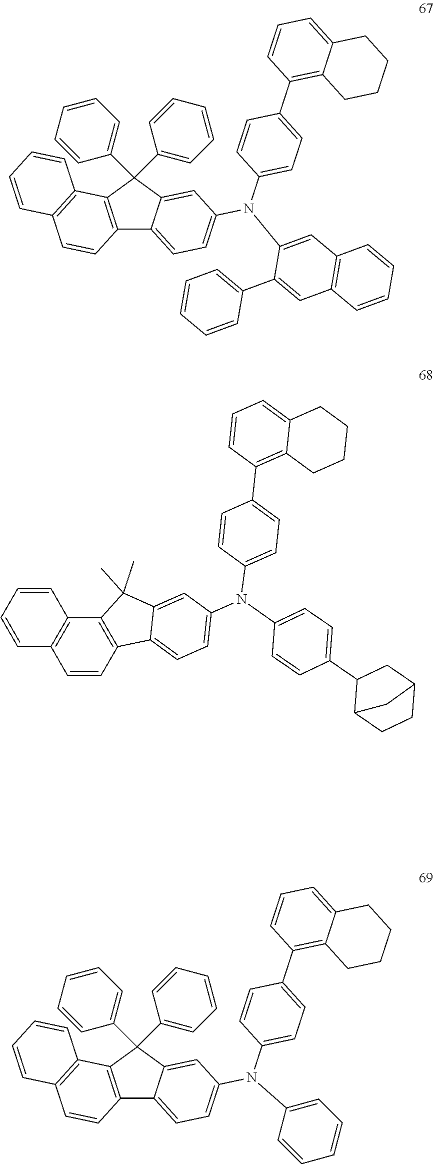 [SMILES: c1ccc(-c2cc3ccccc3cc2N(c2ccc(-c3cccc4c3CCCC4)cc2)c2ccc3c(c2)C(c2ccccc2)(c2ccccc2)c2c-3ccc3ccccc23)cc1 ; CC1(C)c2cc(N(c3ccc(-c4cccc5c4CCCC5)cc3)c3ccc(C4CC5CCC4C5)cc3)ccc2-c2ccc3ccccc3c21 ; c1ccc(N(c2ccc(-c3cccc4c3CCCC4)cc2)c2ccc3c(c2)C(c2ccccc2)(c2ccccc2)c2c-3ccc3ccccc23)cc1]

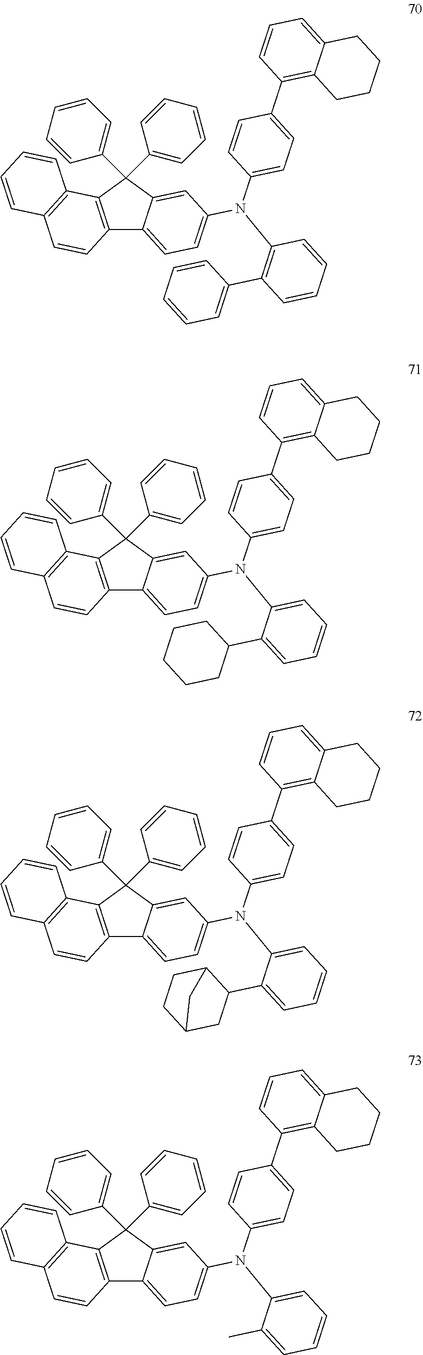 [SMILES: c1ccc(-c2ccccc2N(c2ccc(-c3cccc4c3CCCC4)cc2)c2ccc3c(c2)C(c2ccccc2)(c2ccccc2)c2c-3ccc3ccccc23)cc1 ; c1ccc(C2(c3ccccc3)c3cc(N(c4ccc(-c5cccc6c5CCCC6)cc4)c4ccccc4C4CCCCC4)ccc3-c3ccc4ccccc4c32)cc1 ; c1ccc(C2(c3ccccc3)c3cc(N(c4ccc(-c5cccc6c5CCCC6)cc4)c4ccccc4C4CC5CCC4C5)ccc3-c3ccc4ccccc4c32)cc1 ; Cc1ccccc1N(c1ccc(-c2cccc3c2CCCC3)cc1)c1ccc2c(c1)C(c1ccccc1)(c1ccccc1)c1c-2ccc2ccccc12]

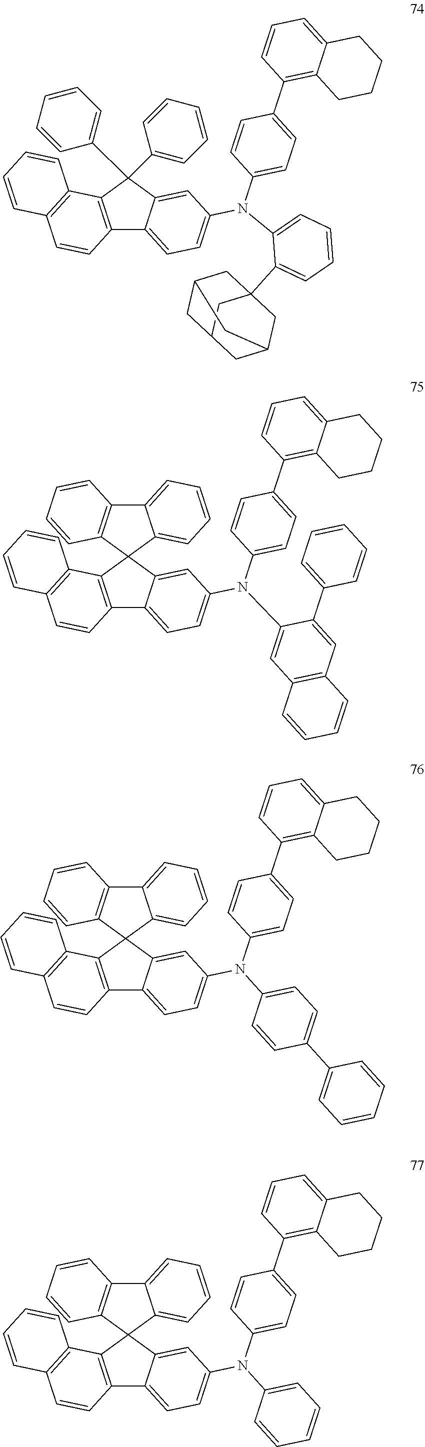

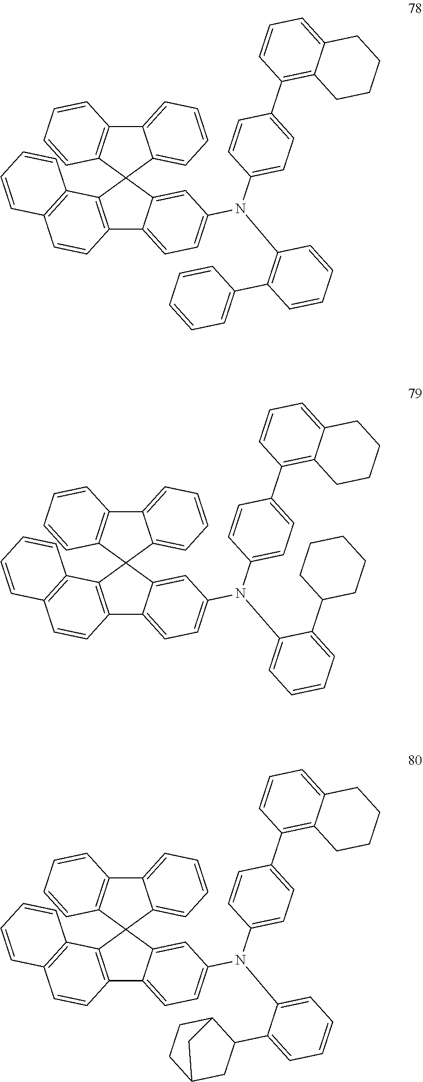 [SMILES: c1ccc(-c2ccccc2N(c2ccc(-c3cccc4c3CCCC4)cc2)c2ccc3c(c2)C2(c4ccccc4-c4ccccc42)c2c-3ccc3ccccc23)cc1 ; c1ccc2c(c1)-c1ccccc1C21c2cc(N(c3ccc(-c4cccc5c4CCCC5)cc3)c3ccccc3C3CCCCC3)ccc2-c2ccc3ccccc3c21 ; c1ccc2c(c1)-c1ccccc1C21c2cc(N(c3ccc(-c4cccc5c4CCCC5)cc3)c3ccccc3C3CC4CCC3C4)ccc2-c2ccc3ccccc3c21]

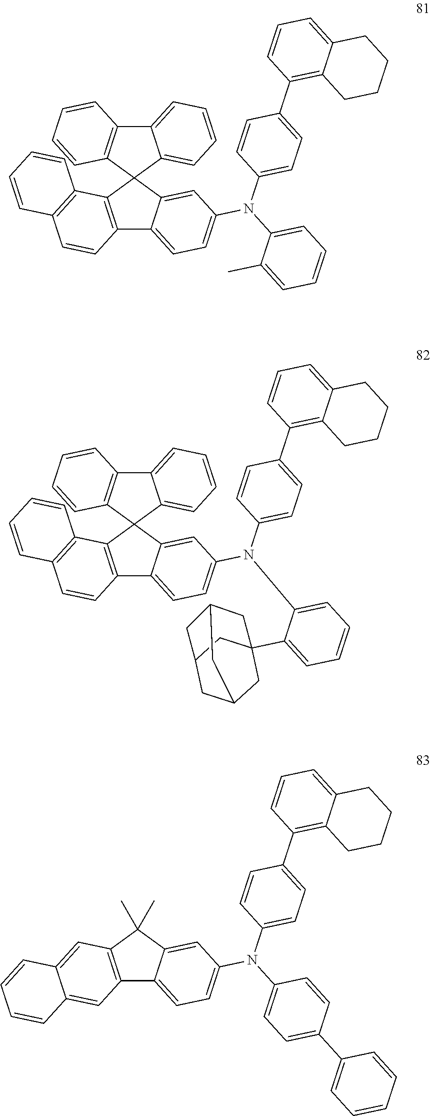 [SMILES: Cc1ccccc1N(c1ccc(-c2cccc3c2CCCC3)cc1)c1ccc2c(c1)C1(c3ccccc3-c3ccccc31)c1c-2ccc2ccccc12 ; c1ccc2c(c1)-c1ccccc1C21c2cc(N(c3ccc(-c4cccc5c4CCCC5)cc3)c3ccccc3C34CC5CC(CC(C5)C3)C4)ccc2-c2ccc3ccccc3c21 ; CC1(C)c2cc(N(c3ccc(-c4ccccc4)cc3)c3ccc(-c4cccc5c4CCCC5)cc3)ccc2-c2cc3ccccc3cc21]

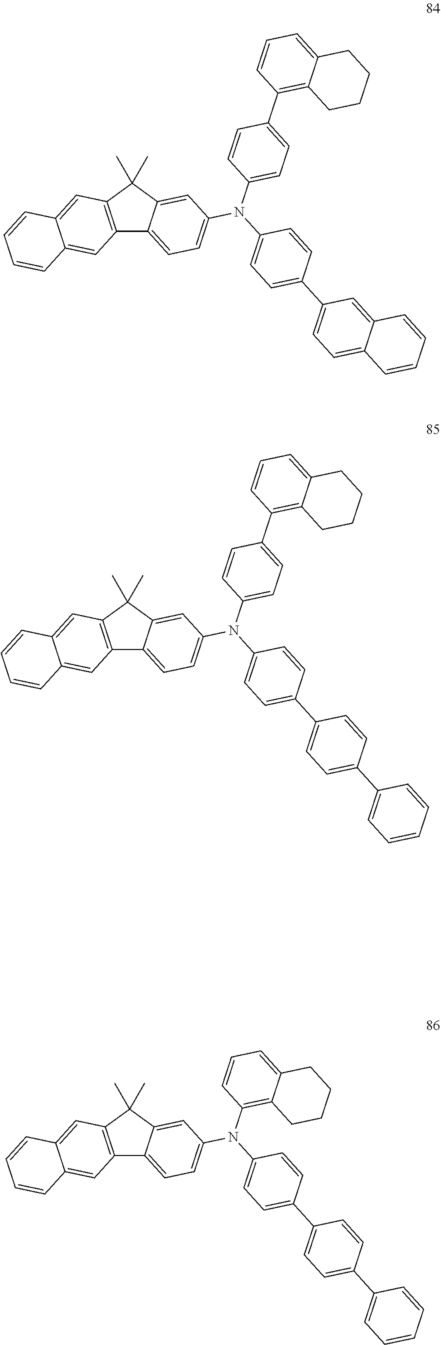 [SMILES: CC1(C)c2cc(N(c3ccc(-c4ccc5ccccc5c4)cc3)c3ccc(-c4cccc5c4CCCC5)cc3)ccc2-c2cc3ccccc3cc21 ; CC1(C)c2cc(N(c3ccc(-c4ccc(-c5ccccc5)cc4)cc3)c3ccc(-c4cccc5c4CCCC5)cc3)ccc2-c2cc3ccccc3cc21 ; CC1(C)c2cc(N(c3ccc(-c4ccc(-c5ccccc5)cc4)cc3)c3cccc4c3CCCC4)ccc2-c2cc3ccccc3cc21]

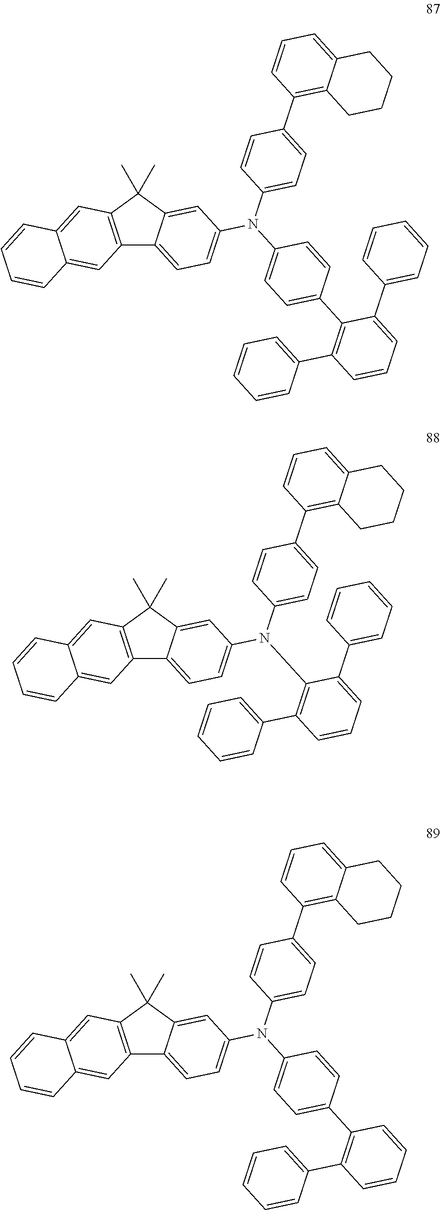 [SMILES: CC1(C)c2cc(N(c3ccc(-c4cccc5c4CCCC5)cc3)c3ccc(-c4c(-c5ccccc5)cccc4-c4ccccc4)cc3)ccc2-c2cc3ccccc3cc21 ; CC1(C)c2cc(N(c3ccc(-c4cccc5c4CCCC5)cc3)c3c(-c4ccccc4)cccc3-c3ccccc3)ccc2-c2cc3ccccc3cc21 ; CC1(C)c2cc(N(c3ccc(-c4ccccc4-c4ccccc4)cc3)c3ccc(-c4cccc5c4CCCC5)cc3)ccc2-c2cc3ccccc3cc21]

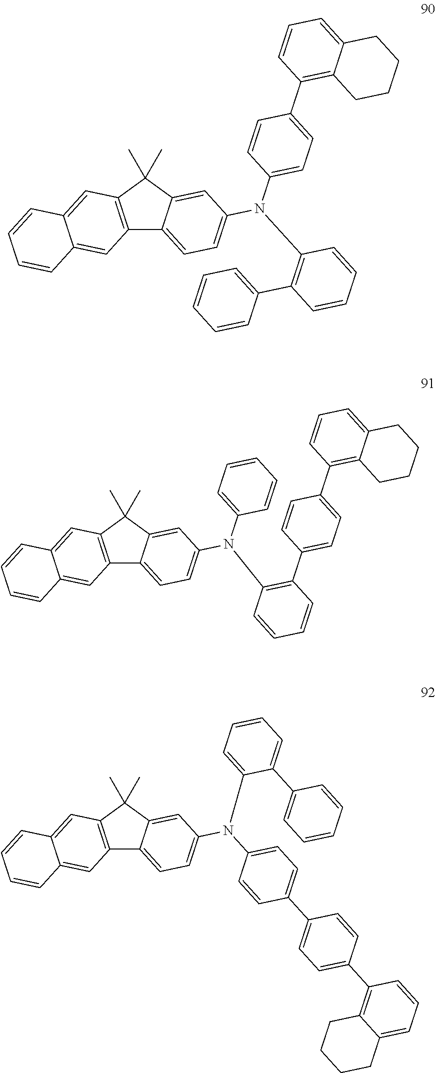 [SMILES: CC1(C)c2cc(N(c3ccc(-c4cccc5c4CCCC5)cc3)c3ccccc3-c3ccccc3)ccc2-c2cc3ccccc3cc21 ; CC1(C)c2cc(N(c3ccccc3)c3ccccc3-c3ccc(-c4cccc5c4CCCC5)cc3)ccc2-c2cc3ccccc3cc21 ; CC1(C)c2cc(N(c3ccc(-c4ccc(-c5cccc6c5CCCC6)cc4)cc3)c3ccccc3-c3ccccc3)ccc2-c2cc3ccccc3cc21]

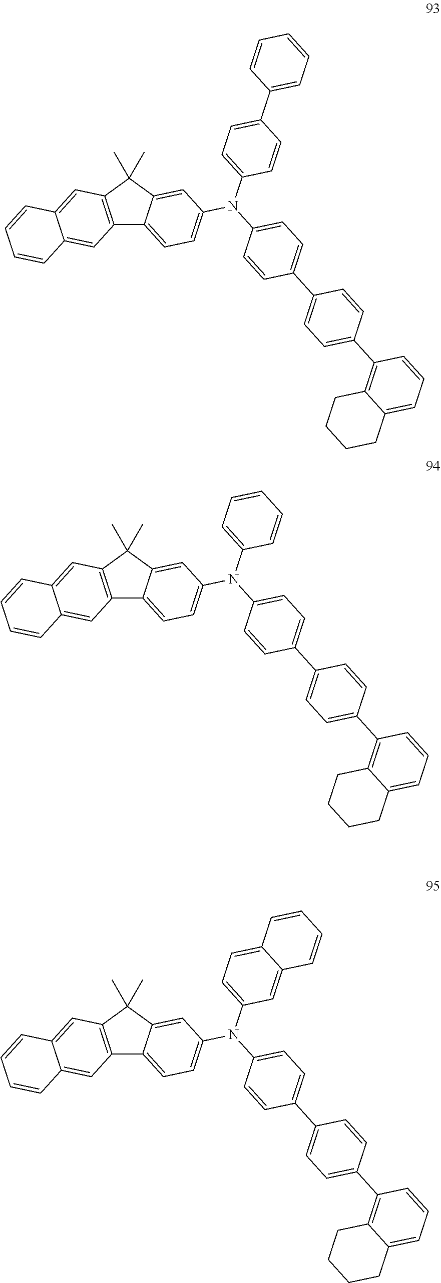 [SMILES: CC1(C)c2cc(N(c3ccc(-c4ccccc4)cc3)c3ccc(-c4ccc(-c5cccc6c5CCCC6)cc4)cc3)ccc2-c2cc3ccccc3cc21 ; CC1(C)c2cc(N(c3ccccc3)c3ccc(-c4ccc(-c5cccc6c5CCCC6)cc4)cc3)ccc2-c2cc3ccccc3cc21 ; CC1(C)c2cc(N(c3ccc(-c4ccc(-c5cccc6c5CCCC6)cc4)cc3)c3ccc4ccccc4c3)ccc2-c2cc3ccccc3cc21]

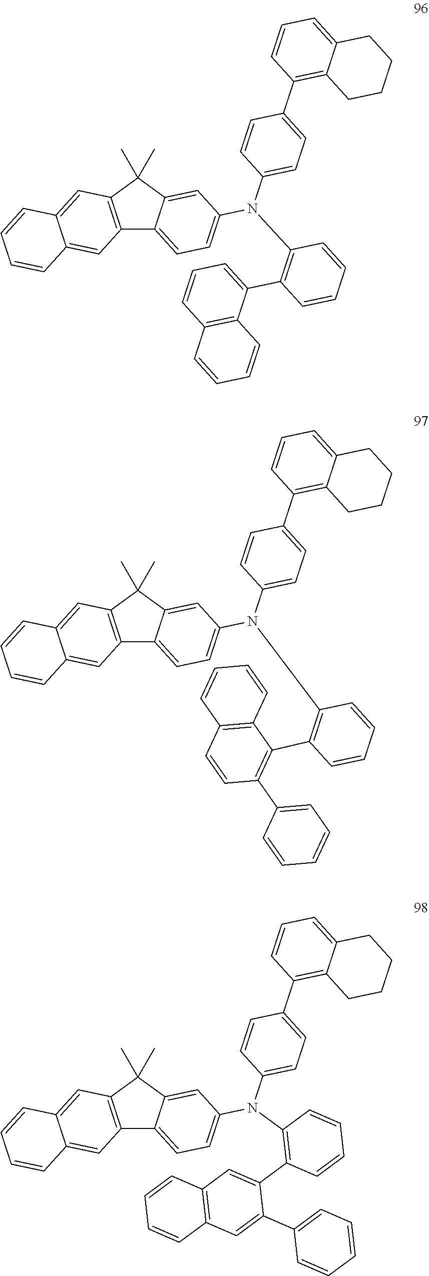 [SMILES: CC1(C)c2cc(N(c3ccc(-c4cccc5c4CCCC5)cc3)c3ccccc3-c3cccc4ccccc34)ccc2-c2cc3ccccc3cc21 ; CC1(C)c2cc(N(c3ccc(-c4cccc5c4CCCC5)cc3)c3ccccc3-c3c(-c4ccccc4)ccc4ccccc34)ccc2-c2cc3ccccc3cc21 ; CC1(C)c2cc(N(c3ccc(-c4cccc5c4CCCC5)cc3)c3ccccc3-c3cc4ccccc4cc3-c3ccccc3)ccc2-c2cc3ccccc3cc21]

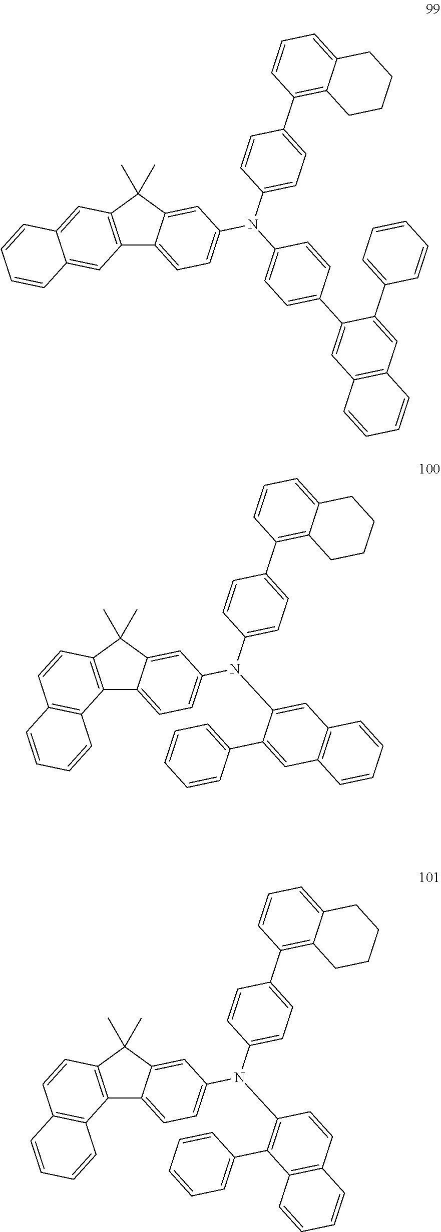 [SMILES: CC1(C)c2cc(N(c3ccc(-c4cc5ccccc5cc4-c4ccccc4)cc3)c3ccc(-c4cccc5c4CCCC5)cc3)ccc2-c2cc3ccccc3cc21 ; CC1(C)c2cc(N(c3ccc(-c4cccc5c4CCCC5)cc3)c3cc4ccccc4cc3-c3ccccc3)ccc2-c2c1ccc1ccccc21 ; CC1(C)c2cc(N(c3ccc(-c4cccc5c4CCCC5)cc3)c3ccc4ccccc4c3-c3ccccc3)ccc2-c2c1ccc1ccccc21]

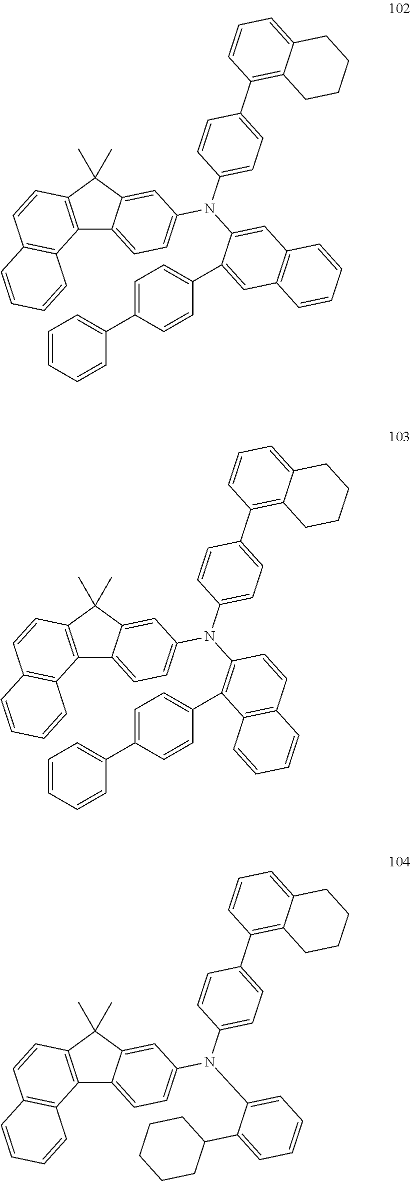 [SMILES: CC1(C)c2cc(N(c3ccc(-c4cccc5c4CCCC5)cc3)c3cc4ccccc4cc3-c3ccc(-c4ccccc4)cc3)ccc2-c2c1ccc1ccccc21 ; CC1(C)c2cc(N(c3ccc(-c4cccc5c4CCCC5)cc3)c3ccc4ccccc4c3-c3ccc(-c4ccccc4)cc3)ccc2-c2c1ccc1ccccc21 ; CC1(C)c2cc(N(c3ccc(-c4cccc5c4CCCC5)cc3)c3ccccc3C3CCCCC3)ccc2-c2c1ccc1ccccc21]

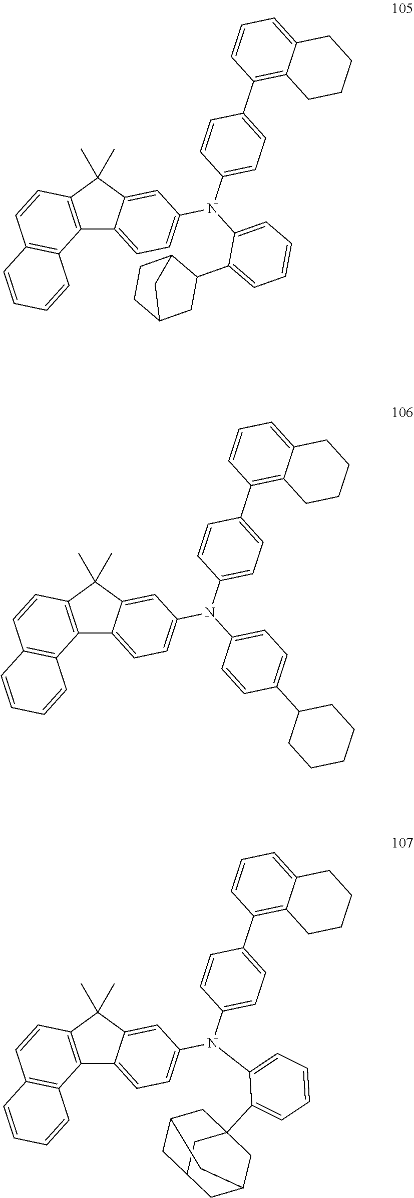 [SMILES: CC1(C)c2cc(N(c3ccc(-c4cccc5c4CCCC5)cc3)c3ccccc3C3CC4CCC3C4)ccc2-c2c1ccc1ccccc21 ; CC1(C)c2cc(N(c3ccc(-c4cccc5c4CCCC5)cc3)c3ccc(C4CCCCC4)cc3)ccc2-c2c1ccc1ccccc21 ; CC1(C)c2cc(N(c3ccc(-c4cccc5c4CCCC5)cc3)c3ccccc3C34CC5CC(CC(C5)C3)C4)ccc2-c2c1ccc1ccccc21]

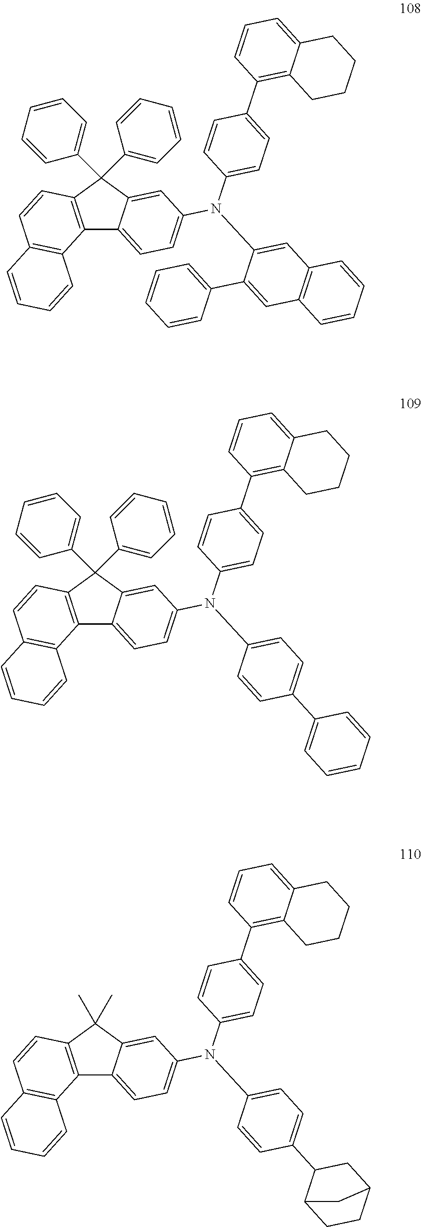 [SMILES: c1ccc(-c2cc3ccccc3cc2N(c2ccc(-c3cccc4c3CCCC4)cc2)c2ccc3c(c2)C(c2ccccc2)(c2ccccc2)c2ccc4ccccc4c2-3)cc1 ; c1ccc(-c2ccc(N(c3ccc(-c4cccc5c4CCCC5)cc3)c3ccc4c(c3)C(c3ccccc3)(c3ccccc3)c3ccc5ccccc5c3-4)cc2)cc1 ; CC1(C)c2cc(N(c3ccc(-c4cccc5c4CCCC5)cc3)c3ccc(C4CC5CCC4C5)cc3)ccc2-c2c1ccc1ccccc21]

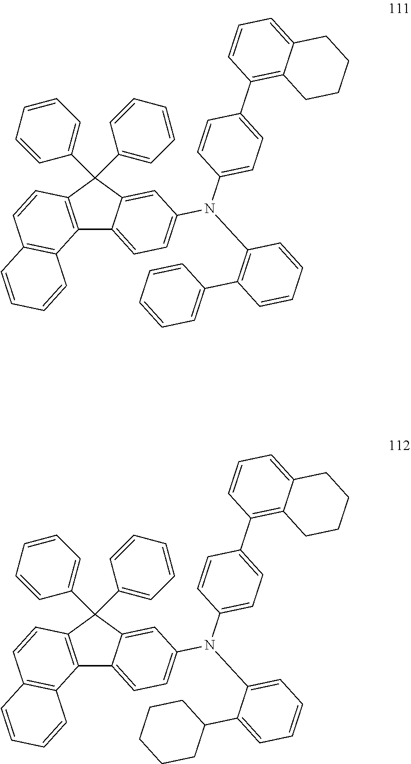 [SMILES: c1ccc(-c2ccccc2N(c2ccc(-c3cccc4c3CCCC4)cc2)c2ccc3c(c2)C(c2ccccc2)(c2ccccc2)c2ccc4ccccc4c2-3)cc1 ; c1ccc(C2(c3ccccc3)c3cc(N(c4ccc(-c5cccc6c5CCCC6)cc4)c4ccccc4C4CCCCC4)ccc3-c3c2ccc2ccccc32)cc1]

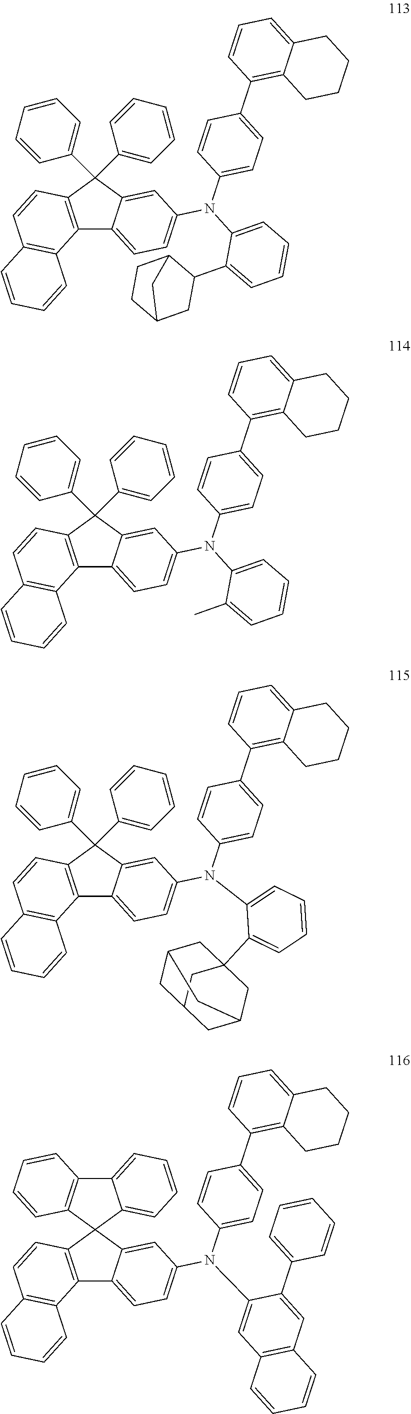 [SMILES: c1ccc(C2(c3ccccc3)c3cc(N(c4ccc(-c5cccc6c5CCCC6)cc4)c4ccccc4C4CC5CCC4C5)ccc3-c3c2ccc2ccccc32)cc1 ; Cc1ccccc1N(c1ccc(-c2cccc3c2CCCC3)cc1)c1ccc2c(c1)C(c1ccccc1)(c1ccccc1)c1ccc3ccccc3c1-2 ; c1ccc(C2(c3ccccc3)c3cc(N(c4ccc(-c5cccc6c5CCCC6)cc4)c4ccccc4C45CC6CC(CC(C6)C4)C5)ccc3-c3c2ccc2ccccc32)cc1 ; c1ccc(-c2cc3ccccc3cc2N(c2ccc(-c3cccc4c3CCCC4)cc2)c2ccc3c(c2)C2(c4ccccc4-c4ccccc42)c2ccc4ccccc4c2-3)cc1]

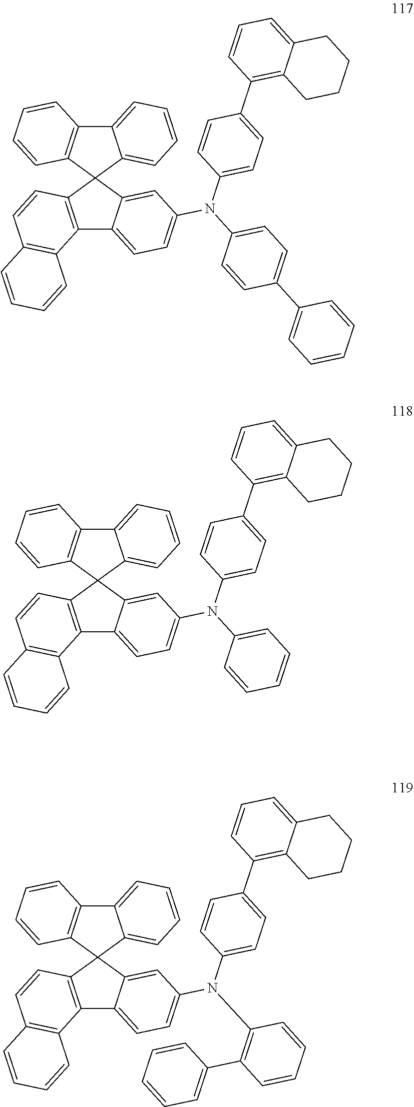 [SMILES: c1ccc(-c2ccc(N(c3ccc(-c4cccc5c4CCCC5)cc3)c3ccc4c(c3)C3(c5ccccc5-c5ccccc53)c3ccc5ccccc5c3-4)cc2)cc1 ; c1ccc(N(c2ccc(-c3cccc4c3CCCC4)cc2)c2ccc3c(c2)C2(c4ccccc4-c4ccccc42)c2ccc4ccccc4c2-3)cc1 ; c1ccc(-c2ccccc2N(c2ccc(-c3cccc4c3CCCC4)cc2)c2ccc3c(c2)C2(c4ccccc4-c4ccccc42)c2ccc4ccccc4c2-3)cc1]

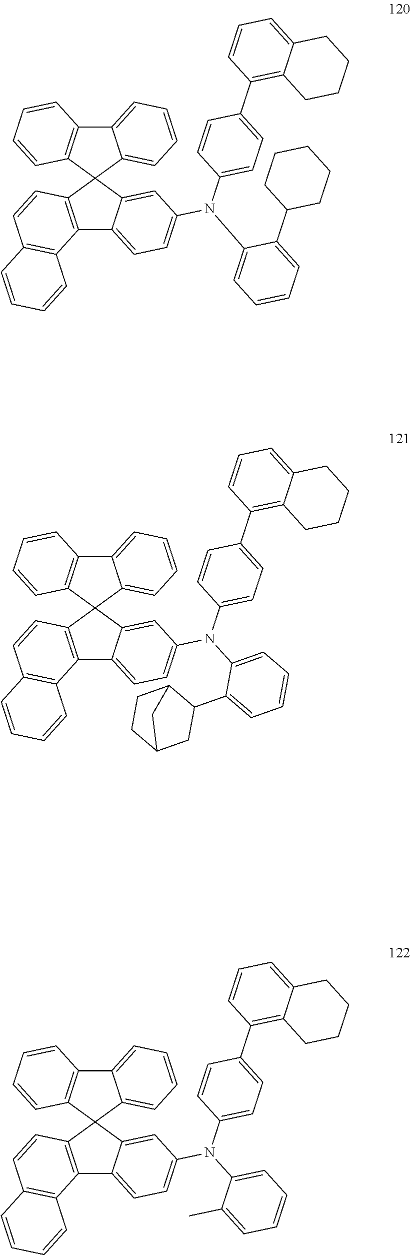 [SMILES: c1ccc2c(c1)-c1ccccc1C21c2cc(N(c3ccc(-c4cccc5c4CCCC5)cc3)c3ccccc3C3CCCCC3)ccc2-c2c1ccc1ccccc21 ; c1ccc2c(c1)-c1ccccc1C21c2cc(N(c3ccc(-c4cccc5c4CCCC5)cc3)c3ccccc3C3CC4CCC3C4)ccc2-c2c1ccc1ccccc21 ; Cc1ccccc1N(c1ccc(-c2cccc3c2CCCC3)cc1)c1ccc2c(c1)C1(c3ccccc3-c3ccccc31)c1ccc3ccccc3c1-2]

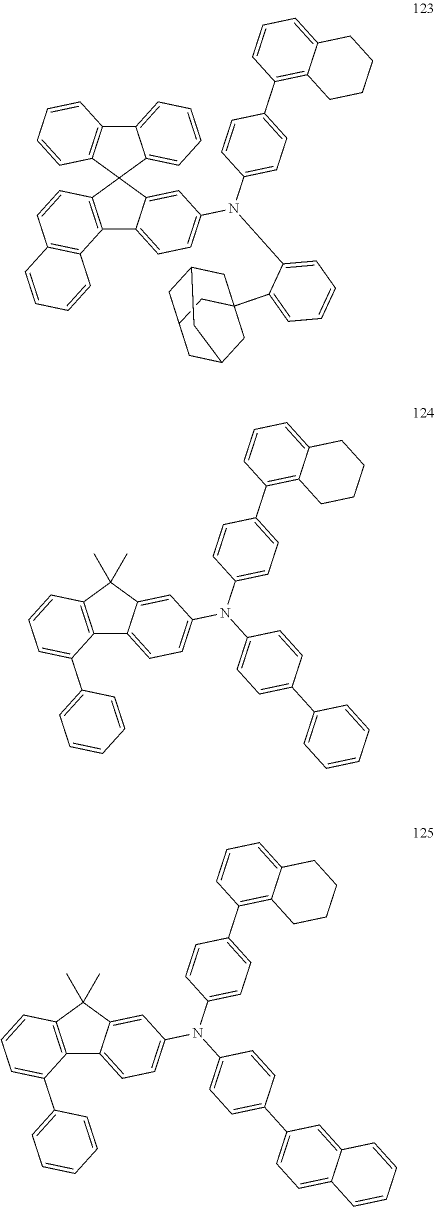 [SMILES: c1ccc2c(c1)-c1ccccc1C21c2cc(N(c3ccc(-c4cccc5c4CCCC5)cc3)c3ccccc3C34CC5CC(CC(C5)C3)C4)ccc2-c2c1ccc1ccccc21 ; CC1(C)c2cc(N(c3ccc(-c4ccccc4)cc3)c3ccc(-c4cccc5c4CCCC5)cc3)ccc2-c2c(-c3ccccc3)cccc21 ; CC1(C)c2cc(N(c3ccc(-c4ccc5ccccc5c4)cc3)c3ccc(-c4cccc5c4CCCC5)cc3)ccc2-c2c(-c3ccccc3)cccc21]

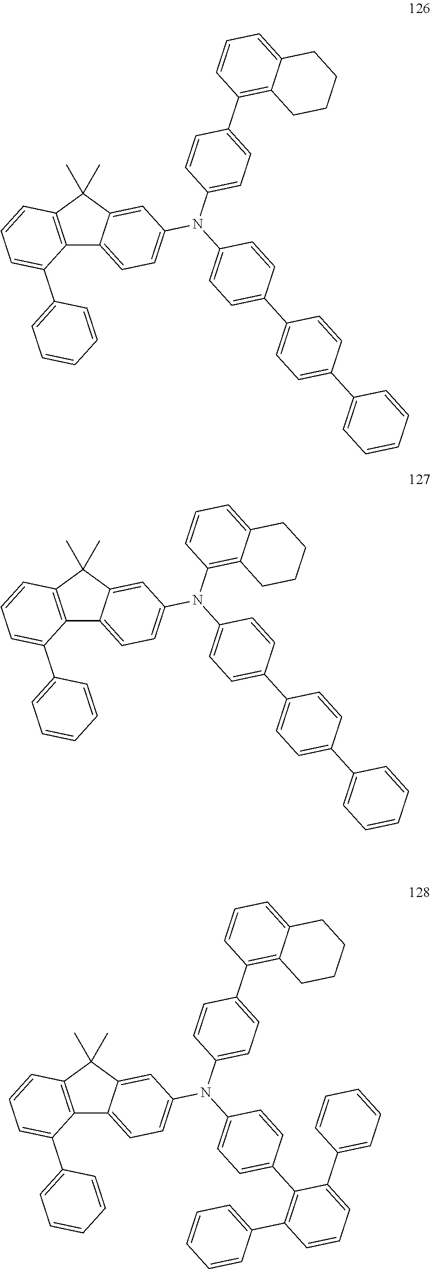 [SMILES: CC1(C)c2cc(N(c3ccc(-c4ccc(-c5ccccc5)cc4)cc3)c3ccc(-c4cccc5c4CCCC5)cc3)ccc2-c2c(-c3ccccc3)cccc21 ; CC1(C)c2cc(N(c3ccc(-c4ccc(-c5ccccc5)cc4)cc3)c3cccc4c3CCCC4)ccc2-c2c(-c3ccccc3)cccc21 ; CC1(C)c2cc(N(c3ccc(-c4cccc5c4CCCC5)cc3)c3ccc(-c4c(-c5ccccc5)cccc4-c4ccccc4)cc3)ccc2-c2c(-c3ccccc3)cccc21]

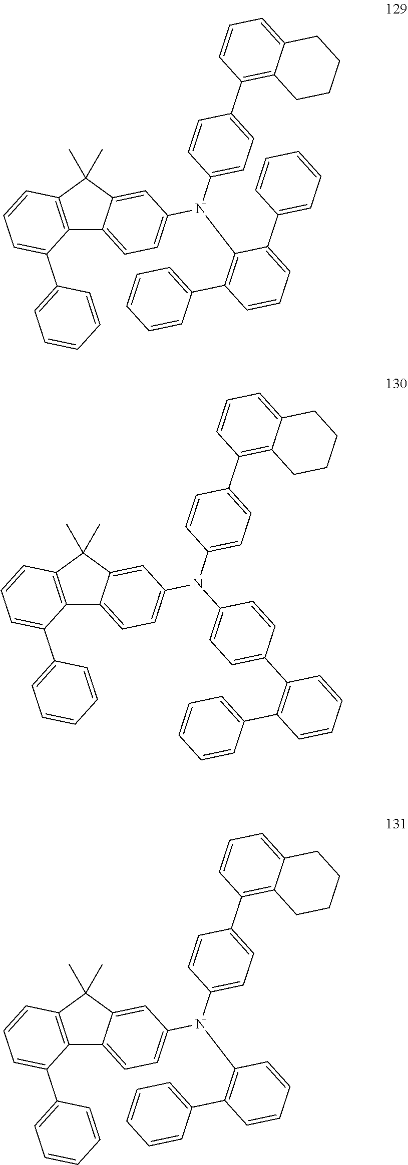 [SMILES: CC1(C)c2cc(N(c3ccc(-c4cccc5c4CCCC5)cc3)c3c(-c4ccccc4)cccc3-c3ccccc3)ccc2-c2c(-c3ccccc3)cccc21 ; CC1(C)c2cc(N(c3ccc(-c4ccccc4-c4ccccc4)cc3)c3ccc(-c4cccc5c4CCCC5)cc3)ccc2-c2c(-c3ccccc3)cccc21 ; CC1(C)c2cc(N(c3ccc(-c4cccc5c4CCCC5)cc3)c3ccccc3-c3ccccc3)ccc2-c2c(-c3ccccc3)cccc21]

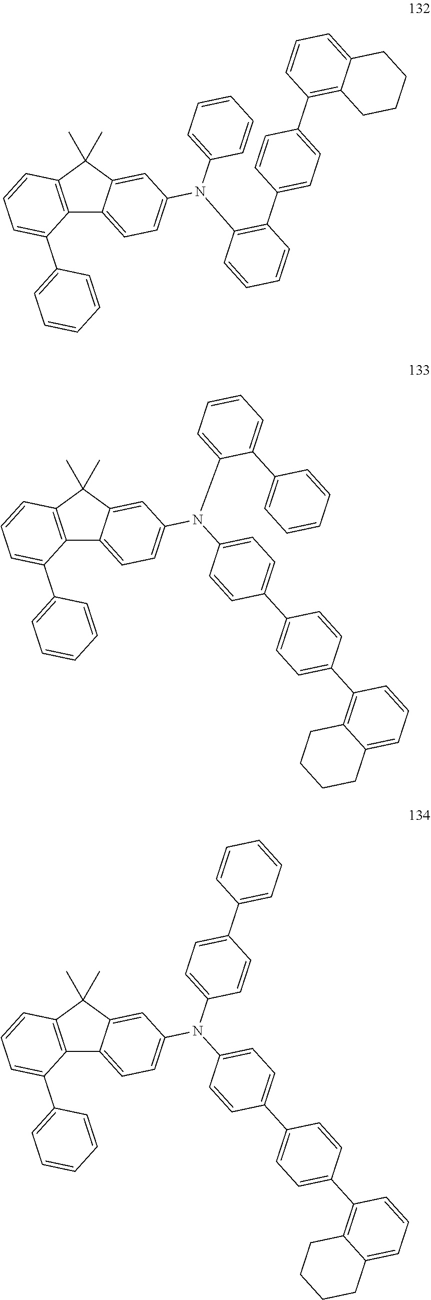 [SMILES: CC1(C)c2cc(N(c3ccccc3)c3ccccc3-c3ccc(-c4cccc5c4CCCC5)cc3)ccc2-c2c(-c3ccccc3)cccc21 ; CC1(C)c2cc(N(c3ccc(-c4ccc(-c5cccc6c5CCCC6)cc4)cc3)c3ccccc3-c3ccccc3)ccc2-c2c(-c3ccccc3)cccc21 ; CC1(C)c2cc(N(c3ccc(-c4ccccc4)cc3)c3ccc(-c4ccc(-c5cccc6c5CCCC6)cc4)cc3)ccc2-c2c(-c3ccccc3)cccc21]

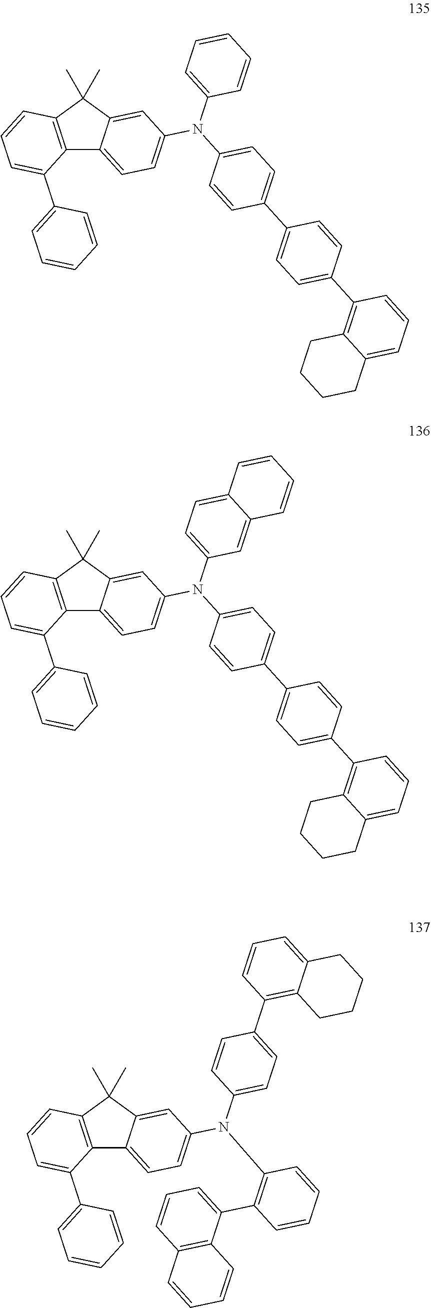 [SMILES: CC1(C)c2cc(N(c3ccccc3)c3ccc(-c4ccc(-c5cccc6c5CCCC6)cc4)cc3)ccc2-c2c(-c3ccccc3)cccc21 ; CC1(C)c2cc(N(c3ccc(-c4ccc(-c5cccc6c5CCCC6)cc4)cc3)c3ccc4ccccc4c3)ccc2-c2c(-c3ccccc3)cccc21 ; CC1(C)c2cc(N(c3ccc(-c4cccc5c4CCCC5)cc3)c3ccccc3-c3cccc4ccccc34)ccc2-c2c(-c3ccccc3)cccc21]

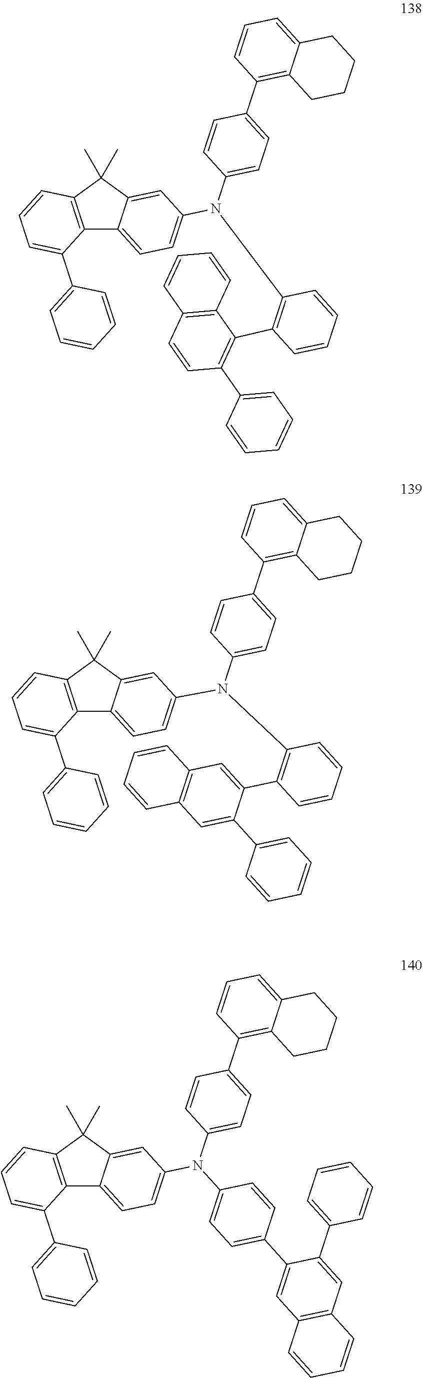 [SMILES: CC1(C)c2cc(N(c3ccc(-c4cccc5c4CCCC5)cc3)c3ccccc3-c3c(-c4ccccc4)ccc4ccccc34)ccc2-c2c(-c3ccccc3)cccc21 ; CC1(C)c2cc(N(c3ccc(-c4cccc5c4CCCC5)cc3)c3ccccc3-c3cc4ccccc4cc3-c3ccccc3)ccc2-c2c(-c3ccccc3)cccc21 ; CC1(C)c2cc(N(c3ccc(-c4cc5ccccc5cc4-c4ccccc4)cc3)c3ccc(-c4cccc5c4CCCC5)cc3)ccc2-c2c(-c3ccccc3)cccc21]

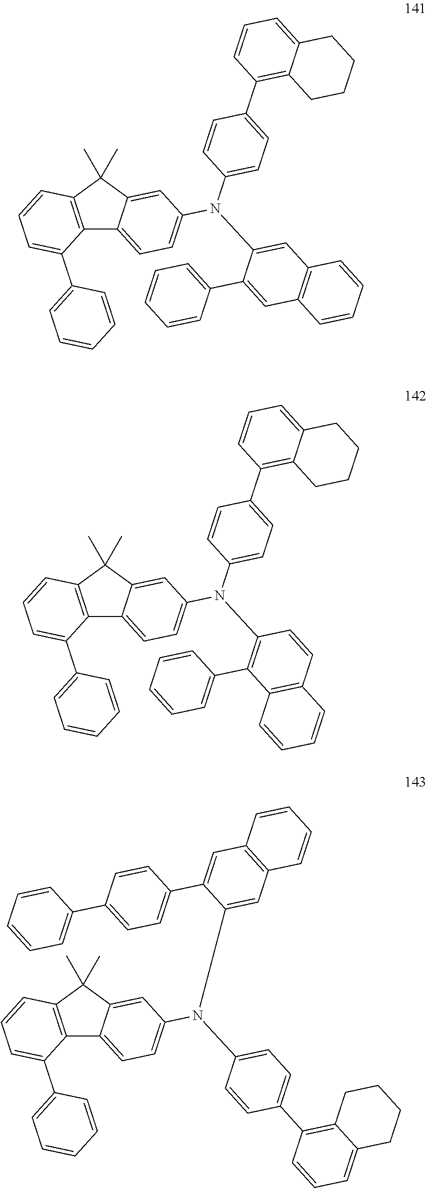 [SMILES: CC1(C)c2cc(N(c3ccc(-c4cccc5c4CCCC5)cc3)c3cc4ccccc4cc3-c3ccccc3)ccc2-c2c(-c3ccccc3)cccc21 ; CC1(C)c2cc(N(c3ccc(-c4cccc5c4CCCC5)cc3)c3ccc4ccccc4c3-c3ccccc3)ccc2-c2c(-c3ccccc3)cccc21 ; CC1(C)c2cc(N(c3ccc(-c4cccc5c4CCCC5)cc3)c3cc4ccccc4cc3-c3ccc(-c4ccccc4)cc3)ccc2-c2c(-c3ccccc3)cccc21]

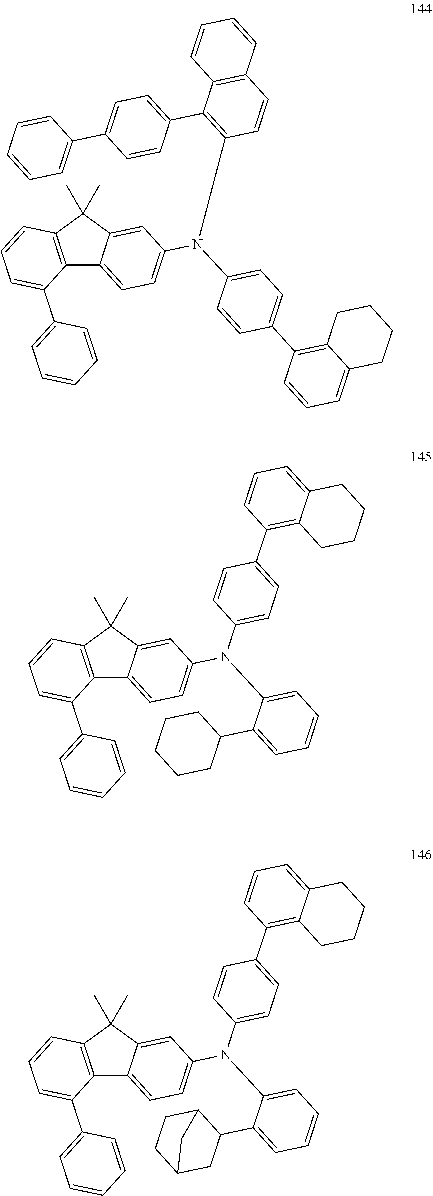 [SMILES: CC1(C)c2cc(N(c3ccc(-c4cccc5c4CCCC5)cc3)c3ccc4ccccc4c3-c3ccc(-c4ccccc4)cc3)ccc2-c2c(-c3ccccc3)cccc21 ; CC1(C)c2cc(N(c3ccc(-c4cccc5c4CCCC5)cc3)c3ccccc3C3CCCCC3)ccc2-c2c(-c3ccccc3)cccc21 ; CC1(C)c2cc(N(c3ccc(-c4cccc5c4CCCC5)cc3)c3ccccc3C3CC4CCC3C4)ccc2-c2c(-c3ccccc3)cccc21]

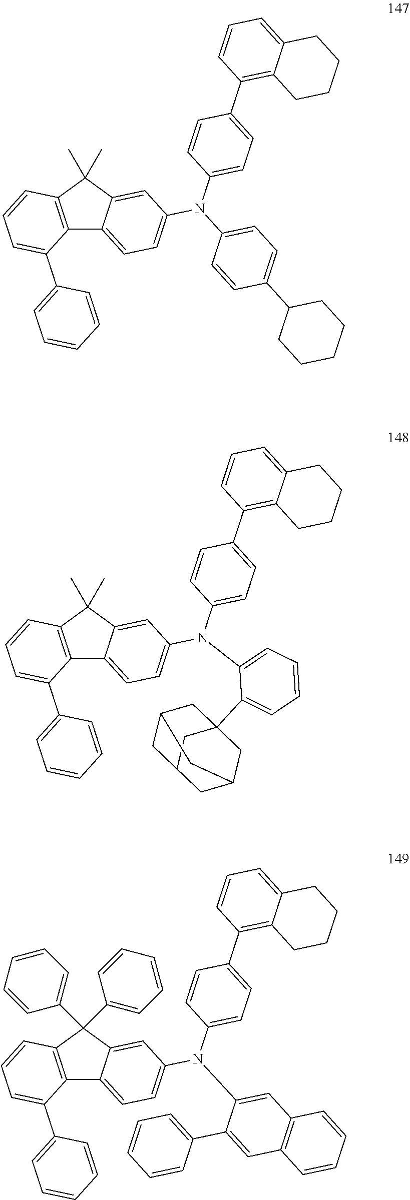 [SMILES: CC1(C)c2cc(N(c3ccc(-c4cccc5c4CCCC5)cc3)c3ccc(C4CCCCC4)cc3)ccc2-c2c(-c3ccccc3)cccc21 ; CC1(C)c2cc(N(c3ccc(-c4cccc5c4CCCC5)cc3)c3ccccc3C34CC5CC(CC(C5)C3)C4)ccc2-c2c(-c3ccccc3)cccc21 ; c1ccc(-c2cc3ccccc3cc2N(c2ccc(-c3cccc4c3CCCC4)cc2)c2ccc3c(c2)C(c2ccccc2)(c2ccccc2)c2cccc(-c4ccccc4)c2-3)cc1]

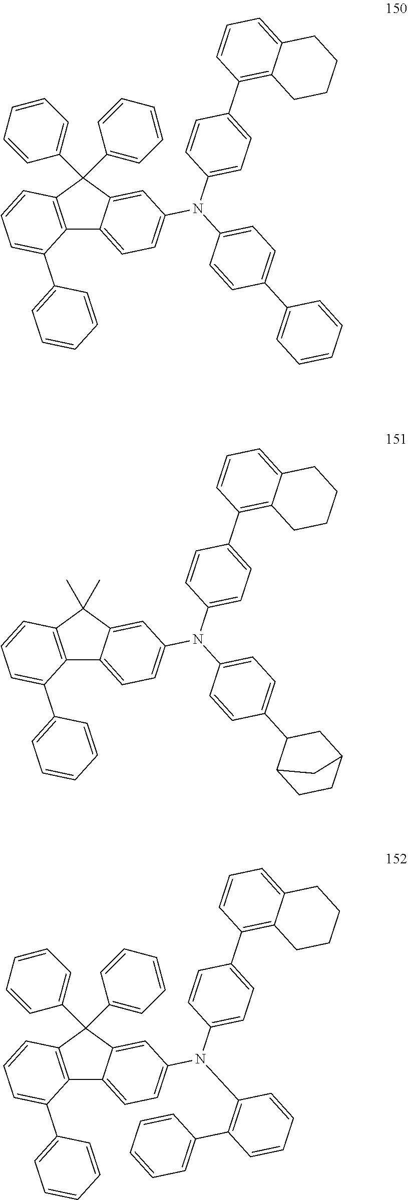 [SMILES: c1ccc(-c2ccc(N(c3ccc(-c4cccc5c4CCCC5)cc3)c3ccc4c(c3)C(c3ccccc3)(c3ccccc3)c3cccc(-c5ccccc5)c3-4)cc2)cc1 ; CC1(C)c2cc(N(c3ccc(-c4cccc5c4CCCC5)cc3)c3ccc(C4CC5CCC4C5)cc3)ccc2-c2c(-c3ccccc3)cccc21 ; c1ccc(-c2ccccc2N(c2ccc(-c3cccc4c3CCCC4)cc2)c2ccc3c(c2)C(c2ccccc2)(c2ccccc2)c2cccc(-c4ccccc4)c2-3)cc1]

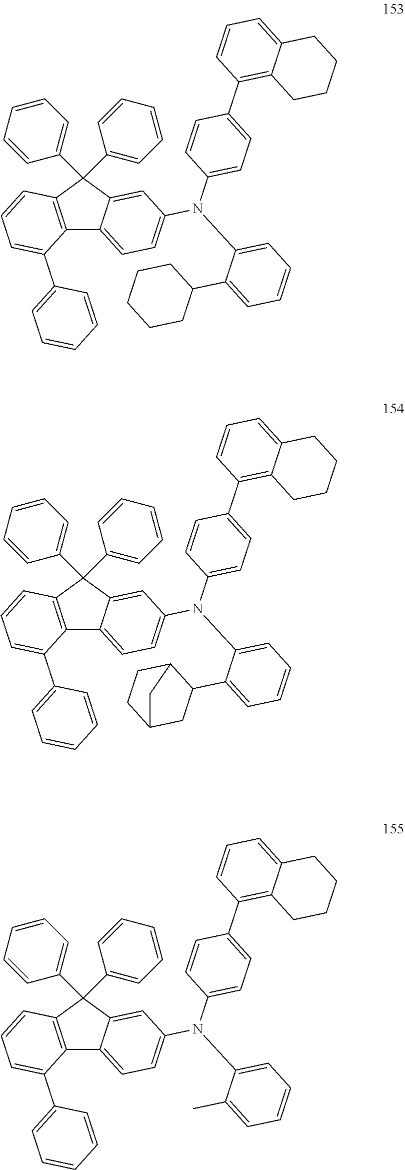 [SMILES: c1ccc(-c2cccc3c2-c2ccc(N(c4ccc(-c5cccc6c5CCCC6)cc4)c4ccccc4C4CCCCC4)cc2C3(c2ccccc2)c2ccccc2)cc1 ; c1ccc(-c2cccc3c2-c2ccc(N(c4ccc(-c5cccc6c5CCCC6)cc4)c4ccccc4C4CC5CCC4C5)cc2C3(c2ccccc2)c2ccccc2)cc1 ; Cc1ccccc1N(c1ccc(-c2cccc3c2CCCC3)cc1)c1ccc2c(c1)C(c1ccccc1)(c1ccccc1)c1cccc(-c3ccccc3)c1-2]

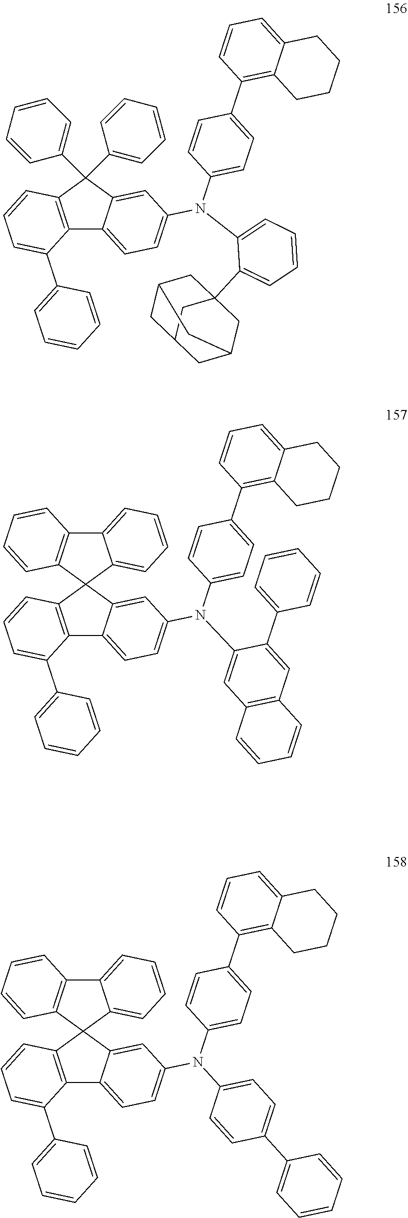 [SMILES: c1ccc(-c2cccc3c2-c2ccc(N(c4ccc(-c5cccc6c5CCCC6)cc4)c4ccccc4C45CC6CC(CC(C6)C4)C5)cc2C3(c2ccccc2)c2ccccc2)cc1 ; c1ccc(-c2cc3ccccc3cc2N(c2ccc(-c3cccc4c3CCCC4)cc2)c2ccc3c(c2)C2(c4ccccc4-c4ccccc42)c2cccc(-c4ccccc4)c2-3)cc1 ; c1ccc(-c2ccc(N(c3ccc(-c4cccc5c4CCCC5)cc3)c3ccc4c(c3)C3(c5ccccc5-c5ccccc53)c3cccc(-c5ccccc5)c3-4)cc2)cc1]

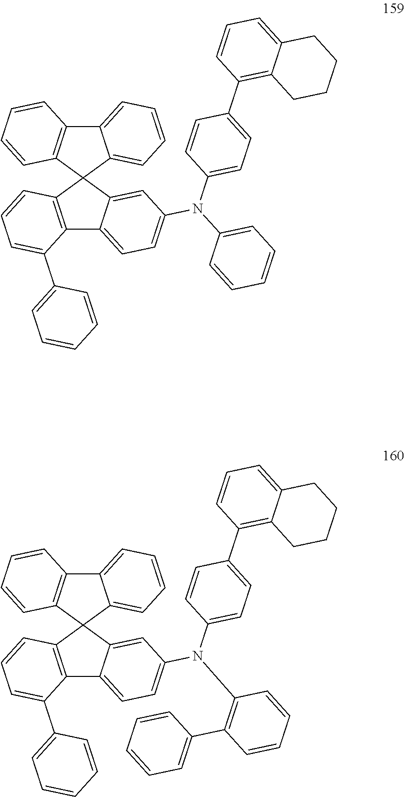 [SMILES: c1ccc(-c2cccc3c2-c2ccc(N(c4ccccc4)c4ccc(-c5cccc6c5CCCC6)cc4)cc2C32c3ccccc3-c3ccccc32)cc1 ; c1ccc(-c2ccccc2N(c2ccc(-c3cccc4c3CCCC4)cc2)c2ccc3c(c2)C2(c4ccccc4-c4ccccc42)c2cccc(-c4ccccc4)c2-3)cc1]

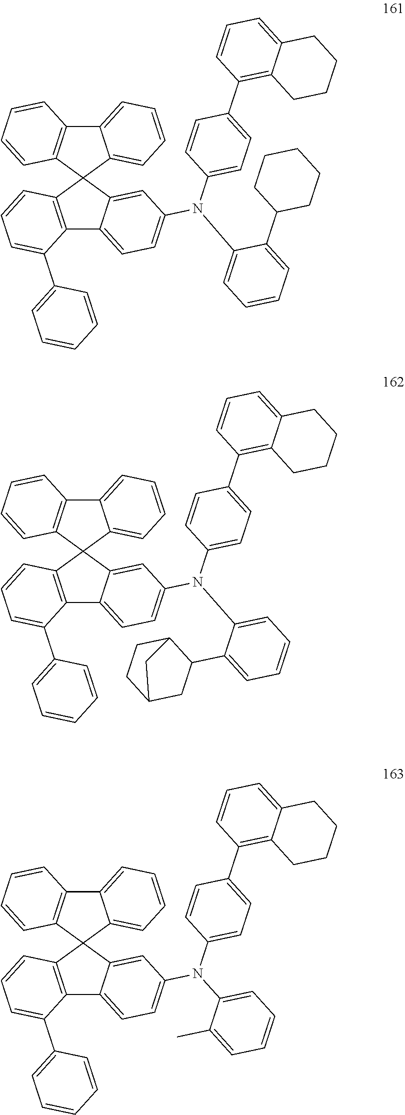 [SMILES: c1ccc(-c2cccc3c2-c2ccc(N(c4ccc(-c5cccc6c5CCCC6)cc4)c4ccccc4C4CCCCC4)cc2C32c3ccccc3-c3ccccc32)cc1 ; c1ccc(-c2cccc3c2-c2ccc(N(c4ccc(-c5cccc6c5CCCC6)cc4)c4ccccc4C4CC5CCC4C5)cc2C32c3ccccc3-c3ccccc32)cc1 ; Cc1ccccc1N(c1ccc(-c2cccc3c2CCCC3)cc1)c1ccc2c(c1)C1(c3ccccc3-c3ccccc31)c1cccc(-c3ccccc3)c1-2]

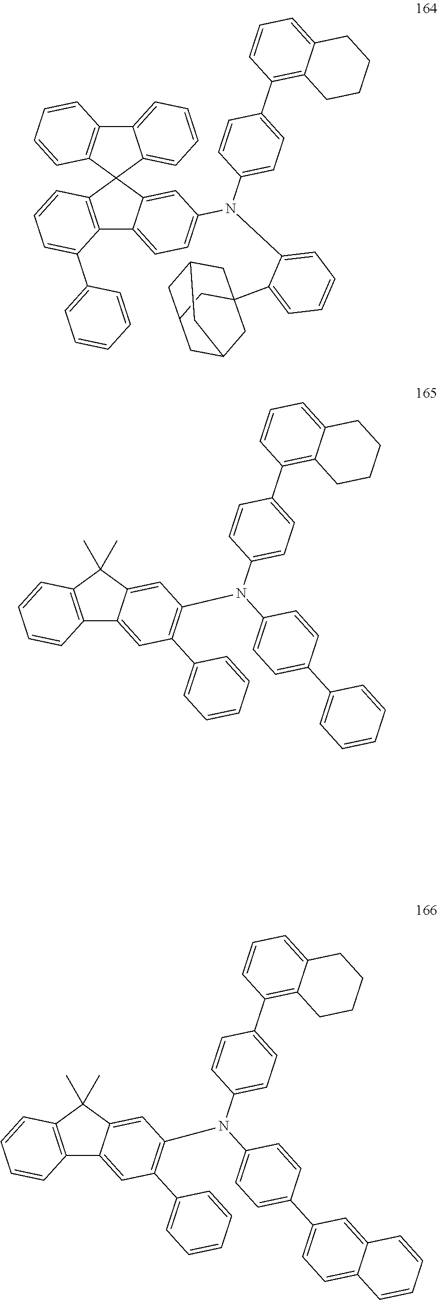 [SMILES: c1ccc(-c2cccc3c2-c2ccc(N(c4ccc(-c5cccc6c5CCCC6)cc4)c4ccccc4C45CC6CC(CC(C6)C4)C5)cc2C32c3ccccc3-c3ccccc32)cc1 ; CC1(C)c2ccccc2-c2cc(-c3ccccc3)c(N(c3ccc(-c4ccccc4)cc3)c3ccc(-c4cccc5c4CCCC5)cc3)cc21 ; CC1(C)c2ccccc2-c2cc(-c3ccccc3)c(N(c3ccc(-c4ccc5ccccc5c4)cc3)c3ccc(-c4cccc5c4CCCC5)cc3)cc21]

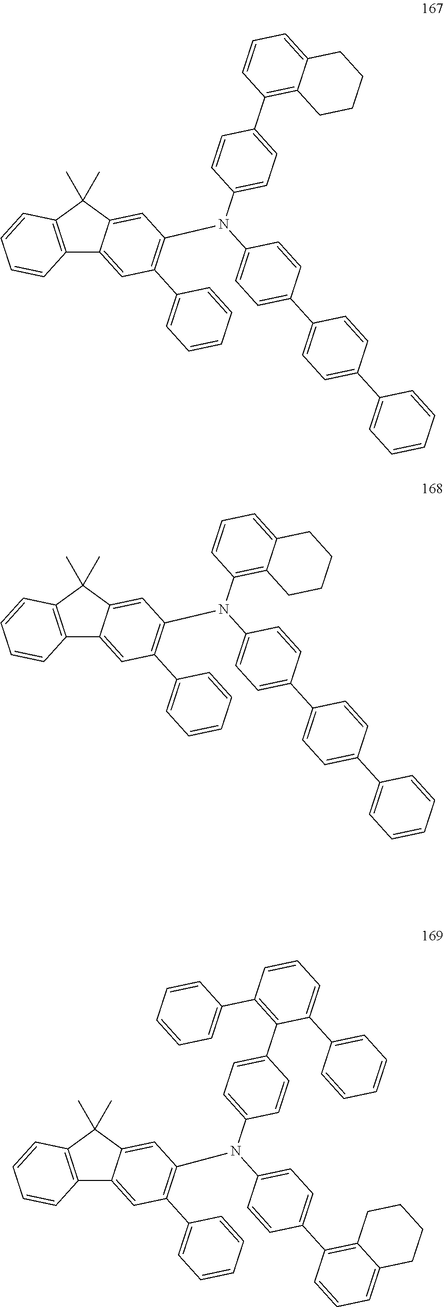 [SMILES: CC1(C)c2ccccc2-c2cc(-c3ccccc3)c(N(c3ccc(-c4ccc(-c5ccccc5)cc4)cc3)c3ccc(-c4cccc5c4CCCC5)cc3)cc21 ; CC1(C)c2ccccc2-c2cc(-c3ccccc3)c(N(c3ccc(-c4ccc(-c5ccccc5)cc4)cc3)c3cccc4c3CCCC4)cc21 ; CC1(C)c2ccccc2-c2cc(-c3ccccc3)c(N(c3ccc(-c4cccc5c4CCCC5)cc3)c3ccc(-c4c(-c5ccccc5)cccc4-c4ccccc4)cc3)cc21]

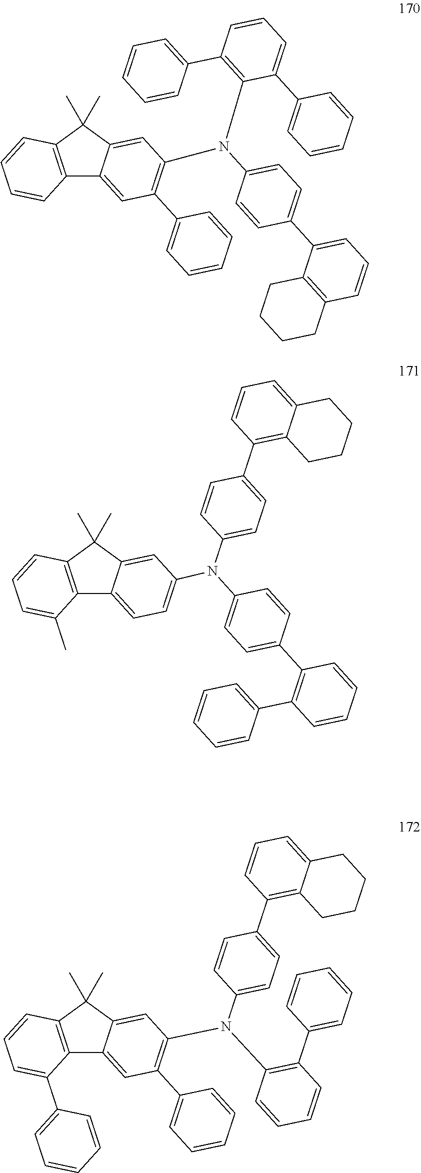

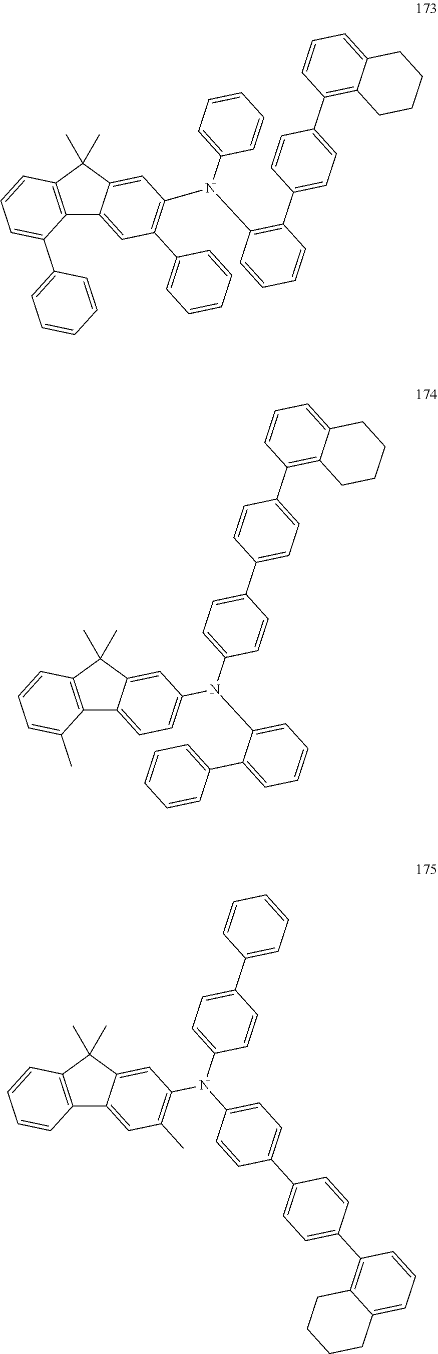 [SMILES: CC1(C)c2cc(N(c3ccccc3)c3ccccc3-c3ccc(-c4cccc5c4CCCC5)cc3)c(-c3ccccc3)cc2-c2c(-c3ccccc3)cccc21 ; Cc1cccc2c1-c1ccc(N(c3ccc(-c4ccc(-c5cccc6c5CCCC6)cc4)cc3)c3ccccc3-c3ccccc3)cc1C2(C)C ; Cc1cc2c(cc1N(c1ccc(-c3ccccc3)cc1)c1ccc(-c3ccc(-c4cccc5c4CCCC5)cc3)cc1)C(C)(C)c1ccccc1-2]

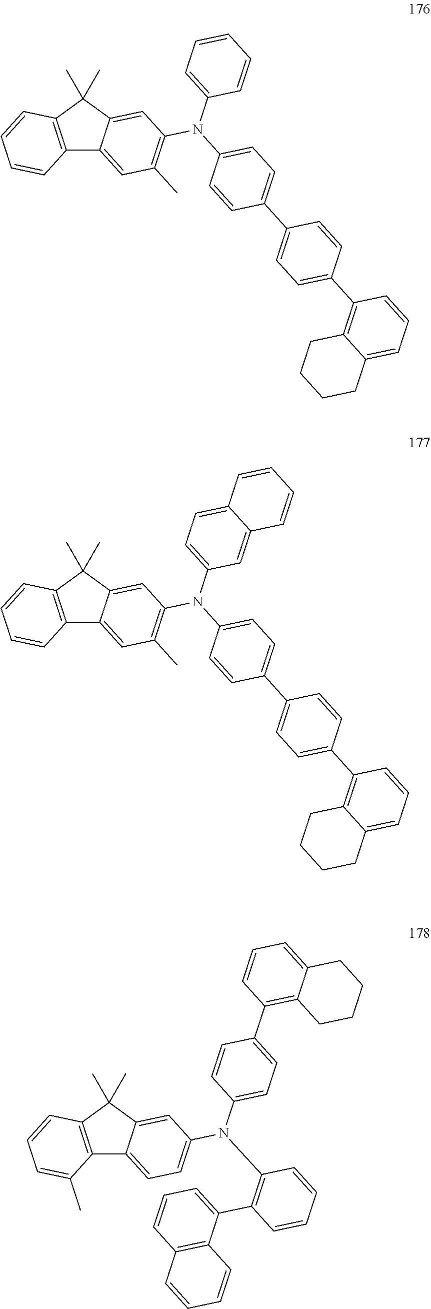 [SMILES: Cc1cc2c(cc1N(c1ccccc1)c1ccc(-c3ccc(-c4cccc5c4CCCC5)cc3)cc1)C(C)(C)c1ccccc1-2 ; Cc1cc2c(cc1N(c1ccc(-c3ccc(-c4cccc5c4CCCC5)cc3)cc1)c1ccc3ccccc3c1)C(C)(C)c1ccccc1-2 ; Cc1cccc2c1-c1ccc(N(c3ccc(-c4cccc5c4CCCC5)cc3)c3ccccc3-c3cccc4ccccc34)cc1C2(C)C]

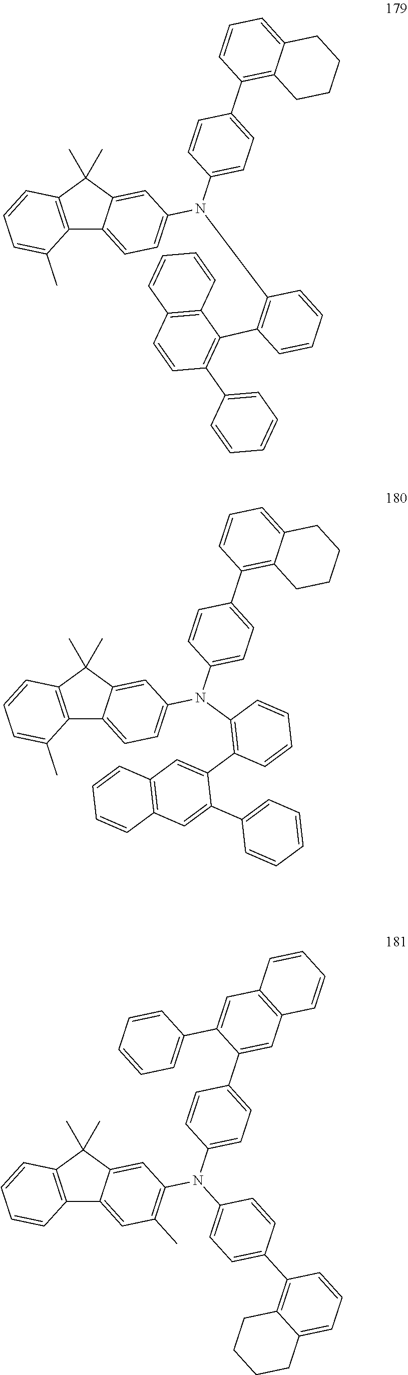 [SMILES: Cc1cccc2c1-c1ccc(N(c3ccc(-c4cccc5c4CCCC5)cc3)c3ccccc3-c3c(-c4ccccc4)ccc4ccccc34)cc1C2(C)C ; Cc1cccc2c1-c1ccc(N(c3ccc(-c4cccc5c4CCCC5)cc3)c3ccccc3-c3cc4ccccc4cc3-c3ccccc3)cc1C2(C)C ; Cc1cc2c(cc1N(c1ccc(-c3cc4ccccc4cc3-c3ccccc3)cc1)c1ccc(-c3cccc4c3CCCC4)cc1)C(C)(C)c1ccccc1-2]

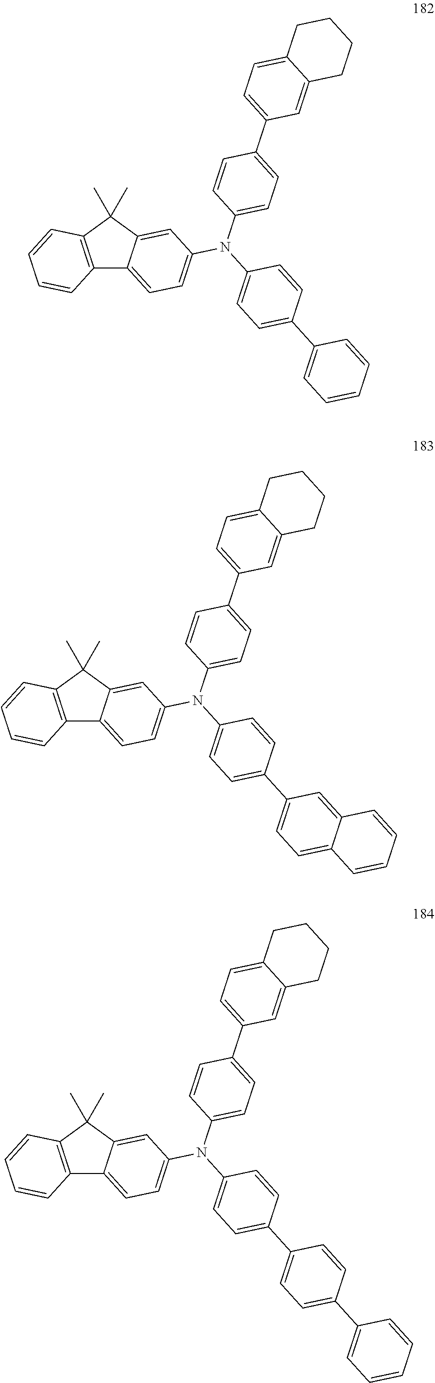 [SMILES: CC1(C)c2ccccc2-c2ccc(N(c3ccc(-c4ccccc4)cc3)c3ccc(-c4ccc5c(c4)CCCC5)cc3)cc21 ; CC1(C)c2ccccc2-c2ccc(N(c3ccc(-c4ccc5c(c4)CCCC5)cc3)c3ccc(-c4ccc5ccccc5c4)cc3)cc21 ; CC1(C)c2ccccc2-c2ccc(N(c3ccc(-c4ccc(-c5ccccc5)cc4)cc3)c3ccc(-c4ccc5c(c4)CCCC5)cc3)cc21]

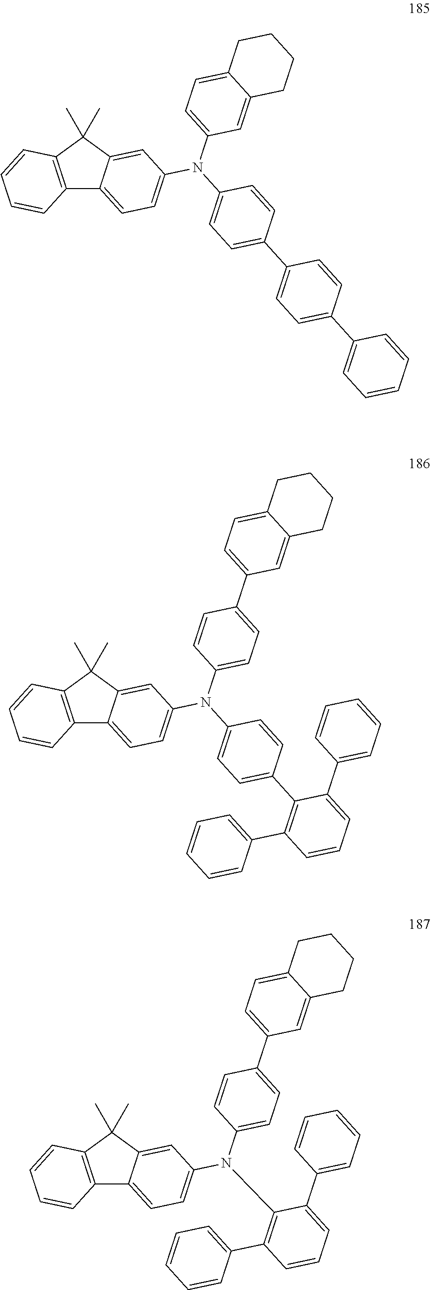 [SMILES: CC1(C)c2ccccc2-c2ccc(N(c3ccc(-c4ccc(-c5ccccc5)cc4)cc3)c3ccc4c(c3)CCCC4)cc21 ; CC1(C)c2ccccc2-c2ccc(N(c3ccc(-c4ccc5c(c4)CCCC5)cc3)c3ccc(-c4c(-c5ccccc5)cccc4-c4ccccc4)cc3)cc21 ; CC1(C)c2ccccc2-c2ccc(N(c3ccc(-c4ccc5c(c4)CCCC5)cc3)c3c(-c4ccccc4)cccc3-c3ccccc3)cc21]

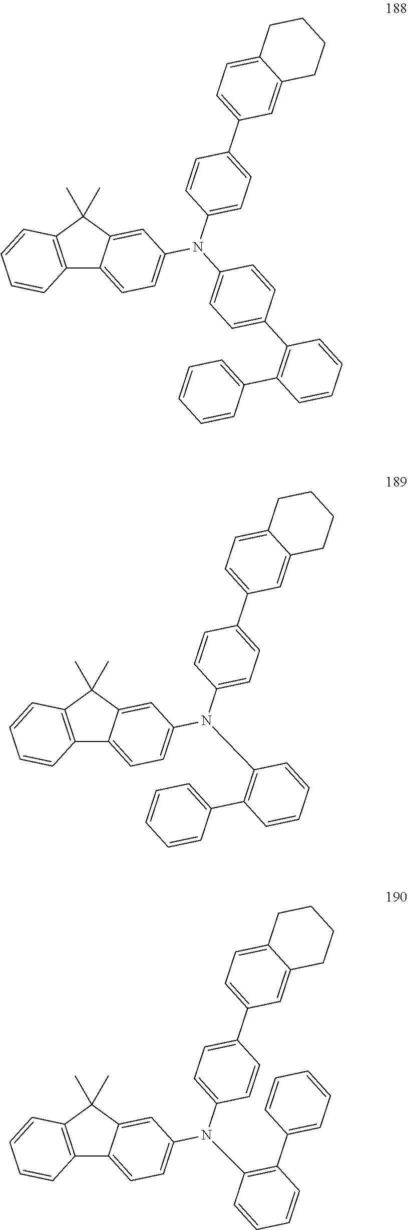 [SMILES: CC1(C)c2ccccc2-c2ccc(N(c3ccc(-c4ccc5c(c4)CCCC5)cc3)c3ccc(-c4ccccc4-c4ccccc4)cc3)cc21 ; CC1(C)c2ccccc2-c2ccc(N(c3ccc(-c4ccc5c(c4)CCCC5)cc3)c3ccccc3-c3ccccc3)cc21 ; CC1(C)c2ccccc2-c2ccc(N(c3ccc(-c4ccc5c(c4)CCCC5)cc3)c3ccccc3-c3ccccc3)cc21]

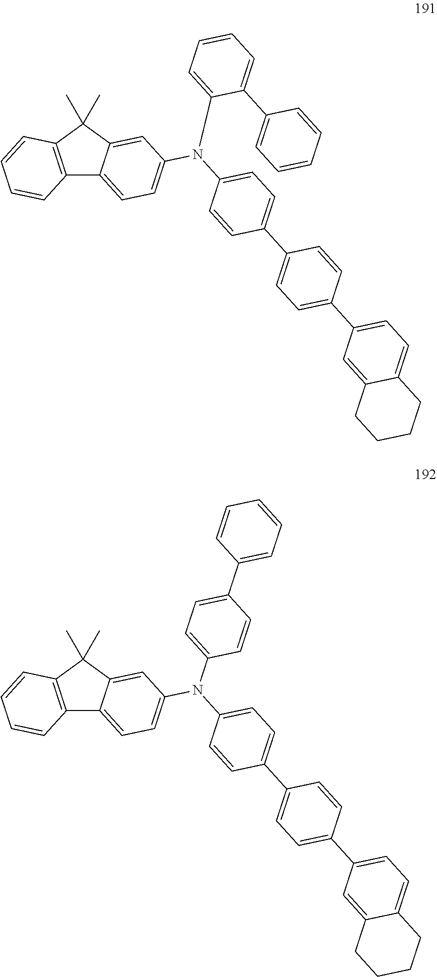 [SMILES: CC1(C)c2ccccc2-c2ccc(N(c3ccc(-c4ccc(-c5ccc6c(c5)CCCC6)cc4)cc3)c3ccccc3-c3ccccc3)cc21 ; CC1(C)c2ccccc2-c2ccc(N(c3ccc(-c4ccccc4)cc3)c3ccc(-c4ccc(-c5ccc6c(c5)CCCC6)cc4)cc3)cc21]

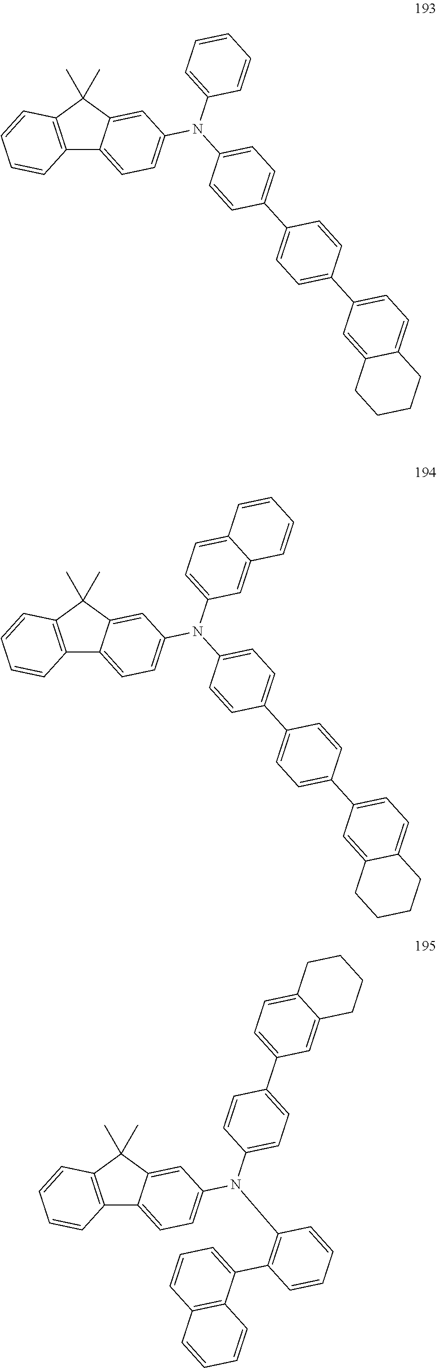 [SMILES: CC1(C)c2ccccc2-c2ccc(N(c3ccccc3)c3ccc(-c4ccc(-c5ccc6c(c5)CCCC6)cc4)cc3)cc21 ; CC1(C)c2ccccc2-c2ccc(N(c3ccc(-c4ccc(-c5ccc6c(c5)CCCC6)cc4)cc3)c3ccc4ccccc4c3)cc21 ; CC1(C)c2ccccc2-c2ccc(N(c3ccc(-c4ccc5c(c4)CCCC5)cc3)c3ccccc3-c3cccc4ccccc34)cc21]

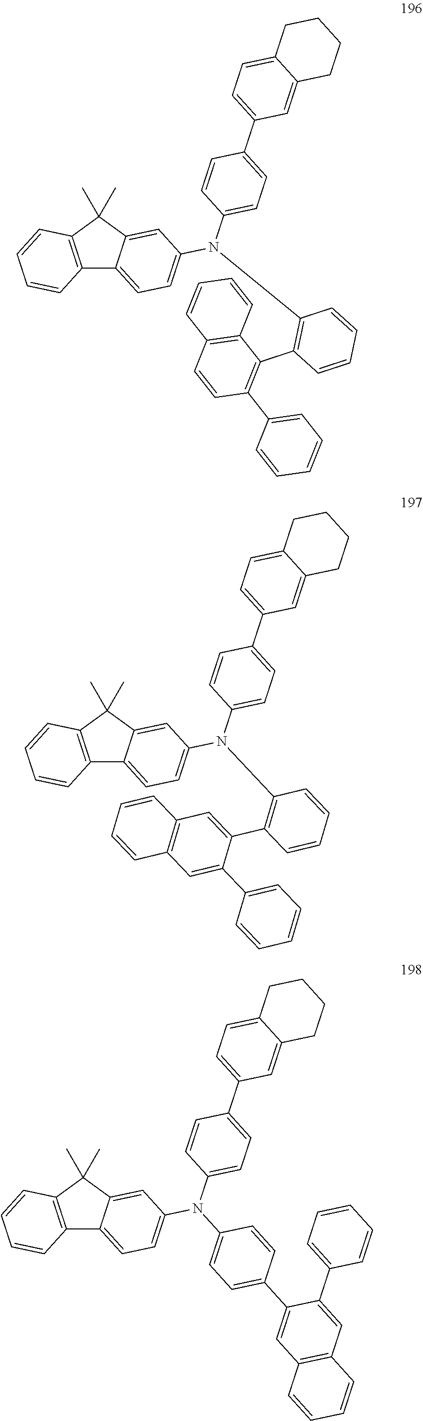

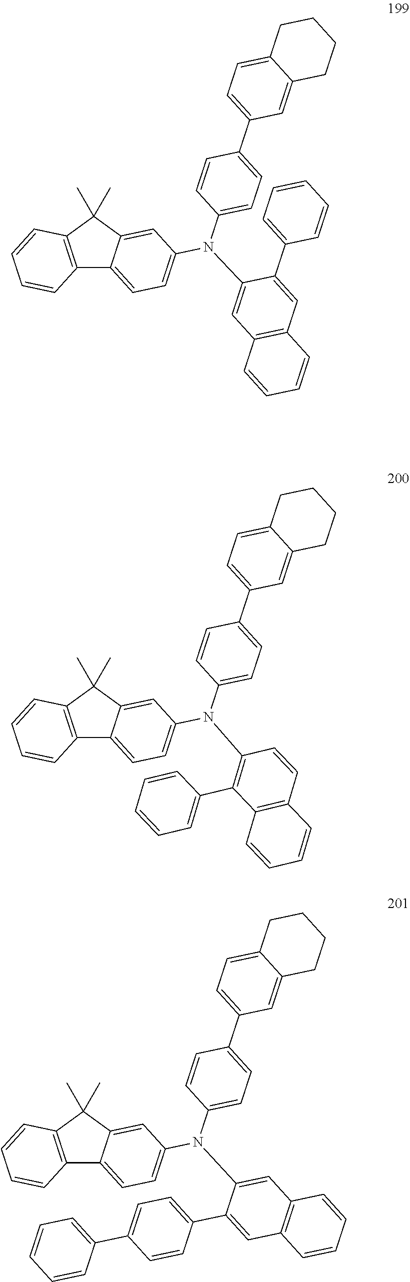 [SMILES: CC1(C)c2ccccc2-c2ccc(N(c3ccc(-c4ccc5c(c4)CCCC5)cc3)c3cc4ccccc4cc3-c3ccccc3)cc21 ; CC1(C)c2ccccc2-c2ccc(N(c3ccc(-c4ccc5c(c4)CCCC5)cc3)c3ccc4ccccc4c3-c3ccccc3)cc21 ; CC1(C)c2ccccc2-c2ccc(N(c3ccc(-c4ccc5c(c4)CCCC5)cc3)c3cc4ccccc4cc3-c3ccc(-c4ccccc4)cc3)cc21]

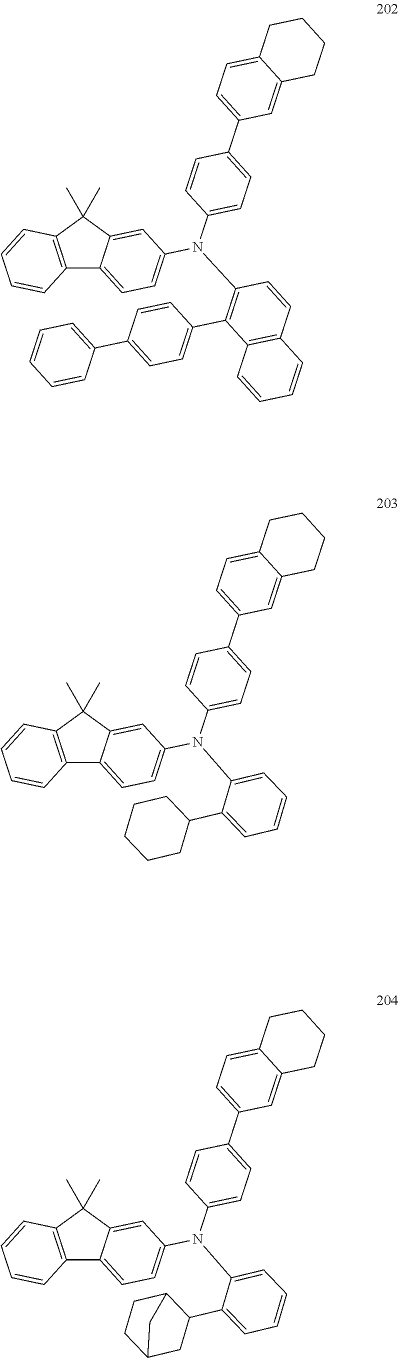 [SMILES: CC1(C)c2ccccc2-c2ccc(N(c3ccc(-c4ccc5c(c4)CCCC5)cc3)c3ccc4ccccc4c3-c3ccc(-c4ccccc4)cc3)cc21 ; CC1(C)c2ccccc2-c2ccc(N(c3ccc(-c4ccc5c(c4)CCCC5)cc3)c3ccccc3C3CCCCC3)cc21 ; CC1(C)c2ccccc2-c2ccc(N(c3ccc(-c4ccc5c(c4)CCCC5)cc3)c3ccccc3C3CC4CCC3C4)cc21]

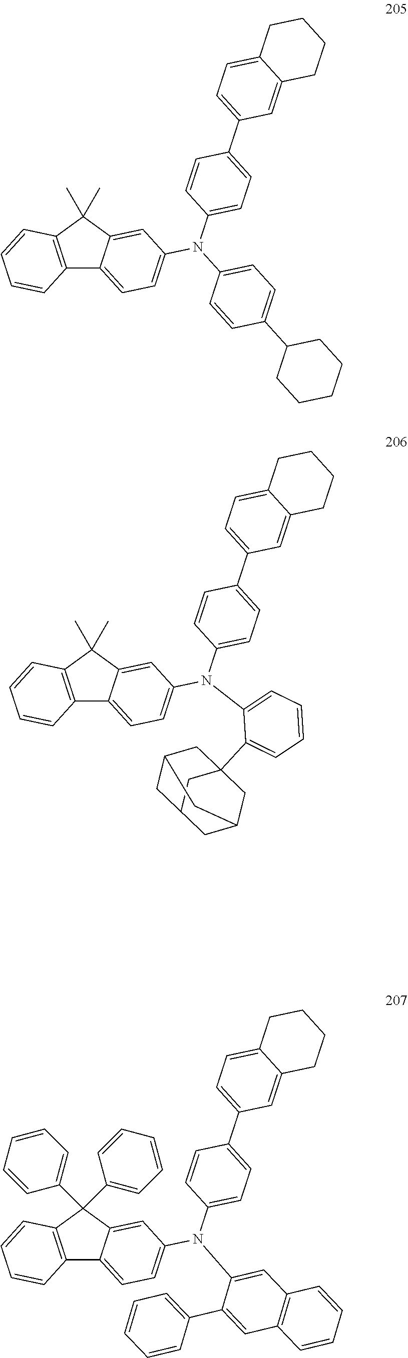 [SMILES: CC1(C)c2ccccc2-c2ccc(N(c3ccc(-c4ccc5c(c4)CCCC5)cc3)c3ccc(C4CCCCC4)cc3)cc21 ; CC1(C)c2ccccc2-c2ccc(N(c3ccc(-c4ccc5c(c4)CCCC5)cc3)c3ccccc3C34CC5CC(CC(C5)C3)C4)cc21 ; c1ccc(-c2cc3ccccc3cc2N(c2ccc(-c3ccc4c(c3)CCCC4)cc2)c2ccc3c(c2)C(c2ccccc2)(c2ccccc2)c2ccccc2-3)cc1]

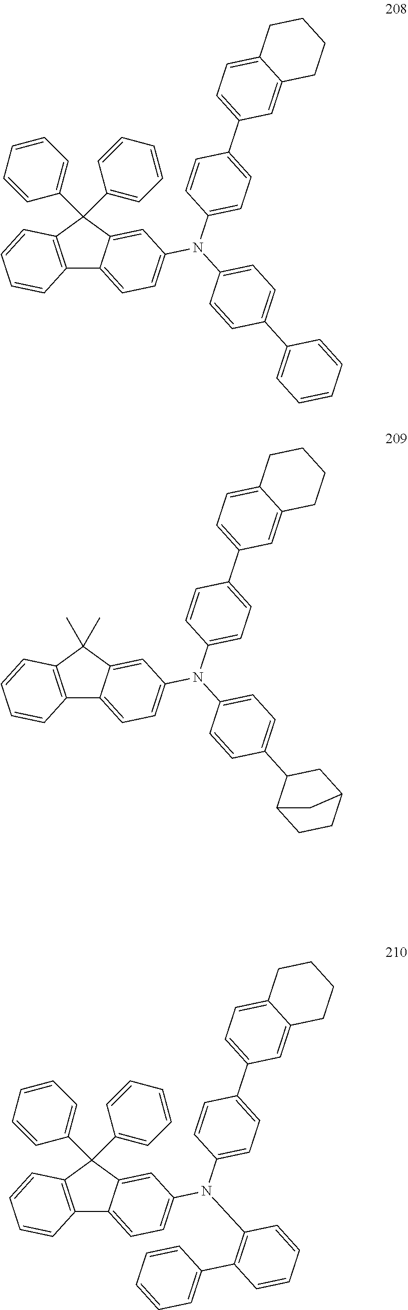

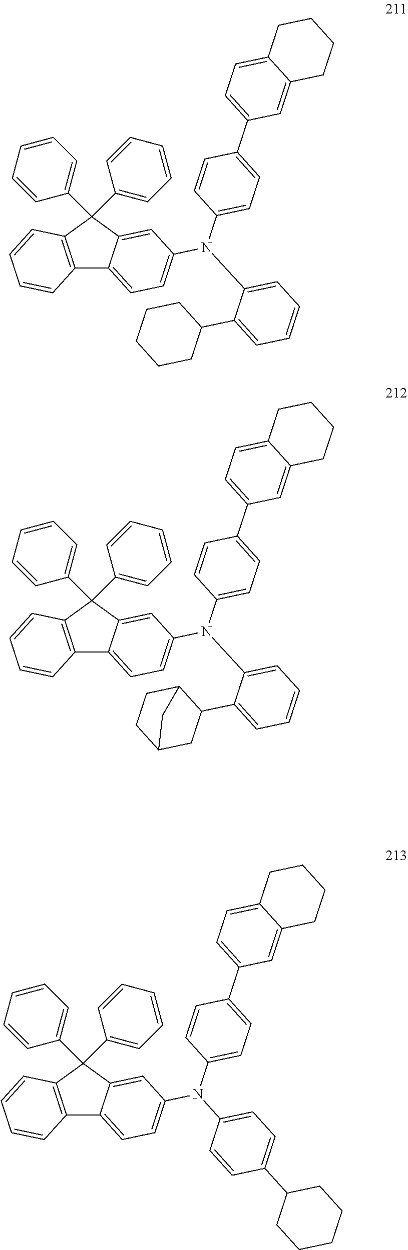 [SMILES: c1ccc(C2(c3ccccc3)c3ccccc3-c3ccc(N(c4ccc(-c5ccc6c(c5)CCCC6)cc4)c4ccccc4C4CCCCC4)cc32)cc1 ; c1ccc(C2(c3ccccc3)c3ccccc3-c3ccc(N(c4ccc(-c5ccc6c(c5)CCCC6)cc4)c4ccccc4C4CC5CCC4C5)cc32)cc1 ; c1ccc(C2(c3ccccc3)c3ccccc3-c3ccc(N(c4ccc(-c5ccc6c(c5)CCCC6)cc4)c4ccc(C5CCCCC5)cc4)cc32)cc1]

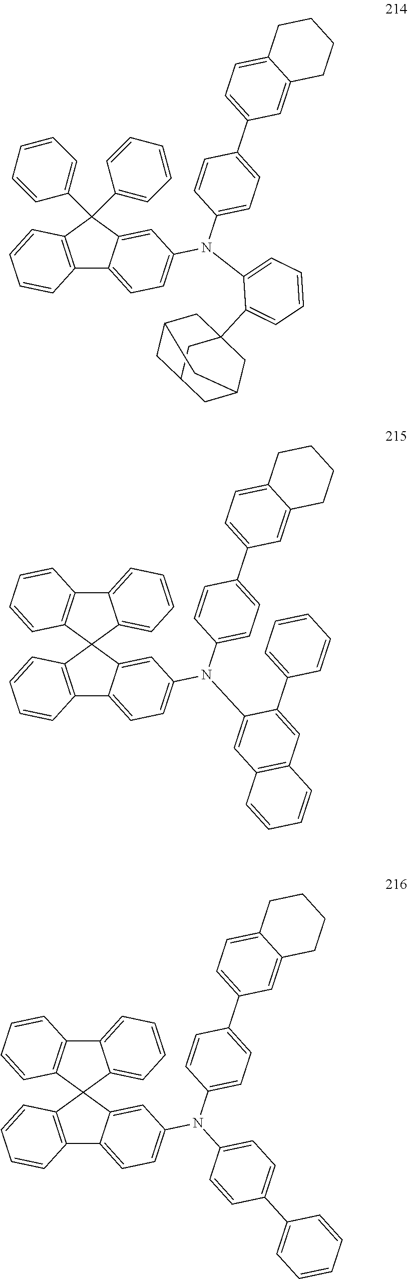 [SMILES: c1ccc(C2(c3ccccc3)c3ccccc3-c3ccc(N(c4ccc(-c5ccc6c(c5)CCCC6)cc4)c4ccccc4C45CC6CC(CC(C6)C4)C5)cc32)cc1 ; c1ccc(-c2cc3ccccc3cc2N(c2ccc(-c3ccc4c(c3)CCCC4)cc2)c2ccc3c(c2)C2(c4ccccc4-c4ccccc42)c2ccccc2-3)cc1 ; c1ccc(-c2ccc(N(c3ccc(-c4ccc5c(c4)CCCC5)cc3)c3ccc4c(c3)C3(c5ccccc5-c5ccccc53)c3ccccc3-4)cc2)cc1]

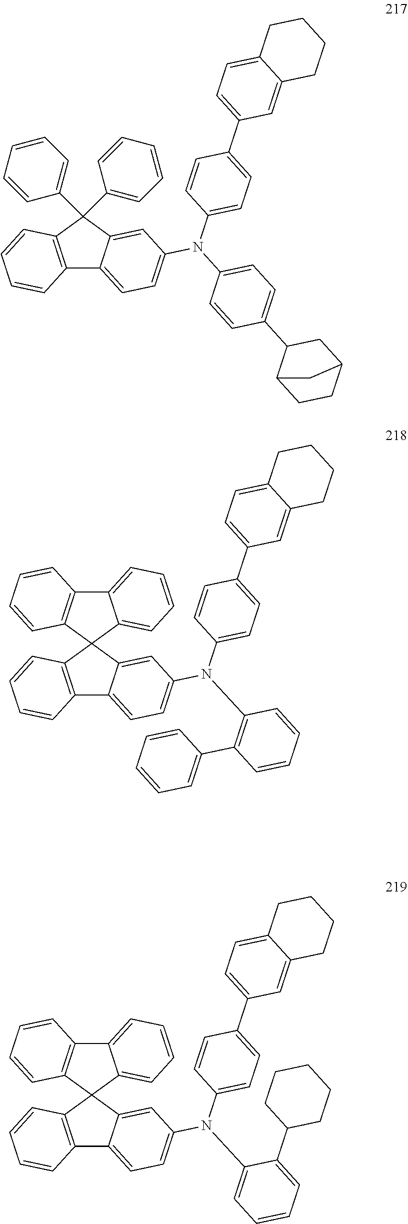

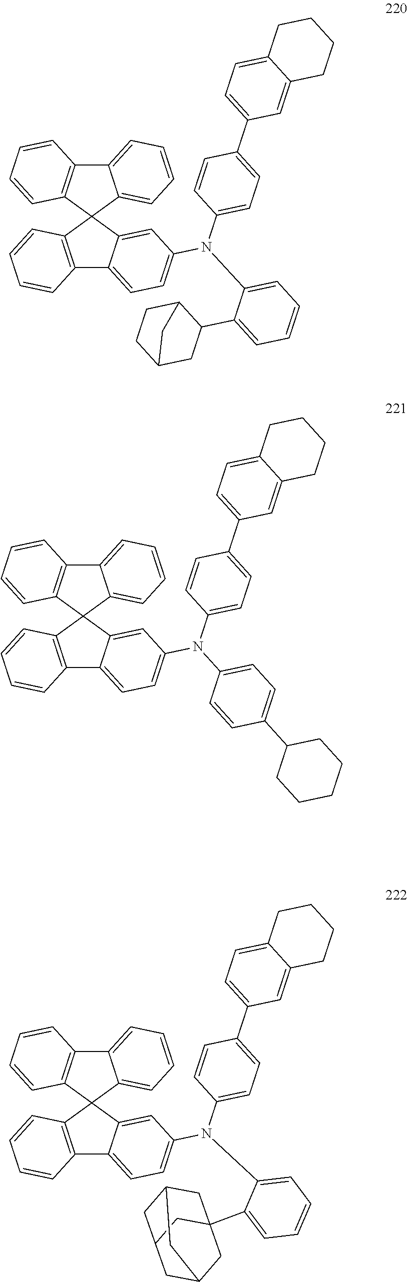 [SMILES: c1ccc2c(c1)-c1ccccc1C21c2ccccc2-c2ccc(N(c3ccc(-c4ccc5c(c4)CCCC5)cc3)c3ccccc3C3CC4CCC3C4)cc21 ; c1ccc2c(c1)-c1ccccc1C21c2ccccc2-c2ccc(N(c3ccc(-c4ccc5c(c4)CCCC5)cc3)c3ccc(C4CCCCC4)cc3)cc21 ; c1ccc2c(c1)-c1ccccc1C21c2ccccc2-c2ccc(N(c3ccc(-c4ccc5c(c4)CCCC5)cc3)c3ccccc3C34CC5CC(CC(C5)C3)C4)cc21]

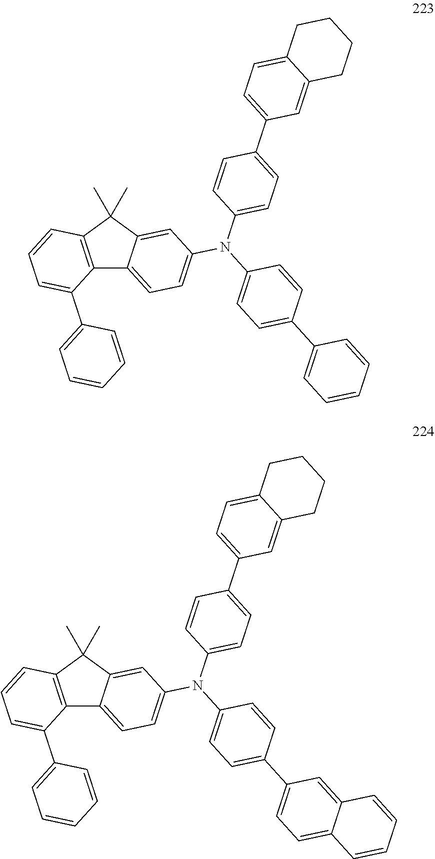 [SMILES: CC1(C)c2cc(N(c3ccc(-c4ccccc4)cc3)c3ccc(-c4ccc5c(c4)CCCC5)cc3)ccc2-c2c(-c3ccccc3)cccc21 ; CC1(C)c2cc(N(c3ccc(-c4ccc5c(c4)CCCC5)cc3)c3ccc(-c4ccc5ccccc5c4)cc3)ccc2-c2c(-c3ccccc3)cccc21]

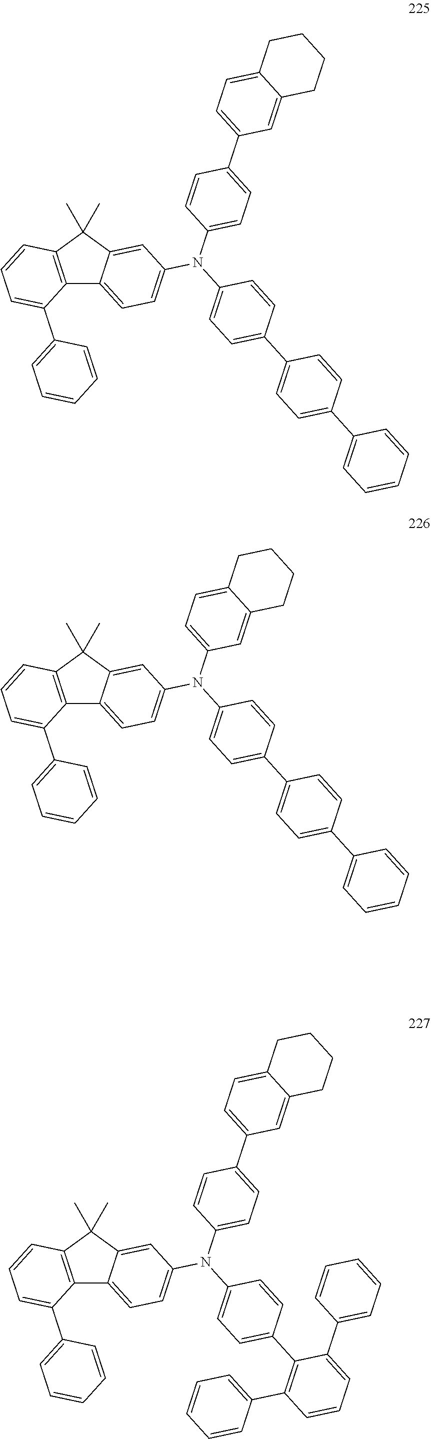 [SMILES: CC1(C)c2cc(N(c3ccc(-c4ccc(-c5ccccc5)cc4)cc3)c3ccc(-c4ccc5c(c4)CCCC5)cc3)ccc2-c2c(-c3ccccc3)cccc21 ; CC1(C)c2cc(N(c3ccc(-c4ccc(-c5ccccc5)cc4)cc3)c3ccc4c(c3)CCCC4)ccc2-c2c(-c3ccccc3)cccc21 ; CC1(C)c2cc(N(c3ccc(-c4ccc5c(c4)CCCC5)cc3)c3ccc(-c4c(-c5ccccc5)cccc4-c4ccccc4)cc3)ccc2-c2c(-c3ccccc3)cccc21]

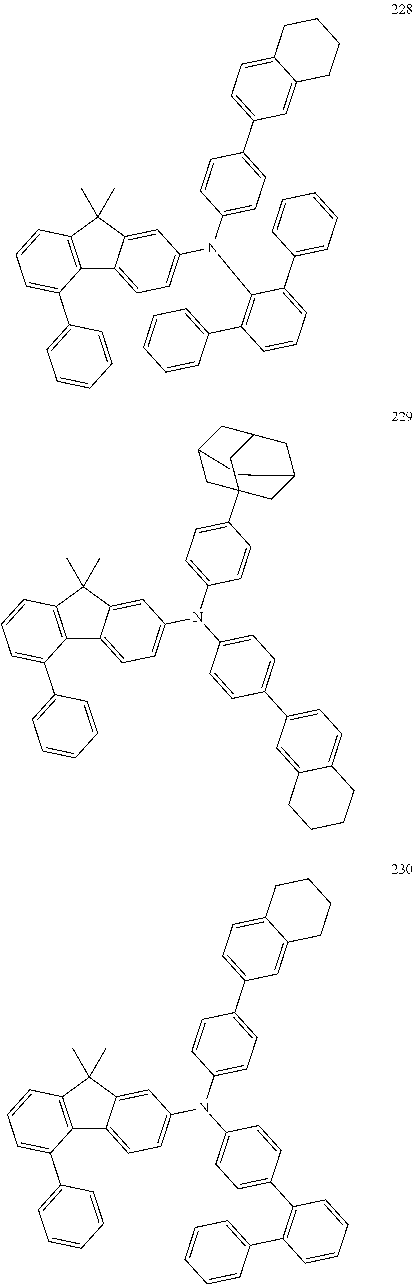 [SMILES: CC1(C)c2cc(N(c3ccc(-c4ccc5c(c4)CCCC5)cc3)c3c(-c4ccccc4)cccc3-c3ccccc3)ccc2-c2c(-c3ccccc3)cccc21 ; CC1(C)c2cc(N(c3ccc(-c4ccc5c(c4)CCCC5)cc3)c3ccc(C45CC6CC(CC(C6)C4)C5)cc3)ccc2-c2c(-c3ccccc3)cccc21 ; CC1(C)c2cc(N(c3ccc(-c4ccc5c(c4)CCCC5)cc3)c3ccc(-c4ccccc4-c4ccccc4)cc3)ccc2-c2c(-c3ccccc3)cccc21]

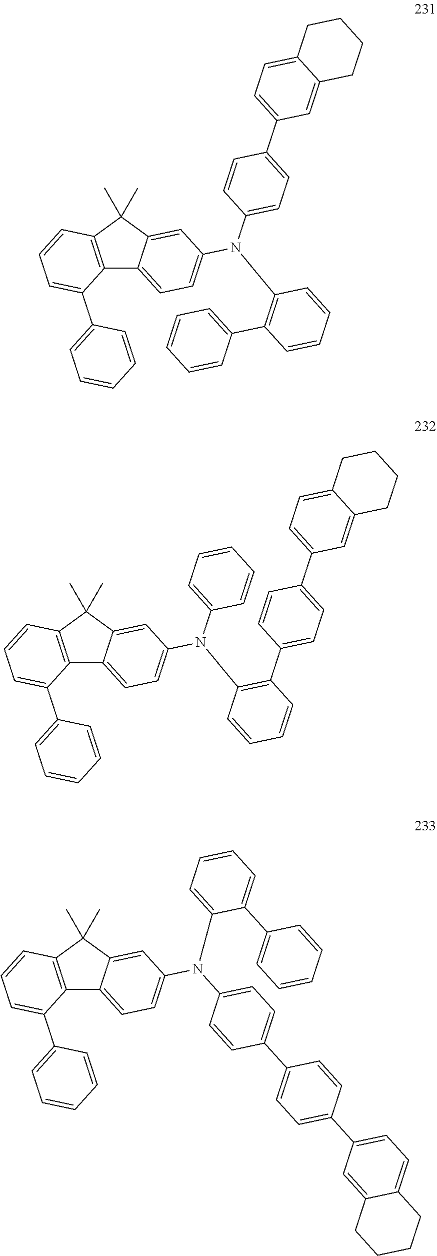 [SMILES: CC1(C)c2cc(N(c3ccc(-c4ccc5c(c4)CCCC5)cc3)c3ccccc3-c3ccccc3)ccc2-c2c(-c3ccccc3)cccc21 ; CC1(C)c2cc(N(c3ccccc3)c3ccccc3-c3ccc(-c4ccc5c(c4)CCCC5)cc3)ccc2-c2c(-c3ccccc3)cccc21 ; CC1(C)c2cc(N(c3ccc(-c4ccc(-c5ccc6c(c5)CCCC6)cc4)cc3)c3ccccc3-c3ccccc3)ccc2-c2c(-c3ccccc3)cccc21]

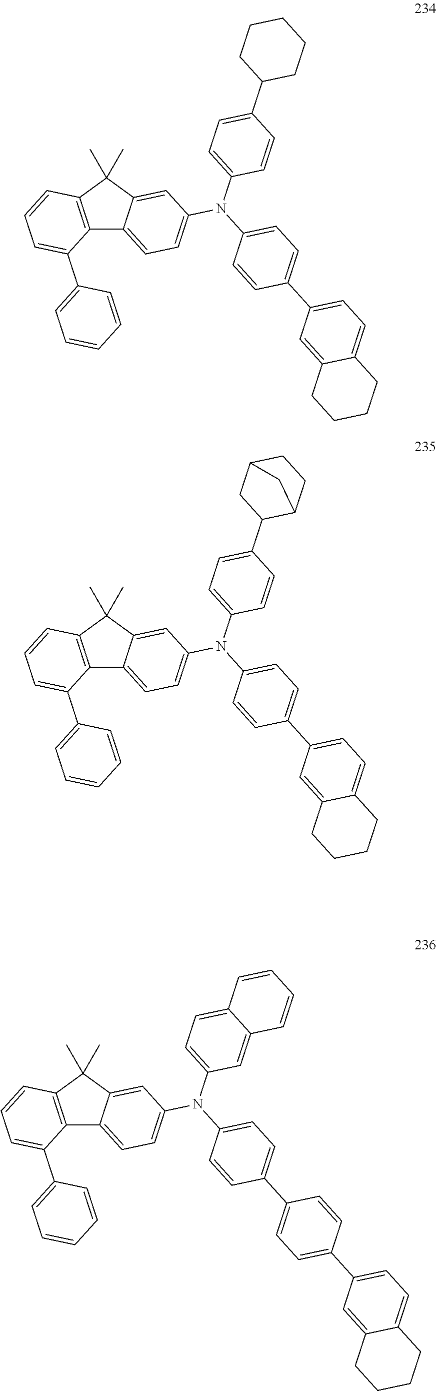 [SMILES: CC1(C)c2cc(N(c3ccc(-c4ccc5c(c4)CCCC5)cc3)c3ccc(C4CCCCC4)cc3)ccc2-c2c(-c3ccccc3)cccc21 ; CC1(C)c2cc(N(c3ccc(-c4ccc5c(c4)CCCC5)cc3)c3ccc(C4CC5CCC4C5)cc3)ccc2-c2c(-c3ccccc3)cccc21 ; CC1(C)c2cc(N(c3ccc(-c4ccc(-c5ccc6c(c5)CCCC6)cc4)cc3)c3ccc4ccccc4c3)ccc2-c2c(-c3ccccc3)cccc21]

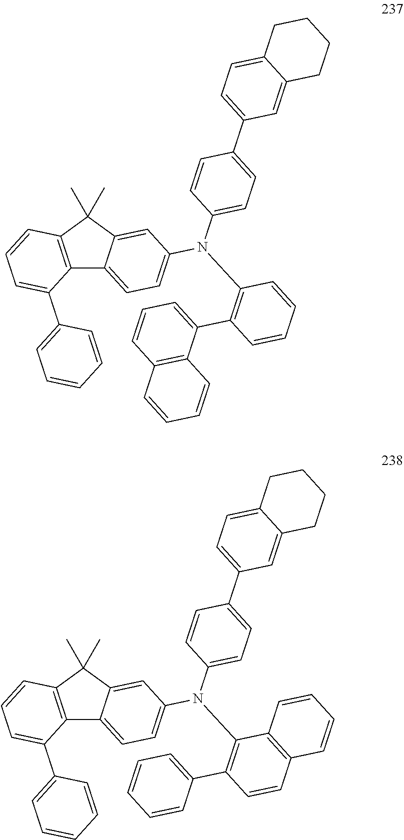 [SMILES: CC1(C)c2cc(N(c3ccc(-c4ccc5c(c4)CCCC5)cc3)c3ccccc3-c3cccc4ccccc34)ccc2-c2c(-c3ccccc3)cccc21 ; CC1(C)c2cc(N(c3ccc(-c4ccc5c(c4)CCCC5)cc3)c3c(-c4ccccc4)ccc4ccccc34)ccc2-c2c(-c3ccccc3)cccc21]

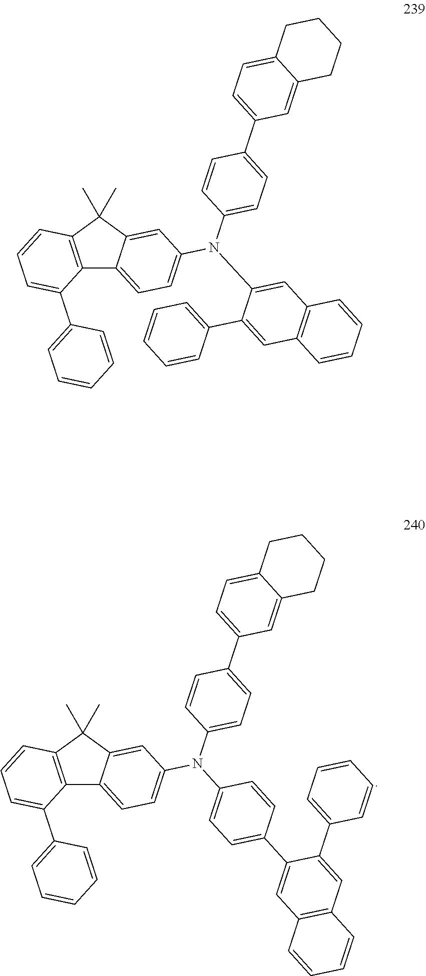 [SMILES: CC1(C)c2cc(N(c3ccc(-c4ccc5c(c4)CCCC5)cc3)c3cc4ccccc4cc3-c3ccccc3)ccc2-c2c(-c3ccccc3)cccc21 ; CC1(C)c2cc(N(c3ccc(-c4ccc5c(c4)CCCC5)cc3)c3ccc(-c4cc5ccccc5cc4-c4ccccc4)cc3)ccc2-c2c(-c3ccccc3)cccc21]

12. An amine compound represented by Formula 1: 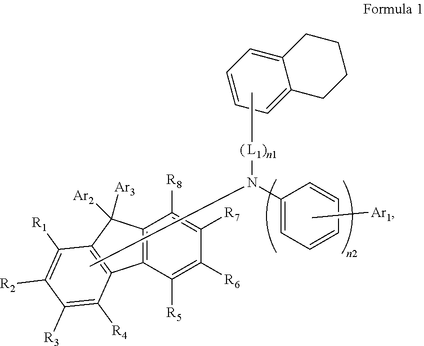
in Formula 1,
n1 is an integer of 0 to 2,
L₁ is a direct linkage, or an unsubstituted arylene group of 6 to 30 ring-forming carbon atoms,
n2 is 0 or 1,
Ar₁ is a substituted or unsubstituted methyl group, a substituted or unsubstituted cyclohexyl group, a substituted or unsubstituted norbornyl group, a substituted or unsubstituted adamantyl group, an unsubstituted phenyl group, an unsubstituted biphenyl group, a substituted or unsubstituted naphthyl group, or a substituted or unsubstituted terphenyl group,
Ar₂ and Ar₃ are each independently a substituted or unsubstituted silyl group, a substituted or unsubstituted alkoxy group of 1 to 30 carbon atoms, a substituted or unsubstituted alkyl group of 1 to 30 carbon atoms, a substituted or unsubstituted alkenyl group of 2 to 30 carbon atoms, a substituted or unsubstituted alkynyl group of 2 to 30 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and
R₁ to R₈ are each independently a hydrogen atom, a deuterium atom, a cyano group, a nitro group, a hydroxyl group, a halogen atom, a substituted or unsubstituted silyl group, a substituted or unsubstituted alkoxy group of 1 to 30 carbon atoms, a substituted or unsubstituted alkyl group of 1 to 30 carbon atoms, a substituted or unsubstituted alkenyl group of 2 to 30 carbon atoms, a substituted or unsubstituted alkynyl group or 2 to 30 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

13. The amine compound of claim 12, wherein, in Formula 1, Ar₁ is represented by any one of Formulae A1-1 to A1-8: 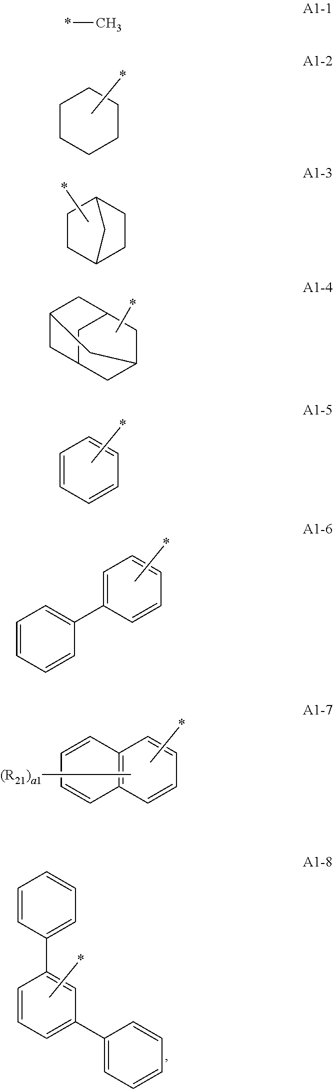
in Formula A1-7,
a1 is an integer of 0 to 7, and
R₂₁ is a hydrogen atom, a deuterium atom, or a substituted or unsubstituted aryl group of 6 to 30 ring-forming carbon atoms.

14. The amine compound of claim 12, wherein Formula 1 is represented by any one of Formula 1-1A to Formula 1-1C: 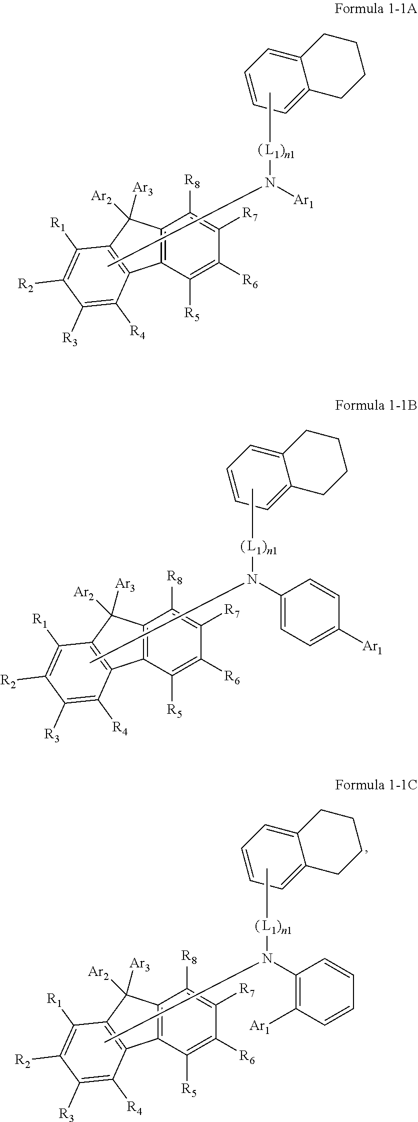
in Formula 1-1A to Formula 1-1C,
Ar₁ to Ar₃, L₁, n1, and R₁ to R₈ are the same as defined in Formula 1.

15. The amine compound of claim 12, wherein Formula 1 is represented by any one of Formula 1-2A to Formula 1-2D: 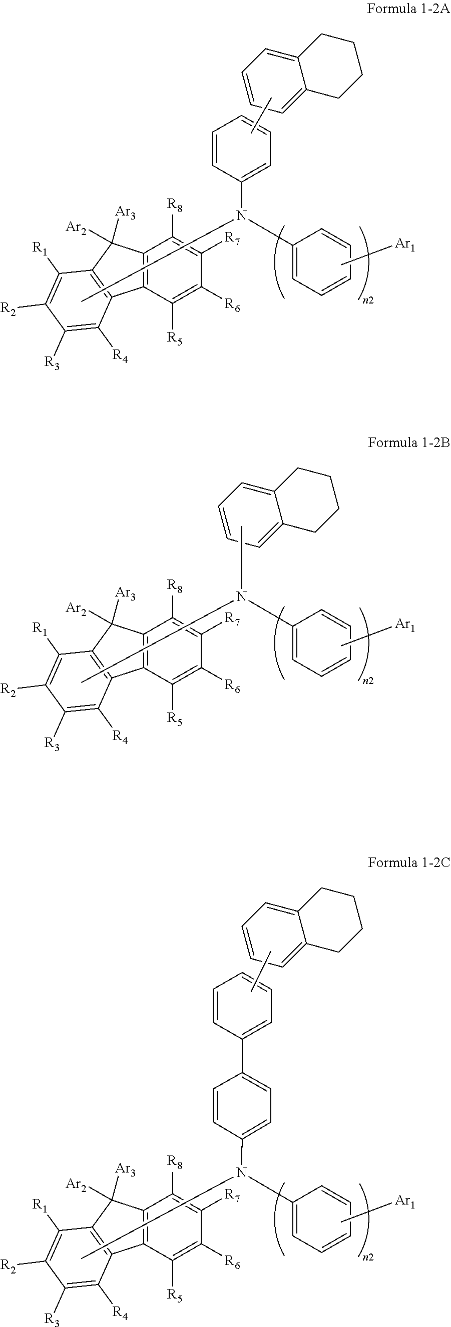 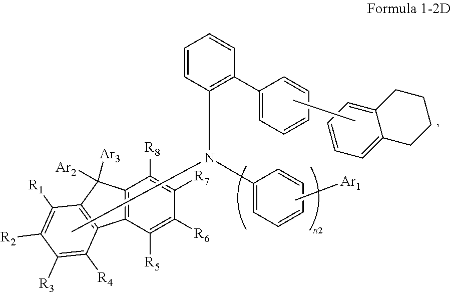
in Formula 1-2A to Formula 1-2D,
Ar₁ to Ar₃, n2, and R₁ to R₈ are the same as defined in Formula 1.

16. The amine compound of claim 15, wherein Formula 1-2A is represented by any one of Formula 1-2AA or Formula 1-2AB: 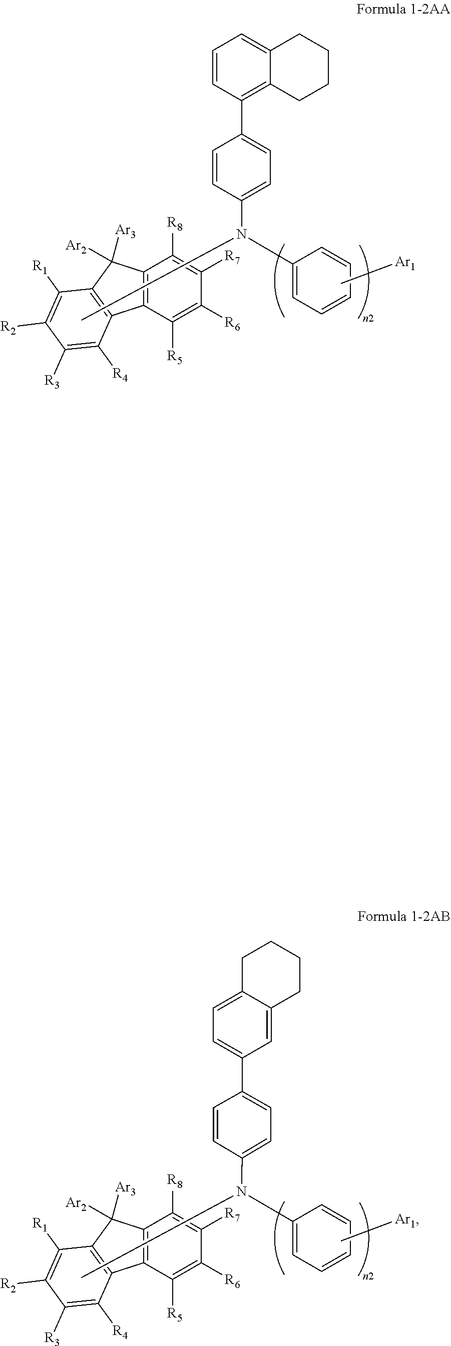
in Formula 1-2AA and Formula 1-2AB,
Ar₁ to Ar₃, n2, and R₁ to R₈ are the same as defined in Formula 1-2A.

17. The amine compound of claim 12, wherein Formula 1 is represented by any one of Formula 1-3A to Formula 1-3C: 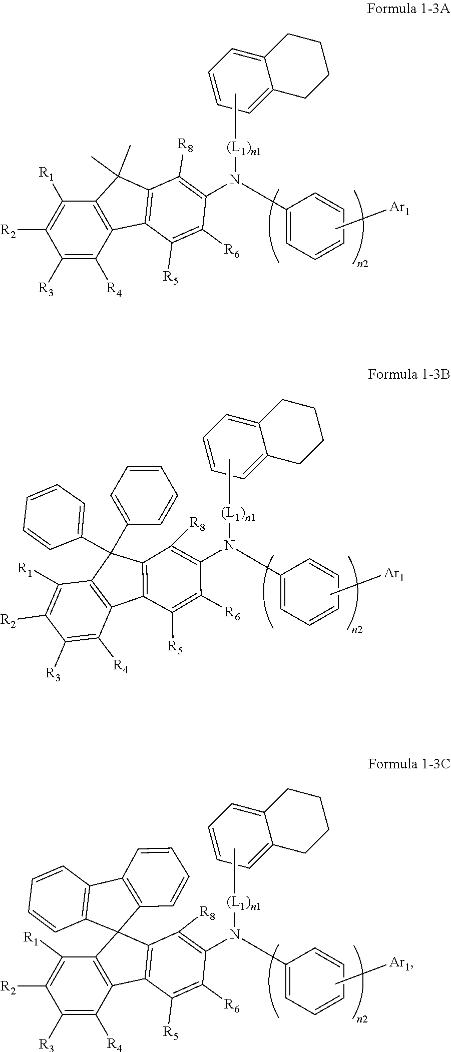
in Formula 1-3A to Formula 1-3C,
Ar₁, L₁, n1, n2, and R₁ to R₈ are the same as defined in Formula 1.

18. The amine compound of claim 17, wherein Formula 1-3A is represented by any one of Formula 1-3AA to Formula 1-3AC: 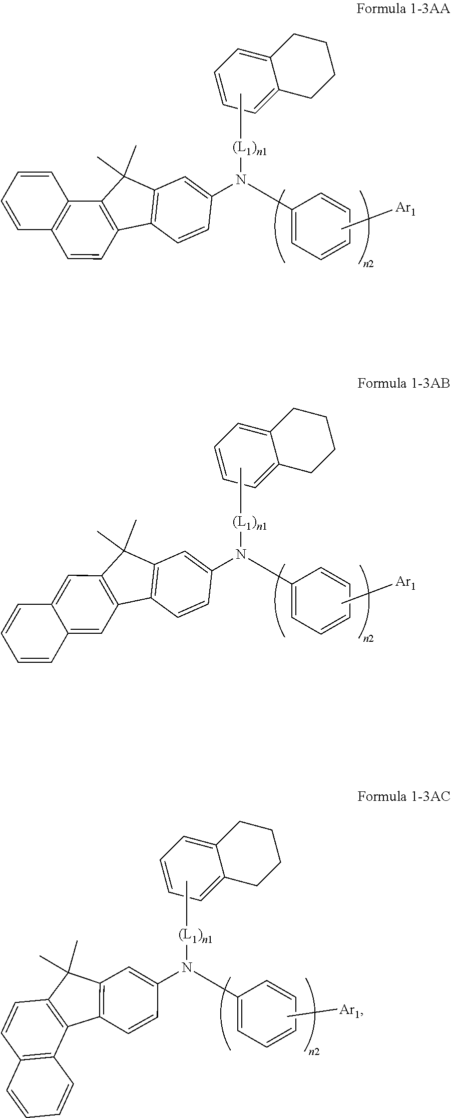
in Formula 1-3AA to Formula 1-3AC,
Ar₁, L₁, n1, and n2 are the same as defined in Formula 1-3A.

19. The amine compound of claim 17, wherein Formula 1-3A is represented by Formula 1-3AD: 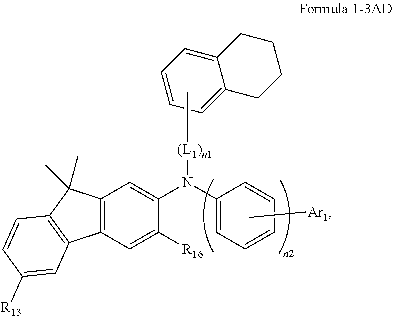
in Formula 1-3AD,
R₁₃ and R₁₆ are each independently a hydrogen atom, a substituted or unsubstituted methyl group, or a substituted or unsubstituted phenyl group, and
Ar₁, L₁, n1 and n2 are the same as defined in Formula 1-3A.

20. The amine compound of claim 12, wherein the amine compound is represented by any one of compounds in Compound Group 1: 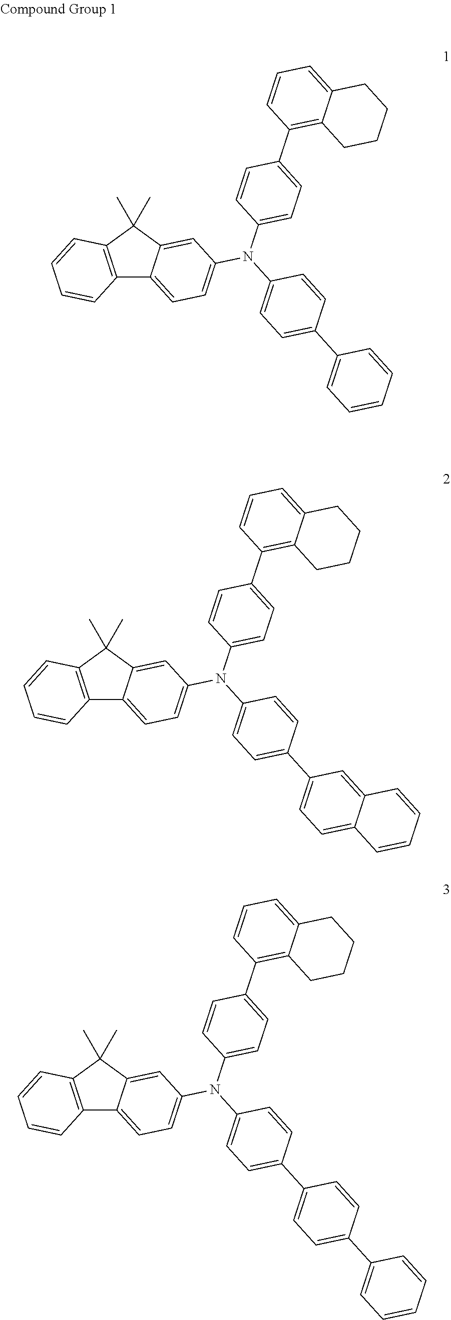 [SMILES: CC1(C)c2ccccc2-c2ccc(N(c3ccc(-c4ccccc4)cc3)c3ccc(-c4cccc5c4CCCC5)cc3)cc21 ; CC1(C)c2ccccc2-c2ccc(N(c3ccc(-c4ccc5ccccc5c4)cc3)c3ccc(-c4cccc5c4CCCC5)cc3)cc21 ; CC1(C)c2ccccc2-c2ccc(N(c3ccc(-c4ccc(-c5ccccc5)cc4)cc3)c3ccc(-c4cccc5c4CCCC5)cc3)cc21] 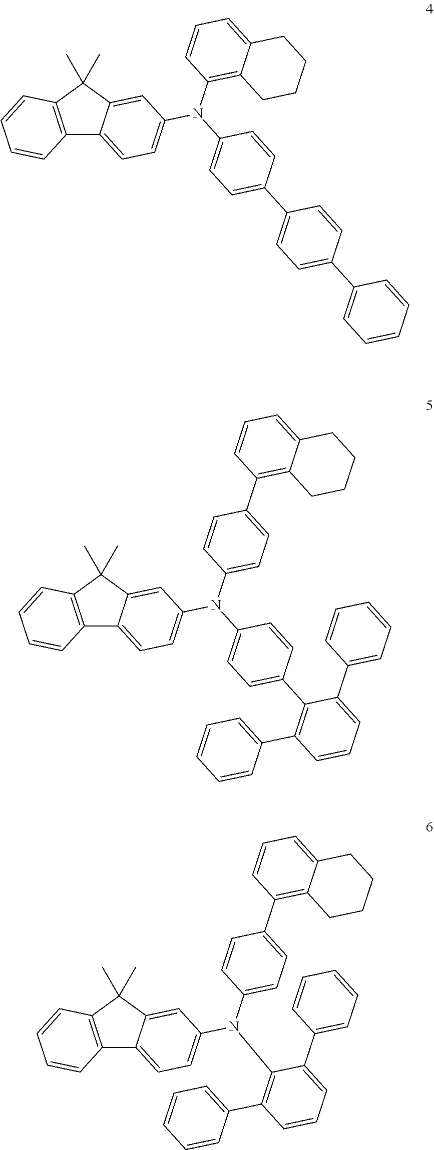 [SMILES: CC1(C)c2ccccc2-c2ccc(N(c3ccc(-c4ccc(-c5ccccc5)cc4)cc3)c3cccc4c3CCCC4)cc21 ; CC1(C)c2ccccc2-c2ccc(N(c3ccc(-c4cccc5c4CCCC5)cc3)c3ccc(-c4c(-c5ccccc5)cccc4-c4ccccc4)cc3)cc21 ; CC1(C)c2ccccc2-c2ccc(N(c3ccc(-c4cccc5c4CCCC5)cc3)c3c(-c4ccccc4)cccc3-c3ccccc3)cc21] 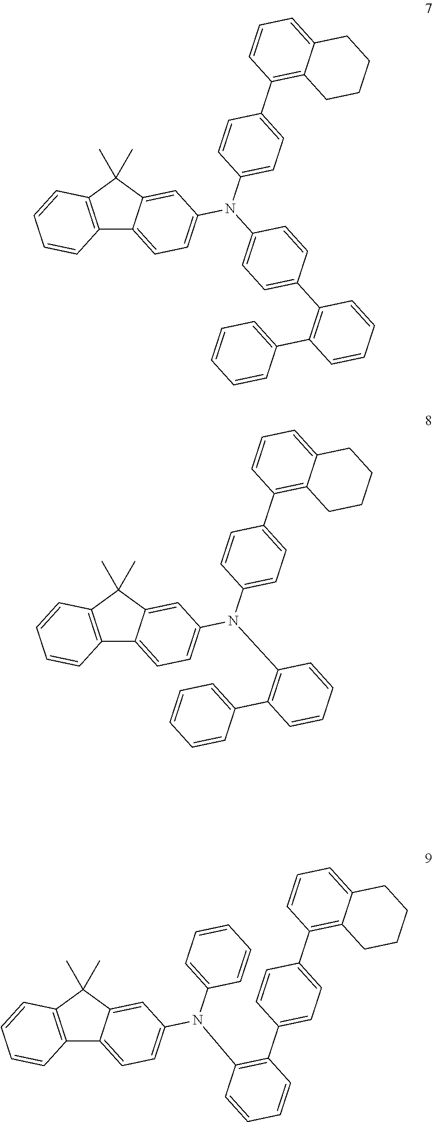 [SMILES: CC1(C)c2ccccc2-c2ccc(N(c3ccc(-c4ccccc4-c4ccccc4)cc3)c3ccc(-c4cccc5c4CCCC5)cc3)cc21 ; CC1(C)c2ccccc2-c2ccc(N(c3ccc(-c4cccc5c4CCCC5)cc3)c3ccccc3-c3ccccc3)cc21 ; CC1(C)c2ccccc2-c2ccc(N(c3ccccc3)c3ccccc3-c3ccc(-c4cccc5c4CCCC5)cc3)cc21] 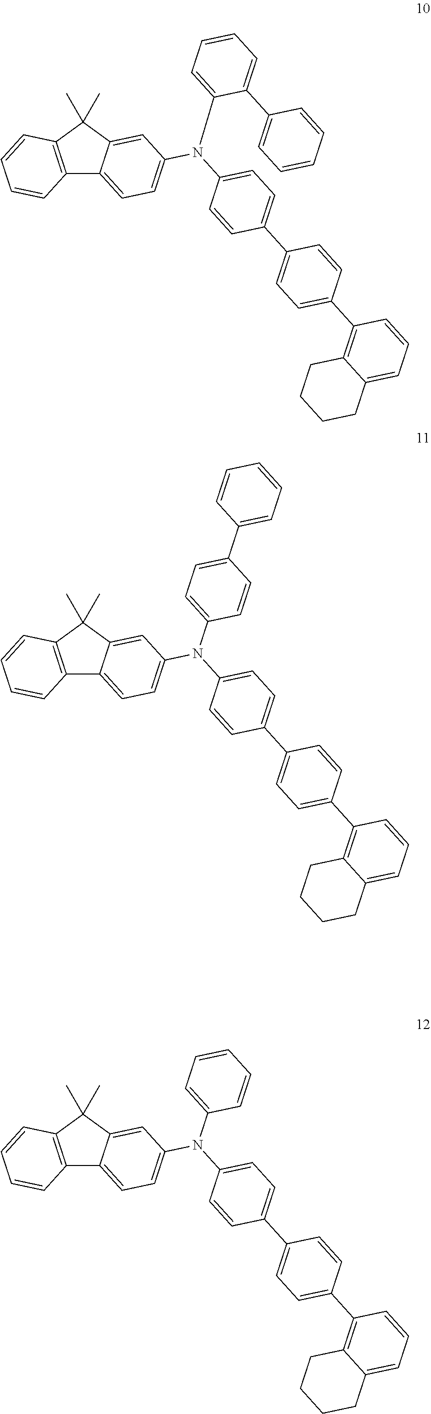 [SMILES: CC1(C)c2ccccc2-c2ccc(N(c3ccc(-c4ccc(-c5cccc6c5CCCC6)cc4)cc3)c3ccccc3-c3ccccc3)cc21 ; CC1(C)c2ccccc2-c2ccc(N(c3ccc(-c4ccccc4)cc3)c3ccc(-c4ccc(-c5cccc6c5CCCC6)cc4)cc3)cc21 ; CC1(C)c2ccccc2-c2ccc(N(c3ccccc3)c3ccc(-c4ccc(-c5cccc6c5CCCC6)cc4)cc3)cc21] 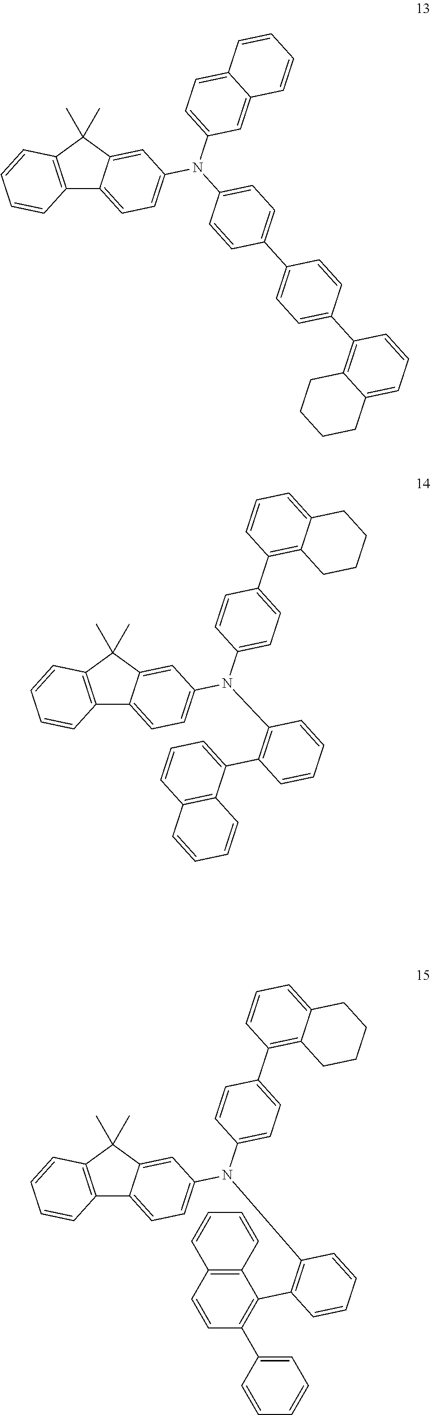 [SMILES: CC1(C)c2ccccc2-c2ccc(N(c3ccc(-c4ccc(-c5cccc6c5CCCC6)cc4)cc3)c3ccc4ccccc4c3)cc21 ; CC1(C)c2ccccc2-c2ccc(N(c3ccc(-c4cccc5c4CCCC5)cc3)c3ccccc3-c3cccc4ccccc34)cc21 ; CC1(C)c2ccccc2-c2ccc(N(c3ccc(-c4cccc5c4CCCC5)cc3)c3ccccc3-c3c(-c4ccccc4)ccc4ccccc34)cc21] 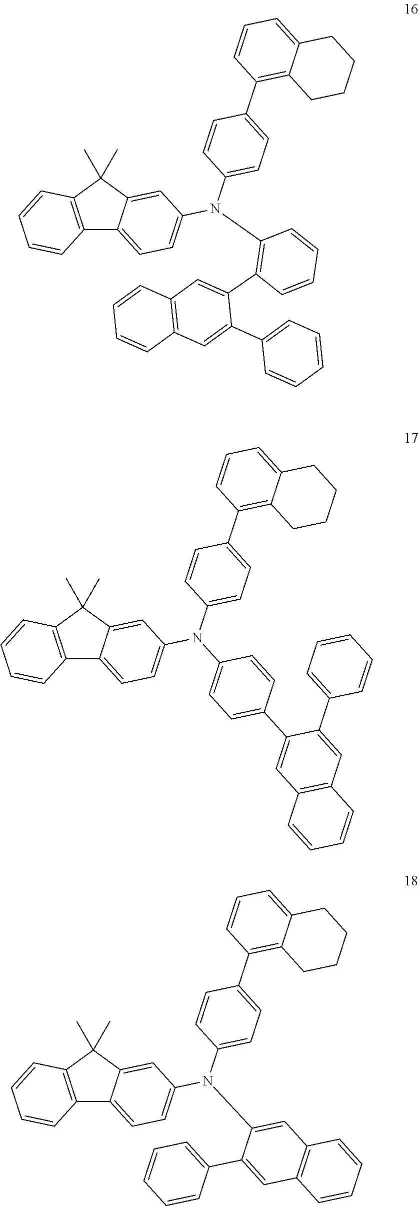 [SMILES: CC1(C)c2ccccc2-c2ccc(N(c3ccc(-c4cccc5c4CCCC5)cc3)c3ccccc3-c3cc4ccccc4cc3-c3ccccc3)cc21 ; CC1(C)c2ccccc2-c2ccc(N(c3ccc(-c4cc5ccccc5cc4-c4ccccc4)cc3)c3ccc(-c4cccc5c4CCCC5)cc3)cc21 ; CC1(C)c2ccccc2-c2ccc(N(c3ccc(-c4cccc5c4CCCC5)cc3)c3cc4ccccc4cc3-c3ccccc3)cc21] 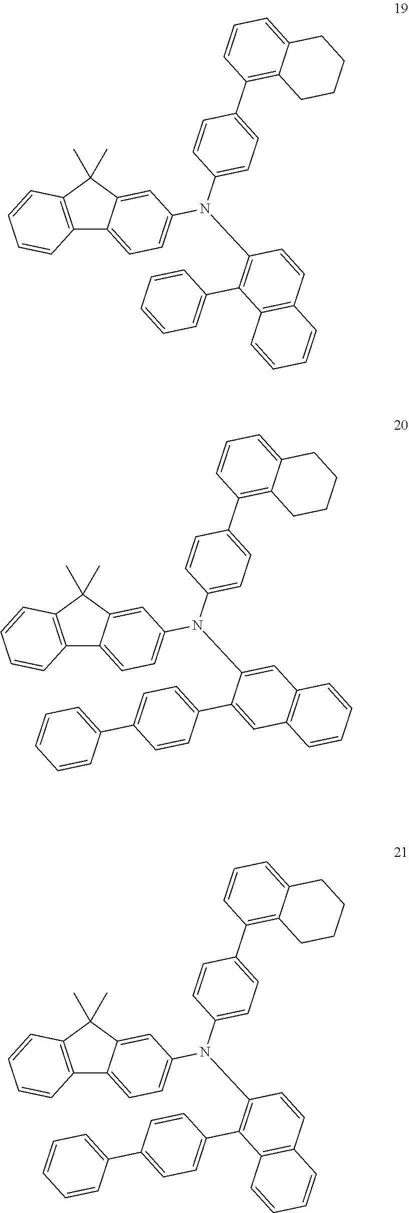 [SMILES: CC1(C)c2ccccc2-c2ccc(N(c3ccc(-c4cccc5c4CCCC5)cc3)c3ccc4ccccc4c3-c3ccccc3)cc21 ; CC1(C)c2ccccc2-c2ccc(N(c3ccc(-c4cccc5c4CCCC5)cc3)c3cc4ccccc4cc3-c3ccc(-c4ccccc4)cc3)cc21 ; CC1(C)c2ccccc2-c2ccc(N(c3ccc(-c4cccc5c4CCCC5)cc3)c3ccc4ccccc4c3-c3ccc(-c4ccccc4)cc3)cc21] 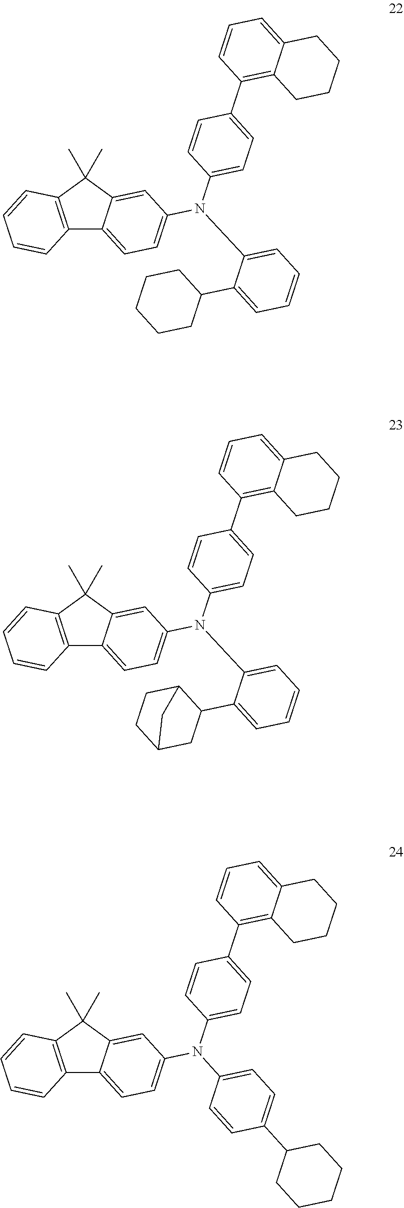 [SMILES: CC1(C)c2ccccc2-c2ccc(N(c3ccc(-c4cccc5c4CCCC5)cc3)c3ccccc3C3CCCCC3)cc21 ; CC1(C)c2ccccc2-c2ccc(N(c3ccc(-c4cccc5c4CCCC5)cc3)c3ccccc3C3CC4CCC3C4)cc21 ; CC1(C)c2ccccc2-c2ccc(N(c3ccc(-c4cccc5c4CCCC5)cc3)c3ccc(C4CCCCC4)cc3)cc21] 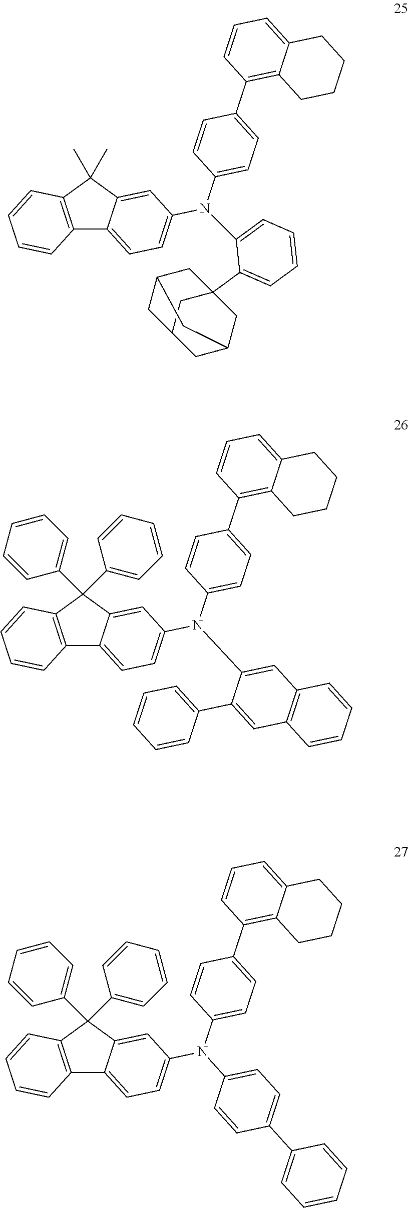 [SMILES: CC1(C)c2ccccc2-c2ccc(N(c3ccc(-c4cccc5c4CCCC5)cc3)c3ccccc3C34CC5CC(CC(C5)C3)C4)cc21 ; c1ccc(-c2cc3ccccc3cc2N(c2ccc(-c3cccc4c3CCCC4)cc2)c2ccc3c(c2)C(c2ccccc2)(c2ccccc2)c2ccccc2-3)cc1 ; c1ccc(-c2ccc(N(c3ccc(-c4cccc5c4CCCC5)cc3)c3ccc4c(c3)C(c3ccccc3)(c3ccccc3)c3ccccc3-4)cc2)cc1] 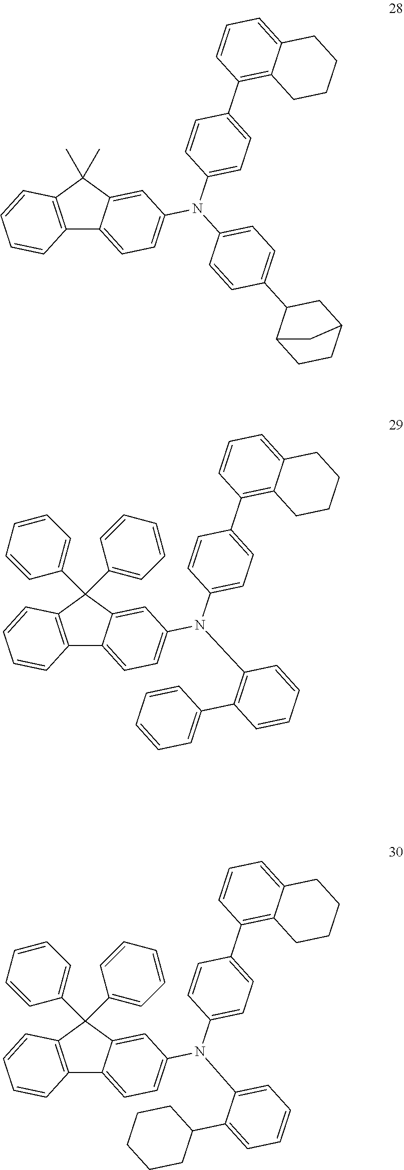 [SMILES: CC1(C)c2ccccc2-c2ccc(N(c3ccc(-c4cccc5c4CCCC5)cc3)c3ccc(C4CC5CCC4C5)cc3)cc21 ; c1ccc(-c2ccccc2N(c2ccc(-c3cccc4c3CCCC4)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5cccc6c5CCCC6)cc4)c4ccccc4C4CCCCC4)cc32)cc1] 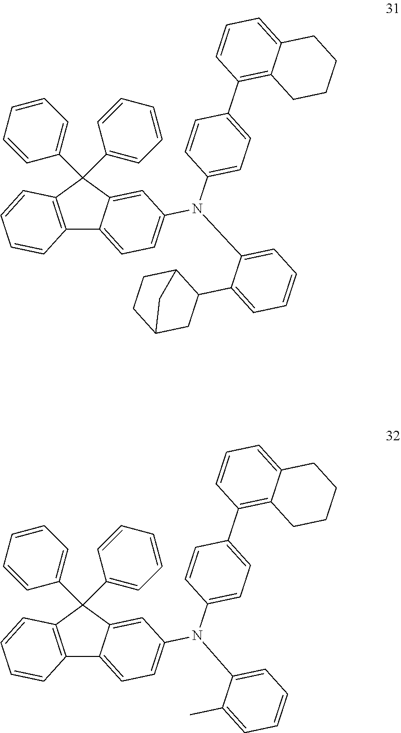 [SMILES: c1ccc(C2(c3ccccc3)c3ccccc3-c3ccc(N(c4ccc(-c5cccc6c5CCCC6)cc4)c4ccccc4C4CC5CCC4C5)cc32)cc1 ; Cc1ccccc1N(c1ccc(-c2cccc3c2CCCC3)cc1)c1ccc2c(c1)C(c1ccccc1)(c1ccccc1)c1ccccc1-2] 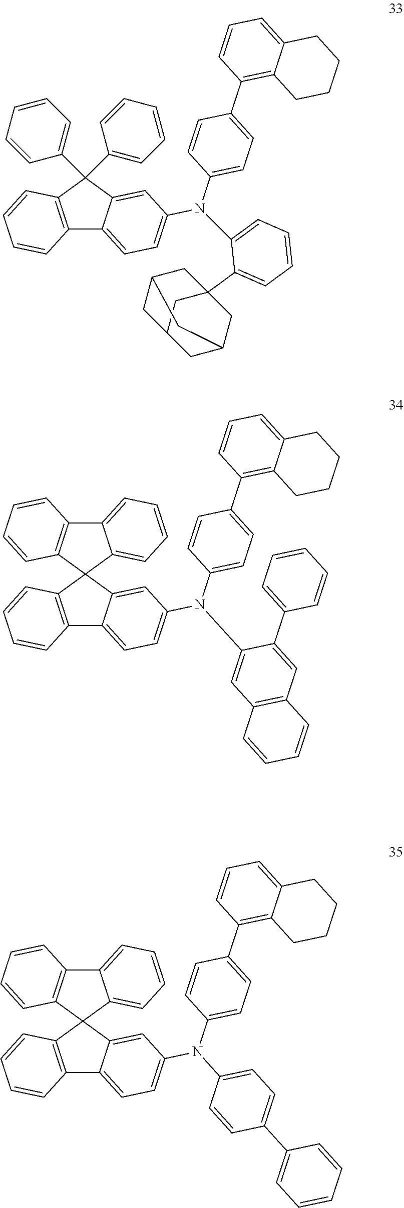 [SMILES: c1ccc(C2(c3ccccc3)c3ccccc3-c3ccc(N(c4ccc(-c5cccc6c5CCCC6)cc4)c4ccccc4C45CC6CC(CC(C6)C4)C5)cc32)cc1 ; c1ccc(-c2cc3ccccc3cc2N(c2ccc(-c3cccc4c3CCCC4)cc2)c2ccc3c(c2)C2(c4ccccc4-c4ccccc42)c2ccccc2-3)cc1 ; c1ccc(-c2ccc(N(c3ccc(-c4cccc5c4CCCC5)cc3)c3ccc4c(c3)C3(c5ccccc5-c5ccccc53)c3ccccc3-4)cc2)cc1] 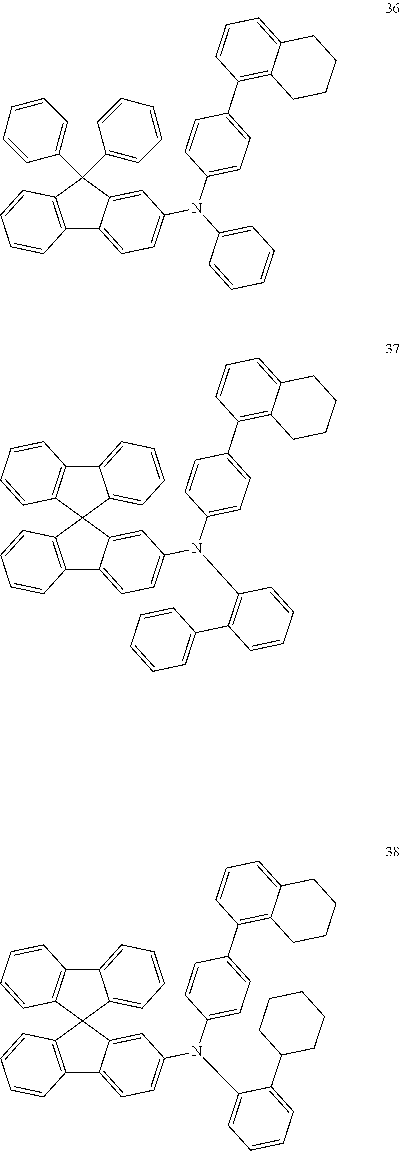 [SMILES: c1ccc(N(c2ccc(-c3cccc4c3CCCC4)cc2)c2ccc3c(c2)C(c2ccccc2)(c2ccccc2)c2ccccc2-3)cc1 ; c1ccc(-c2ccccc2N(c2ccc(-c3cccc4c3CCCC4)cc2)c2ccc3c(c2)C2(c4ccccc4-c4ccccc42)c2ccccc2-3)cc1 ; c1ccc2c(c1)-c1ccccc1C21c2ccccc2-c2ccc(N(c3ccc(-c4cccc5c4CCCC5)cc3)c3ccccc3C3CCCCC3)cc21] 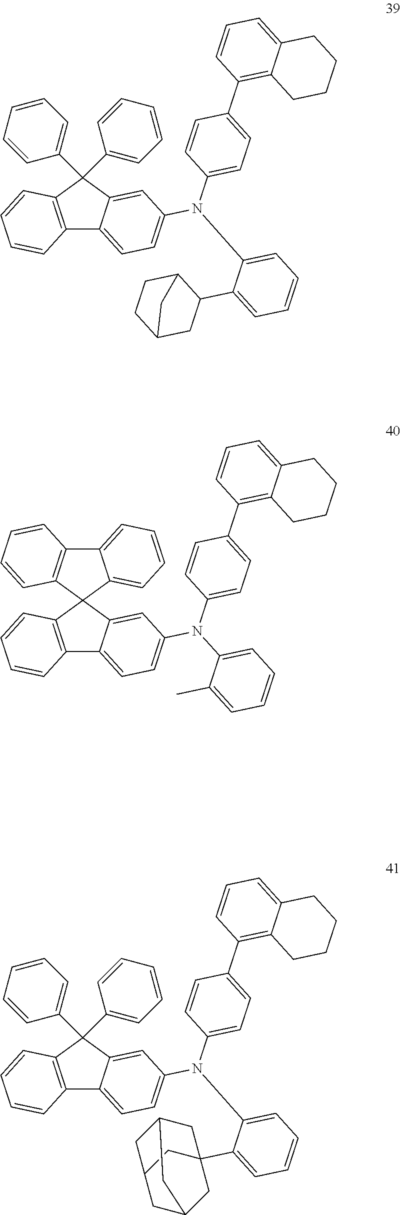 [SMILES: c1ccc(C2(c3ccccc3)c3ccccc3-c3ccc(N(c4ccc(-c5cccc6c5CCCC6)cc4)c4ccccc4C4CC5CCC4C5)cc32)cc1 ; Cc1ccccc1N(c1ccc(-c2cccc3c2CCCC3)cc1)c1ccc2c(c1)C1(c3ccccc3-c3ccccc31)c1ccccc1-2 ; c1ccc(C2(c3ccccc3)c3ccccc3-c3ccc(N(c4ccc(-c5cccc6c5CCCC6)cc4)c4ccccc4C45CC6CC(CC(C6)C4)C5)cc32)cc1] 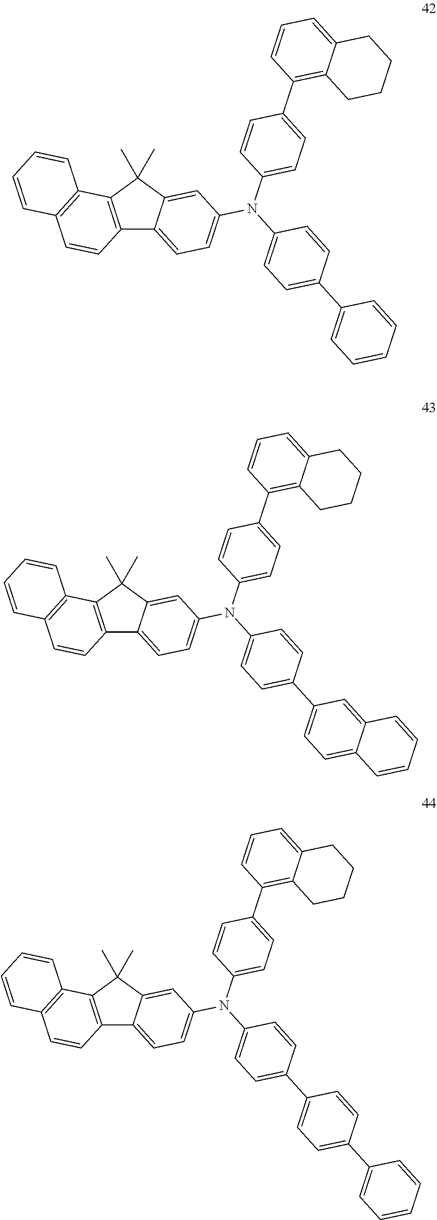 [SMILES: CC1(C)c2cc(N(c3ccc(-c4ccccc4)cc3)c3ccc(-c4cccc5c4CCCC5)cc3)ccc2-c2ccc3ccccc3c21 ; CC1(C)c2cc(N(c3ccc(-c4ccc5ccccc5c4)cc3)c3ccc(-c4cccc5c4CCCC5)cc3)ccc2-c2ccc3ccccc3c21 ; CC1(C)c2cc(N(c3ccc(-c4ccc(-c5ccccc5)cc4)cc3)c3ccc(-c4cccc5c4CCCC5)cc3)ccc2-c2ccc3ccccc3c21] 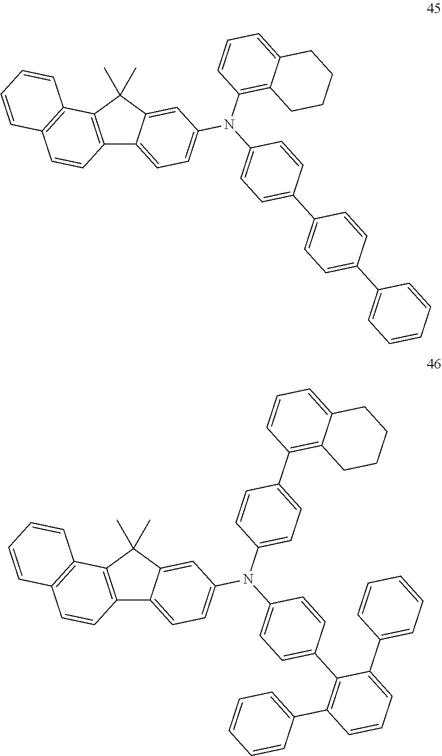 [SMILES: CC1(C)c2cc(N(c3ccc(-c4ccc(-c5ccccc5)cc4)cc3)c3cccc4c3CCCC4)ccc2-c2ccc3ccccc3c21 ; CC1(C)c2cc(N(c3ccc(-c4cccc5c4CCCC5)cc3)c3ccc(-c4c(-c5ccccc5)cccc4-c4ccccc4)cc3)ccc2-c2ccc3ccccc3c21] 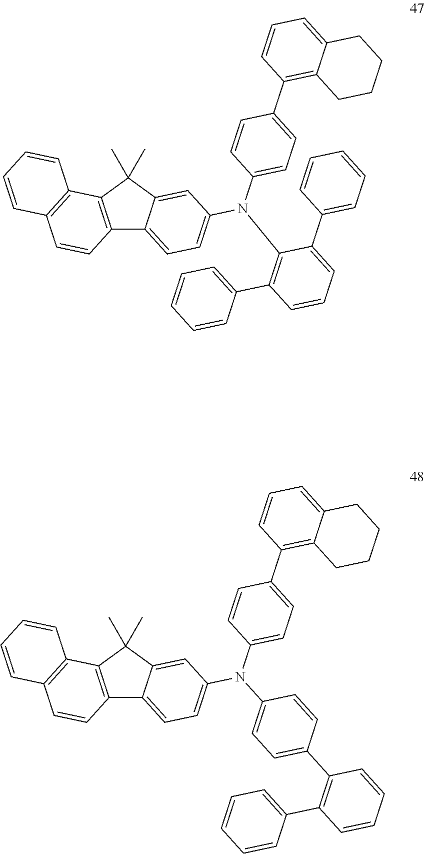 [SMILES: CC1(C)c2cc(N(c3ccc(-c4cccc5c4CCCC5)cc3)c3c(-c4ccccc4)cccc3-c3ccccc3)ccc2-c2ccc3ccccc3c21 ; CC1(C)c2cc(N(c3ccc(-c4ccccc4-c4ccccc4)cc3)c3ccc(-c4cccc5c4CCCC5)cc3)ccc2-c2ccc3ccccc3c21] 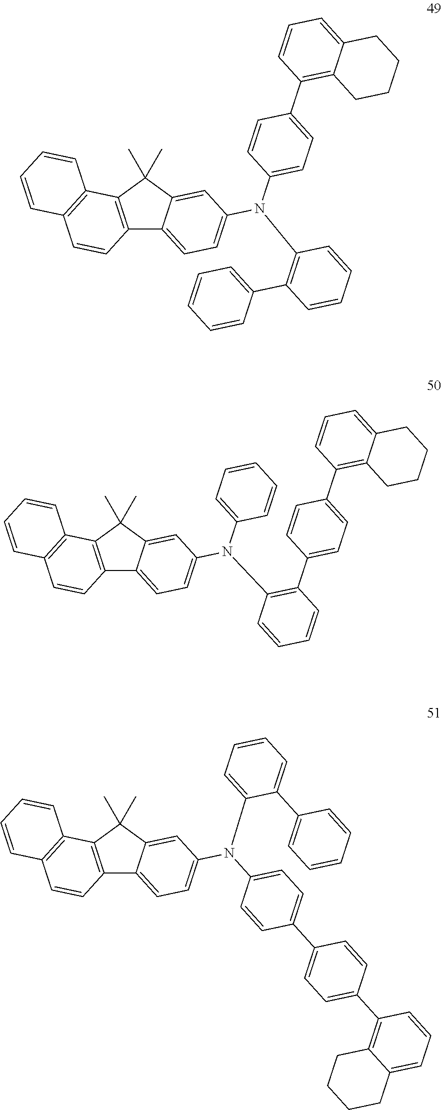 [SMILES: CC1(C)c2cc(N(c3ccc(-c4cccc5c4CCCC5)cc3)c3ccccc3-c3ccccc3)ccc2-c2ccc3ccccc3c21 ; CC1(C)c2cc(N(c3ccccc3)c3ccccc3-c3ccc(-c4cccc5c4CCCC5)cc3)ccc2-c2ccc3ccccc3c21 ; CC1(C)c2cc(N(c3ccc(-c4ccc(-c5cccc6c5CCCC6)cc4)cc3)c3ccccc3-c3ccccc3)ccc2-c2ccc3ccccc3c21] 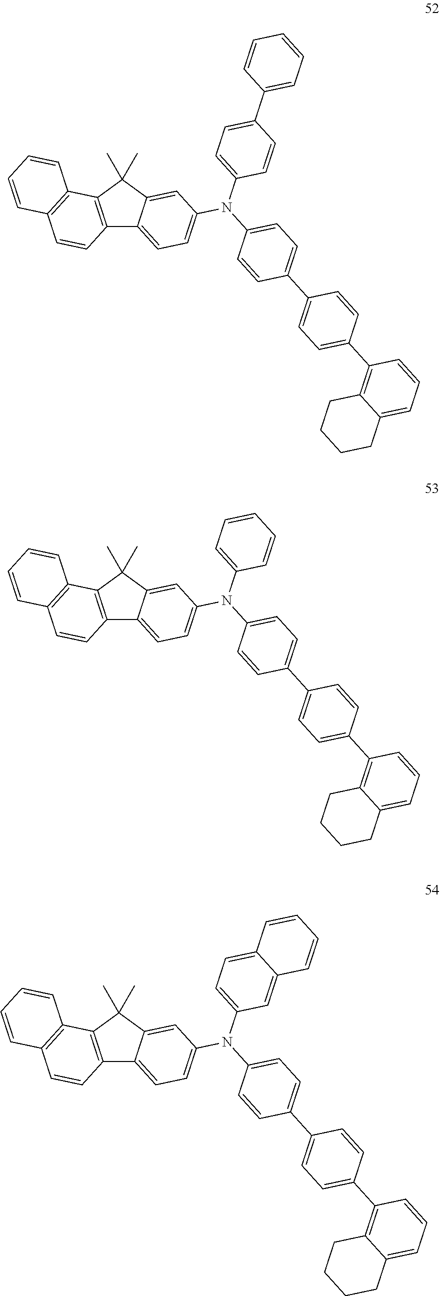 [SMILES: CC1(C)c2cc(N(c3ccc(-c4ccccc4)cc3)c3ccc(-c4ccc(-c5cccc6c5CCCC6)cc4)cc3)ccc2-c2ccc3ccccc3c21 ; CC1(C)c2cc(N(c3ccccc3)c3ccc(-c4ccc(-c5cccc6c5CCCC6)cc4)cc3)ccc2-c2ccc3ccccc3c21 ; CC1(C)c2cc(N(c3ccc(-c4ccc(-c5cccc6c5CCCC6)cc4)cc3)c3ccc4ccccc4c3)ccc2-c2ccc3ccccc3c21] 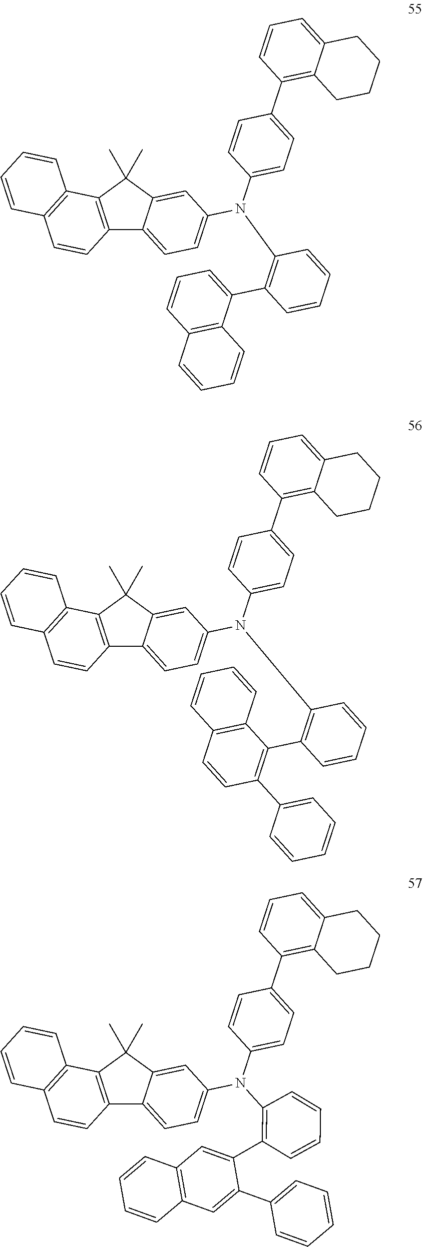 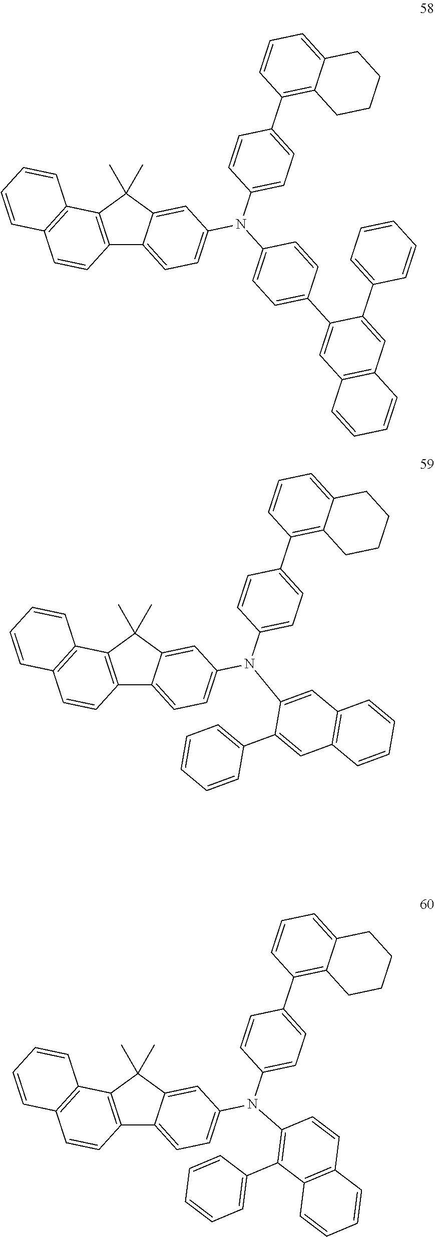 [SMILES: CC1(C)c2cc(N(c3ccc(-c4cc5ccccc5cc4-c4ccccc4)cc3)c3ccc(-c4cccc5c4CCCC5)cc3)ccc2-c2ccc3ccccc3c21 ; CC1(C)c2cc(N(c3ccc(-c4cccc5c4CCCC5)cc3)c3cc4ccccc4cc3-c3ccccc3)ccc2-c2ccc3ccccc3c21 ; CC1(C)c2cc(N(c3ccc(-c4cccc5c4CCCC5)cc3)c3ccc4ccccc4c3-c3ccccc3)ccc2-c2ccc3ccccc3c21] 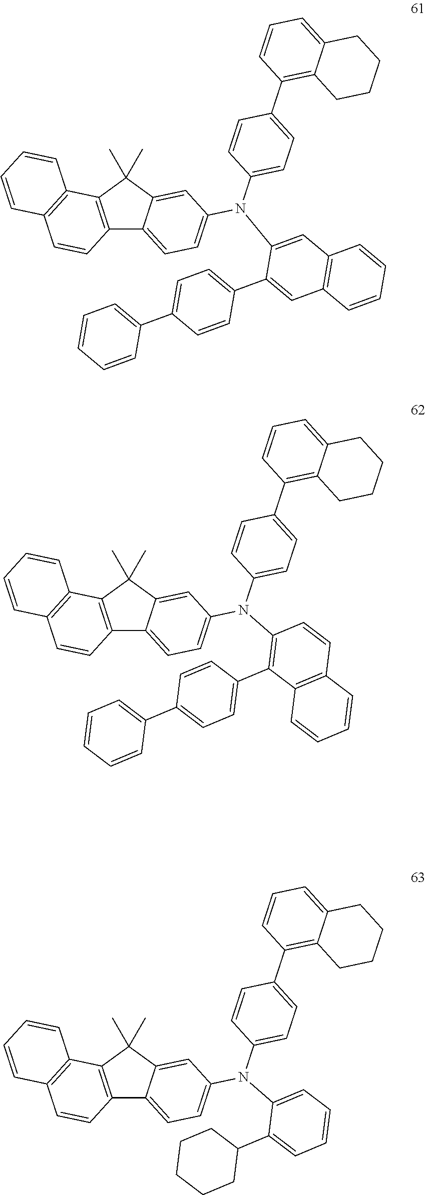 [SMILES: CC1(C)c2cc(N(c3ccc(-c4cccc5c4CCCC5)cc3)c3cc4ccccc4cc3-c3ccc(-c4ccccc4)cc3)ccc2-c2ccc3ccccc3c21 ; CC1(C)c2cc(N(c3ccc(-c4cccc5c4CCCC5)cc3)c3ccc4ccccc4c3-c3ccc(-c4ccccc4)cc3)ccc2-c2ccc3ccccc3c21 ; CC1(C)c2cc(N(c3ccc(-c4cccc5c4CCCC5)cc3)c3ccccc3C3CCCCC3)ccc2-c2ccc3ccccc3c21] 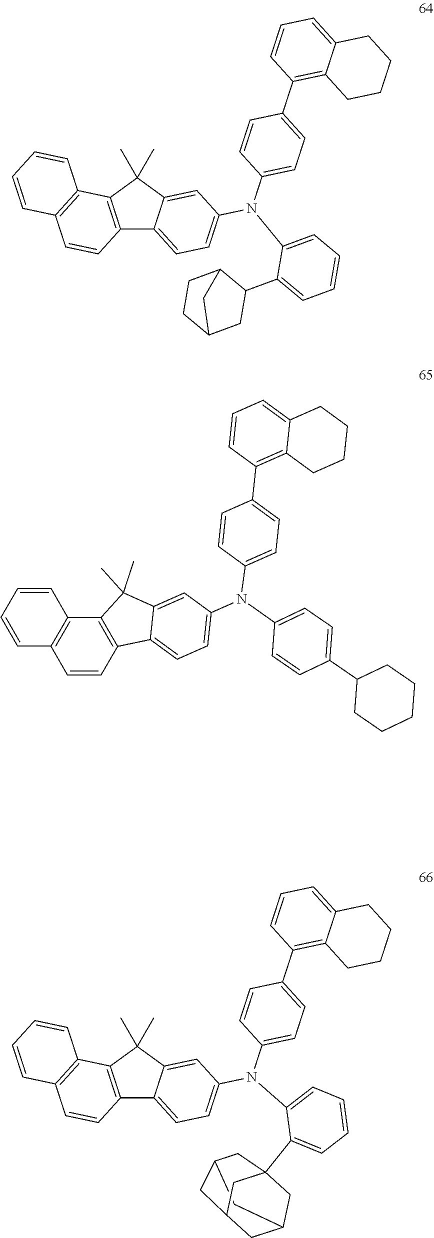 [SMILES: CC1(C)c2cc(N(c3ccc(-c4cccc5c4CCCC5)cc3)c3ccccc3C3CC4CCC3C4)ccc2-c2ccc3ccccc3c21 ; CC1(C)c2cc(N(c3ccc(-c4cccc5c4CCCC5)cc3)c3ccc(C4CCCCC4)cc3)ccc2-c2ccc3ccccc3c21 ; CC1(C)c2cc(N(c3ccc(-c4cccc5c4CCCC5)cc3)c3ccccc3C34CC5CC(CC(C5)C3)C4)ccc2-c2ccc3ccccc3c21] 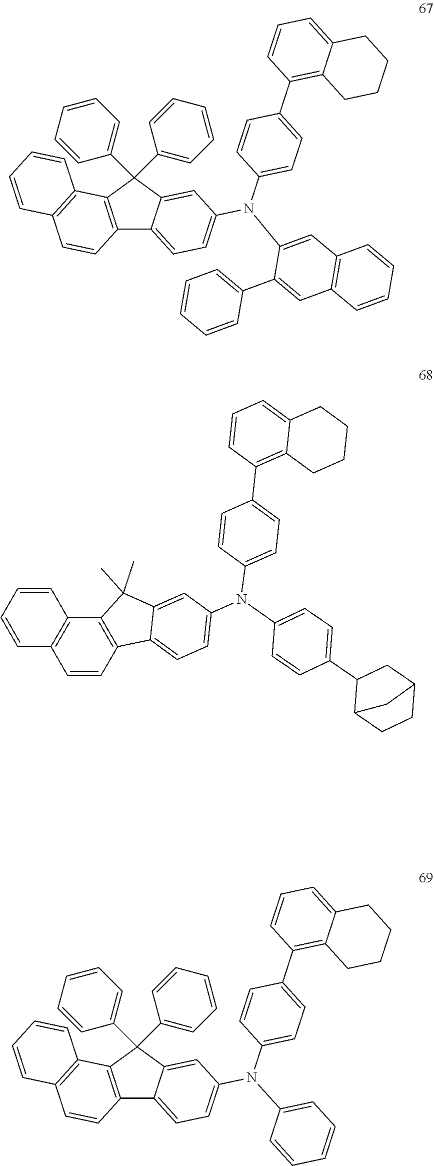 [SMILES: c1ccc(-c2cc3ccccc3cc2N(c2ccc(-c3cccc4c3CCCC4)cc2)c2ccc3c(c2)C(c2ccccc2)(c2ccccc2)c2c-3ccc3ccccc23)cc1 ; CC1(C)c2cc(N(c3ccc(-c4cccc5c4CCCC5)cc3)c3ccc(C4CC5CCC4C5)cc3)ccc2-c2ccc3ccccc3c21 ; c1ccc(N(c2ccc(-c3cccc4c3CCCC4)cc2)c2ccc3c(c2)C(c2ccccc2)(c2ccccc2)c2c-3ccc3ccccc23)cc1] 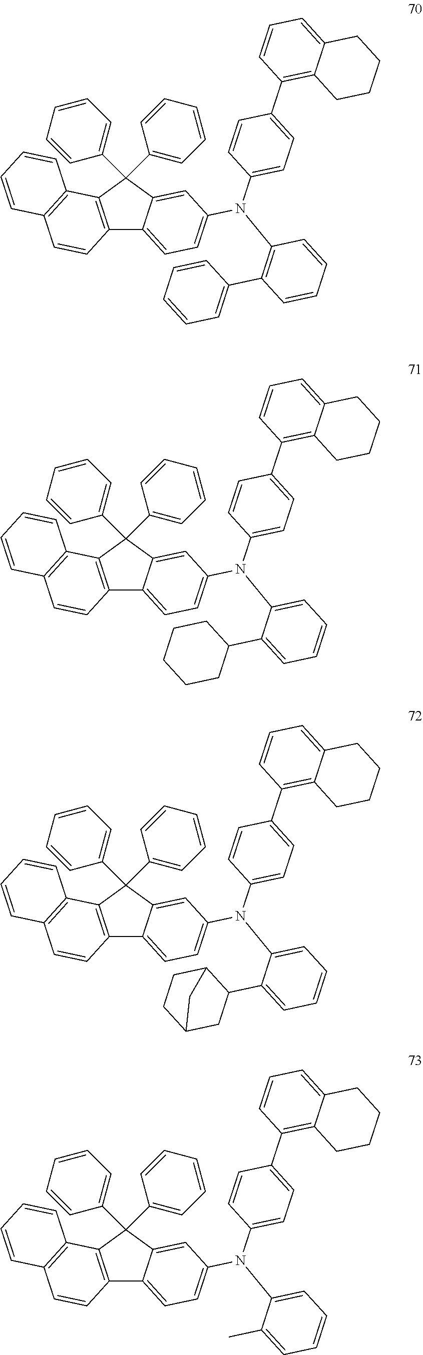 [SMILES: c1ccc(-c2ccccc2N(c2ccc(-c3cccc4c3CCCC4)cc2)c2ccc3c(c2)C(c2ccccc2)(c2ccccc2)c2c-3ccc3ccccc23)cc1 ; c1ccc(C2(c3ccccc3)c3cc(N(c4ccc(-c5cccc6c5CCCC6)cc4)c4ccccc4C4CCCCC4)ccc3-c3ccc4ccccc4c32)cc1 ; c1ccc(C2(c3ccccc3)c3cc(N(c4ccc(-c5cccc6c5CCCC6)cc4)c4ccccc4C4CC5CCC4C5)ccc3-c3ccc4ccccc4c32)cc1 ; Cc1ccccc1N(c1ccc(-c2cccc3c2CCCC3)cc1)c1ccc2c(c1)C(c1ccccc1)(c1ccccc1)c1c-2ccc2ccccc12] 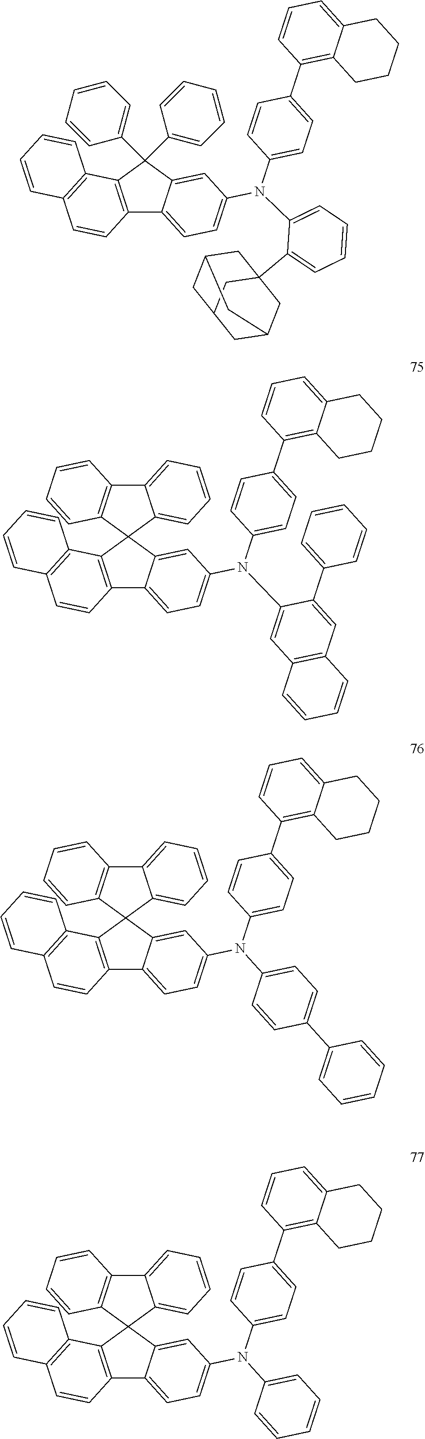 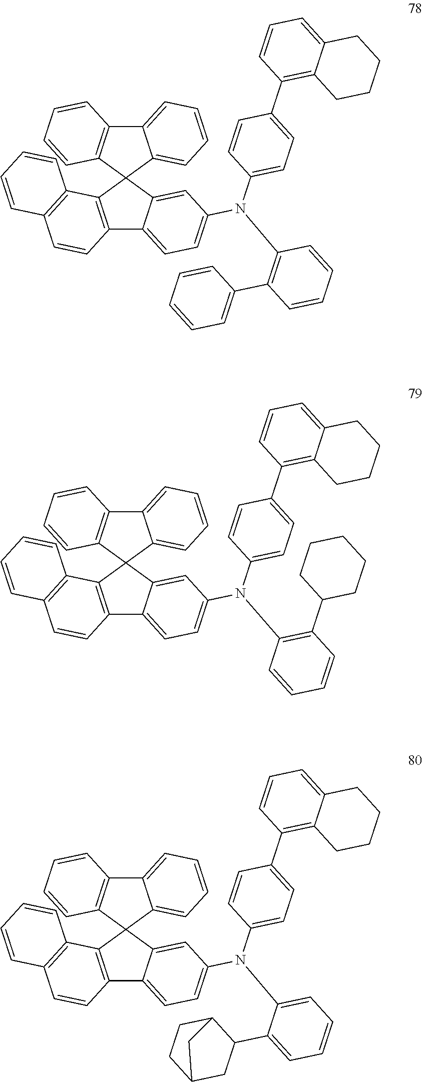 [SMILES: c1ccc(-c2ccccc2N(c2ccc(-c3cccc4c3CCCC4)cc2)c2ccc3c(c2)C2(c4ccccc4-c4ccccc42)c2c-3ccc3ccccc23)cc1 ; c1ccc2c(c1)-c1ccccc1C21c2cc(N(c3ccc(-c4cccc5c4CCCC5)cc3)c3ccccc3C3CCCCC3)ccc2-c2ccc3ccccc3c21 ; c1ccc2c(c1)-c1ccccc1C21c2cc(N(c3ccc(-c4cccc5c4CCCC5)cc3)c3ccccc3C3CC4CCC3C4)ccc2-c2ccc3ccccc3c21] 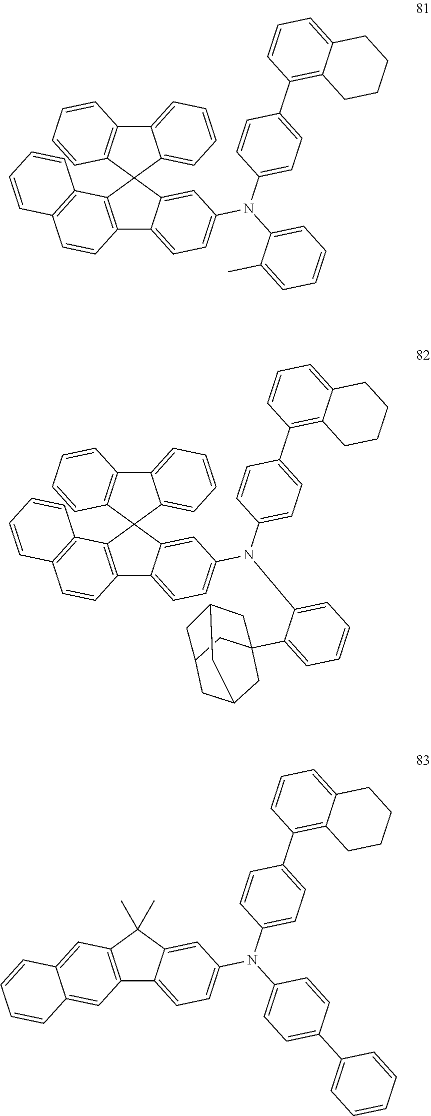 [SMILES: Cc1ccccc1N(c1ccc(-c2cccc3c2CCCC3)cc1)c1ccc2c(c1)C1(c3ccccc3-c3ccccc31)c1c-2ccc2ccccc12 ; c1ccc2c(c1)-c1ccccc1C21c2cc(N(c3ccc(-c4cccc5c4CCCC5)cc3)c3ccccc3C34CC5CC(CC(C5)C3)C4)ccc2-c2ccc3ccccc3c21 ; CC1(C)c2cc(N(c3ccc(-c4ccccc4)cc3)c3ccc(-c4cccc5c4CCCC5)cc3)ccc2-c2cc3ccccc3cc21] 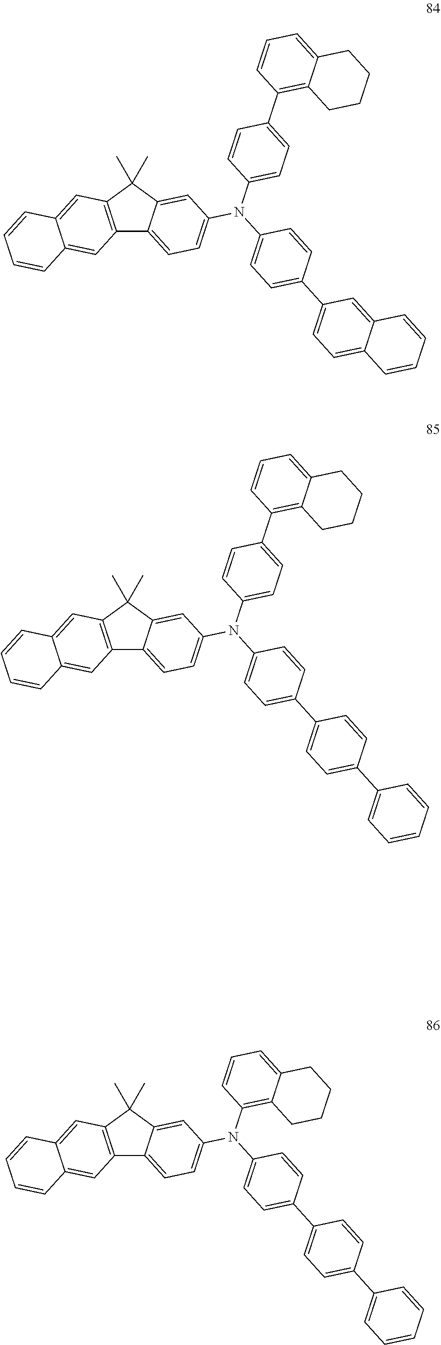 [SMILES: CC1(C)c2cc(N(c3ccc(-c4ccc5ccccc5c4)cc3)c3ccc(-c4cccc5c4CCCC5)cc3)ccc2-c2cc3ccccc3cc21 ; CC1(C)c2cc(N(c3ccc(-c4ccc(-c5ccccc5)cc4)cc3)c3ccc(-c4cccc5c4CCCC5)cc3)ccc2-c2cc3ccccc3cc21 ; CC1(C)c2cc(N(c3ccc(-c4ccc(-c5ccccc5)cc4)cc3)c3cccc4c3CCCC4)ccc2-c2cc3ccccc3cc21] 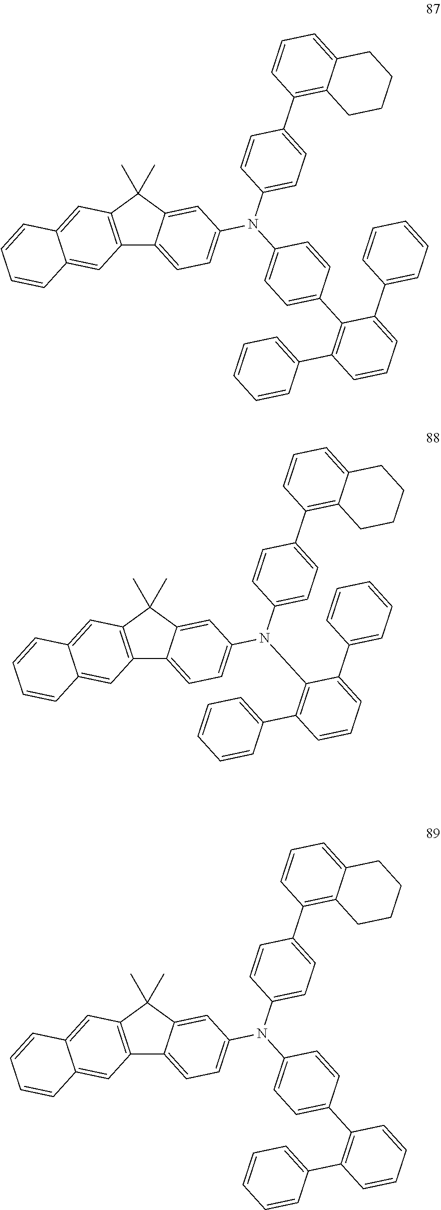 [SMILES: CC1(C)c2cc(N(c3ccc(-c4cccc5c4CCCC5)cc3)c3ccc(-c4c(-c5ccccc5)cccc4-c4ccccc4)cc3)ccc2-c2cc3ccccc3cc21 ; CC1(C)c2cc(N(c3ccc(-c4cccc5c4CCCC5)cc3)c3c(-c4ccccc4)cccc3-c3ccccc3)ccc2-c2cc3ccccc3cc21 ; CC1(C)c2cc(N(c3ccc(-c4ccccc4-c4ccccc4)cc3)c3ccc(-c4cccc5c4CCCC5)cc3)ccc2-c2cc3ccccc3cc21] 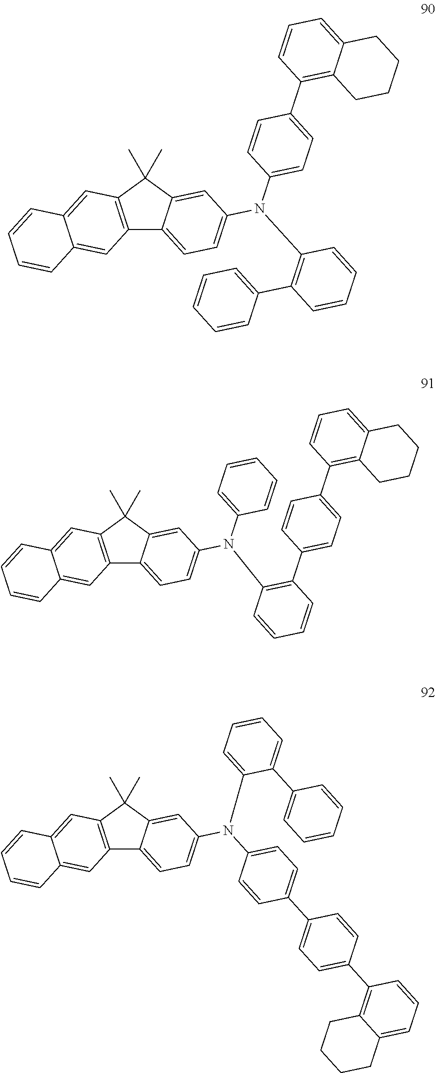 [SMILES: CC1(C)c2cc(N(c3ccc(-c4cccc5c4CCCC5)cc3)c3ccccc3-c3ccccc3)ccc2-c2cc3ccccc3cc21 ; CC1(C)c2cc(N(c3ccccc3)c3ccccc3-c3ccc(-c4cccc5c4CCCC5)cc3)ccc2-c2cc3ccccc3cc21 ; CC1(C)c2cc(N(c3ccc(-c4ccc(-c5cccc6c5CCCC6)cc4)cc3)c3ccccc3-c3ccccc3)ccc2-c2cc3ccccc3cc21] 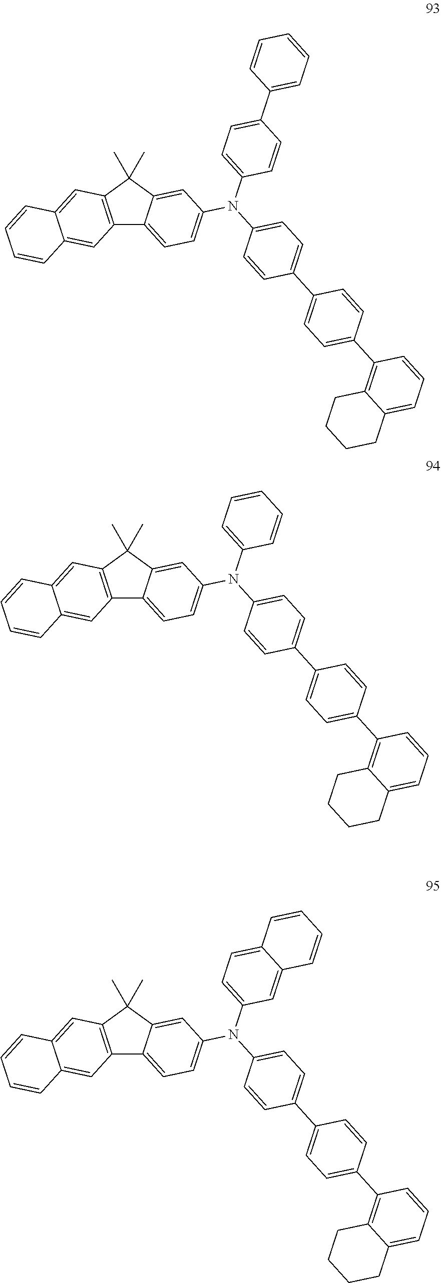 [SMILES: CC1(C)c2cc(N(c3ccc(-c4ccccc4)cc3)c3ccc(-c4ccc(-c5cccc6c5CCCC6)cc4)cc3)ccc2-c2cc3ccccc3cc21 ; CC1(C)c2cc(N(c3ccccc3)c3ccc(-c4ccc(-c5cccc6c5CCCC6)cc4)cc3)ccc2-c2cc3ccccc3cc21 ; CC1(C)c2cc(N(c3ccc(-c4ccc(-c5cccc6c5CCCC6)cc4)cc3)c3ccc4ccccc4c3)ccc2-c2cc3ccccc3cc21] 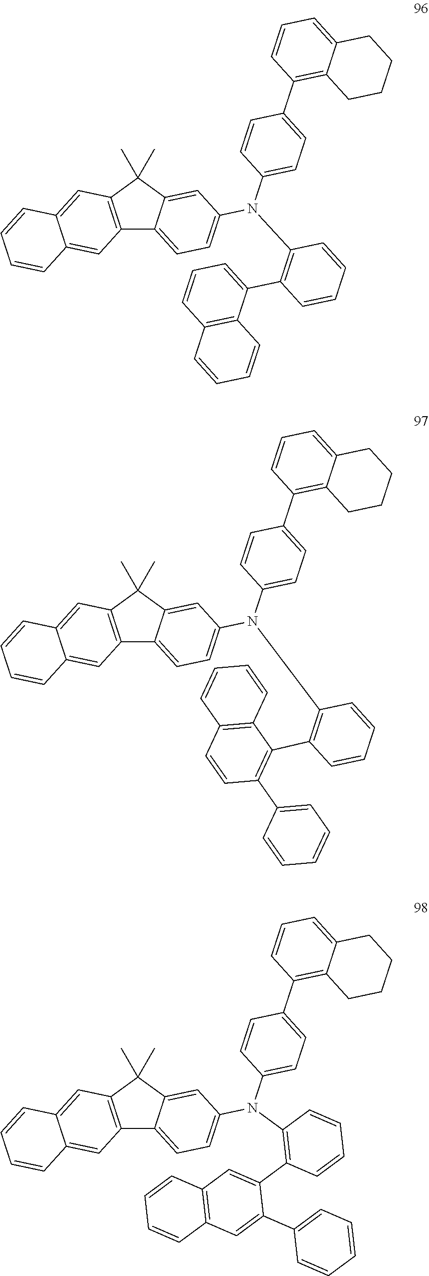 [SMILES: CC1(C)c2cc(N(c3ccc(-c4cccc5c4CCCC5)cc3)c3ccccc3-c3cccc4ccccc34)ccc2-c2cc3ccccc3cc21 ; CC1(C)c2cc(N(c3ccc(-c4cccc5c4CCCC5)cc3)c3ccccc3-c3c(-c4ccccc4)ccc4ccccc34)ccc2-c2cc3ccccc3cc21 ; CC1(C)c2cc(N(c3ccc(-c4cccc5c4CCCC5)cc3)c3ccccc3-c3cc4ccccc4cc3-c3ccccc3)ccc2-c2cc3ccccc3cc21] 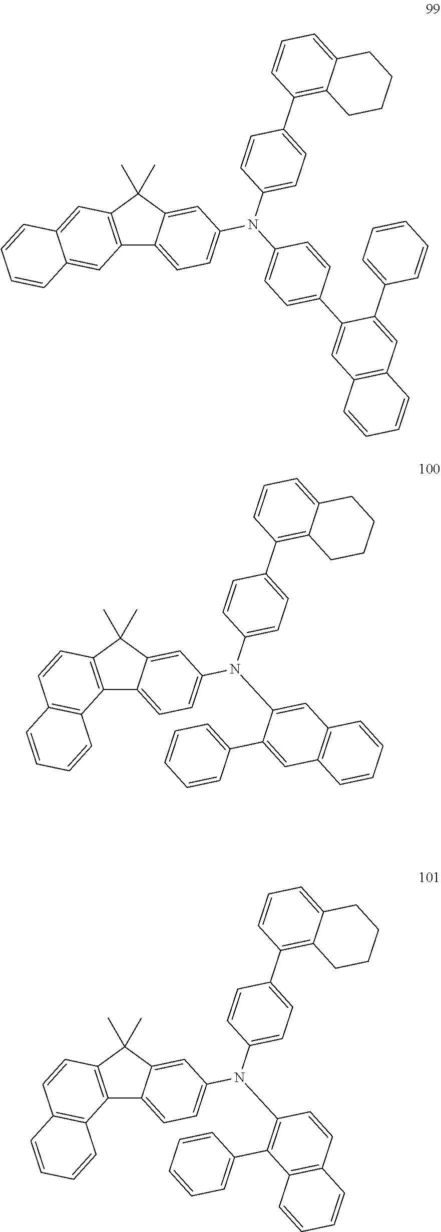 [SMILES: CC1(C)c2cc(N(c3ccc(-c4cc5ccccc5cc4-c4ccccc4)cc3)c3ccc(-c4cccc5c4CCCC5)cc3)ccc2-c2cc3ccccc3cc21 ; CC1(C)c2cc(N(c3ccc(-c4cccc5c4CCCC5)cc3)c3cc4ccccc4cc3-c3ccccc3)ccc2-c2c1ccc1ccccc21 ; CC1(C)c2cc(N(c3ccc(-c4cccc5c4CCCC5)cc3)c3ccc4ccccc4c3-c3ccccc3)ccc2-c2c1ccc1ccccc21] 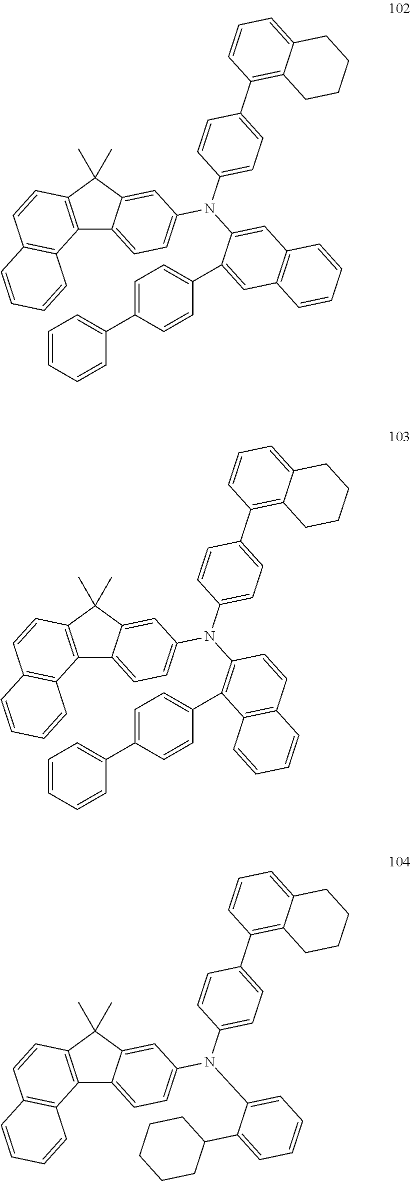 [SMILES: CC1(C)c2cc(N(c3ccc(-c4cccc5c4CCCC5)cc3)c3cc4ccccc4cc3-c3ccc(-c4ccccc4)cc3)ccc2-c2c1ccc1ccccc21 ; CC1(C)c2cc(N(c3ccc(-c4cccc5c4CCCC5)cc3)c3ccc4ccccc4c3-c3ccc(-c4ccccc4)cc3)ccc2-c2c1ccc1ccccc21 ; CC1(C)c2cc(N(c3ccc(-c4cccc5c4CCCC5)cc3)c3ccccc3C3CCCCC3)ccc2-c2c1ccc1ccccc21] 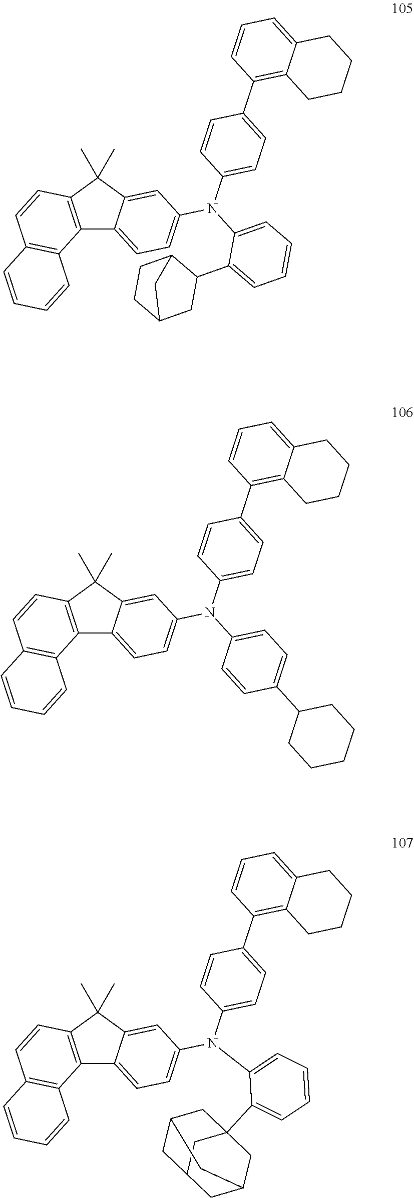 [SMILES: CC1(C)c2cc(N(c3ccc(-c4cccc5c4CCCC5)cc3)c3ccccc3C3CC4CCC3C4)ccc2-c2c1ccc1ccccc21 ; CC1(C)c2cc(N(c3ccc(-c4cccc5c4CCCC5)cc3)c3ccc(C4CCCCC4)cc3)ccc2-c2c1ccc1ccccc21 ; CC1(C)c2cc(N(c3ccc(-c4cccc5c4CCCC5)cc3)c3ccccc3C34CC5CC(CC(C5)C3)C4)ccc2-c2c1ccc1ccccc21] 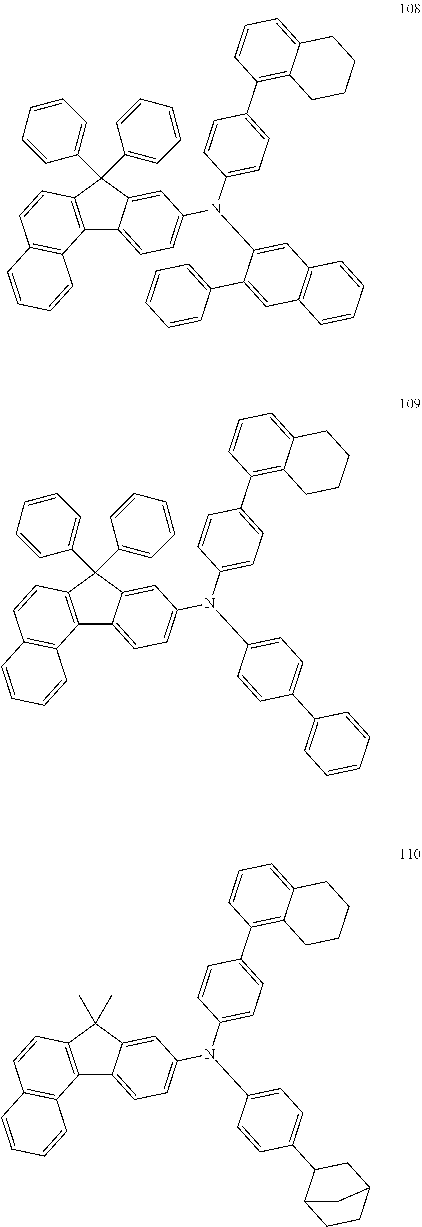 [SMILES: c1ccc(-c2cc3ccccc3cc2N(c2ccc(-c3cccc4c3CCCC4)cc2)c2ccc3c(c2)C(c2ccccc2)(c2ccccc2)c2ccc4ccccc4c2-3)cc1 ; c1ccc(-c2ccc(N(c3ccc(-c4cccc5c4CCCC5)cc3)c3ccc4c(c3)C(c3ccccc3)(c3ccccc3)c3ccc5ccccc5c3-4)cc2)cc1 ; CC1(C)c2cc(N(c3ccc(-c4cccc5c4CCCC5)cc3)c3ccc(C4CC5CCC4C5)cc3)ccc2-c2c1ccc1ccccc21] 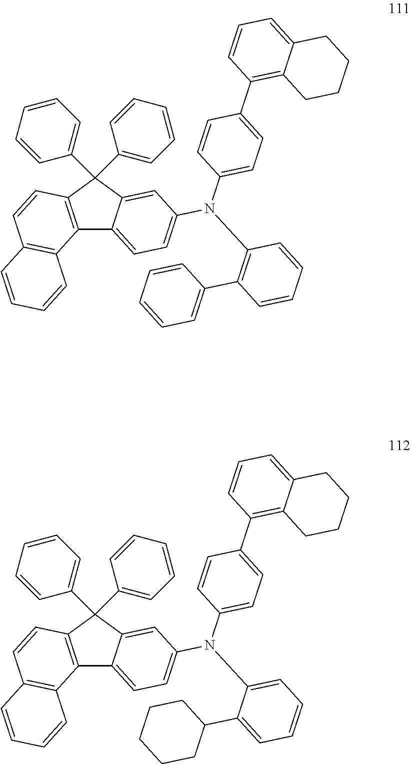 [SMILES: c1ccc(-c2ccccc2N(c2ccc(-c3cccc4c3CCCC4)cc2)c2ccc3c(c2)C(c2ccccc2)(c2ccccc2)c2ccc4ccccc4c2-3)cc1 ; c1ccc(C2(c3ccccc3)c3cc(N(c4ccc(-c5cccc6c5CCCC6)cc4)c4ccccc4C4CCCCC4)ccc3-c3c2ccc2ccccc32)cc1] 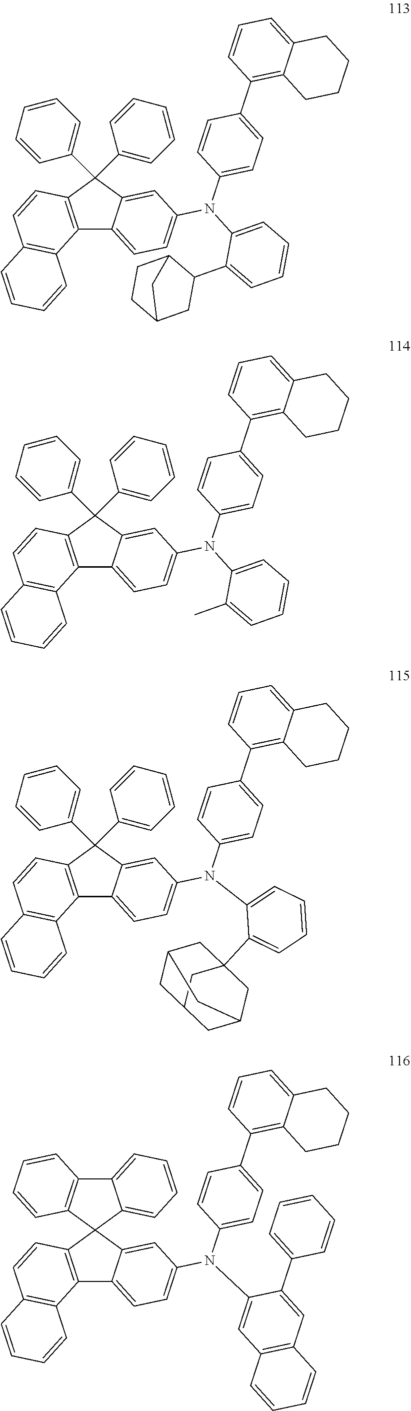 [SMILES: c1ccc(C2(c3ccccc3)c3cc(N(c4ccc(-c5cccc6c5CCCC6)cc4)c4ccccc4C4CC5CCC4C5)ccc3-c3c2ccc2ccccc32)cc1 ; Cc1ccccc1N(c1ccc(-c2cccc3c2CCCC3)cc1)c1ccc2c(c1)C(c1ccccc1)(c1ccccc1)c1ccc3ccccc3c1-2 ; c1ccc(C2(c3ccccc3)c3cc(N(c4ccc(-c5cccc6c5CCCC6)cc4)c4ccccc4C45CC6CC(CC(C6)C4)C5)ccc3-c3c2ccc2ccccc32)cc1 ; c1ccc(-c2cc3ccccc3cc2N(c2ccc(-c3cccc4c3CCCC4)cc2)c2ccc3c(c2)C2(c4ccccc4-c4ccccc42)c2ccc4ccccc4c2-3)cc1] 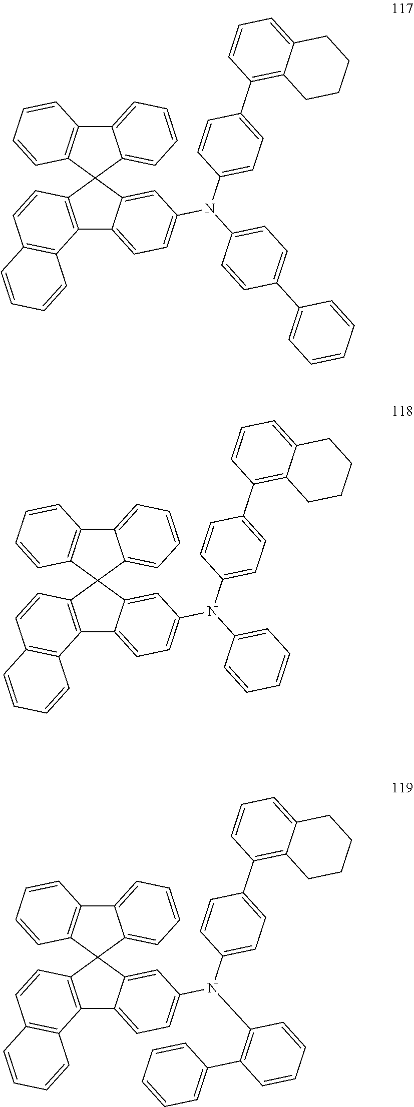 [SMILES: c1ccc(-c2ccc(N(c3ccc(-c4cccc5c4CCCC5)cc3)c3ccc4c(c3)C3(c5ccccc5-c5ccccc53)c3ccc5ccccc5c3-4)cc2)cc1 ; c1ccc(N(c2ccc(-c3cccc4c3CCCC4)cc2)c2ccc3c(c2)C2(c4ccccc4-c4ccccc42)c2ccc4ccccc4c2-3)cc1 ; c1ccc(-c2ccccc2N(c2ccc(-c3cccc4c3CCCC4)cc2)c2ccc3c(c2)C2(c4ccccc4-c4ccccc42)c2ccc4ccccc4c2-3)cc1] 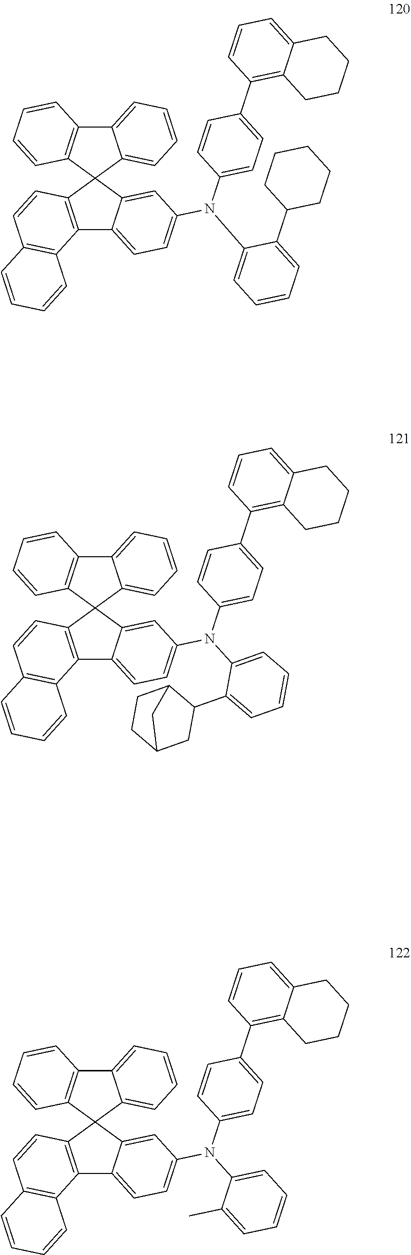 [SMILES: c1ccc2c(c1)-c1ccccc1C21c2cc(N(c3ccc(-c4cccc5c4CCCC5)cc3)c3ccccc3C3CCCCC3)ccc2-c2c1ccc1ccccc21 ; c1ccc2c(c1)-c1ccccc1C21c2cc(N(c3ccc(-c4cccc5c4CCCC5)cc3)c3ccccc3C3CC4CCC3C4)ccc2-c2c1ccc1ccccc21 ; Cc1ccccc1N(c1ccc(-c2cccc3c2CCCC3)cc1)c1ccc2c(c1)C1(c3ccccc3-c3ccccc31)c1ccc3ccccc3c1-2] 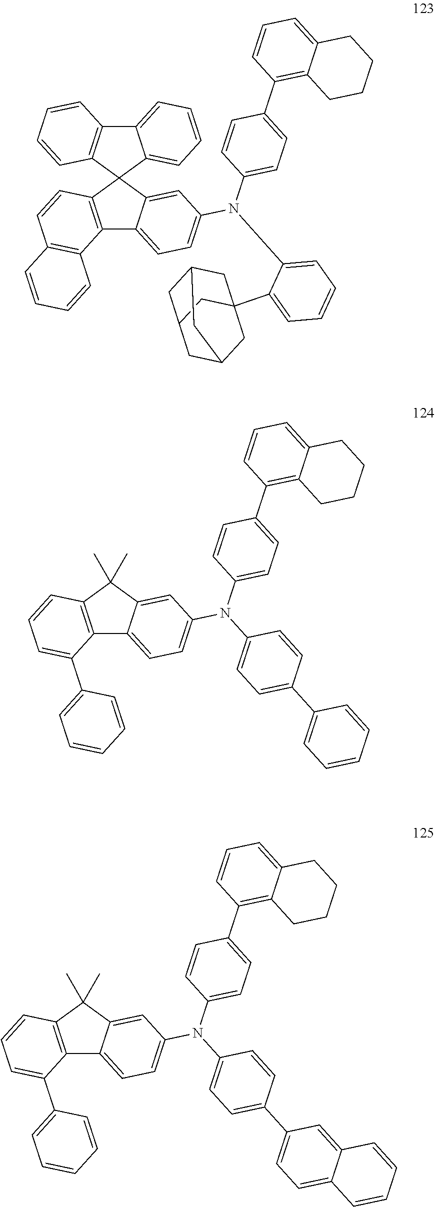 [SMILES: c1ccc2c(c1)-c1ccccc1C21c2cc(N(c3ccc(-c4cccc5c4CCCC5)cc3)c3ccccc3C34CC5CC(CC(C5)C3)C4)ccc2-c2c1ccc1ccccc21 ; CC1(C)c2cc(N(c3ccc(-c4ccccc4)cc3)c3ccc(-c4cccc5c4CCCC5)cc3)ccc2-c2c(-c3ccccc3)cccc21 ; CC1(C)c2cc(N(c3ccc(-c4ccc5ccccc5c4)cc3)c3ccc(-c4cccc5c4CCCC5)cc3)ccc2-c2c(-c3ccccc3)cccc21] 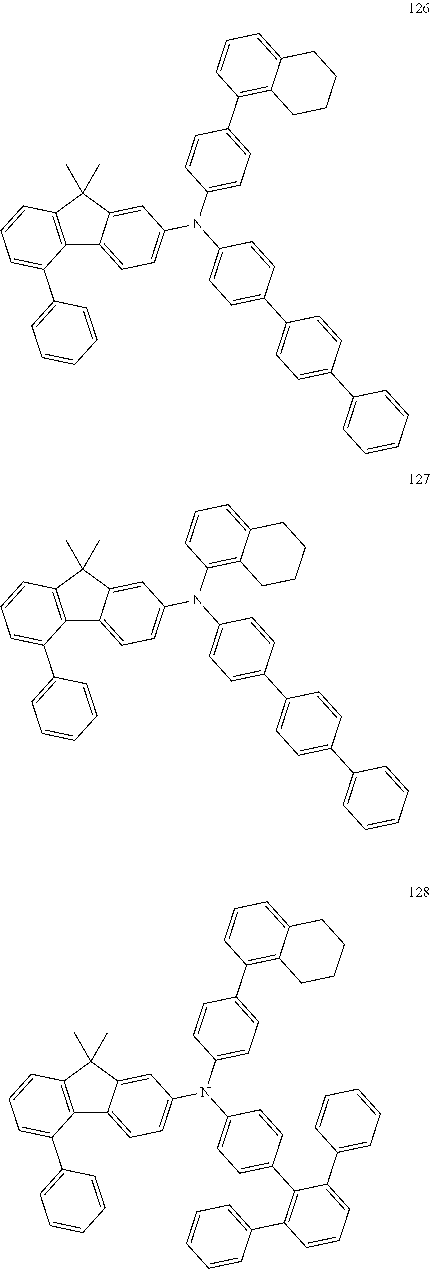 [SMILES: CC1(C)c2cc(N(c3ccc(-c4ccc(-c5ccccc5)cc4)cc3)c3ccc(-c4cccc5c4CCCC5)cc3)ccc2-c2c(-c3ccccc3)cccc21 ; CC1(C)c2cc(N(c3ccc(-c4ccc(-c5ccccc5)cc4)cc3)c3cccc4c3CCCC4)ccc2-c2c(-c3ccccc3)cccc21 ; CC1(C)c2cc(N(c3ccc(-c4cccc5c4CCCC5)cc3)c3ccc(-c4c(-c5ccccc5)cccc4-c4ccccc4)cc3)ccc2-c2c(-c3ccccc3)cccc21] 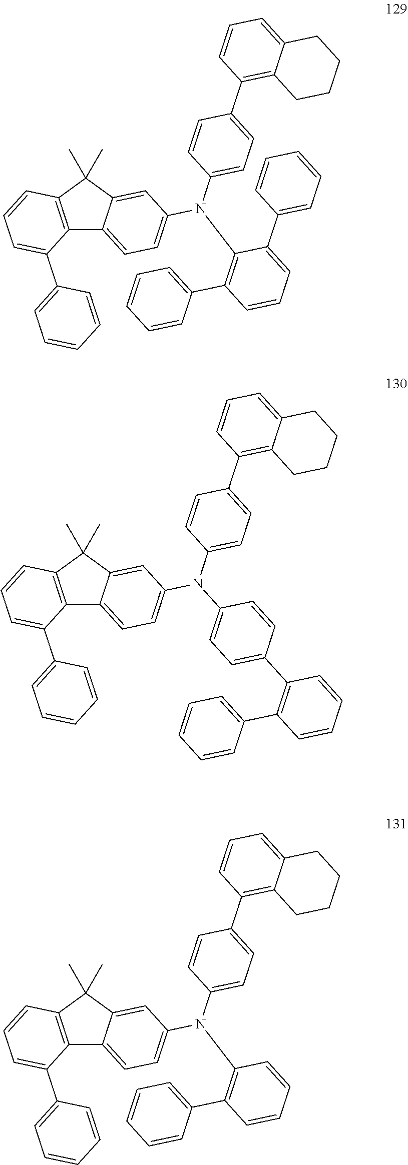 [SMILES: CC1(C)c2cc(N(c3ccc(-c4cccc5c4CCCC5)cc3)c3c(-c4ccccc4)cccc3-c3ccccc3)ccc2-c2c(-c3ccccc3)cccc21 ; CC1(C)c2cc(N(c3ccc(-c4ccccc4-c4ccccc4)cc3)c3ccc(-c4cccc5c4CCCC5)cc3)ccc2-c2c(-c3ccccc3)cccc21 ; CC1(C)c2cc(N(c3ccc(-c4cccc5c4CCCC5)cc3)c3ccccc3-c3ccccc3)ccc2-c2c(-c3ccccc3)cccc21] 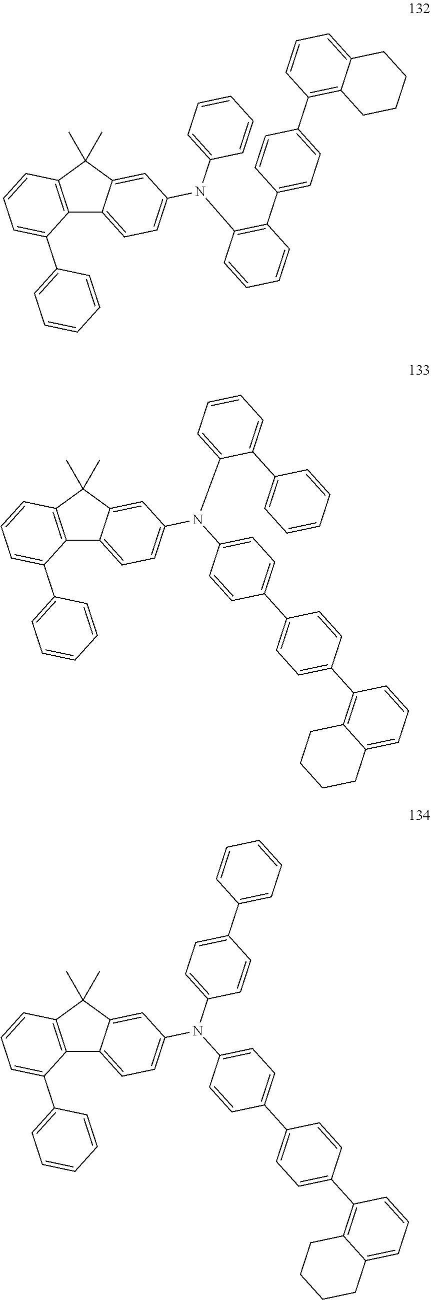 [SMILES: CC1(C)c2cc(N(c3ccccc3)c3ccccc3-c3ccc(-c4cccc5c4CCCC5)cc3)ccc2-c2c(-c3ccccc3)cccc21 ; CC1(C)c2cc(N(c3ccc(-c4ccc(-c5cccc6c5CCCC6)cc4)cc3)c3ccccc3-c3ccccc3)ccc2-c2c(-c3ccccc3)cccc21 ; CC1(C)c2cc(N(c3ccc(-c4ccccc4)cc3)c3ccc(-c4ccc(-c5cccc6c5CCCC6)cc4)cc3)ccc2-c2c(-c3ccccc3)cccc21] 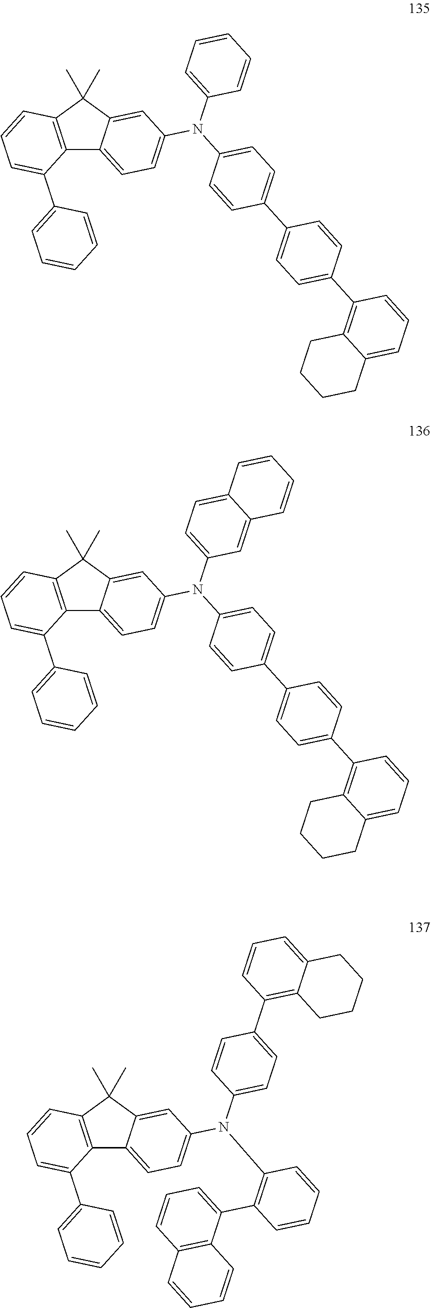 [SMILES: CC1(C)c2cc(N(c3ccccc3)c3ccc(-c4ccc(-c5cccc6c5CCCC6)cc4)cc3)ccc2-c2c(-c3ccccc3)cccc21 ; CC1(C)c2cc(N(c3ccc(-c4ccc(-c5cccc6c5CCCC6)cc4)cc3)c3ccc4ccccc4c3)ccc2-c2c(-c3ccccc3)cccc21 ; CC1(C)c2cc(N(c3ccc(-c4cccc5c4CCCC5)cc3)c3ccccc3-c3cccc4ccccc34)ccc2-c2c(-c3ccccc3)cccc21] 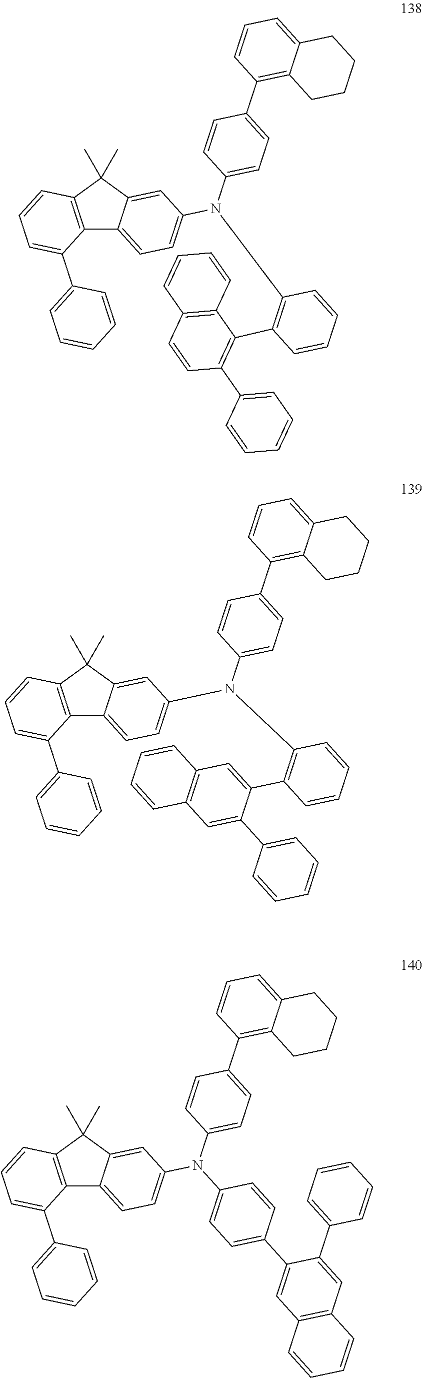 [SMILES: CC1(C)c2cc(N(c3ccc(-c4cccc5c4CCCC5)cc3)c3ccccc3-c3c(-c4ccccc4)ccc4ccccc34)ccc2-c2c(-c3ccccc3)cccc21 ; CC1(C)c2cc(N(c3ccc(-c4cccc5c4CCCC5)cc3)c3ccccc3-c3cc4ccccc4cc3-c3ccccc3)ccc2-c2c(-c3ccccc3)cccc21 ; CC1(C)c2cc(N(c3ccc(-c4cc5ccccc5cc4-c4ccccc4)cc3)c3ccc(-c4cccc5c4CCCC5)cc3)ccc2-c2c(-c3ccccc3)cccc21] 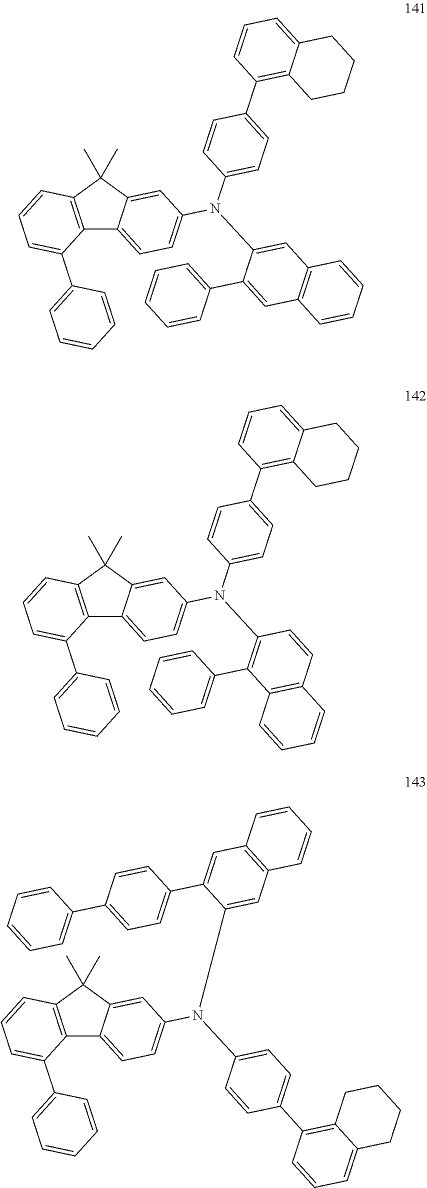 [SMILES: CC1(C)c2cc(N(c3ccc(-c4cccc5c4CCCC5)cc3)c3cc4ccccc4cc3-c3ccccc3)ccc2-c2c(-c3ccccc3)cccc21 ; CC1(C)c2cc(N(c3ccc(-c4cccc5c4CCCC5)cc3)c3ccc4ccccc4c3-c3ccccc3)ccc2-c2c(-c3ccccc3)cccc21 ; CC1(C)c2cc(N(c3ccc(-c4cccc5c4CCCC5)cc3)c3cc4ccccc4cc3-c3ccc(-c4ccccc4)cc3)ccc2-c2c(-c3ccccc3)cccc21] 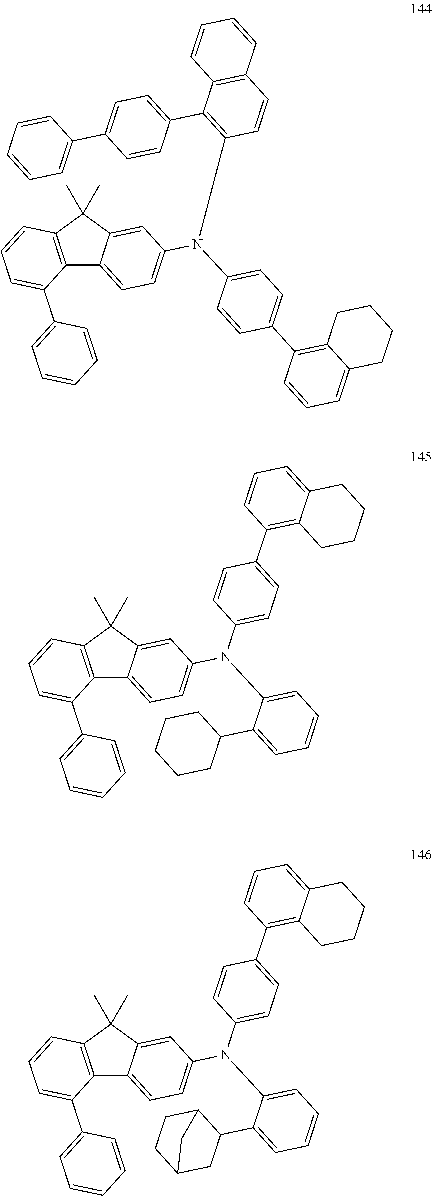 [SMILES: CC1(C)c2cc(N(c3ccc(-c4cccc5c4CCCC5)cc3)c3ccc4ccccc4c3-c3ccc(-c4ccccc4)cc3)ccc2-c2c(-c3ccccc3)cccc21 ; CC1(C)c2cc(N(c3ccc(-c4cccc5c4CCCC5)cc3)c3ccccc3C3CCCCC3)ccc2-c2c(-c3ccccc3)cccc21 ; CC1(C)c2cc(N(c3ccc(-c4cccc5c4CCCC5)cc3)c3ccccc3C3CC4CCC3C4)ccc2-c2c(-c3ccccc3)cccc21] 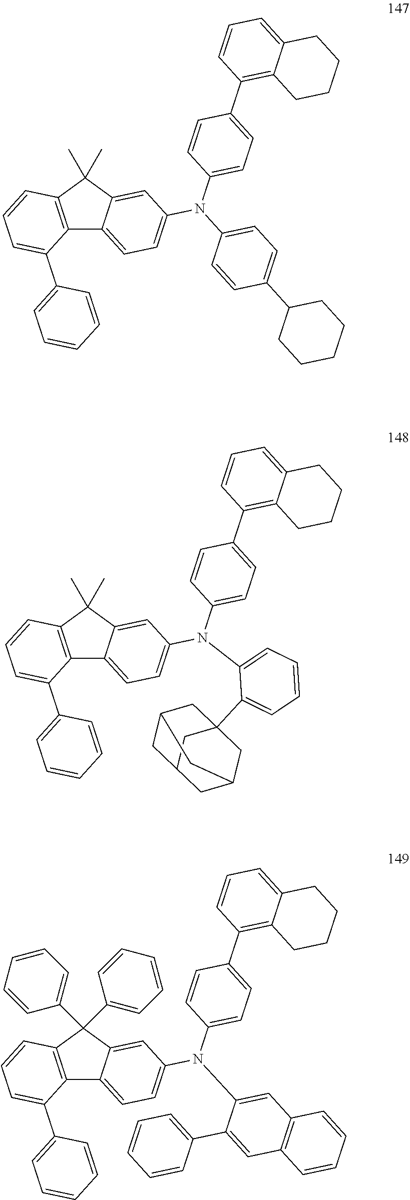 [SMILES: CC1(C)c2cc(N(c3ccc(-c4cccc5c4CCCC5)cc3)c3ccc(C4CCCCC4)cc3)ccc2-c2c(-c3ccccc3)cccc21 ; CC1(C)c2cc(N(c3ccc(-c4cccc5c4CCCC5)cc3)c3ccccc3C34CC5CC(CC(C5)C3)C4)ccc2-c2c(-c3ccccc3)cccc21 ; c1ccc(-c2cc3ccccc3cc2N(c2ccc(-c3cccc4c3CCCC4)cc2)c2ccc3c(c2)C(c2ccccc2)(c2ccccc2)c2cccc(-c4ccccc4)c2-3)cc1] 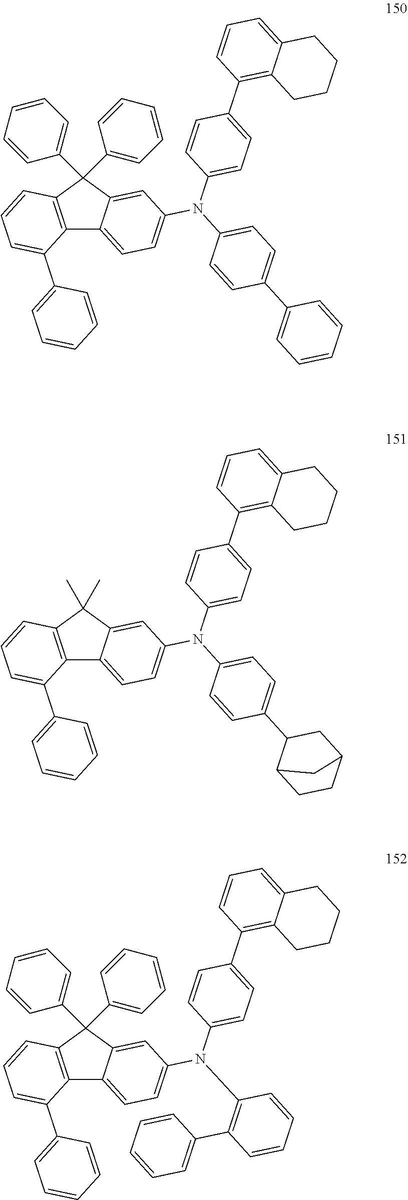 [SMILES: c1ccc(-c2ccc(N(c3ccc(-c4cccc5c4CCCC5)cc3)c3ccc4c(c3)C(c3ccccc3)(c3ccccc3)c3cccc(-c5ccccc5)c3-4)cc2)cc1 ; CC1(C)c2cc(N(c3ccc(-c4cccc5c4CCCC5)cc3)c3ccc(C4CC5CCC4C5)cc3)ccc2-c2c(-c3ccccc3)cccc21 ; c1ccc(-c2ccccc2N(c2ccc(-c3cccc4c3CCCC4)cc2)c2ccc3c(c2)C(c2ccccc2)(c2ccccc2)c2cccc(-c4ccccc4)c2-3)cc1] 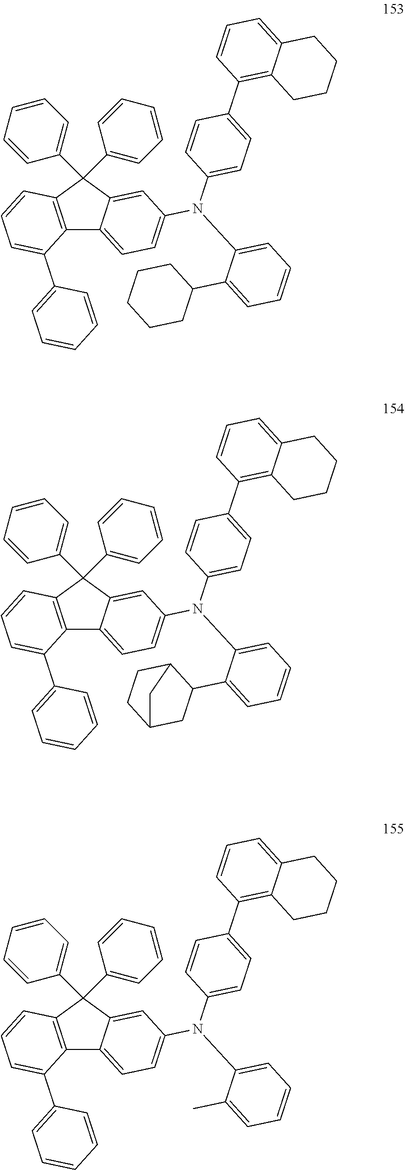 [SMILES: c1ccc(-c2cccc3c2-c2ccc(N(c4ccc(-c5cccc6c5CCCC6)cc4)c4ccccc4C4CCCCC4)cc2C3(c2ccccc2)c2ccccc2)cc1 ; c1ccc(-c2cccc3c2-c2ccc(N(c4ccc(-c5cccc6c5CCCC6)cc4)c4ccccc4C4CC5CCC4C5)cc2C3(c2ccccc2)c2ccccc2)cc1 ; Cc1ccccc1N(c1ccc(-c2cccc3c2CCCC3)cc1)c1ccc2c(c1)C(c1ccccc1)(c1ccccc1)c1cccc(-c3ccccc3)c1-2] 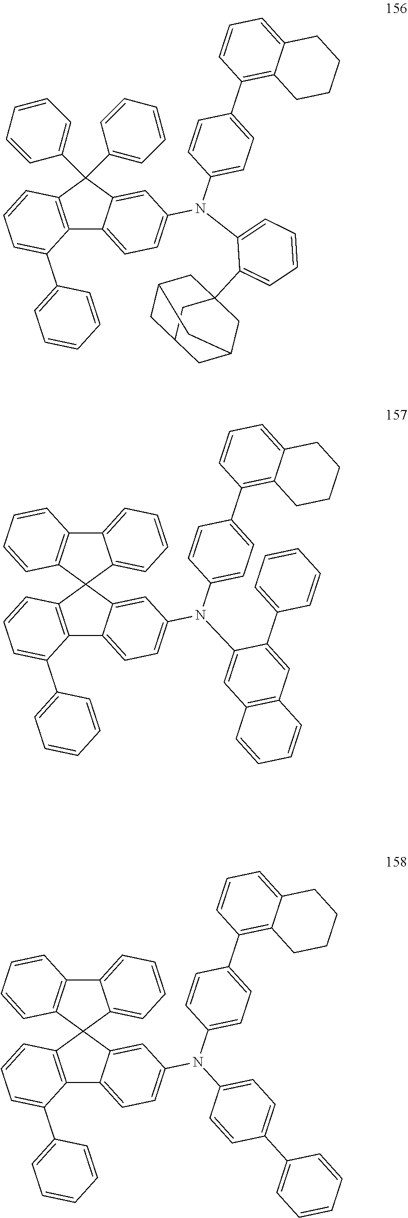 [SMILES: c1ccc(-c2cccc3c2-c2ccc(N(c4ccc(-c5cccc6c5CCCC6)cc4)c4ccccc4C45CC6CC(CC(C6)C4)C5)cc2C3(c2ccccc2)c2ccccc2)cc1 ; c1ccc(-c2cc3ccccc3cc2N(c2ccc(-c3cccc4c3CCCC4)cc2)c2ccc3c(c2)C2(c4ccccc4-c4ccccc42)c2cccc(-c4ccccc4)c2-3)cc1 ; c1ccc(-c2ccc(N(c3ccc(-c4cccc5c4CCCC5)cc3)c3ccc4c(c3)C3(c5ccccc5-c5ccccc53)c3cccc(-c5ccccc5)c3-4)cc2)cc1] 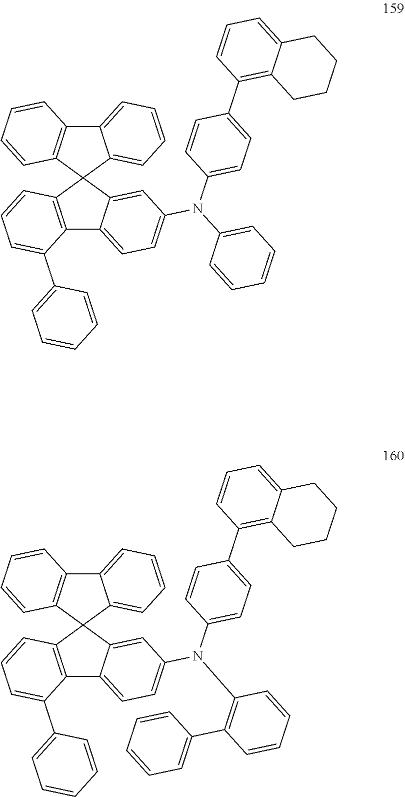 [SMILES: c1ccc(-c2cccc3c2-c2ccc(N(c4ccccc4)c4ccc(-c5cccc6c5CCCC6)cc4)cc2C32c3ccccc3-c3ccccc32)cc1 ; c1ccc(-c2ccccc2N(c2ccc(-c3cccc4c3CCCC4)cc2)c2ccc3c(c2)C2(c4ccccc4-c4ccccc42)c2cccc(-c4ccccc4)c2-3)cc1] 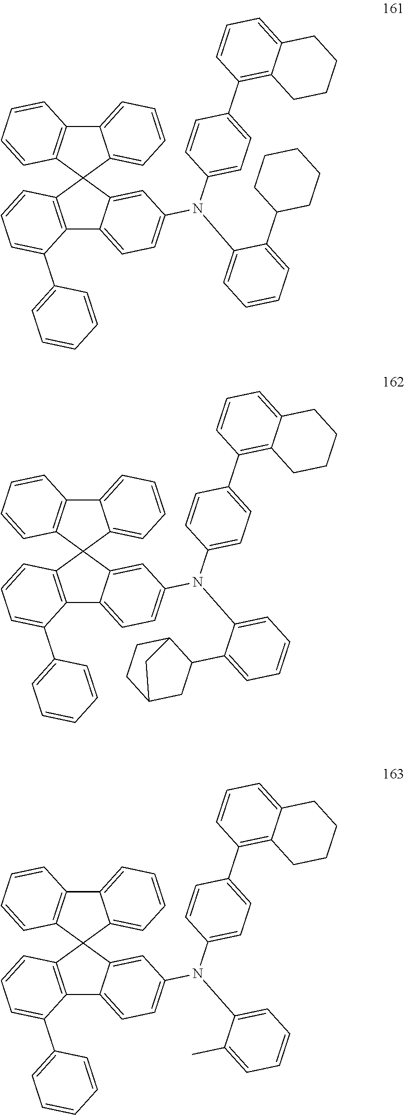 [SMILES: c1ccc(-c2cccc3c2-c2ccc(N(c4ccc(-c5cccc6c5CCCC6)cc4)c4ccccc4C4CCCCC4)cc2C32c3ccccc3-c3ccccc32)cc1 ; c1ccc(-c2cccc3c2-c2ccc(N(c4ccc(-c5cccc6c5CCCC6)cc4)c4ccccc4C4CC5CCC4C5)cc2C32c3ccccc3-c3ccccc32)cc1 ; Cc1ccccc1N(c1ccc(-c2cccc3c2CCCC3)cc1)c1ccc2c(c1)C1(c3ccccc3-c3ccccc31)c1cccc(-c3ccccc3)c1-2] 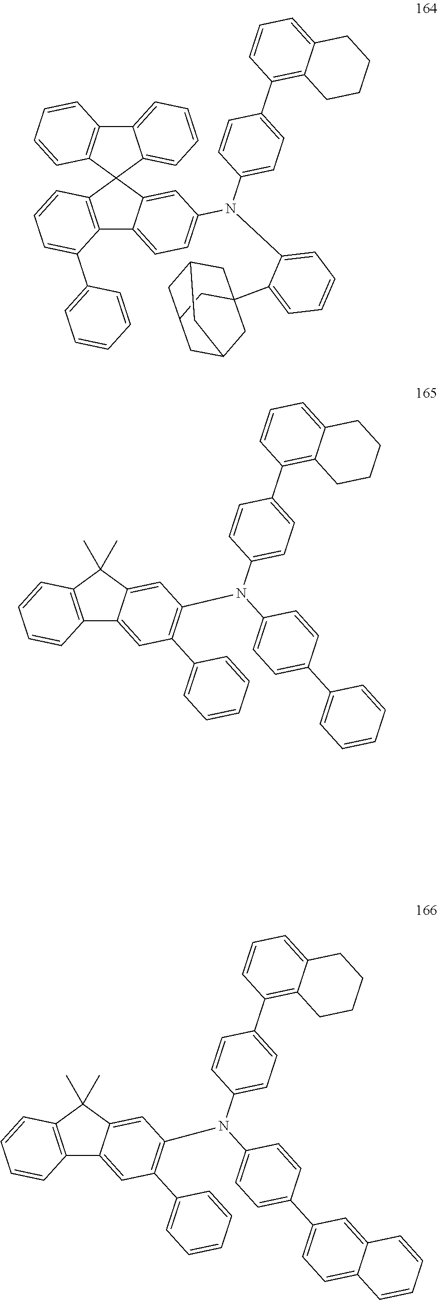 [SMILES: c1ccc(-c2cccc3c2-c2ccc(N(c4ccc(-c5cccc6c5CCCC6)cc4)c4ccccc4C45CC6CC(CC(C6)C4)C5)cc2C32c3ccccc3-c3ccccc32)cc1 ; CC1(C)c2ccccc2-c2cc(-c3ccccc3)c(N(c3ccc(-c4ccccc4)cc3)c3ccc(-c4cccc5c4CCCC5)cc3)cc21 ; CC1(C)c2ccccc2-c2cc(-c3ccccc3)c(N(c3ccc(-c4ccc5ccccc5c4)cc3)c3ccc(-c4cccc5c4CCCC5)cc3)cc21] 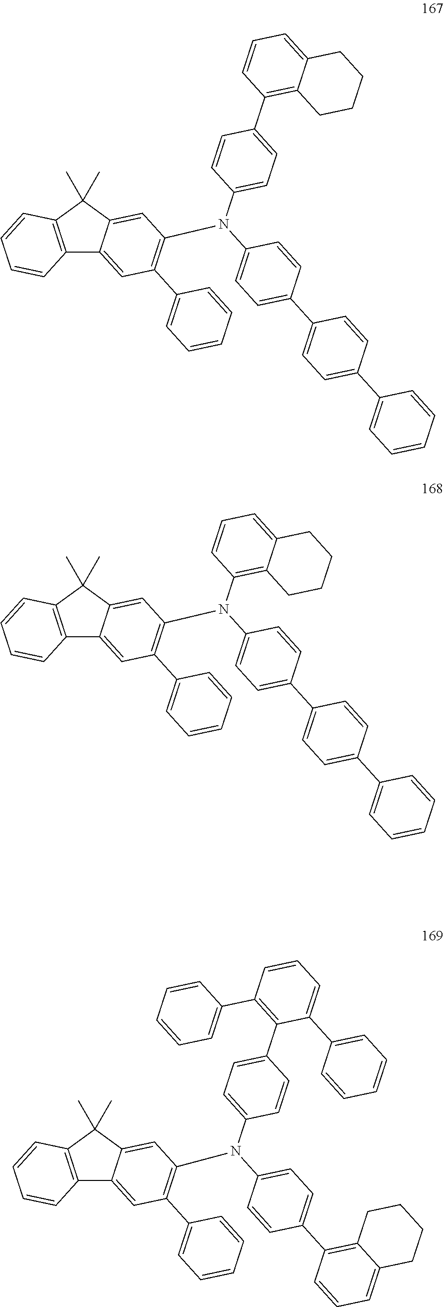 [SMILES: CC1(C)c2ccccc2-c2cc(-c3ccccc3)c(N(c3ccc(-c4ccc(-c5ccccc5)cc4)cc3)c3ccc(-c4cccc5c4CCCC5)cc3)cc21 ; CC1(C)c2ccccc2-c2cc(-c3ccccc3)c(N(c3ccc(-c4ccc(-c5ccccc5)cc4)cc3)c3cccc4c3CCCC4)cc21 ; CC1(C)c2ccccc2-c2cc(-c3ccccc3)c(N(c3ccc(-c4cccc5c4CCCC5)cc3)c3ccc(-c4c(-c5ccccc5)cccc4-c4ccccc4)cc3)cc21] 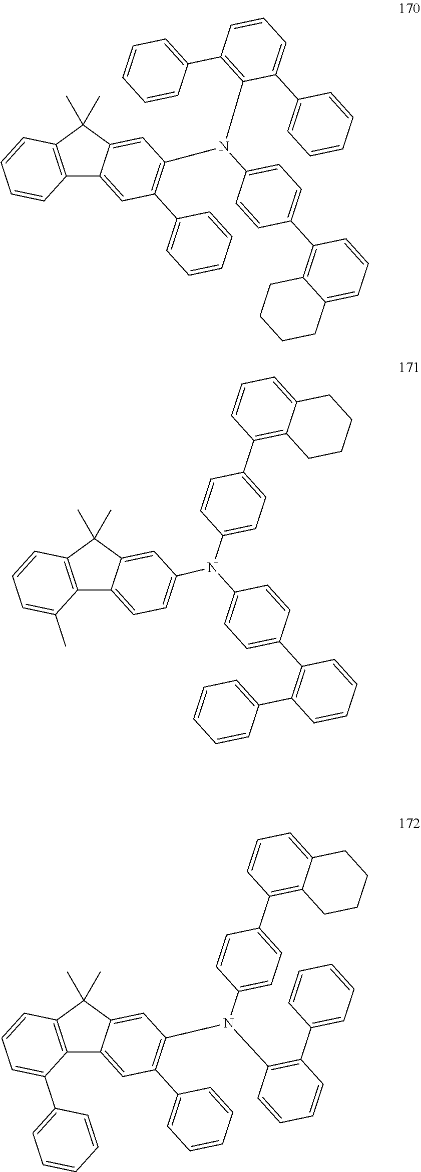 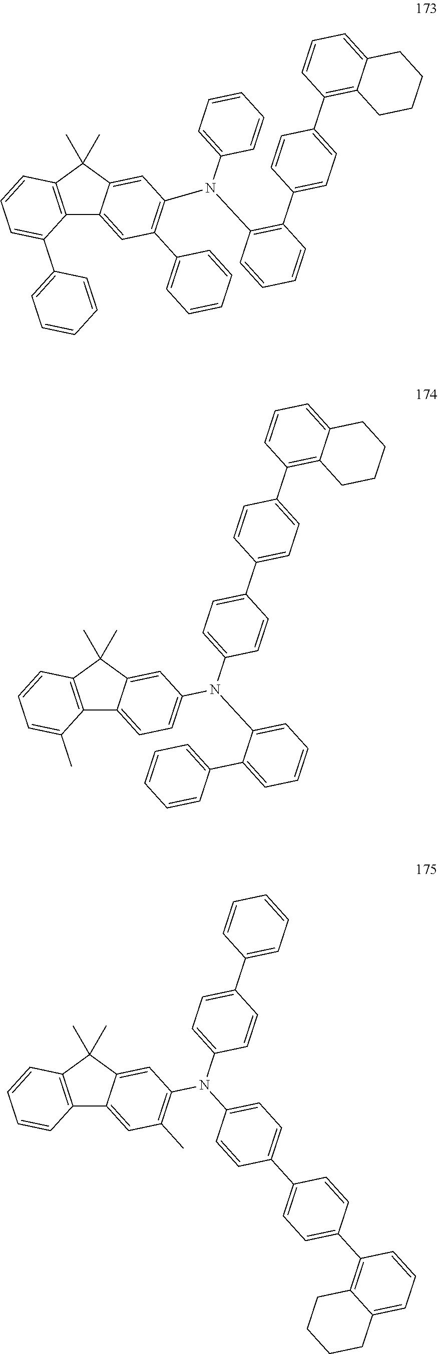 [SMILES: CC1(C)c2cc(N(c3ccccc3)c3ccccc3-c3ccc(-c4cccc5c4CCCC5)cc3)c(-c3ccccc3)cc2-c2c(-c3ccccc3)cccc21 ; Cc1cccc2c1-c1ccc(N(c3ccc(-c4ccc(-c5cccc6c5CCCC6)cc4)cc3)c3ccccc3-c3ccccc3)cc1C2(C)C ; Cc1cc2c(cc1N(c1ccc(-c3ccccc3)cc1)c1ccc(-c3ccc(-c4cccc5c4CCCC5)cc3)cc1)C(C)(C)c1ccccc1-2] 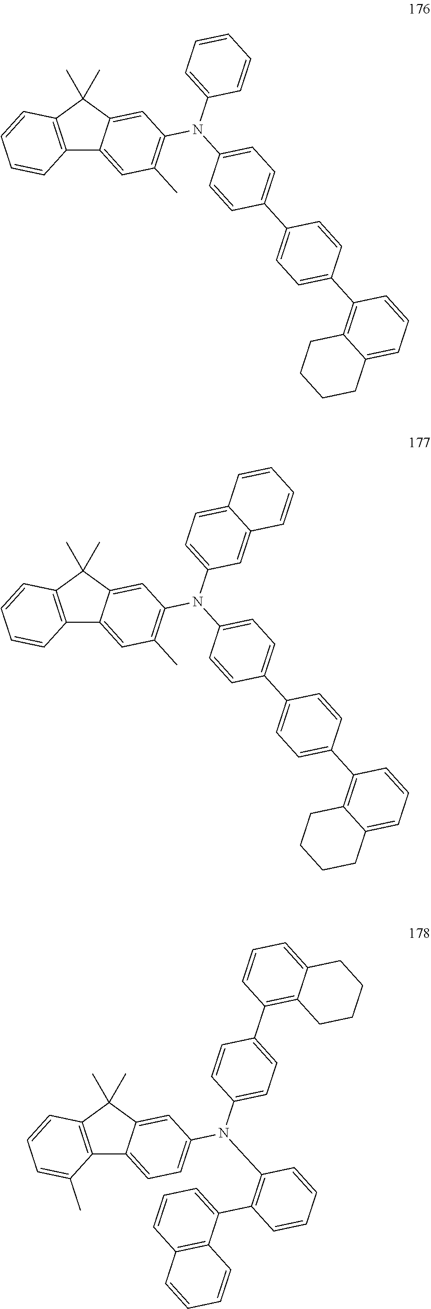 [SMILES: Cc1cc2c(cc1N(c1ccccc1)c1ccc(-c3ccc(-c4cccc5c4CCCC5)cc3)cc1)C(C)(C)c1ccccc1-2 ; Cc1cc2c(cc1N(c1ccc(-c3ccc(-c4cccc5c4CCCC5)cc3)cc1)c1ccc3ccccc3c1)C(C)(C)c1ccccc1-2 ; Cc1cccc2c1-c1ccc(N(c3ccc(-c4cccc5c4CCCC5)cc3)c3ccccc3-c3cccc4ccccc34)cc1C2(C)C] 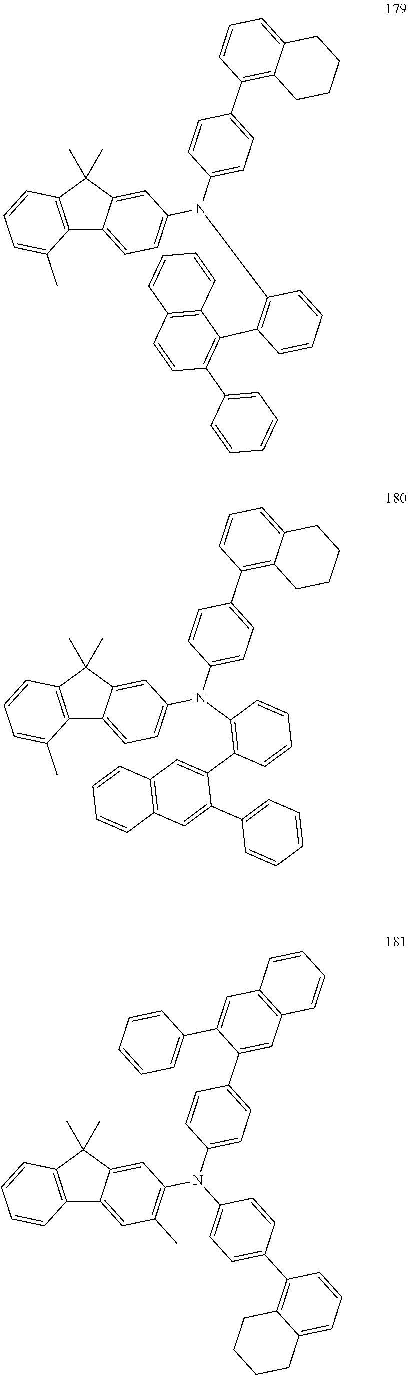 [SMILES: Cc1cccc2c1-c1ccc(N(c3ccc(-c4cccc5c4CCCC5)cc3)c3ccccc3-c3c(-c4ccccc4)ccc4ccccc34)cc1C2(C)C ; Cc1cccc2c1-c1ccc(N(c3ccc(-c4cccc5c4CCCC5)cc3)c3ccccc3-c3cc4ccccc4cc3-c3ccccc3)cc1C2(C)C ; Cc1cc2c(cc1N(c1ccc(-c3cc4ccccc4cc3-c3ccccc3)cc1)c1ccc(-c3cccc4c3CCCC4)cc1)C(C)(C)c1ccccc1-2] 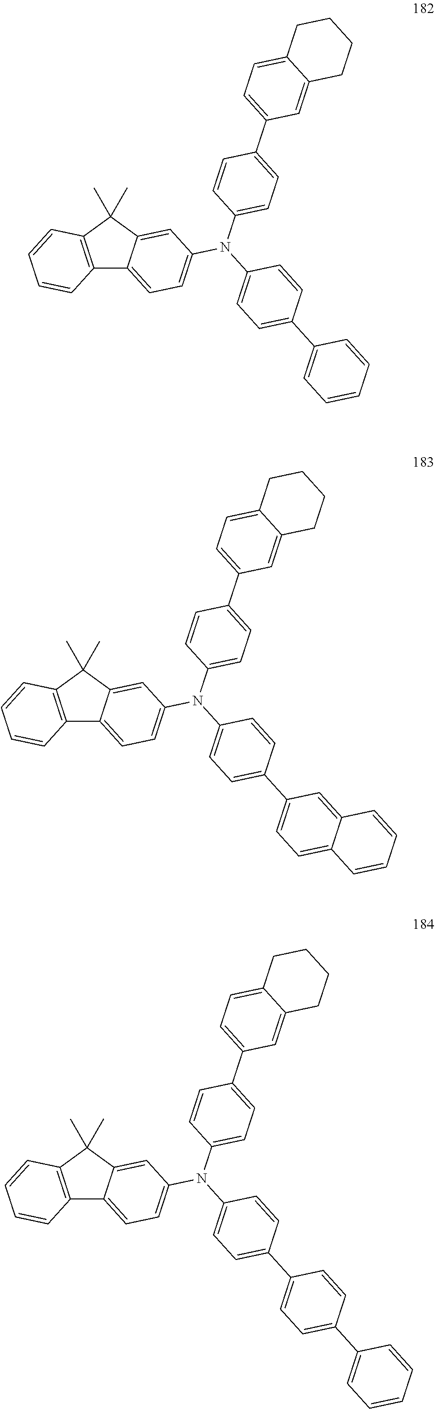 [SMILES: CC1(C)c2ccccc2-c2ccc(N(c3ccc(-c4ccccc4)cc3)c3ccc(-c4ccc5c(c4)CCCC5)cc3)cc21 ; CC1(C)c2ccccc2-c2ccc(N(c3ccc(-c4ccc5c(c4)CCCC5)cc3)c3ccc(-c4ccc5ccccc5c4)cc3)cc21 ; CC1(C)c2ccccc2-c2ccc(N(c3ccc(-c4ccc(-c5ccccc5)cc4)cc3)c3ccc(-c4ccc5c(c4)CCCC5)cc3)cc21] 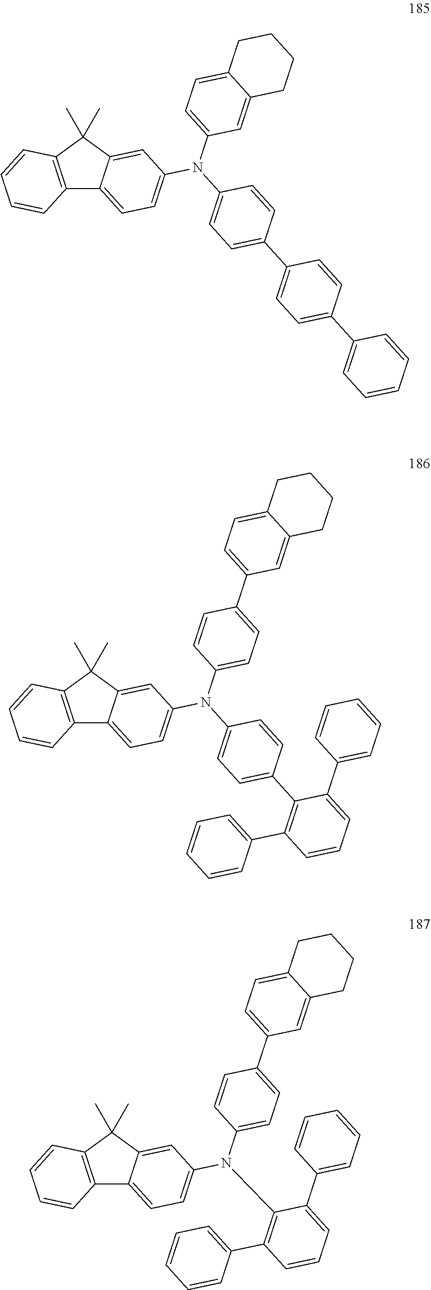 [SMILES: CC1(C)c2ccccc2-c2ccc(N(c3ccc(-c4ccc(-c5ccccc5)cc4)cc3)c3ccc4c(c3)CCCC4)cc21 ; CC1(C)c2ccccc2-c2ccc(N(c3ccc(-c4ccc5c(c4)CCCC5)cc3)c3ccc(-c4c(-c5ccccc5)cccc4-c4ccccc4)cc3)cc21 ; CC1(C)c2ccccc2-c2ccc(N(c3ccc(-c4ccc5c(c4)CCCC5)cc3)c3c(-c4ccccc4)cccc3-c3ccccc3)cc21] 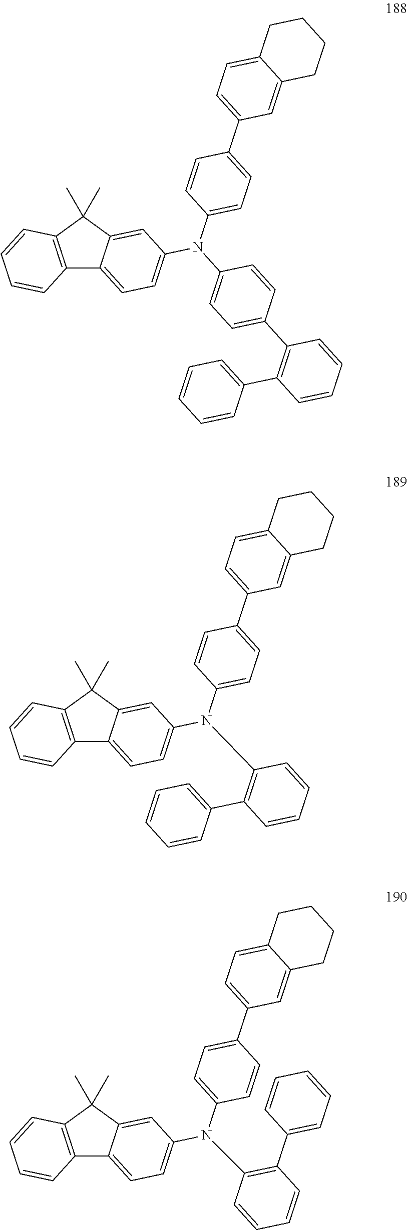 [SMILES: CC1(C)c2ccccc2-c2ccc(N(c3ccc(-c4ccc5c(c4)CCCC5)cc3)c3ccc(-c4ccccc4-c4ccccc4)cc3)cc21 ; CC1(C)c2ccccc2-c2ccc(N(c3ccc(-c4ccc5c(c4)CCCC5)cc3)c3ccccc3-c3ccccc3)cc21 ; CC1(C)c2ccccc2-c2ccc(N(c3ccc(-c4ccc5c(c4)CCCC5)cc3)c3ccccc3-c3ccccc3)cc21] 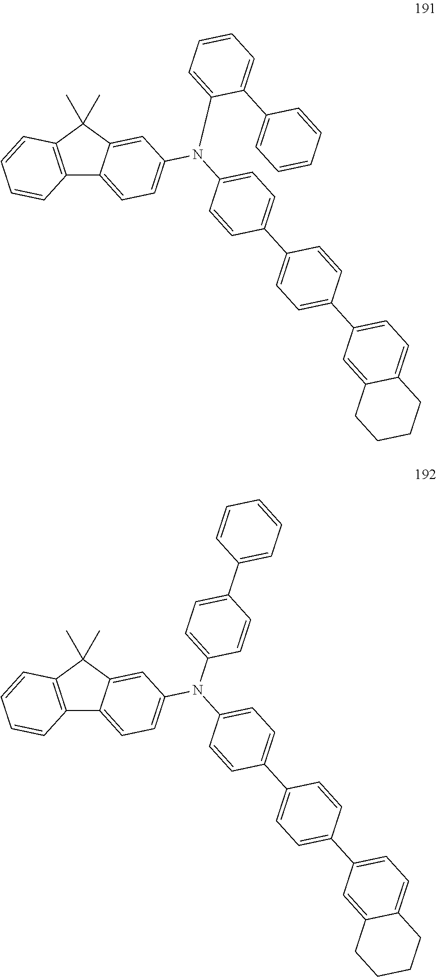 [SMILES: CC1(C)c2ccccc2-c2ccc(N(c3ccc(-c4ccc(-c5ccc6c(c5)CCCC6)cc4)cc3)c3ccccc3-c3ccccc3)cc21 ; CC1(C)c2ccccc2-c2ccc(N(c3ccc(-c4ccccc4)cc3)c3ccc(-c4ccc(-c5ccc6c(c5)CCCC6)cc4)cc3)cc21] 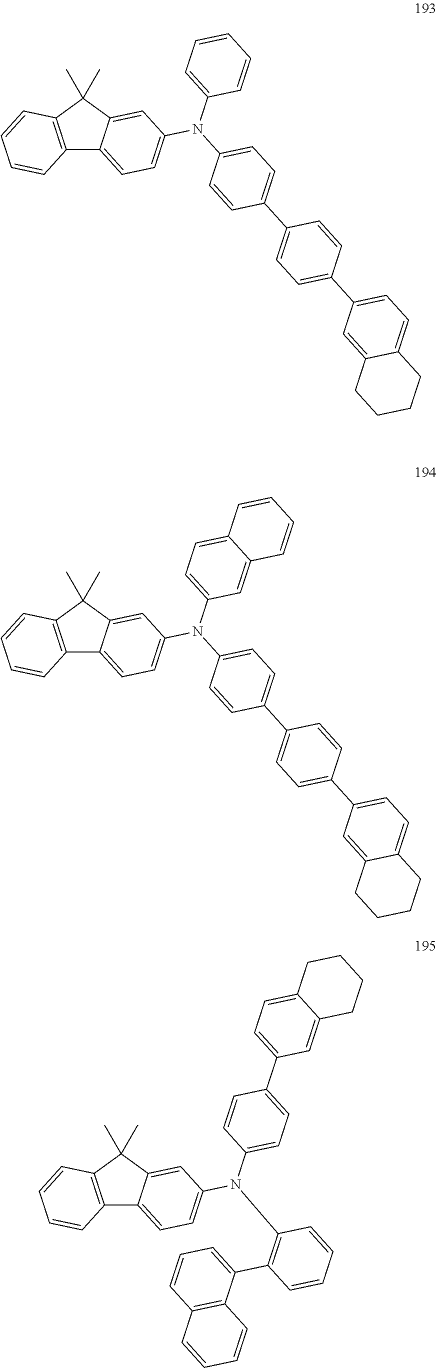 [SMILES: CC1(C)c2ccccc2-c2ccc(N(c3ccccc3)c3ccc(-c4ccc(-c5ccc6c(c5)CCCC6)cc4)cc3)cc21 ; CC1(C)c2ccccc2-c2ccc(N(c3ccc(-c4ccc(-c5ccc6c(c5)CCCC6)cc4)cc3)c3ccc4ccccc4c3)cc21 ; CC1(C)c2ccccc2-c2ccc(N(c3ccc(-c4ccc5c(c4)CCCC5)cc3)c3ccccc3-c3cccc4ccccc34)cc21] 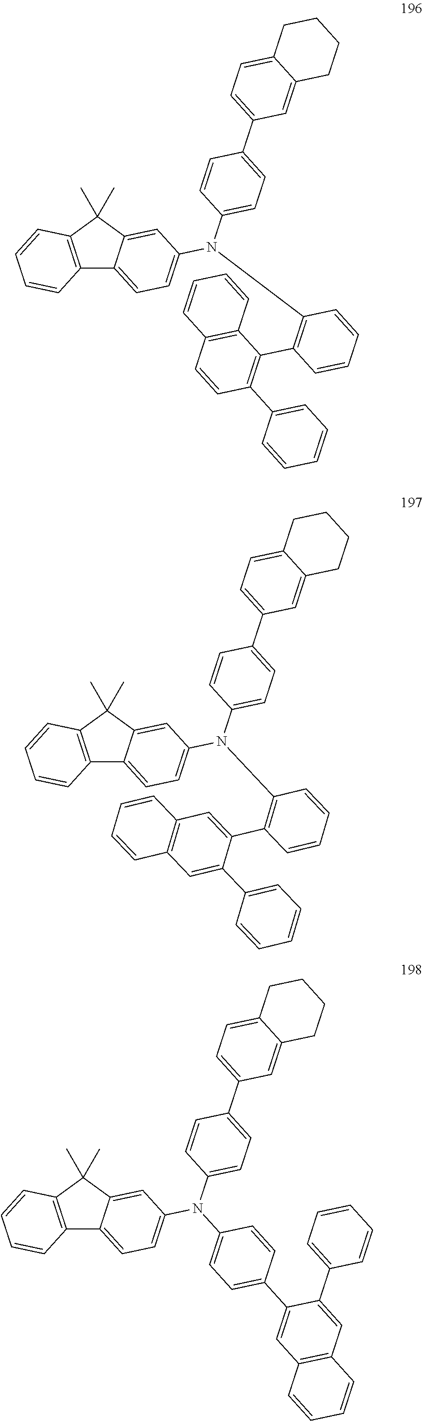 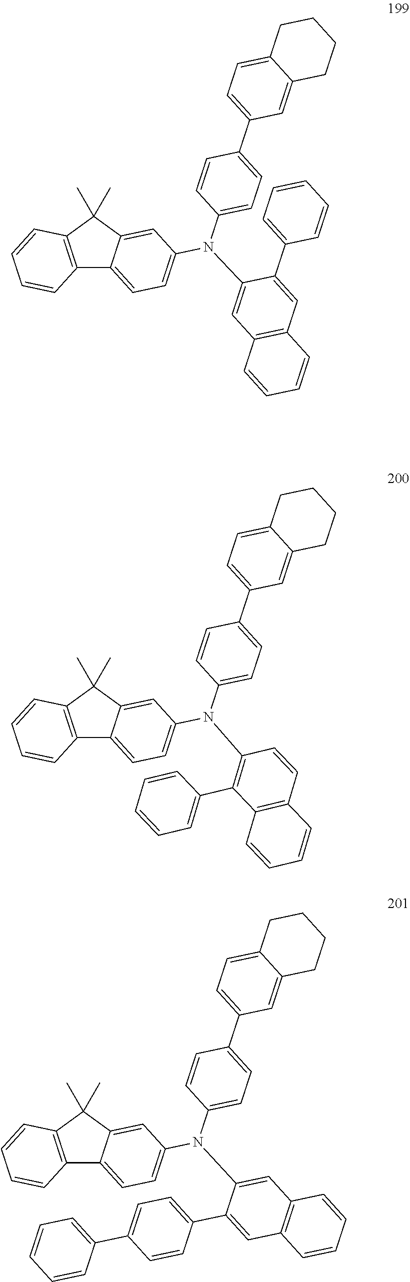 [SMILES: CC1(C)c2ccccc2-c2ccc(N(c3ccc(-c4ccc5c(c4)CCCC5)cc3)c3cc4ccccc4cc3-c3ccccc3)cc21 ; CC1(C)c2ccccc2-c2ccc(N(c3ccc(-c4ccc5c(c4)CCCC5)cc3)c3ccc4ccccc4c3-c3ccccc3)cc21 ; CC1(C)c2ccccc2-c2ccc(N(c3ccc(-c4ccc5c(c4)CCCC5)cc3)c3cc4ccccc4cc3-c3ccc(-c4ccccc4)cc3)cc21] 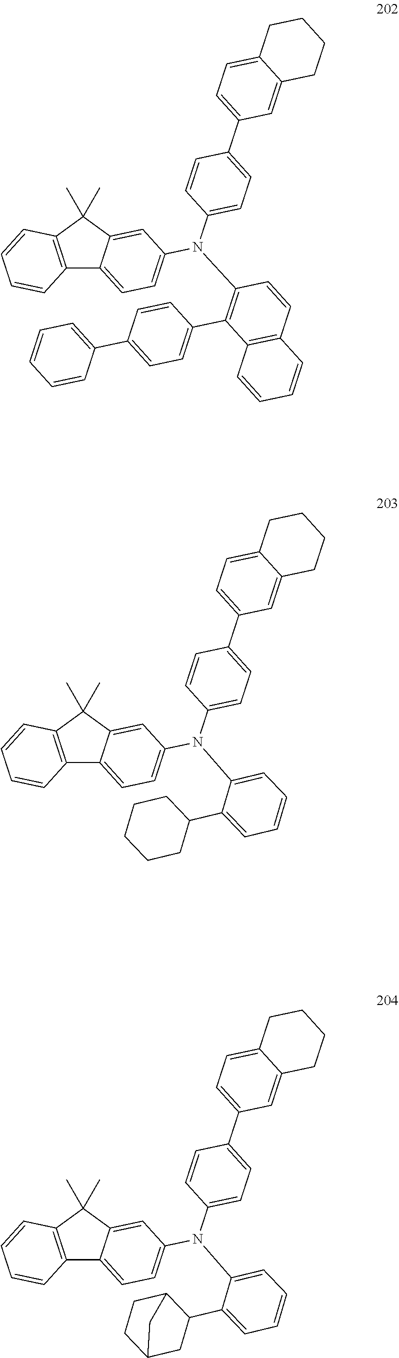 [SMILES: CC1(C)c2ccccc2-c2ccc(N(c3ccc(-c4ccc5c(c4)CCCC5)cc3)c3ccc4ccccc4c3-c3ccc(-c4ccccc4)cc3)cc21 ; CC1(C)c2ccccc2-c2ccc(N(c3ccc(-c4ccc5c(c4)CCCC5)cc3)c3ccccc3C3CCCCC3)cc21 ; CC1(C)c2ccccc2-c2ccc(N(c3ccc(-c4ccc5c(c4)CCCC5)cc3)c3ccccc3C3CC4CCC3C4)cc21] 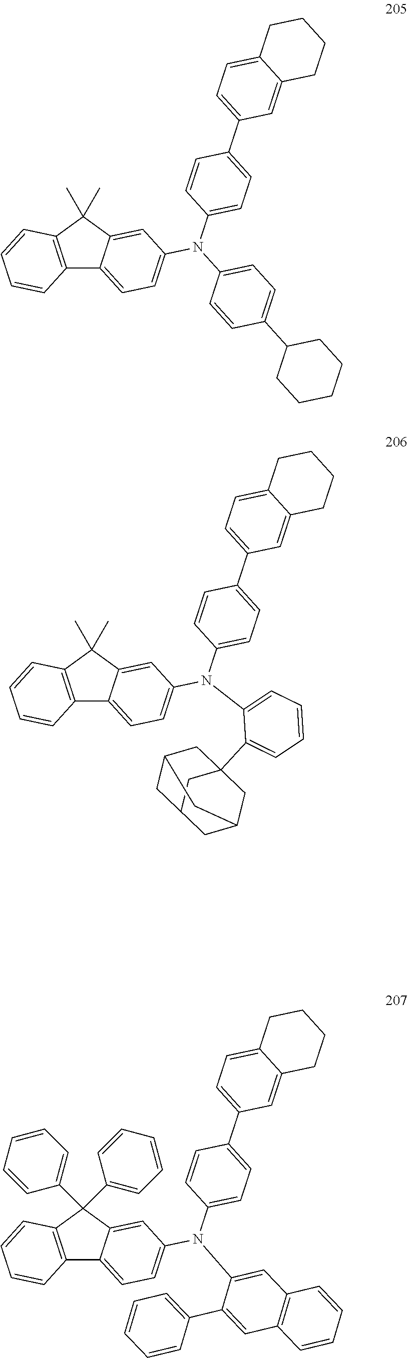 [SMILES: CC1(C)c2ccccc2-c2ccc(N(c3ccc(-c4ccc5c(c4)CCCC5)cc3)c3ccc(C4CCCCC4)cc3)cc21 ; CC1(C)c2ccccc2-c2ccc(N(c3ccc(-c4ccc5c(c4)CCCC5)cc3)c3ccccc3C34CC5CC(CC(C5)C3)C4)cc21 ; c1ccc(-c2cc3ccccc3cc2N(c2ccc(-c3ccc4c(c3)CCCC4)cc2)c2ccc3c(c2)C(c2ccccc2)(c2ccccc2)c2ccccc2-3)cc1] 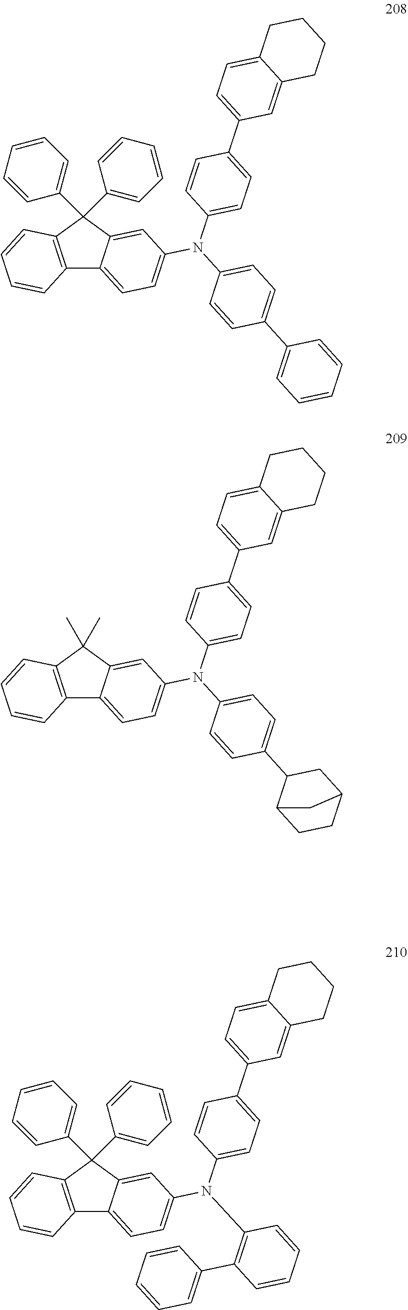 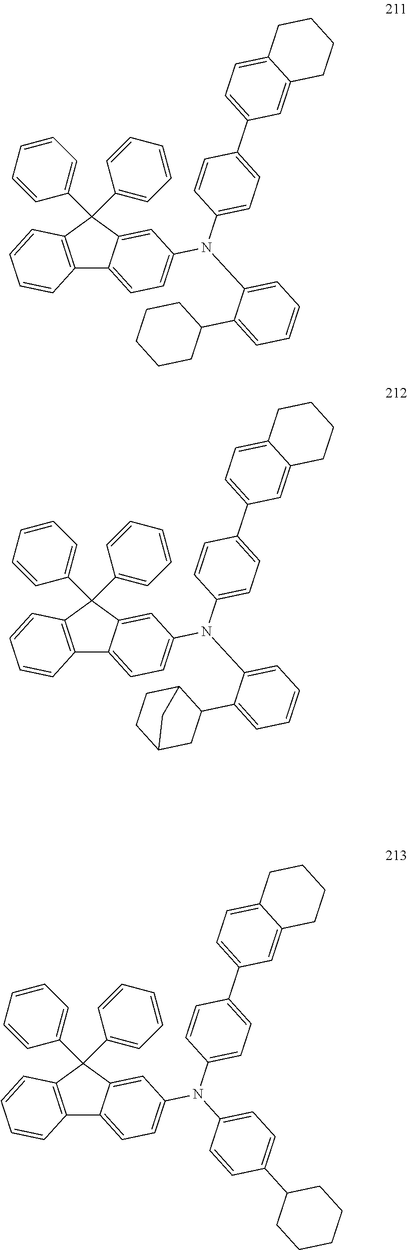 [SMILES: c1ccc(C2(c3ccccc3)c3ccccc3-c3ccc(N(c4ccc(-c5ccc6c(c5)CCCC6)cc4)c4ccccc4C4CCCCC4)cc32)cc1 ; c1ccc(C2(c3ccccc3)c3ccccc3-c3ccc(N(c4ccc(-c5ccc6c(c5)CCCC6)cc4)c4ccccc4C4CC5CCC4C5)cc32)cc1 ; c1ccc(C2(c3ccccc3)c3ccccc3-c3ccc(N(c4ccc(-c5ccc6c(c5)CCCC6)cc4)c4ccc(C5CCCCC5)cc4)cc32)cc1] 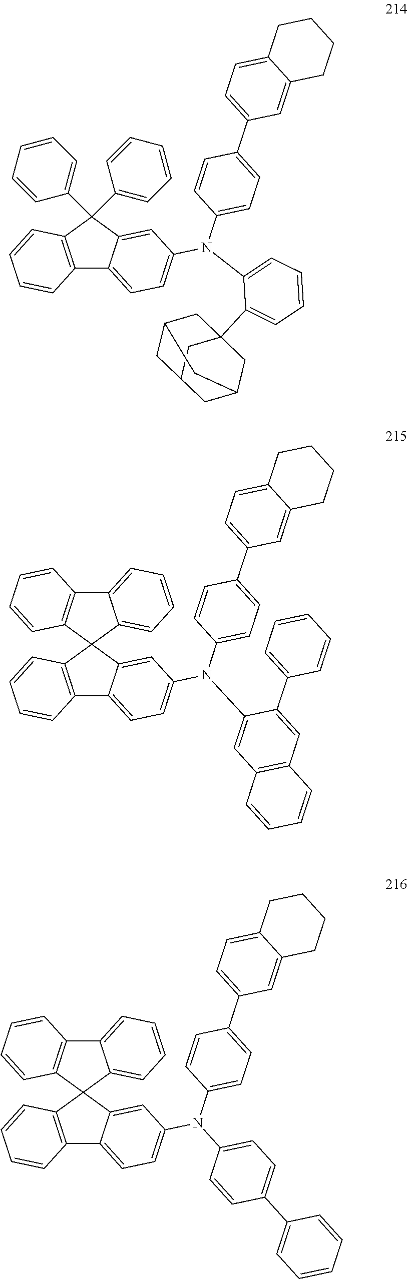 [SMILES: c1ccc(C2(c3ccccc3)c3ccccc3-c3ccc(N(c4ccc(-c5ccc6c(c5)CCCC6)cc4)c4ccccc4C45CC6CC(CC(C6)C4)C5)cc32)cc1 ; c1ccc(-c2cc3ccccc3cc2N(c2ccc(-c3ccc4c(c3)CCCC4)cc2)c2ccc3c(c2)C2(c4ccccc4-c4ccccc42)c2ccccc2-3)cc1 ; c1ccc(-c2ccc(N(c3ccc(-c4ccc5c(c4)CCCC5)cc3)c3ccc4c(c3)C3(c5ccccc5-c5ccccc53)c3ccccc3-4)cc2)cc1] 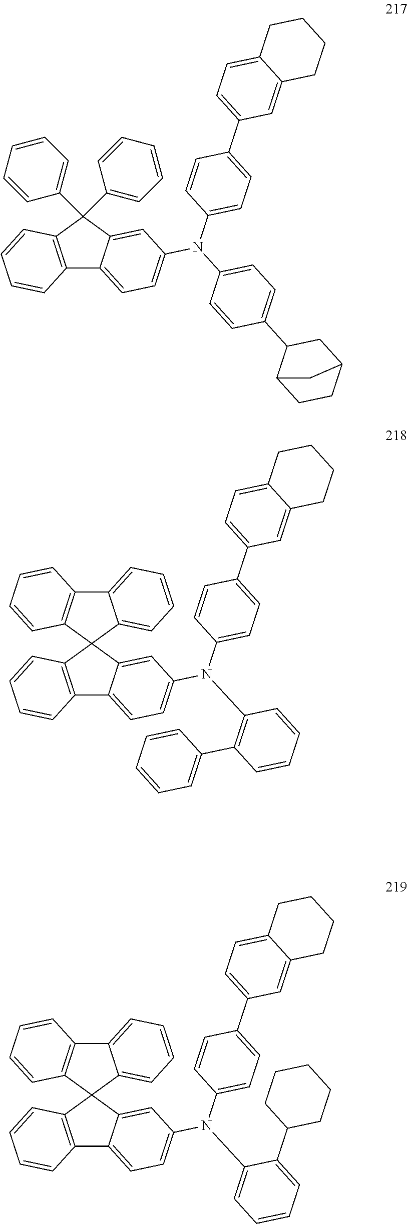 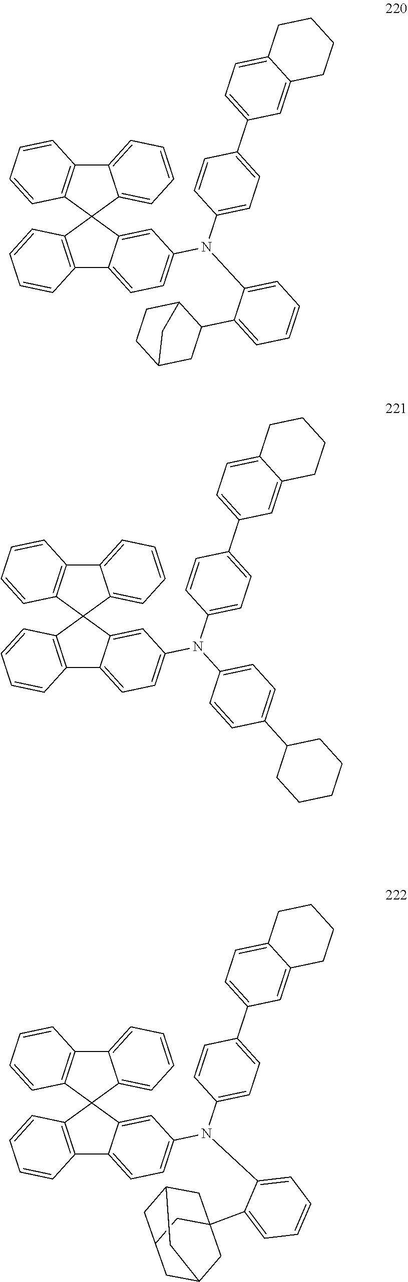 [SMILES: c1ccc2c(c1)-c1ccccc1C21c2ccccc2-c2ccc(N(c3ccc(-c4ccc5c(c4)CCCC5)cc3)c3ccccc3C3CC4CCC3C4)cc21 ; c1ccc2c(c1)-c1ccccc1C21c2ccccc2-c2ccc(N(c3ccc(-c4ccc5c(c4)CCCC5)cc3)c3ccc(C4CCCCC4)cc3)cc21 ; c1ccc2c(c1)-c1ccccc1C21c2ccccc2-c2ccc(N(c3ccc(-c4ccc5c(c4)CCCC5)cc3)c3ccccc3C34CC5CC(CC(C5)C3)C4)cc21] 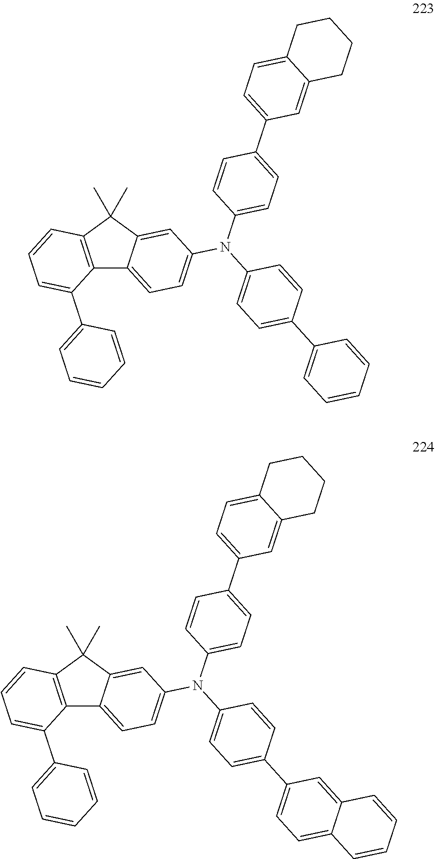 [SMILES: CC1(C)c2cc(N(c3ccc(-c4ccccc4)cc3)c3ccc(-c4ccc5c(c4)CCCC5)cc3)ccc2-c2c(-c3ccccc3)cccc21 ; CC1(C)c2cc(N(c3ccc(-c4ccc5c(c4)CCCC5)cc3)c3ccc(-c4ccc5ccccc5c4)cc3)ccc2-c2c(-c3ccccc3)cccc21] 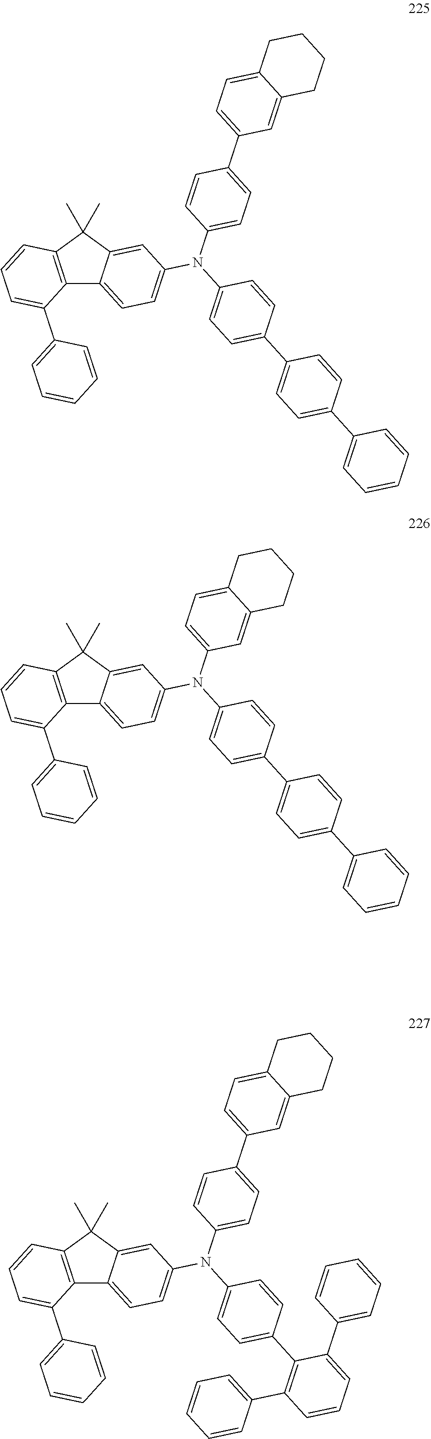 [SMILES: CC1(C)c2cc(N(c3ccc(-c4ccc(-c5ccccc5)cc4)cc3)c3ccc(-c4ccc5c(c4)CCCC5)cc3)ccc2-c2c(-c3ccccc3)cccc21 ; CC1(C)c2cc(N(c3ccc(-c4ccc(-c5ccccc5)cc4)cc3)c3ccc4c(c3)CCCC4)ccc2-c2c(-c3ccccc3)cccc21 ; CC1(C)c2cc(N(c3ccc(-c4ccc5c(c4)CCCC5)cc3)c3ccc(-c4c(-c5ccccc5)cccc4-c4ccccc4)cc3)ccc2-c2c(-c3ccccc3)cccc21] 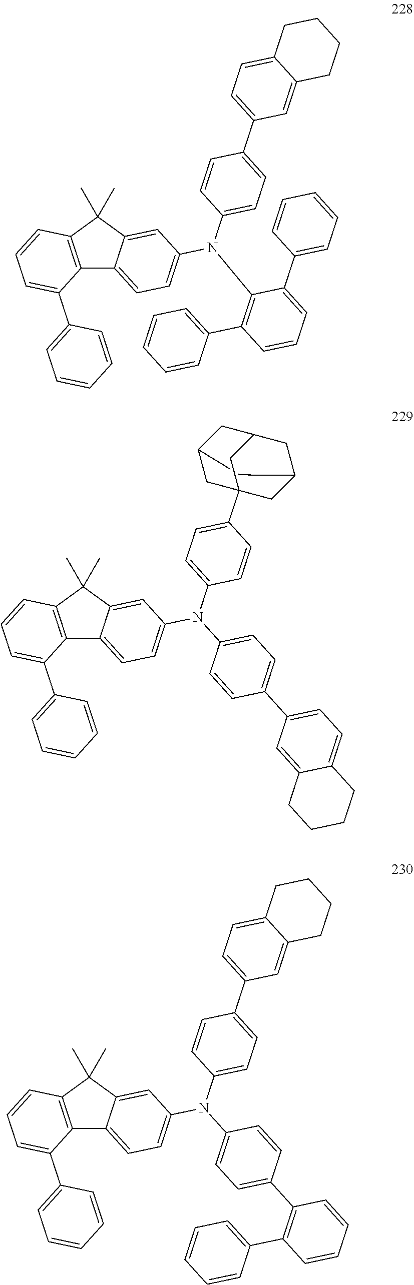 [SMILES: CC1(C)c2cc(N(c3ccc(-c4ccc5c(c4)CCCC5)cc3)c3c(-c4ccccc4)cccc3-c3ccccc3)ccc2-c2c(-c3ccccc3)cccc21 ; CC1(C)c2cc(N(c3ccc(-c4ccc5c(c4)CCCC5)cc3)c3ccc(C45CC6CC(CC(C6)C4)C5)cc3)ccc2-c2c(-c3ccccc3)cccc21 ; CC1(C)c2cc(N(c3ccc(-c4ccc5c(c4)CCCC5)cc3)c3ccc(-c4ccccc4-c4ccccc4)cc3)ccc2-c2c(-c3ccccc3)cccc21] 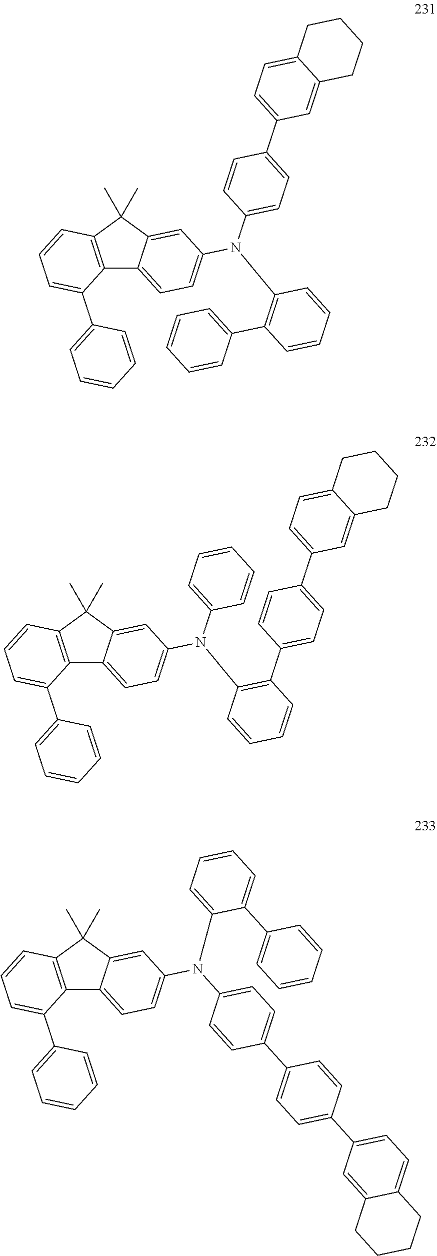 [SMILES: CC1(C)c2cc(N(c3ccc(-c4ccc5c(c4)CCCC5)cc3)c3ccccc3-c3ccccc3)ccc2-c2c(-c3ccccc3)cccc21 ; CC1(C)c2cc(N(c3ccccc3)c3ccccc3-c3ccc(-c4ccc5c(c4)CCCC5)cc3)ccc2-c2c(-c3ccccc3)cccc21 ; CC1(C)c2cc(N(c3ccc(-c4ccc(-c5ccc6c(c5)CCCC6)cc4)cc3)c3ccccc3-c3ccccc3)ccc2-c2c(-c3ccccc3)cccc21] 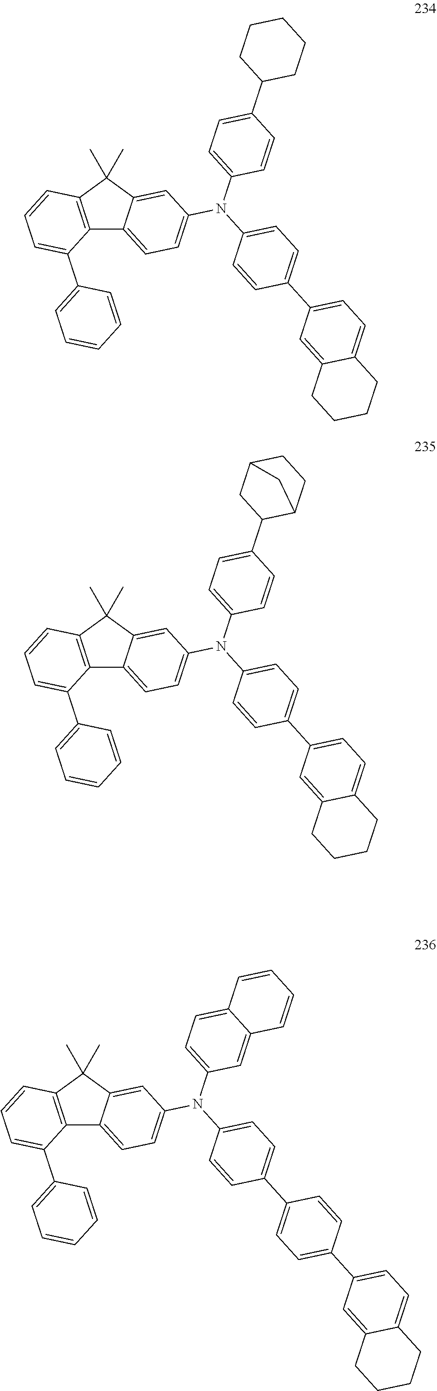 [SMILES: CC1(C)c2cc(N(c3ccc(-c4ccc5c(c4)CCCC5)cc3)c3ccc(C4CCCCC4)cc3)ccc2-c2c(-c3ccccc3)cccc21 ; CC1(C)c2cc(N(c3ccc(-c4ccc5c(c4)CCCC5)cc3)c3ccc(C4CC5CCC4C5)cc3)ccc2-c2c(-c3ccccc3)cccc21 ; CC1(C)c2cc(N(c3ccc(-c4ccc(-c5ccc6c(c5)CCCC6)cc4)cc3)c3ccc4ccccc4c3)ccc2-c2c(-c3ccccc3)cccc21] 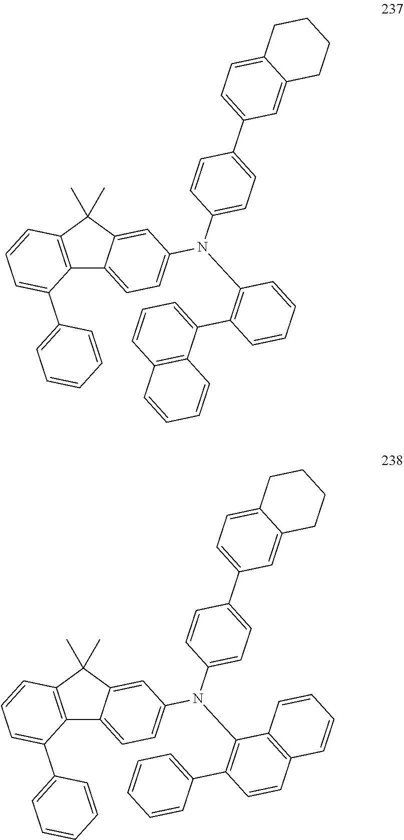 [SMILES: CC1(C)c2cc(N(c3ccc(-c4ccc5c(c4)CCCC5)cc3)c3ccccc3-c3cccc4ccccc34)ccc2-c2c(-c3ccccc3)cccc21 ; CC1(C)c2cc(N(c3ccc(-c4ccc5c(c4)CCCC5)cc3)c3c(-c4ccccc4)ccc4ccccc34)ccc2-c2c(-c3ccccc3)cccc21] 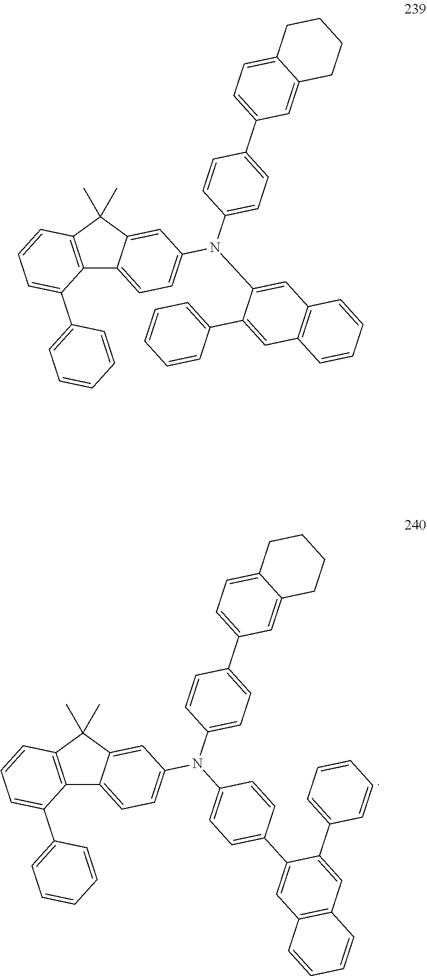 [SMILES: CC1(C)c2cc(N(c3ccc(-c4ccc5c(c4)CCCC5)cc3)c3cc4ccccc4cc3-c3ccccc3)ccc2-c2c(-c3ccccc3)cccc21 ; CC1(C)c2cc(N(c3ccc(-c4ccc5c(c4)CCCC5)cc3)c3ccc(-c4cc5ccccc5cc4-c4ccccc4)cc3)ccc2-c2c(-c3ccccc3)cccc21]
